(19) United States
(12) Patent Application Publication
(10) Pub. No.: US 2016/0204357 A1
(43) Pub. Date: Jul. 14, 2016

(54) POLYCYCLIC COMPOUND AND ORGANIC LIGHT EMITTING DEVICE USING THE SAME

(71) Applicant: HEESUNG MATERIAL LTD., Gyeonggi-do (KR)

(72) Inventors: So-Hyun JANG, Seoul (KR); Kee-Yong KIM, Suwon-city (KR); Jin-Seok CHOI, Suwon-city (KR); Dae-Hyuk CHOI, Yongin-city (KR); Sung-Jin EUM, Yongin-city (KR); Joo-Dong LEE, Seongnam-city (KR)

(73) Assignee: HEESUNG MATERIAL LTD., Yongin-City, Gyeonggi-do (KR)

(21) Appl. No.: 14/898,348

(22) PCT Filed: Jun. 29, 2015
(86) PCT No.: PCT/KR2015/006626

§ 371 Date: Dec. 14, 2015

(30) Foreign Application Priority Data
Jun. 27, 2014 (KR) 10-2014-0080248
Sep. 23, 2014 (KR) 10-2014-0127226
Mar. 25, 2015 (KR) 10-2015-0041326

(51) Int. Cl. H01L 51/00; C07F 9/576; C07D 401/10; C09K 11/02; C07D 401/14; C07D 471/04; C07D 417/10; C07D 221/18; C07D 487/04

(52) U.S. Cl. CPC H01L 51/0072; C07D 221/18; H01L 51/0052; C07F 9/5765; C07D 401/10; H01L 51/0067; C07D 487/04; C07D 401/14; C07D 471/04; C07D 417/10; H01L 51/0071; H01L 51/0058; C09K 11/025; H01L 51/5072

(57) ABSTRACT

Disclosed are a novel polycyclic compound and an organic light emitting device using the same.

### CROSS-REFERENCE TO RELATED APPLICATIONS

This application claims priority to and the benefit of Korean Patent Application No. 10-2014-0080248, 10-2014-0127226, and 10-2015-0041326 filed in the Korean Intellectual Property Office on Jun. 27, 2014, Sep. 23, 2014, and Mar. 25, 2015, respectively, the entire contents of which are incorporated herein by reference.

### TECHNICAL FIELD

The present invention relates to a novel polycyclic compound and an organic light emitting device using the same.

### BACKGROUND ART

An electroluminescent device is a self-luminous display device, and has advantages in that the device has a wide viewing angle, an excellent contrast, and quick response time.

An organic light emitting device has a structure in which an organic thin film is arranged between two electrodes. When voltage is applied to an organic light emitting device having such a structure, light is emitted by electrons and holes injected from the two electrodes being dissipated after the electrons and holes are bonded and make a pair in the organic thin film. The organic thin film may be formed as monolayer or a multilayer as necessary.

Materials of the organic thin film may have a light emitting function as necessary. For example, as a material of the organic thin film, compounds capable of forming a light emitting layer alone may be used, or compounds capable of performing as a host or a dopant of a host-dopant-based light emitting layer may also be used. In addition to these, compounds capable of performing hole injection, hole transport, electron blocking, hole blocking, electron transport, electron injection, or the like, may also be used as a material of the organic thin film.

There have been continuous demands for the development of organic thin film materials in order to improve the performance, life span, or efficiency of an organic light emitting device.

### SUMMARY OF THE INVENTION

The present invention has been made in an effort to provide a novel polycyclic compound and an organic light emitting device using the same.

An exemplary embodiment of the present invention provides a compound of the following chemical formula 1:

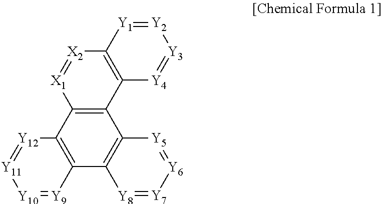

wherein in the chemical formula 1,

X₁ and X₂ are the same as or different from each other, and are each independently N or CR₁,

Y₁ to Y₁₂ are the same as or different from each other, and are each independently N or CR,

R and R₁ are the same as or different from each other, and are each independently one selected from the group consisting of hydrogen; deuterium; halogen; linear or branched substituted or unsubstituted C₁ to C₆₀ alkyl; linear or branched substituted or unsubstituted C₂ to C₆₀ alkenyl; linear or branched substituted or unsubstituted C₂ to C₆₀ alkynyl; linear or branched substituted or unsubstituted C₁ to C₆₀ alkoxy; substituted or unsubstituted C₃ to C₆₀ monocyclic or polycyclic cycloalkyl; substituted or unsubstituted C₂ to C₆₀ monocyclic or polycyclic heterocycloalkyl; substituted or unsubstituted C₆ to C₆₀ monocyclic or polycyclic aryl; substituted or unsubstituted C₂ to C₆₀ monocyclic or polycyclic heteroaryl; —SiR₁₁R₁₂R₁₃; —P(═O)R₁₄R₁₅; and amine unsubstituted or substituted with C₁ to C₂₀ alkyl, substituted or unsubstituted C₆ to C₆₀ monocyclic or polycyclic aryl, or substituted or unsubstituted C₂ to C₆₀ monocyclic or polycyclic heteroaryl, or at least two adjacent Rs are bonded to each other to form a substituted or unsubstituted monocyclic or polycyclic aliphatic or aromatic hydrocarbon ring;

R₁₁ to R₁₅ are the same as or different from each other, and are each independently one selected from the group consisting of linear or branched substituted or unsubstituted C₁ to C₆₀ alkyl; substituted or unsubstituted C₃ to C₆₀ monocyclic or polycyclic cycloalkyl; substituted or unsubstituted C₆ to C₆₀ monocyclic or polycyclic aryl; and substituted or unsubstituted C₂ to C₆₀ monocyclic or polycyclic heteroaryl.

Another exemplary embodiment of the present invention provides an organic light emitting device including: an anode; a cathode; and one or more organic material layers provided between the anode and the cathode, wherein one or more of the organic material layers include the compound of the chemical formula 1.

According to the exemplary embodiments of the present invention, a compound described in the present specification may be used as a material of an organic material layer of an organic light emitting device. The compound may be used as a hole injection material, a hole transport material, a light emitting material, a hole blocking material, an electron transport material, an electron injection material, or the like, in an organic light emitting device. In particular, the compound of the chemical formula 1 may be used as a material of one or more of a light emitting layer, an electron transport layer, and a hole blocking layer in an organic light emitting device.

### BRIEF DESCRIPTION OF THE DRAWINGS

FIGS. 1 to 3 illustrate examples of laminating order of electrodes and organic material layers of an organic light emitting device according to exemplary embodiments of the present invention.

FIG. 4 is a graph illustrating a PL spectrum of a compound 16 at a wavelength of 302 nm.

FIG. 5 is a graph illustrating an LTPL spectrum of the compound 16 at a wavelength of 382 nm.

FIG. 6 is a graph illustrating a PL spectrum of a compound 18 at a wavelength of 286 nm.

FIG. 7 is a graph illustrating an LTPL spectrum of the compound 18 at a wavelength of 383 nm.

FIG. 8 is a graph illustrating a PL spectrum of a compound 22 at a wavelength of 258 nm.

FIG. 9 is a graph illustrating an LTPL spectrum of the compound 22 at a wavelength of 283 nm.

FIG. 10 is a graph illustrating a PL spectrum of a compound 37 at a wavelength of 261 nm.

FIG. 11 is a graph illustrating an LTPL spectrum of the compound 37 at a wavelength of 308 nm.

FIG. 12 is a graph illustrating a PL spectrum of a compound 41 at a wavelength of 261 nm.

FIG. 13 is a graph illustrating an LTPL spectrum of the compound 41 at a wavelength of 308 nm.

FIG. 14 is a graph illustrating a PL spectrum of a compound 43 at a wavelength of 262 nm.

FIG. 15 is a graph illustrating an LTPL spectrum of the compound 43 at a wavelength of 311 nm.

FIG. 16 is a graph illustrating a PL spectrum of a compound 44 at a wavelength of 270 nm.

FIG. 17 is a graph illustrating an LTPL spectrum of the compound 44 at a wavelength of 311 nm.

FIG. 18 is a graph illustrating a PL spectrum of a compound 73 at a wavelength of 284 nm.

FIG. 19 is a graph illustrating an LTPL spectrum of the compound 73 at a wavelength of 383 nm.

FIG. 20 is a graph illustrating a PL spectrum of a compound 92 at a wavelength of 268 nm.

FIG. 21 is a graph illustrating an LTPL spectrum of the compound 92 at a wavelength of 383 nm.

FIG. 22 is a graph illustrating a PL spectrum of a compound 294 at a wavelength of 265 nm.

FIG. 23 is a graph illustrating an LTPL spectrum of the compound 294 at a wavelength of 299 nm.

FIG. 24 is a graph illustrating a PL spectrum of a compound 324 at a wavelength of 260 nm.

FIG. 25 is a graph illustrating an LTPL spectrum of the compound 324 at a wavelength of 320 nm.

FIG. 26 is a graph illustrating a PL spectrum of a compound 560 at a wavelength of 273 nm.

FIG. 27 is a graph illustrating an LTPL spectrum of the compound 560 at a wavelength of 353 nm.

FIG. 28 is a graph illustrating a PL spectrum of a compound 563 at a wavelength of 273 nm.

FIG. 29 is a graph illustrating an LTPL spectrum of the compound 563 at a wavelength of 353 nm.

FIG. 30 is a graph illustrating a PL spectrum of a compound 568 at a wavelength of 253 nm.

FIG. 31 is a graph illustrating an LTPL spectrum of the compound 568 at a wavelength of 348 nm.

FIG. 32 is a graph illustrating a PL spectrum of a compound 603.

FIG. 33 is a graph illustrating an LTPL spectrum of the compound 603.

FIG. 34 is a graph illustrating a PL spectrum of a compound 604.

FIG. 35 is a graph illustrating an LTPL spectrum of the compound 604.

### EXPLANATION OF REFERENCE NUMERALS AND SYMBOLS

100: Substrate
200: Positive electrode
300: Organic material layer
301: Hole injection layer
302: Hole transport layer
303: Light emitting layer
304: Hole blocking layer
305: Electron transport layer
306: Electron injection layer
400: Negative electrode

### DETAILED DESCRIPTION

Hereinafter, the present invention will be described in detail. A compound described in the present specification may be expressed by the chemical formula 1. To be specific, the compound of the chemical formula 1 may be used as a material of an organic material layer of an organic light emitting device due to the above-described structural properties of a core structure and a substituent.

In the present specification, the term “substituted or unsubstituted” refers to a group that may be substituted or may not be further substituted with one or more substituents selected from the group consisting of deuterium; halogen; linear or branched C₁ to C₆₀ alkyl; linear or branched C₂ to C₆₀ alkenyl; linear or branched C₂ to C₆₀ alkynyl; C₃ to C₆₀ monocyclic or polycyclic cycloalkyl; C₂ to C₆₀ monocyclic or polycyclic heterocycloalkyl; C₆ to C₆₀ monocyclic or polycyclic aryl; C₂ to C₆₀ monocyclic or polycyclic heteroaryl; —SiR₁₁R₁₂R₁₃; —P(═O)R₁₄R₁₅; C₁ to C₂₀ alkylamine; C₆ to C₆₀ monocyclic or polycyclic arylamine; and C₂ to C₆₀ monocyclic or polycyclic heteroarylamine, or a substituent bonded to two or more selected from the substituents. For example, “the substituent bonded to two or more substituents” may be a biphenyl group. That is, the biphenyl group may be an aryl group or can be construed as a substituent bonded to two phenyl groups. The R₁₁ to R₁₅ are the same as or different from each other, and are each independently one selected from the group consisting of hydrogen; deuterium; linear or branched C₁ to C₆₀ alkyl substituted or unsubstituted with linear or branched C₁ to C₆₀ alkyl, C₆ to C₆₀ monocyclic or polycyclic aryl, or C₂ to C₆₀ monocyclic or polycyclic heteroaryl; C₆ to C₆₀ monocyclic or polycyclic aryl unsubstituted or substituted with linear or branched C₁ to C₆₀ alkyl, C₆ to C₆₀ monocyclic or polycyclic aryl, or C₂ to C₆₀ monocyclic or polycyclic heteroaryl; and C₂ to C₆₀ monocyclic or polycyclic heteroaryl unsubstituted or substituted with linear or branched C₁ to C₆₀ alkyl, C₆ to C₆₀ monocyclic or polycyclic aryl, or C₂ to C₆₀ monocyclic or polycyclic heteroaryl.

In the present specification, the “adjacent” group may refer to a substituent substituted at an atom directly bonded to an atom substituted with substituent, a substituent sterically closest to the substituent, or another substituent substituted at an atom substituted with the substituent. For example, two substituents substituted at ortho positions in a benzene ring and two substituents substituted at the same carbon atom in an aliphatic ring may be construed as the “adjacent” groups.

According to an exemplary embodiment of the present invention, the term “substituted or unsubstituted” refers to a group that may be substituted or may not be further substituted with one or more substituents selected from the group consisting of deuterium; halogen; linear or branched C₁ to C₆₀ alkyl; C₆ to C₆₀ monocyclic or polycyclic aryl; C₂ to C₆₀ monocyclic or polycyclic heteroaryl; —SiR₁₁R₁₂R₁₃; and —P(═O)R₁₄R₁₅, or a substituent bonded to two or more selected from the substituents, and

The R₁₁ to R₁₅ are the same as or different from each other, and are each independently one selected from the group consisting of hydrogen; deuterium; linear or branched C₁ to C₆₀ alkyl unsubstituted or substituted with linear or branched C₁ to C₆₀ alkyl, C₆ to C₆₀ monocyclic or polycyclic aryl, or C₂ to C₆₀ monocyclic or polycyclic heteroaryl; C₆ to C₆₀ monocyclic or polycyclic aryl unsubstituted or substituted with linear or branched C₁ to C₆₀ alkyl, C₆ to C₆₀ monocyclic or polycyclic aryl, or C₂ to C₆₀ monocyclic or polycyclic heteroaryl; and C₂ to C₆₀ monocyclic or polycyclic heteroaryl unsubstituted or substituted with linear or branched C₁ to C₆₀ alkyl, C₆ to C₆₀ monocyclic or polycyclic aryl, or C₂ to C₆₀ monocyclic or polycyclic heteroaryl.

In the present specification, halogen may be fluorine, chlorine, bromine, or iodine.

In the present specification, alkyl includes linear or branched alkyl having 1 to 60 carbon atoms, and may be further substituted with other substituents. The number of carbon atoms of the alkyl may be 1 to 60, specifically 1 to 40, and more specifically 1 to 20.

In the present specification, alkenyl includes linear or branched alkenyl having 2 to 60 carbon atoms, and may be further substituted with other substituents. The number of carbon atoms of the alkenyl may be 2 to 60, specifically 2 to 40, and more specifically 2 to 20.

In the present specification, alkynyl includes linear or branched alkynyl having 2 to 60 carbon atoms, and may be further substituted with other substituents. The number of carbon atoms of the alkynyl may be 2 to 60, specifically 2 to 40, and more specifically 2 to 20.

In the present specification, cycloalkyl includes monocyclic or polycyclic cycloalkyl having 3 to 60 carbon atoms, and may be further substituted with other substituents. Herein, the term “polycyclic” means a group in which cycloalkyl is directly bonded to or fused with other ring groups. Herein, the term “other ring groups” may be cycloalkyl, but may also be other types of ring groups, for example, heterocycloalkyl, aryl, heteroaryl, or the like. The number of carbon atoms of the cycloalkyl may be 3 to 60, specifically 3 to 40, and more specifically 5 to 20.

In the present specification, heterocycloalkyl includes one or more of 0, S, Se, N, and Si as a heteroatom, includes monocyclic or polycyclic heterocycloalkyl having 2 to 60 carbon atoms, and may be further substituted with other substituents. Herein, the term “polycyclic” means a group in which heterocycloalkyl is directly bonded to or fused with other ring groups. Herein, the term “other ring groups” may be heterocycloalkyl, but may also be other types of ring groups, for example, cycloalkyl, aryl, heteroaryl, or the like. The number of carbon atoms of the heterocycloalkyl may be 2 to 60, specifically 2 to 40, and more specifically 3 to 20.

In the present specification, aryl includes monocyclic or polycyclic aryl having 6 to 60 carbon atoms, and may be further substituted with other substituents. Herein, the term “polycyclic” means a group in which aryl is directly bonded to or fused with other ring groups. Herein, the term “other ring groups” may be aryl, but may also be other types of ring groups, for example, cycloalkyl, heterocycloalkyl, heteroaryl, or the like. The aryl includes a spiro group. The number of carbon atoms of the aryl may be 6 to 60, specifically 6 to 40, and more specifically 6 to 25. Specific examples of the aryl include phenyl, biphenyl, triphenyl, naphthyl, anthryl, chrysenyl, phenanthrenyl, perylenyl, fluoranthenyl, triphenylenyl, phenalenyl, pyrenyl, tetracenyl, pentacenyl, indenyl, acenaphthylenyl, fluorenyl, benzofluorenyl; benzochrysenyl, spirobifluorenyl, dibenzophenanthridinyl, or fused rings thereof, but are not limited thereto.

In the present specification, the spiro group is a group including a spiro structure, and may have 15 to 60 carbon atoms. For example, the Spiro group may include a structure in which a 2,3-dihydro-1H-indene group or a cyclohexane group is spiro-bonded to a fluorene group. To be specific, the spiro group includes a group of the following structural formulas.

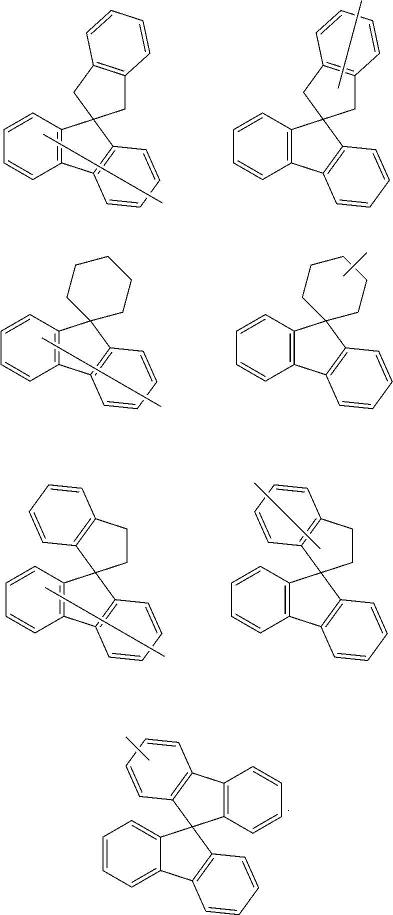

In the present specification, heteroaryl includes one or more of S, O, Se, N, and Si as a heteroatom, includes monocyclic or polycyclic heteroaryl having 2 to 60 carbon atoms, and may be further substituted with other substituents. Herein, the term “polycyclic” means a group in which heteroaryl is directly bonded to or fused with other ring groups. Herein, the term “other ring groups” may be heteroaryl, but may also be other types of ring groups, for example, cycloalkyl, heterocycloalkyl, aryl, or the like. The number of carbon atoms of the heteroaryl may be 2 to 60, specifically 2 to 40, and more specifically 3 to 25. Specific examples of the heteroaryl include pyridyl, pyrolyl, pyrimidyl, pyridazinyl, furanyl, a thiophene group, imidazolyl, pyrazolyl, oxazolyl, isoxazolyl, thiazolyl, isothiazolyl, triazolyl, furazanyl, oxadiazolyl, thiadiazolyl, dithiazolyl, tetrazolyl, pyranyl, thiopyranyl, diazinyl, oxazinyl, thiazinyl, dioxynyl, triazinyl, tetrazinyl, quinolyl, isoquinolyl, quinazolinyl, isoquinazolinyl, naphtyridyl, acridinyl, phenanthridinyl, imidazopyridinyl, diazanaphthalenyl, triazaindene, indolyl, indolizinyl, benzothiazolyl, benzoxazolyl, benzimidazolyl, a benzothiophene group, a benzofuran group, a dibenzothiophene group, a dibenzofuran group, carbazolyl, benzocarbazolyl, dibenzocarbazolyl, phenazinyl, dibenzoxylol, spirobi(dibenzoxylol), dihydrophenazinyl, phenoxazinyl, phenanthridyl, or fused rings thereof, but are not limited thereto.

In the present specification, if at least two adjacent Rs are bonded to each other to form a ring, the ring may be a monocyclic or polycyclic substituted or unsubstituted aliphatic or aromatic hydrocarbon ring.

In the present specification, an aliphatic hydrocarbon ring includes a monocyclic or polycyclic saturated hydrocarbon ring having 3 to 30 carbon atoms, and the above explanation of cycloalkyl can be applied thereto except that adjacent substituents are bonded to each other to form a ring.

According to an exemplary embodiment of the present invention, the chemical formula 1 is represented by the following chemical formula 2 or 3:

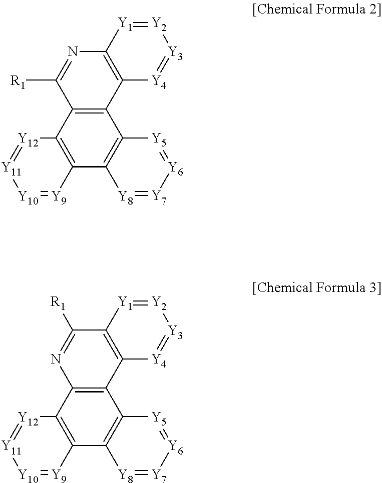

wherein in the chemical formulas 2 and 3,

Y₁ to Y₁₂ are the same as or different from each other, and are each independently N or CR, and

R, and R₁ are the same as defined in the chemical formula 1.

According to an exemplary embodiment of the present invention, in the chemical formula 1, if two or more of the Y₁ to Y₁₄ are CR, the multiple Rs are the same as or different from each other.

According to an exemplary embodiment of the present invention, in the chemical formula 1, all of the Y₁ to Y₁₄ are CR, or any one of the Y₁ to Y₁₂ is N and the others are CR, and the R is the same as defined in the chemical formula 1.

According to an exemplary embodiment of the present invention, in the chemical formula 1, all of the Y₁ to Y₄ are CR, or any one of the Y₁ to Y₄ is N and the others are CR, and the R is the same as defined in the chemical formula 1.

According to an exemplary embodiment of the present invention, in the chemical formula 1, all of the Y₅ to Y₈ are CR, or any one of the Y₅ to Y₈ is N and the others are CR, and the R is the same as defined in the chemical formula 1.

According to an exemplary embodiment of the present invention, in the chemical formula 1, all of the Y₉ to Y₁₂ are CR, or any one of the Y₉ to Y₁₂ is N and the others are CR, and the R is the same as defined in the chemical formula 1.

According to an exemplary embodiment of the present invention, in the chemical formula 1, the Y₁ to Y₁₂ are CR, and the R is the same as defined in the chemical formula 1.

According to an exemplary embodiment of the present invention, in the chemical formula 1, all of the Y₁ to Y₁₄ are CR, or any one of the Y₁ to Y₁₂ is N and the others are CR, and the R is hydrogen or deuterium.

According to an exemplary embodiment of the present invention, in the chemical formula 1, all of the Y₁ to Y₄ are CR, or any one of the Y₁ to Y₄ is N and the others are CR, and the R is hydrogen or deuterium.

According to an exemplary embodiment of the present invention, in the chemical formula 1, all of the Y₅ to Y₈ are CR, or any one of the Y₅ to Y₈ is N and the others are CR, and the R is hydrogen or deuterium.

According to an exemplary embodiment of the present invention, in the chemical formula 1, all of the Y₉ to Y₁₂ are CR, or any one of the Y₉ to Y₁₂ is N and the others are CR, and the R is hydrogen or deuterium.

According to an exemplary embodiment of the present invention, in the chemical formula 1, the Y₁ to Y₁₂ are CR, and the R is hydrogen or deuterium.

According to an exemplary embodiment of the present invention, the chemical formula 1 can be represented by any one of the following chemical formulas 6 to 8.

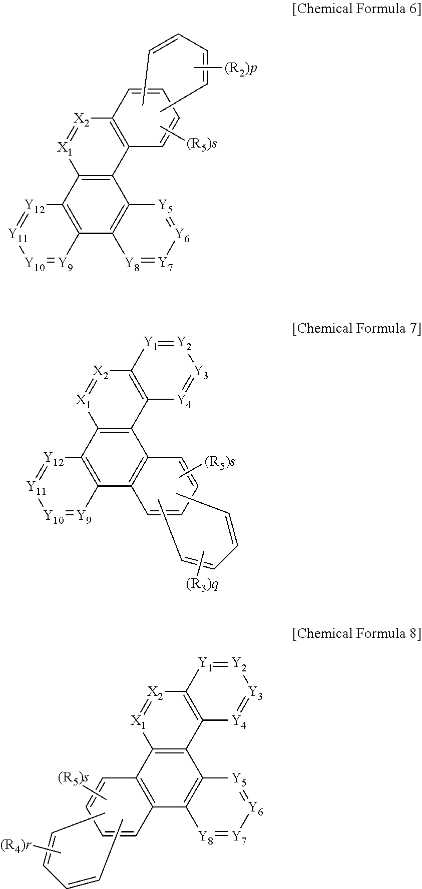

In the chemical formulas 6 to 8,

X₁ and X₂ are the same as defined in the chemical formula 1,

R₂ to R₅ are the same as or different from each other, and are each independently one selected from the group consisting of hydrogen; halogen; linear or branched substituted or unsubstituted C₁ to C₆₀ alkyl; linear or branched substituted or unsubstituted C₂ to C₆₀ alkenyl; linear or branched substituted or unsubstituted C₂ to C₆₀ alkynyl; linear or branched substituted or unsubstituted C₁ to C₆₀ alkoxy; substituted or unsubstituted C₃ to C₆₀ monocyclic or polycyclic cycloalkyl; substituted or unsubstituted C₂ to C₆₀ monocyclic or polycyclic heterocycloalkyl; substituted or unsubstituted C₆ to C₆₀ monocyclic or polycyclic aryl; substituted or unsubstituted C₂ to C₆₀ monocyclic or polycyclic heteroaryl; and amine unsubstituted or substituted with C₁ to C₂₀ alkyl, substituted or unsubstituted C₆ to C₆₀ monocyclic or polycyclic aryl, or substituted or unsubstituted C₂ to C₆₀ monocyclic or polycyclic heteroaryl,

r, p, and q are independently integers of 0 to 4, and

s is an integer of 0 to 2.

According to an exemplary embodiment of the present invention, in the chemical formulas 6 to 8, R₂ to R₅ are independently hydrogen or deuterium.

According to an exemplary embodiment of the present invention, in the chemical formulas 6 to 8, p, q, r, and s are 0.

According to an exemplary embodiment of the present invention, the chemical formula 1 can be represented by any one of the following chemical formulas 6-1 to 8-1.

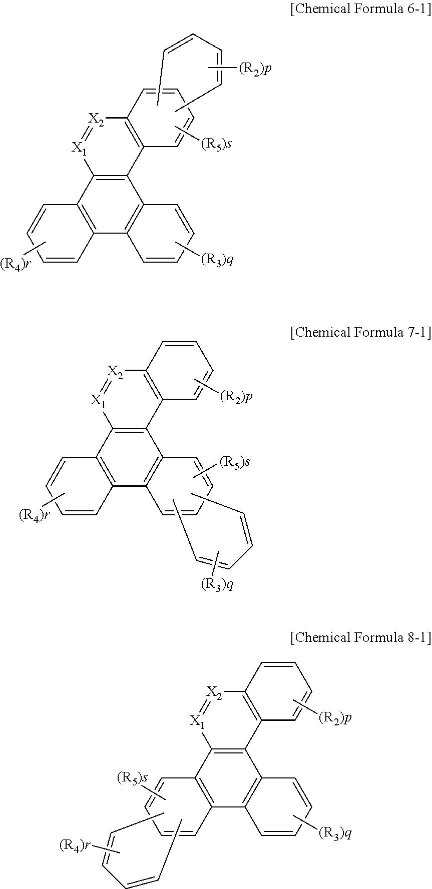

In the chemical formulas 6-1 to 8-1,

X₁, X₂, R₂ to R₅, r, s, p, and q are the same as defined in the chemical formulas 6 to 8.

According to an exemplary embodiment of the present invention, in the chemical formula 1, R₁ is substituted or unsubstituted C₆ to C₆₀ monocyclic or polycyclic aryl or substituted or unsubstituted C₂ to Cₒ₆ monocyclic or polycyclic heteroaryl.

According to an exemplary embodiment of the present invention, in the chemical formulas 2 to 3,

R₁ is -(L)m-(Z)n,

L is substituted or unsubstituted C₆ to C₆₀ monocyclic or polycyclic arylene; or substituted or unsubstituted C₂ to C₆₀ monocyclic or polycyclic heteroarylene,

Z is one selected from the group consisting of substituted or unsubstituted C₆ to C₆₀ monocyclic or polycyclic aryl; substituted or unsubstituted C₂ to C₆₀ monocyclic or polycyclic heteroaryl; —SiR₁₁R₁₂R₁₃; and —P(═O)R₁₄R₁₅,

R₁₁ to R₁₅ are the same as or different from each other, and are each independently one selected from the group consisting of hydrogen; deuterium; linear or branched substituted or unsubstituted C₁ to C₆₀ alkyl; substituted or unsubstituted C₆ to C₆₀ monocyclic or polycyclic aryl; and substituted or unsubstituted C₂ to C₆₀ monocyclic or polycyclic heteroaryl,

m is an integer of 0 to 5,

n is an integer of 1 to 3, and

when m and n are independently integers of 2 or more, multiple L and Z are the same as or different from each other.

According to an exemplary embodiment of the present invention, in the chemical formulas 2 to 3,

R₁ is -(L)m-(Z)n,

L is one selected from the group consisting of monocyclic or polycyclic C₆ to C₆₀ arylene unsubstituted or substituted with halogen, linear or branched C₁ to C₆₀ alkyl, C₃ to C₆₀ monocyclic or polycyclic cycloalkyl, C₆ to C₆₀ monocyclic or polycyclic aryl, and C₂ to C₆₀ monocyclic or polycyclic heteroaryl; and C₂ to C₆₀ monocyclic or polycyclic heteroarylene unsubstituted or substituted with halogen, linear or branched C₁ to C₆₀ alkyl, C₃ to C₆₀ monocyclic or polycyclic cycloalkyl, C₆ to C₆₀ monocyclic or polycyclic aryl, and C₂ to C₆₀ monocyclic or polycyclic heteroaryl,

Z is one selected from the group consisting of monocyclic or polycyclic C₆ to C₆₀ aryl unsubstituted or substituted with halogen, linear or branched C₁ to C₆₀ alkyl, C₃ to C₆₀ monocyclic or polycyclic cycloalkyl, C₆ to C₆₀ monocyclic or polycyclic aryl, and C₂ to C₆₀ monocyclic or polycyclic heteroaryl; C₂ to C₆₀ monocyclic or polycyclic heteroaryl unsubstituted or substituted with halogen, linear or branched C₁ to C₆₀ alkyl, C₃ to C₆₀ monocyclic or polycyclic cycloalkyl, C₆ to C₆₀ monocyclic or polycyclic aryl, and C₂ to C₆₀ monocyclic or polycyclic heteroaryl; —SiR₁₁R₁₂R₁₃; and —P(═O)R₁₄R₁₅, and the R₁₁ to R₁₅ are the same as or different from each other, and are each independently one selected from the group consisting of hydrogen; linear or branched alkyl unsubstituted or substituted with halogen, linear or branched C₁ to C₆₀ alkyl, C₃ to C₆₀ monocyclic or polycyclic cycloalkyl, C₆ to C₆₀ monocyclic or polycyclic aryl, and C₂ to C₆₀ monocyclic or polycyclic heteroaryl; C₆ to C₆₀ monocyclic or polycyclic aryl unsubstituted or substituted with halogen, linear or branched C₁ to C₆₀ alkyl, C₃ to C₆₀ monocyclic or polycyclic cycloalkyl, C₆ to C₆₀ monocyclic or polycyclic aryl, and C₂ to C₆₀ monocyclic or polycyclic heteroaryl; and C₂ to C₆₀ monocyclic or polycyclic heteroaryl unsubstituted or substituted with halogen, linear or branched C₁ to C₆₀ alkyl, C₃ to C₆₀ monocyclic or polycyclic cycloalkyl, C₆ to C₆₀ monocyclic or polycyclic aryl, and C₂ to C₆₀ monocyclic or polycyclic heteroaryl,

m is an integer of 0 to 5,

n is an integer of 1 to 3,

when m and n are independently integers of 2 or more, multiple Ls and Zs are the same as or different from each other,

Y₁ to Y₁₂ are the same as or different from each other, and are each independently N or CR,

R is one selected from the group consisting of C₆ to C₆₀ monocyclic or polycyclic aryl unsubstituted or substituted with halogen, linear or branched C₁ to C₆₀ alkyl, C₃ to C₆₀ monocyclic or polycyclic cycloalkyl, C₆ to C₆₀ monocyclic or polycyclic aryl, and C₂ to C₆₀ monocyclic or polycyclic heteroaryl; C₂ to C₆₀ monocyclic or polycyclic heteroaryl unsubstituted or substituted with halogen, linear or branched C₁ to C₆₀ alkyl, C₃ to C₆₀ monocyclic or polycyclic cycloalkyl, C₆ to C₆₀ monocyclic or polycyclic aryl, and C₂ to C₆₀ monocyclic or polycyclic heteroaryl; —SiR₁₁R₁₂R₁₃; and —P(═O)R₁₄R₁₅, and the R₁₁ to R₁₅ are the same as or different from each other, and are each independently one selected from the group consisting of hydrogen; linear or branched C₁ to C₆₀ alkyl unsubstituted or substituted with halogen, linear or branched C₁ to C₆₀ alkyl, C₃ to C₆₀ monocyclic or polycyclic cycloalkyl, C₆ to C₆₀ monocyclic or polycyclic aryl, and C₂ to C₆₀ monocyclic or polycyclic heteroaryl; C₆ to C₆₀ monocyclic or polycyclic aryl unsubstituted or substituted with halogen, linear or branched C₁ to C₆₀ alkyl, C₃ to C₆₀ monocyclic or polycyclic cycloalkyl, C₆ to C₆₀ monocyclic or polycyclic aryl, and C₂ to C₆₀ monocyclic or polycyclic heteroaryl; and C₂ to C₆₀ monocyclic or polycyclic heteroaryl unsubstituted or substituted with halogen, linear or branched C₁ to C₆₀ alkyl, C₃ to C₆₀ monocyclic or polycyclic cycloalkyl, C₆ to C₆₀ monocyclic or polycyclic aryl, and C₂ to C₆₀ monocyclic or polycyclic heteroaryl, and

if two or more of the Y₁ to Y₁₂ are CR, multiple Rs are the same as or different from each other.

According to an exemplary embodiment of the present invention, the m is 0, or an integer of 1, 2, 3, 4, or 5, and when the m is an integer of 2 or more, Ls are the same as or different from each other.

According to an exemplary embodiment of the present invention, the n is an integer of 1, 2, or 3, and when the n is an integer of 2 or more, Zs are the same as or different from each other.

According to an exemplary embodiment of the present invention, L is C₆ to C₂₀ arylene; or C₂ to C₂₀ heteroarylene.

According to an exemplary embodiment of the present invention, L is substituted or unsubstituted phenylene; substituted or unsubstituted biphenylene; substituted or unsubstituted naphthylene; substituted or unsubstituted anthracenylene; substituted or unsubstituted pyridylene; substituted or unsubstituted pyrimidylene; or substituted or unsubstituted triazinylene.

According to an exemplary embodiment of the present invention, Z is substituted or unsubstituted C₆ to C₆₀ monocyclic to pentacyclic aryl.

According to an exemplary embodiment of the present invention, Z is substituted or unsubstituted phenyl, substituted or unsubstituted biphenyl, substituted or unsubstituted naphthyl, substituted or unsubstituted chrysenyl, substituted or unsubstituted pyrenyl, substituted or unsubstituted triphenylenyl, substituted or unsubstituted anthracenyl, substituted or unsubstituted phenanthrenyl, substituted or unsubstituted fluorenyl, substituted or unsubstituted benzo chrysenyl, or substituted or unsubstituted spirobifluorenyl.

According to an exemplary embodiment of the present invention, Z is substituted or unsubstituted phenyl, substituted or unsubstituted biphenyl, substituted or unsubstituted naphthyl, substituted or unsubstituted chrysenyl, substituted or unsubstituted pyrenyl, substituted or unsubstituted triphenylenyl, substituted or unsubstituted anthracenyl, substituted or unsubstituted phenanthrenyl, substituted or unsubstituted fluorenyl, substituted or unsubstituted benzo chrysenyl, or substituted or unsubstituted spirobifluorenyl, and the term “substituted or unsubstituted” refers to a group that may be substituted or may not be further substituted with at least one selected from halogen, linear or branched C₁ to C₆₀ alkyl, C₃ to C₆₀ monocyclic or polycyclic cycloalkyl, C₆ to C₆₀ monocyclic or polycyclic aryl, C₂ to C₆₀ monocyclic or polycyclic heteroaryl, and may be further substituted.

According to an exemplary embodiment of the present invention, Z is substituted or unsubstituted phenyl, substituted or unsubstituted biphenyl, substituted or unsubstituted naphthyl, substituted or unsubstituted chrysenyl, substituted or unsubstituted pyrenyl, substituted or unsubstituted triphenylenyl, substituted or unsubstituted anthracenyl, substituted or unsubstituted phenanthrenyl, substituted or unsubstituted fluorenyl, substituted or unsubstituted benzo chrysenyl, or substituted or unsubstituted spirobifluorenyl, and the term “substituted or unsubstituted” refers to a group that may be substituted with at least one selected from halogen atoms, methyl, phenyl, naphthyl, pyridyl, and carbazol, and may be further substituted.

According to an exemplary embodiment of the present invention, Z is substituted or unsubstituted C₂ to C₆₀ monocyclic or polycyclic heteroaryl, and the heteroaryl includes at least one selected from N, O, S, Si, and Se as a heteroatom.

According to an exemplary embodiment of the present invention, Z is substituted or unsubstituted benzimidazolyl, substituted or unsubstituted quinolyl, substituted or unsubstituted isoquinolyl, substituted or unsubstituted naphtyridyl, substituted or unsubstituted quinazolyl, substituted or unsubstituted quinoxalyl, substituted or unsubstituted cinolyl, substituted or unsubstituted benzothiazolyl, substituted or unsubstituted benzoxazolyl, substituted or unsubstituted oxadiazolyl, substituted or unsubstituted dibenzofuranyl, substituted or unsubstituted dibenzothiophenyl, substituted or unsubstituted pyridyl, substituted or unsubstituted pyrimidyl, substituted or unsubstituted triazinyl, substituted or unsubstituted pyrazolophthalazinyl, or substituted or unsubstituted carbazolyl.

According to an exemplary embodiment of the present invention, Z is substituted or unsubstituted benzimidazolyl, substituted or unsubstituted quinolyl, substituted or unsubstituted isoquinolyl, substituted or unsubstituted naphtyridyl, substituted or unsubstituted quinazolyl, substituted or unsubstituted quinoxalyl, substituted or unsubstituted cinolyl, substituted or unsubstituted benzothiazolyl, substituted or unsubstituted benzoxazolyl, substituted or unsubstituted oxadiazolyl, substituted or unsubstituted dibenzofuranyl, substituted or unsubstituted dibenzothiophenyl, substituted or unsubstituted pyridyl, substituted or unsubstituted pyrimidyl, substituted or unsubstituted triazinyl, substituted or unsubstituted pyrazolophthalazinyl, or substituted or unsubstituted carbazolyl, and the term “substituted or unsubstituted” refers to a group that may be substituted or may not be further substituted with at least one selected from halogen atoms, monocyclic or polycyclic C₃ to C₆₀ cycloalkyl, monocyclic or polycyclic C₆ to C₆₀ aryl, monocyclic or polycyclic C₂ to C₆₀ heteroaryl, and SiR₁₁R₁₂R₁₃, and may be further substituted, and

R₁₁ to R₁₃ are the same as or different from each other, and are each independently linear or branched C₁ to C₆₀ alkyl; C₆ to C₆₀ monocyclic or polycyclic aryl; or C₂ to C₆₀ monocyclic or polycyclic heteroaryl.

According to an exemplary embodiment of the present invention, Z is substituted or unsubstituted benzimidazolyl, substituted or unsubstituted quinolyl, substituted or unsubstituted isoquinolyl, substituted or unsubstituted naphtyridyl, substituted or unsubstituted quinazolyl, substituted or unsubstituted quinoxalyl, substituted or unsubstituted cinolyl, substituted or unsubstituted benzothiazolyl, substituted or unsubstituted benzoxazolyl, substituted or unsubstituted oxadiazolyl, substituted or unsubstituted dibenzofuranyl, substituted or unsubstituted dibenzothiophenyl, substituted or unsubstituted pyridyl, substituted or unsubstituted pyrimidyl, substituted or unsubstituted triazinyl, substituted or unsubstituted pyrazolophthalazinyl, or substituted or unsubstituted carbazolyl, and the term “substituted or unsubstituted” refers to a group that may be substituted or may not be further substituted with at least one selected from halogen atoms, cyclohexyl, phenyl, naphthyl, triphenylenyl, pyridyl, and Si(CH₃)₃, and may be further substituted.

According to an exemplary embodiment of the present invention, Z is

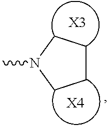

and the X3 and X4 are substituted or unsubstituted C₆ to C₆₀ monocyclic or polycyclic aromatic hydrocarbon rings; or substituted or unsubstituted C₂ to C₆₀ monocyclic or polycyclic aromatic hetero rings.

According to an exemplary embodiment of the present invention,

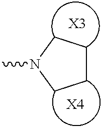

is represented by any one of the following structural formulas.

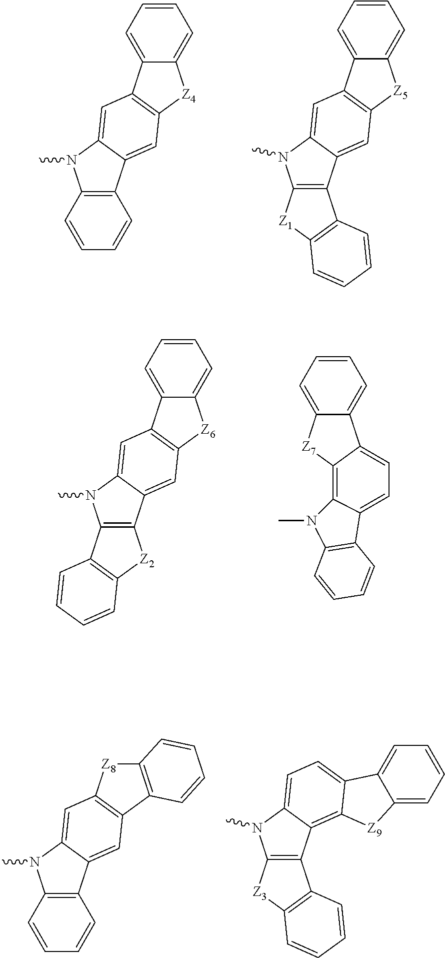

In the structural formulas, Z₁ to Z₃ are the same as or different from each other, and are each independently S or O,

Z₄ to Z₉ are the same as or different from each other, and are each independently CR′R″, NR′, S, or O,

R′ and R″ are the same as or different from each other, and are each independently hydrogen; linear or branched substituted or unsubstituted C₁ to C₆₀ alkyl; or substituted or unsubstituted C₆ to C₆₀ monocyclic or polycyclic aryl.

According to an exemplary embodiment of the present invention, the R′ and R″ are the same as or different from each other, and are independently hydrogen, methyl, phenyl, or naphthyl.

According to an exemplary embodiment of the present invention, Z is —P(═O)R₁₄R₁₅, and R₁₄ and R₁₅ are the same as or different from each other, and are independently one selected from the group consisting of C₆ to C₆₀ monocyclic or polycyclic aryl; and C₂ to C₆₀ monocyclic or polycyclic heteroaryl.

According to an exemplary embodiment of the present invention, Z is —P(═O)R₁₄R₁₅, and R₁₄ and R₁₅ are the same as or different from each other, and are independently C₆ to C₆₀ monocyclic or polycyclic aryl.

According to an exemplary embodiment of the present invention, Z is —P(═O)R₁₄R₁₅, and R₁₄ and R₁₅ are the same as or different from each other, and are independently phenyl, biphenyl, or naphthyl.

According to an exemplary embodiment of the present invention, L is selected from the group consisting of substituted or unsubstituted C₆ to C₆₀ monocyclic or polycyclic arylene and substituted or unsubstituted C₂ to C₆₀ monocyclic or polycyclic heteroarylene, and Z is selected from the group consisting of substituted or unsubstituted C₆ to C₆₀ monocyclic or polycyclic aryl.

According to an exemplary embodiment of the present invention, L may be selected from the group consisting of phenylene, biphenylene, naphthylene, anthracenylene, phenanthrenylene, triphenylenylene, chrysenylene, benzo chrysenylene, fluorenylene, pyrenylene, spirobifluorenylene, pyridylene, imidazopyridylene, pyrimidylene, triazinylene, carbazolylene, benzimidazolylene, benzocarbazolylene, dibenzocarbazolylene, quinolinylene, isoquinolinylene, quinazolinylene, pyrazoloquinazolinylene, imidazoquinazolinylene, thiazolinylene, benzothiazolinylene, phenanthrolinylene, phenanthridinylene, dibenzo acridinylene, xylolylene, benzoxylolylene, dibenzoxylolylene, benzoxazolylene, naphthyridinylene, oxadiazolylene, dibenzofuranylene, dibenzothiophenylene, dibenzophenanthridinylene, and spirobidibenzoxylolylene, and may be further substituted with halogen; C₁ to C₁₀ alkyl; C₃ to C₃₀ cycloalkyl; C₆ to C₃₀ aryl; and C₂ to C₃₀ heteroaryl, and

Z may be selected from the group consisting of phenyl, biphenyl, naphthyl, anthracenyl, phenanthrenyl, triphenylenyl, chrysenyl, benzo chrysenyl, fluorenyl, pyrenyl, and spirobifluorenyl, and may be further substituted with halogen atoms; C₁ to C₁₀ alkyl; C₃ to C₃₀ cycloalkyl; C₆ to C₃₀ aryl; and C₂ to C₃₀ heteroaryl.

According to an exemplary embodiment of the present invention, L may be selected from the group consisting of phenylene, biphenylene, naphthylene, anthracenylene, phenanthrenylene, triphenylenylene, chrysenylene, benzo chrysenylene, fluorenylene, pyrenylene, spirobifluorenylene, pyridylene, imidazopyridylene, pyrimidylene, triazinylene, carbazolylene, benzimidazolylene, benzocarbazolylene, dibenzocarbazolylene, quinolinylene, isoquinolinylene, quinazolinylene, pyrazoloquinazolinylene, imidazoquinazolinylene, thiazolinylene, benzothiazolinylene, phenanthrolinylene, phenanthridinylene, dibenzo acridinylene, xylolylene, benzoxylolylene, dibenzoxylolylene, benzoxazolylene, naphthyridinylene, oxadiazolylene, dibenzofuranylene, dibenzothiophenylene, dibenzophenanthridinylene, and spirobidibenzoxylolylene, and may be further substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl, and

Z may be selected from the group consisting of phenyl, biphenyl, naphthyl, anthracenyl, phenanthrenyl, triphenylenyl, chrysenyl, benzo chrysenyl, fluorenyl, pyrenyl, and spirobifluorenyl, and may be further substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl.

According to an exemplary embodiment of the present invention, L is selected from the group consisting of substituted or unsubstituted C₆ to C₆₀ monocyclic or polycyclic arylene and substituted or unsubstituted C₂ to C₆₀ monocyclic or polycyclic heteroarylene, and Z is selected from the group consisting of substituted or unsubstituted C₂ to C₆₀ monocyclic or polycyclic heteroaryl.

According to an exemplary embodiment of the present invention, L may be selected from the group consisting of phenylene, biphenylene, naphthylene, anthracenylene, phenanthrenylene, triphenylenylene, chrysenylene, benzo chrysenylene, fluorenylene, pyrenylene, spirobifluorenylene, pyridylene, imidazopyridylene, pyrimidylene, triazinylene, carbazolylene, benzimidazolylene, benzocarbazolylene, dibenzocarbazolylene, quinolinylene, isoquinolinylene, quinazolinylene, pyrazoloquinazolinylene, imidazoquinazolinylene, thiazolinylene, benzothiazolinylene, phenanthrolinylene, phenanthridinylene, dibenzo acridinylene, xylolylene, benzoxylolylene, dibenzoxylolylene, benzoxazolylene, naphthyridinylene, oxadiazolylene, dibenzofuranylene, dibenzothiophenylene, dibenzophenanthridinylene, and spirobidibenzoxylolylene, and may be further substituted with halogen; C₁ to C₁₀ alkyl; C₃ to C₃₀ cycloalkyl; C₆ to C₃₀ aryl; and C₂ to C₃₀ heteroaryl, and

Z may be selected from the group consisting of pyridyl, imidazol pyridyl, pyrimidyl, triazinyl, carbazolyl, benzimidazolyl, benzocarbazolyl, dibenzocarbazolyl, quinolinyl, isoquinolinyl, quinazolinyl, pyrazoloquinazolinyl, imidazoquinazolinyl, thiazolinyl, benzothiazolinyl, phenanthrolinyl, phenanthridinyl, dibenzo acridinyl, xylolyl, benzoxylolyl, dibenzoxylolyl, benzoxazolyl, naphthyridinyl, oxadiazolyl, dibenzofuranyl, dibenzothiophenyl, dibenzophenanthridinyl, and spirobidibenzoxylolyl, and may be further substituted with halogen atoms; C₁ to C₁₀ alkyl; C₃ to C₃₀ cycloalkyl; C₆ to C₃₀ aryl; and C₂ to C₃₀ heteroaryl.

According to an exemplary embodiment of the present invention, L may be selected from the group consisting of phenylene, biphenylene, naphthylene, anthracenylene, phenanthrenylene, triphenylenylene, chrysenylene, benzo chrysenylene, fluorenylene, pyrenylene, spirobifluorenylene, pyridylene, imidazopyridylene, pyrimidylene, triazinylene, carbazolylene, benzimidazolylene, benzocarbazolylene, dibenzocarbazolylene, quinolinylene, isoquinolinylene, quinazolinylene, pyrazoloquinazolinylene, imidazoquinazolinylene, thiazolinylene, benzothiazolinylene, phenanthrolinylene, phenanthridinylene, dibenzo acridinylene, xylolylene, benzoxylolylene, dibenzoxylolylene, benzoxazolylene, naphthyridinylene, oxadiazolylene, dibenzofuranylene, dibenzothiophenylene, dibenzophenanthridinylene, and spirobidibenzoxylolylene, and may be further substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl, and

Z may be selected from the group consisting of pyridyl, imidazol pyridyl, pyrimidyl, triazinyl, carbazolyl, benzimidazolyl, benzocarbazolyl, dibenzocarbazolyl, quinolinyl, isoquinolinyl, quinazolinyl, pyrazoloquinazolinyl, imidazoquinazolinyl, thiazolinyl, benzothiazolinyl, phenanthrolinyl, phenanthridinyl, dibenzo acridinyl, xylolyl, benzoxylolyl, dibenzoxylolyl, benzoxazolyl, naphthyridinyl, oxadiazolyl, dibenzofuranyle, dibenzothiophenyl, dibenzophenanthridinyl, and spirobidibenzoxylolyl, and may be further substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl.

According to an exemplary embodiment of the present invention, L is substituted or unsubstituted C₆ to C₆₀ monocyclic or polycyclic arylene, Z is —SiR₁₁R₁₂R₁₃, and the R₁₁ to R₁₃ are the same as or different from each other, and are each independently one selected from the group consisting of hydrogen atoms; deuterium; linear or branched substituted or unsubstituted C₁ to C₆₀ alkyl; substituted or unsubstituted C₆ to C₆₀ monocyclic or polycyclic aryl; and substituted or unsubstituted C₂ to C₆₀ monocyclic or polycyclic heteroaryl.

According to an exemplary embodiment of the present invention, L may be selected from the group consisting of phenylene, biphenylene, naphthylene, anthracenylene, phenanthrenylene, triphenylenylene, chrysenylene, benzo chrysenylene, fluorenylene, pyrenylene, and spirobifluorenylene, and may be further substituted with halogen; C₁ to C₁₀ alkyl; C₃ to C₃₀ cycloalkyl; C₆ to C₃₀ aryl; and C₂ to C₃₀ heteroaryl,

Z is —SiR₁₁R₁₂R₁₃, and the R₁₁ to R₁₃ are the same as or different from each other, and are each independently one selected from the group consisting of phenyl, biphenyl, naphthyl, and anthracenyl, and may be further substituted with halogen atoms; C₁ to C₁₀ alkyl; C₃ to C₃₀ cycloalkyl; C₆ to C₃₀ aryl; and C₂ to C₃₀ heteroaryl.

According to an exemplary embodiment of the present invention, L may be selected from the group consisting of phenylene, biphenylene, naphthylene, anthracenylene, phenanthrenylene, triphenylenylene, chrysenylene, benzo chrysenylene, fluorenylene, pyrenylene, and spirobifluorenylene, and may be further substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl,

the Z is —SiR₁₁R₁₂R₁₃, and the R₁₁ to R₁₃ are the same as or different from each other, and are each independently one selected from the group consisting of phenyl, biphenyl, naphthyl, and anthracenyl, and may be further substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl.

According to an exemplary embodiment of the present invention, L is substituted or unsubstituted C₆ to C₆₀ monocyclic or polycyclic arylene, Z is —P(═O)R₁₄R₁₅, and the R₁₄ and R₁₅ are the same as or different from each other, and are each independently one selected from the group consisting of hydrogen; deuterium; linear or branched substituted or unsubstituted C₁ to C₆₀ alkyl; substituted or unsubstituted C₆ to C₆₀ monocyclic or polycyclic aryl; and substituted or unsubstituted C₂ to C₆₀ monocyclic or polycyclic heteroaryl.

According to an exemplary embodiment of the present invention, L may be selected from the group consisting of phenylene, biphenylene, naphthylene, anthracenylene, phenanthrenylene, triphenylenylene, chrysenylene, benzo chrysenylene, fluorenylene, pyrenylene, and spirobifluorenylene, and may be further substituted with halogen; C₁ to C₁₀ alkyl; C₃ to C₃₀ cycloalkyl; C₆ to C₃₀ aryl; and C₂ to C₃₀ heteroaryl,

Z is —P(═O)R₁₄R₁₅, and the R₁₄ and R₁₅ are the same as or different from each other, and are each independently one selected from the group consisting of phenyl, biphenyl, naphthyl, and anthracenyl, and may be further substituted with halogen; C₁ to C₁₀ alkyl; C₃ to C₃₀ cycloalkyl; C₆ to C₃₀ aryl; and C₂ to C₃₀ heteroaryl.

According to an exemplary embodiment of the present invention, L may be selected from the group consisting of phenylene, biphenylene, naphthylene, anthracenylene, phenanthrenylene, triphenylenylene, chrysenylene, benzo chrysenylene, fluorenylene, pyrenylene, and spirobifluorenylene, and may be further substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl,

Z is —P(═O)R₁₄R₁₅, and the R₁₄ and R₁₅ which are identical to or different from each other, are each independently one selected from the group consisting of phenyl, biphenyl, naphthyl, and anthracenyl, and may be further substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl.

According to an exemplary embodiment of the present invention, L is monocyclic or polycyclic substituted or unsubstituted C₆ to C₆₀ monocyclic or polycyclic arylene, Z is monocyclic or polycyclic substituted or unsubstituted C₂ to C₆₀ N-containing monocyclic or polycyclic heteroaryl.

According to an exemplary embodiment of the present invention, L may be selected from the group consisting of phenylene, biphenylene, naphthylene, anthracenylene, phenanthrenylene, triphenylenylene, chrysenylene, benzo chrysenylene, fluorenylene, pyrenylene, and spirobifluorenylene, and may be further substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl, and

Z may be pyridyl unsubstituted or substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl.

According to an exemplary embodiment of the present invention, L may be selected from the group consisting of phenylene, biphenylene, naphthylene, anthracenylene, phenanthrenylene, triphenylenylene, chrysenylene, benzo chrysenylene, fluorenylene, pyrenylene, and spirobifluorenylene, and may be further substituted with halogen; C₁ to C₁₀ alkyl; C₃ to C₃₀ cycloalkyl; C₆ to C₃₀ aryl; and C₂ to C₃₀ heteroaryl, and

Z may be pyrimidyl unsubstituted or substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl.

According to an exemplary embodiment of the present invention, L may be selected from the group consisting of phenylene, biphenylene, naphthylene, anthracenylene, phenanthrenylene, triphenylenylene, chrysenylene, benzo chrysenylene, fluorenylene, pyrenylene, and spirobifluorenylene, and may be further substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl, and

Z may be triazinyl substituted or unsubstituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl.

According to an exemplary embodiment of the present invention, L may be selected from the group consisting of phenylene, biphenylene, naphthylene, anthracenylene, phenanthrenylene, triphenylenylene, chrysenylene, benzo chrysenylene, fluorenylene, pyrenylene, and spirobifluorenylene, and may be further substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl, and

Z may be carbazolyl unsubstituted or substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl.

According to an exemplary embodiment of the present invention, L may be selected from the group consisting of phenylene, biphenylene, naphthylene, anthracenylene, phenanthrenylene, triphenylenylene, chrysenylene, benzo chrysenylene, fluorenylene, pyrenylene, and spirobifluorenylene, and may be further substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl, and

the Z may be quinolinyl unsubstituted or substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl.

According to an exemplary embodiment of the present invention, L may be selected from the group consisting of phenylene, biphenylene, naphthylene, anthracenylene, phenanthrenylene, triphenylenylene, chrysenylene, benzo chrysenylene, fluorenylene, pyrenylene, and spirobifluorenylene, and may be further substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl, and

Z may be quinazolinyl unsubstituted or substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl.

According to an exemplary embodiment of the present invention, L may be selected from the group consisting of phenylene, biphenylene, naphthylene, anthracenylene, phenanthrenylene, triphenylenylene, chrysenylene, benzo chrysenylene, fluorenylene, pyrenylene, and spirobifluorenylene, and may be further substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl, and

Z may be pyrazoloquinazolinyl unsubstituted or substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl.

According to an exemplary embodiment of the present invention, the L may be selected from the group consisting of phenylene, biphenylene, naphthylene, anthracenylene, phenanthrenylene, triphenylenylene, chrysenylene, benzo chrysenylene, fluorenylene, pyrenylene, and spirobifluorenylene, and may be further substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl, and

Z may be phenanthrolinyl unsubstituted or substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl.

According to an exemplary embodiment of the present invention, L may be selected from the group consisting of phenylene, biphenylene, naphthylene, anthracenylene, phenanthrenylene, triphenylenylene, chrysenylene, benzo chrysenylene, fluorenylene, pyrenylene, and spirobifluorenylene, and may be further substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl, and

Z may be benzimidazolyl unsubstituted or substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl.

According to an exemplary embodiment of the present invention, L may be selected from the group consisting of phenylene, biphenylene, naphthylene, anthracenylene, phenanthrenylene, triphenylenylene, chrysenylene, benzo chrysenylene, fluorenylene, pyrenylene, and spirobifluorenylene, and may be further substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl, and

Z may be benzothiazolyl unsubstituted or substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl.

According to an exemplary embodiment of the present invention, L may be selected from the group consisting of phenylene, biphenylene, naphthylene, anthracenylene, phenanthrenylene, triphenylenylene, chrysenylene, benzo chrysenylene, fluorenylene, pyrenylene, and spirobifluorenylene, and may be further substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl, and

Z may be benzoxazolyl unsubstituted or substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl.

According to an exemplary embodiment of the present invention, L may be selected from the group consisting of phenylene, biphenylene, naphthylene, anthracenylene, phenanthrenylene, triphenylenylene, chrysenylene, benzo chrysenylene, fluorenylene, pyrenylene, and spirobifluorenylene, and may be further substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl, and

Z may be oxadiazolyl unsubstituted or substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl.

According to an exemplary embodiment of the present invention, L may be selected from the group consisting of phenylene, biphenylene, naphthylene, anthracenylene, phenanthrenylene, triphenylenylene, chrysenylene, benzo chrysenylene, fluorenylene, pyrenylene, and spirobifluorenylene, and may be further substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl, and

Z may be naphthyridinyl unsubstituted or substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl.

According to an exemplary embodiment of the present invention, L may be selected from the group consisting of phenylene, biphenylene, naphthylene, anthracenylene, phenanthrenylene, triphenylenylene, chrysenylene, benzo chrysenylene, fluorenylene, pyrenylene, and spirobifluorenylene, and may be further substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl, and

Z may be phenanthrolinyl unsubstituted or substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl.

According to an exemplary embodiment of the present invention, L may be selected from the group consisting of phenylene, biphenylene, naphthylene, anthracenylene, phenanthrenylene, triphenylenylene, chrysenylene, benzo chrysenylene, fluorenylene, pyrenylene, and spirobifluorenylene, and may be further substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl, and

Z may be dibenzophenanthridinyl unsubstituted or substituted with at least one substituent selected from the group consisting of fluorine, methyl, ethyl, propyl, cyclopentyl, cyclohexyl, phenyl, biphenyl, naphthyl, anthracenyl, phenanthrenyl, pyridyl, and pyrimidyl.

According to an exemplary embodiment of the present invention, the chemical formula 1 is represented by the following chemical formula 4 or 5.

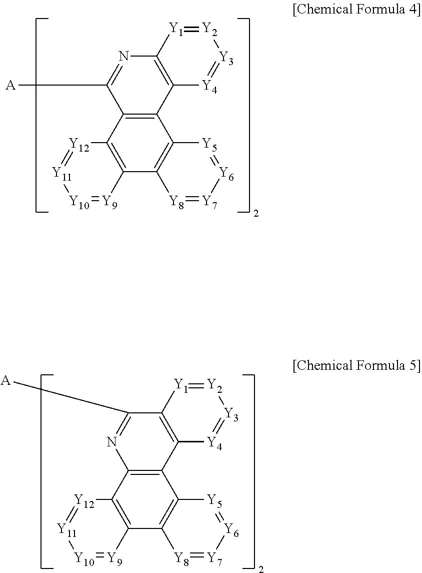

wherein in the chemical formulas 4 and 5,

A is directly bonded; or selected from the group consisting of linear or branched substituted or unsubstituted C₂ to C₆₀ alkylene, linear or branched substituted or unsubstituted C₂ to C₆₀ alkenylene, linear or branched substituted or unsubstituted C₂ to C₆₀ alkynylene, substituted or unsubstituted C₃ to C₆₀ monocyclic or polycyclic cycloalkylene, substituted or unsubstituted C₂ to C₆₀ monocyclic or polycyclic heterocycloalkylene, substituted or unsubstituted C₆ to C₆₀ monocyclic or polycyclic arylene, and substituted or unsubstituted C₂ to C₆₀ monocyclic or polycyclic heteroarylene, and

Y₁ to Y₁₂ are the same as defined in the chemical formula 1.

The chemical formulas 4 and 5 mean dimer structures, and the A means a linkage of a dimer.

According to an exemplary embodiment of the present invention, A is directly bonded; or selected from the group consisting of substituted or unsubstituted C₆ to C₆₀ monocyclic or polycyclic arylene, and substituted or unsubstituted C₂ to C₆₀ monocyclic or polycyclic heteroarylene.

According to an exemplary embodiment of the present invention, A is directly bonded or C₆ to C₆₀ monocyclic or polycyclic arylene.

According to an exemplary embodiment of the present invention, A is directly bonded or phenylene, biphenylene, naphthylene, or anthracenylene.

According to an exemplary embodiment of the present application, Formula 1 is represented by the following Formula 9.

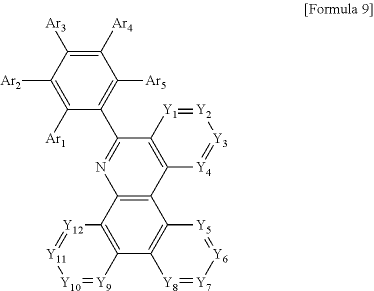

In Formula 9,

at least two of Ar₁ to Ar₅ are monocyclic or polycyclic substituted or unsubstituted C₆ to C₆₀ aryl; or monocyclic or polycyclic substituted or unsubstituted C₂ to C₆₀ heteroaryl, and the others are each independently hydrogen; deuterium; halogen; straight-chained or branched substituted or unsubstituted C₁ to C₆₀ alkyl; monocyclic or polycyclic substituted or unsubstituted C₆ to C₆₀ aryl; or monocyclic or polycyclic substituted or unsubstituted C₂ to C₆₀ heteroaryl,

Y₁ to Y₁₂ are the same as or different from each other, and are each independently N or CR,

R's are the same as or different from each other, and are each independently selected from the group consisting of hydrogen; deuterium; halogen; straight-chained or branched substituted or unsubstituted C₁ to C₆₀ alkyl; straight-chained or branched substituted or unsubstituted C₂ to C₆₀ alkenyl; straight-chained or branched substituted or unsubstituted C₂ to C₆₀ alkynyl; straight-chained or branched substituted or unsubstituted C₁ to C₆₀ alkoxy; monocyclic or polycyclic substituted or unsubstituted C₃ to C₆₀ cyclocalkyl; monocyclic or polycyclic substituted or unsubstituted C₂ to C₆₀ heterocycloalkyl; monocyclic or polycyclic substituted or unsubstituted C₆ to C₆₀ aryl; monocyclic or polycyclic substituted or unsubstituted C₂ to C₆₀ heteroaryl; —SiR₁₁R₁₂R₁₃; —P(═O)R₁₄R₁₅; and C₁ to C₂₀ alkyl, monocyclic or polycyclic substituted or unsubstituted C₆ to C₆₀ aryl, or an amine which is unsubstituted or substituted with monocyclic or polycyclic substituted or unsubstituted C₂ to C₆₀ heteroaryl, or at least two adjacent R's combine with each other to form a monocyclic or polycyclic substituted or unsubstituted aliphatic or aromatic hydrocarbon ring, and

R₁₁ to R₁₅ are the same as or different from each other, and may be each independently selected from the group consisting of straight-chained or branched substituted or unsubstituted C₁ to C₆₀ alkyl; monocyclic or polycyclic substituted or unsubstituted C₃ to C₆₀ cycloalkyl; monocyclic or polycyclic substituted or unsubstituted C₆ to C₆₀ aryl; and monocyclic or polycyclic substituted or unsubstituted C₂ to C₆₀ heteroaryl.

According to an exemplary embodiment of the present application, Formula 9 is represented by the following Formula 10.

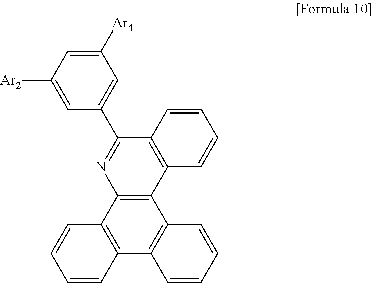

In Formula 10, Ar₂ and Ar₄ are each independently monocyclic or polycyclic substituted or unsubstituted C₆ to C₆₀ aryl; or monocyclic or polycyclic substituted or unsubstituted C₂ to C₆₀ heteroaryl.

According to an exemplary embodiment of the present application, Ar₂ and Ar₄ are each independently selected from the group consisting of a phenyl group which is unsubstituted or substituted with benzoimidazole, a naphthyl group, a phenanthrene group, a dibenzofuran group, a dibenzothiophene group, a carbazole group, a phenanthroline group, and a spirobifluorene group.

According to an exemplary embodiment of the present application, Ar₂ and Ar₄ are the same as each other.

According to an exemplary embodiment of the present invention, the chemical formula 1 may be selected from the following compounds:

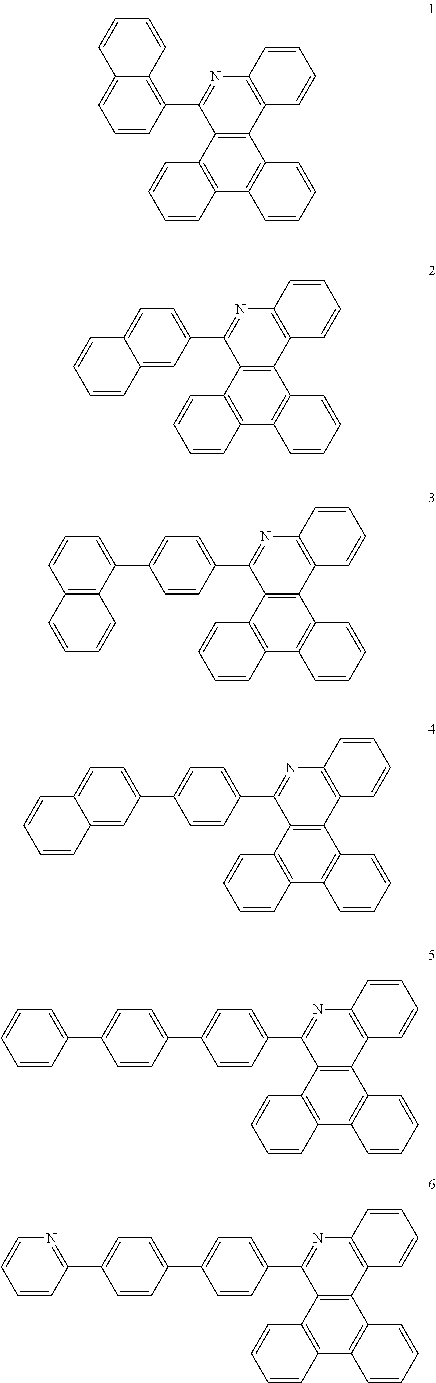 [SMILES: c1ccc2c(-c3nc4ccccc4c4c5ccccc5c5ccccc5c34)cccc2c1 ; c1ccc2cc(-c3nc4ccccc4c4c5ccccc5c5ccccc5c34)ccc2c1 ; c1ccc2c(-c3ccc(-c4nc5ccccc5c5c6ccccc6c6ccccc6c45)cc3)cccc2c1 ; c1ccc2cc(-c3ccc(-c4nc5ccccc5c5c6ccccc6c6ccccc6c45)cc3)ccc2c1 ; c1ccc(-c2ccc(-c3ccc(-c4nc5ccccc5c5c6ccccc6c6ccccc6c45)cc3)cc2)cc1 ; c1ccc(-c2ccc(-c3ccc(-c4nc5ccccc5c5c6ccccc6c6ccccc6c45)cc3)cc2)nc1]

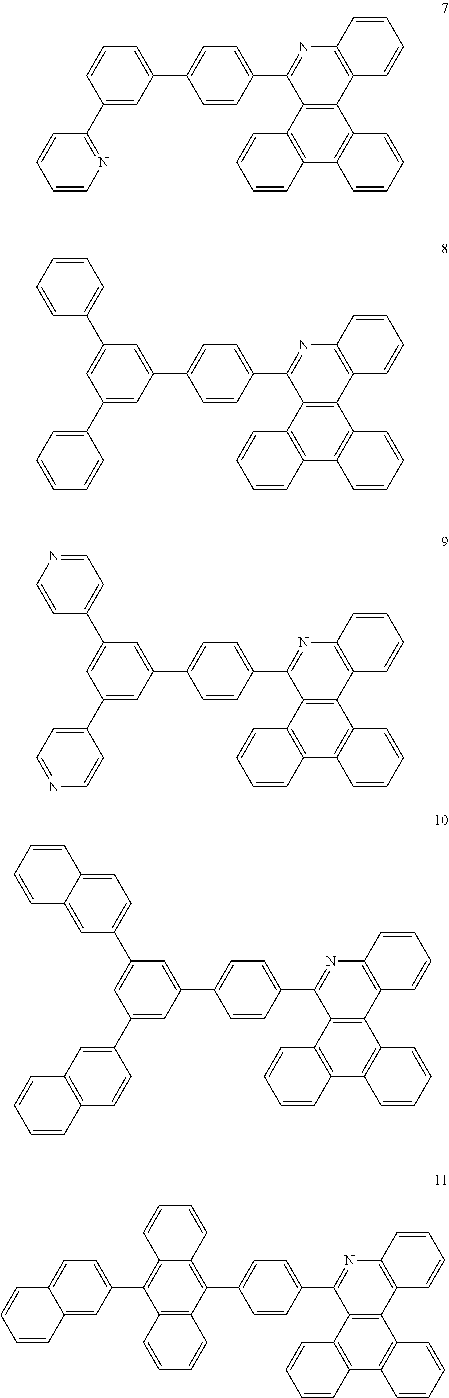 [SMILES: c1ccc(-c2cccc(-c3ccc(-c4nc5ccccc5c5c6ccccc6c6ccccc6c45)cc3)c2)nc1 ; c1ccc(-c2cc(-c3ccccc3)cc(-c3ccc(-c4nc5ccccc5c5c6ccccc6c6ccccc6c45)cc3)c2)cc1 ; c1ccc2c(c1)nc(-c1ccc(-c3cc(-c4ccncc4)cc(-c4ccncc4)c3)cc1)c1c3ccccc3c3ccccc3c21 ; c1ccc2cc(-c3cc(-c4ccc(-c5nc6ccccc6c6c7ccccc7c7ccccc7c56)cc4)cc(-c4ccc5ccccc5c4)c3)ccc2c1 ; c1ccc2cc(-c3c4ccccc4c(-c4ccc(-c5nc6ccccc6c6c7ccccc7c7ccccc7c56)cc4)c4ccccc34)ccc2c1]

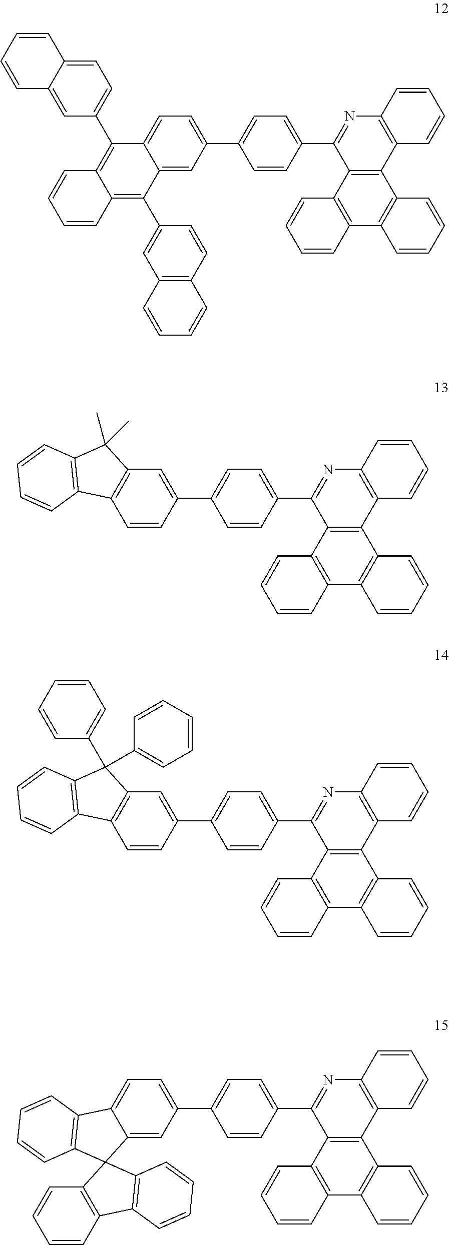 [SMILES: c1ccc2cc(-c3c4ccccc4c(-c4ccc5ccccc5c4)c4cc(-c5ccc(-c6nc7ccccc7c7c8ccccc8c8ccccc8c67)cc5)ccc34)ccc2c1 ; CC1(C)c2ccccc2-c2ccc(-c3ccc(-c4nc5ccccc5c5c6ccccc6c6ccccc6c45)cc3)cc21 ; c1ccc(C2(c3ccccc3)c3ccccc3-c3ccc(-c4ccc(-c5nc6ccccc6c6c7ccccc7c7ccccc7c56)cc4)cc32)cc1 ; c1ccc2c(c1)-c1ccccc1C21c2ccccc2-c2ccc(-c3ccc(-c4nc5ccccc5c5c6ccccc6c6ccccc6c45)cc3)cc21]

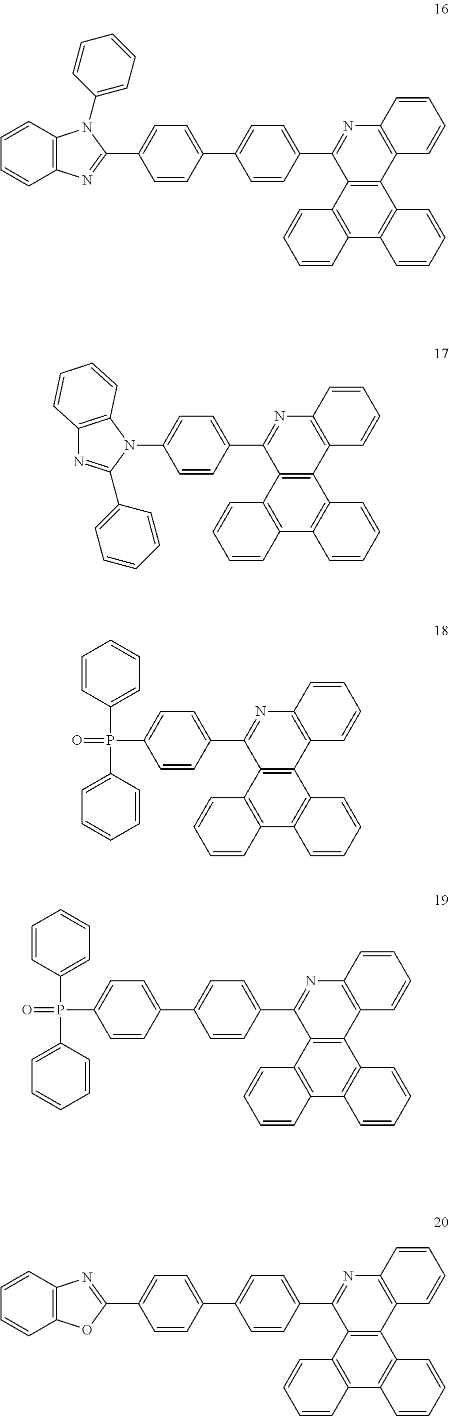 [SMILES: c1ccc(-n2c(-c3ccc(-c4ccc(-c5nc6ccccc6c6c7ccccc7c7ccccc7c56)cc4)cc3)nc3ccccc32)cc1 ; c1ccc(-c2nc3ccccc3n2-c2ccc(-c3nc4ccccc4c4c5ccccc5c5ccccc5c34)cc2)cc1 ; O=P(c1ccccc1)(c1ccccc1)c1ccc(-c2nc3ccccc3c3c4ccccc4c4ccccc4c23)cc1 ; O=P(c1ccccc1)(c1ccccc1)c1ccc(-c2ccc(-c3nc4ccccc4c4c5ccccc5c5ccccc5c34)cc2)cc1 ; c1ccc2oc(-c3ccc(-c4ccc(-c5nc6ccccc6c6c7ccccc7c7ccccc7c56)cc4)cc3)nc2c1]

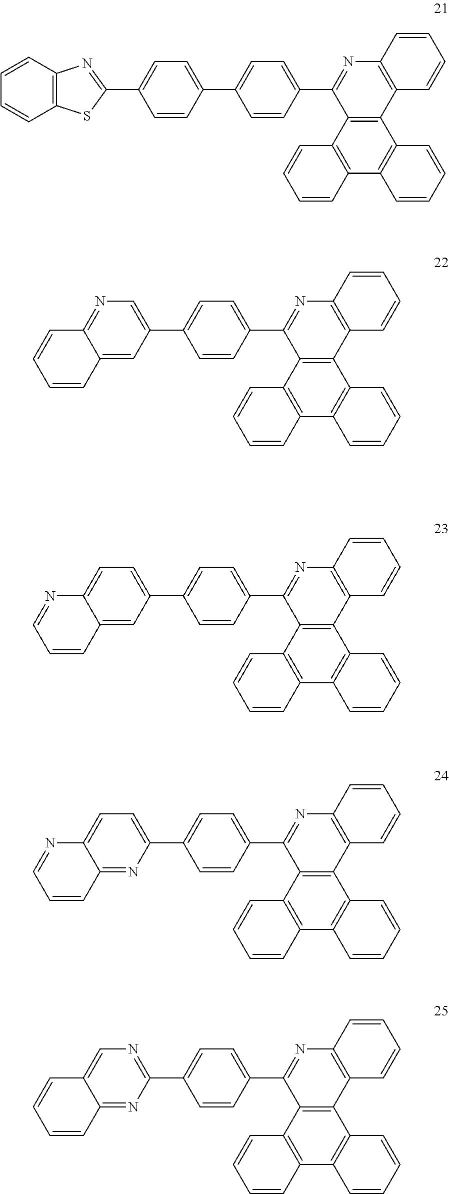 [SMILES: c1ccc2sc(-c3ccc(-c4ccc(-c5nc6ccccc6c6c7ccccc7c7ccccc7c56)cc4)cc3)nc2c1 ; c1ccc2ncc(-c3ccc(-c4nc5ccccc5c5c6ccccc6c6ccccc6c45)cc3)cc2c1 ; c1cnc2ccc(-c3ccc(-c4nc5ccccc5c5c6ccccc6c6ccccc6c45)cc3)cc2c1 ; c1cnc2ccc(-c3ccc(-c4nc5ccccc5c5c6ccccc6c6ccccc6c45)cc3)nc2c1 ; c1ccc2nc(-c3ccc(-c4nc5ccccc5c5c6ccccc6c6ccccc6c45)cc3)ncc2c1]

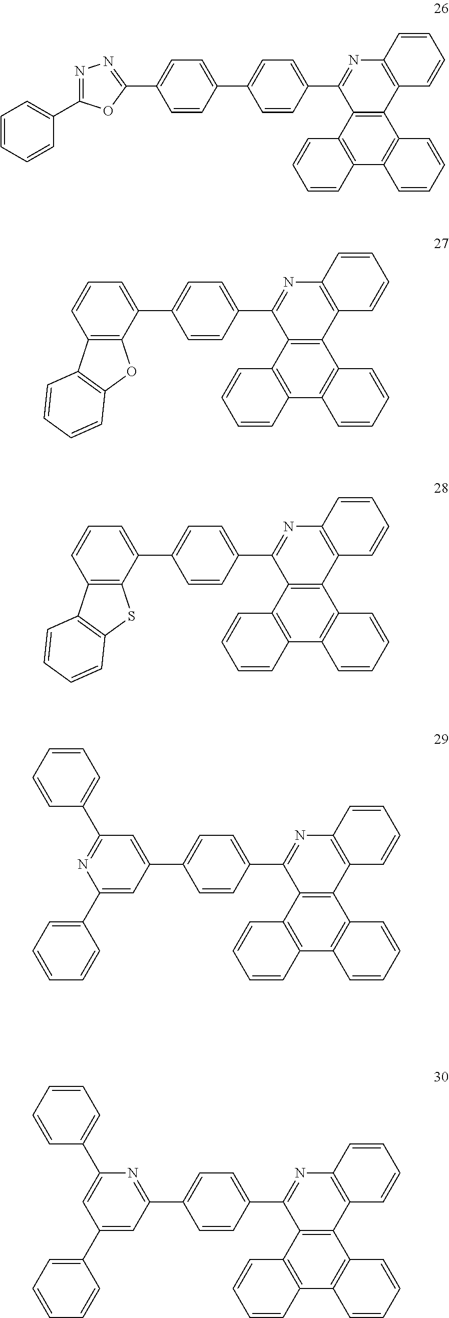 [SMILES: c1ccc(-c2nnc(-c3ccc(-c4ccc(-c5nc6ccccc6c6c7ccccc7c7ccccc7c56)cc4)cc3)o2)cc1 ; c1ccc2c(c1)nc(-c1ccc(-c3cccc4c3oc3ccccc34)cc1)c1c3ccccc3c3ccccc3c21 ; c1ccc2c(c1)nc(-c1ccc(-c3cccc4c3sc3ccccc34)cc1)c1c3ccccc3c3ccccc3c21 ; c1ccc(-c2cc(-c3ccc(-c4nc5ccccc5c5c6ccccc6c6ccccc6c45)cc3)cc(-c3ccccc3)n2)cc1 ; c1ccc(-c2cc(-c3ccccc3)nc(-c3ccc(-c4nc5ccccc5c5c6ccccc6c6ccccc6c45)cc3)c2)cc1]

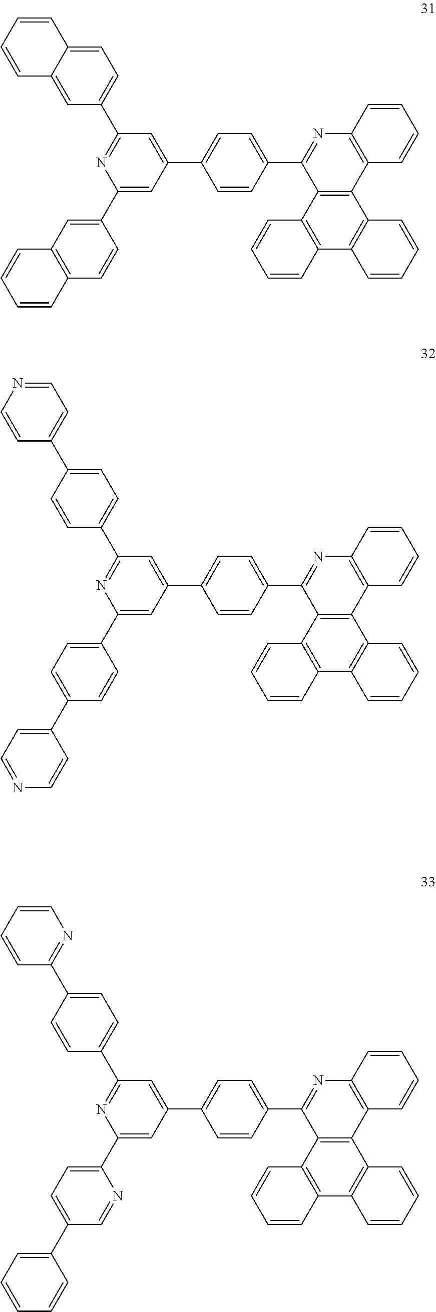 [SMILES: c1ccc2cc(-c3cc(-c4ccc(-c5nc6ccccc6c6c7ccccc7c7ccccc7c56)cc4)cc(-c4ccc5ccccc5c4)n3)ccc2c1 ; c1ccc2c(c1)nc(-c1ccc(-c3cc(-c4ccc(-c5ccncc5)cc4)nc(-c4ccc(-c5ccncc5)cc4)c3)cc1)c1c3ccccc3c3ccccc3c21 ; c1ccc(-c2ccc(-c3cc(-c4ccc(-c5nc6ccccc6c6c7ccccc7c7ccccc7c56)cc4)cc(-c4ccc(-c5ccccn5)cc4)n3)nc2)cc1]

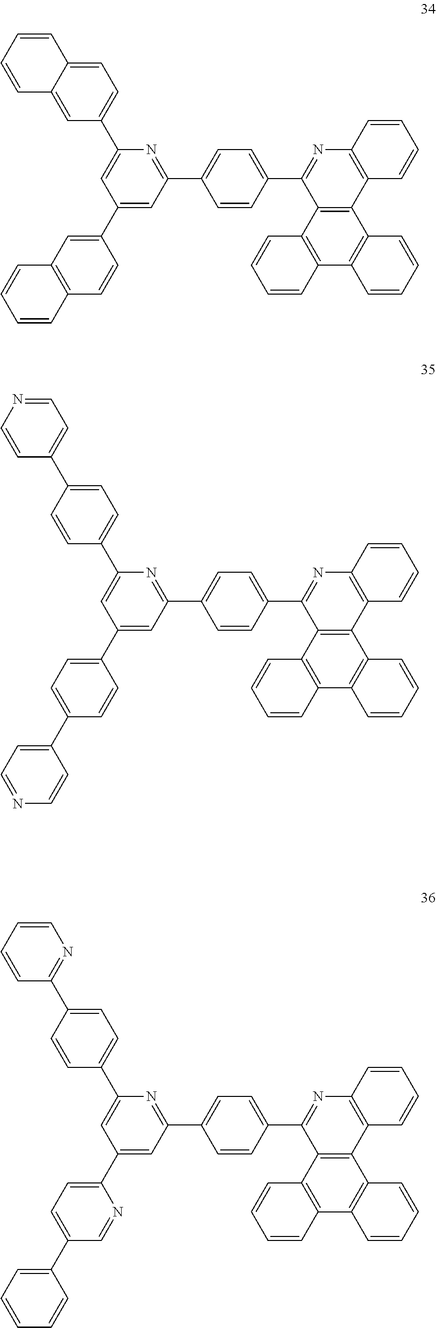 [SMILES: c1ccc2cc(-c3cc(-c4ccc(-c5nc6ccccc6c6c7ccccc7c7ccccc7c56)cc4)nc(-c4ccc5ccccc5c4)c3)ccc2c1 ; c1ccc2c(c1)nc(-c1ccc(-c3cc(-c4ccc(-c5ccncc5)cc4)cc(-c4ccc(-c5ccncc5)cc4)n3)cc1)c1c3ccccc3c3ccccc3c21 ; c1ccc(-c2ccc(-c3cc(-c4ccc(-c5ccccn5)cc4)nc(-c4ccc(-c5nc6ccccc6c6c7ccccc7c7ccccc7c56)cc4)c3)nc2)cc1]

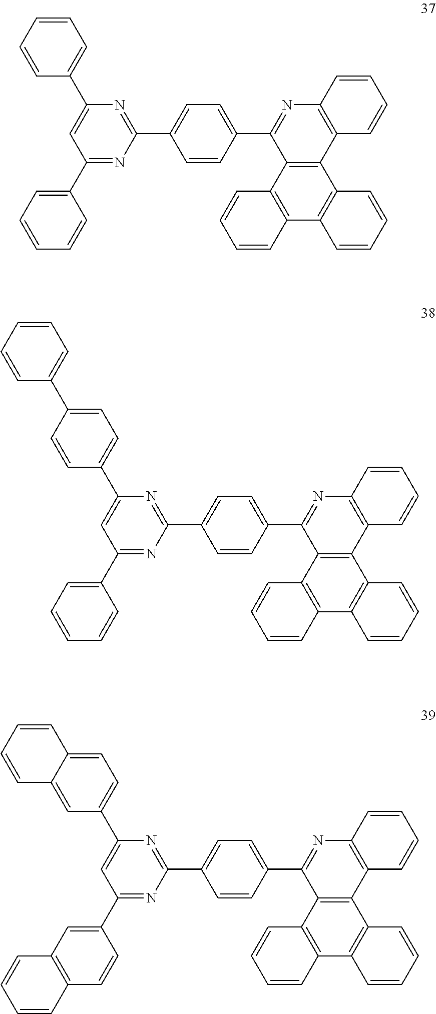 [SMILES: c1ccc(-c2cc(-c3ccccc3)nc(-c3ccc(-c4nc5ccccc5c5c6ccccc6c6ccccc6c45)cc3)n2)cc1 ; c1ccc(-c2ccc(-c3cc(-c4ccccc4)nc(-c4ccc(-c5nc6ccccc6c6c7ccccc7c7ccccc7c56)cc4)n3)cc2)cc1 ; c1ccc2cc(-c3cc(-c4ccc5ccccc5c4)nc(-c4ccc(-c5nc6ccccc6c6c7ccccc7c7ccccc7c56)cc4)n3)ccc2c1]

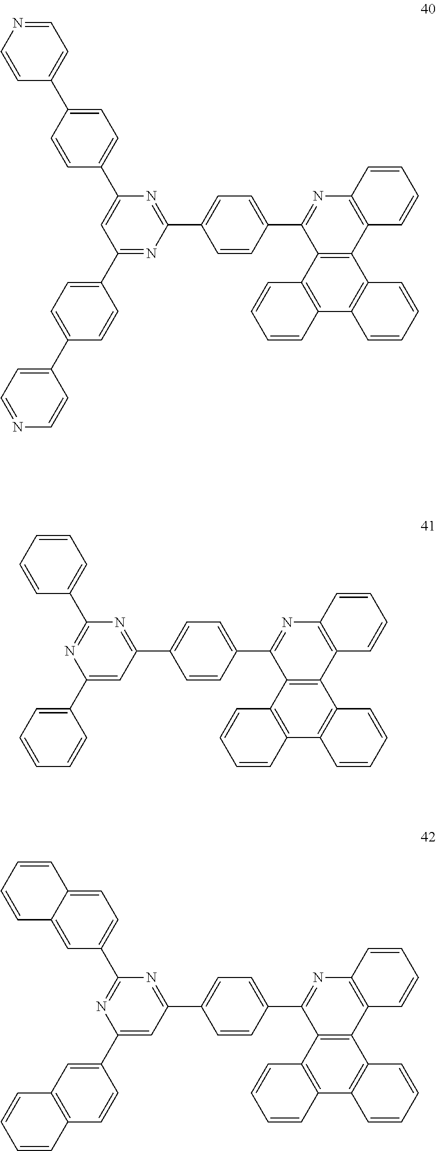 [SMILES: c1ccc2c(c1)nc(-c1ccc(-c3nc(-c4ccc(-c5ccncc5)cc4)cc(-c4ccc(-c5ccncc5)cc4)n3)cc1)c1c3ccccc3c3ccccc3c21 ; c1ccc(-c2cc(-c3ccc(-c4nc5ccccc5c5c6ccccc6c6ccccc6c45)cc3)nc(-c3ccccc3)n2)cc1 ; c1ccc2cc(-c3cc(-c4ccc(-c5nc6ccccc6c6c7ccccc7c7ccccc7c56)cc4)nc(-c4ccc5ccccc5c4)n3)ccc2c1]

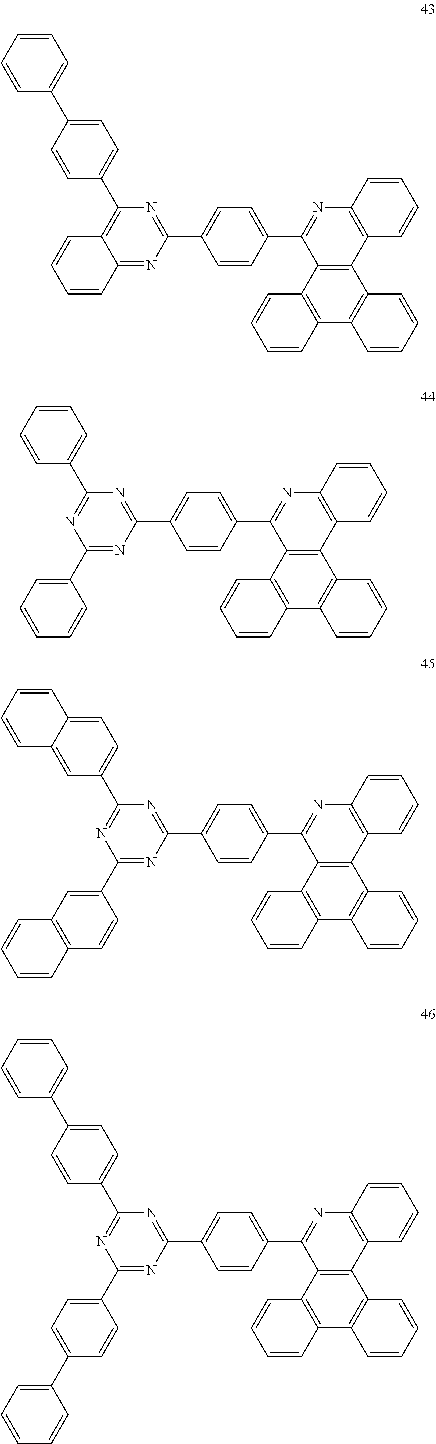 [SMILES: c1ccc(-c2ccc(-c3nc(-c4ccc(-c5nc6ccccc6c6c7ccccc7c7ccccc7c56)cc4)nc4ccccc34)cc2)cc1 ; c1ccc(-c2nc(-c3ccccc3)nc(-c3ccc(-c4nc5ccccc5c5c6ccccc6c6ccccc6c45)cc3)n2)cc1 ; c1ccc2cc(-c3nc(-c4ccc(-c5nc6ccccc6c6c7ccccc7c7ccccc7c56)cc4)nc(-c4ccc5ccccc5c4)n3)ccc2c1 ; c1ccc(-c2ccc(-c3nc(-c4ccc(-c5ccccc5)cc4)nc(-c4ccc(-c5nc6ccccc6c6c7ccccc7c7ccccc7c56)cc4)n3)cc2)cc1]

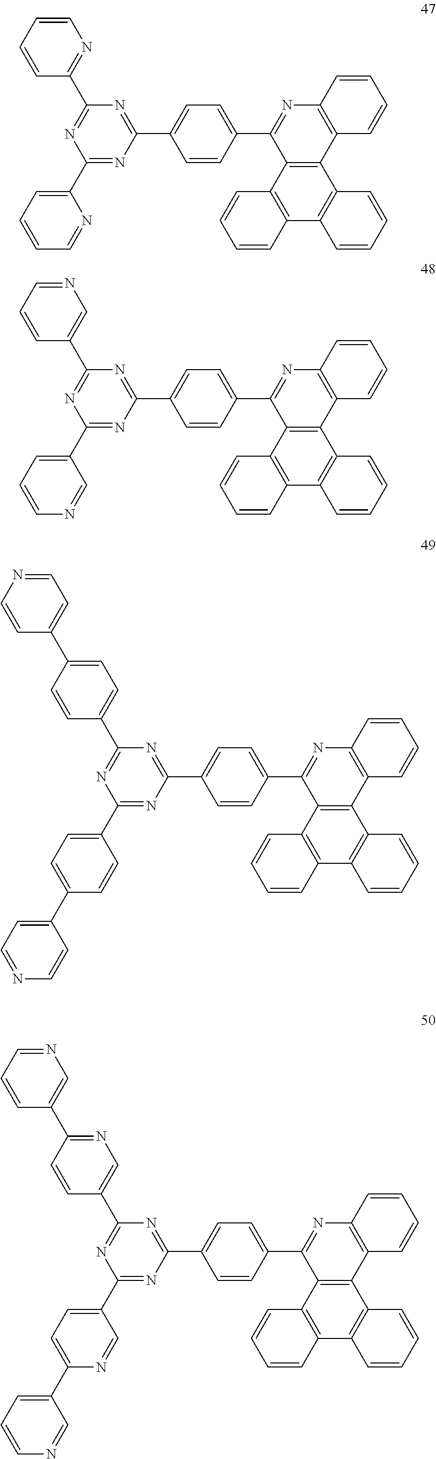

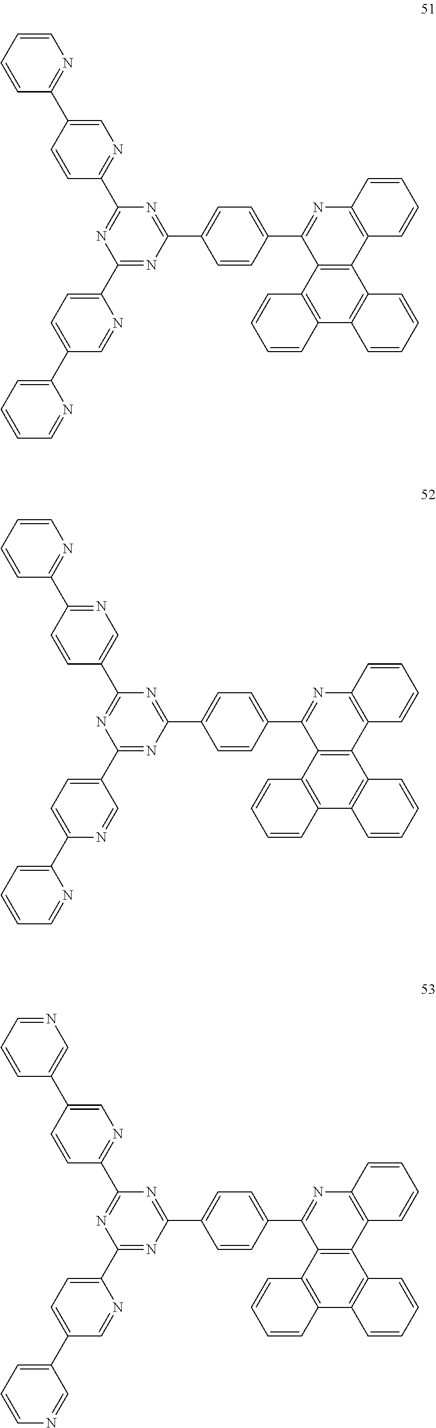 [SMILES: c1ccc(-c2ccc(-c3nc(-c4ccc(-c5nc6ccccc6c6c7ccccc7c7ccccc7c56)cc4)nc(-c4ccc(-c5ccccn5)cn4)n3)nc2)nc1 ; c1ccc(-c2ccc(-c3nc(-c4ccc(-c5nc6ccccc6c6c7ccccc7c7ccccc7c56)cc4)nc(-c4ccc(-c5ccccn5)nc4)n3)cn2)nc1 ; c1cncc(-c2ccc(-c3nc(-c4ccc(-c5nc6ccccc6c6c7ccccc7c7ccccc7c56)cc4)nc(-c4ccc(-c5cccnc5)cn4)n3)nc2)c1]

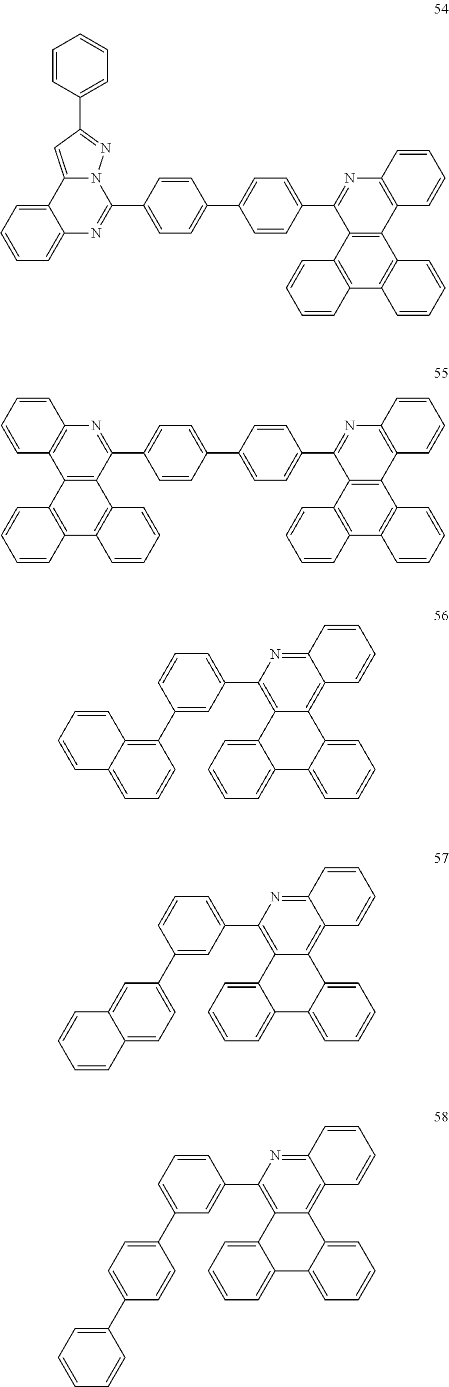 [SMILES: c1ccc(-c2cc3c4ccccc4nc(-c4ccc(-c5ccc(-c6nc7ccccc7c7c8ccccc8c8ccccc8c67)cc5)cc4)n3n2)cc1 ; c1ccc2c(c1)nc(-c1ccc(-c3ccc(-c4nc5ccccc5c5c6ccccc6c6ccccc6c45)cc3)cc1)c1c3ccccc3c3ccccc3c21 ; c1cc(-c2cccc3ccccc23)cc(-c2nc3ccccc3c3c4ccccc4c4ccccc4c23)c1 ; c1cc(-c2ccc3ccccc3c2)cc(-c2nc3ccccc3c3c4ccccc4c4ccccc4c23)c1 ; c1ccc(-c2ccc(-c3cccc(-c4nc5ccccc5c5c6ccccc6c6ccccc6c45)c3)cc2)cc1]

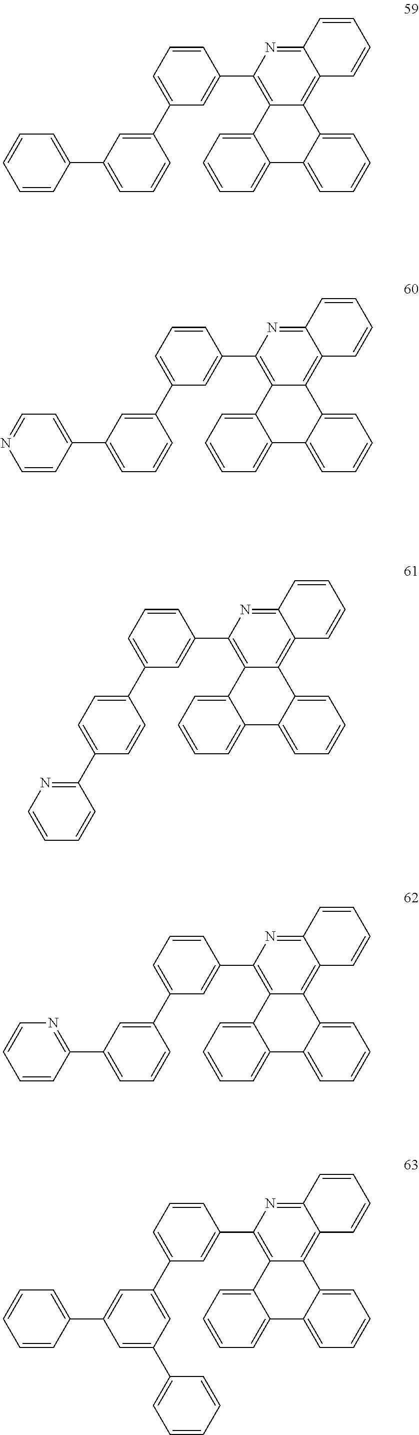 [SMILES: c1ccc(-c2cccc(-c3cccc(-c4nc5ccccc5c5c6ccccc6c6ccccc6c45)c3)c2)cc1 ; c1cc(-c2ccncc2)cc(-c2cccc(-c3nc4ccccc4c4c5ccccc5c5ccccc5c34)c2)c1 ; c1ccc(-c2ccc(-c3cccc(-c4nc5ccccc5c5c6ccccc6c6ccccc6c45)c3)cc2)nc1 ; c1ccc(-c2cccc(-c3cccc(-c4nc5ccccc5c5c6ccccc6c6ccccc6c45)c3)c2)nc1 ; c1ccc(-c2cc(-c3ccccc3)cc(-c3cccc(-c4nc5ccccc5c5c6ccccc6c6ccccc6c45)c3)c2)cc1]

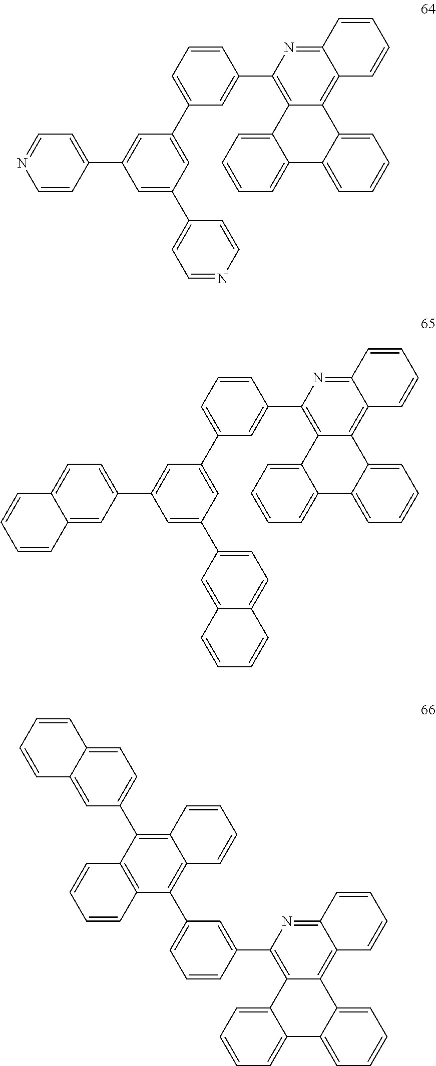 [SMILES: c1cc(-c2cc(-c3ccncc3)cc(-c3ccncc3)c2)cc(-c2nc3ccccc3c3c4ccccc4c4ccccc4c23)c1 ; c1cc(-c2cc(-c3ccc4ccccc4c3)cc(-c3ccc4ccccc4c3)c2)cc(-c2nc3ccccc3c3c4ccccc4c4ccccc4c23)c1 ; c1cc(-c2c3ccccc3c(-c3ccc4ccccc4c3)c3ccccc23)cc(-c2nc3ccccc3c3c4ccccc4c4ccccc4c23)c1]

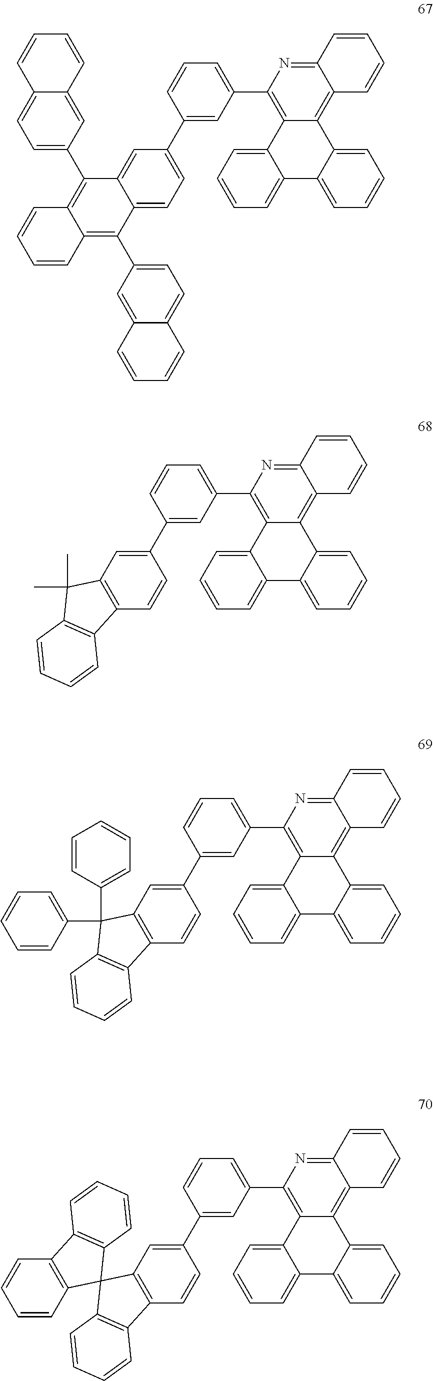 [SMILES: c1cc(-c2ccc3c(-c4ccc5ccccc5c4)c4ccccc4c(-c4ccc5ccccc5c4)c3c2)cc(-c2nc3ccccc3c3c4ccccc4c4ccccc4c23)c1 ; CC1(C)c2ccccc2-c2ccc(-c3cccc(-c4nc5ccccc5c5c6ccccc6c6ccccc6c45)c3)cc21 ; c1ccc(C2(c3ccccc3)c3ccccc3-c3ccc(-c4cccc(-c5nc6ccccc6c6c7ccccc7c7ccccc7c56)c4)cc32)cc1 ; c1cc(-c2ccc3c(c2)C2(c4ccccc4-c4ccccc42)c2ccccc2-3)cc(-c2nc3ccccc3c3c4ccccc4c4ccccc4c23)c1]

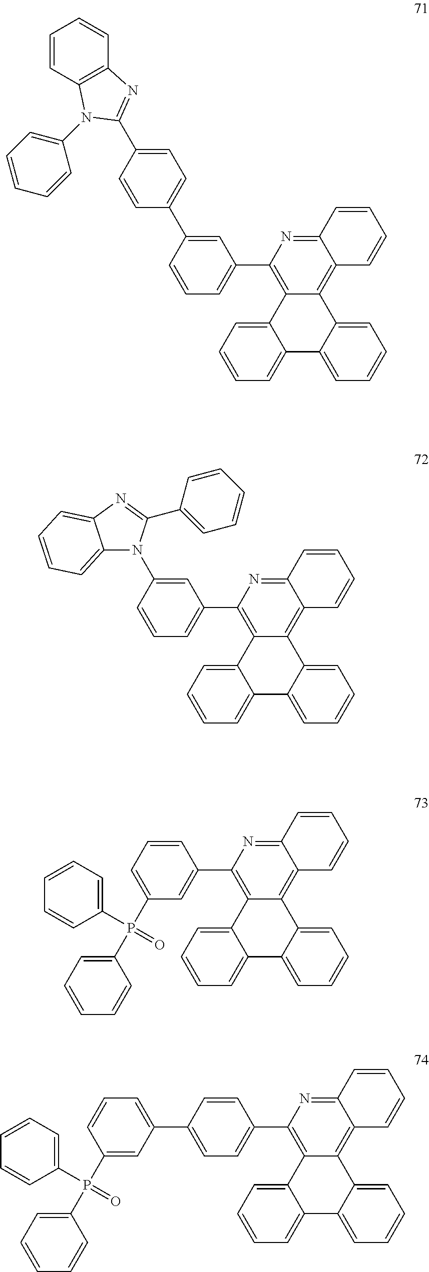 [SMILES: c1ccc(-n2c(-c3ccc(-c4cccc(-c5nc6ccccc6c6c7ccccc7c7ccccc7c56)c4)cc3)nc3ccccc32)cc1 ; c1ccc(-c2nc3ccccc3n2-c2cccc(-c3nc4ccccc4c4c5ccccc5c5ccccc5c34)c2)cc1 ; O=P(c1ccccc1)(c1ccccc1)c1cccc(-c2nc3ccccc3c3c4ccccc4c4ccccc4c23)c1 ; O=P(c1ccccc1)(c1ccccc1)c1cccc(-c2ccc(-c3nc4ccccc4c4c5ccccc5c5ccccc5c34)cc2)c1]

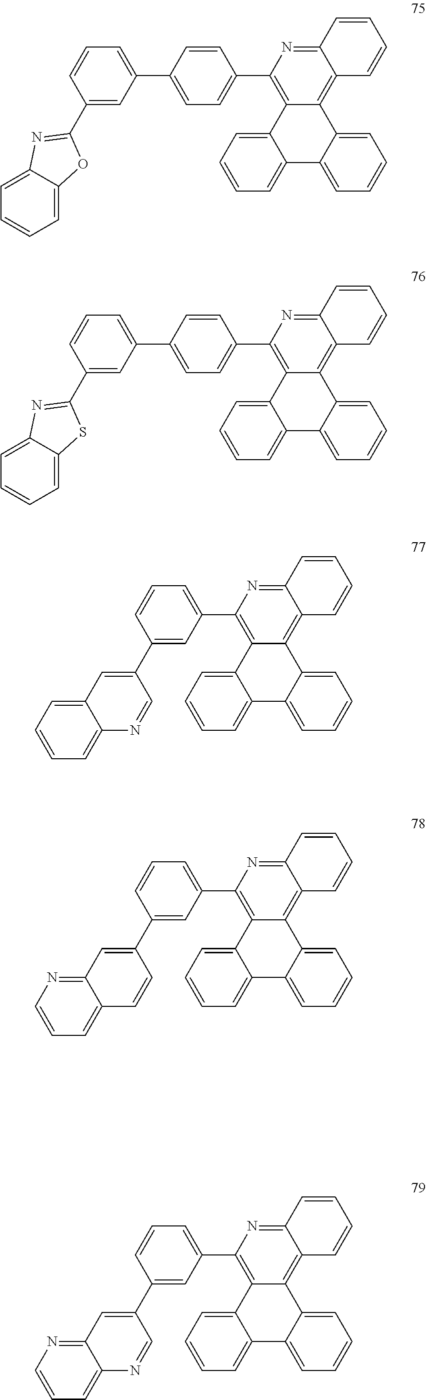 [SMILES: c1cc(-c2ccc(-c3nc4ccccc4c4c5ccccc5c5ccccc5c34)cc2)cc(-c2nc3ccccc3o2)c1 ; c1cc(-c2ccc(-c3nc4ccccc4c4c5ccccc5c5ccccc5c34)cc2)cc(-c2nc3ccccc3s2)c1 ; c1cc(-c2cnc3ccccc3c2)cc(-c2nc3ccccc3c3c4ccccc4c4ccccc4c23)c1 ; c1cc(-c2ccc3cccnc3c2)cc(-c2nc3ccccc3c3c4ccccc4c4ccccc4c23)c1 ; c1cc(-c2cnc3cccnc3c2)cc(-c2nc3ccccc3c3c4ccccc4c4ccccc4c23)c1]

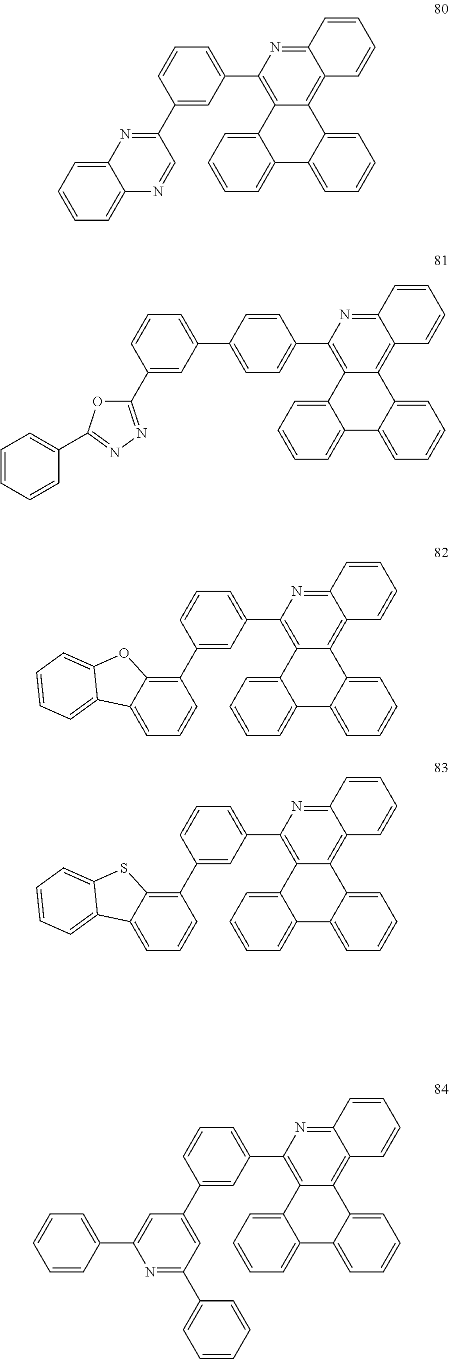

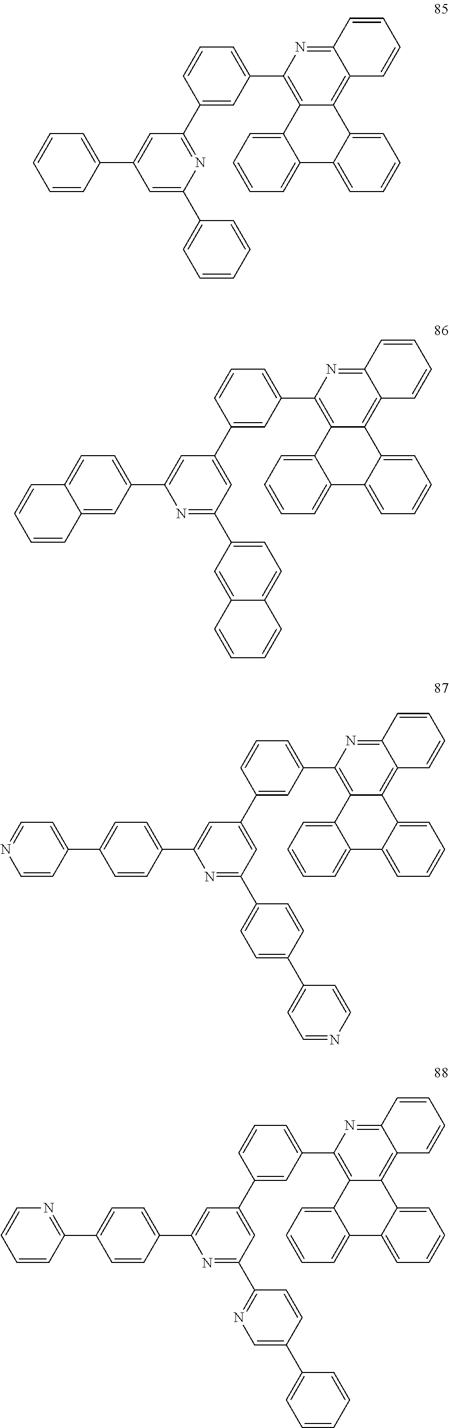 [SMILES: c1ccc(-c2cc(-c3ccccc3)nc(-c3cccc(-c4nc5ccccc5c5c6ccccc6c6ccccc6c45)c3)c2)cc1 ; c1cc(-c2cc(-c3ccc4ccccc4c3)nc(-c3ccc4ccccc4c3)c2)cc(-c2nc3ccccc3c3c4ccccc4c4ccccc4c23)c1 ; c1cc(-c2cc(-c3ccc(-c4ccncc4)cc3)nc(-c3ccc(-c4ccncc4)cc3)c2)cc(-c2nc3ccccc3c3c4ccccc4c4ccccc4c23)c1 ; c1ccc(-c2ccc(-c3cc(-c4cccc(-c5nc6ccccc6c6c7ccccc7c7ccccc7c56)c4)cc(-c4ccc(-c5ccccn5)cc4)n3)nc2)cc1]

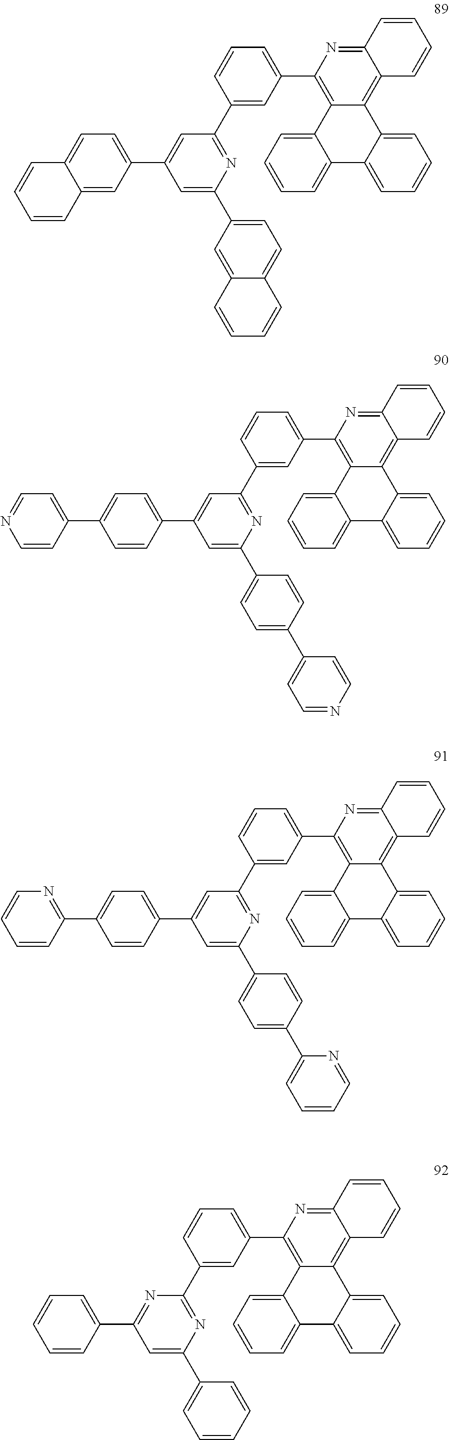 [SMILES: c1cc(-c2cc(-c3ccc4ccccc4c3)cc(-c3ccc4ccccc4c3)n2)cc(-c2nc3ccccc3c3c4ccccc4c4ccccc4c23)c1 ; c1cc(-c2cc(-c3ccc(-c4ccncc4)cc3)cc(-c3ccc(-c4ccncc4)cc3)n2)cc(-c2nc3ccccc3c3c4ccccc4c4ccccc4c23)c1 ; c1ccc(-c2ccc(-c3cc(-c4ccc(-c5ccccn5)cc4)nc(-c4cccc(-c5nc6ccccc6c6c7ccccc7c7ccccc7c56)c4)c3)cc2)nc1 ; c1ccc(-c2cc(-c3ccccc3)nc(-c3cccc(-c4nc5ccccc5c5c6ccccc6c6ccccc6c45)c3)n2)cc1]

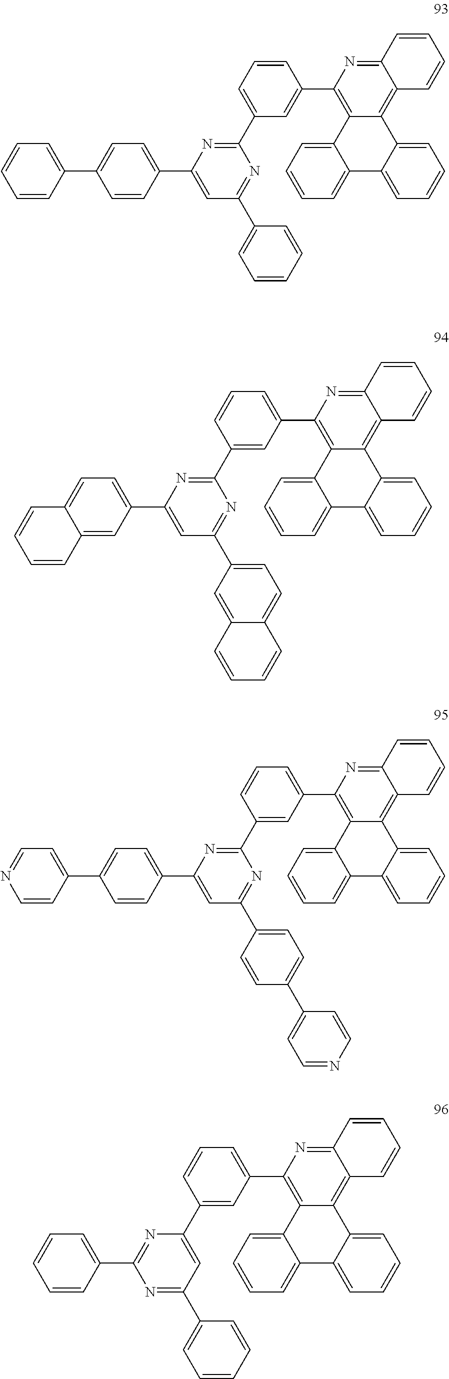 [SMILES: c1ccc(-c2ccc(-c3cc(-c4ccccc4)nc(-c4cccc(-c5nc6ccccc6c6c7ccccc7c7ccccc7c56)c4)n3)cc2)cc1 ; c1cc(-c2nc(-c3ccc4ccccc4c3)cc(-c3ccc4ccccc4c3)n2)cc(-c2nc3ccccc3c3c4ccccc4c4ccccc4c23)c1 ; c1cc(-c2nc(-c3ccc(-c4ccncc4)cc3)cc(-c3ccc(-c4ccncc4)cc3)n2)cc(-c2nc3ccccc3c3c4ccccc4c4ccccc4c23)c1 ; c1ccc(-c2cc(-c3cccc(-c4nc5ccccc5c5c6ccccc6c6ccccc6c45)c3)nc(-c3ccccc3)n2)cc1]

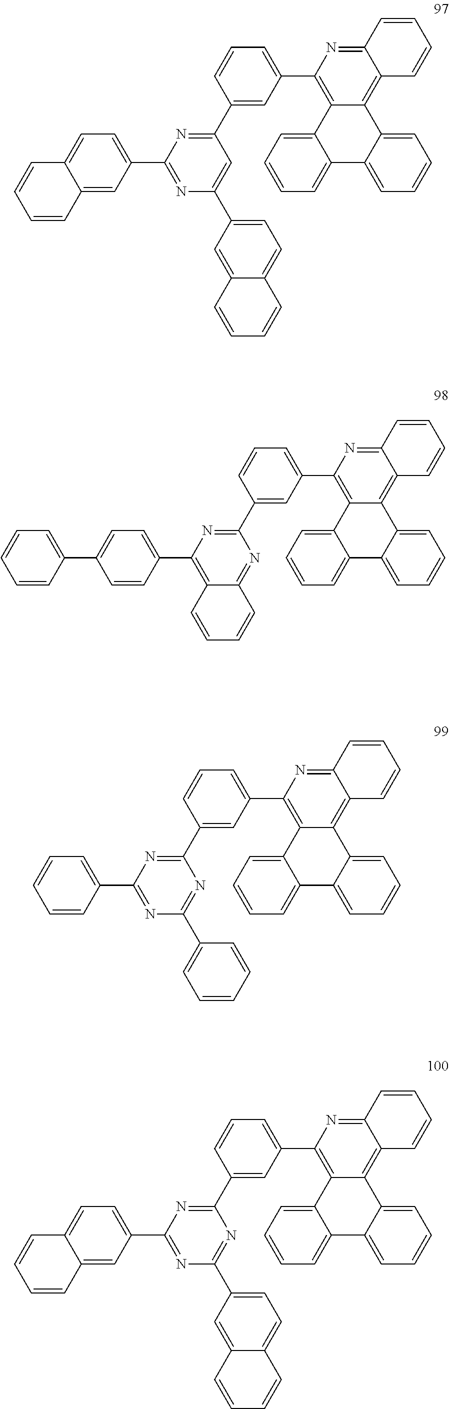 [SMILES: c1cc(-c2cc(-c3ccc4ccccc4c3)nc(-c3ccc4ccccc4c3)n2)cc(-c2nc3ccccc3c3c4ccccc4c4ccccc4c23)c1 ; c1ccc(-c2ccc(-c3nc(-c4cccc(-c5nc6ccccc6c6c7ccccc7c7ccccc7c56)c4)nc4ccccc34)cc2)cc1 ; c1ccc(-c2nc(-c3ccccc3)nc(-c3cccc(-c4nc5ccccc5c5c6ccccc6c6ccccc6c45)c3)n2)cc1 ; c1cc(-c2nc(-c3ccc4ccccc4c3)nc(-c3ccc4ccccc4c3)n2)cc(-c2nc3ccccc3c3c4ccccc4c4ccccc4c23)c1]

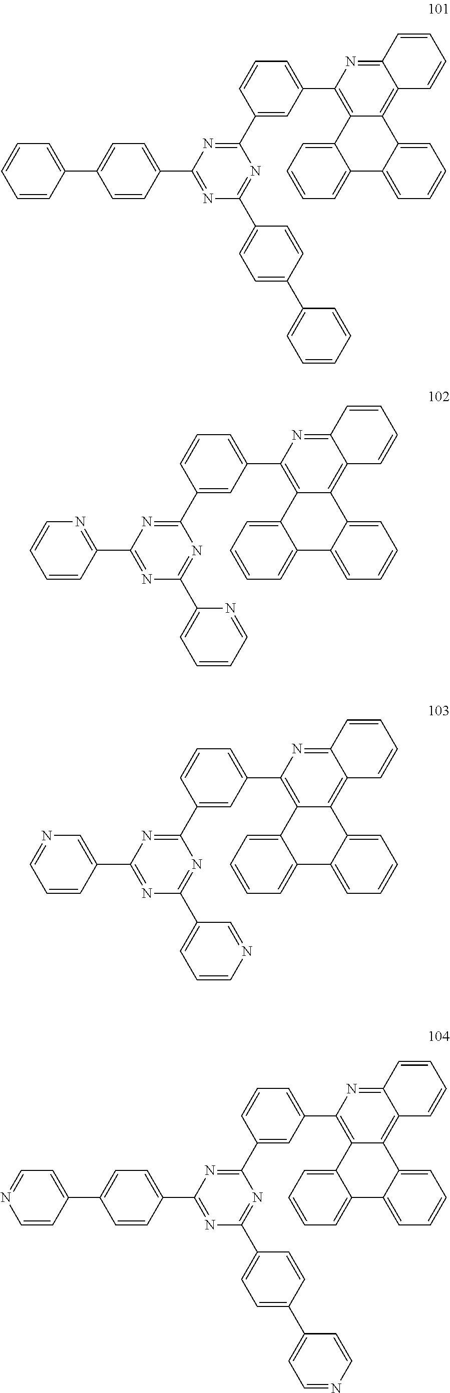 [SMILES: c1ccc(-c2ccc(-c3nc(-c4ccc(-c5ccccc5)cc4)nc(-c4cccc(-c5nc6ccccc6c6c7ccccc7c7ccccc7c56)c4)n3)cc2)cc1 ; c1ccc(-c2nc(-c3cccc(-c4nc5ccccc5c5c6ccccc6c6ccccc6c45)c3)nc(-c3ccccn3)n2)nc1 ; c1cncc(-c2nc(-c3cccnc3)nc(-c3cccc(-c4nc5ccccc5c5c6ccccc6c6ccccc6c45)c3)n2)c1 ; c1cc(-c2nc(-c3ccc(-c4ccncc4)cc3)nc(-c3ccc(-c4ccncc4)cc3)n2)cc(-c2nc3ccccc3c3c4ccccc4c4ccccc4c23)c1]

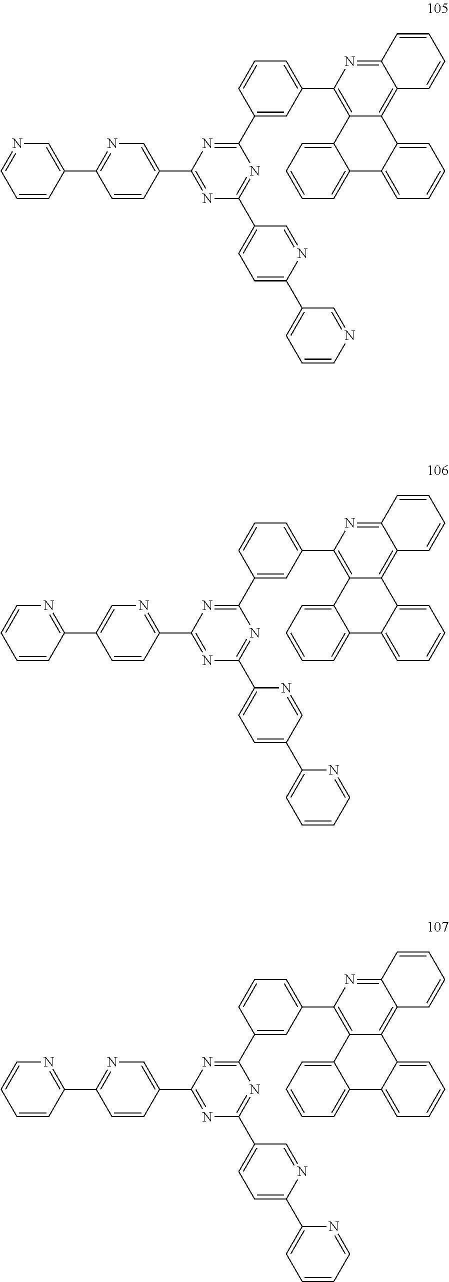 [SMILES: c1cncc(-c2ccc(-c3nc(-c4ccc(-c5cccnc5)nc4)nc(-c4cccc(-c5nc6ccccc6c6c7ccccc7c7ccccc7c56)c4)n3)cn2)c1 ; c1ccc(-c2ccc(-c3nc(-c4cccc(-c5nc6ccccc6c6c7ccccc7c7ccccc7c56)c4)nc(-c4ccc(-c5ccccn5)cn4)n3)nc2)nc1 ; c1ccc(-c2ccc(-c3nc(-c4ccc(-c5ccccn5)nc4)nc(-c4cccc(-c5nc6ccccc6c6c7ccccc7c7ccccc7c56)c4)n3)cn2)nc1]

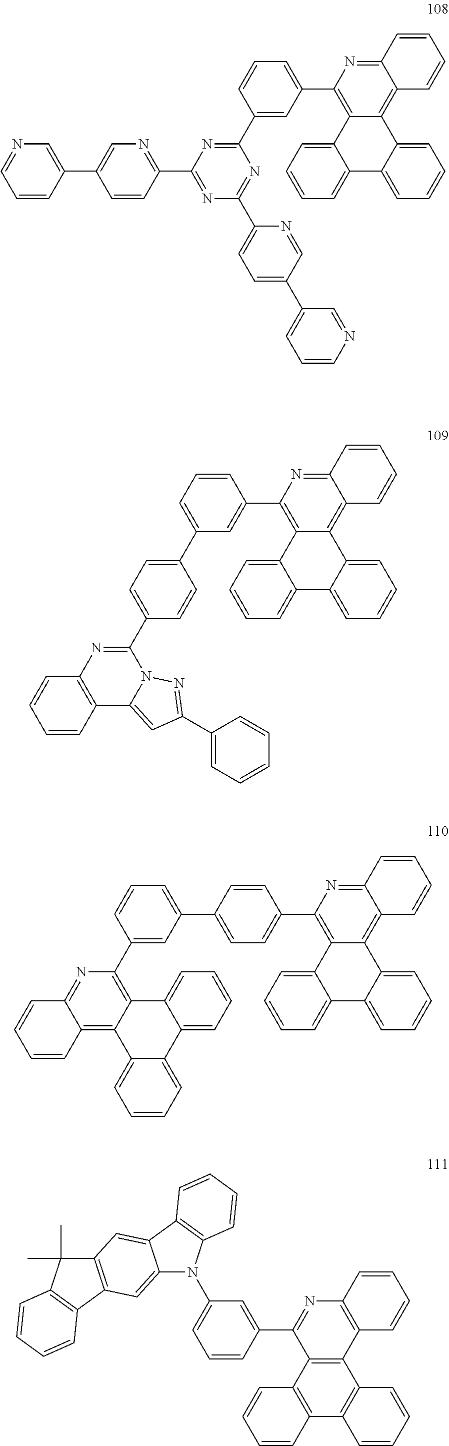 [SMILES: c1cncc(-c2ccc(-c3nc(-c4cccc(-c5nc6ccccc6c6c7ccccc7c7ccccc7c56)c4)nc(-c4ccc(-c5cccnc5)cn4)n3)nc2)c1 ; c1ccc(-c2cc3c4ccccc4nc(-c4ccc(-c5cccc(-c6nc7ccccc7c7c8ccccc8c8ccccc8c67)c5)cc4)n3n2)cc1 ; c1cc(-c2ccc(-c3nc4ccccc4c4c5ccccc5c5ccccc5c34)cc2)cc(-c2nc3ccccc3c3c4ccccc4c4ccccc4c23)c1 ; CC1(C)c2ccccc2-c2cc3c(cc21)c1ccccc1n3-c1cccc(-c2nc3ccccc3c3c4ccccc4c4ccccc4c23)c1]

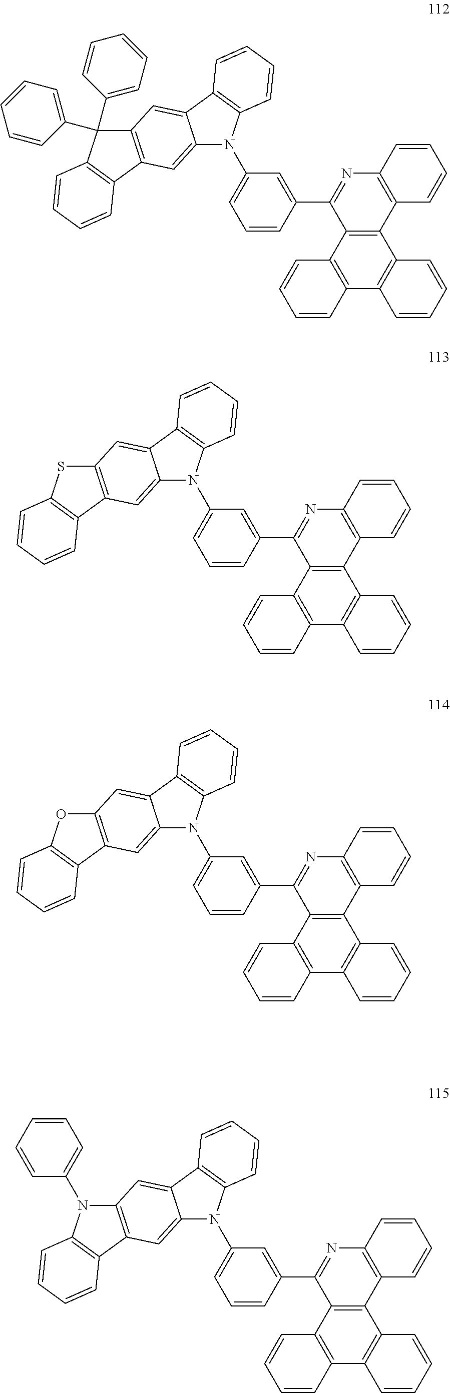 [SMILES: c1ccc(C2(c3ccccc3)c3ccccc3-c3cc4c(cc32)c2ccccc2n4-c2cccc(-c3nc4ccccc4c4c5ccccc5c5ccccc5c34)c2)cc1 ; c1cc(-c2nc3ccccc3c3c4ccccc4c4ccccc4c23)cc(-n2c3ccccc3c3cc4sc5ccccc5c4cc32)c1 ; c1cc(-c2nc3ccccc3c3c4ccccc4c4ccccc4c23)cc(-n2c3ccccc3c3cc4oc5ccccc5c4cc32)c1 ; c1ccc(-n2c3ccccc3c3cc4c(cc32)c2ccccc2n4-c2cccc(-c3nc4ccccc4c4c5ccccc5c5ccccc5c34)c2)cc1]

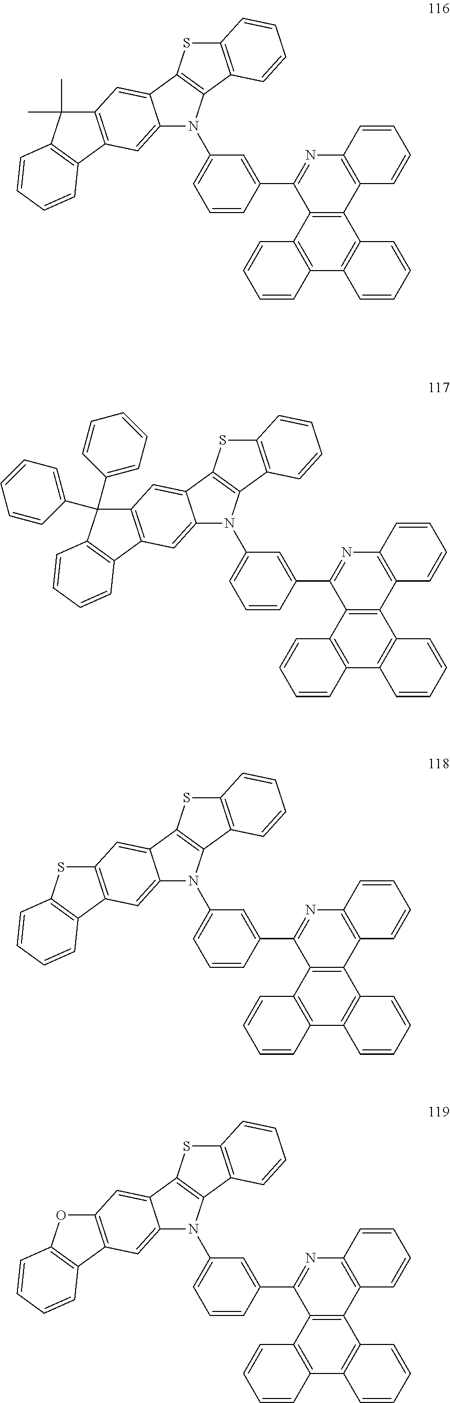 [SMILES: CC1(C)c2ccccc2-c2cc3c(cc21)c1sc2ccccc2c1n3-c1cccc(-c2nc3ccccc3c3c4ccccc4c4ccccc4c23)c1 ; c1ccc(C2(c3ccccc3)c3ccccc3-c3cc4c(cc32)c2sc3ccccc3c2n4-c2cccc(-c3nc4ccccc4c4c5ccccc5c5ccccc5c34)c2)cc1 ; c1cc(-c2nc3ccccc3c3c4ccccc4c4ccccc4c23)cc(-n2c3cc4c(cc3c3sc5ccccc5c32)sc2ccccc24)c1 ; c1cc(-c2nc3ccccc3c3c4ccccc4c4ccccc4c23)cc(-n2c3cc4c(cc3c3sc5ccccc5c32)oc2ccccc24)c1]

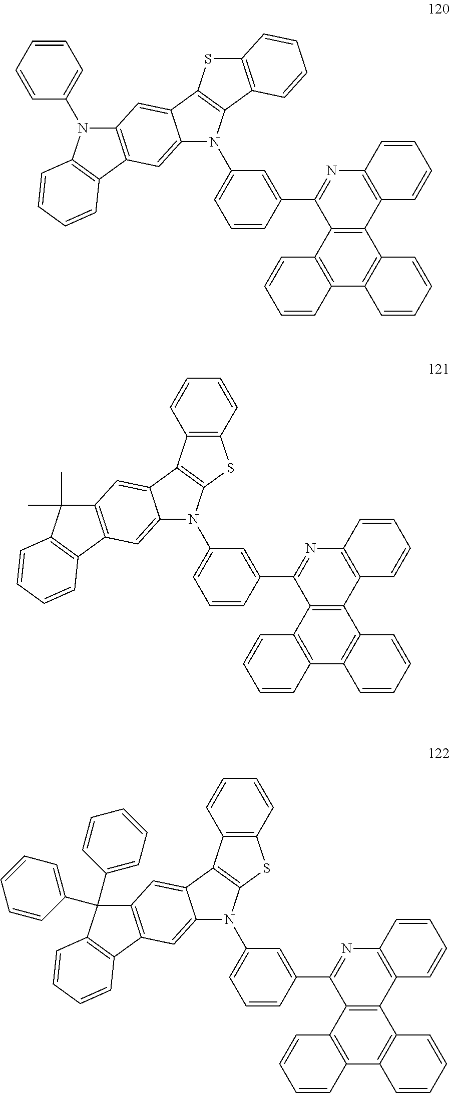 [SMILES: c1ccc(-n2c3ccccc3c3cc4c(cc32)c2sc3ccccc3c2n4-c2cccc(-c3nc4ccccc4c4c5ccccc5c5ccccc5c34)c2)cc1 ; CC1(C)c2ccccc2-c2cc3c(cc21)c1c2ccccc2sc1n3-c1cccc(-c2nc3ccccc3c3c4ccccc4c4ccccc4c23)c1 ; c1ccc(C2(c3ccccc3)c3ccccc3-c3cc4c(cc32)c2c3ccccc3sc2n4-c2cccc(-c3nc4ccccc4c4c5ccccc5c5ccccc5c34)c2)cc1]

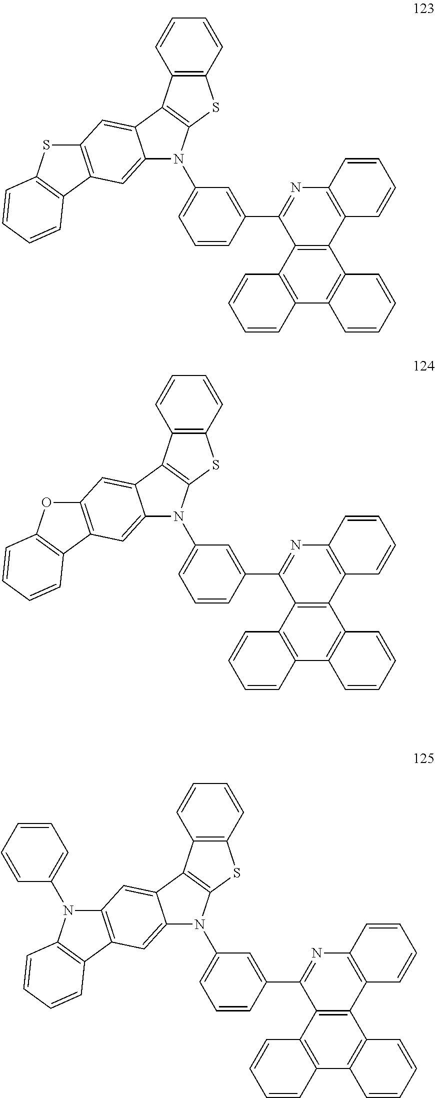 [SMILES: c1cc(-c2nc3ccccc3c3c4ccccc4c4ccccc4c23)cc(-n2c3cc4c(cc3c3c5ccccc5sc32)sc2ccccc24)c1 ; c1cc(-c2nc3ccccc3c3c4ccccc4c4ccccc4c23)cc(-n2c3cc4c(cc3c3c5ccccc5sc32)oc2ccccc24)c1 ; c1ccc(-n2c3ccccc3c3cc4c(cc32)c2c3ccccc3sc2n4-c2cccc(-c3nc4ccccc4c4c5ccccc5c5ccccc5c34)c2)cc1]

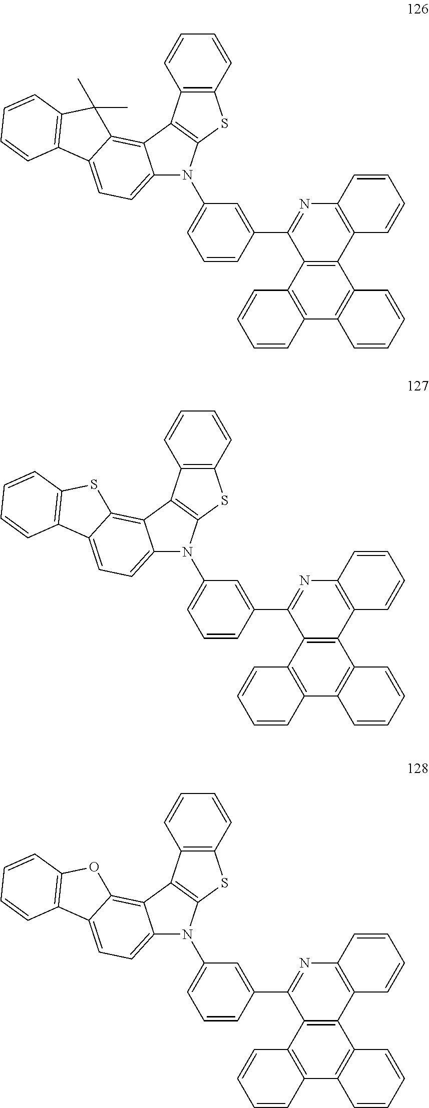 [SMILES: CC1(C)c2ccccc2-c2ccc3c(c21)c1c2ccccc2sc1n3-c1cccc(-c2nc3ccccc3c3c4ccccc4c4ccccc4c23)c1 ; c1cc(-c2nc3ccccc3c3c4ccccc4c4ccccc4c23)cc(-n2c3ccc4c5ccccc5sc4c3c3c4ccccc4sc32)c1 ; c1cc(-c2nc3ccccc3c3c4ccccc4c4ccccc4c23)cc(-n2c3ccc4c5ccccc5oc4c3c3c4ccccc4sc32)c1]

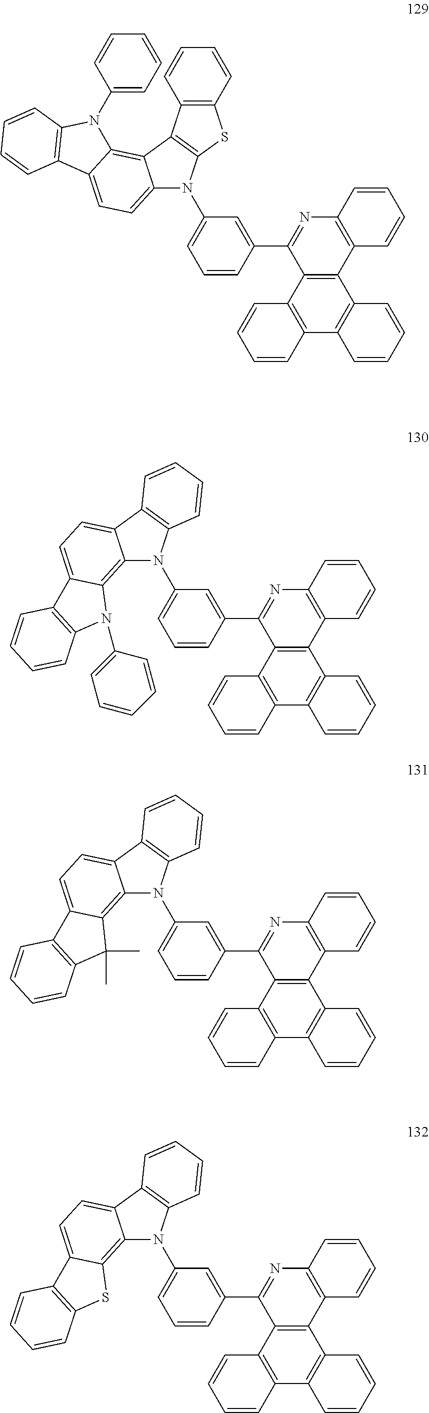 [SMILES: c1ccc(-n2c3ccccc3c3ccc4c(c5c6ccccc6sc5n4-c4cccc(-c5nc6ccccc6c6c7ccccc7c7ccccc7c56)c4)c32)cc1 ; c1ccc(-n2c3ccccc3c3ccc4c5ccccc5n(-c5cccc(-c6nc7ccccc7c7c8ccccc8c8ccccc8c67)c5)c4c32)cc1 ; CC1(C)c2ccccc2-c2ccc3c4ccccc4n(-c4cccc(-c5nc6ccccc6c6c7ccccc7c7ccccc7c56)c4)c3c21 ; c1cc(-c2nc3ccccc3c3c4ccccc4c4ccccc4c23)cc(-n2c3ccccc3c3ccc4c5ccccc5sc4c32)c1]

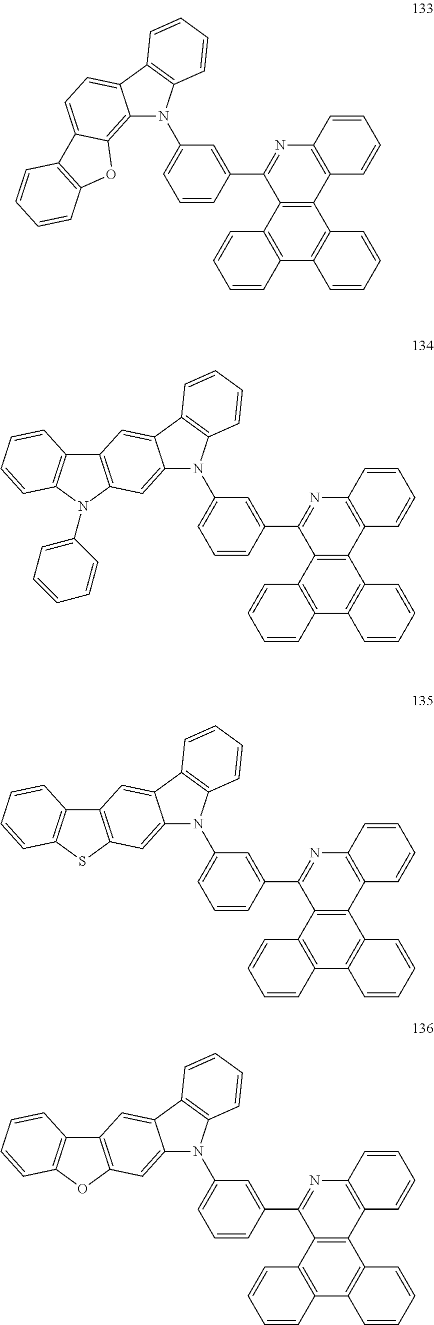 [SMILES: c1cc(-c2nc3ccccc3c3c4ccccc4c4ccccc4c23)cc(-n2c3ccccc3c3ccc4c5ccccc5oc4c32)c1 ; c1ccc(-n2c3ccccc3c3cc4c5ccccc5n(-c5cccc(-c6nc7ccccc7c7c8ccccc8c8ccccc8c67)c5)c4cc32)cc1 ; c1cc(-c2nc3ccccc3c3c4ccccc4c4ccccc4c23)cc(-n2c3ccccc3c3cc4c(cc32)sc2ccccc24)c1 ; c1cc(-c2nc3ccccc3c3c4ccccc4c4ccccc4c23)cc(-n2c3ccccc3c3cc4c(cc32)oc2ccccc24)c1]

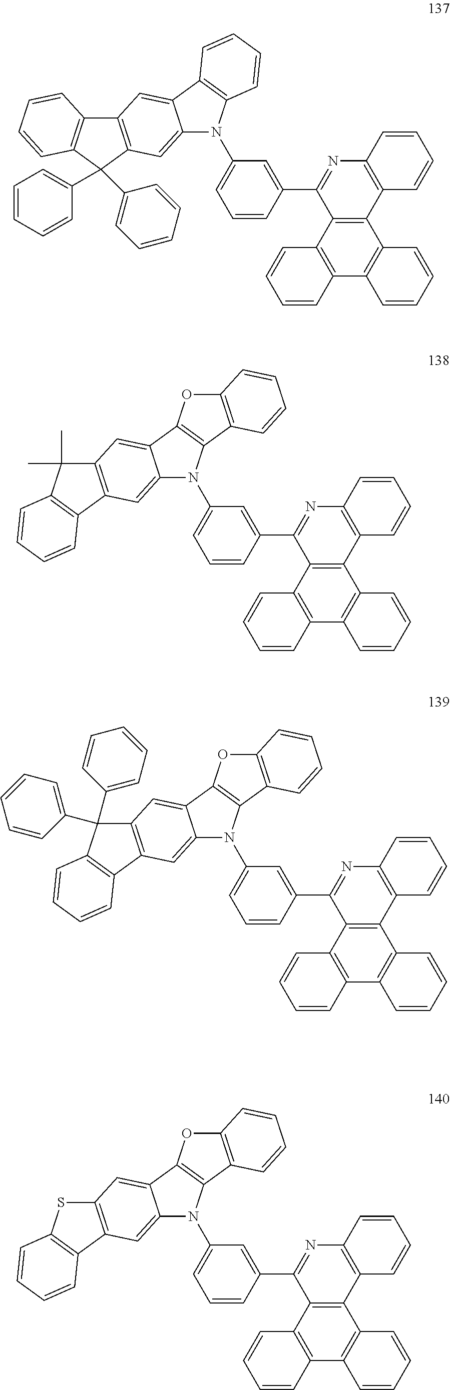 [SMILES: c1ccc(C2(c3ccccc3)c3ccccc3-c3cc4c5ccccc5n(-c5cccc(-c6nc7ccccc7c7c8ccccc8c8ccccc8c67)c5)c4cc32)cc1 ; CC1(C)c2ccccc2-c2cc3c(cc21)c1oc2ccccc2c1n3-c1cccc(-c2nc3ccccc3c3c4ccccc4c4ccccc4c23)c1 ; c1ccc(C2(c3ccccc3)c3ccccc3-c3cc4c(cc32)c2oc3ccccc3c2n4-c2cccc(-c3nc4ccccc4c4c5ccccc5c5ccccc5c34)c2)cc1 ; c1cc(-c2nc3ccccc3c3c4ccccc4c4ccccc4c23)cc(-n2c3cc4c(cc3c3oc5ccccc5c32)sc2ccccc24)c1]

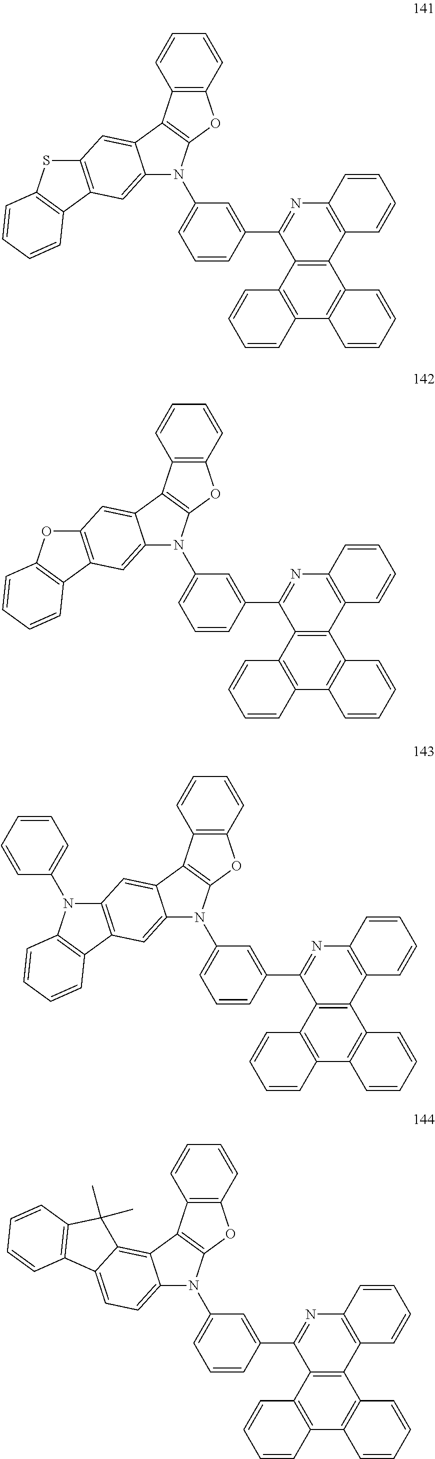 [SMILES: c1cc(-c2nc3ccccc3c3c4ccccc4c4ccccc4c23)cc(-n2c3cc4c(cc3c3c5ccccc5oc32)sc2ccccc24)c1 ; c1cc(-c2nc3ccccc3c3c4ccccc4c4ccccc4c23)cc(-n2c3cc4c(cc3c3c5ccccc5oc32)oc2ccccc24)c1 ; c1ccc(-n2c3ccccc3c3cc4c(cc32)c2c3ccccc3oc2n4-c2cccc(-c3nc4ccccc4c4c5ccccc5c5ccccc5c34)c2)cc1 ; CC1(C)c2ccccc2-c2ccc3c(c21)c1c2ccccc2oc1n3-c1cccc(-c2nc3ccccc3c3c4ccccc4c4ccccc4c23)c1]

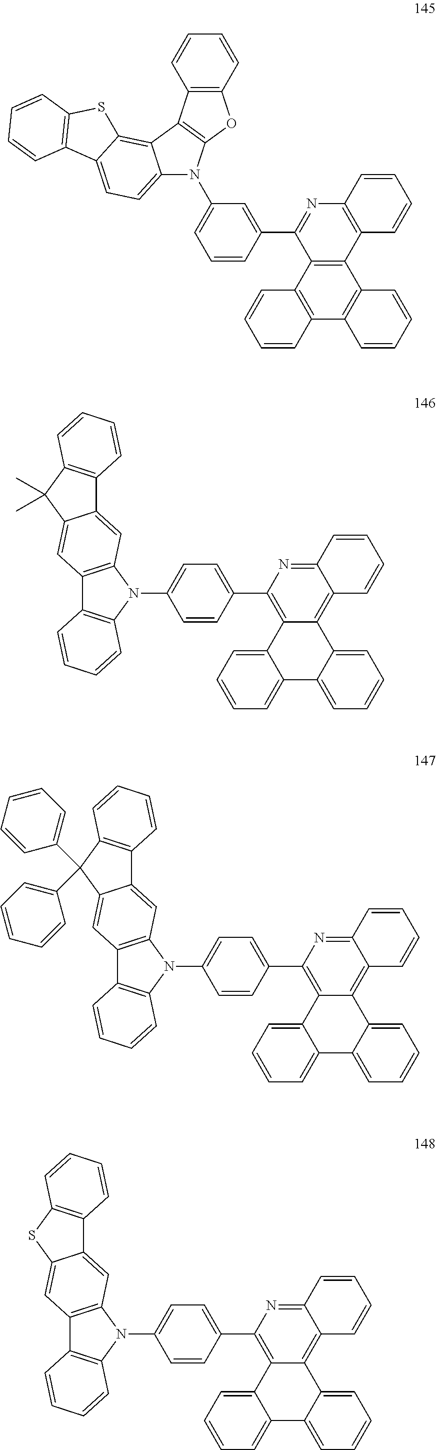 [SMILES: c1cc(-c2nc3ccccc3c3c4ccccc4c4ccccc4c23)cc(-n2c3ccc4c5ccccc5sc4c3c3c4ccccc4oc32)c1 ; CC1(C)c2ccccc2-c2cc3c(cc21)c1ccccc1n3-c1ccc(-c2nc3ccccc3c3c4ccccc4c4ccccc4c23)cc1 ; c1ccc(C2(c3ccccc3)c3ccccc3-c3cc4c(cc32)c2ccccc2n4-c2ccc(-c3nc4ccccc4c4c5ccccc5c5ccccc5c34)cc2)cc1 ; c1ccc2c(c1)nc(-c1ccc(-n3c4ccccc4c4cc5sc6ccccc6c5cc43)cc1)c1c3ccccc3c3ccccc3c21]

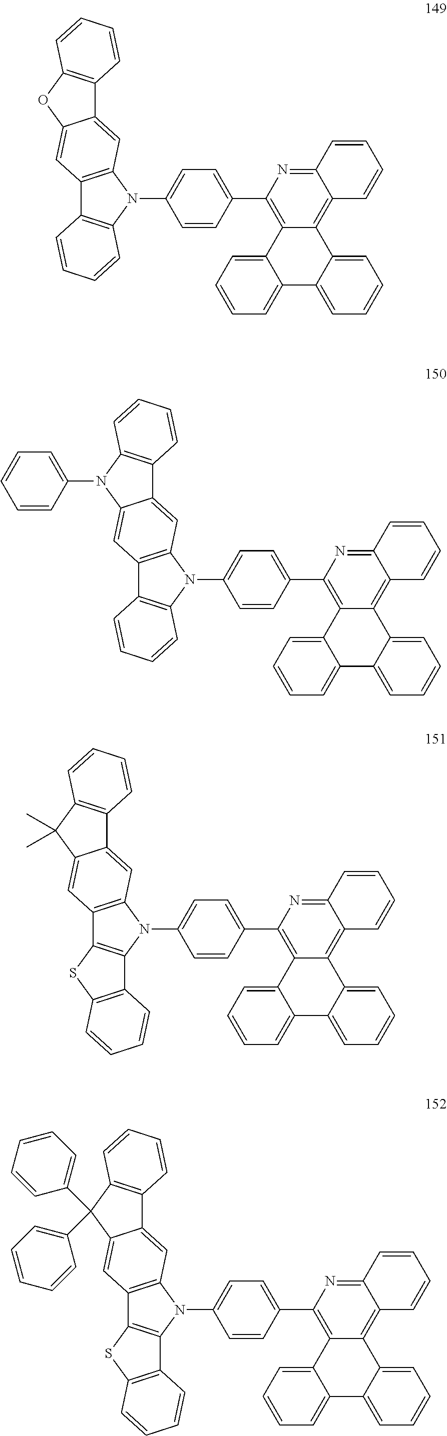 [SMILES: c1ccc2c(c1)nc(-c1ccc(-n3c4ccccc4c4cc5oc6ccccc6c5cc43)cc1)c1c3ccccc3c3ccccc3c21 ; c1ccc(-n2c3ccccc3c3cc4c(cc32)c2ccccc2n4-c2ccc(-c3nc4ccccc4c4c5ccccc5c5ccccc5c34)cc2)cc1 ; CC1(C)c2ccccc2-c2cc3c(cc21)c1sc2ccccc2c1n3-c1ccc(-c2nc3ccccc3c3c4ccccc4c4ccccc4c23)cc1 ; c1ccc(C2(c3ccccc3)c3ccccc3-c3cc4c(cc32)c2sc3ccccc3c2n4-c2ccc(-c3nc4ccccc4c4c5ccccc5c5ccccc5c34)cc2)cc1]

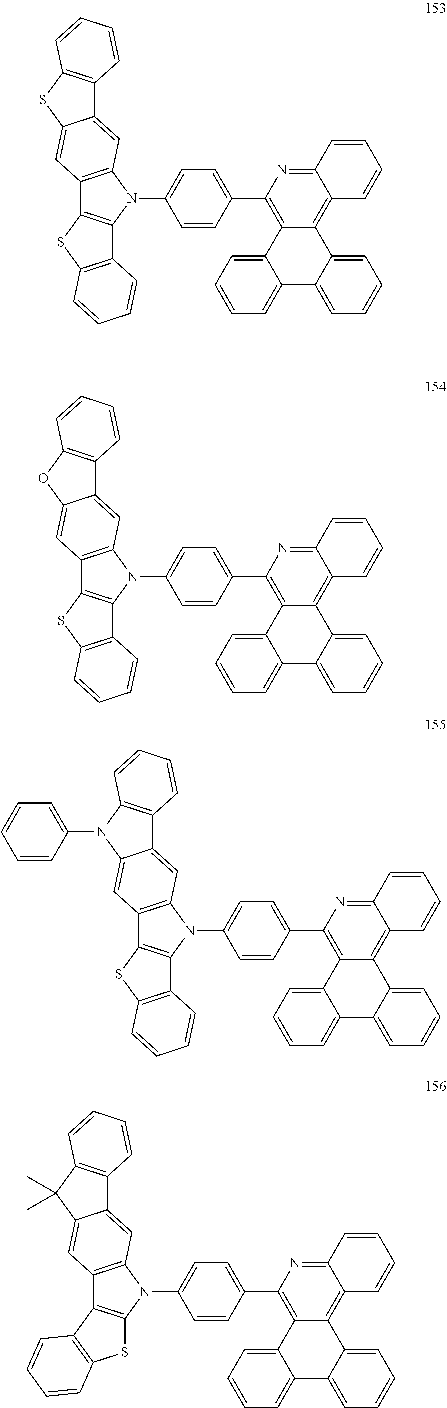 [SMILES: c1ccc2c(c1)nc(-c1ccc(-n3c4cc5c(cc4c4sc6ccccc6c43)sc3ccccc35)cc1)c1c3ccccc3c3ccccc3c21 ; c1ccc2c(c1)nc(-c1ccc(-n3c4cc5c(cc4c4sc6ccccc6c43)oc3ccccc35)cc1)c1c3ccccc3c3ccccc3c21 ; c1ccc(-n2c3ccccc3c3cc4c(cc32)c2sc3ccccc3c2n4-c2ccc(-c3nc4ccccc4c4c5ccccc5c5ccccc5c34)cc2)cc1 ; CC1(C)c2ccccc2-c2cc3c(cc21)c1c2ccccc2sc1n3-c1ccc(-c2nc3ccccc3c3c4ccccc4c4ccccc4c23)cc1]

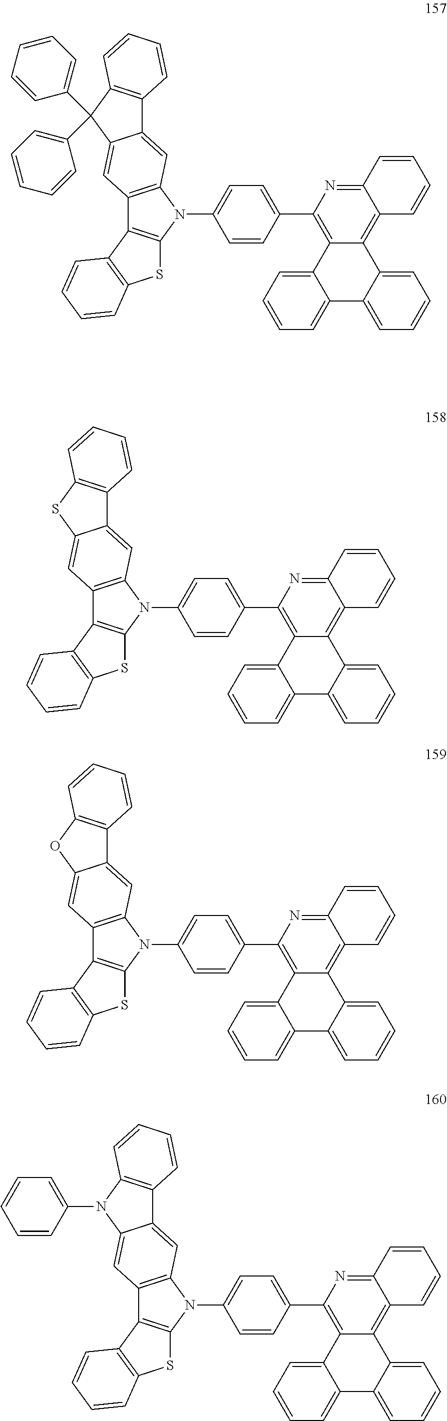 [SMILES: c1ccc(C2(c3ccccc3)c3ccccc3-c3cc4c(cc32)c2c3ccccc3sc2n4-c2ccc(-c3nc4ccccc4c4c5ccccc5c5ccccc5c34)cc2)cc1 ; c1ccc2c(c1)nc(-c1ccc(-n3c4cc5c(cc4c4c6ccccc6sc43)sc3ccccc35)cc1)c1c3ccccc3c3ccccc3c21 ; c1ccc2c(c1)nc(-c1ccc(-n3c4cc5c(cc4c4c6ccccc6sc43)oc3ccccc35)cc1)c1c3ccccc3c3ccccc3c21 ; c1ccc(-n2c3ccccc3c3cc4c(cc32)c2c3ccccc3sc2n4-c2ccc(-c3nc4ccccc4c4c5ccccc5c5ccccc5c34)cc2)cc1]

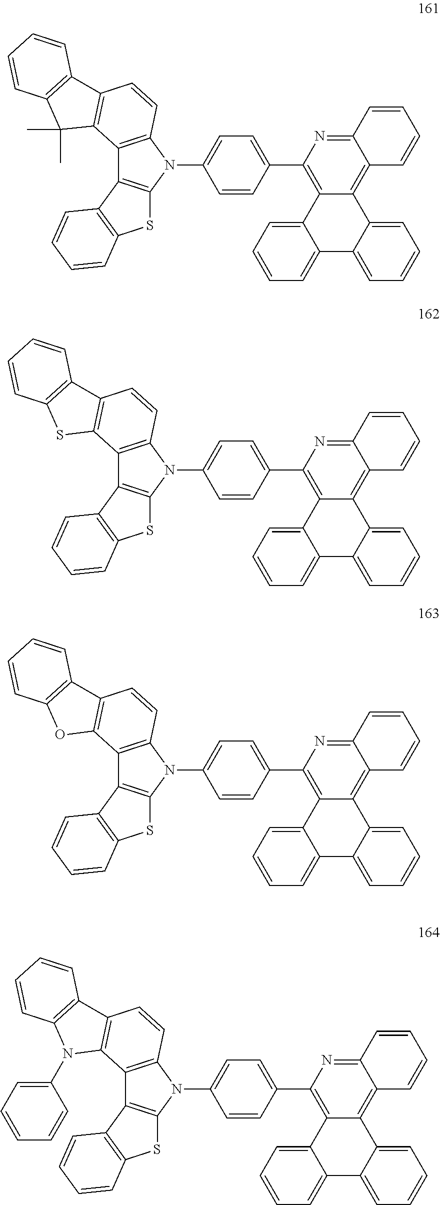 [SMILES: CC1(C)c2ccccc2-c2ccc3c(c21)c1c2ccccc2sc1n3-c1ccc(-c2nc3ccccc3c3c4ccccc4c4ccccc4c23)cc1 ; c1ccc2c(c1)nc(-c1ccc(-n3c4ccc5c6ccccc6sc5c4c4c5ccccc5sc43)cc1)c1c3ccccc3c3ccccc3c21 ; c1ccc2c(c1)nc(-c1ccc(-n3c4ccc5c6ccccc6oc5c4c4c5ccccc5sc43)cc1)c1c3ccccc3c3ccccc3c21 ; c1ccc(-n2c3ccccc3c3ccc4c(c5c6ccccc6sc5n4-c4ccc(-c5nc6ccccc6c6c7ccccc7c7ccccc7c56)cc4)c32)cc1]

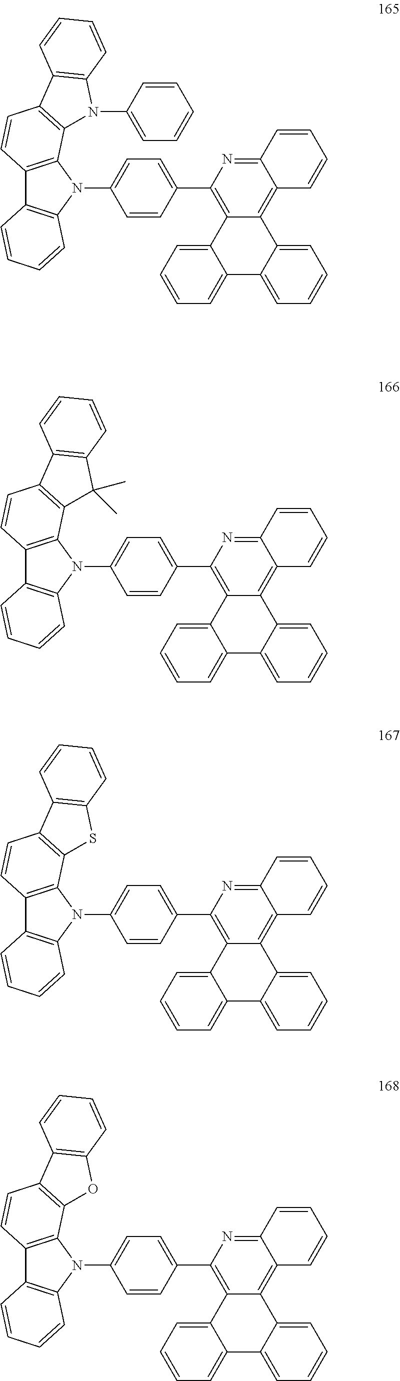 [SMILES: c1ccc(-n2c3ccccc3c3ccc4c5ccccc5n(-c5ccc(-c6nc7ccccc7c7c8ccccc8c8ccccc8c67)cc5)c4c32)cc1 ; CC1(C)c2ccccc2-c2ccc3c4ccccc4n(-c4ccc(-c5nc6ccccc6c6c7ccccc7c7ccccc7c56)cc4)c3c21 ; c1ccc2c(c1)nc(-c1ccc(-n3c4ccccc4c4ccc5c6ccccc6sc5c43)cc1)c1c3ccccc3c3ccccc3c21 ; c1ccc2c(c1)nc(-c1ccc(-n3c4ccccc4c4ccc5c6ccccc6oc5c43)cc1)c1c3ccccc3c3ccccc3c21]

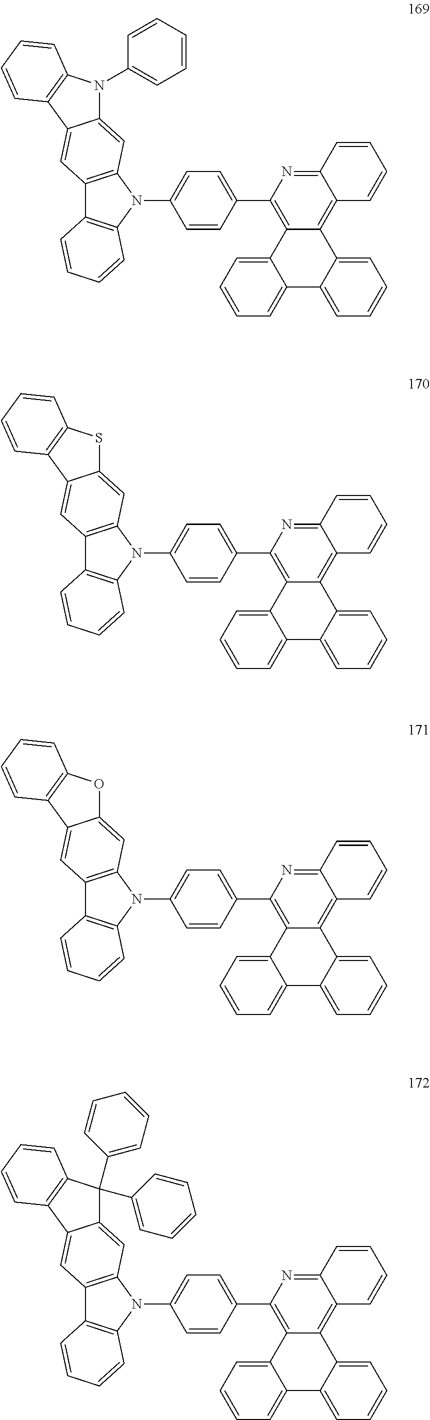 [SMILES: c1ccc(-n2c3ccccc3c3cc4c5ccccc5n(-c5ccc(-c6nc7ccccc7c7c8ccccc8c8ccccc8c67)cc5)c4cc32)cc1 ; c1ccc2c(c1)nc(-c1ccc(-n3c4ccccc4c4cc5c(cc43)sc3ccccc35)cc1)c1c3ccccc3c3ccccc3c21 ; c1ccc2c(c1)nc(-c1ccc(-n3c4ccccc4c4cc5c(cc43)oc3ccccc35)cc1)c1c3ccccc3c3ccccc3c21 ; c1ccc(C2(c3ccccc3)c3ccccc3-c3cc4c5ccccc5n(-c5ccc(-c6nc7ccccc7c7c8ccccc8c8ccccc8c67)cc5)c4cc32)cc1]

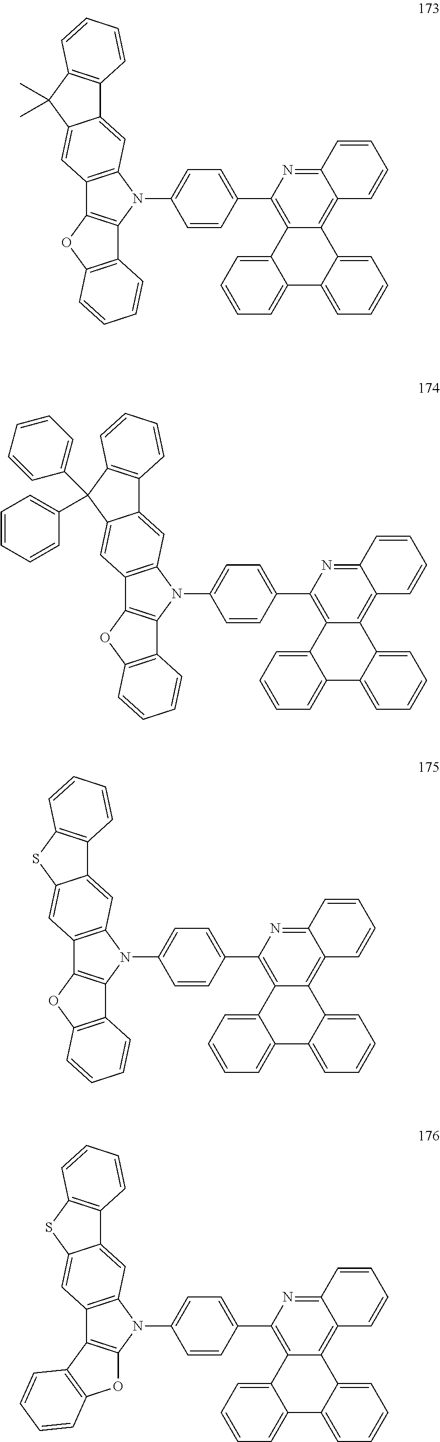 [SMILES: CC1(C)c2ccccc2-c2cc3c(cc21)c1oc2ccccc2c1n3-c1ccc(-c2nc3ccccc3c3c4ccccc4c4ccccc4c23)cc1 ; c1ccc(C2(c3ccccc3)c3ccccc3-c3cc4c(cc32)c2oc3ccccc3c2n4-c2ccc(-c3nc4ccccc4c4c5ccccc5c5ccccc5c34)cc2)cc1 ; c1ccc2c(c1)nc(-c1ccc(-n3c4cc5c(cc4c4oc6ccccc6c43)sc3ccccc35)cc1)c1c3ccccc3c3ccccc3c21 ; c1ccc2c(c1)nc(-c1ccc(-n3c4cc5c(cc4c4c6ccccc6oc43)sc3ccccc35)cc1)c1c3ccccc3c3ccccc3c21]

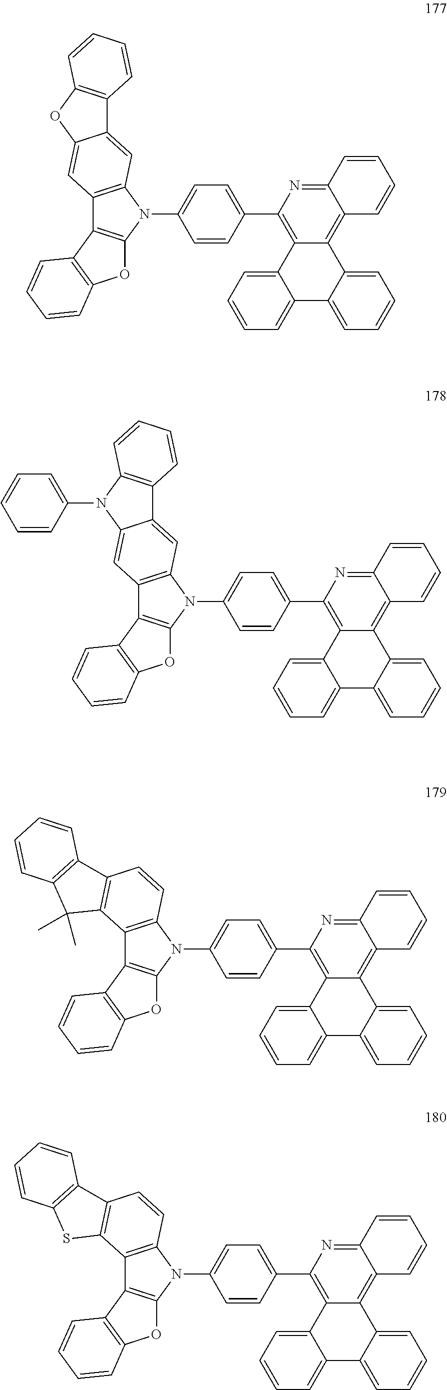 [SMILES: c1ccc2c(c1)nc(-c1ccc(-n3c4cc5c(cc4c4c6ccccc6oc43)oc3ccccc35)cc1)c1c3ccccc3c3ccccc3c21 ; c1ccc(-n2c3ccccc3c3cc4c(cc32)c2c3ccccc3oc2n4-c2ccc(-c3nc4ccccc4c4c5ccccc5c5ccccc5c34)cc2)cc1 ; CC1(C)c2ccccc2-c2ccc3c(c21)c1c2ccccc2oc1n3-c1ccc(-c2nc3ccccc3c3c4ccccc4c4ccccc4c23)cc1 ; c1ccc2c(c1)nc(-c1ccc(-n3c4ccc5c6ccccc6sc5c4c4c5ccccc5oc43)cc1)c1c3ccccc3c3ccccc3c21]

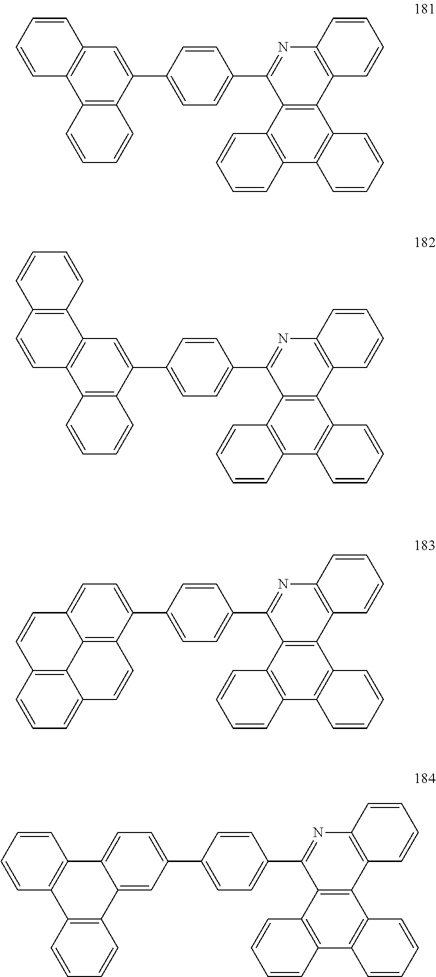 [SMILES: c1ccc2c(c1)cc(-c1ccc(-c3nc4ccccc4c4c5ccccc5c5ccccc5c34)cc1)c1ccccc12 ; c1ccc2c(c1)ccc1c3ccccc3c(-c3ccc(-c4nc5ccccc5c5c6ccccc6c6ccccc6c45)cc3)cc21 ; c1cc2ccc3ccc(-c4ccc(-c5nc6ccccc6c6c7ccccc7c7ccccc7c56)cc4)c4ccc(c1)c2c34 ; c1ccc2c(c1)nc(-c1ccc(-c3ccc4c5ccccc5c5ccccc5c4c3)cc1)c1c3ccccc3c3ccccc3c21]

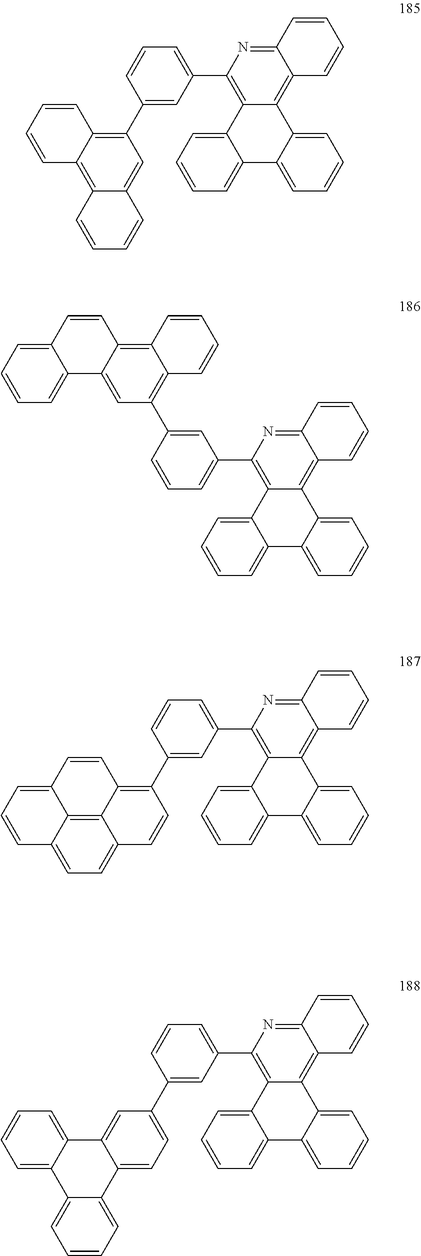 [SMILES: c1cc(-c2cc3ccccc3c3ccccc23)cc(-c2nc3ccccc3c3c4ccccc4c4ccccc4c23)c1 ; c1cc(-c2cc3c4ccccc4ccc3c3ccccc23)cc(-c2nc3ccccc3c3c4ccccc4c4ccccc4c23)c1 ; c1cc(-c2ccc3ccc4cccc5ccc2c3c45)cc(-c2nc3ccccc3c3c4ccccc4c4ccccc4c23)c1 ; c1cc(-c2ccc3c4ccccc4c4ccccc4c3c2)cc(-c2nc3ccccc3c3c4ccccc4c4ccccc4c23)c1]

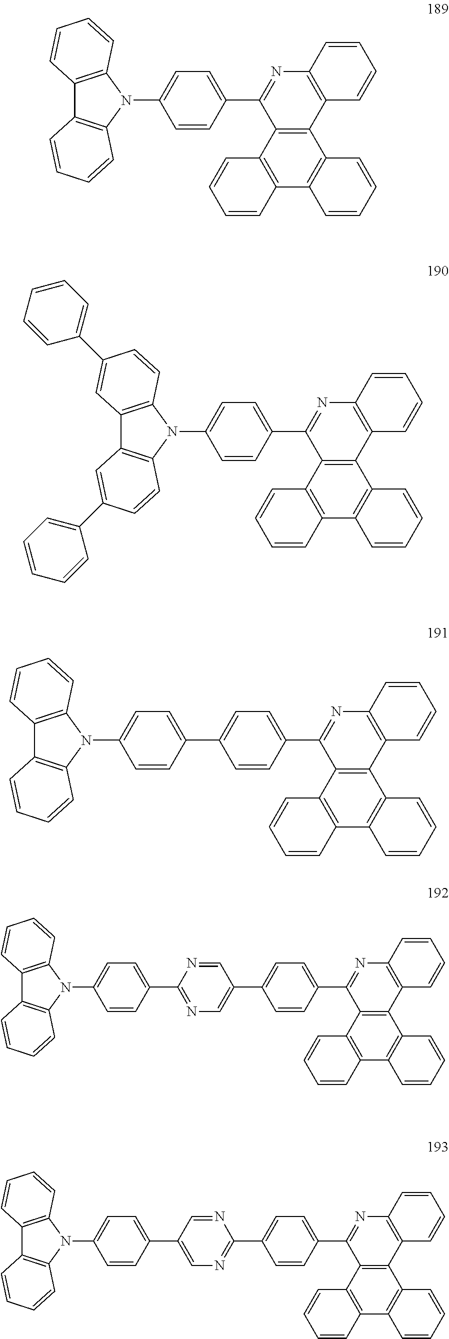 [SMILES: c1ccc2c(c1)nc(-c1ccc(-n3c4ccccc4c4ccccc43)cc1)c1c3ccccc3c3ccccc3c21 ; c1ccc(-c2ccc3c(c2)c2cc(-c4ccccc4)ccc2n3-c2ccc(-c3nc4ccccc4c4c5ccccc5c5ccccc5c34)cc2)cc1 ; c1ccc2c(c1)nc(-c1ccc(-c3ccc(-n4c5ccccc5c5ccccc54)cc3)cc1)c1c3ccccc3c3ccccc3c21 ; c1ccc2c(c1)nc(-c1ccc(-c3cnc(-c4ccc(-n5c6ccccc6c6ccccc65)cc4)nc3)cc1)c1c3ccccc3c3ccccc3c21 ; c1ccc2c(c1)nc(-c1ccc(-c3ncc(-c4ccc(-n5c6ccccc6c6ccccc65)cc4)cn3)cc1)c1c3ccccc3c3ccccc3c21]

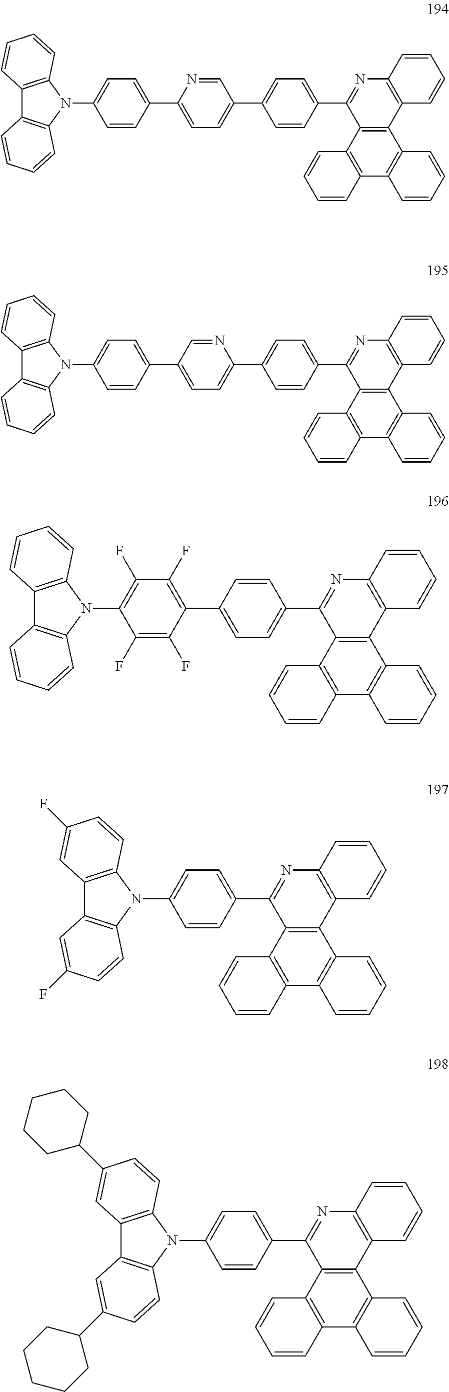 [SMILES: c1ccc2c(c1)nc(-c1ccc(-c3ccc(-c4ccc(-n5c6ccccc6c6ccccc65)cc4)nc3)cc1)c1c3ccccc3c3ccccc3c21 ; c1ccc2c(c1)nc(-c1ccc(-c3ccc(-c4ccc(-n5c6ccccc6c6ccccc65)cc4)cn3)cc1)c1c3ccccc3c3ccccc3c21 ; Fc1c(F)c(-n2c3ccccc3c3ccccc32)c(F)c(F)c1-c1ccc(-c2nc3ccccc3c3c4ccccc4c4ccccc4c23)cc1 ; Fc1ccc2c(c1)c1cc(F)ccc1n2-c1ccc(-c2nc3ccccc3c3c4ccccc4c4ccccc4c23)cc1 ; c1ccc2c(c1)nc(-c1ccc(-n3c4ccc(C5CCCCC5)cc4c4cc(C5CCCCC5)ccc43)cc1)c1c3ccccc3c3ccccc3c21]

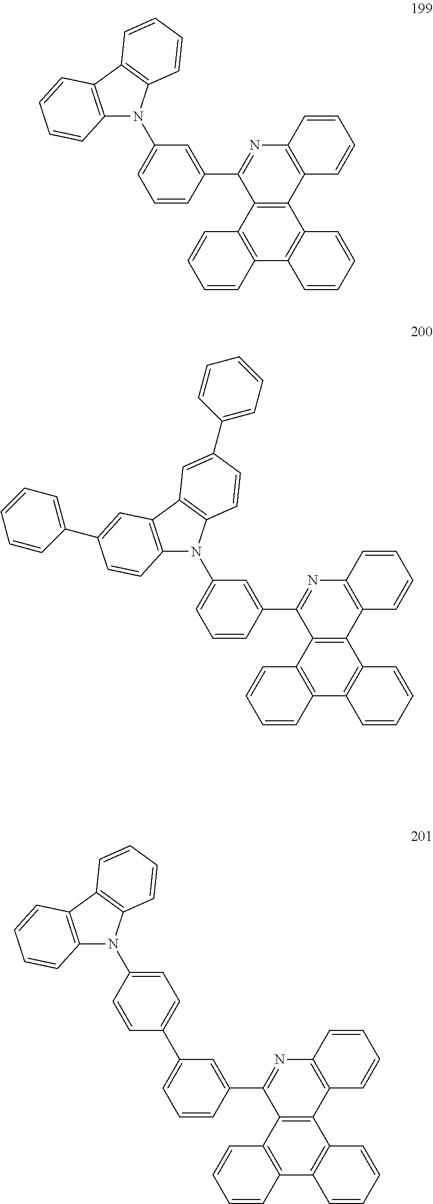 [SMILES: c1cc(-c2nc3ccccc3c3c4ccccc4c4ccccc4c23)cc(-n2c3ccccc3c3ccccc32)c1 ; c1ccc(-c2ccc3c(c2)c2cc(-c4ccccc4)ccc2n3-c2cccc(-c3nc4ccccc4c4c5ccccc5c5ccccc5c34)c2)cc1 ; c1cc(-c2ccc(-n3c4ccccc4c4ccccc43)cc2)cc(-c2nc3ccccc3c3c4ccccc4c4ccccc4c23)c1]

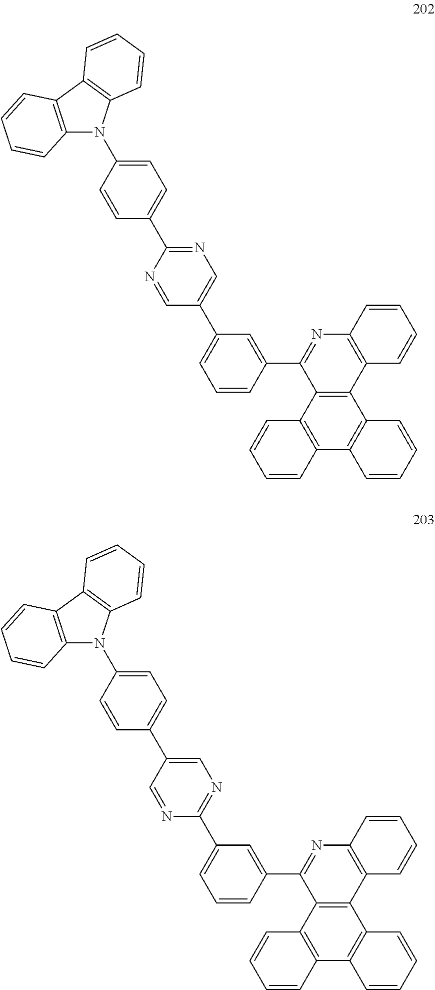 [SMILES: c1cc(-c2cnc(-c3ccc(-n4c5ccccc5c5ccccc54)cc3)nc2)cc(-c2nc3ccccc3c3c4ccccc4c4ccccc4c23)c1 ; c1cc(-c2ncc(-c3ccc(-n4c5ccccc5c5ccccc54)cc3)cn2)cc(-c2nc3ccccc3c3c4ccccc4c4ccccc4c23)c1]

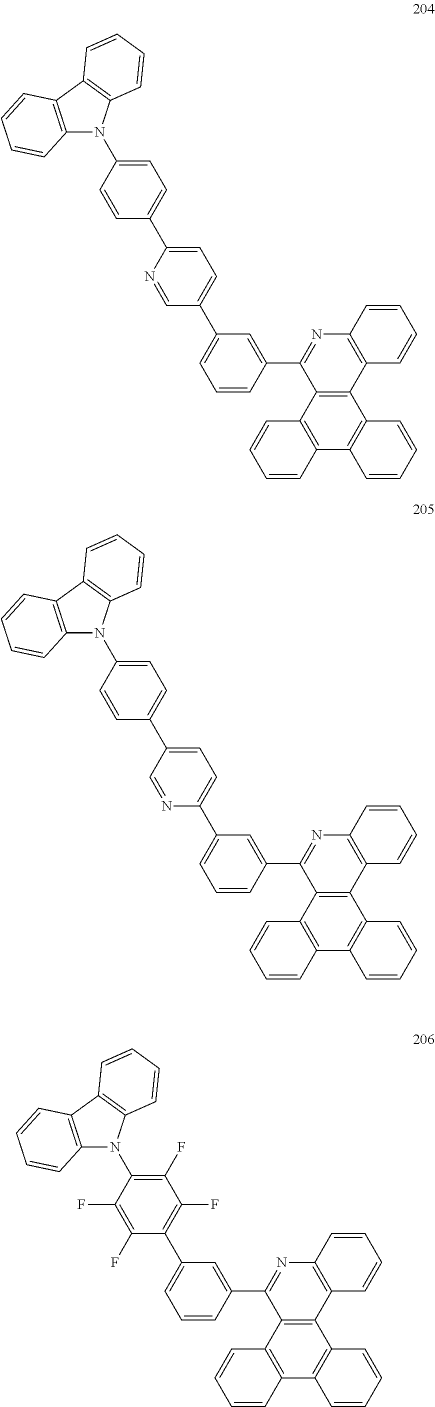 [SMILES: c1cc(-c2ccc(-c3ccc(-n4c5ccccc5c5ccccc54)cc3)nc2)cc(-c2nc3ccccc3c3c4ccccc4c4ccccc4c23)c1 ; c1cc(-c2ccc(-c3ccc(-n4c5ccccc5c5ccccc54)cc3)cn2)cc(-c2nc3ccccc3c3c4ccccc4c4ccccc4c23)c1 ; Fc1c(F)c(-n2c3ccccc3c3ccccc32)c(F)c(F)c1-c1cccc(-c2nc3ccccc3c3c4ccccc4c4ccccc4c23)c1]

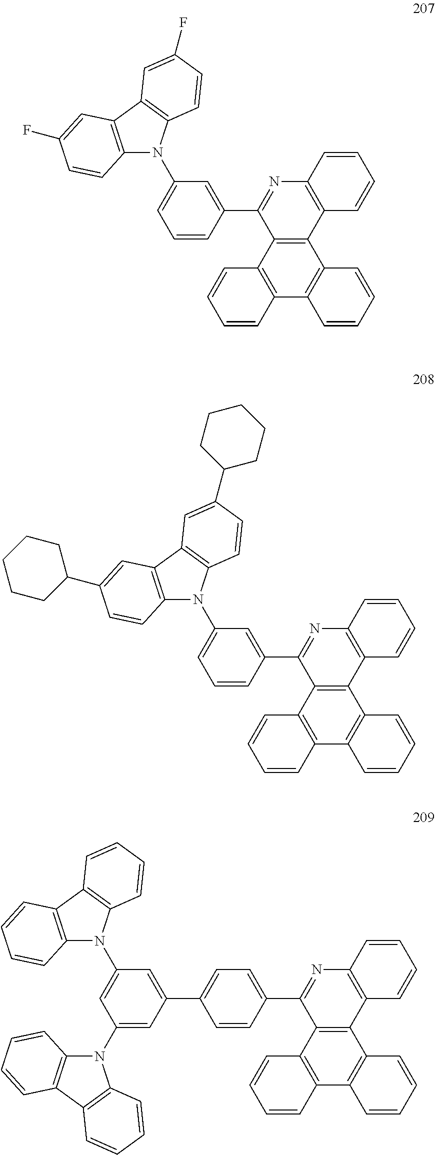 [SMILES: Fc1ccc2c(c1)c1cc(F)ccc1n2-c1cccc(-c2nc3ccccc3c3c4ccccc4c4ccccc4c23)c1 ; c1cc(-c2nc3ccccc3c3c4ccccc4c4ccccc4c23)cc(-n2c3ccc(C4CCCCC4)cc3c3cc(C4CCCCC4)ccc32)c1 ; c1ccc2c(c1)nc(-c1ccc(-c3cc(-n4c5ccccc5c5ccccc54)cc(-n4c5ccccc5c5ccccc54)c3)cc1)c1c3ccccc3c3ccccc3c21]

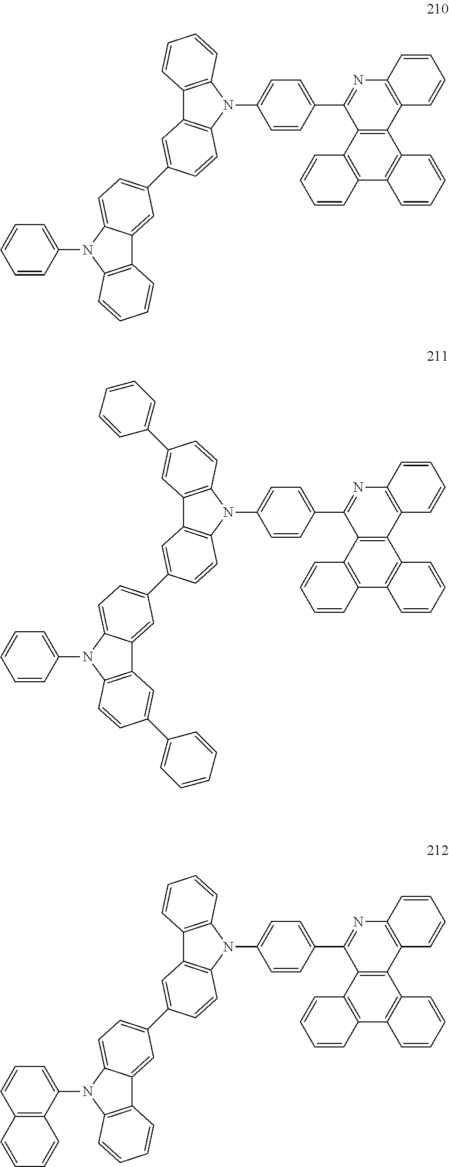 [SMILES: c1ccc(-n2c3ccccc3c3cc(-c4ccc5c(c4)c4ccccc4n5-c4ccc(-c5nc6ccccc6c6c7ccccc7c7ccccc7c56)cc4)ccc32)cc1 ; c1ccc(-c2ccc3c(c2)c2cc(-c4ccc5c(c4)c4cc(-c6ccccc6)ccc4n5-c4ccc(-c5nc6ccccc6c6c7ccccc7c7ccccc7c56)cc4)ccc2n3-c2ccccc2)cc1 ; c1ccc2c(-n3c4ccccc4c4cc(-c5ccc6c(c5)c5ccccc5n6-c5ccc(-c6nc7ccccc7c7c8ccccc8c8ccccc8c67)cc5)ccc43)cccc2c1]

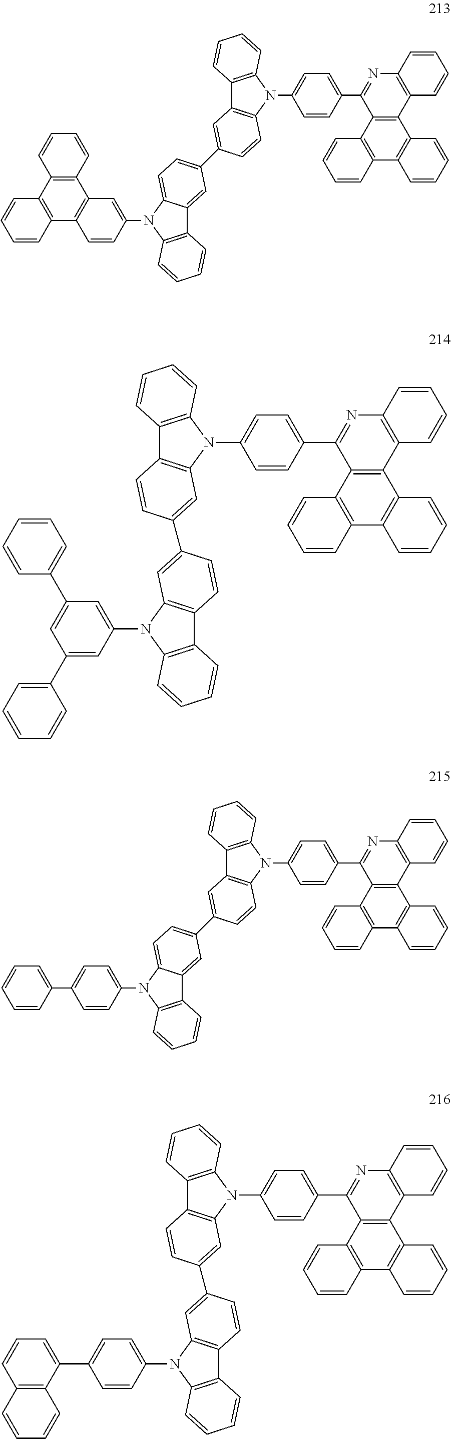 [SMILES: c1ccc2c(c1)nc(-c1ccc(-n3c4ccccc4c4cc(-c5ccc6c(c5)c5ccccc5n6-c5ccc6c7ccccc7c7ccccc7c6c5)ccc43)cc1)c1c3ccccc3c3ccccc3c21 ; c1ccc(-c2cc(-c3ccccc3)cc(-n3c4ccccc4c4ccc(-c5ccc6c7ccccc7n(-c7ccc(-c8nc9ccccc9c9c%10ccccc%10c%10ccccc%10c89)cc7)c6c5)cc43)c2)cc1 ; c1ccc(-c2ccc(-n3c4ccccc4c4cc(-c5ccc6c(c5)c5ccccc5n6-c5ccc(-c6nc7ccccc7c7c8ccccc8c8ccccc8c67)cc5)ccc43)cc2)cc1 ; c1ccc2c(-c3ccc(-n4c5ccccc5c5ccc(-c6ccc7c8ccccc8n(-c8ccc(-c9nc%10ccccc%10c%10c%11ccccc%11c%11ccccc%11c9%10)cc8)c7c6)cc54)cc3)cccc2c1]

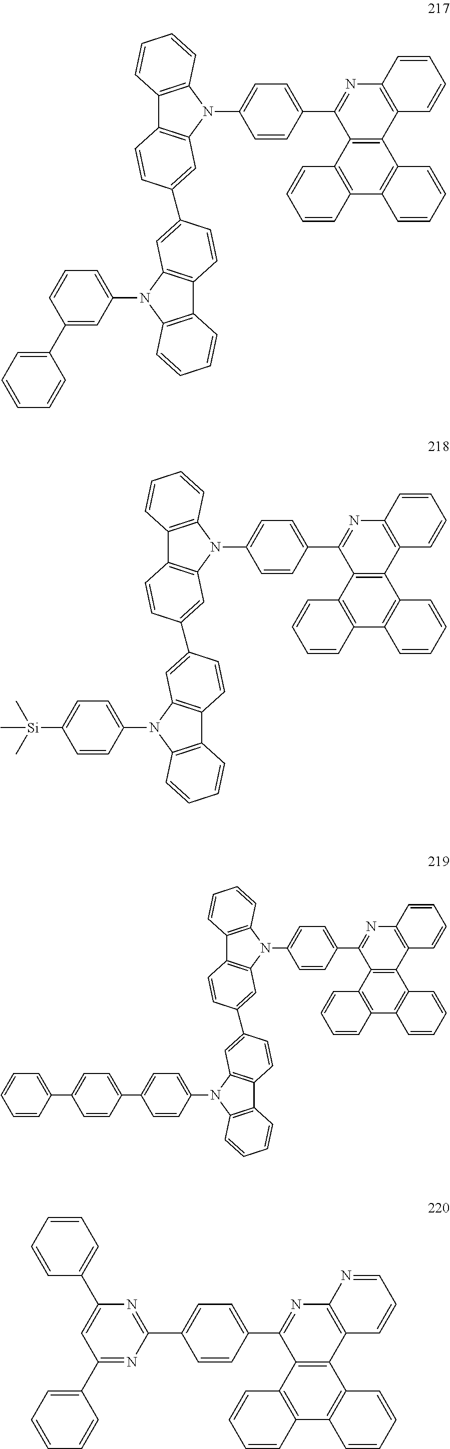 [SMILES: c1ccc(-c2cccc(-n3c4ccccc4c4ccc(-c5ccc6c7ccccc7n(-c7ccc(-c8nc9ccccc9c9c%10ccccc%10c%10ccccc%10c89)cc7)c6c5)cc43)c2)cc1 ; C[Si](C)(C)c1ccc(-n2c3ccccc3c3ccc(-c4ccc5c6ccccc6n(-c6ccc(-c7nc8ccccc8c8c9ccccc9c9ccccc9c78)cc6)c5c4)cc32)cc1 ; c1ccc(-c2ccc(-c3ccc(-n4c5ccccc5c5ccc(-c6ccc7c8ccccc8n(-c8ccc(-c9nc%10ccccc%10c%10c%11ccccc%11c%11ccccc%11c9%10)cc8)c7c6)cc54)cc3)cc2)cc1 ; c1ccc(-c2cc(-c3ccccc3)nc(-c3ccc(-c4nc5ncccc5c5c6ccccc6c6ccccc6c45)cc3)n2)cc1]

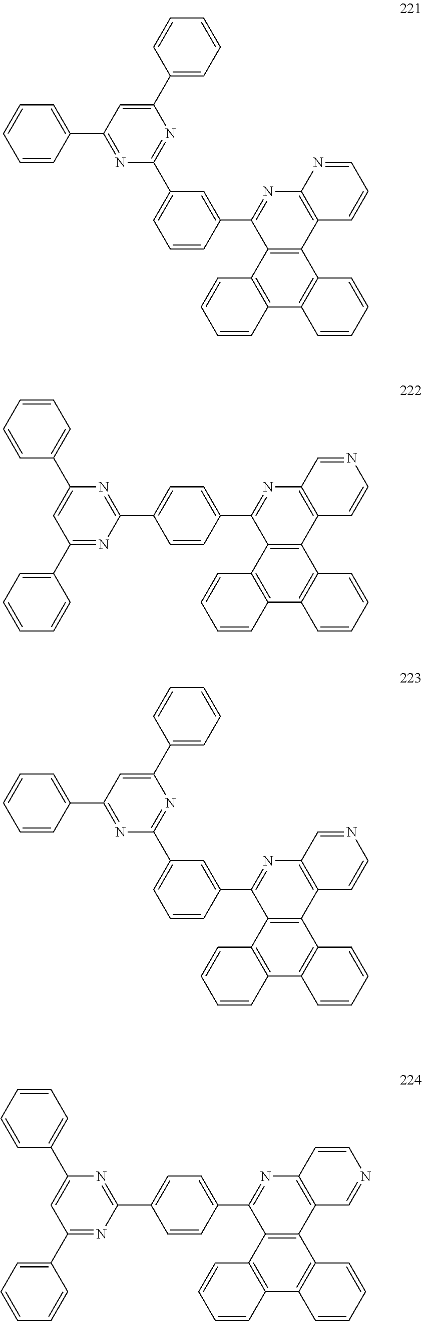 [SMILES: c1ccc(-c2cc(-c3ccccc3)nc(-c3cccc(-c4nc5ncccc5c5c6ccccc6c6ccccc6c45)c3)n2)cc1 ; c1ccc(-c2cc(-c3ccccc3)nc(-c3ccc(-c4nc5cnccc5c5c6ccccc6c6ccccc6c45)cc3)n2)cc1 ; c1ccc(-c2cc(-c3ccccc3)nc(-c3cccc(-c4nc5cnccc5c5c6ccccc6c6ccccc6c45)c3)n2)cc1 ; c1ccc(-c2cc(-c3ccccc3)nc(-c3ccc(-c4nc5ccncc5c5c6ccccc6c6ccccc6c45)cc3)n2)cc1]

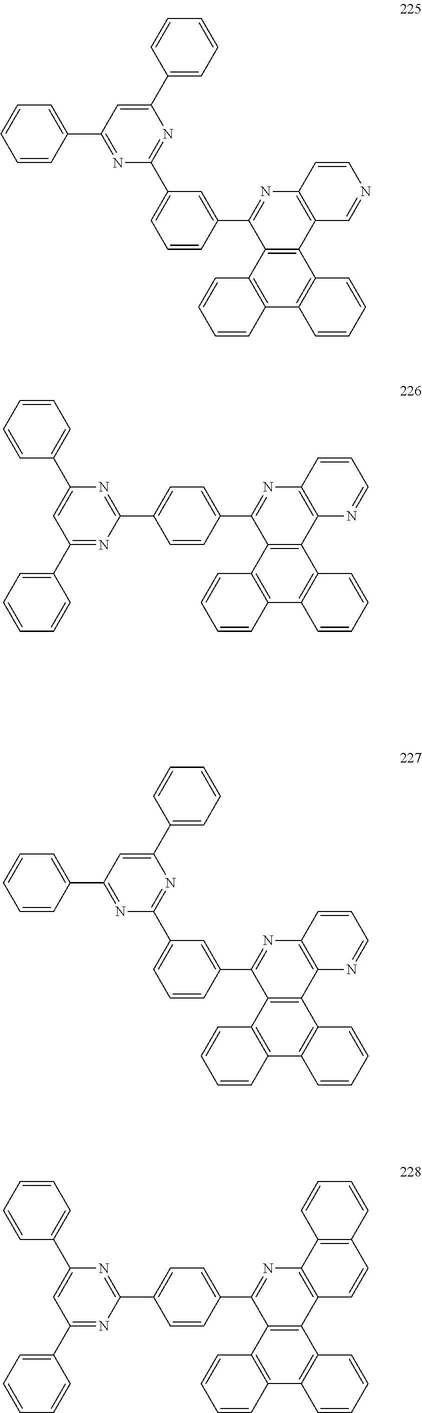 [SMILES: c1ccc(-c2cc(-c3ccccc3)nc(-c3cccc(-c4nc5ccncc5c5c6ccccc6c6ccccc6c45)c3)n2)cc1 ; c1ccc(-c2cc(-c3ccccc3)nc(-c3ccc(-c4nc5cccnc5c5c6ccccc6c6ccccc6c45)cc3)n2)cc1 ; c1ccc(-c2cc(-c3ccccc3)nc(-c3cccc(-c4nc5cccnc5c5c6ccccc6c6ccccc6c45)c3)n2)cc1 ; c1ccc(-c2cc(-c3ccccc3)nc(-c3ccc(-c4nc5c6ccccc6ccc5c5c6ccccc6c6ccccc6c45)cc3)n2)cc1]

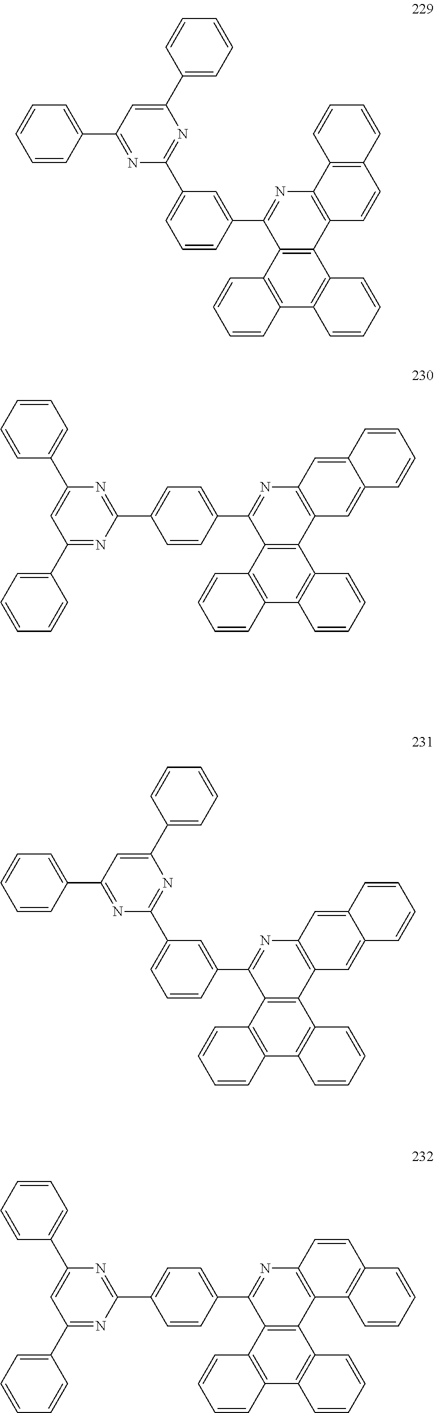 [SMILES: c1ccc(-c2cc(-c3ccccc3)nc(-c3cccc(-c4nc5c6ccccc6ccc5c5c6ccccc6c6ccccc6c45)c3)n2)cc1 ; c1ccc(-c2cc(-c3ccccc3)nc(-c3ccc(-c4nc5cc6ccccc6cc5c5c6ccccc6c6ccccc6c45)cc3)n2)cc1 ; c1ccc(-c2cc(-c3ccccc3)nc(-c3cccc(-c4nc5cc6ccccc6cc5c5c6ccccc6c6ccccc6c45)c3)n2)cc1 ; c1ccc(-c2cc(-c3ccccc3)nc(-c3ccc(-c4nc5ccc6ccccc6c5c5c6ccccc6c6ccccc6c45)cc3)n2)cc1]

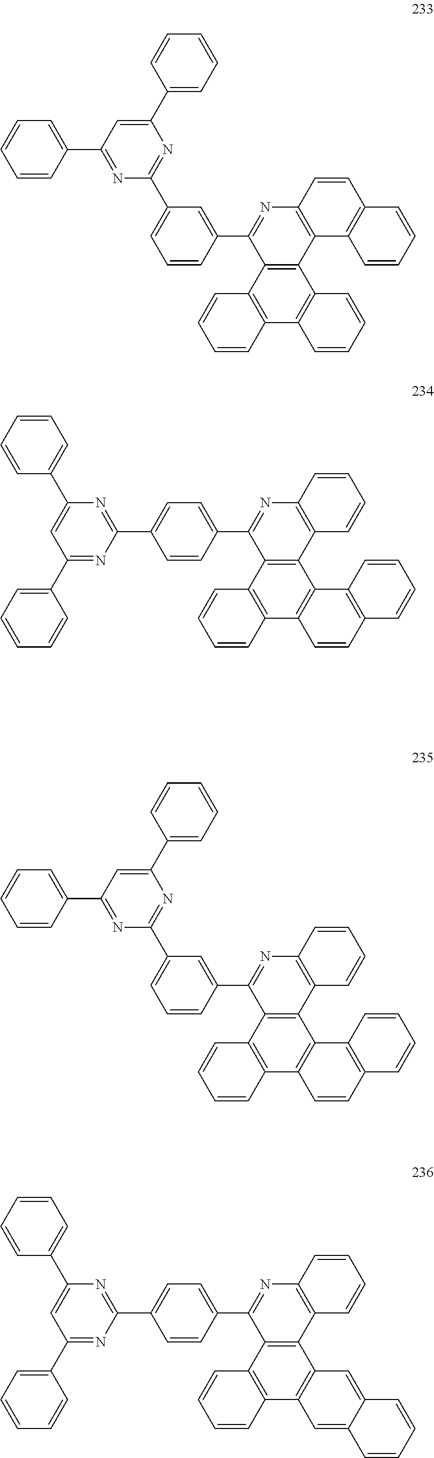 [SMILES: c1ccc(-c2cc(-c3ccccc3)nc(-c3cccc(-c4nc5ccc6ccccc6c5c5c6ccccc6c6ccccc6c45)c3)n2)cc1 ; c1ccc(-c2cc(-c3ccccc3)nc(-c3ccc(-c4nc5ccccc5c5c4c4ccccc4c4ccc6ccccc6c45)cc3)n2)cc1 ; c1ccc(-c2cc(-c3ccccc3)nc(-c3cccc(-c4nc5ccccc5c5c4c4ccccc4c4ccc6ccccc6c45)c3)n2)cc1 ; c1ccc(-c2cc(-c3ccccc3)nc(-c3ccc(-c4nc5ccccc5c5c6cc7ccccc7cc6c6ccccc6c45)cc3)n2)cc1]

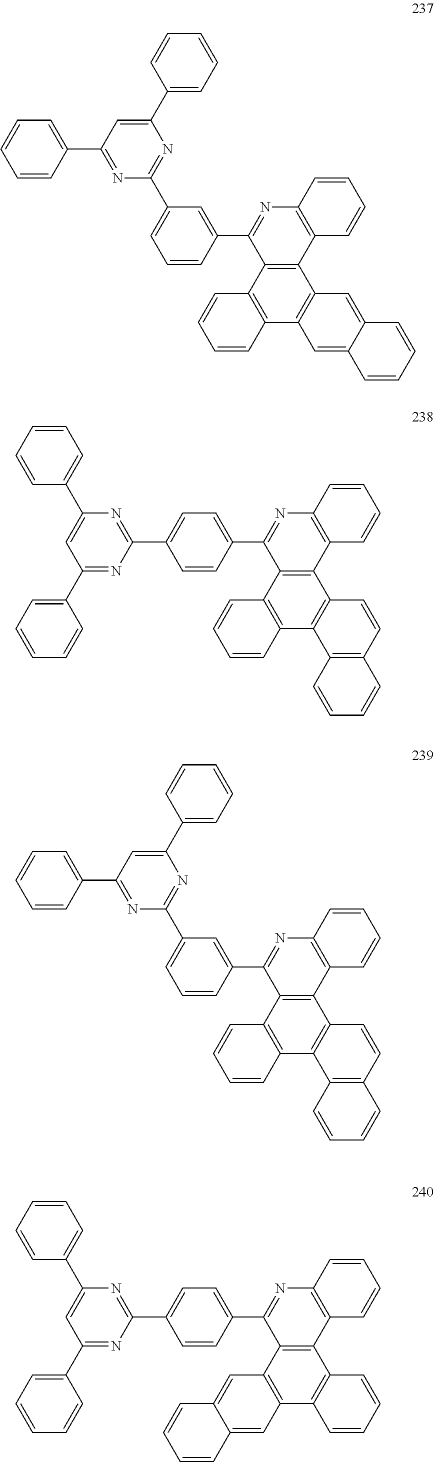 [SMILES: c1ccc(-c2cc(-c3ccccc3)nc(-c3cccc(-c4nc5ccccc5c5c6cc7ccccc7cc6c6ccccc6c45)c3)n2)cc1 ; c1ccc(-c2cc(-c3ccccc3)nc(-c3ccc(-c4nc5ccccc5c5c6ccc7ccccc7c6c6ccccc6c45)cc3)n2)cc1 ; c1ccc(-c2cc(-c3ccccc3)nc(-c3cccc(-c4nc5ccccc5c5c6ccc7ccccc7c6c6ccccc6c45)c3)n2)cc1 ; c1ccc(-c2cc(-c3ccccc3)nc(-c3ccc(-c4nc5ccccc5c5c6ccccc6c6cc7ccccc7cc6c45)cc3)n2)cc1]

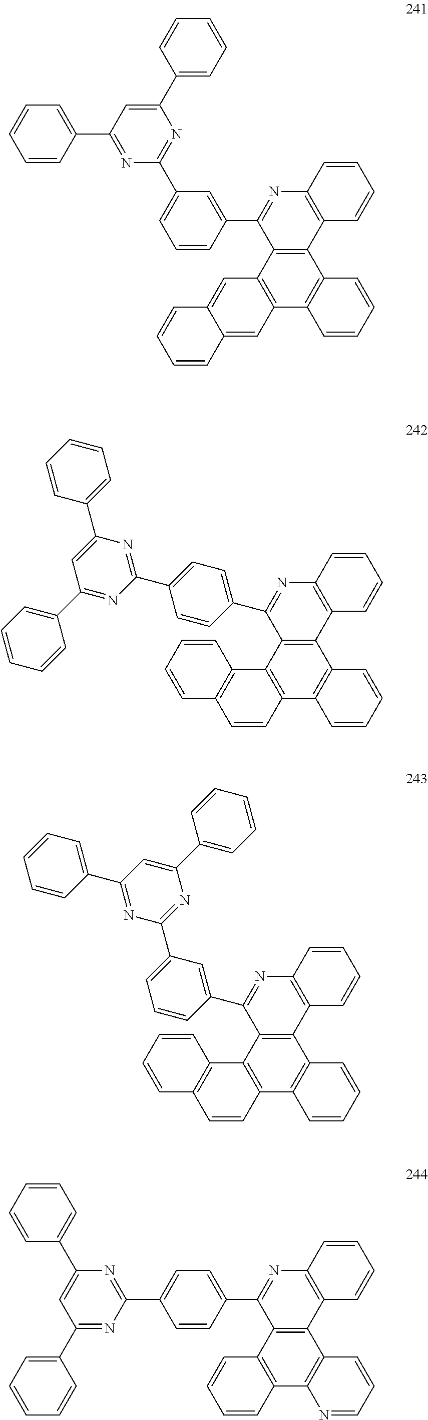 [SMILES: c1ccc(-c2cc(-c3ccccc3)nc(-c3cccc(-c4nc5ccccc5c5c6ccccc6c6cc7ccccc7cc6c45)c3)n2)cc1 ; c1ccc(-c2cc(-c3ccccc3)nc(-c3ccc(-c4nc5ccccc5c5c6ccccc6c6ccc7ccccc7c6c45)cc3)n2)cc1 ; c1ccc(-c2cc(-c3ccccc3)nc(-c3cccc(-c4nc5ccccc5c5c6ccccc6c6ccc7ccccc7c6c45)c3)n2)cc1 ; c1ccc(-c2cc(-c3ccccc3)nc(-c3ccc(-c4nc5ccccc5c5c6cccnc6c6ccccc6c45)cc3)n2)cc1]

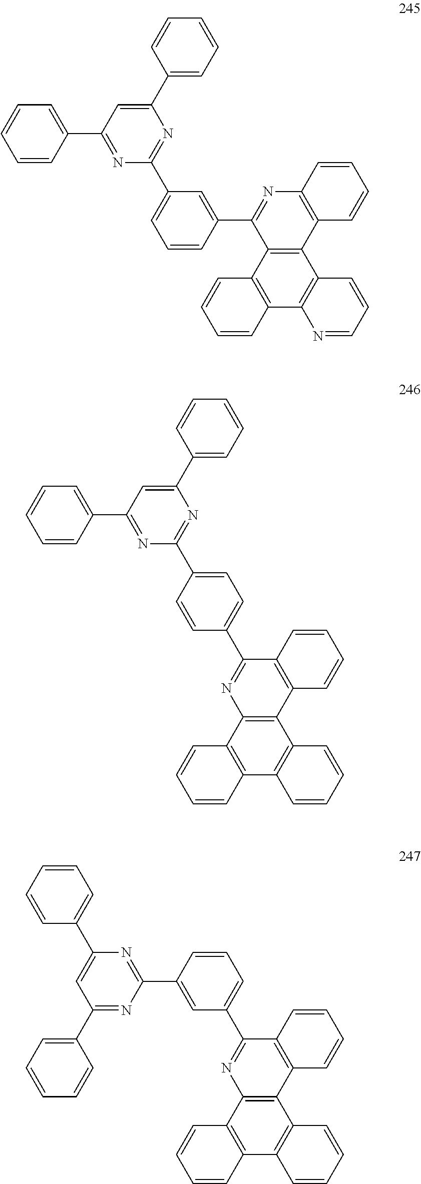 [SMILES: c1ccc(-c2cc(-c3ccccc3)nc(-c3cccc(-c4nc5ccccc5c5c6cccnc6c6ccccc6c45)c3)n2)cc1 ; c1ccc(-c2cc(-c3ccccc3)nc(-c3ccc(-c4nc5c6ccccc6c6ccccc6c5c5ccccc45)cc3)n2)cc1 ; c1ccc(-c2cc(-c3ccccc3)nc(-c3cccc(-c4nc5c6ccccc6c6ccccc6c5c5ccccc45)c3)n2)cc1]

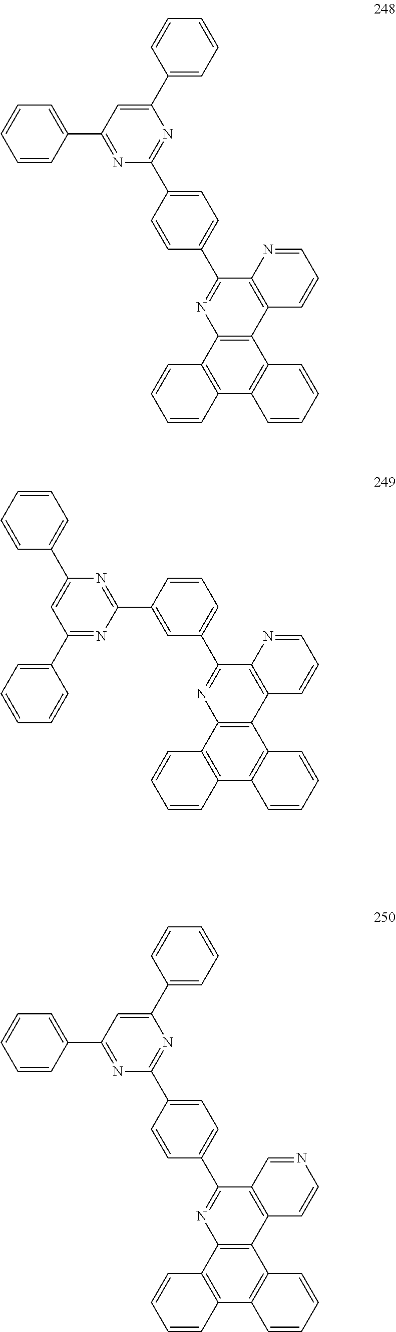 [SMILES: c1ccc(-c2cc(-c3ccccc3)nc(-c3ccc(-c4nc5c6ccccc6c6ccccc6c5c5cccnc45)cc3)n2)cc1 ; c1ccc(-c2cc(-c3ccccc3)nc(-c3cccc(-c4nc5c6ccccc6c6ccccc6c5c5cccnc45)c3)n2)cc1 ; c1ccc(-c2cc(-c3ccccc3)nc(-c3ccc(-c4nc5c6ccccc6c6ccccc6c5c5ccncc45)cc3)n2)cc1]

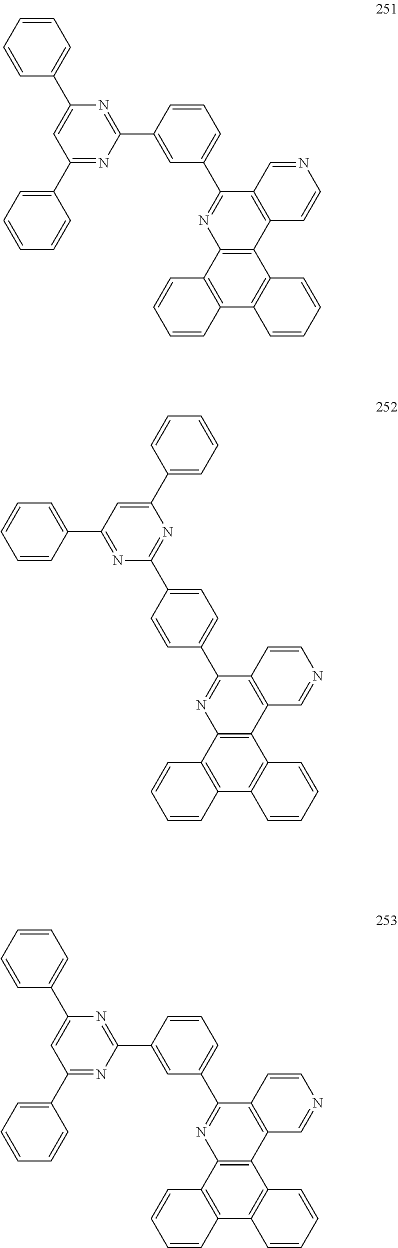 [SMILES: c1ccc(-c2cc(-c3ccccc3)nc(-c3cccc(-c4nc5c6ccccc6c6ccccc6c5c5ccncc45)c3)n2)cc1 ; c1ccc(-c2cc(-c3ccccc3)nc(-c3ccc(-c4nc5c6ccccc6c6ccccc6c5c5cnccc45)cc3)n2)cc1 ; c1ccc(-c2cc(-c3ccccc3)nc(-c3cccc(-c4nc5c6ccccc6c6ccccc6c5c5cnccc45)c3)n2)cc1]

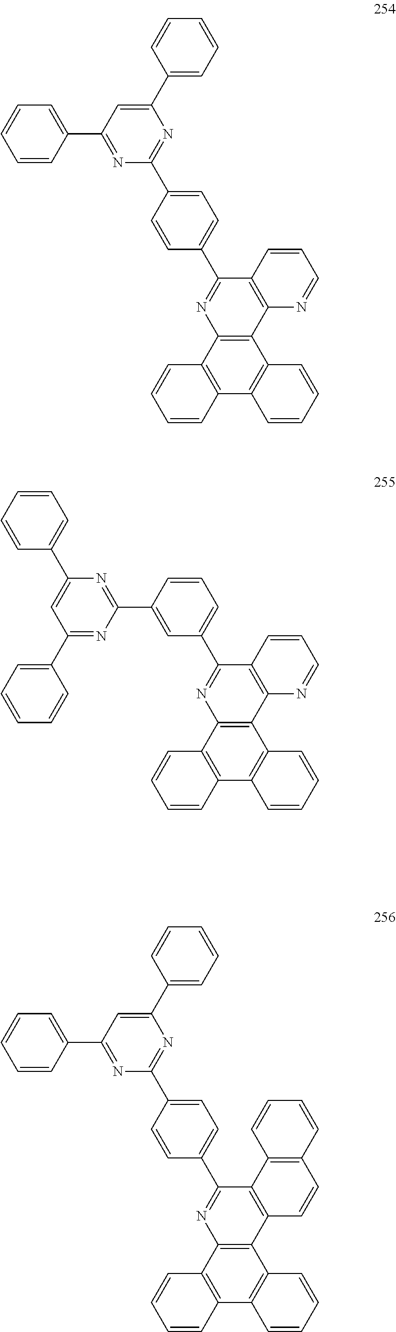 [SMILES: c1ccc(-c2cc(-c3ccccc3)nc(-c3ccc(-c4nc5c6ccccc6c6ccccc6c5c5ncccc45)cc3)n2)cc1 ; c1ccc(-c2cc(-c3ccccc3)nc(-c3cccc(-c4nc5c6ccccc6c6ccccc6c5c5ncccc45)c3)n2)cc1 ; c1ccc(-c2cc(-c3ccccc3)nc(-c3ccc(-c4nc5c6ccccc6c6ccccc6c5c5ccc6ccccc6c45)cc3)n2)cc1]

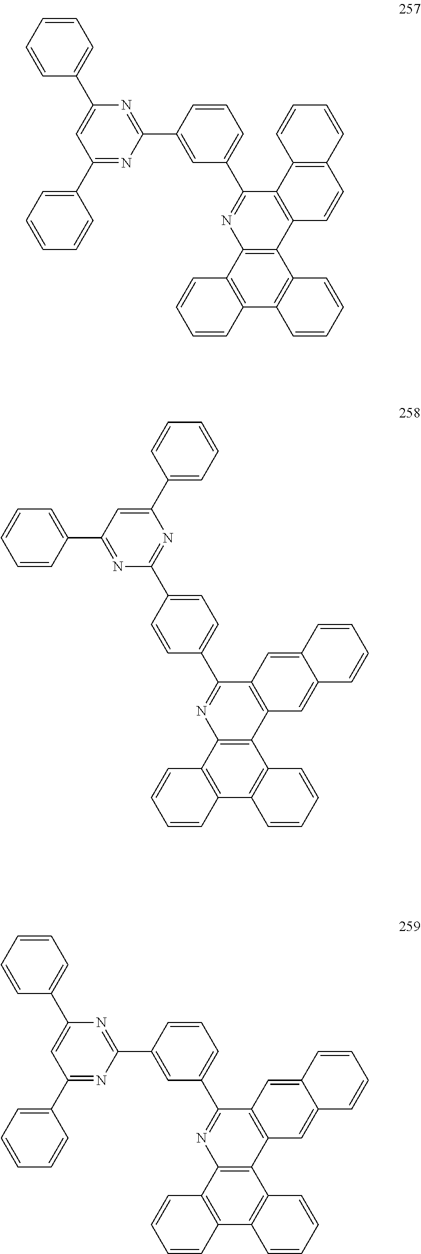 [SMILES: c1ccc(-c2cc(-c3ccccc3)nc(-c3cccc(-c4nc5c6ccccc6c6ccccc6c5c5ccc6ccccc6c45)c3)n2)cc1 ; c1ccc(-c2cc(-c3ccccc3)nc(-c3ccc(-c4nc5c6ccccc6c6ccccc6c5c5cc6ccccc6cc45)cc3)n2)cc1 ; c1ccc(-c2cc(-c3ccccc3)nc(-c3cccc(-c4nc5c6ccccc6c6ccccc6c5c5cc6ccccc6cc45)c3)n2)cc1]

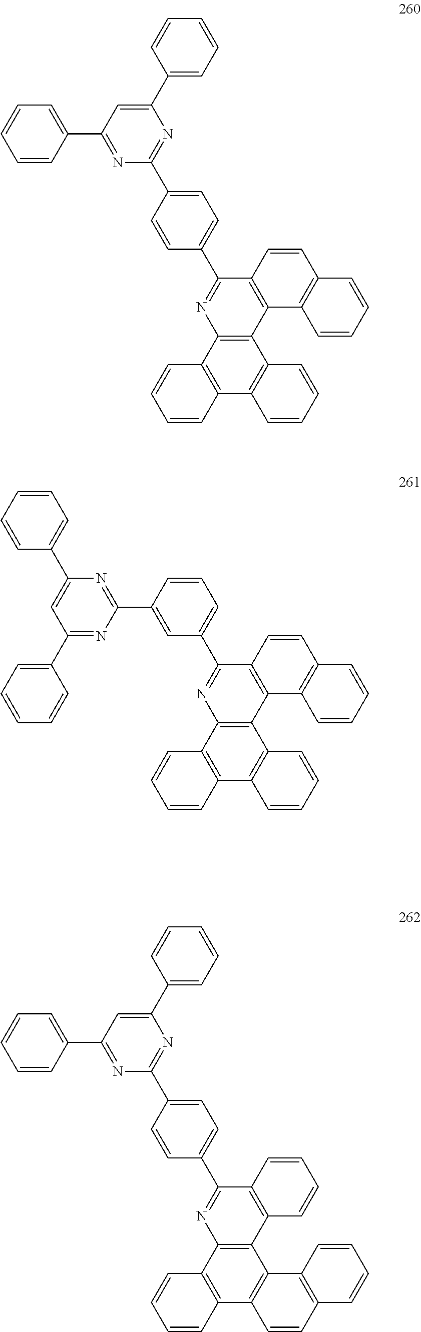 [SMILES: c1ccc(-c2cc(-c3ccccc3)nc(-c3ccc(-c4nc5c6ccccc6c6ccccc6c5c5c4ccc4ccccc45)cc3)n2)cc1 ; c1ccc(-c2cc(-c3ccccc3)nc(-c3cccc(-c4nc5c6ccccc6c6ccccc6c5c5c4ccc4ccccc45)c3)n2)cc1 ; c1ccc(-c2cc(-c3ccccc3)nc(-c3ccc(-c4nc5c6ccccc6c6ccc7ccccc7c6c5c5ccccc45)cc3)n2)cc1]

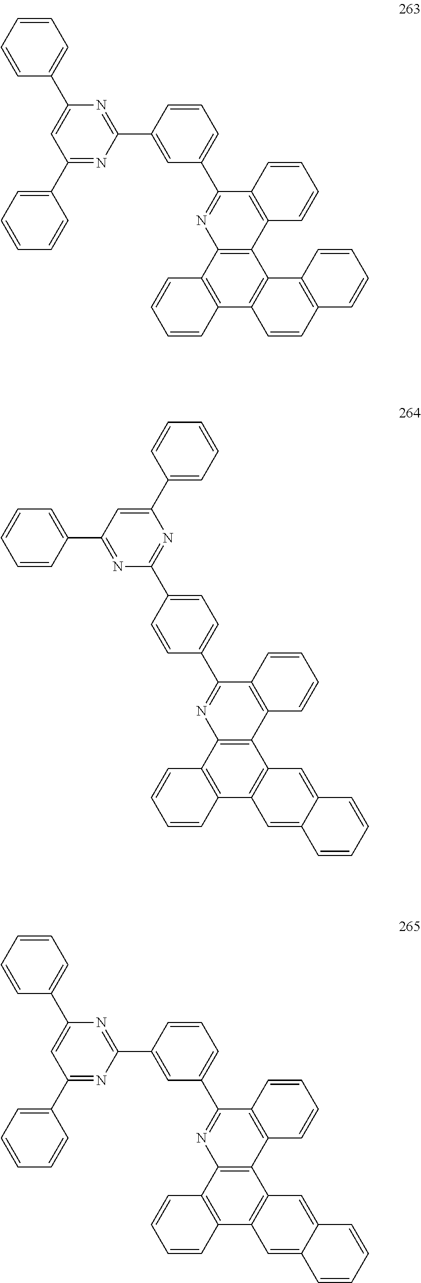 [SMILES: c1ccc(-c2cc(-c3ccccc3)nc(-c3cccc(-c4nc5c6ccccc6c6ccc7ccccc7c6c5c5ccccc45)c3)n2)cc1 ; c1ccc(-c2cc(-c3ccccc3)nc(-c3ccc(-c4nc5c6ccccc6c6cc7ccccc7cc6c5c5ccccc45)cc3)n2)cc1 ; c1ccc(-c2cc(-c3ccccc3)nc(-c3cccc(-c4nc5c6ccccc6c6cc7ccccc7cc6c5c5ccccc45)c3)n2)cc1]

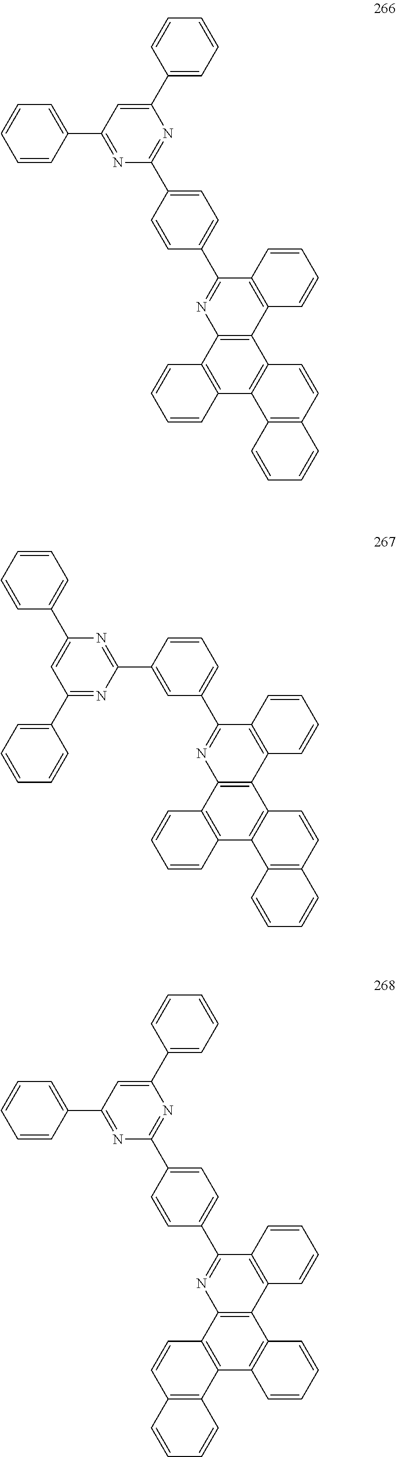 [SMILES: c1ccc(-c2cc(-c3ccccc3)nc(-c3ccc(-c4nc5c6ccccc6c6c7ccccc7ccc6c5c5ccccc45)cc3)n2)cc1 ; c1ccc(-c2cc(-c3ccccc3)nc(-c3cccc(-c4nc5c6ccccc6c6c7ccccc7ccc6c5c5ccccc45)c3)n2)cc1 ; c1ccc(-c2cc(-c3ccccc3)nc(-c3ccc(-c4nc5c6ccc7ccccc7c6c6ccccc6c5c5ccccc45)cc3)n2)cc1]

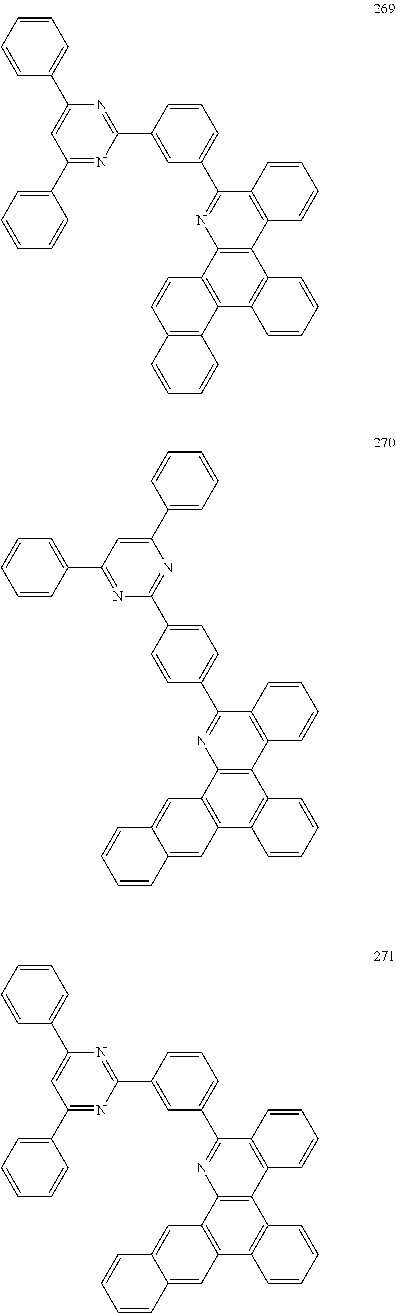 [SMILES: c1ccc(-c2cc(-c3ccccc3)nc(-c3cccc(-c4nc5c6ccc7ccccc7c6c6ccccc6c5c5ccccc45)c3)n2)cc1 ; c1ccc(-c2cc(-c3ccccc3)nc(-c3ccc(-c4nc5c6cc7ccccc7cc6c6ccccc6c5c5ccccc45)cc3)n2)cc1 ; c1ccc(-c2cc(-c3ccccc3)nc(-c3cccc(-c4nc5c6cc7ccccc7cc6c6ccccc6c5c5ccccc45)c3)n2)cc1]

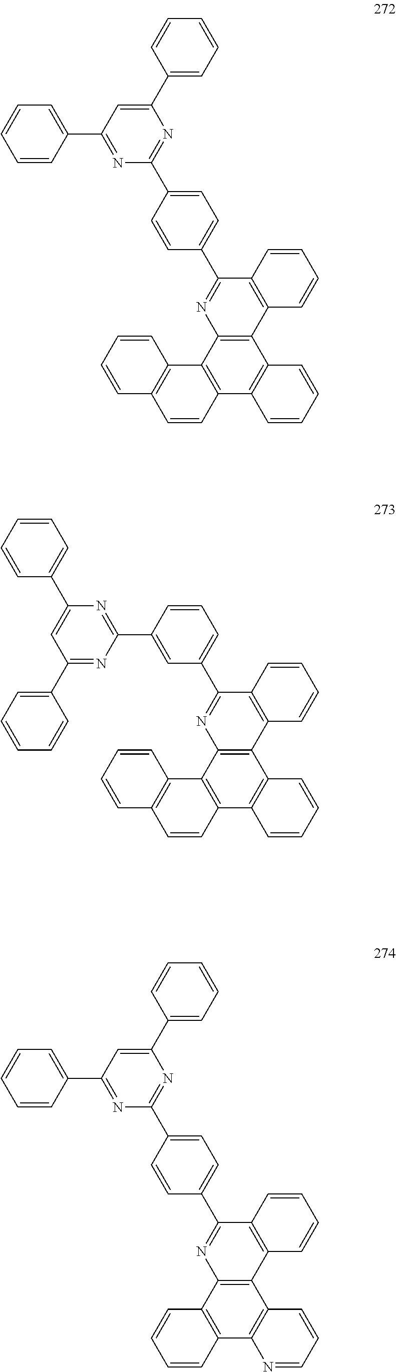 [SMILES: c1ccc(-c2cc(-c3ccccc3)nc(-c3ccc(-c4nc5c6c7ccccc7ccc6c6ccccc6c5c5ccccc45)cc3)n2)cc1 ; c1ccc(-c2cc(-c3ccccc3)nc(-c3cccc(-c4nc5c6c7ccccc7ccc6c6ccccc6c5c5ccccc45)c3)n2)cc1 ; c1ccc(-c2cc(-c3ccccc3)nc(-c3ccc(-c4nc5c6ccccc6c6ncccc6c5c5ccccc45)cc3)n2)cc1]

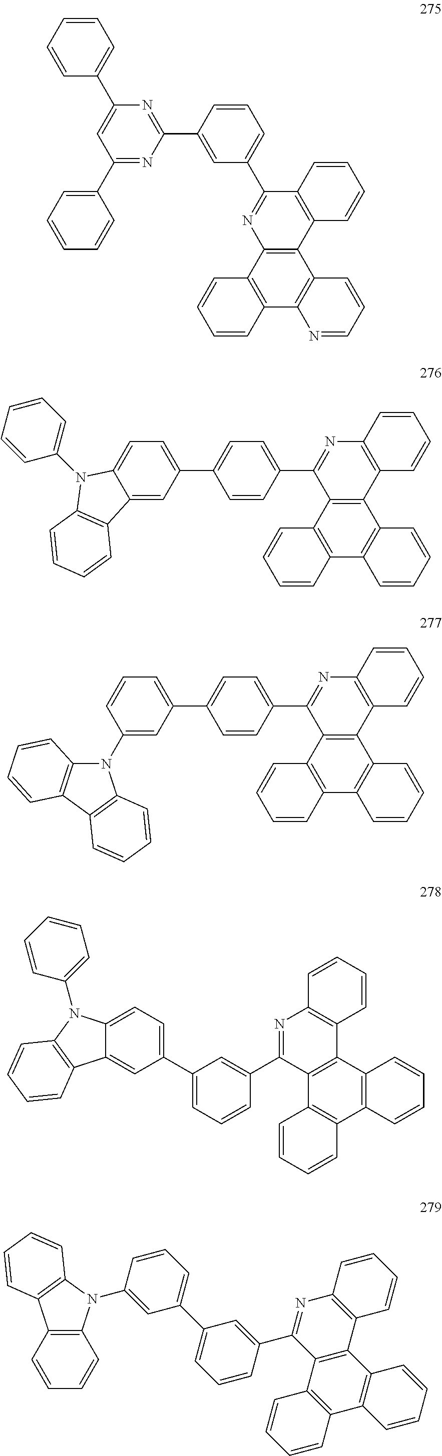 [SMILES: c1ccc(-c2cc(-c3ccccc3)nc(-c3cccc(-c4nc5c6ccccc6c6ncccc6c5c5ccccc45)c3)n2)cc1 ; c1ccc(-n2c3ccccc3c3cc(-c4ccc(-c5nc6ccccc6c6c7ccccc7c7ccccc7c56)cc4)ccc32)cc1 ; c1cc(-c2ccc(-c3nc4ccccc4c4c5ccccc5c5ccccc5c34)cc2)cc(-n2c3ccccc3c3ccccc32)c1 ; c1ccc(-n2c3ccccc3c3cc(-c4cccc(-c5nc6ccccc6c6c7ccccc7c7ccccc7c56)c4)ccc32)cc1 ; c1cc(-c2cccc(-n3c4ccccc4c4ccccc43)c2)cc(-c2nc3ccccc3c3c4ccccc4c4ccccc4c23)c1]

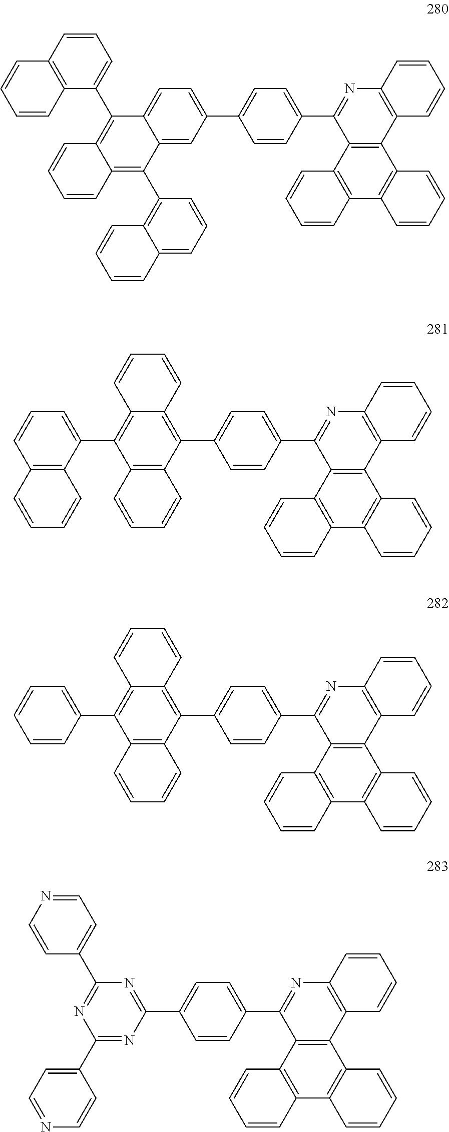 [SMILES: c1ccc2c(-c3c4ccccc4c(-c4cccc5ccccc45)c4cc(-c5ccc(-c6nc7ccccc7c7c8ccccc8c8ccccc8c67)cc5)ccc34)cccc2c1 ; c1ccc2c(-c3c4ccccc4c(-c4ccc(-c5nc6ccccc6c6c7ccccc7c7ccccc7c56)cc4)c4ccccc34)cccc2c1 ; c1ccc(-c2c3ccccc3c(-c3ccc(-c4nc5ccccc5c5c6ccccc6c6ccccc6c45)cc3)c3ccccc23)cc1 ; c1ccc2c(c1)nc(-c1ccc(-c3nc(-c4ccncc4)nc(-c4ccncc4)n3)cc1)c1c3ccccc3c3ccccc3c21]

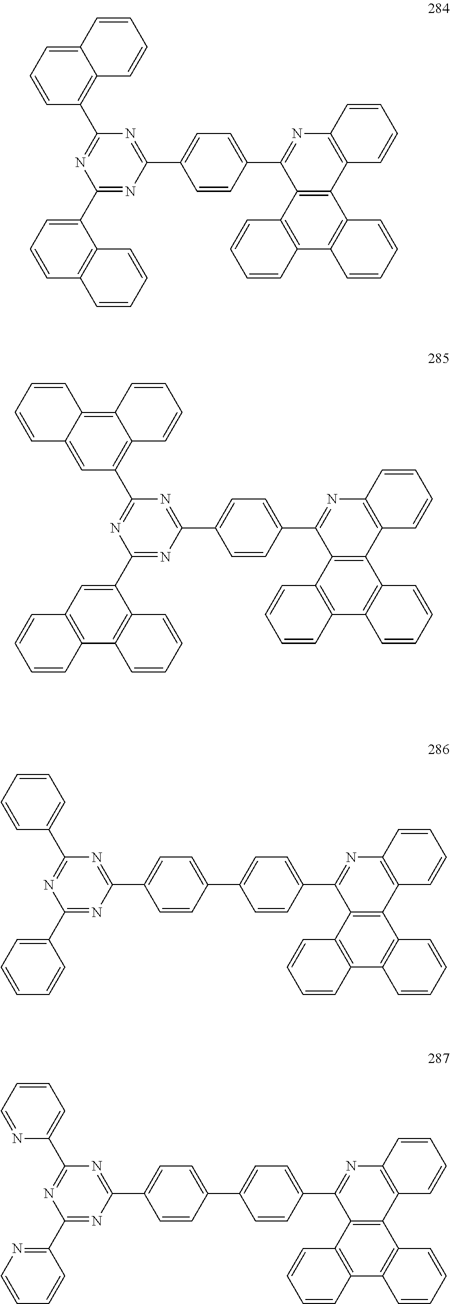 [SMILES: c1ccc2c(-c3nc(-c4ccc(-c5nc6ccccc6c6c7ccccc7c7ccccc7c56)cc4)nc(-c4cccc5ccccc45)n3)cccc2c1 ; c1ccc2c(c1)cc(-c1nc(-c3ccc(-c4nc5ccccc5c5c6ccccc6c6ccccc6c45)cc3)nc(-c3cc4ccccc4c4ccccc34)n1)c1ccccc12 ; c1ccc(-c2nc(-c3ccccc3)nc(-c3ccc(-c4ccc(-c5nc6ccccc6c6c7ccccc7c7ccccc7c56)cc4)cc3)n2)cc1 ; c1ccc(-c2nc(-c3ccc(-c4ccc(-c5nc6ccccc6c6c7ccccc7c7ccccc7c56)cc4)cc3)nc(-c3ccccn3)n2)nc1]

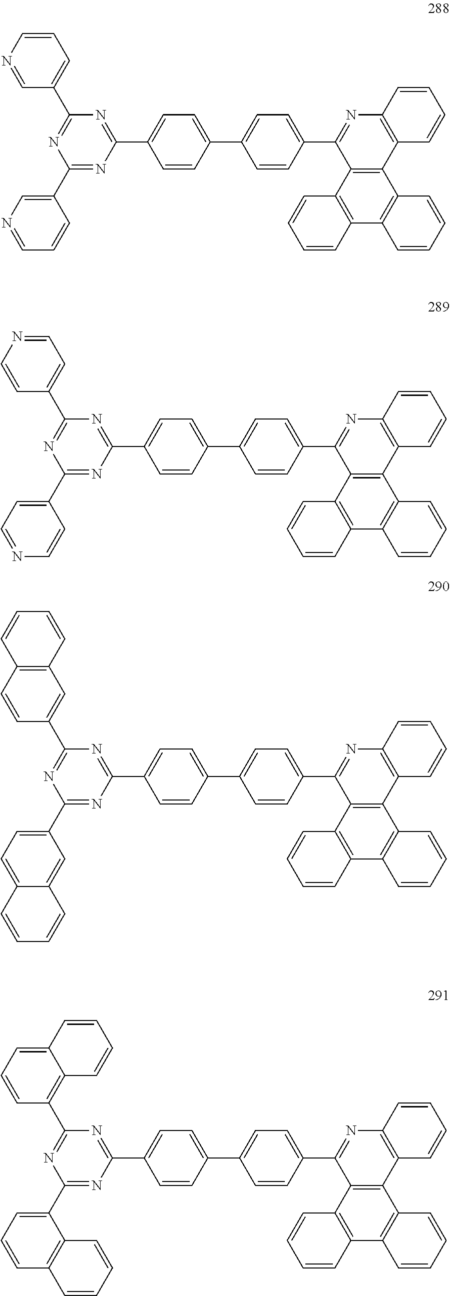 [SMILES: c1cncc(-c2nc(-c3ccc(-c4ccc(-c5nc6ccccc6c6c7ccccc7c7ccccc7c56)cc4)cc3)nc(-c3cccnc3)n2)c1 ; c1ccc2c(c1)nc(-c1ccc(-c3ccc(-c4nc(-c5ccncc5)nc(-c5ccncc5)n4)cc3)cc1)c1c3ccccc3c3ccccc3c21 ; c1ccc2cc(-c3nc(-c4ccc(-c5ccc(-c6nc7ccccc7c7c8ccccc8c8ccccc8c67)cc5)cc4)nc(-c4ccc5ccccc5c4)n3)ccc2c1 ; c1ccc2c(-c3nc(-c4ccc(-c5ccc(-c6nc7ccccc7c7c8ccccc8c8ccccc8c67)cc5)cc4)nc(-c4cccc5ccccc45)n3)cccc2c1]

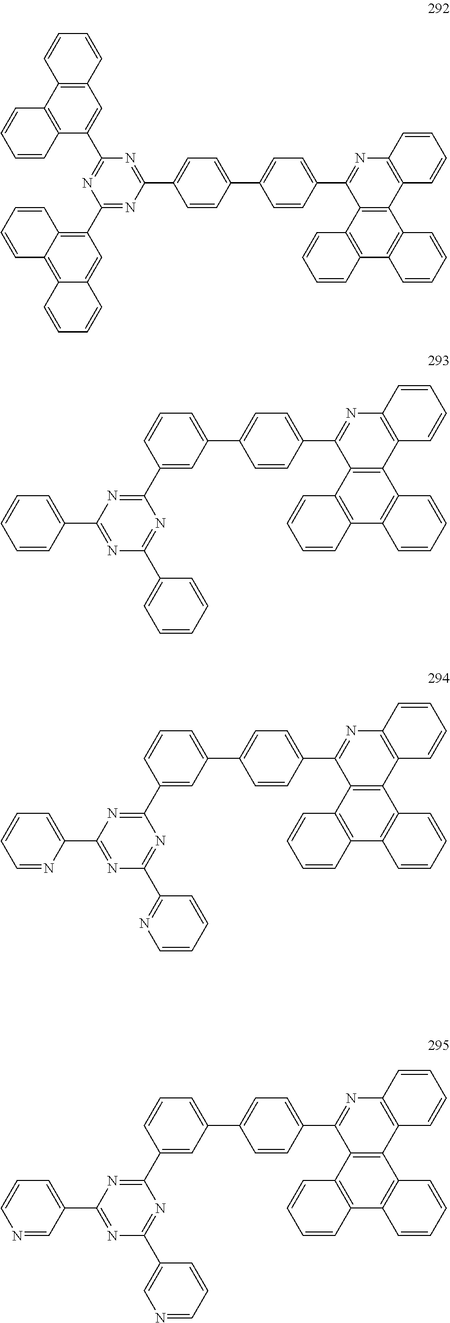 [SMILES: c1ccc2c(c1)cc(-c1nc(-c3ccc(-c4ccc(-c5nc6ccccc6c6c7ccccc7c7ccccc7c56)cc4)cc3)nc(-c3cc4ccccc4c4ccccc34)n1)c1ccccc12 ; c1ccc(-c2nc(-c3ccccc3)nc(-c3cccc(-c4ccc(-c5nc6ccccc6c6c7ccccc7c7ccccc7c56)cc4)c3)n2)cc1 ; c1ccc(-c2nc(-c3cccc(-c4ccc(-c5nc6ccccc6c6c7ccccc7c7ccccc7c56)cc4)c3)nc(-c3ccccn3)n2)nc1 ; c1cncc(-c2nc(-c3cccnc3)nc(-c3cccc(-c4ccc(-c5nc6ccccc6c6c7ccccc7c7ccccc7c56)cc4)c3)n2)c1]

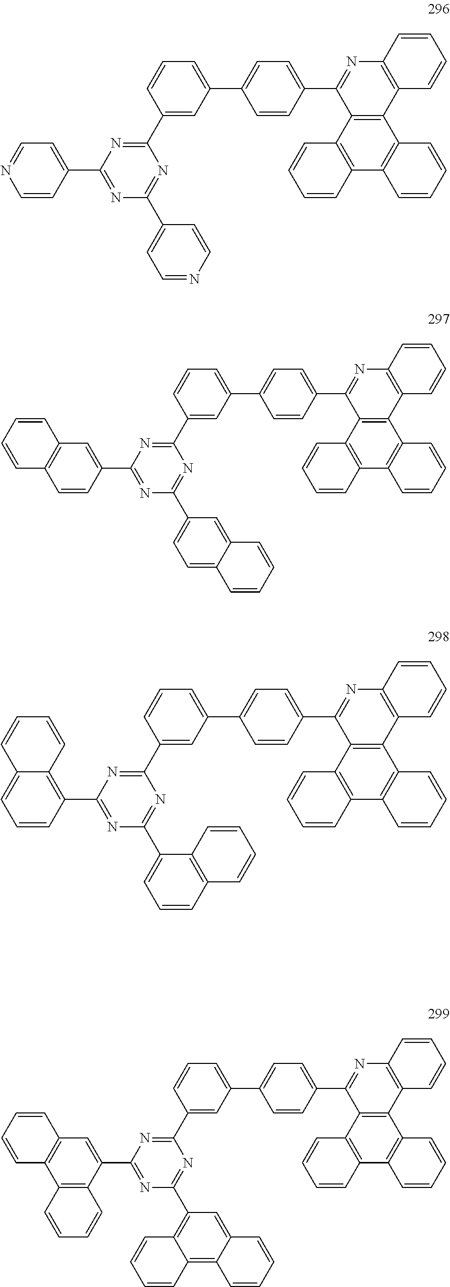 [SMILES: c1cc(-c2ccc(-c3nc4ccccc4c4c5ccccc5c5ccccc5c34)cc2)cc(-c2nc(-c3ccncc3)nc(-c3ccncc3)n2)c1 ; c1cc(-c2ccc(-c3nc4ccccc4c4c5ccccc5c5ccccc5c34)cc2)cc(-c2nc(-c3ccc4ccccc4c3)nc(-c3ccc4ccccc4c3)n2)c1 ; c1cc(-c2ccc(-c3nc4ccccc4c4c5ccccc5c5ccccc5c34)cc2)cc(-c2nc(-c3cccc4ccccc34)nc(-c3cccc4ccccc34)n2)c1 ; c1cc(-c2ccc(-c3nc4ccccc4c4c5ccccc5c5ccccc5c34)cc2)cc(-c2nc(-c3cc4ccccc4c4ccccc34)nc(-c3cc4ccccc4c4ccccc34)n2)c1]

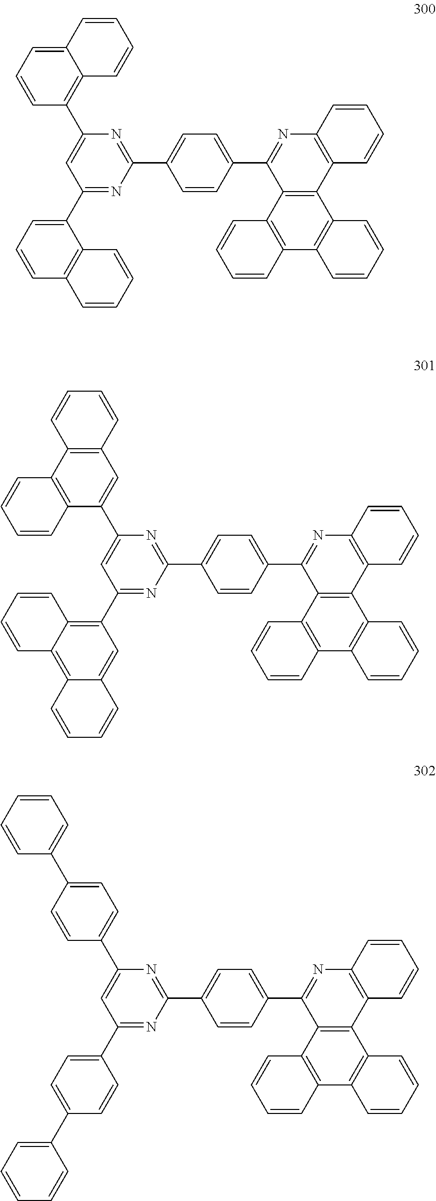 [SMILES: c1ccc2c(-c3cc(-c4cccc5ccccc45)nc(-c4ccc(-c5nc6ccccc6c6c7ccccc7c7ccccc7c56)cc4)n3)cccc2c1 ; c1ccc2c(c1)cc(-c1cc(-c3cc4ccccc4c4ccccc34)nc(-c3ccc(-c4nc5ccccc5c5c6ccccc6c6ccccc6c45)cc3)n1)c1ccccc12 ; c1ccc(-c2ccc(-c3cc(-c4ccc(-c5ccccc5)cc4)nc(-c4ccc(-c5nc6ccccc6c6c7ccccc7c7ccccc7c56)cc4)n3)cc2)cc1]

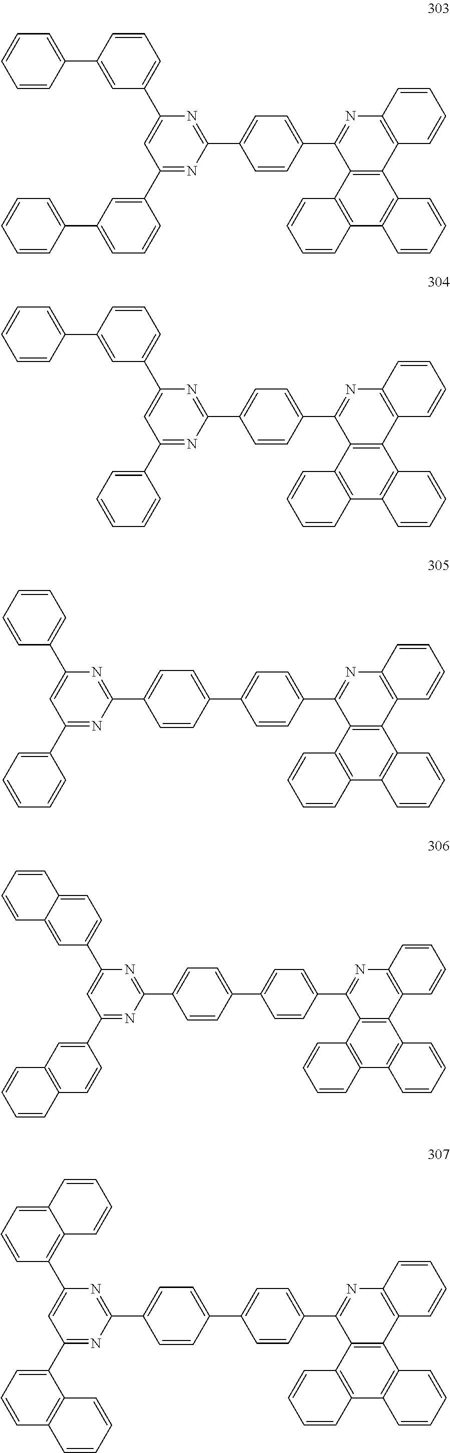 [SMILES: c1ccc(-c2cccc(-c3cc(-c4cccc(-c5ccccc5)c4)nc(-c4ccc(-c5nc6ccccc6c6c7ccccc7c7ccccc7c56)cc4)n3)c2)cc1 ; c1ccc(-c2cccc(-c3cc(-c4ccccc4)nc(-c4ccc(-c5nc6ccccc6c6c7ccccc7c7ccccc7c56)cc4)n3)c2)cc1 ; c1ccc(-c2cc(-c3ccccc3)nc(-c3ccc(-c4ccc(-c5nc6ccccc6c6c7ccccc7c7ccccc7c56)cc4)cc3)n2)cc1 ; c1ccc2cc(-c3cc(-c4ccc5ccccc5c4)nc(-c4ccc(-c5ccc(-c6nc7ccccc7c7c8ccccc8c8ccccc8c67)cc5)cc4)n3)ccc2c1 ; c1ccc2c(-c3cc(-c4cccc5ccccc45)nc(-c4ccc(-c5ccc(-c6nc7ccccc7c7c8ccccc8c8ccccc8c67)cc5)cc4)n3)cccc2c1]

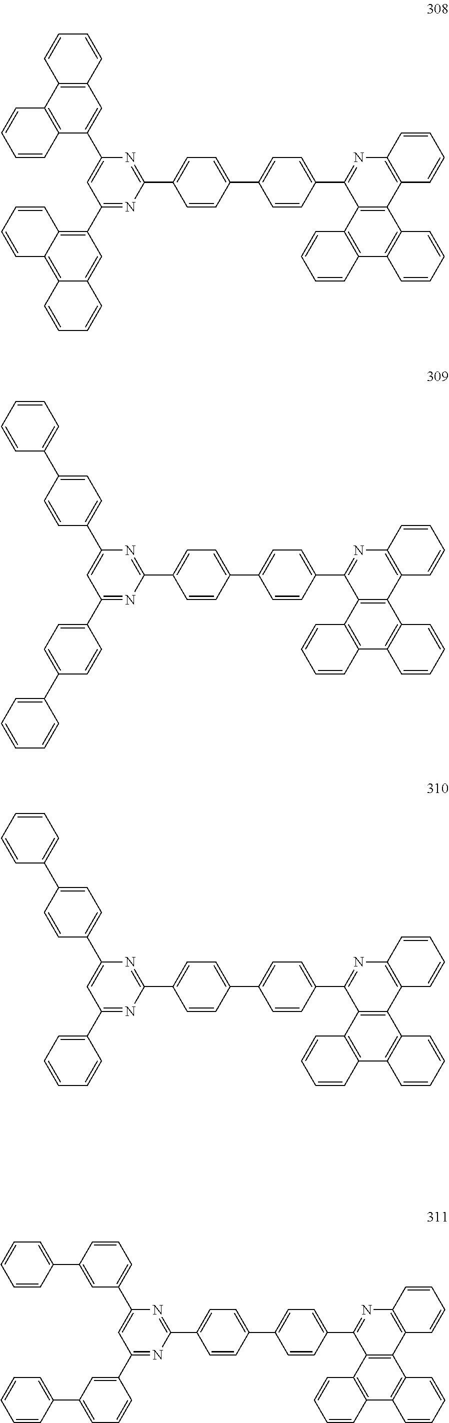 [SMILES: c1ccc2c(c1)cc(-c1cc(-c3cc4ccccc4c4ccccc34)nc(-c3ccc(-c4ccc(-c5nc6ccccc6c6c7ccccc7c7ccccc7c56)cc4)cc3)n1)c1ccccc12 ; c1ccc(-c2ccc(-c3cc(-c4ccc(-c5ccccc5)cc4)nc(-c4ccc(-c5ccc(-c6nc7ccccc7c7c8ccccc8c8ccccc8c67)cc5)cc4)n3)cc2)cc1 ; c1ccc(-c2ccc(-c3cc(-c4ccccc4)nc(-c4ccc(-c5ccc(-c6nc7ccccc7c7c8ccccc8c8ccccc8c67)cc5)cc4)n3)cc2)cc1 ; c1ccc(-c2cccc(-c3cc(-c4cccc(-c5ccccc5)c4)nc(-c4ccc(-c5ccc(-c6nc7ccccc7c7c8ccccc8c8ccccc8c67)cc5)cc4)n3)c2)cc1]

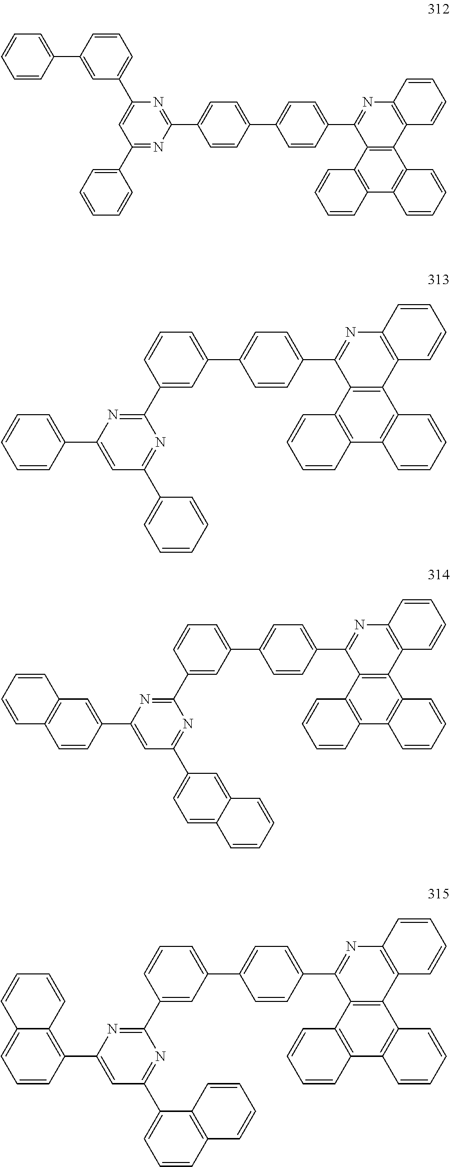 [SMILES: c1ccc(-c2cccc(-c3cc(-c4ccccc4)nc(-c4ccc(-c5ccc(-c6nc7ccccc7c7c8ccccc8c8ccccc8c67)cc5)cc4)n3)c2)cc1 ; c1ccc(-c2cc(-c3ccccc3)nc(-c3cccc(-c4ccc(-c5nc6ccccc6c6c7ccccc7c7ccccc7c56)cc4)c3)n2)cc1 ; c1cc(-c2ccc(-c3nc4ccccc4c4c5ccccc5c5ccccc5c34)cc2)cc(-c2nc(-c3ccc4ccccc4c3)cc(-c3ccc4ccccc4c3)n2)c1 ; c1cc(-c2ccc(-c3nc4ccccc4c4c5ccccc5c5ccccc5c34)cc2)cc(-c2nc(-c3cccc4ccccc34)cc(-c3cccc4ccccc34)n2)c1]

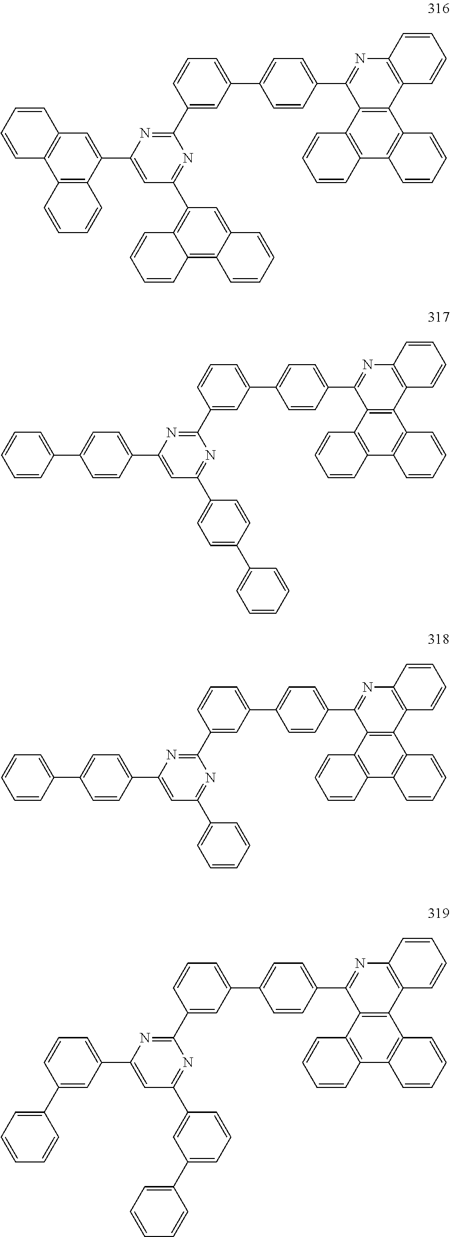 [SMILES: c1cc(-c2ccc(-c3nc4ccccc4c4c5ccccc5c5ccccc5c34)cc2)cc(-c2nc(-c3cc4ccccc4c4ccccc34)cc(-c3cc4ccccc4c4ccccc34)n2)c1 ; c1ccc(-c2ccc(-c3cc(-c4ccc(-c5ccccc5)cc4)nc(-c4cccc(-c5ccc(-c6nc7ccccc7c7c8ccccc8c8ccccc8c67)cc5)c4)n3)cc2)cc1 ; c1ccc(-c2ccc(-c3cc(-c4ccccc4)nc(-c4cccc(-c5ccc(-c6nc7ccccc7c7c8ccccc8c8ccccc8c67)cc5)c4)n3)cc2)cc1 ; c1ccc(-c2cccc(-c3cc(-c4cccc(-c5ccccc5)c4)nc(-c4cccc(-c5ccc(-c6nc7ccccc7c7c8ccccc8c8ccccc8c67)cc5)c4)n3)c2)cc1]

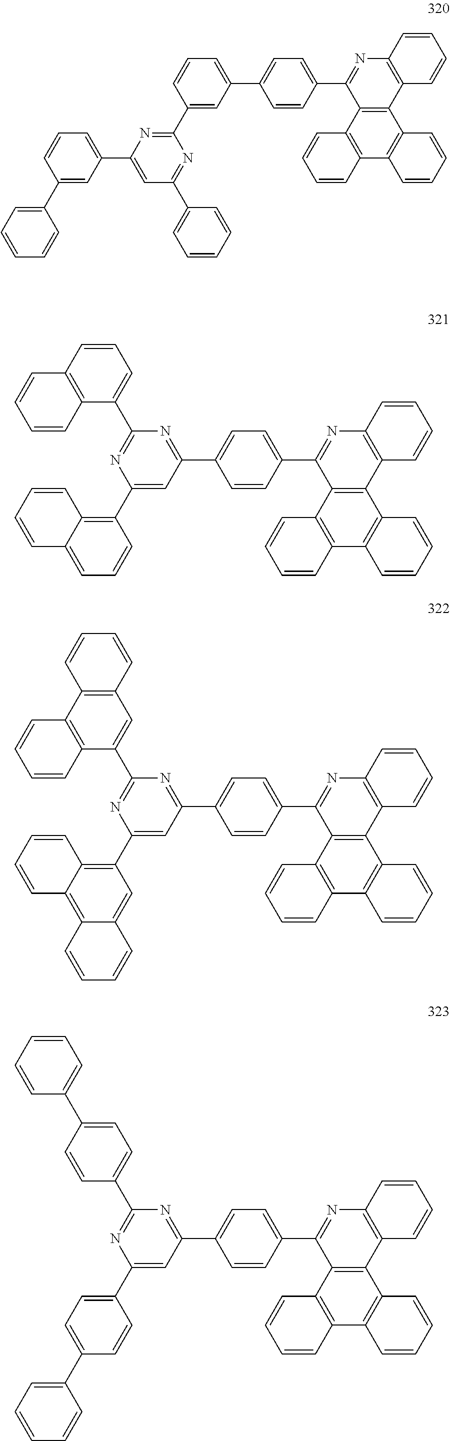 [SMILES: c1ccc(-c2cccc(-c3cc(-c4ccccc4)nc(-c4cccc(-c5ccc(-c6nc7ccccc7c7c8ccccc8c8ccccc8c67)cc5)c4)n3)c2)cc1 ; c1ccc2c(-c3cc(-c4ccc(-c5nc6ccccc6c6c7ccccc7c7ccccc7c56)cc4)nc(-c4cccc5ccccc45)n3)cccc2c1 ; c1ccc2c(c1)cc(-c1cc(-c3ccc(-c4nc5ccccc5c5c6ccccc6c6ccccc6c45)cc3)nc(-c3cc4ccccc4c4ccccc34)n1)c1ccccc12 ; c1ccc(-c2ccc(-c3cc(-c4ccc(-c5nc6ccccc6c6c7ccccc7c7ccccc7c56)cc4)nc(-c4ccc(-c5ccccc5)cc4)n3)cc2)cc1]

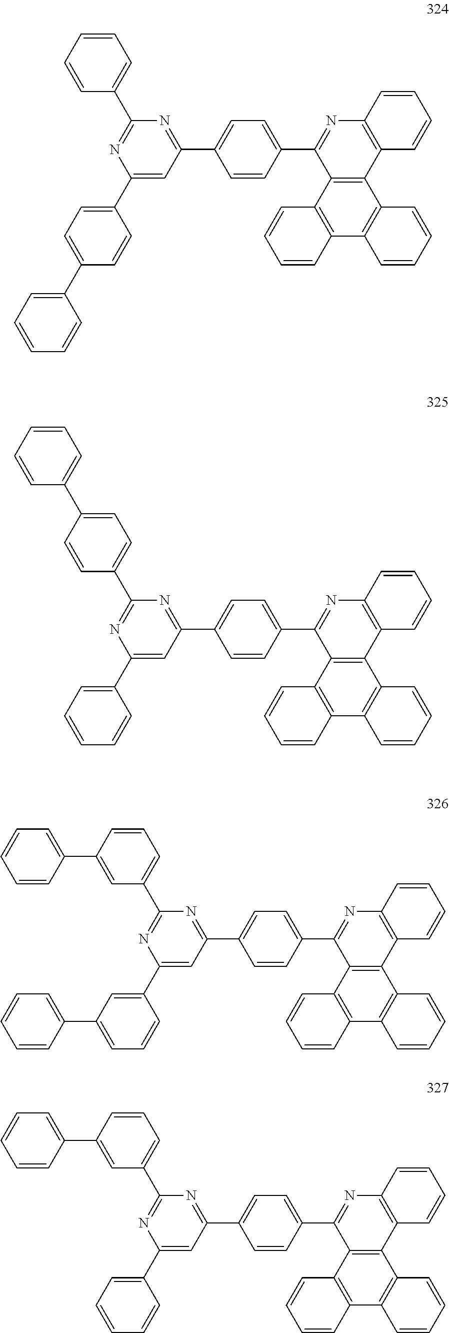 [SMILES: c1ccc(-c2ccc(-c3cc(-c4ccc(-c5nc6ccccc6c6c7ccccc7c7ccccc7c56)cc4)nc(-c4ccccc4)n3)cc2)cc1 ; c1ccc(-c2ccc(-c3nc(-c4ccccc4)cc(-c4ccc(-c5nc6ccccc6c6c7ccccc7c7ccccc7c56)cc4)n3)cc2)cc1 ; c1ccc(-c2cccc(-c3cc(-c4ccc(-c5nc6ccccc6c6c7ccccc7c7ccccc7c56)cc4)nc(-c4cccc(-c5ccccc5)c4)n3)c2)cc1 ; c1ccc(-c2cccc(-c3nc(-c4ccccc4)cc(-c4ccc(-c5nc6ccccc6c6c7ccccc7c7ccccc7c56)cc4)n3)c2)cc1]

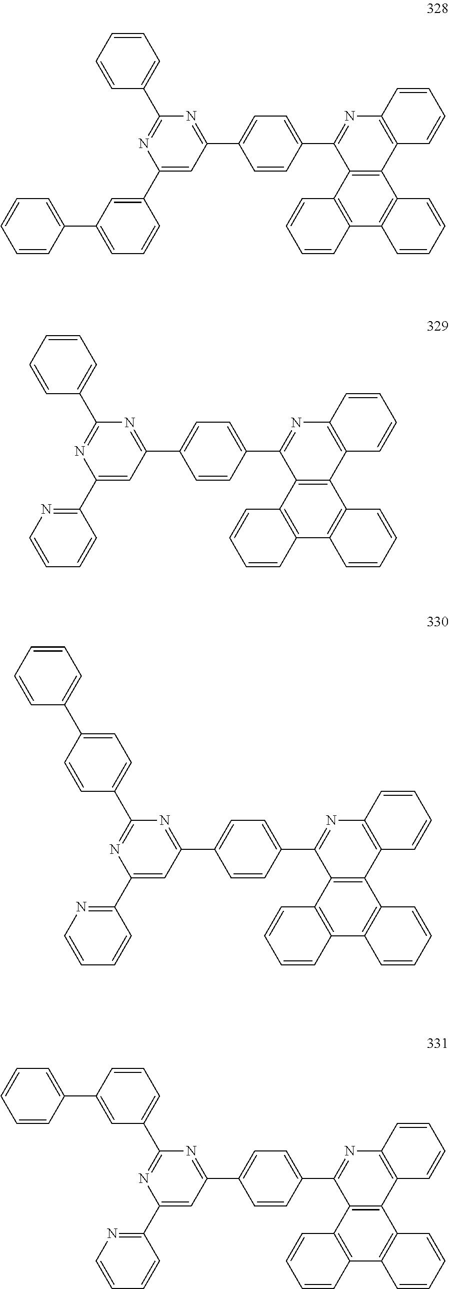 [SMILES: c1ccc(-c2cccc(-c3cc(-c4ccc(-c5nc6ccccc6c6c7ccccc7c7ccccc7c56)cc4)nc(-c4ccccc4)n3)c2)cc1 ; c1ccc(-c2nc(-c3ccc(-c4nc5ccccc5c5c6ccccc6c6ccccc6c45)cc3)cc(-c3ccccn3)n2)cc1 ; c1ccc(-c2ccc(-c3nc(-c4ccc(-c5nc6ccccc6c6c7ccccc7c7ccccc7c56)cc4)cc(-c4ccccn4)n3)cc2)cc1 ; c1ccc(-c2cccc(-c3nc(-c4ccc(-c5nc6ccccc6c6c7ccccc7c7ccccc7c56)cc4)cc(-c4ccccn4)n3)c2)cc1]

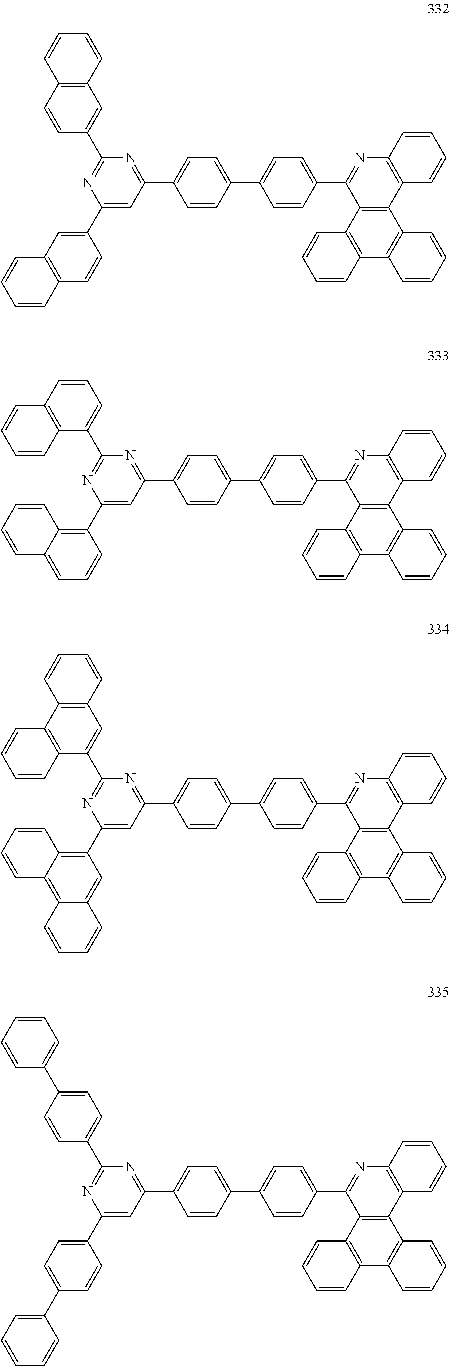 [SMILES: c1ccc2cc(-c3cc(-c4ccc(-c5ccc(-c6nc7ccccc7c7c8ccccc8c8ccccc8c67)cc5)cc4)nc(-c4ccc5ccccc5c4)n3)ccc2c1 ; c1ccc2c(-c3cc(-c4ccc(-c5ccc(-c6nc7ccccc7c7c8ccccc8c8ccccc8c67)cc5)cc4)nc(-c4cccc5ccccc45)n3)cccc2c1 ; c1ccc2c(c1)cc(-c1cc(-c3ccc(-c4ccc(-c5nc6ccccc6c6c7ccccc7c7ccccc7c56)cc4)cc3)nc(-c3cc4ccccc4c4ccccc34)n1)c1ccccc12 ; c1ccc(-c2ccc(-c3cc(-c4ccc(-c5ccc(-c6nc7ccccc7c7c8ccccc8c8ccccc8c67)cc5)cc4)nc(-c4ccc(-c5ccccc5)cc4)n3)cc2)cc1]

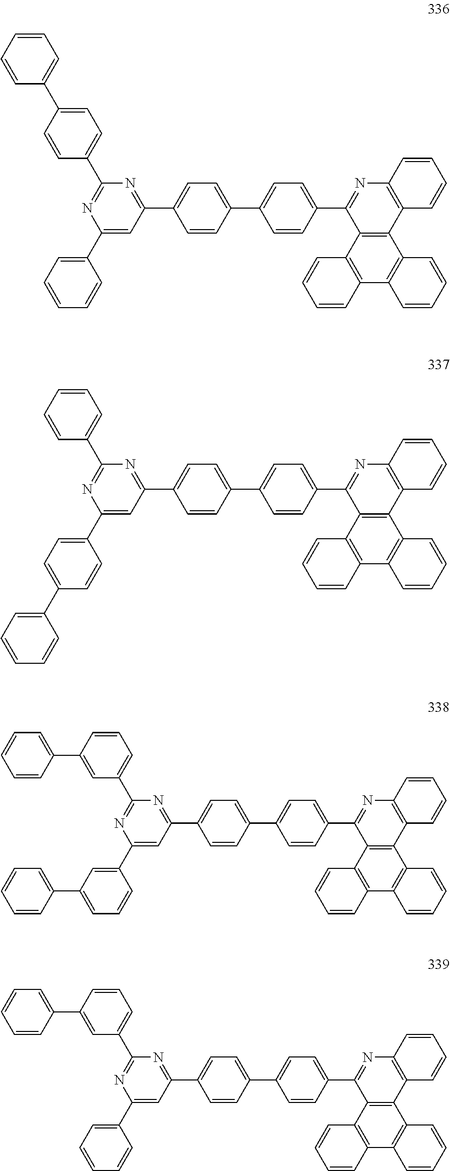 [SMILES: c1ccc(-c2ccc(-c3nc(-c4ccccc4)cc(-c4ccc(-c5ccc(-c6nc7ccccc7c7c8ccccc8c8ccccc8c67)cc5)cc4)n3)cc2)cc1 ; c1ccc(-c2ccc(-c3cc(-c4ccc(-c5ccc(-c6nc7ccccc7c7c8ccccc8c8ccccc8c67)cc5)cc4)nc(-c4ccccc4)n3)cc2)cc1 ; c1ccc(-c2cccc(-c3cc(-c4ccc(-c5ccc(-c6nc7ccccc7c7c8ccccc8c8ccccc8c67)cc5)cc4)nc(-c4cccc(-c5ccccc5)c4)n3)c2)cc1 ; c1ccc(-c2cccc(-c3nc(-c4ccccc4)cc(-c4ccc(-c5ccc(-c6nc7ccccc7c7c8ccccc8c8ccccc8c67)cc5)cc4)n3)c2)cc1]

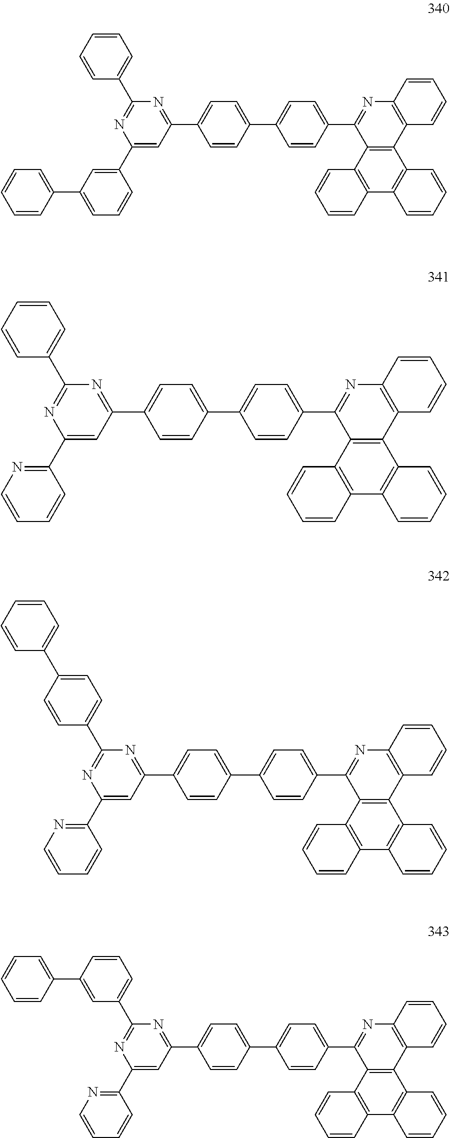 [SMILES: c1ccc(-c2cccc(-c3cc(-c4ccc(-c5ccc(-c6nc7ccccc7c7c8ccccc8c8ccccc8c67)cc5)cc4)nc(-c4ccccc4)n3)c2)cc1 ; c1ccc(-c2nc(-c3ccc(-c4ccc(-c5nc6ccccc6c6c7ccccc7c7ccccc7c56)cc4)cc3)cc(-c3ccccn3)n2)cc1 ; c1ccc(-c2ccc(-c3nc(-c4ccc(-c5ccc(-c6nc7ccccc7c7c8ccccc8c8ccccc8c67)cc5)cc4)cc(-c4ccccn4)n3)cc2)cc1 ; c1ccc(-c2cccc(-c3nc(-c4ccc(-c5ccc(-c6nc7ccccc7c7c8ccccc8c8ccccc8c67)cc5)cc4)cc(-c4ccccn4)n3)c2)cc1]

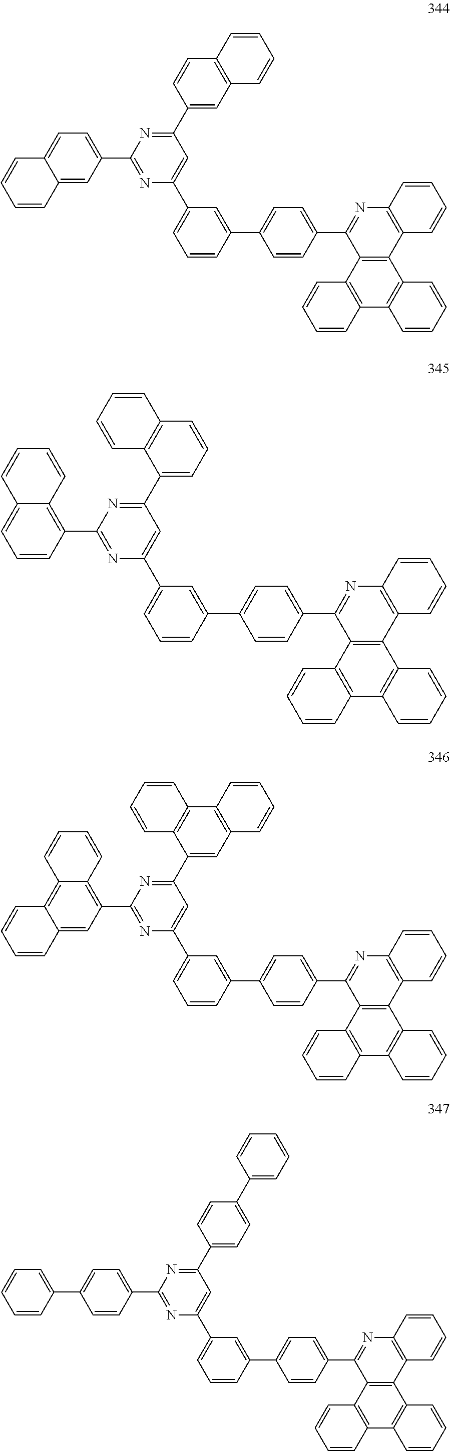 [SMILES: c1cc(-c2ccc(-c3nc4ccccc4c4c5ccccc5c5ccccc5c34)cc2)cc(-c2cc(-c3ccc4ccccc4c3)nc(-c3ccc4ccccc4c3)n2)c1 ; c1cc(-c2ccc(-c3nc4ccccc4c4c5ccccc5c5ccccc5c34)cc2)cc(-c2cc(-c3cccc4ccccc34)nc(-c3cccc4ccccc34)n2)c1 ; c1cc(-c2ccc(-c3nc4ccccc4c4c5ccccc5c5ccccc5c34)cc2)cc(-c2cc(-c3cc4ccccc4c4ccccc34)nc(-c3cc4ccccc4c4ccccc34)n2)c1 ; c1ccc(-c2ccc(-c3cc(-c4cccc(-c5ccc(-c6nc7ccccc7c7c8ccccc8c8ccccc8c67)cc5)c4)nc(-c4ccc(-c5ccccc5)cc4)n3)cc2)cc1]

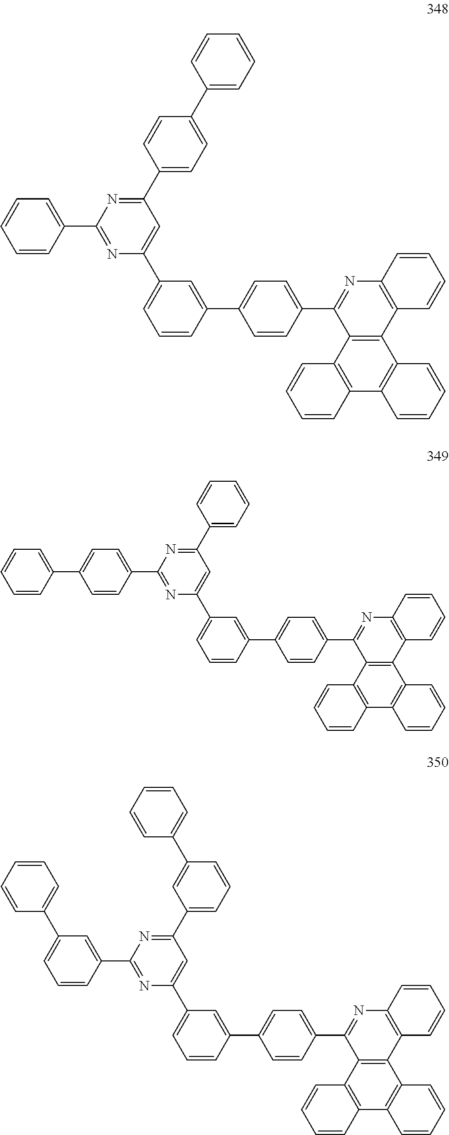 [SMILES: c1ccc(-c2ccc(-c3cc(-c4cccc(-c5ccc(-c6nc7ccccc7c7c8ccccc8c8ccccc8c67)cc5)c4)nc(-c4ccccc4)n3)cc2)cc1 ; c1ccc(-c2ccc(-c3nc(-c4ccccc4)cc(-c4cccc(-c5ccc(-c6nc7ccccc7c7c8ccccc8c8ccccc8c67)cc5)c4)n3)cc2)cc1 ; c1ccc(-c2cccc(-c3cc(-c4cccc(-c5ccc(-c6nc7ccccc7c7c8ccccc8c8ccccc8c67)cc5)c4)nc(-c4cccc(-c5ccccc5)c4)n3)c2)cc1]

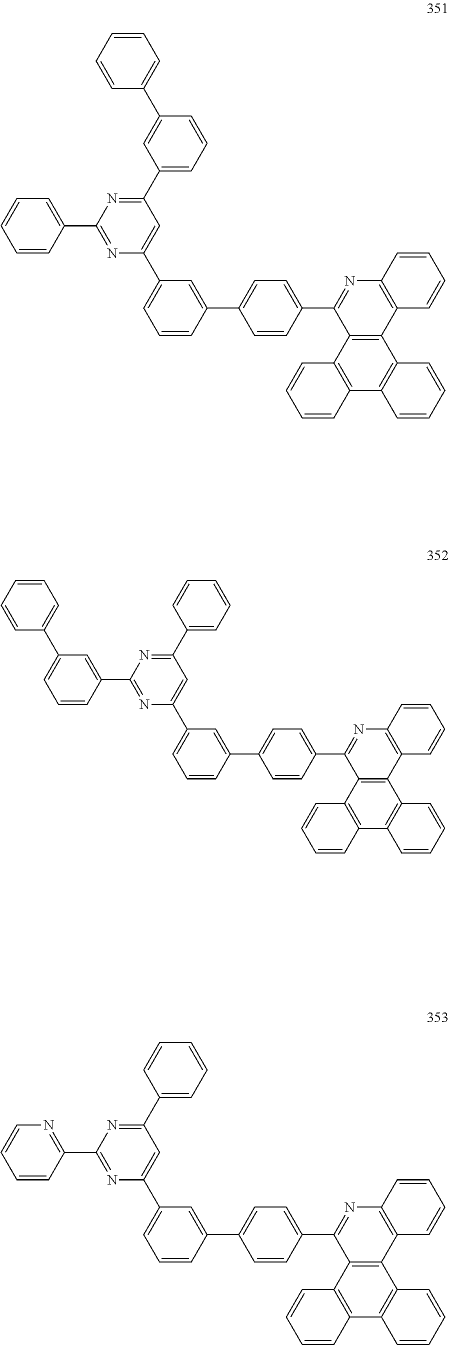 [SMILES: c1ccc(-c2cccc(-c3cc(-c4cccc(-c5ccc(-c6nc7ccccc7c7c8ccccc8c8ccccc8c67)cc5)c4)nc(-c4ccccc4)n3)c2)cc1 ; c1ccc(-c2cccc(-c3nc(-c4ccccc4)cc(-c4cccc(-c5ccc(-c6nc7ccccc7c7c8ccccc8c8ccccc8c67)cc5)c4)n3)c2)cc1 ; c1ccc(-c2cc(-c3cccc(-c4ccc(-c5nc6ccccc6c6c7ccccc7c7ccccc7c56)cc4)c3)nc(-c3ccccn3)n2)cc1]

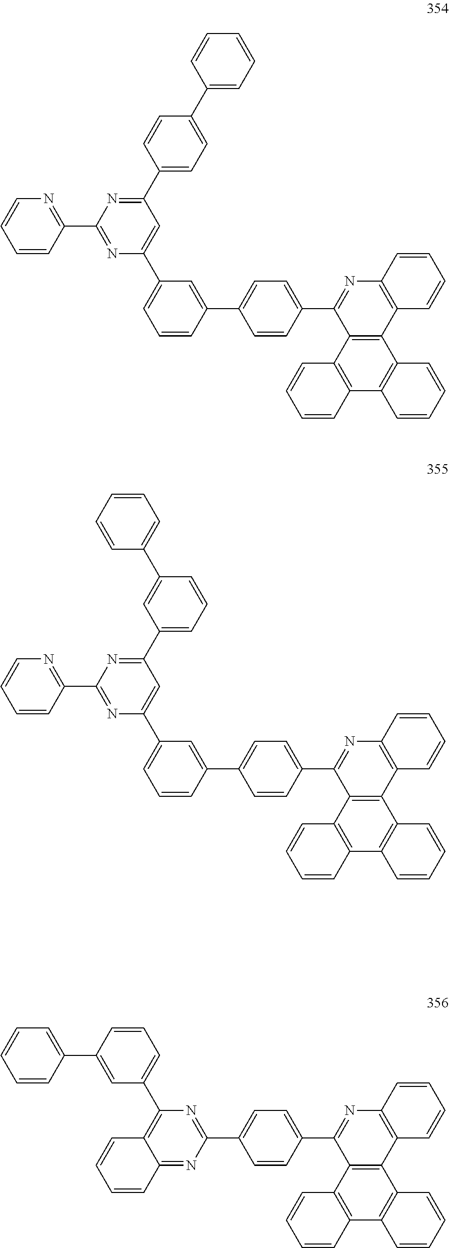 [SMILES: c1ccc(-c2ccc(-c3cc(-c4cccc(-c5ccc(-c6nc7ccccc7c7c8ccccc8c8ccccc8c67)cc5)c4)nc(-c4ccccn4)n3)cc2)cc1 ; c1ccc(-c2cccc(-c3cc(-c4cccc(-c5ccc(-c6nc7ccccc7c7c8ccccc8c8ccccc8c67)cc5)c4)nc(-c4ccccn4)n3)c2)cc1 ; c1ccc(-c2cccc(-c3nc(-c4ccc(-c5nc6ccccc6c6c7ccccc7c7ccccc7c56)cc4)nc4ccccc34)c2)cc1]

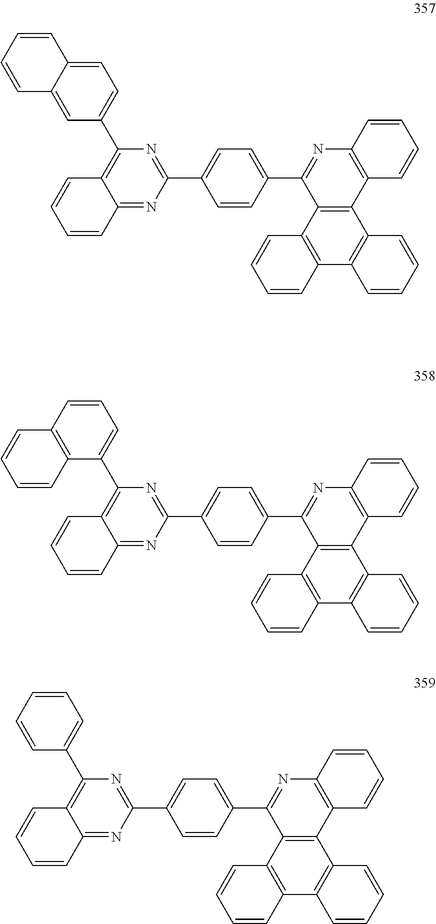 [SMILES: c1ccc2cc(-c3nc(-c4ccc(-c5nc6ccccc6c6c7ccccc7c7ccccc7c56)cc4)nc4ccccc34)ccc2c1 ; c1ccc2c(-c3nc(-c4ccc(-c5nc6ccccc6c6c7ccccc7c7ccccc7c56)cc4)nc4ccccc34)cccc2c1 ; c1ccc(-c2nc(-c3ccc(-c4nc5ccccc5c5c6ccccc6c6ccccc6c45)cc3)nc3ccccc23)cc1]

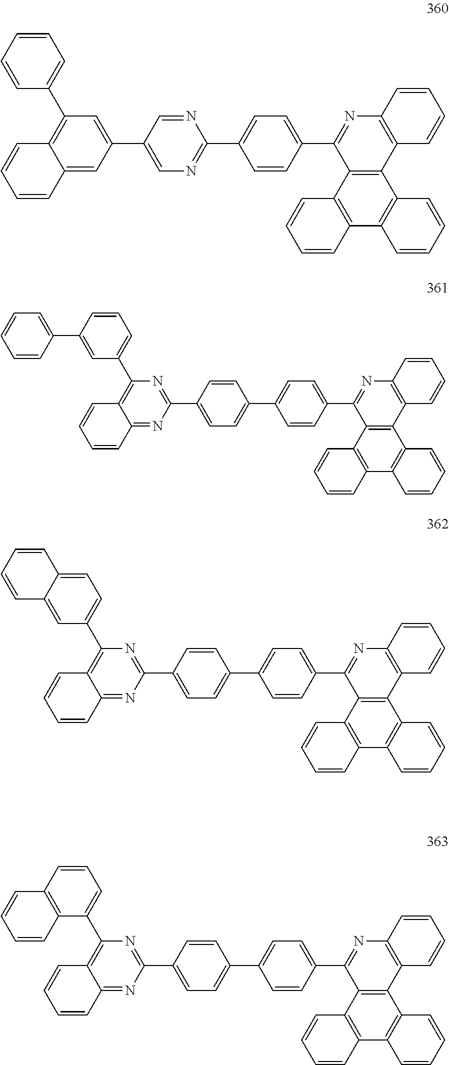 [SMILES: c1ccc(-c2cc(-c3cnc(-c4ccc(-c5nc6ccccc6c6c7ccccc7c7ccccc7c56)cc4)nc3)cc3ccccc23)cc1 ; c1ccc(-c2cccc(-c3nc(-c4ccc(-c5ccc(-c6nc7ccccc7c7c8ccccc8c8ccccc8c67)cc5)cc4)nc4ccccc34)c2)cc1 ; c1ccc2cc(-c3nc(-c4ccc(-c5ccc(-c6nc7ccccc7c7c8ccccc8c8ccccc8c67)cc5)cc4)nc4ccccc34)ccc2c1 ; c1ccc2c(-c3nc(-c4ccc(-c5ccc(-c6nc7ccccc7c7c8ccccc8c8ccccc8c67)cc5)cc4)nc4ccccc34)cccc2c1]

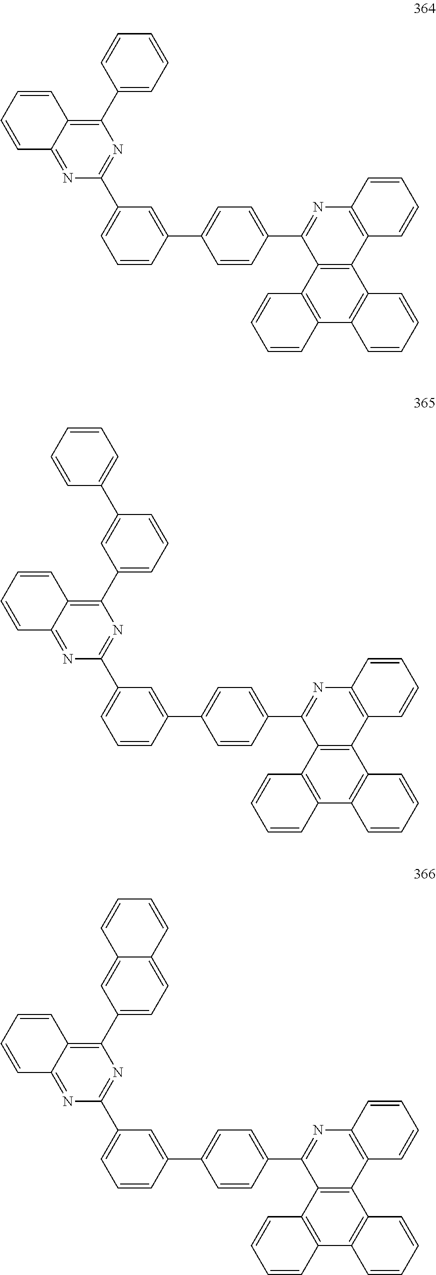 [SMILES: c1ccc(-c2nc(-c3cccc(-c4ccc(-c5nc6ccccc6c6c7ccccc7c7ccccc7c56)cc4)c3)nc3ccccc23)cc1 ; c1ccc(-c2cccc(-c3nc(-c4cccc(-c5ccc(-c6nc7ccccc7c7c8ccccc8c8ccccc8c67)cc5)c4)nc4ccccc34)c2)cc1 ; c1cc(-c2ccc(-c3nc4ccccc4c4c5ccccc5c5ccccc5c34)cc2)cc(-c2nc(-c3ccc4ccccc4c3)c3ccccc3n2)c1]

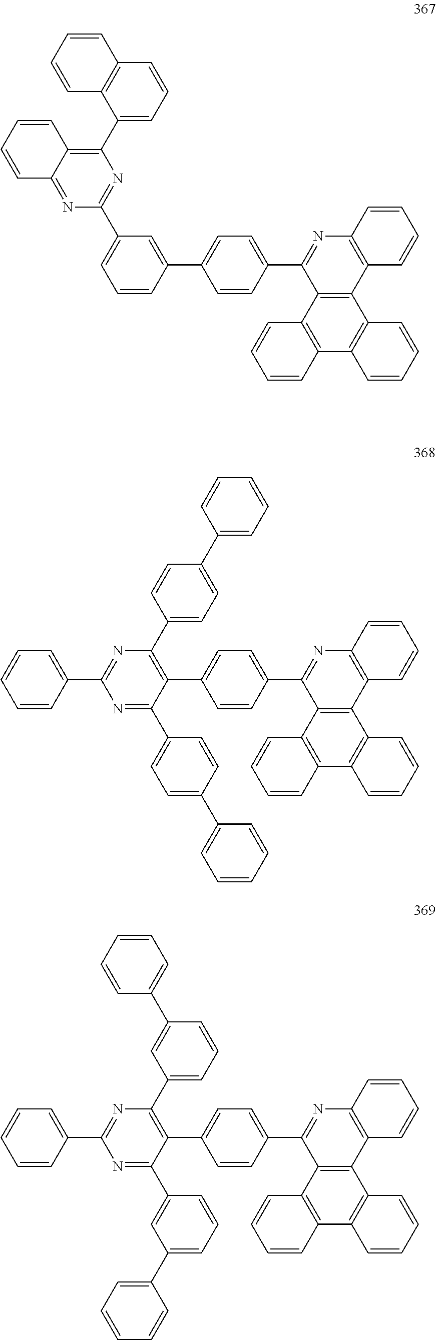 [SMILES: c1cc(-c2ccc(-c3nc4ccccc4c4c5ccccc5c5ccccc5c34)cc2)cc(-c2nc(-c3cccc4ccccc34)c3ccccc3n2)c1 ; c1ccc(-c2ccc(-c3nc(-c4ccccc4)nc(-c4ccc(-c5ccccc5)cc4)c3-c3ccc(-c4nc5ccccc5c5c6ccccc6c6ccccc6c45)cc3)cc2)cc1 ; c1ccc(-c2cccc(-c3nc(-c4ccccc4)nc(-c4cccc(-c5ccccc5)c4)c3-c3ccc(-c4nc5ccccc5c5c6ccccc6c6ccccc6c45)cc3)c2)cc1]

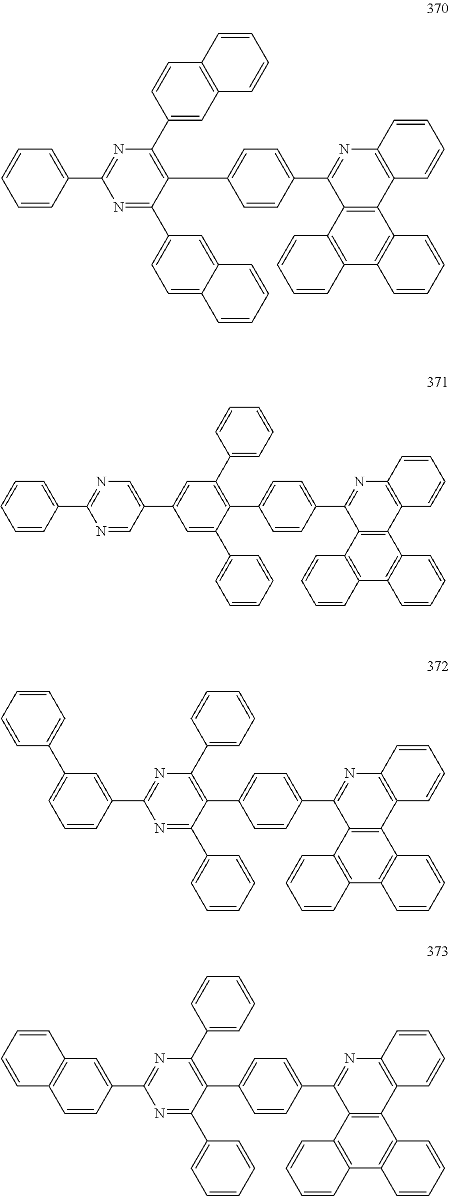 [SMILES: c1ccc(-c2nc(-c3ccc4ccccc4c3)c(-c3ccc(-c4nc5ccccc5c5c6ccccc6c6ccccc6c45)cc3)c(-c3ccc4ccccc4c3)n2)cc1 ; c1ccc(-c2ncc(-c3cc(-c4ccccc4)c(-c4ccc(-c5nc6ccccc6c6c7ccccc7c7ccccc7c56)cc4)c(-c4ccccc4)c3)cn2)cc1 ; c1ccc(-c2cccc(-c3nc(-c4ccccc4)c(-c4ccc(-c5nc6ccccc6c6c7ccccc7c7ccccc7c56)cc4)c(-c4ccccc4)n3)c2)cc1 ; c1ccc(-c2nc(-c3ccc4ccccc4c3)nc(-c3ccccc3)c2-c2ccc(-c3nc4ccccc4c4c5ccccc5c5ccccc5c34)cc2)cc1]

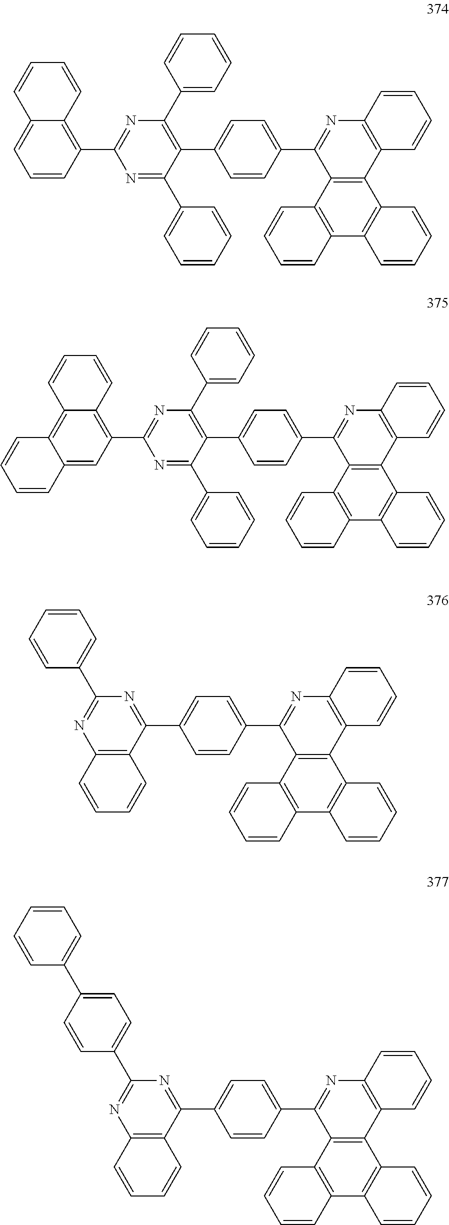 [SMILES: c1ccc(-c2nc(-c3cccc4ccccc34)nc(-c3ccccc3)c2-c2ccc(-c3nc4ccccc4c4c5ccccc5c5ccccc5c34)cc2)cc1 ; c1ccc(-c2nc(-c3cc4ccccc4c4ccccc34)nc(-c3ccccc3)c2-c2ccc(-c3nc4ccccc4c4c5ccccc5c5ccccc5c34)cc2)cc1 ; c1ccc(-c2nc(-c3ccc(-c4nc5ccccc5c5c6ccccc6c6ccccc6c45)cc3)c3ccccc3n2)cc1 ; c1ccc(-c2ccc(-c3nc(-c4ccc(-c5nc6ccccc6c6c7ccccc7c7ccccc7c56)cc4)c4ccccc4n3)cc2)cc1]

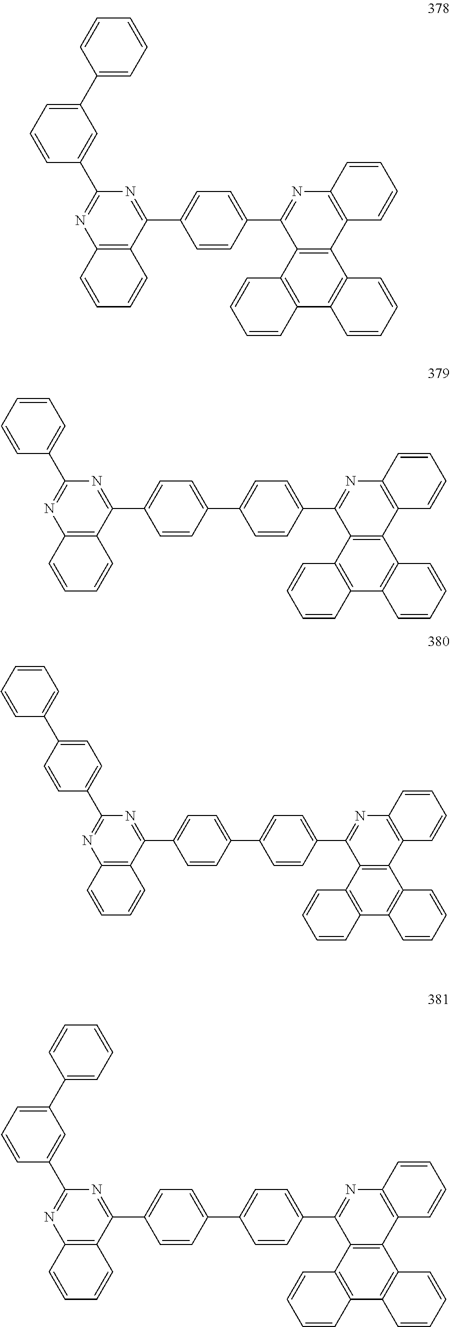 [SMILES: c1ccc(-c2cccc(-c3nc(-c4ccc(-c5nc6ccccc6c6c7ccccc7c7ccccc7c56)cc4)c4ccccc4n3)c2)cc1 ; c1ccc(-c2nc(-c3ccc(-c4ccc(-c5nc6ccccc6c6c7ccccc7c7ccccc7c56)cc4)cc3)c3ccccc3n2)cc1 ; c1ccc(-c2ccc(-c3nc(-c4ccc(-c5ccc(-c6nc7ccccc7c7c8ccccc8c8ccccc8c67)cc5)cc4)c4ccccc4n3)cc2)cc1 ; c1ccc(-c2cccc(-c3nc(-c4ccc(-c5ccc(-c6nc7ccccc7c7c8ccccc8c8ccccc8c67)cc5)cc4)c4ccccc4n3)c2)cc1]

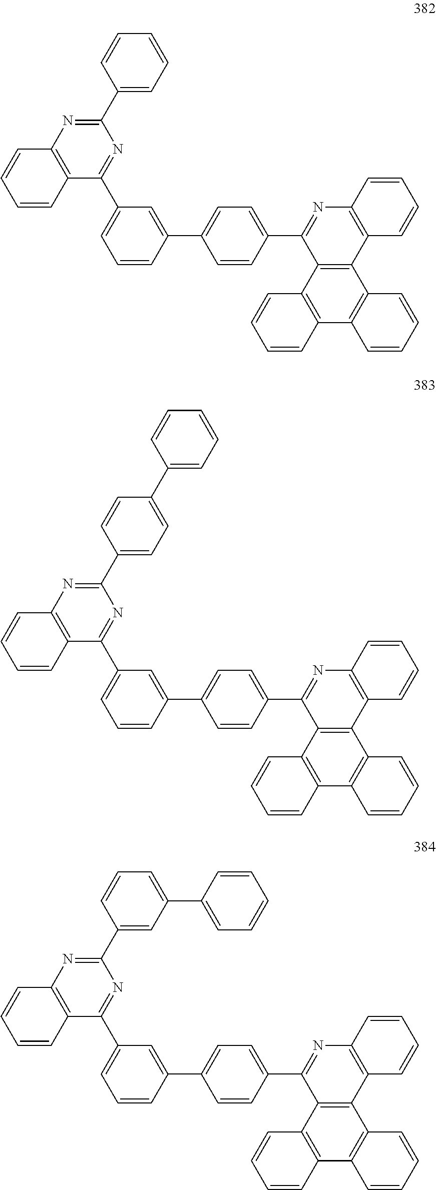 [SMILES: c1ccc(-c2nc(-c3cccc(-c4ccc(-c5nc6ccccc6c6c7ccccc7c7ccccc7c56)cc4)c3)c3ccccc3n2)cc1 ; c1ccc(-c2ccc(-c3nc(-c4cccc(-c5ccc(-c6nc7ccccc7c7c8ccccc8c8ccccc8c67)cc5)c4)c4ccccc4n3)cc2)cc1 ; c1ccc(-c2cccc(-c3nc(-c4cccc(-c5ccc(-c6nc7ccccc7c7c8ccccc8c8ccccc8c67)cc5)c4)c4ccccc4n3)c2)cc1]

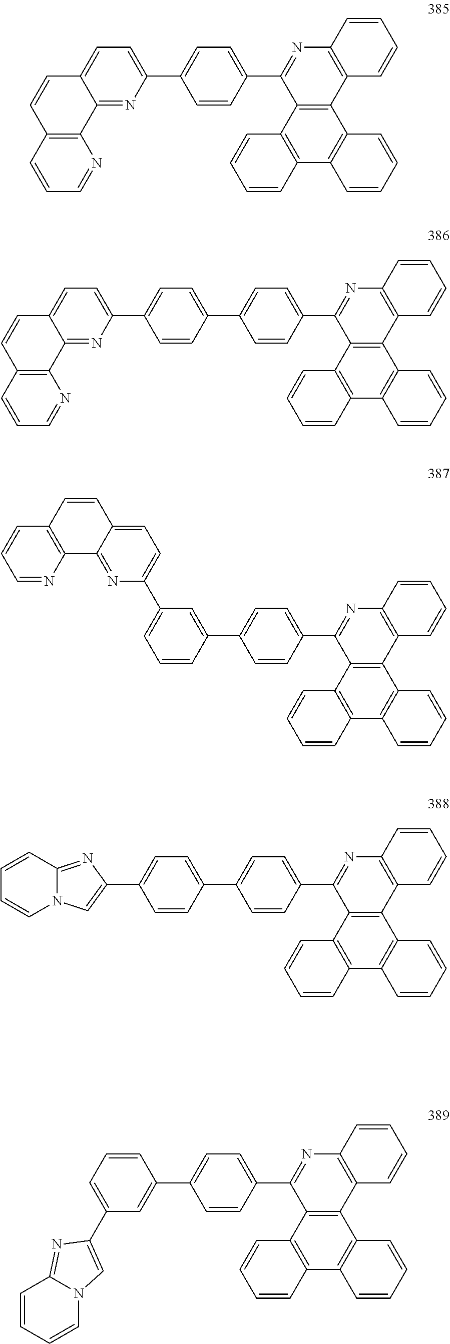 [SMILES: c1cnc2c(c1)ccc1ccc(-c3ccc(-c4nc5ccccc5c5c6ccccc6c6ccccc6c45)cc3)nc12 ; c1cnc2c(c1)ccc1ccc(-c3ccc(-c4ccc(-c5nc6ccccc6c6c7ccccc7c7ccccc7c56)cc4)cc3)nc12 ; c1cc(-c2ccc(-c3nc4ccccc4c4c5ccccc5c5ccccc5c34)cc2)cc(-c2ccc3ccc4cccnc4c3n2)c1 ; c1ccc2c(c1)nc(-c1ccc(-c3ccc(-c4cn5ccccc5n4)cc3)cc1)c1c3ccccc3c3ccccc3c21 ; c1cc(-c2ccc(-c3nc4ccccc4c4c5ccccc5c5ccccc5c34)cc2)cc(-c2cn3ccccc3n2)c1]

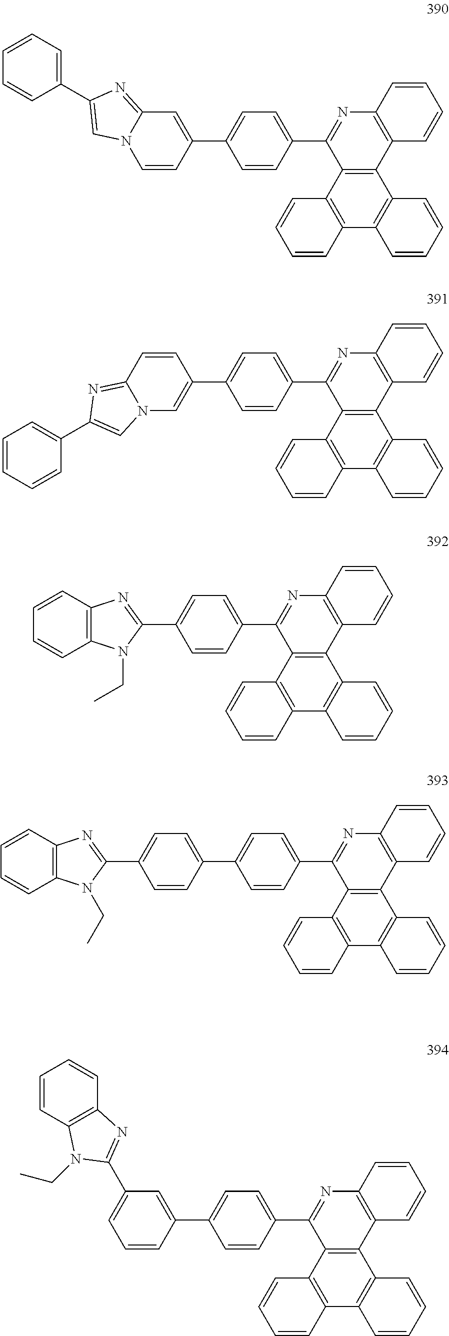 [SMILES: c1ccc(-c2cn3ccc(-c4ccc(-c5nc6ccccc6c6c7ccccc7c7ccccc7c56)cc4)cc3n2)cc1 ; c1ccc(-c2cn3cc(-c4ccc(-c5nc6ccccc6c6c7ccccc7c7ccccc7c56)cc4)ccc3n2)cc1 ; CCn1c(-c2ccc(-c3nc4ccccc4c4c5ccccc5c5ccccc5c34)cc2)nc2ccccc21 ; CCn1c(-c2ccc(-c3ccc(-c4nc5ccccc5c5c6ccccc6c6ccccc6c45)cc3)cc2)nc2ccccc21 ; CCn1c(-c2cccc(-c3ccc(-c4nc5ccccc5c5c6ccccc6c6ccccc6c45)cc3)c2)nc2ccccc21]

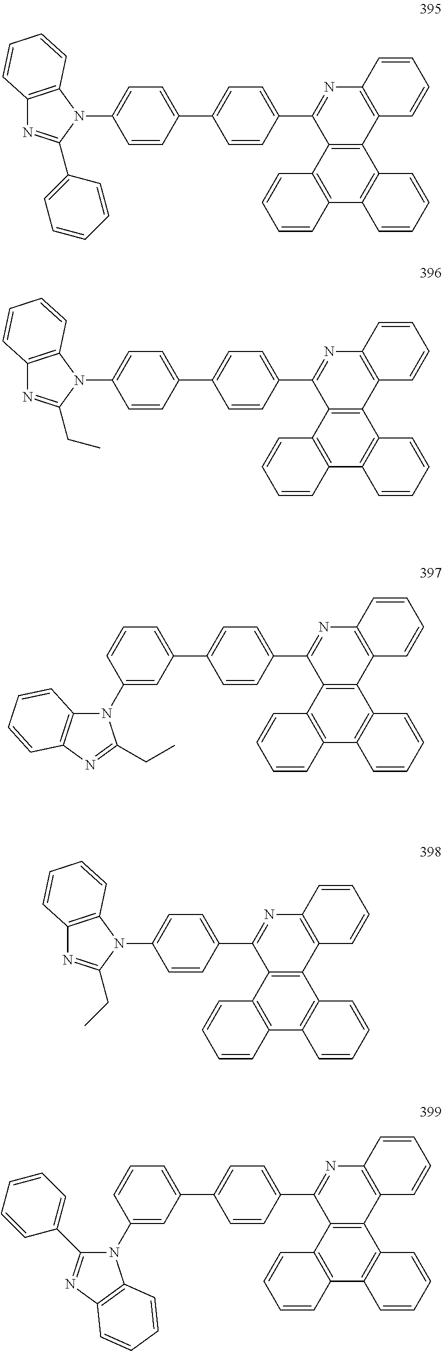 [SMILES: c1ccc(-c2nc3ccccc3n2-c2ccc(-c3ccc(-c4nc5ccccc5c5c6ccccc6c6ccccc6c45)cc3)cc2)cc1 ; CCc1nc2ccccc2n1-c1ccc(-c2ccc(-c3nc4ccccc4c4c5ccccc5c5ccccc5c34)cc2)cc1 ; CCc1nc2ccccc2n1-c1cccc(-c2ccc(-c3nc4ccccc4c4c5ccccc5c5ccccc5c34)cc2)c1 ; CCc1nc2ccccc2n1-c1ccc(-c2nc3ccccc3c3c4ccccc4c4ccccc4c23)cc1 ; c1ccc(-c2nc3ccccc3n2-c2cccc(-c3ccc(-c4nc5ccccc5c5c6ccccc6c6ccccc6c45)cc3)c2)cc1]

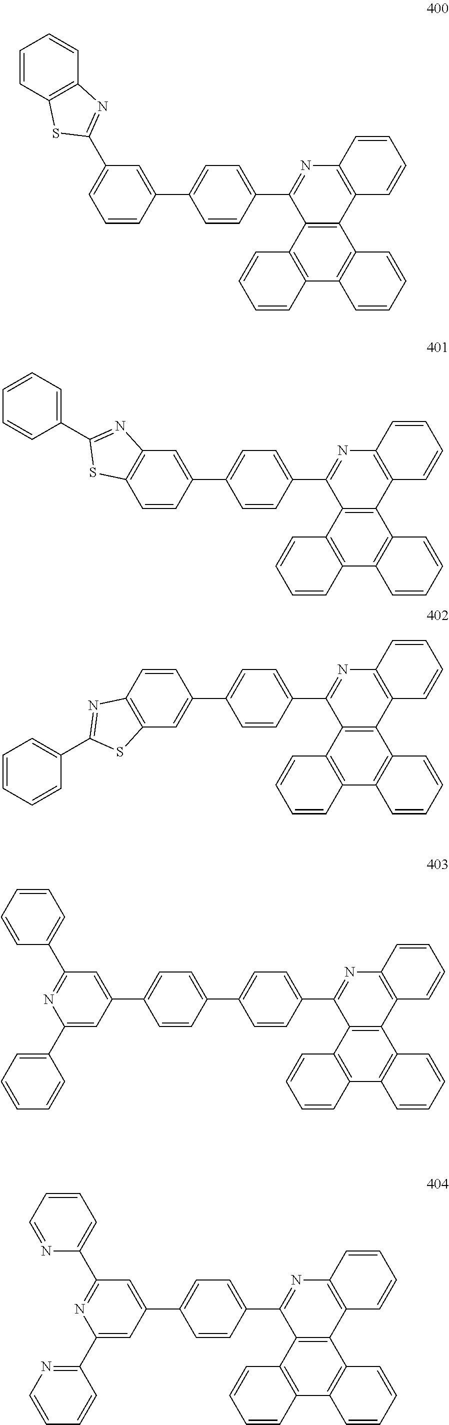

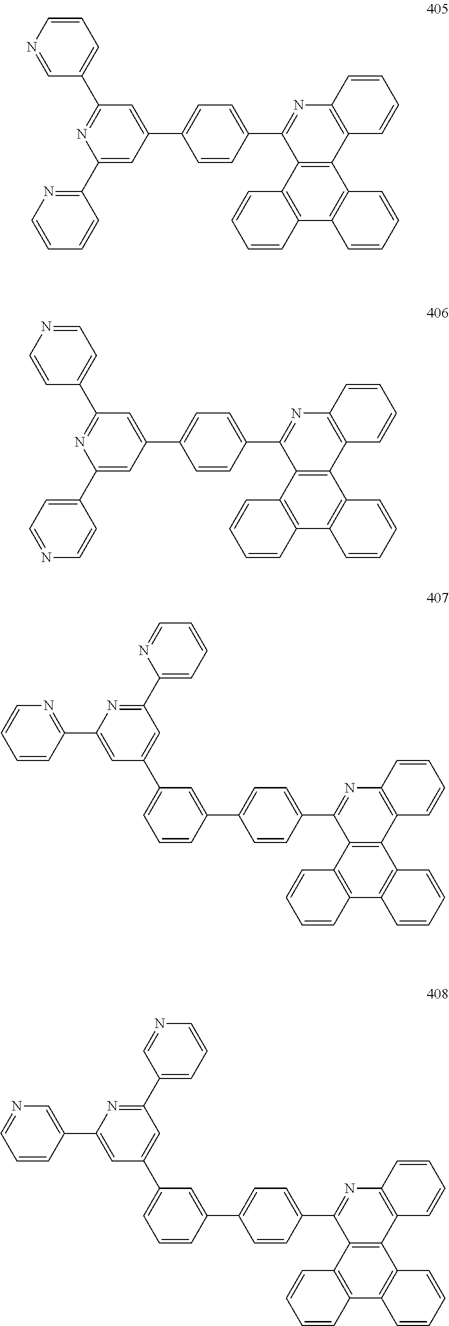 [SMILES: c1ccc(-c2cc(-c3ccc(-c4nc5ccccc5c5c6ccccc6c6ccccc6c45)cc3)cc(-c3cccnc3)n2)nc1 ; c1ccc2c(c1)nc(-c1ccc(-c3cc(-c4ccncc4)nc(-c4ccncc4)c3)cc1)c1c3ccccc3c3ccccc3c21 ; c1ccc(-c2cc(-c3cccc(-c4ccc(-c5nc6ccccc6c6c7ccccc7c7ccccc7c56)cc4)c3)cc(-c3ccccn3)n2)nc1 ; c1cc(-c2ccc(-c3nc4ccccc4c4c5ccccc5c5ccccc5c34)cc2)cc(-c2cc(-c3cccnc3)nc(-c3cccnc3)c2)c1]

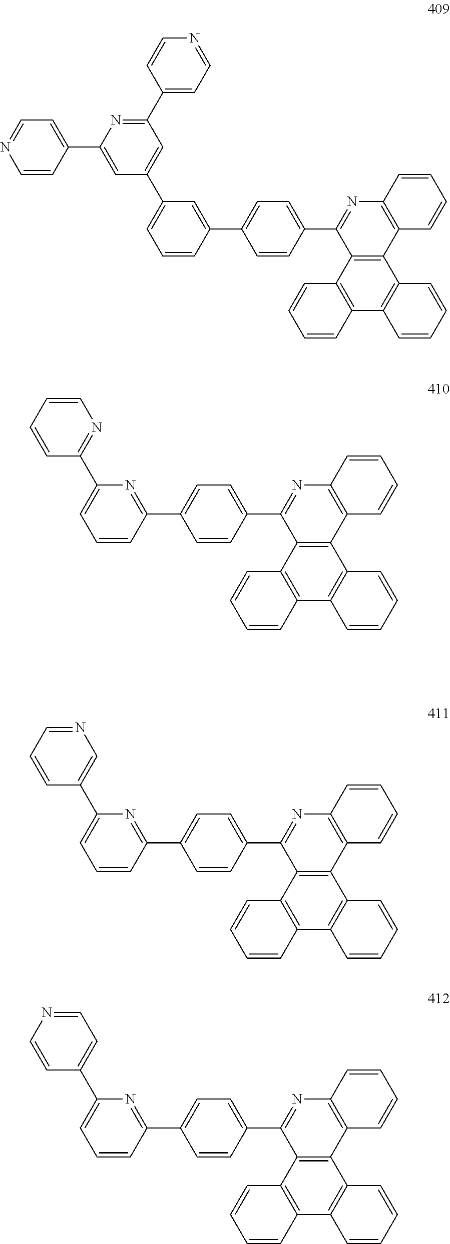 [SMILES: c1cc(-c2ccc(-c3nc4ccccc4c4c5ccccc5c5ccccc5c34)cc2)cc(-c2cc(-c3ccncc3)nc(-c3ccncc3)c2)c1 ; c1ccc(-c2cccc(-c3ccc(-c4nc5ccccc5c5c6ccccc6c6ccccc6c45)cc3)n2)nc1 ; c1cncc(-c2cccc(-c3ccc(-c4nc5ccccc5c5c6ccccc6c6ccccc6c45)cc3)n2)c1 ; c1cc(-c2ccncc2)nc(-c2ccc(-c3nc4ccccc4c4c5ccccc5c5ccccc5c34)cc2)c1]

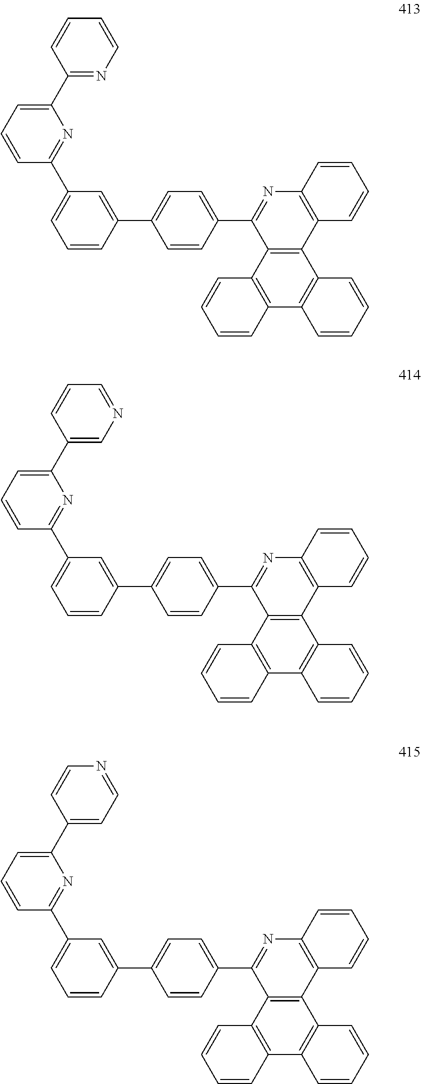 [SMILES: c1ccc(-c2cccc(-c3cccc(-c4ccc(-c5nc6ccccc6c6c7ccccc7c7ccccc7c56)cc4)c3)n2)nc1 ; c1cncc(-c2cccc(-c3cccc(-c4ccc(-c5nc6ccccc6c6c7ccccc7c7ccccc7c56)cc4)c3)n2)c1 ; c1cc(-c2ccc(-c3nc4ccccc4c4c5ccccc5c5ccccc5c34)cc2)cc(-c2cccc(-c3ccncc3)n2)c1]

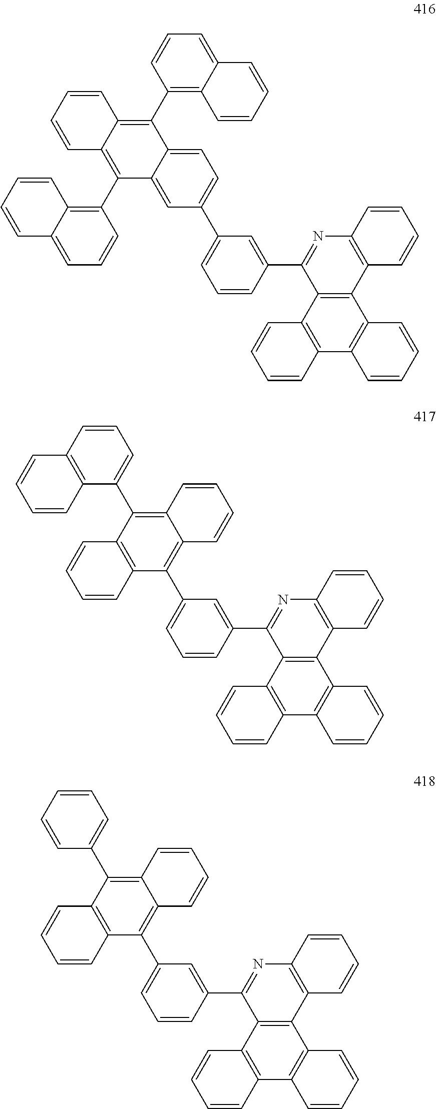 [SMILES: c1cc(-c2ccc3c(-c4cccc5ccccc45)c4ccccc4c(-c4cccc5ccccc45)c3c2)cc(-c2nc3ccccc3c3c4ccccc4c4ccccc4c23)c1 ; c1cc(-c2c3ccccc3c(-c3cccc4ccccc34)c3ccccc23)cc(-c2nc3ccccc3c3c4ccccc4c4ccccc4c23)c1 ; c1ccc(-c2c3ccccc3c(-c3cccc(-c4nc5ccccc5c5c6ccccc6c6ccccc6c45)c3)c3ccccc23)cc1]

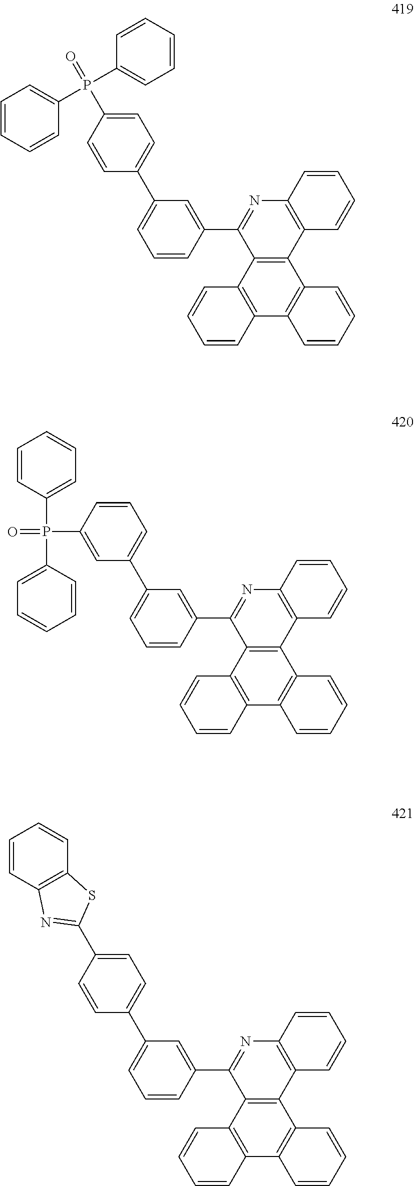 [SMILES: O=P(c1ccccc1)(c1ccccc1)c1ccc(-c2cccc(-c3nc4ccccc4c4c5ccccc5c5ccccc5c34)c2)cc1 ; O=P(c1ccccc1)(c1ccccc1)c1cccc(-c2cccc(-c3nc4ccccc4c4c5ccccc5c5ccccc5c34)c2)c1 ; c1cc(-c2ccc(-c3nc4ccccc4s3)cc2)cc(-c2nc3ccccc3c3c4ccccc4c4ccccc4c23)c1]

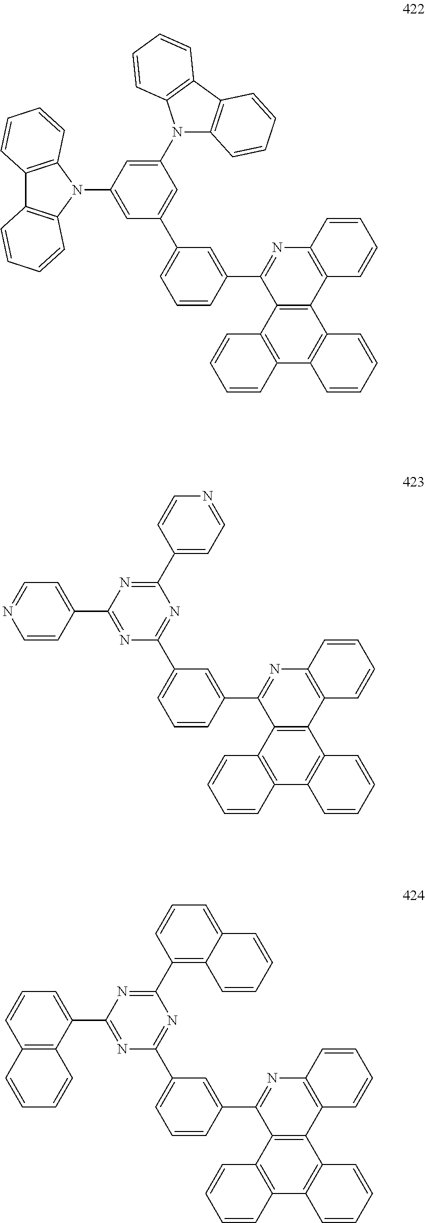 [SMILES: c1cc(-c2cc(-n3c4ccccc4c4ccccc43)cc(-n3c4ccccc4c4ccccc43)c2)cc(-c2nc3ccccc3c3c4ccccc4c4ccccc4c23)c1 ; c1cc(-c2nc(-c3ccncc3)nc(-c3ccncc3)n2)cc(-c2nc3ccccc3c3c4ccccc4c4ccccc4c23)c1 ; c1cc(-c2nc(-c3cccc4ccccc34)nc(-c3cccc4ccccc34)n2)cc(-c2nc3ccccc3c3c4ccccc4c4ccccc4c23)c1]

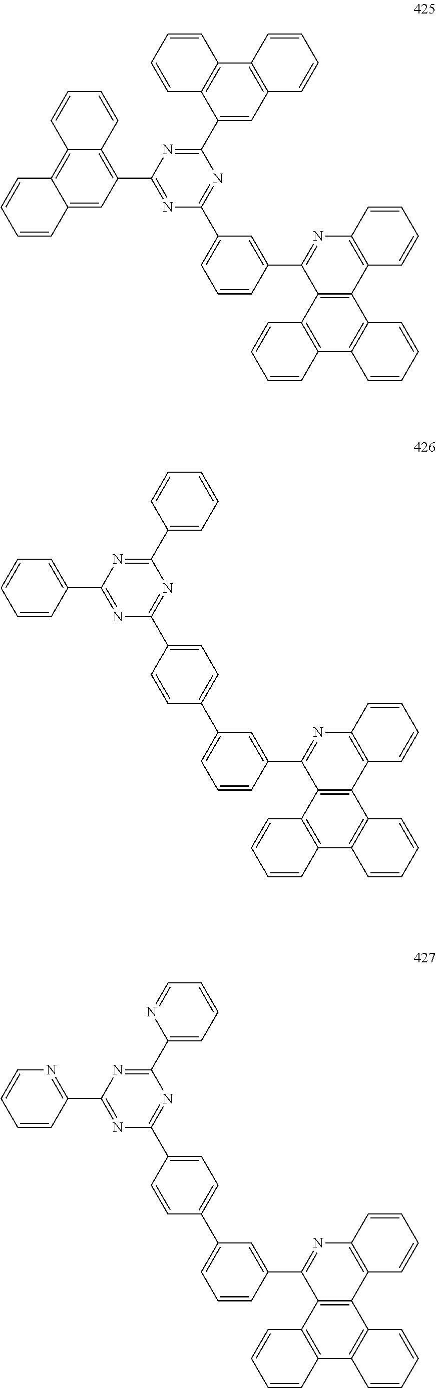 [SMILES: c1cc(-c2nc(-c3cc4ccccc4c4ccccc34)nc(-c3cc4ccccc4c4ccccc34)n2)cc(-c2nc3ccccc3c3c4ccccc4c4ccccc4c23)c1 ; c1ccc(-c2nc(-c3ccccc3)nc(-c3ccc(-c4cccc(-c5nc6ccccc6c6c7ccccc7c7ccccc7c56)c4)cc3)n2)cc1 ; c1ccc(-c2nc(-c3ccc(-c4cccc(-c5nc6ccccc6c6c7ccccc7c7ccccc7c56)c4)cc3)nc(-c3ccccn3)n2)nc1]

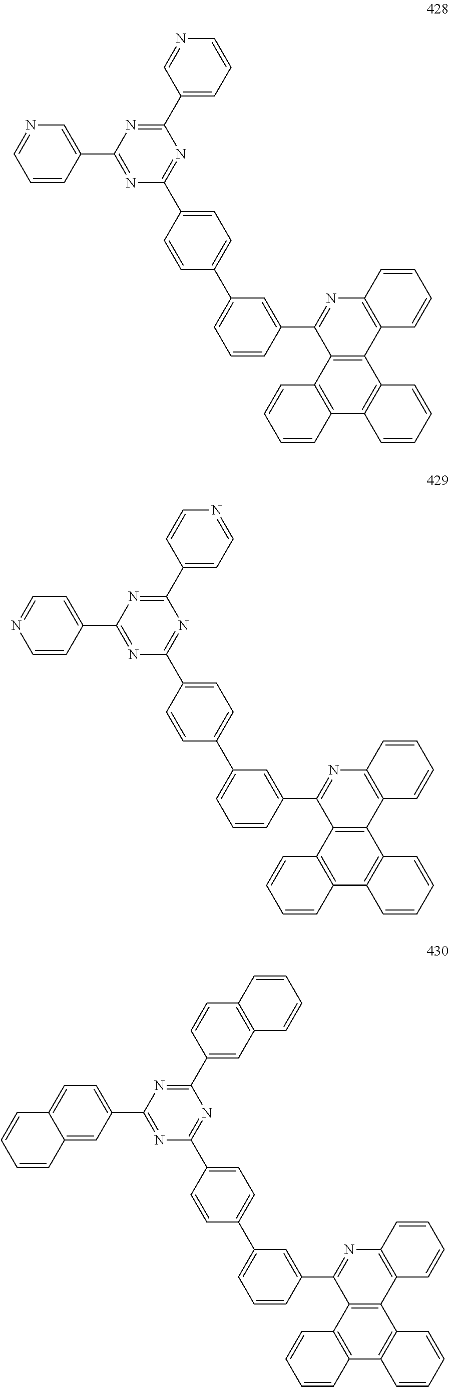 [SMILES: c1cncc(-c2nc(-c3ccc(-c4cccc(-c5nc6ccccc6c6c7ccccc7c7ccccc7c56)c4)cc3)nc(-c3cccnc3)n2)c1 ; c1cc(-c2ccc(-c3nc(-c4ccncc4)nc(-c4ccncc4)n3)cc2)cc(-c2nc3ccccc3c3c4ccccc4c4ccccc4c23)c1 ; c1cc(-c2ccc(-c3nc(-c4ccc5ccccc5c4)nc(-c4ccc5ccccc5c4)n3)cc2)cc(-c2nc3ccccc3c3c4ccccc4c4ccccc4c23)c1]

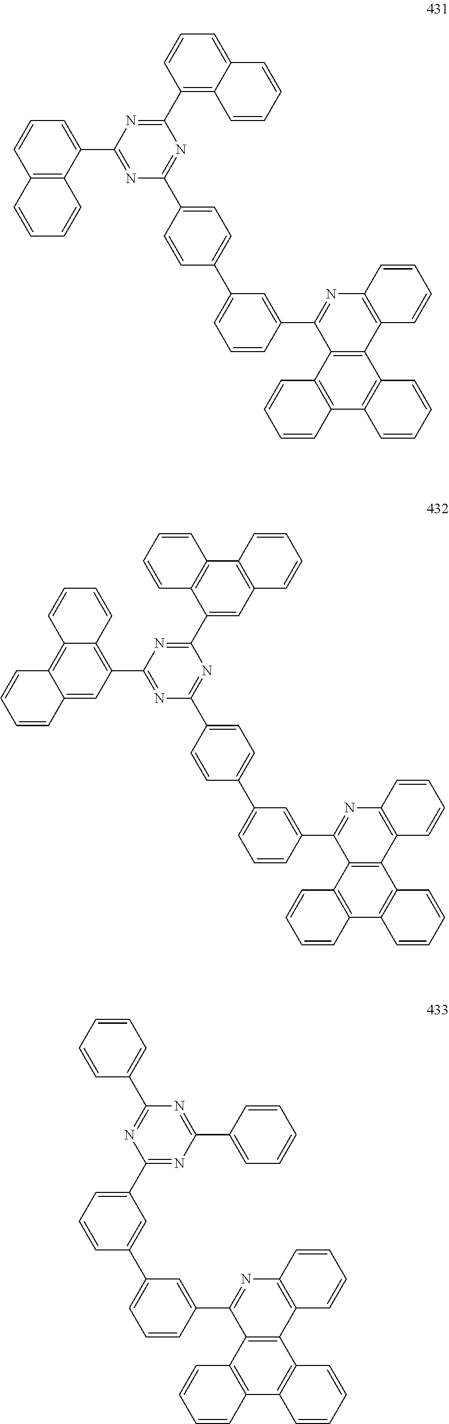 [SMILES: c1cc(-c2ccc(-c3nc(-c4cccc5ccccc45)nc(-c4cccc5ccccc45)n3)cc2)cc(-c2nc3ccccc3c3c4ccccc4c4ccccc4c23)c1 ; c1cc(-c2ccc(-c3nc(-c4cc5ccccc5c5ccccc45)nc(-c4cc5ccccc5c5ccccc45)n3)cc2)cc(-c2nc3ccccc3c3c4ccccc4c4ccccc4c23)c1 ; c1ccc(-c2nc(-c3ccccc3)nc(-c3cccc(-c4cccc(-c5nc6ccccc6c6c7ccccc7c7ccccc7c56)c4)c3)n2)cc1]

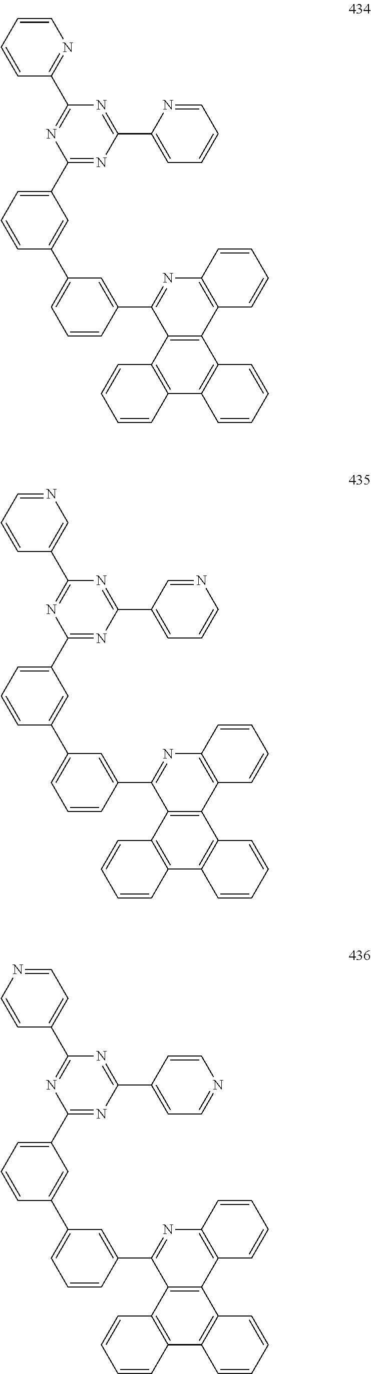 [SMILES: c1ccc(-c2nc(-c3cccc(-c4cccc(-c5nc6ccccc6c6c7ccccc7c7ccccc7c56)c4)c3)nc(-c3ccccn3)n2)nc1 ; c1cncc(-c2nc(-c3cccnc3)nc(-c3cccc(-c4cccc(-c5nc6ccccc6c6c7ccccc7c7ccccc7c56)c4)c3)n2)c1 ; c1cc(-c2cccc(-c3nc4ccccc4c4c5ccccc5c5ccccc5c34)c2)cc(-c2nc(-c3ccncc3)nc(-c3ccncc3)n2)c1]

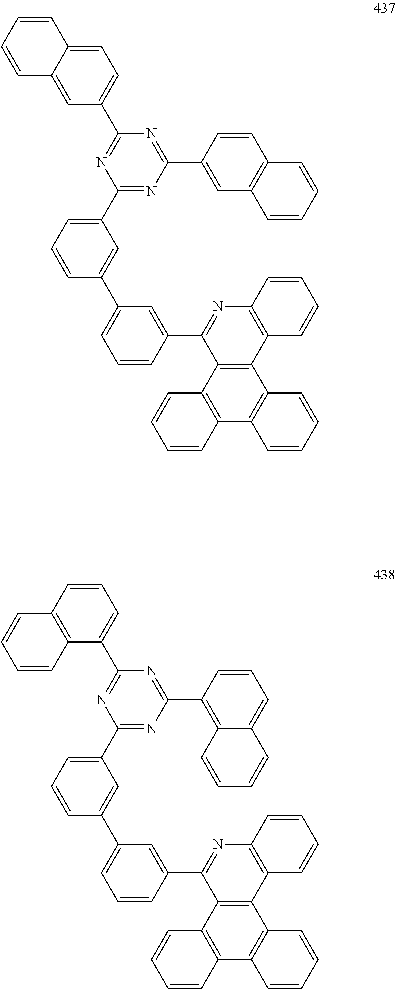 [SMILES: c1cc(-c2cccc(-c3nc4ccccc4c4c5ccccc5c5ccccc5c34)c2)cc(-c2nc(-c3ccc4ccccc4c3)nc(-c3ccc4ccccc4c3)n2)c1 ; c1cc(-c2cccc(-c3nc4ccccc4c4c5ccccc5c5ccccc5c34)c2)cc(-c2nc(-c3cccc4ccccc34)nc(-c3cccc4ccccc34)n2)c1]

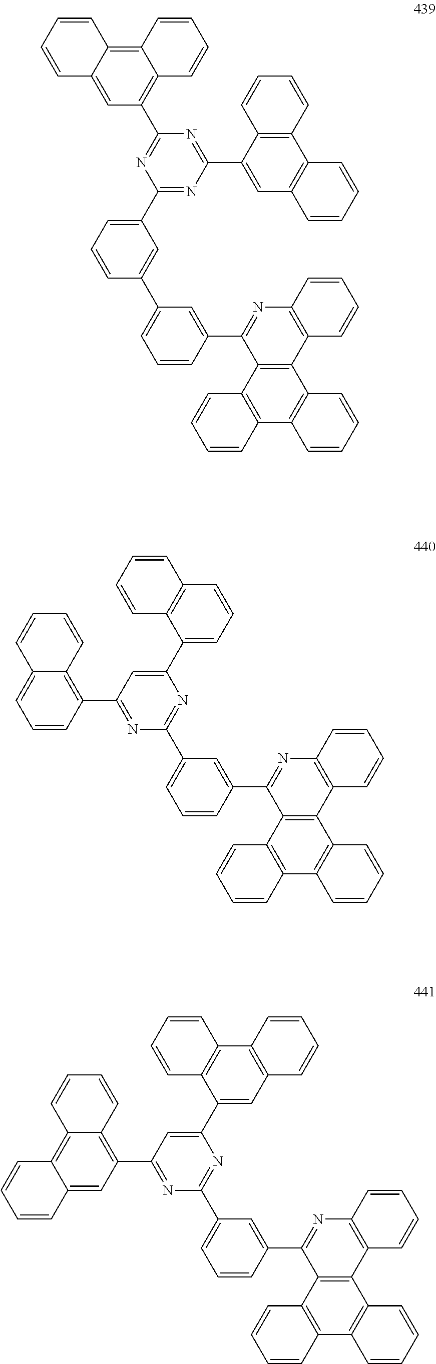 [SMILES: c1cc(-c2cccc(-c3nc4ccccc4c4c5ccccc5c5ccccc5c34)c2)cc(-c2nc(-c3cc4ccccc4c4ccccc34)nc(-c3cc4ccccc4c4ccccc34)n2)c1 ; c1cc(-c2nc(-c3cccc4ccccc34)cc(-c3cccc4ccccc34)n2)cc(-c2nc3ccccc3c3c4ccccc4c4ccccc4c23)c1 ; c1cc(-c2nc(-c3cc4ccccc4c4ccccc34)cc(-c3cc4ccccc4c4ccccc34)n2)cc(-c2nc3ccccc3c3c4ccccc4c4ccccc4c23)c1]

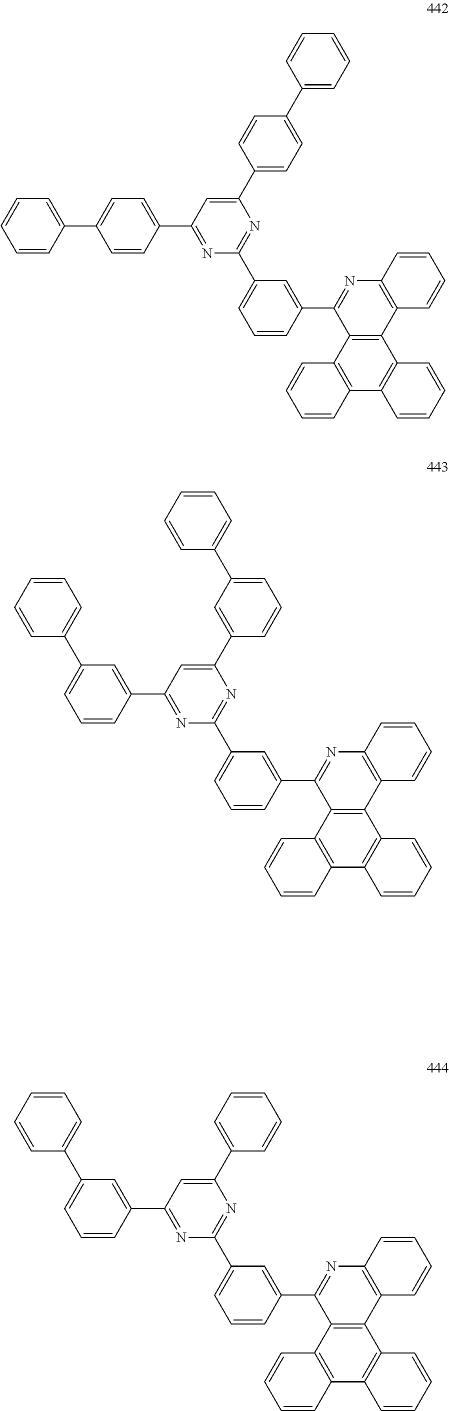 [SMILES: c1ccc(-c2ccc(-c3cc(-c4ccc(-c5ccccc5)cc4)nc(-c4cccc(-c5nc6ccccc6c6c7ccccc7c7ccccc7c56)c4)n3)cc2)cc1 ; c1ccc(-c2cccc(-c3cc(-c4cccc(-c5ccccc5)c4)nc(-c4cccc(-c5nc6ccccc6c6c7ccccc7c7ccccc7c56)c4)n3)c2)cc1 ; c1ccc(-c2cccc(-c3cc(-c4ccccc4)nc(-c4cccc(-c5nc6ccccc6c6c7ccccc7c7ccccc7c56)c4)n3)c2)cc1]

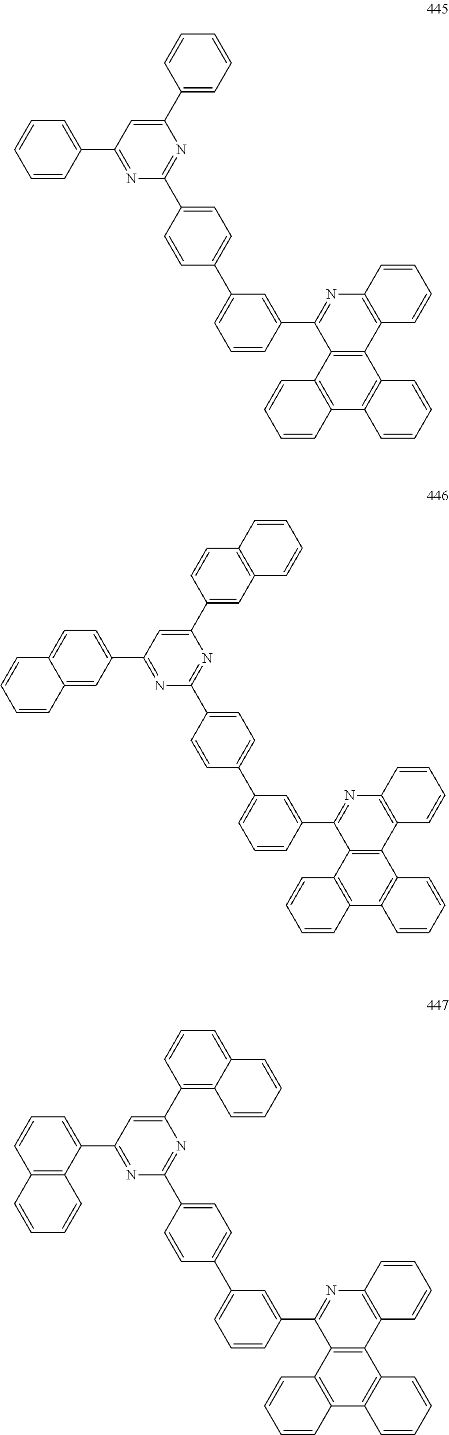 [SMILES: c1ccc(-c2cc(-c3ccccc3)nc(-c3ccc(-c4cccc(-c5nc6ccccc6c6c7ccccc7c7ccccc7c56)c4)cc3)n2)cc1 ; c1cc(-c2ccc(-c3nc(-c4ccc5ccccc5c4)cc(-c4ccc5ccccc5c4)n3)cc2)cc(-c2nc3ccccc3c3c4ccccc4c4ccccc4c23)c1 ; c1cc(-c2ccc(-c3nc(-c4cccc5ccccc45)cc(-c4cccc5ccccc45)n3)cc2)cc(-c2nc3ccccc3c3c4ccccc4c4ccccc4c23)c1]

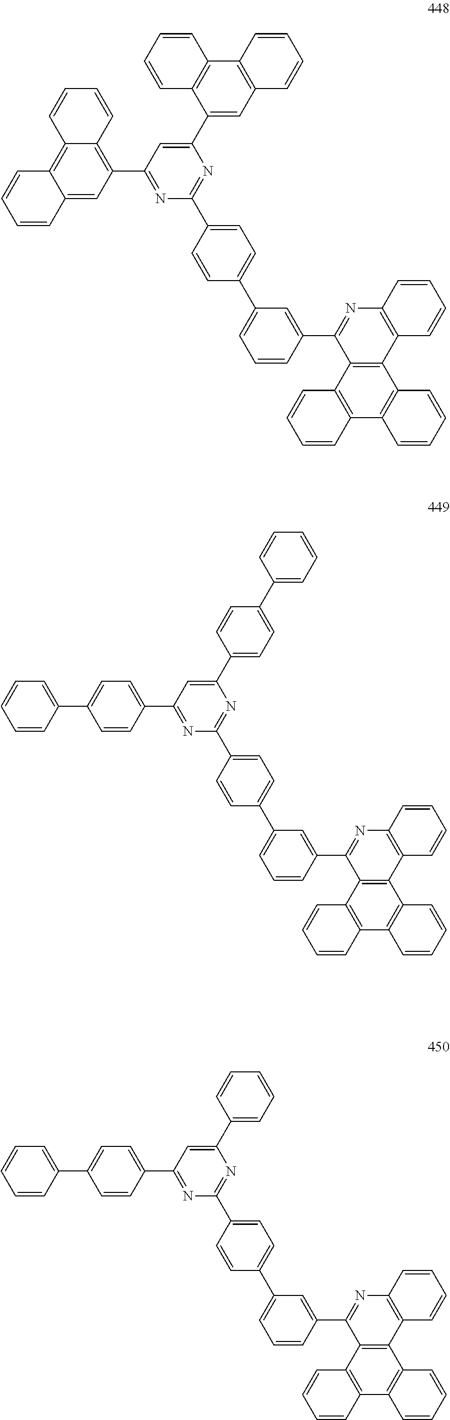 [SMILES: c1cc(-c2ccc(-c3nc(-c4cc5ccccc5c5ccccc45)cc(-c4cc5ccccc5c5ccccc45)n3)cc2)cc(-c2nc3ccccc3c3c4ccccc4c4ccccc4c23)c1 ; c1ccc(-c2ccc(-c3cc(-c4ccc(-c5ccccc5)cc4)nc(-c4ccc(-c5cccc(-c6nc7ccccc7c7c8ccccc8c8ccccc8c67)c5)cc4)n3)cc2)cc1 ; c1ccc(-c2ccc(-c3cc(-c4ccccc4)nc(-c4ccc(-c5cccc(-c6nc7ccccc7c7c8ccccc8c8ccccc8c67)c5)cc4)n3)cc2)cc1]

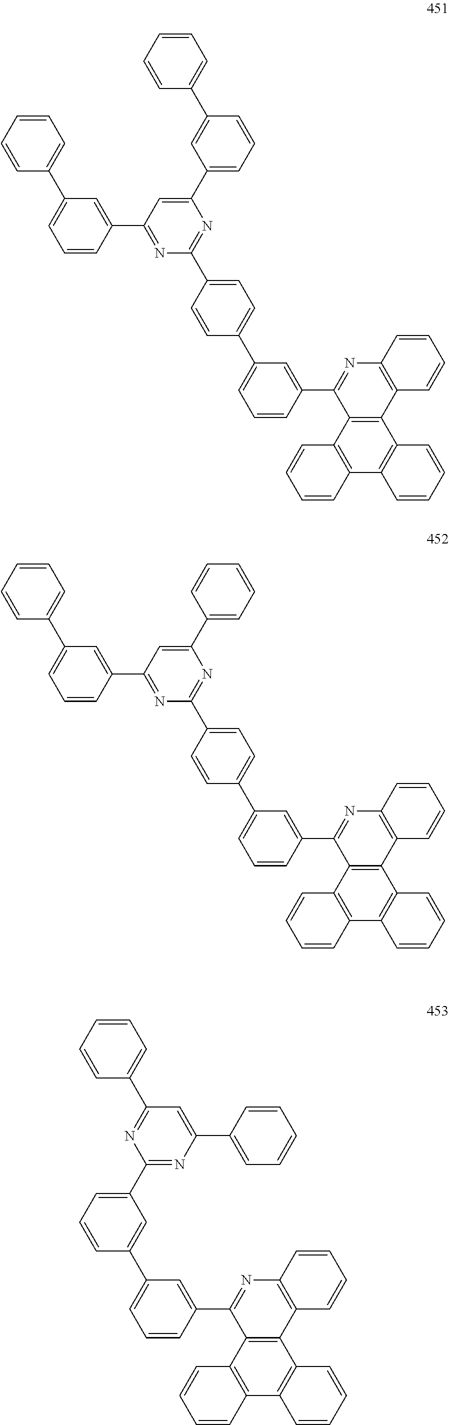 [SMILES: c1ccc(-c2cccc(-c3cc(-c4cccc(-c5ccccc5)c4)nc(-c4ccc(-c5cccc(-c6nc7ccccc7c7c8ccccc8c8ccccc8c67)c5)cc4)n3)c2)cc1 ; c1ccc(-c2cccc(-c3cc(-c4ccccc4)nc(-c4ccc(-c5cccc(-c6nc7ccccc7c7c8ccccc8c8ccccc8c67)c5)cc4)n3)c2)cc1 ; c1ccc(-c2cc(-c3ccccc3)nc(-c3cccc(-c4cccc(-c5nc6ccccc6c6c7ccccc7c7ccccc7c56)c4)c3)n2)cc1]

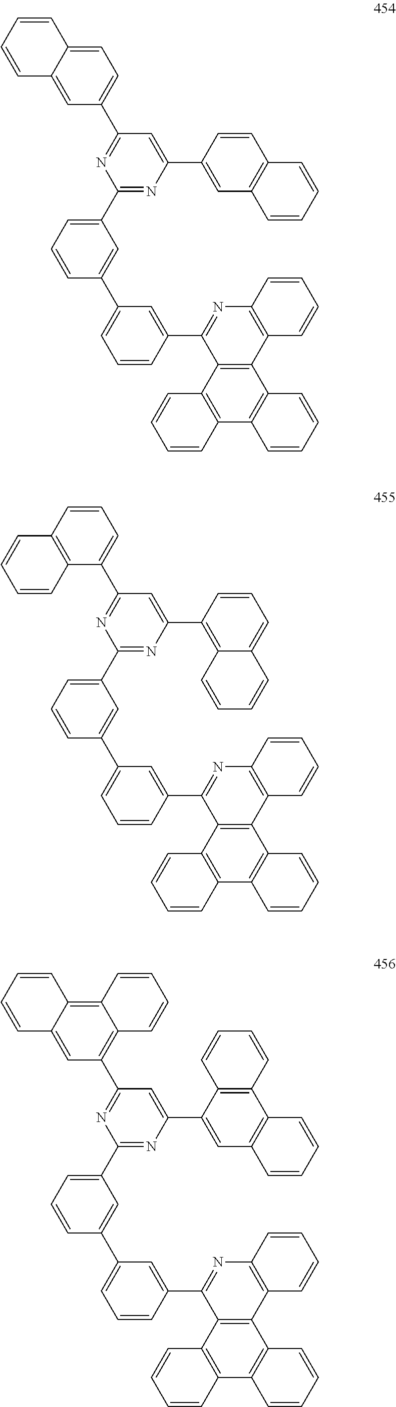 [SMILES: c1cc(-c2cccc(-c3nc4ccccc4c4c5ccccc5c5ccccc5c34)c2)cc(-c2nc(-c3ccc4ccccc4c3)cc(-c3ccc4ccccc4c3)n2)c1 ; c1cc(-c2cccc(-c3nc4ccccc4c4c5ccccc5c5ccccc5c34)c2)cc(-c2nc(-c3cccc4ccccc34)cc(-c3cccc4ccccc34)n2)c1 ; c1cc(-c2cccc(-c3nc4ccccc4c4c5ccccc5c5ccccc5c34)c2)cc(-c2nc(-c3cc4ccccc4c4ccccc34)cc(-c3cc4ccccc4c4ccccc34)n2)c1]

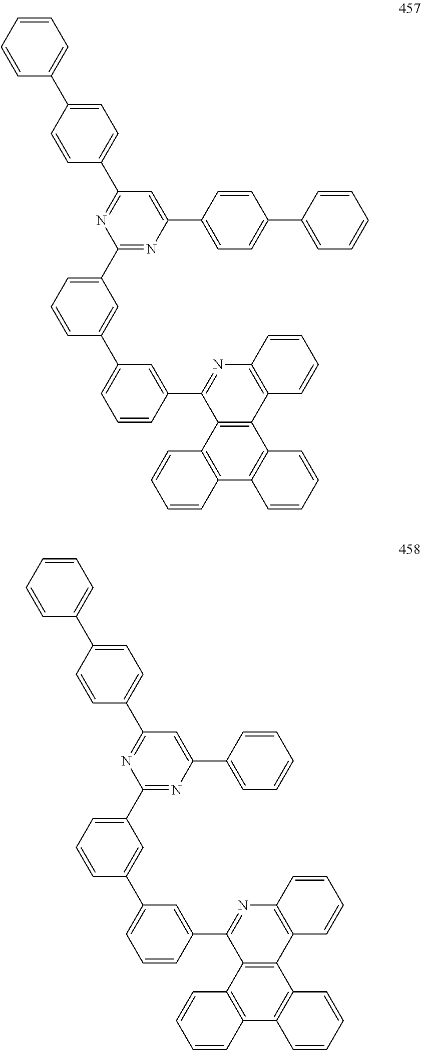 [SMILES: c1ccc(-c2ccc(-c3cc(-c4ccc(-c5ccccc5)cc4)nc(-c4cccc(-c5cccc(-c6nc7ccccc7c7c8ccccc8c8ccccc8c67)c5)c4)n3)cc2)cc1 ; c1ccc(-c2ccc(-c3cc(-c4ccccc4)nc(-c4cccc(-c5cccc(-c6nc7ccccc7c7c8ccccc8c8ccccc8c67)c5)c4)n3)cc2)cc1]

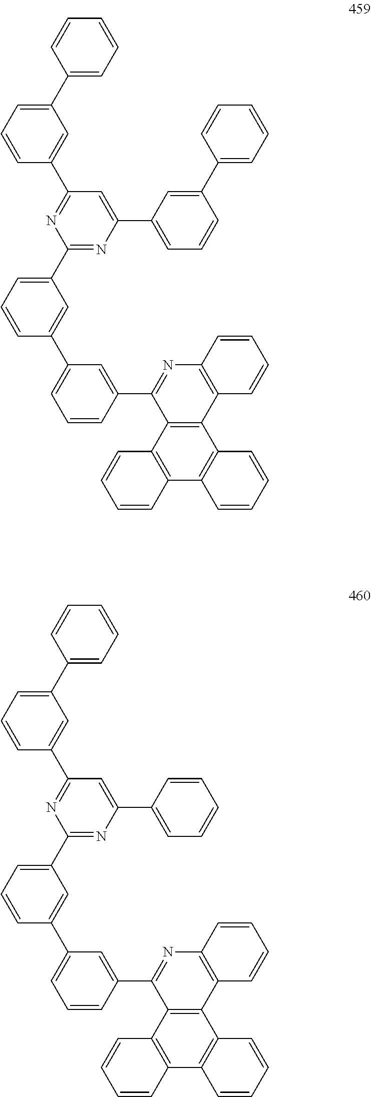 [SMILES: c1ccc(-c2cccc(-c3cc(-c4cccc(-c5ccccc5)c4)nc(-c4cccc(-c5cccc(-c6nc7ccccc7c7c8ccccc8c8ccccc8c67)c5)c4)n3)c2)cc1 ; c1ccc(-c2cccc(-c3cc(-c4ccccc4)nc(-c4cccc(-c5cccc(-c6nc7ccccc7c7c8ccccc8c8ccccc8c67)c5)c4)n3)c2)cc1]

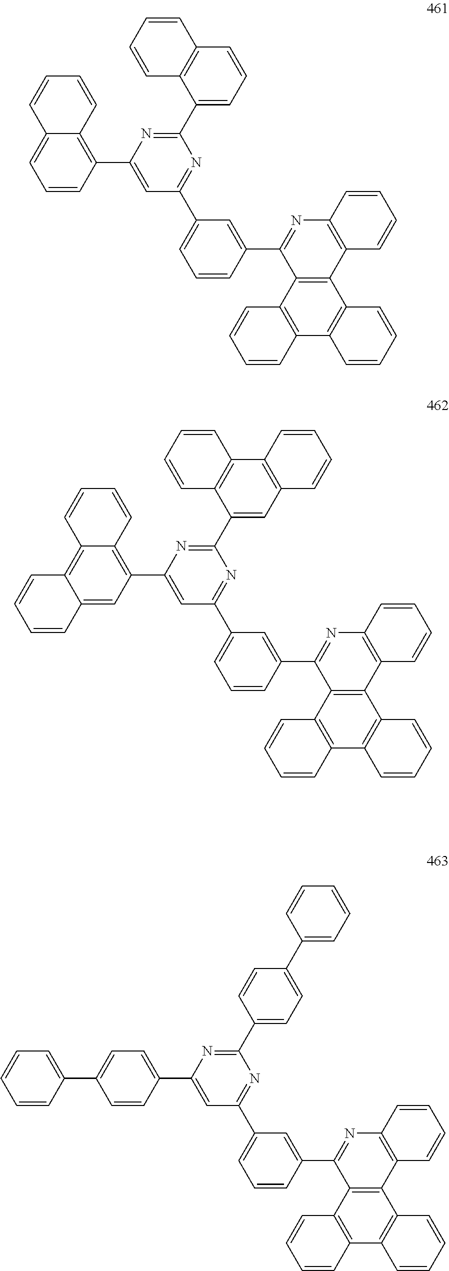 [SMILES: c1cc(-c2cc(-c3cccc4ccccc34)nc(-c3cccc4ccccc34)n2)cc(-c2nc3ccccc3c3c4ccccc4c4ccccc4c23)c1 ; c1cc(-c2cc(-c3cc4ccccc4c4ccccc34)nc(-c3cc4ccccc4c4ccccc34)n2)cc(-c2nc3ccccc3c3c4ccccc4c4ccccc4c23)c1 ; c1ccc(-c2ccc(-c3cc(-c4cccc(-c5nc6ccccc6c6c7ccccc7c7ccccc7c56)c4)nc(-c4ccc(-c5ccccc5)cc4)n3)cc2)cc1]

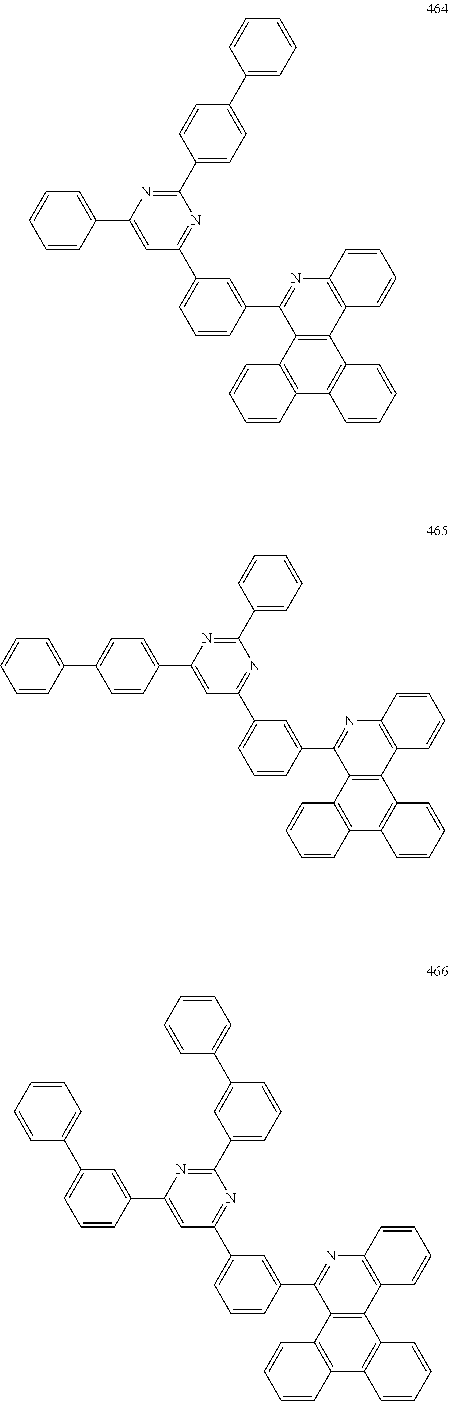 [SMILES: c1ccc(-c2ccc(-c3nc(-c4ccccc4)cc(-c4cccc(-c5nc6ccccc6c6c7ccccc7c7ccccc7c56)c4)n3)cc2)cc1 ; c1ccc(-c2ccc(-c3cc(-c4cccc(-c5nc6ccccc6c6c7ccccc7c7ccccc7c56)c4)nc(-c4ccccc4)n3)cc2)cc1 ; c1ccc(-c2cccc(-c3cc(-c4cccc(-c5nc6ccccc6c6c7ccccc7c7ccccc7c56)c4)nc(-c4cccc(-c5ccccc5)c4)n3)c2)cc1]

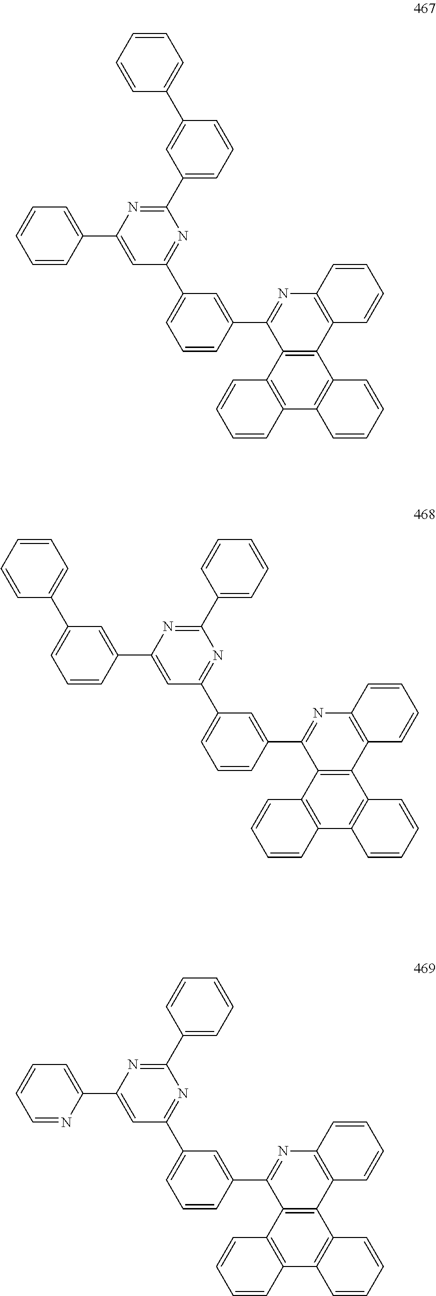 [SMILES: c1ccc(-c2cccc(-c3nc(-c4ccccc4)cc(-c4cccc(-c5nc6ccccc6c6c7ccccc7c7ccccc7c56)c4)n3)c2)cc1 ; c1ccc(-c2cccc(-c3cc(-c4cccc(-c5nc6ccccc6c6c7ccccc7c7ccccc7c56)c4)nc(-c4ccccc4)n3)c2)cc1 ; c1ccc(-c2nc(-c3cccc(-c4nc5ccccc5c5c6ccccc6c6ccccc6c45)c3)cc(-c3ccccn3)n2)cc1]

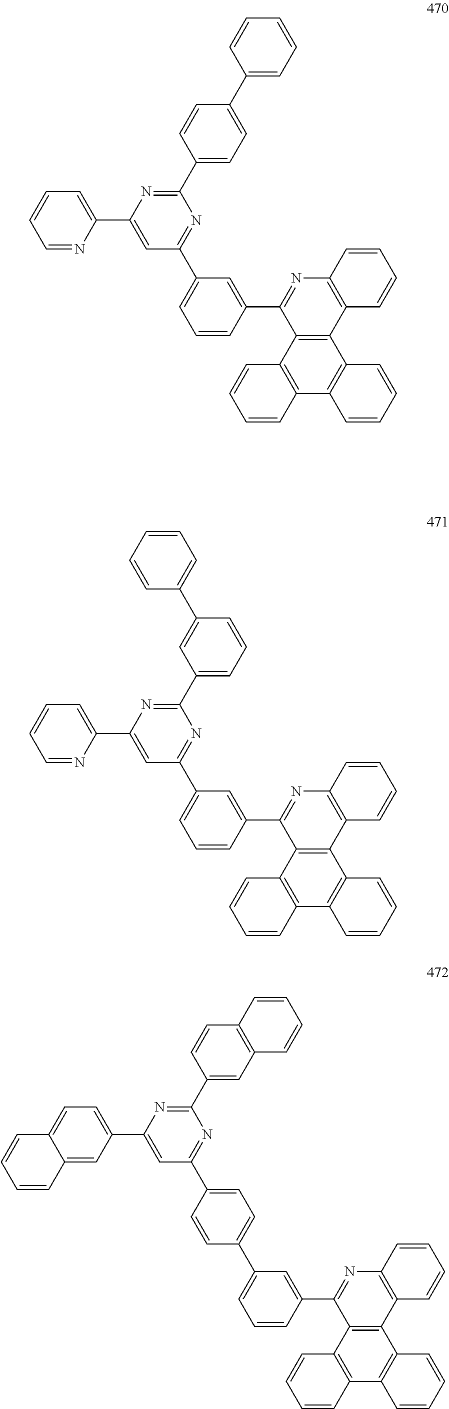 [SMILES: c1ccc(-c2ccc(-c3nc(-c4cccc(-c5nc6ccccc6c6c7ccccc7c7ccccc7c56)c4)cc(-c4ccccn4)n3)cc2)cc1 ; c1ccc(-c2cccc(-c3nc(-c4cccc(-c5nc6ccccc6c6c7ccccc7c7ccccc7c56)c4)cc(-c4ccccn4)n3)c2)cc1 ; c1cc(-c2ccc(-c3cc(-c4ccc5ccccc5c4)nc(-c4ccc5ccccc5c4)n3)cc2)cc(-c2nc3ccccc3c3c4ccccc4c4ccccc4c23)c1]

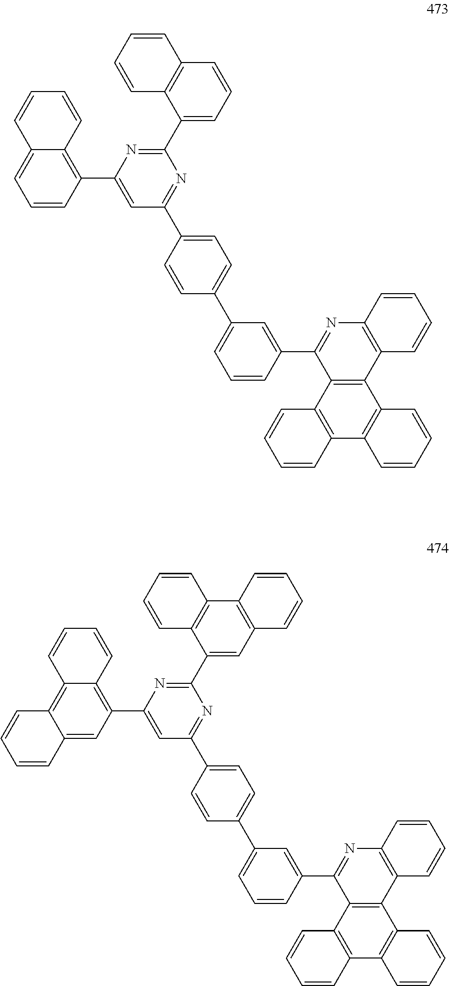 [SMILES: c1cc(-c2ccc(-c3cc(-c4cccc5ccccc45)nc(-c4cccc5ccccc45)n3)cc2)cc(-c2nc3ccccc3c3c4ccccc4c4ccccc4c23)c1 ; c1cc(-c2ccc(-c3cc(-c4cc5ccccc5c5ccccc45)nc(-c4cc5ccccc5c5ccccc45)n3)cc2)cc(-c2nc3ccccc3c3c4ccccc4c4ccccc4c23)c1]

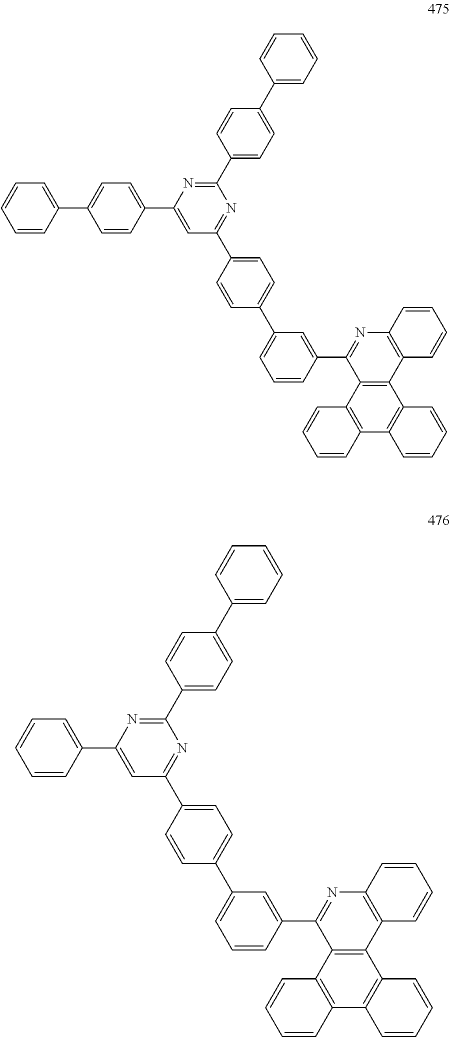 [SMILES: c1ccc(-c2ccc(-c3cc(-c4ccc(-c5cccc(-c6nc7ccccc7c7c8ccccc8c8ccccc8c67)c5)cc4)nc(-c4ccc(-c5ccccc5)cc4)n3)cc2)cc1 ; c1ccc(-c2ccc(-c3nc(-c4ccccc4)cc(-c4ccc(-c5cccc(-c6nc7ccccc7c7c8ccccc8c8ccccc8c67)c5)cc4)n3)cc2)cc1]

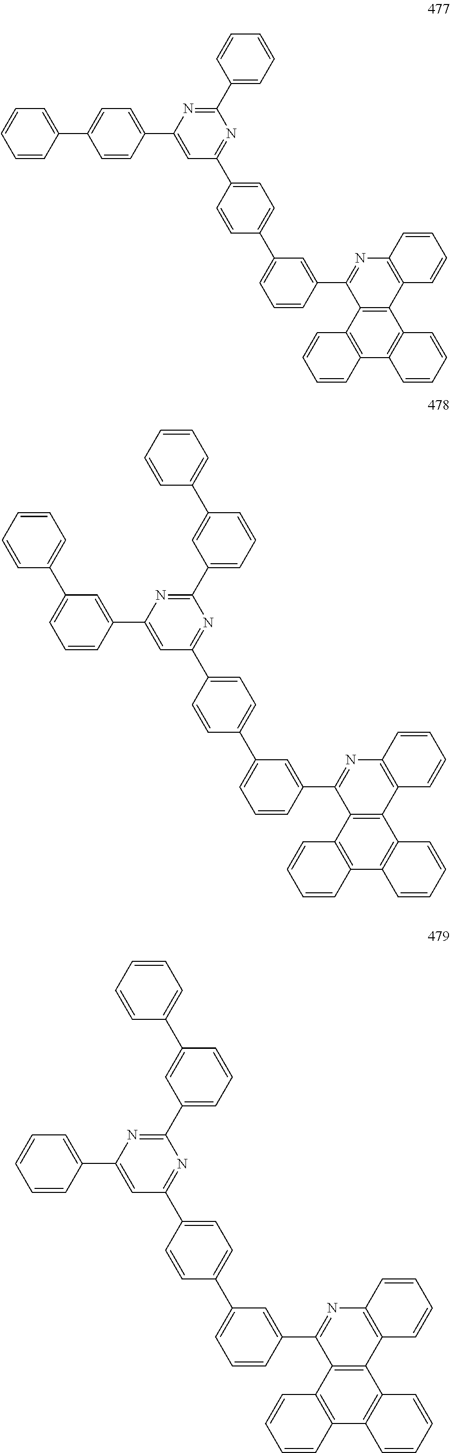 [SMILES: c1ccc(-c2ccc(-c3cc(-c4ccc(-c5cccc(-c6nc7ccccc7c7c8ccccc8c8ccccc8c67)c5)cc4)nc(-c4ccccc4)n3)cc2)cc1 ; c1ccc(-c2cccc(-c3cc(-c4ccc(-c5cccc(-c6nc7ccccc7c7c8ccccc8c8ccccc8c67)c5)cc4)nc(-c4cccc(-c5ccccc5)c4)n3)c2)cc1 ; c1ccc(-c2cccc(-c3nc(-c4ccccc4)cc(-c4ccc(-c5cccc(-c6nc7ccccc7c7c8ccccc8c8ccccc8c67)c5)cc4)n3)c2)cc1]

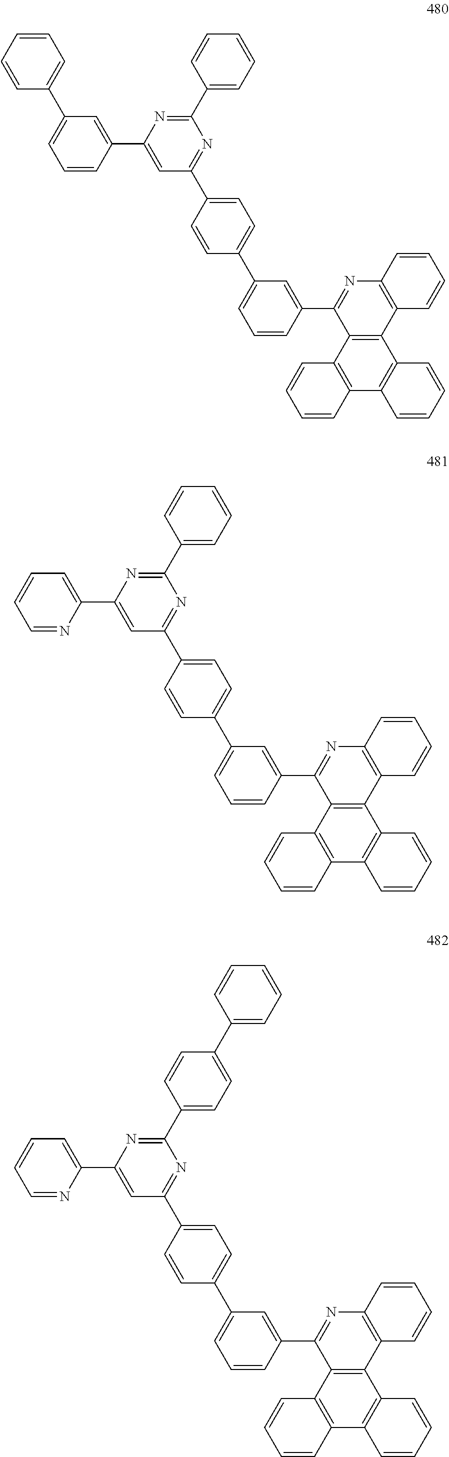 [SMILES: c1ccc(-c2cccc(-c3cc(-c4ccc(-c5cccc(-c6nc7ccccc7c7c8ccccc8c8ccccc8c67)c5)cc4)nc(-c4ccccc4)n3)c2)cc1 ; c1ccc(-c2nc(-c3ccc(-c4cccc(-c5nc6ccccc6c6c7ccccc7c7ccccc7c56)c4)cc3)cc(-c3ccccn3)n2)cc1 ; c1ccc(-c2ccc(-c3nc(-c4ccc(-c5cccc(-c6nc7ccccc7c7c8ccccc8c8ccccc8c67)c5)cc4)cc(-c4ccccn4)n3)cc2)cc1]

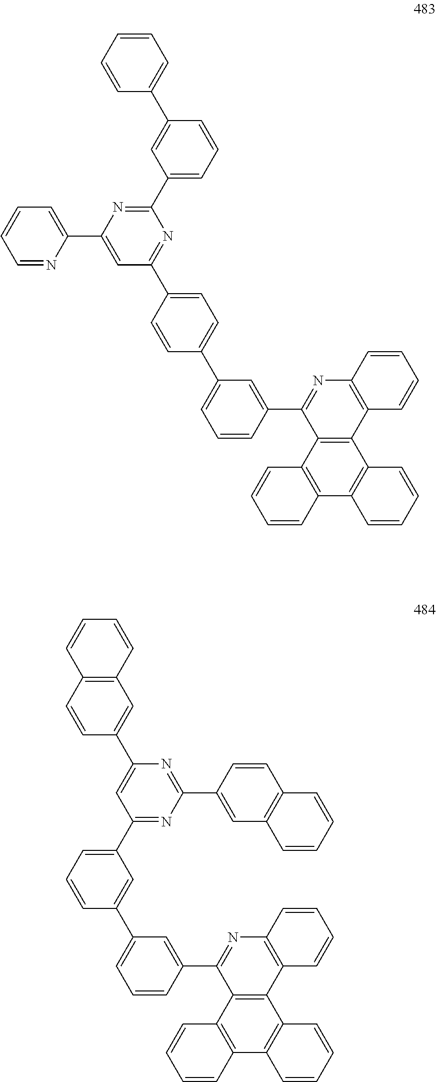 [SMILES: c1ccc(-c2cccc(-c3nc(-c4ccc(-c5cccc(-c6nc7ccccc7c7c8ccccc8c8ccccc8c67)c5)cc4)cc(-c4ccccn4)n3)c2)cc1 ; c1cc(-c2cccc(-c3nc4ccccc4c4c5ccccc5c5ccccc5c34)c2)cc(-c2cc(-c3ccc4ccccc4c3)nc(-c3ccc4ccccc4c3)n2)c1]

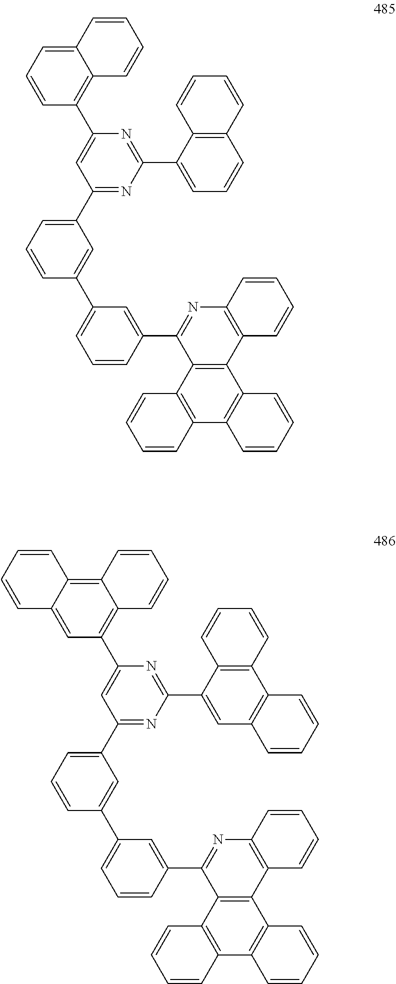 [SMILES: c1cc(-c2cccc(-c3nc4ccccc4c4c5ccccc5c5ccccc5c34)c2)cc(-c2cc(-c3cccc4ccccc34)nc(-c3cccc4ccccc34)n2)c1 ; c1cc(-c2cccc(-c3nc4ccccc4c4c5ccccc5c5ccccc5c34)c2)cc(-c2cc(-c3cc4ccccc4c4ccccc34)nc(-c3cc4ccccc4c4ccccc34)n2)c1]

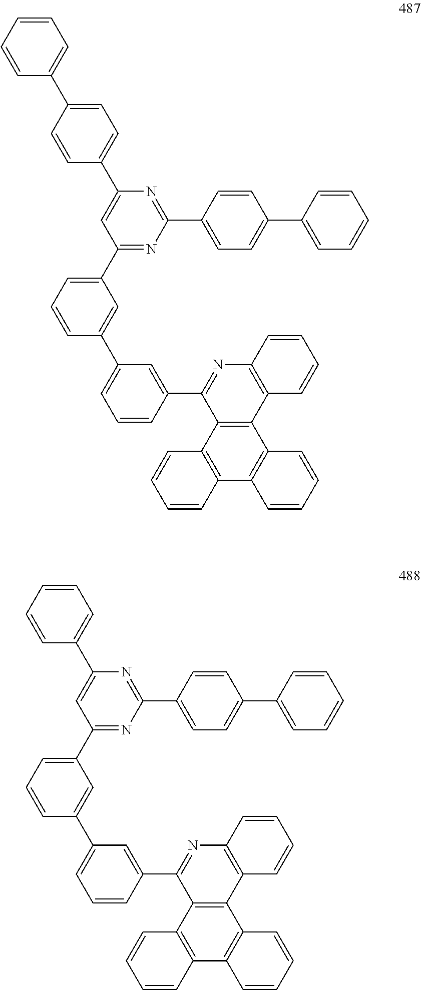 [SMILES: c1ccc(-c2ccc(-c3cc(-c4cccc(-c5cccc(-c6nc7ccccc7c7c8ccccc8c8ccccc8c67)c5)c4)nc(-c4ccc(-c5ccccc5)cc4)n3)cc2)cc1 ; c1ccc(-c2ccc(-c3nc(-c4ccccc4)cc(-c4cccc(-c5cccc(-c6nc7ccccc7c7c8ccccc8c8ccccc8c67)c5)c4)n3)cc2)cc1]

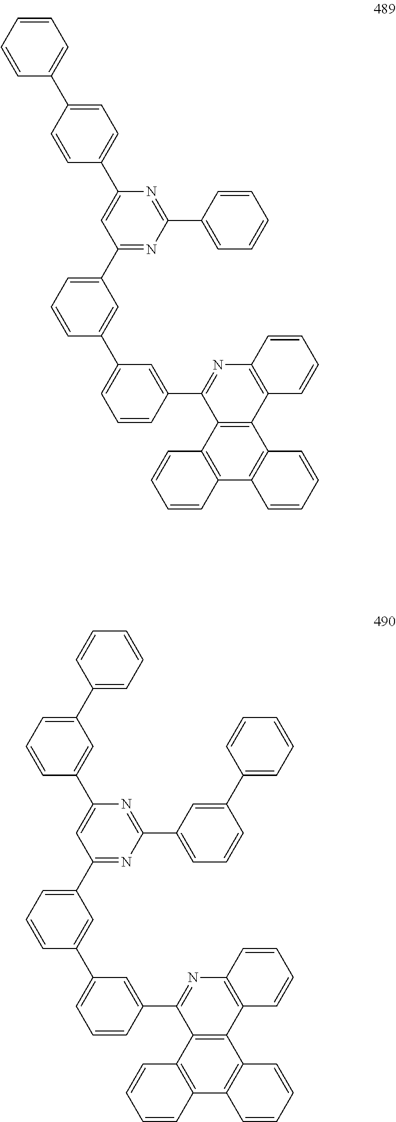 [SMILES: c1ccc(-c2ccc(-c3cc(-c4cccc(-c5cccc(-c6nc7ccccc7c7c8ccccc8c8ccccc8c67)c5)c4)nc(-c4ccccc4)n3)cc2)cc1 ; c1ccc(-c2cccc(-c3cc(-c4cccc(-c5cccc(-c6nc7ccccc7c7c8ccccc8c8ccccc8c67)c5)c4)nc(-c4cccc(-c5ccccc5)c4)n3)c2)cc1]

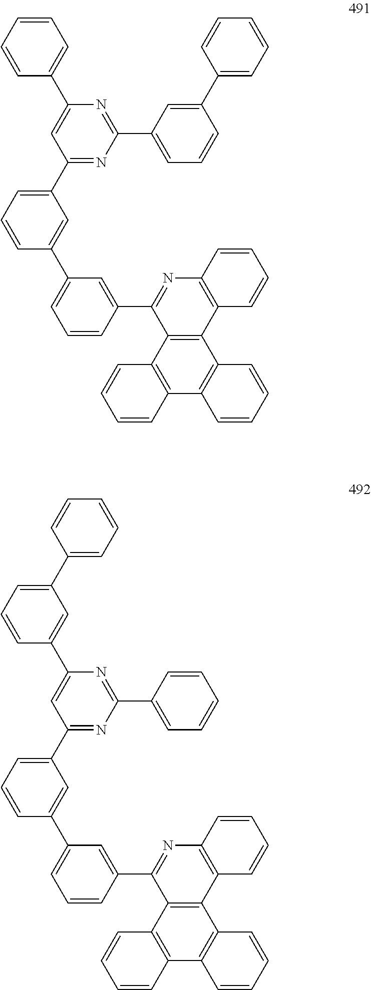 [SMILES: c1ccc(-c2cccc(-c3nc(-c4ccccc4)cc(-c4cccc(-c5cccc(-c6nc7ccccc7c7c8ccccc8c8ccccc8c67)c5)c4)n3)c2)cc1 ; c1ccc(-c2cccc(-c3cc(-c4cccc(-c5cccc(-c6nc7ccccc7c7c8ccccc8c8ccccc8c67)c5)c4)nc(-c4ccccc4)n3)c2)cc1]

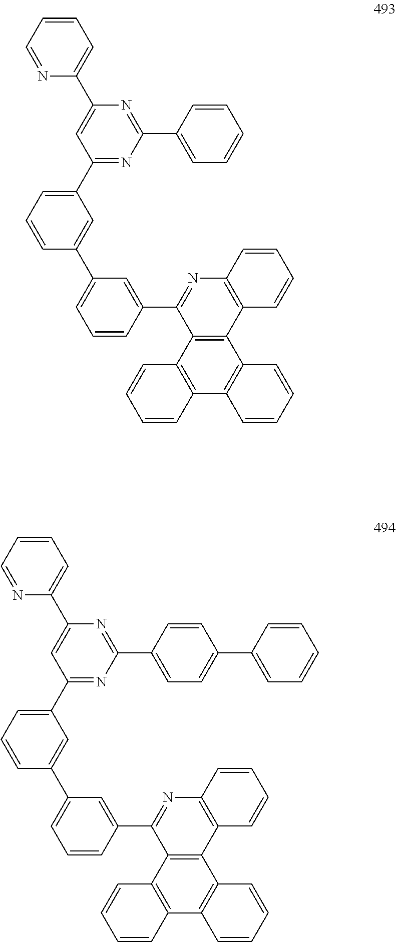 [SMILES: c1ccc(-c2nc(-c3cccc(-c4cccc(-c5nc6ccccc6c6c7ccccc7c7ccccc7c56)c4)c3)cc(-c3ccccn3)n2)cc1 ; c1ccc(-c2ccc(-c3nc(-c4cccc(-c5cccc(-c6nc7ccccc7c7c8ccccc8c8ccccc8c67)c5)c4)cc(-c4ccccn4)n3)cc2)cc1]

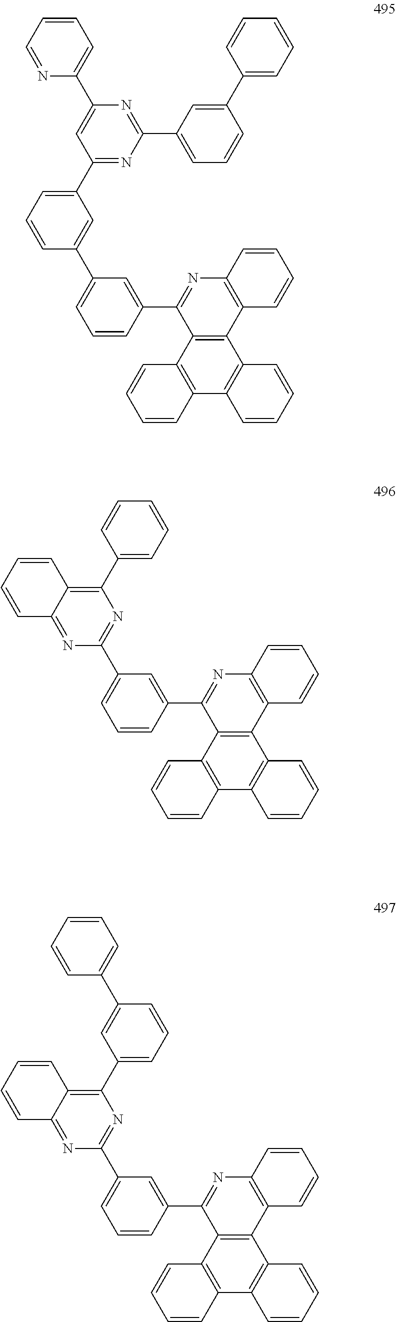 [SMILES: c1ccc(-c2cccc(-c3nc(-c4cccc(-c5cccc(-c6nc7ccccc7c7c8ccccc8c8ccccc8c67)c5)c4)cc(-c4ccccn4)n3)c2)cc1 ; c1ccc(-c2nc(-c3cccc(-c4nc5ccccc5c5c6ccccc6c6ccccc6c45)c3)nc3ccccc23)cc1 ; c1ccc(-c2cccc(-c3nc(-c4cccc(-c5nc6ccccc6c6c7ccccc7c7ccccc7c56)c4)nc4ccccc34)c2)cc1]

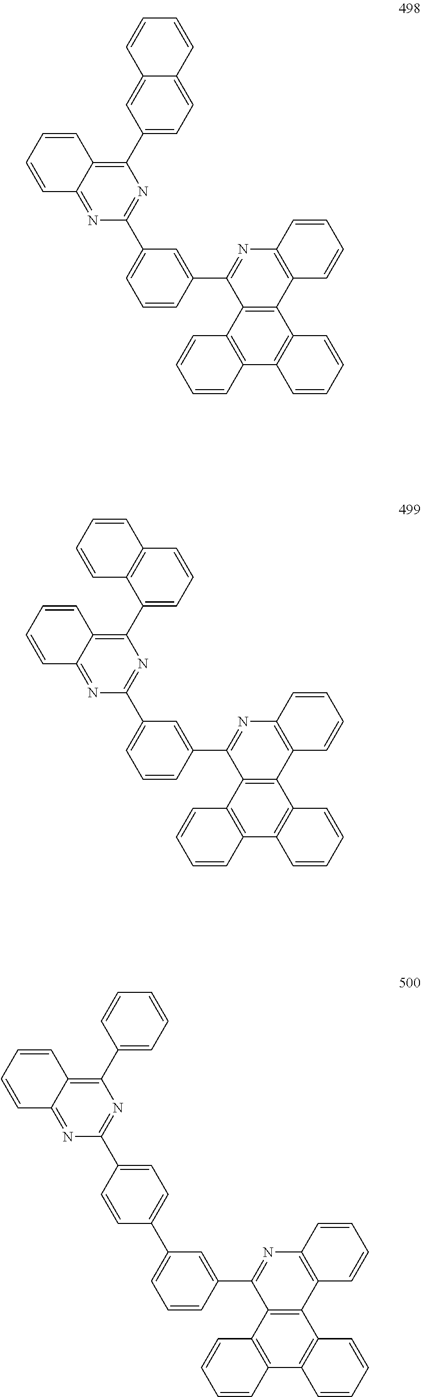 [SMILES: c1cc(-c2nc(-c3ccc4ccccc4c3)c3ccccc3n2)cc(-c2nc3ccccc3c3c4ccccc4c4ccccc4c23)c1 ; c1cc(-c2nc(-c3cccc4ccccc34)c3ccccc3n2)cc(-c2nc3ccccc3c3c4ccccc4c4ccccc4c23)c1 ; c1ccc(-c2nc(-c3ccc(-c4cccc(-c5nc6ccccc6c6c7ccccc7c7ccccc7c56)c4)cc3)nc3ccccc23)cc1]

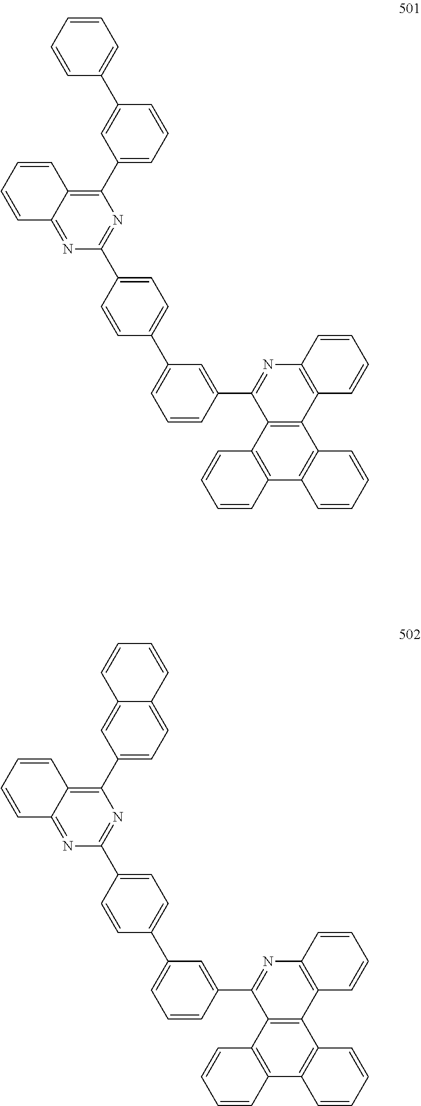 [SMILES: c1ccc(-c2cccc(-c3nc(-c4ccc(-c5cccc(-c6nc7ccccc7c7c8ccccc8c8ccccc8c67)c5)cc4)nc4ccccc34)c2)cc1 ; c1cc(-c2ccc(-c3nc(-c4ccc5ccccc5c4)c4ccccc4n3)cc2)cc(-c2nc3ccccc3c3c4ccccc4c4ccccc4c23)c1]

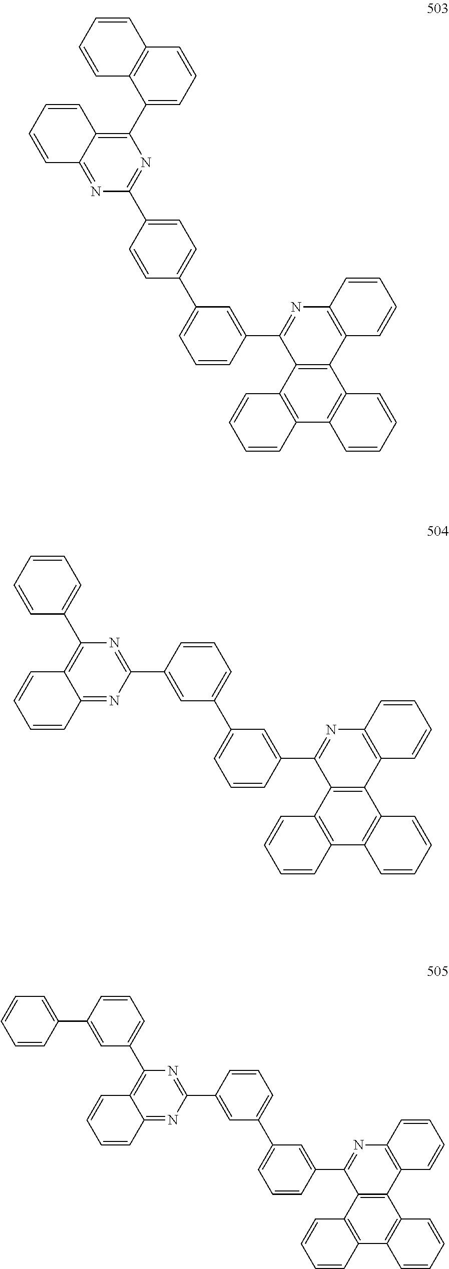 [SMILES: c1cc(-c2ccc(-c3nc(-c4cccc5ccccc45)c4ccccc4n3)cc2)cc(-c2nc3ccccc3c3c4ccccc4c4ccccc4c23)c1 ; c1ccc(-c2nc(-c3cccc(-c4cccc(-c5nc6ccccc6c6c7ccccc7c7ccccc7c56)c4)c3)nc3ccccc23)cc1 ; c1ccc(-c2cccc(-c3nc(-c4cccc(-c5cccc(-c6nc7ccccc7c7c8ccccc8c8ccccc8c67)c5)c4)nc4ccccc34)c2)cc1]

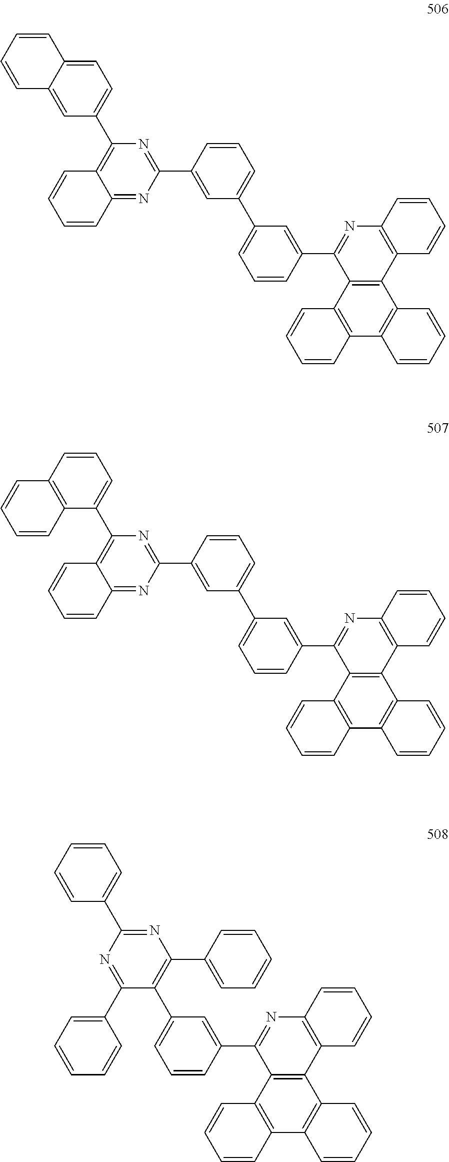 [SMILES: c1cc(-c2cccc(-c3nc4ccccc4c4c5ccccc5c5ccccc5c34)c2)cc(-c2nc(-c3ccc4ccccc4c3)c3ccccc3n2)c1 ; c1cc(-c2cccc(-c3nc4ccccc4c4c5ccccc5c5ccccc5c34)c2)cc(-c2nc(-c3cccc4ccccc34)c3ccccc3n2)c1 ; c1ccc(-c2nc(-c3ccccc3)c(-c3cccc(-c4nc5ccccc5c5c6ccccc6c6ccccc6c45)c3)c(-c3ccccc3)n2)cc1]

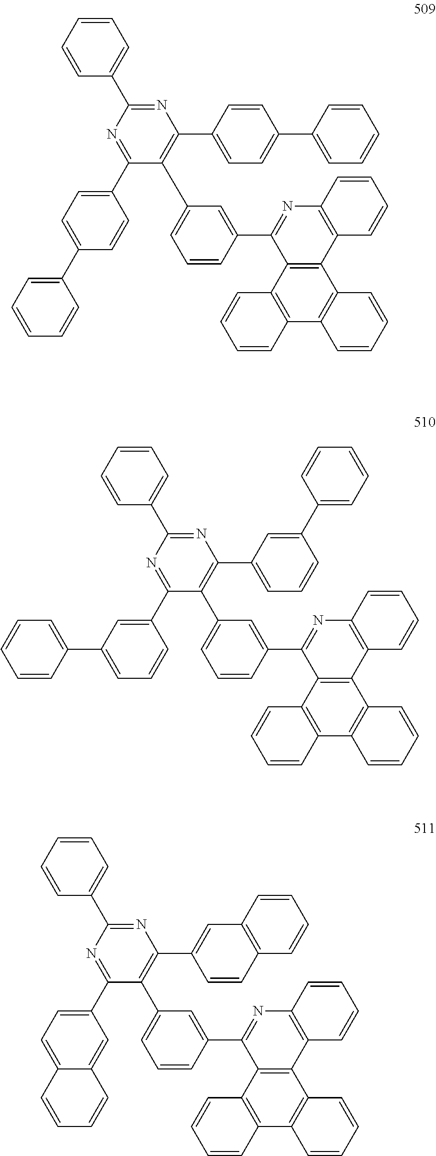 [SMILES: c1ccc(-c2ccc(-c3nc(-c4ccccc4)nc(-c4ccc(-c5ccccc5)cc4)c3-c3cccc(-c4nc5ccccc5c5c6ccccc6c6ccccc6c45)c3)cc2)cc1 ; c1ccc(-c2cccc(-c3nc(-c4ccccc4)nc(-c4cccc(-c5ccccc5)c4)c3-c3cccc(-c4nc5ccccc5c5c6ccccc6c6ccccc6c45)c3)c2)cc1 ; c1ccc(-c2nc(-c3ccc4ccccc4c3)c(-c3cccc(-c4nc5ccccc5c5c6ccccc6c6ccccc6c45)c3)c(-c3ccc4ccccc4c3)n2)cc1]

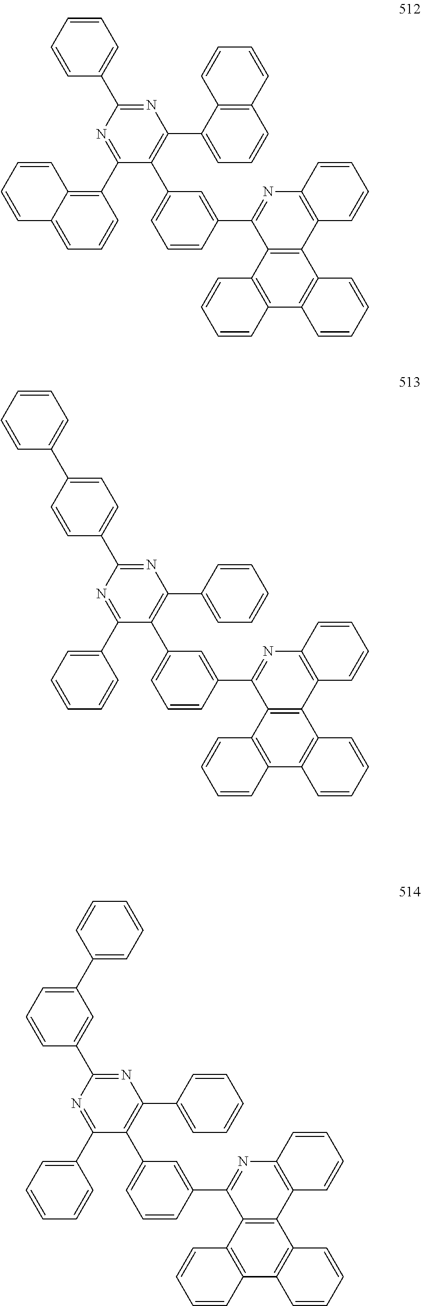 [SMILES: c1ccc(-c2nc(-c3cccc4ccccc34)c(-c3cccc(-c4nc5ccccc5c5c6ccccc6c6ccccc6c45)c3)c(-c3cccc4ccccc34)n2)cc1 ; c1ccc(-c2ccc(-c3nc(-c4ccccc4)c(-c4cccc(-c5nc6ccccc6c6c7ccccc7c7ccccc7c56)c4)c(-c4ccccc4)n3)cc2)cc1 ; c1ccc(-c2cccc(-c3nc(-c4ccccc4)c(-c4cccc(-c5nc6ccccc6c6c7ccccc7c7ccccc7c56)c4)c(-c4ccccc4)n3)c2)cc1]

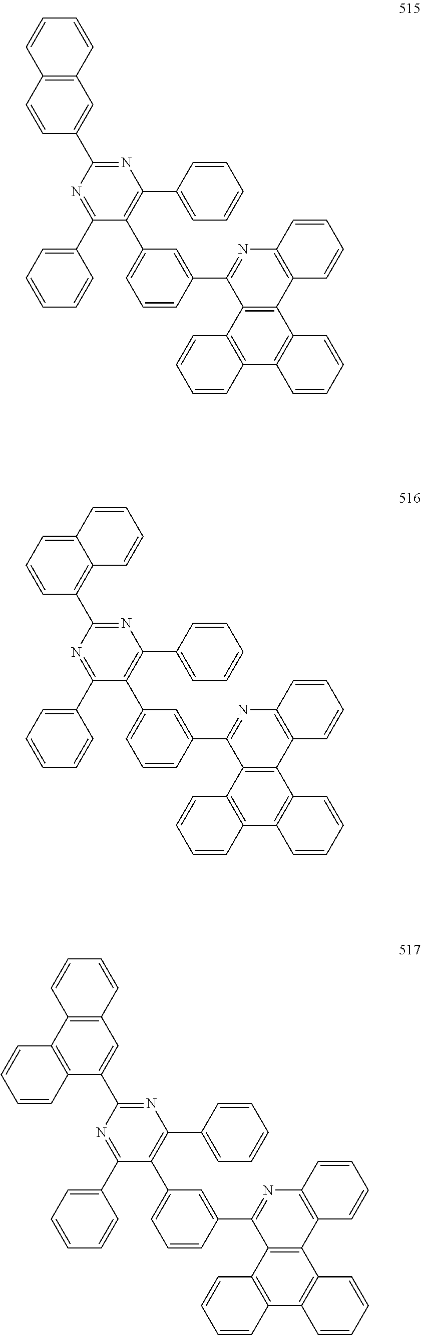 [SMILES: c1ccc(-c2nc(-c3ccc4ccccc4c3)nc(-c3ccccc3)c2-c2cccc(-c3nc4ccccc4c4c5ccccc5c5ccccc5c34)c2)cc1 ; c1ccc(-c2nc(-c3cccc4ccccc34)nc(-c3ccccc3)c2-c2cccc(-c3nc4ccccc4c4c5ccccc5c5ccccc5c34)c2)cc1 ; c1ccc(-c2nc(-c3cc4ccccc4c4ccccc34)nc(-c3ccccc3)c2-c2cccc(-c3nc4ccccc4c4c5ccccc5c5ccccc5c34)c2)cc1]

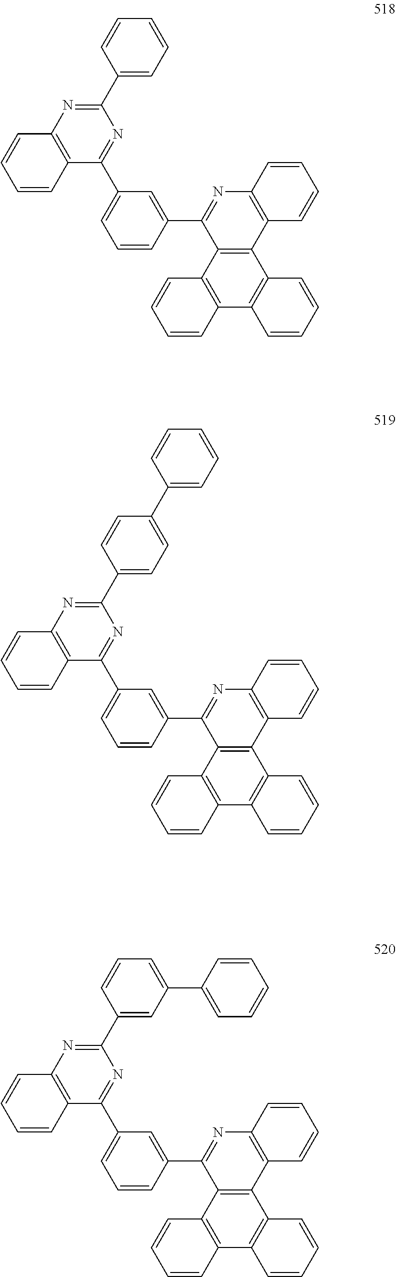 [SMILES: c1ccc(-c2nc(-c3cccc(-c4nc5ccccc5c5c6ccccc6c6ccccc6c45)c3)c3ccccc3n2)cc1 ; c1ccc(-c2ccc(-c3nc(-c4cccc(-c5nc6ccccc6c6c7ccccc7c7ccccc7c56)c4)c4ccccc4n3)cc2)cc1 ; c1ccc(-c2cccc(-c3nc(-c4cccc(-c5nc6ccccc6c6c7ccccc7c7ccccc7c56)c4)c4ccccc4n3)c2)cc1]

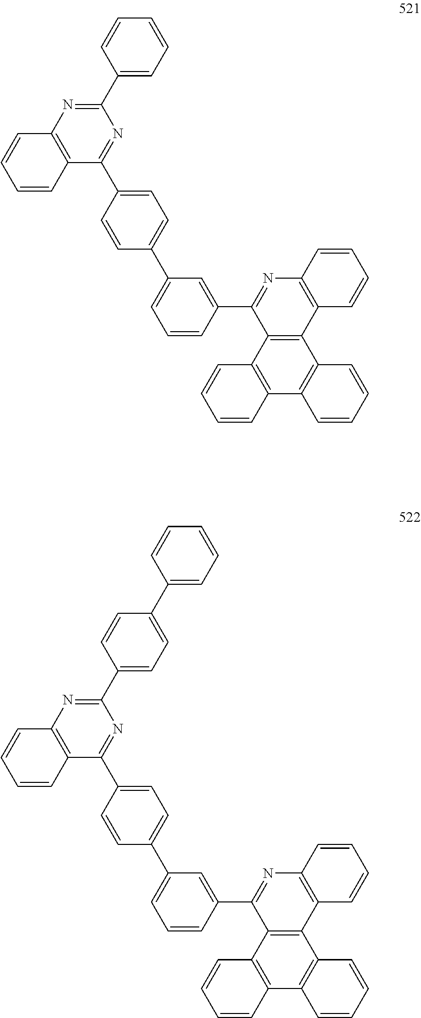 [SMILES: c1ccc(-c2nc(-c3ccc(-c4cccc(-c5nc6ccccc6c6c7ccccc7c7ccccc7c56)c4)cc3)c3ccccc3n2)cc1 ; c1ccc(-c2ccc(-c3nc(-c4ccc(-c5cccc(-c6nc7ccccc7c7c8ccccc8c8ccccc8c67)c5)cc4)c4ccccc4n3)cc2)cc1]

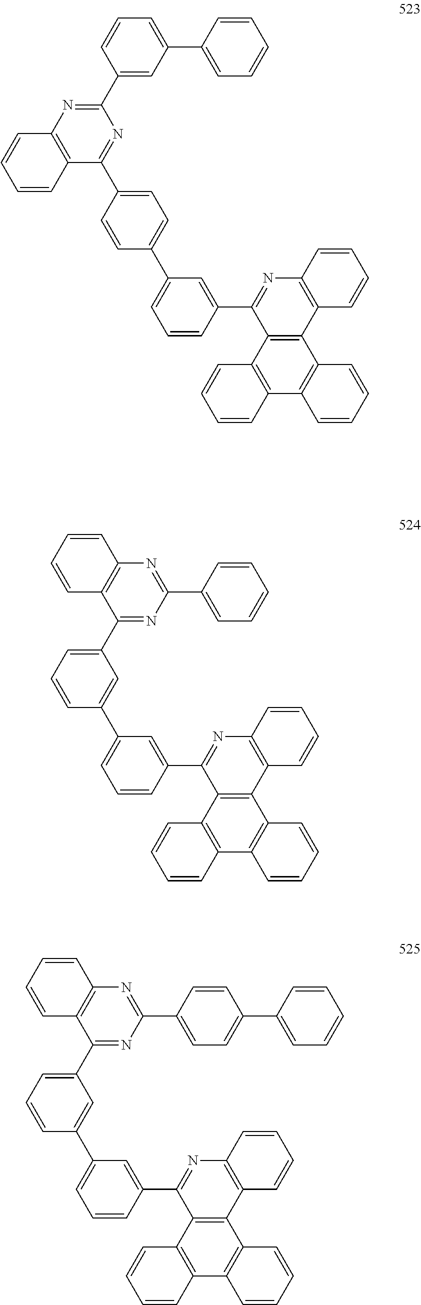 [SMILES: c1ccc(-c2cccc(-c3nc(-c4ccc(-c5cccc(-c6nc7ccccc7c7c8ccccc8c8ccccc8c67)c5)cc4)c4ccccc4n3)c2)cc1 ; c1ccc(-c2nc(-c3cccc(-c4cccc(-c5nc6ccccc6c6c7ccccc7c7ccccc7c56)c4)c3)c3ccccc3n2)cc1 ; c1ccc(-c2ccc(-c3nc(-c4cccc(-c5cccc(-c6nc7ccccc7c7c8ccccc8c8ccccc8c67)c5)c4)c4ccccc4n3)cc2)cc1]

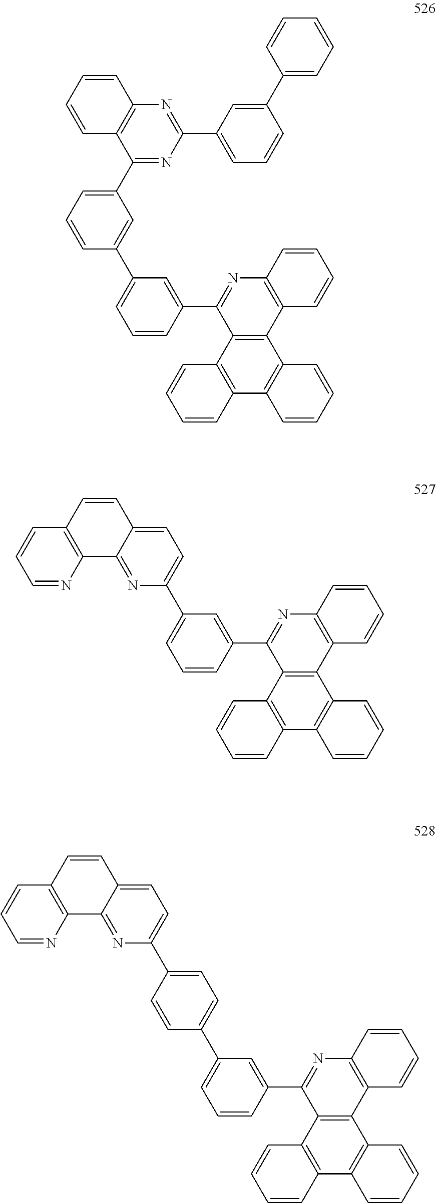 [SMILES: c1ccc(-c2cccc(-c3nc(-c4cccc(-c5cccc(-c6nc7ccccc7c7c8ccccc8c8ccccc8c67)c5)c4)c4ccccc4n3)c2)cc1 ; c1cc(-c2ccc3ccc4cccnc4c3n2)cc(-c2nc3ccccc3c3c4ccccc4c4ccccc4c23)c1 ; c1cc(-c2ccc(-c3ccc4ccc5cccnc5c4n3)cc2)cc(-c2nc3ccccc3c3c4ccccc4c4ccccc4c23)c1]

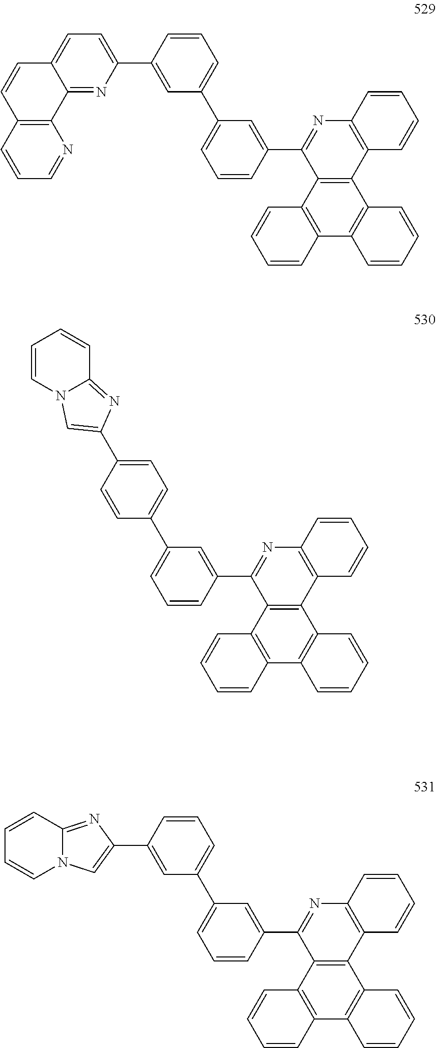 [SMILES: c1cc(-c2cccc(-c3nc4ccccc4c4c5ccccc5c5ccccc5c34)c2)cc(-c2ccc3ccc4cccnc4c3n2)c1 ; c1cc(-c2ccc(-c3cn4ccccc4n3)cc2)cc(-c2nc3ccccc3c3c4ccccc4c4ccccc4c23)c1 ; c1cc(-c2cccc(-c3nc4ccccc4c4c5ccccc5c5ccccc5c34)c2)cc(-c2cn3ccccc3n2)c1]

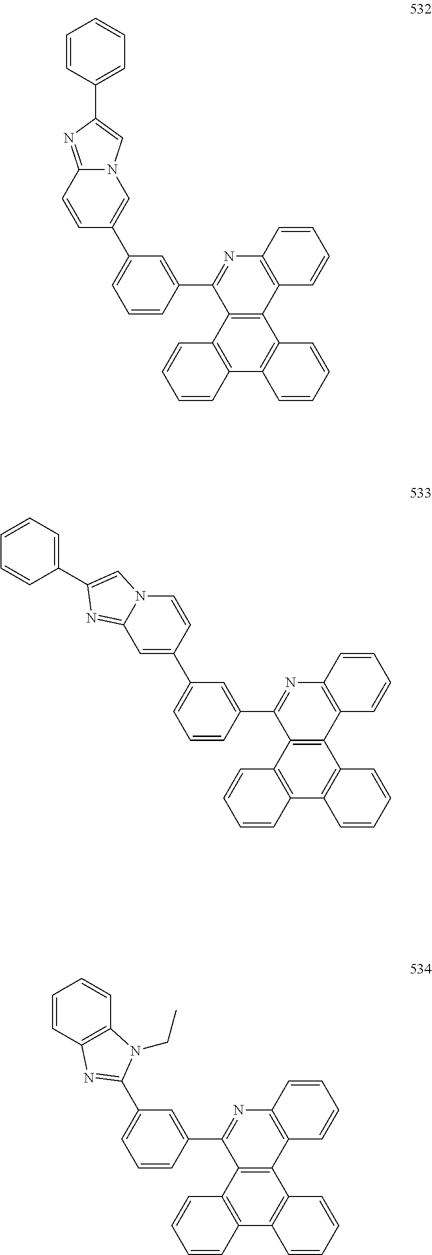 [SMILES: c1ccc(-c2cn3cc(-c4cccc(-c5nc6ccccc6c6c7ccccc7c7ccccc7c56)c4)ccc3n2)cc1 ; c1ccc(-c2cn3ccc(-c4cccc(-c5nc6ccccc6c6c7ccccc7c7ccccc7c56)c4)cc3n2)cc1 ; CCn1c(-c2cccc(-c3nc4ccccc4c4c5ccccc5c5ccccc5c34)c2)nc2ccccc21]

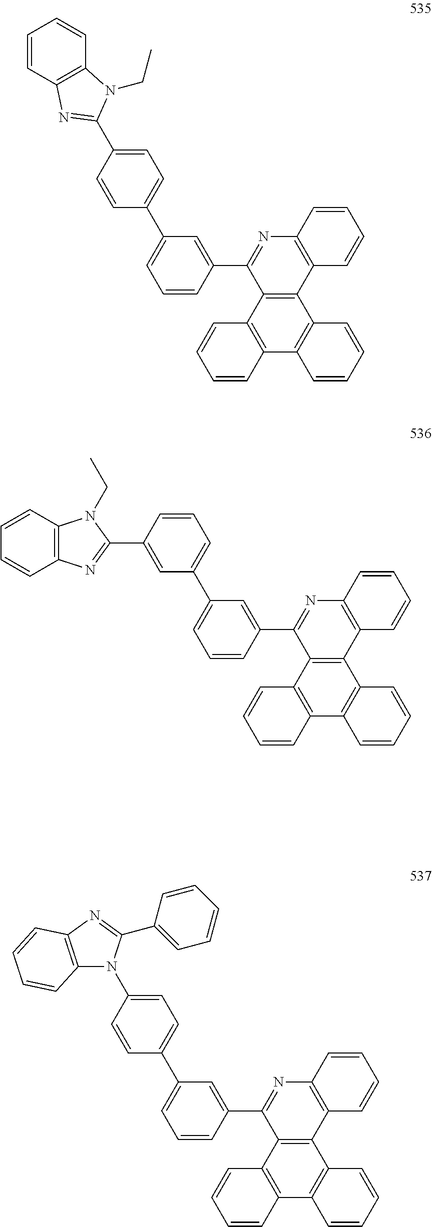 [SMILES: CCn1c(-c2ccc(-c3cccc(-c4nc5ccccc5c5c6ccccc6c6ccccc6c45)c3)cc2)nc2ccccc21 ; CCn1c(-c2cccc(-c3cccc(-c4nc5ccccc5c5c6ccccc6c6ccccc6c45)c3)c2)nc2ccccc21 ; c1ccc(-c2nc3ccccc3n2-c2ccc(-c3cccc(-c4nc5ccccc5c5c6ccccc6c6ccccc6c45)c3)cc2)cc1]

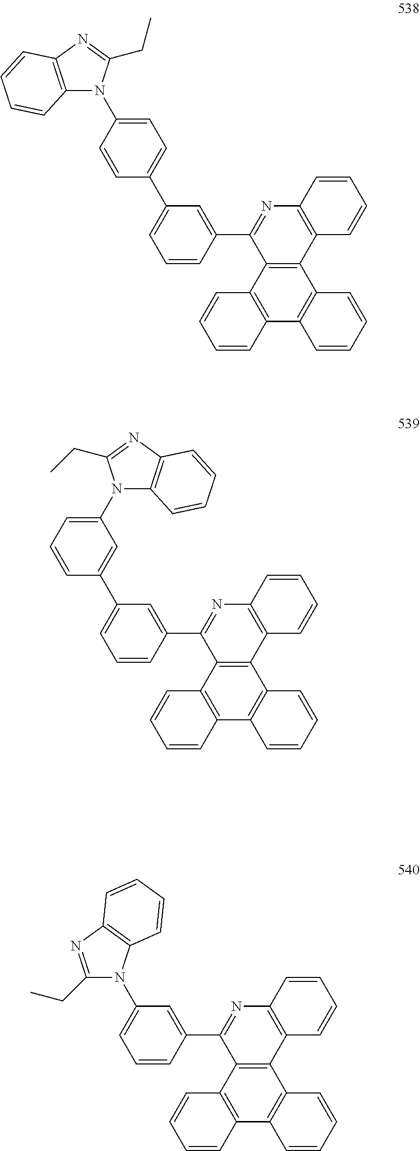 [SMILES: CCc1nc2ccccc2n1-c1ccc(-c2cccc(-c3nc4ccccc4c4c5ccccc5c5ccccc5c34)c2)cc1 ; CCc1nc2ccccc2n1-c1cccc(-c2cccc(-c3nc4ccccc4c4c5ccccc5c5ccccc5c34)c2)c1 ; CCc1nc2ccccc2n1-c1cccc(-c2nc3ccccc3c3c4ccccc4c4ccccc4c23)c1]

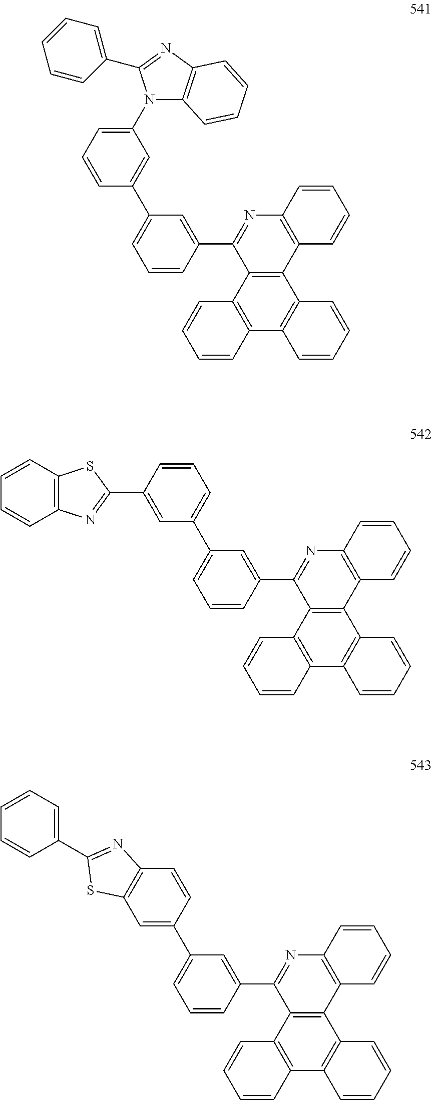 [SMILES: c1ccc(-c2nc3ccccc3n2-c2cccc(-c3cccc(-c4nc5ccccc5c5c6ccccc6c6ccccc6c45)c3)c2)cc1 ; c1cc(-c2cccc(-c3nc4ccccc4c4c5ccccc5c5ccccc5c34)c2)cc(-c2nc3ccccc3s2)c1 ; c1ccc(-c2nc3ccc(-c4cccc(-c5nc6ccccc6c6c7ccccc7c7ccccc7c56)c4)cc3s2)cc1]

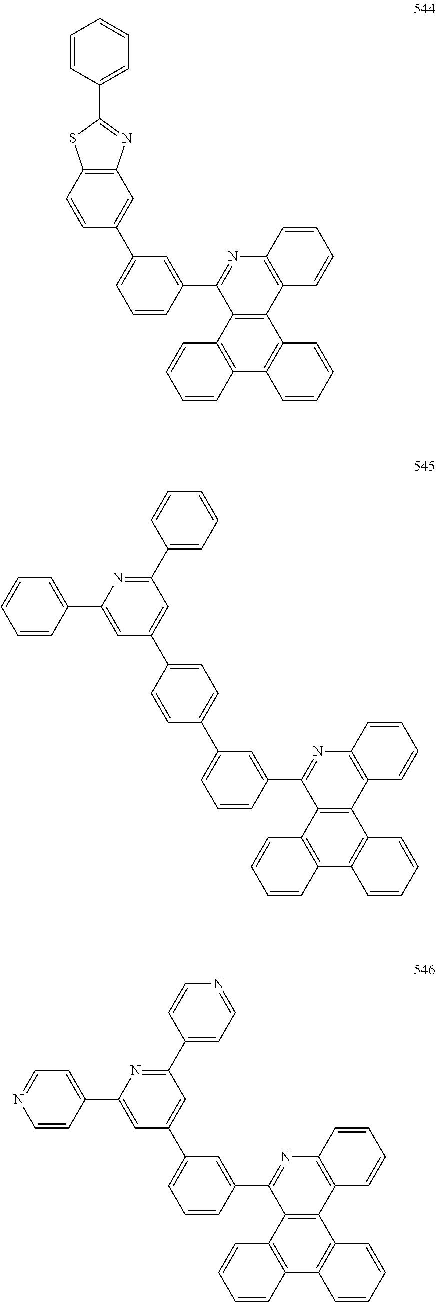 [SMILES: c1ccc(-c2nc3cc(-c4cccc(-c5nc6ccccc6c6c7ccccc7c7ccccc7c56)c4)ccc3s2)cc1 ; c1ccc(-c2cc(-c3ccc(-c4cccc(-c5nc6ccccc6c6c7ccccc7c7ccccc7c56)c4)cc3)cc(-c3ccccc3)n2)cc1 ; c1cc(-c2cc(-c3ccncc3)nc(-c3ccncc3)c2)cc(-c2nc3ccccc3c3c4ccccc4c4ccccc4c23)c1]

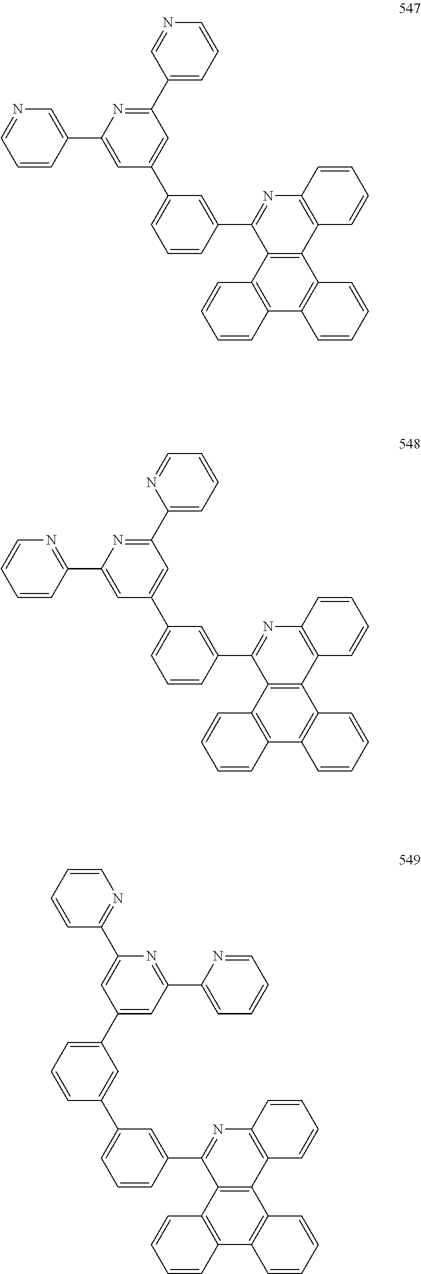 [SMILES: c1cncc(-c2cc(-c3cccc(-c4nc5ccccc5c5c6ccccc6c6ccccc6c45)c3)cc(-c3cccnc3)n2)c1 ; c1ccc(-c2cc(-c3cccc(-c4nc5ccccc5c5c6ccccc6c6ccccc6c45)c3)cc(-c3ccccn3)n2)nc1 ; c1ccc(-c2cc(-c3cccc(-c4cccc(-c5nc6ccccc6c6c7ccccc7c7ccccc7c56)c4)c3)cc(-c3ccccn3)n2)nc1]

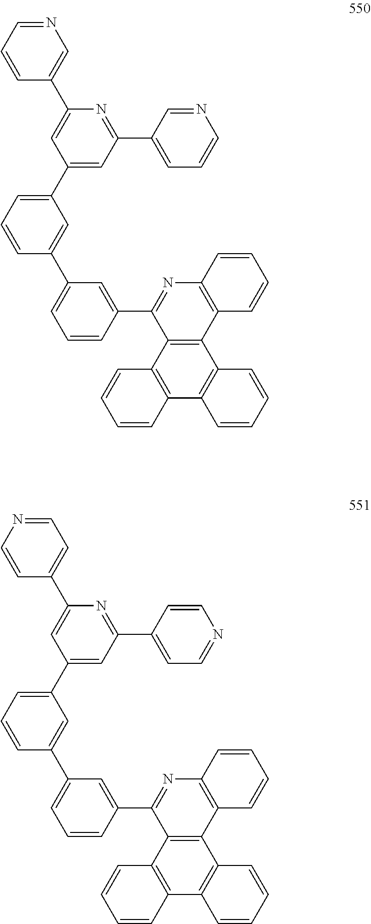 [SMILES: c1cc(-c2cccc(-c3nc4ccccc4c4c5ccccc5c5ccccc5c34)c2)cc(-c2cc(-c3cccnc3)nc(-c3cccnc3)c2)c1 ; c1cc(-c2cccc(-c3nc4ccccc4c4c5ccccc5c5ccccc5c34)c2)cc(-c2cc(-c3ccncc3)nc(-c3ccncc3)c2)c1]

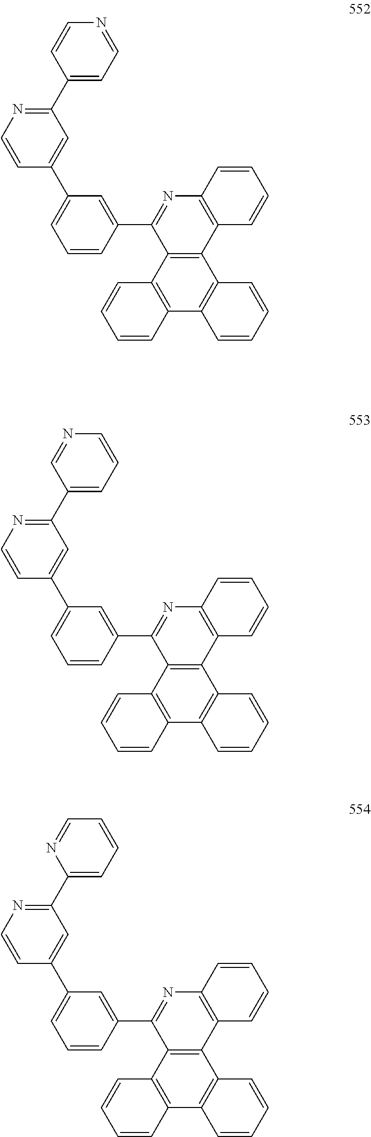 [SMILES: c1cc(-c2ccnc(-c3ccncc3)c2)cc(-c2nc3ccccc3c3c4ccccc4c4ccccc4c23)c1 ; c1cncc(-c2cc(-c3cccc(-c4nc5ccccc5c5c6ccccc6c6ccccc6c45)c3)ccn2)c1 ; c1ccc(-c2cc(-c3cccc(-c4nc5ccccc5c5c6ccccc6c6ccccc6c45)c3)ccn2)nc1]

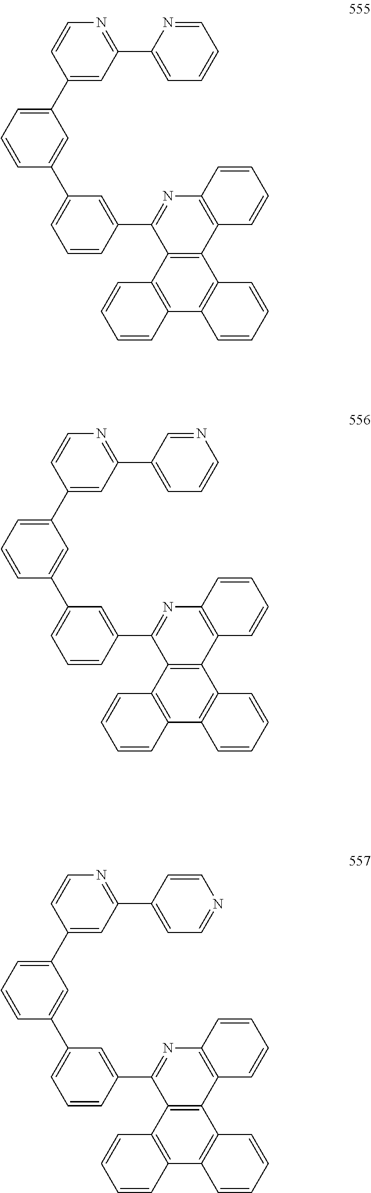 [SMILES: c1ccc(-c2cc(-c3cccc(-c4cccc(-c5nc6ccccc6c6c7ccccc7c7ccccc7c56)c4)c3)ccn2)nc1 ; c1cc(-c2cccc(-c3nc4ccccc4c4c5ccccc5c5ccccc5c34)c2)cc(-c2ccnc(-c3cccnc3)c2)c1 ; c1cc(-c2cccc(-c3nc4ccccc4c4c5ccccc5c5ccccc5c34)c2)cc(-c2ccnc(-c3ccncc3)c2)c1]

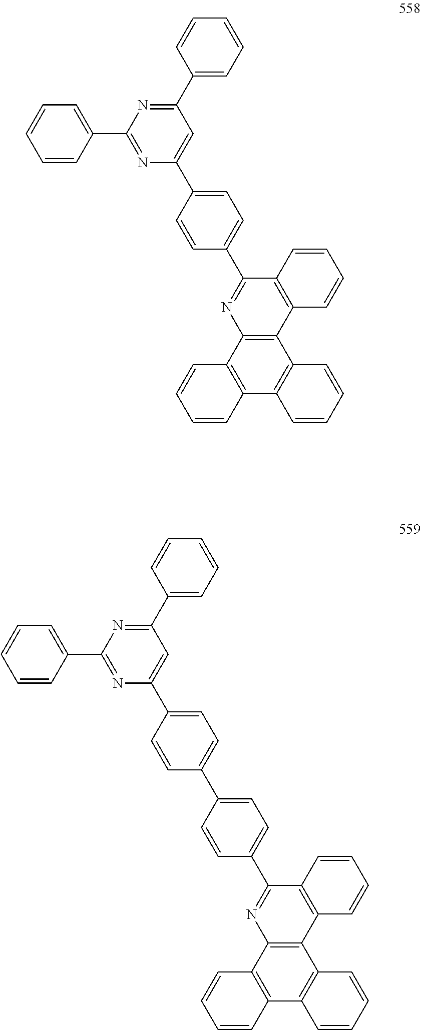 [SMILES: c1ccc(-c2cc(-c3ccc(-c4nc5c6ccccc6c6ccccc6c5c5ccccc45)cc3)nc(-c3ccccc3)n2)cc1 ; c1ccc(-c2cc(-c3ccc(-c4ccc(-c5nc6c7ccccc7c7ccccc7c6c6ccccc56)cc4)cc3)nc(-c3ccccc3)n2)cc1]

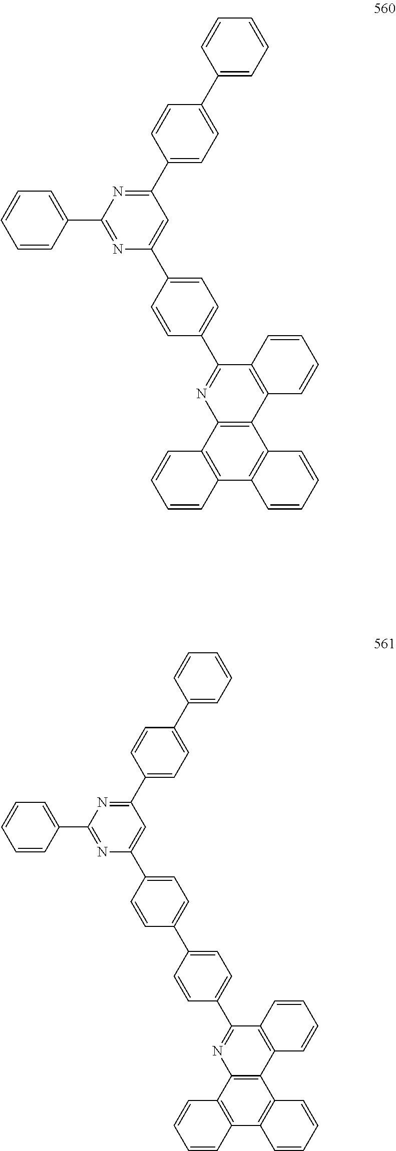 [SMILES: c1ccc(-c2ccc(-c3cc(-c4ccc(-c5nc6c7ccccc7c7ccccc7c6c6ccccc56)cc4)nc(-c4ccccc4)n3)cc2)cc1 ; c1ccc(-c2ccc(-c3cc(-c4ccc(-c5ccc(-c6nc7c8ccccc8c8ccccc8c7c7ccccc67)cc5)cc4)nc(-c4ccccc4)n3)cc2)cc1]

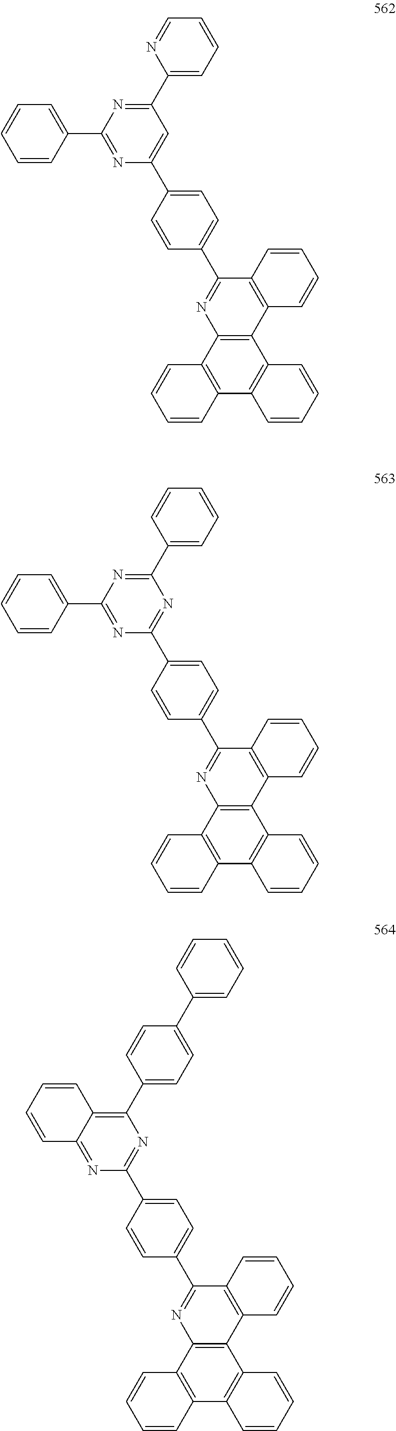 [SMILES: c1ccc(-c2nc(-c3ccc(-c4nc5c6ccccc6c6ccccc6c5c5ccccc45)cc3)cc(-c3ccccn3)n2)cc1 ; c1ccc(-c2nc(-c3ccccc3)nc(-c3ccc(-c4nc5c6ccccc6c6ccccc6c5c5ccccc45)cc3)n2)cc1 ; c1ccc(-c2ccc(-c3nc(-c4ccc(-c5nc6c7ccccc7c7ccccc7c6c6ccccc56)cc4)nc4ccccc34)cc2)cc1]

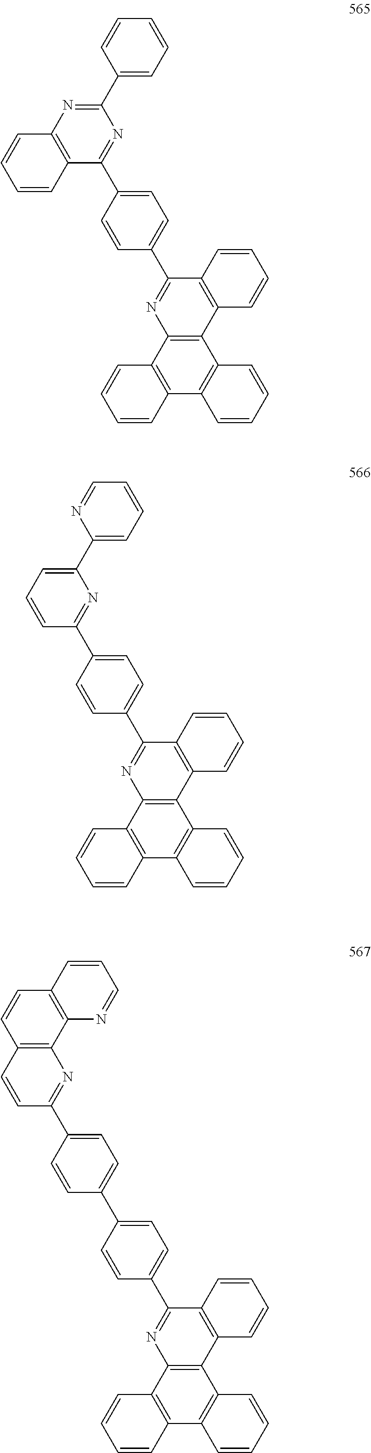 [SMILES: c1ccc(-c2nc(-c3ccc(-c4nc5c6ccccc6c6ccccc6c5c5ccccc45)cc3)c3ccccc3n2)cc1 ; c1ccc(-c2cccc(-c3ccc(-c4nc5c6ccccc6c6ccccc6c5c5ccccc45)cc3)n2)nc1 ; c1cnc2c(c1)ccc1ccc(-c3ccc(-c4ccc(-c5nc6c7ccccc7c7ccccc7c6c6ccccc56)cc4)cc3)nc12]

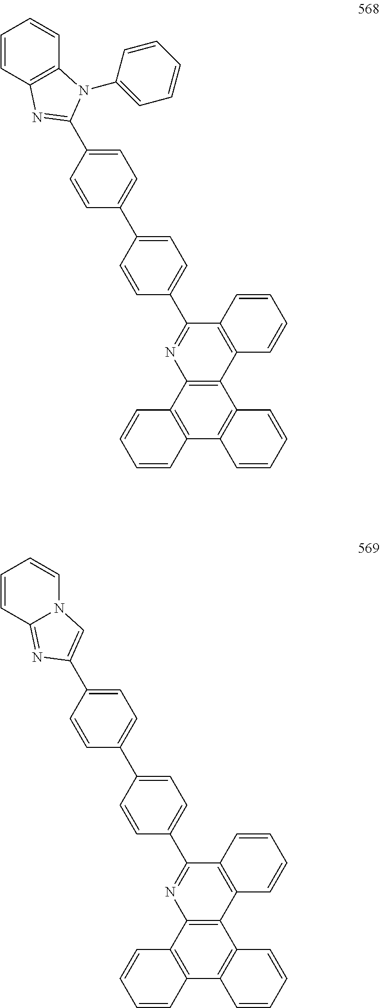 [SMILES: c1ccc(-n2c(-c3ccc(-c4ccc(-c5nc6c7ccccc7c7ccccc7c6c6ccccc56)cc4)cc3)nc3ccccc32)cc1 ; c1ccc2c(c1)c(-c1ccc(-c3ccc(-c4cn5ccccc5n4)cc3)cc1)nc1c3ccccc3c3ccccc3c21]

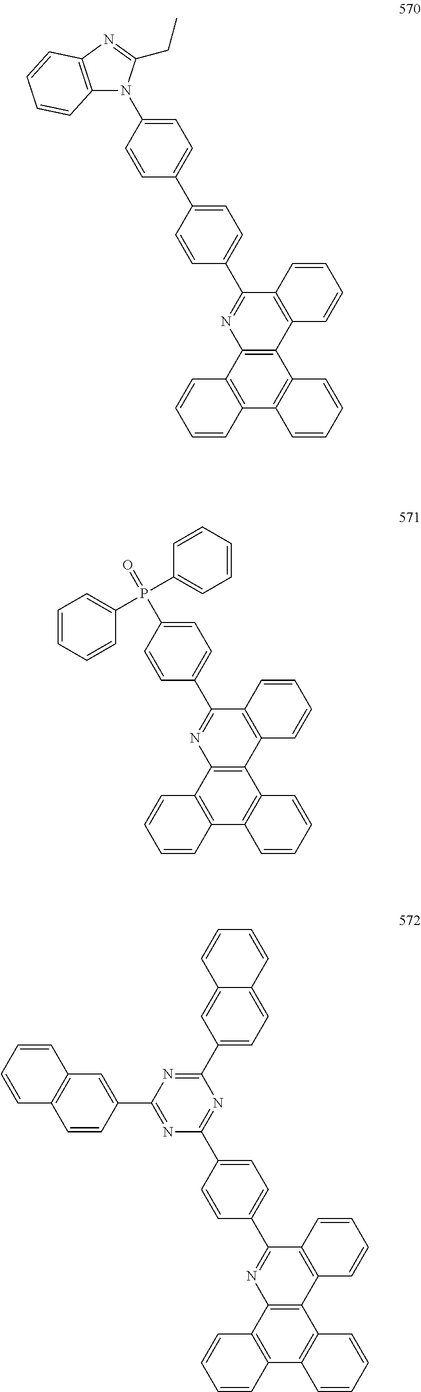 [SMILES: CCc1nc2ccccc2n1-c1ccc(-c2ccc(-c3nc4c5ccccc5c5ccccc5c4c4ccccc34)cc2)cc1 ; O=P(c1ccccc1)(c1ccccc1)c1ccc(-c2nc3c4ccccc4c4ccccc4c3c3ccccc23)cc1 ; c1ccc2cc(-c3nc(-c4ccc(-c5nc6c7ccccc7c7ccccc7c6c6ccccc56)cc4)nc(-c4ccc5ccccc5c4)n3)ccc2c1]

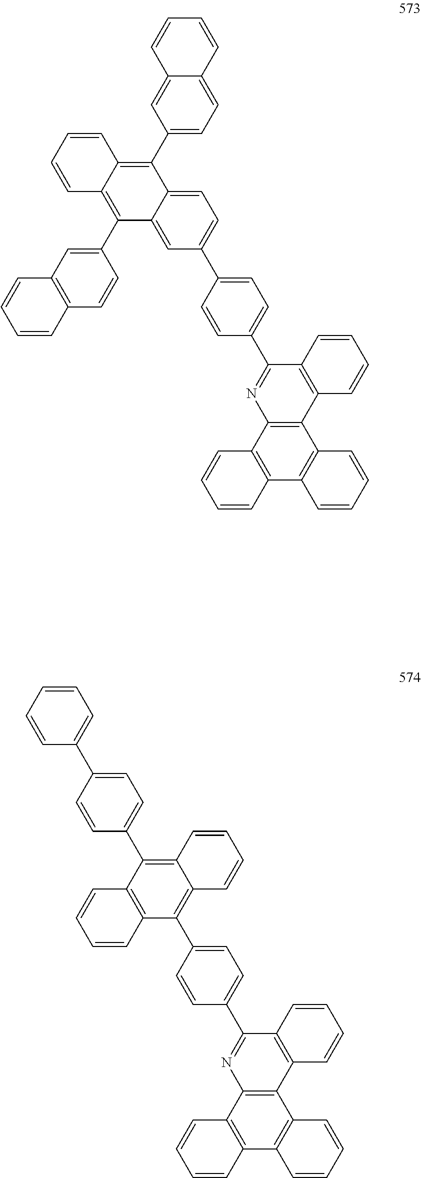 [SMILES: c1ccc2cc(-c3c4ccccc4c(-c4ccc5ccccc5c4)c4cc(-c5ccc(-c6nc7c8ccccc8c8ccccc8c7c7ccccc67)cc5)ccc34)ccc2c1 ; c1ccc(-c2ccc(-c3c4ccccc4c(-c4ccc(-c5nc6c7ccccc7c7ccccc7c6c6ccccc56)cc4)c4ccccc34)cc2)cc1]

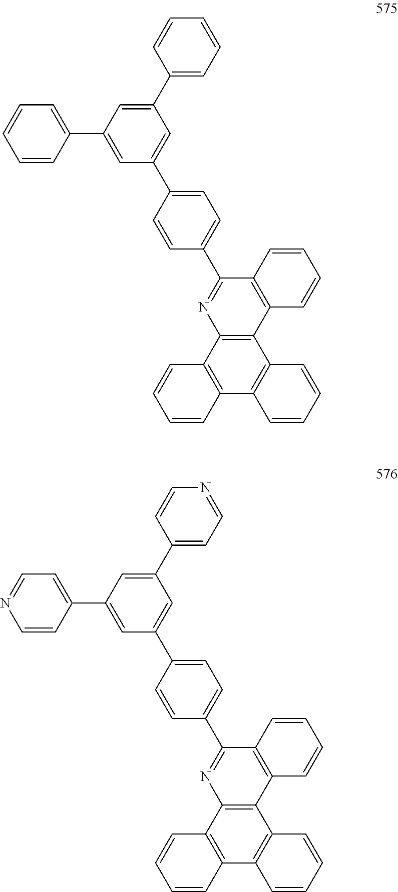 [SMILES: c1ccc(-c2cc(-c3ccccc3)cc(-c3ccc(-c4nc5c6ccccc6c6ccccc6c5c5ccccc45)cc3)c2)cc1 ; c1ccc2c(c1)c(-c1ccc(-c3cc(-c4ccncc4)cc(-c4ccncc4)c3)cc1)nc1c3ccccc3c3ccccc3c21]

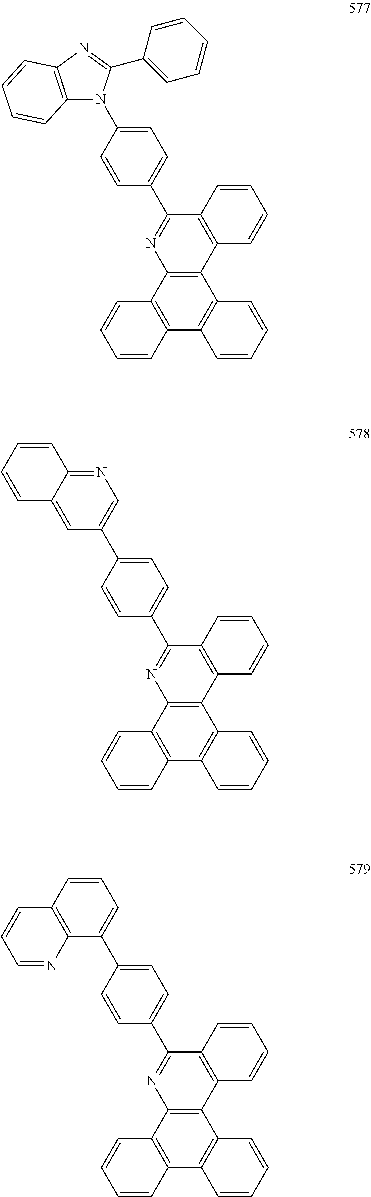 [SMILES: c1ccc(-c2nc3ccccc3n2-c2ccc(-c3nc4c5ccccc5c5ccccc5c4c4ccccc34)cc2)cc1 ; c1ccc2ncc(-c3ccc(-c4nc5c6ccccc6c6ccccc6c5c5ccccc45)cc3)cc2c1 ; c1cnc2c(-c3ccc(-c4nc5c6ccccc6c6ccccc6c5c5ccccc45)cc3)cccc2c1]

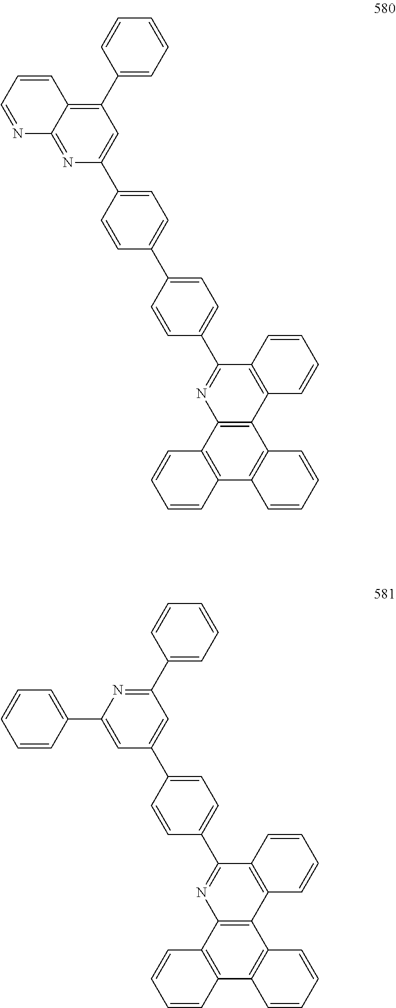 [SMILES: c1ccc(-c2cc(-c3ccc(-c4ccc(-c5nc6c7ccccc7c7ccccc7c6c6ccccc56)cc4)cc3)nc3ncccc23)cc1 ; c1ccc(-c2cc(-c3ccc(-c4nc5c6ccccc6c6ccccc6c5c5ccccc45)cc3)cc(-c3ccccc3)n2)cc1]

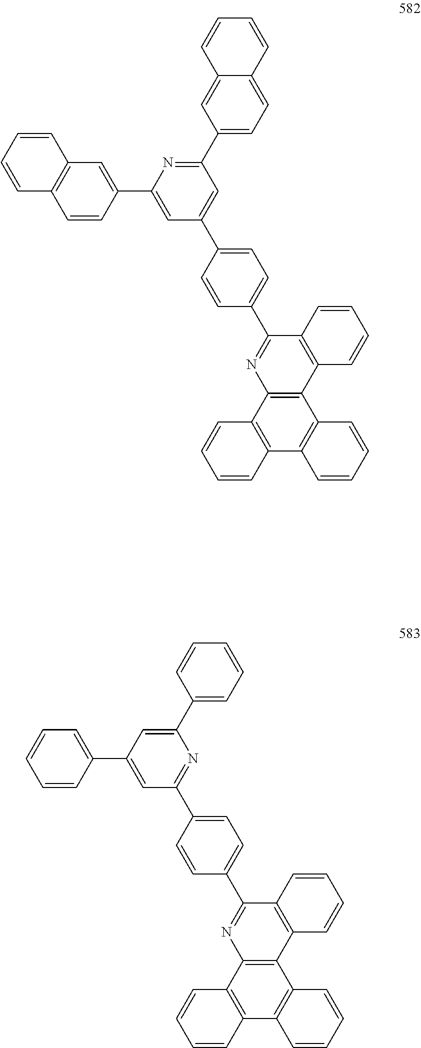 [SMILES: c1ccc2cc(-c3cc(-c4ccc(-c5nc6c7ccccc7c7ccccc7c6c6ccccc56)cc4)cc(-c4ccc5ccccc5c4)n3)ccc2c1 ; c1ccc(-c2cc(-c3ccccc3)nc(-c3ccc(-c4nc5c6ccccc6c6ccccc6c5c5ccccc45)cc3)c2)cc1]

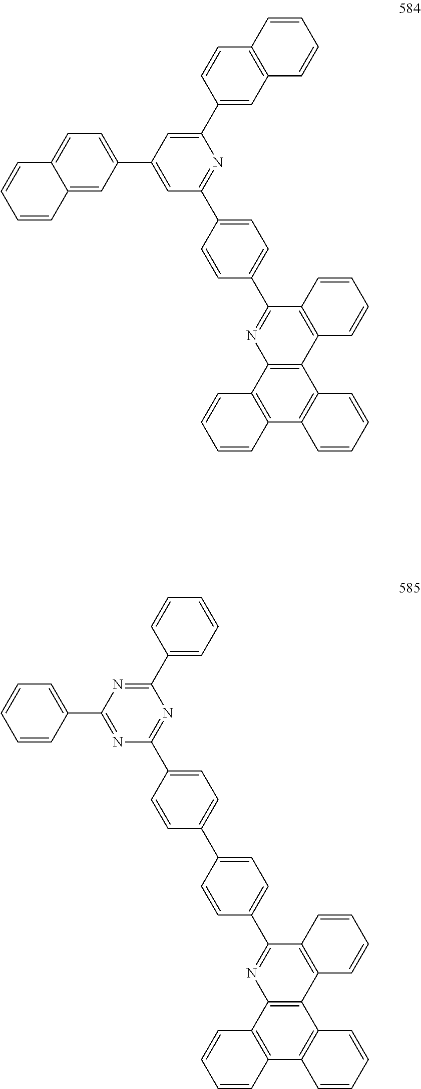 [SMILES: c1ccc2cc(-c3cc(-c4ccc(-c5nc6c7ccccc7c7ccccc7c6c6ccccc56)cc4)nc(-c4ccc5ccccc5c4)c3)ccc2c1 ; c1ccc(-c2nc(-c3ccccc3)nc(-c3ccc(-c4ccc(-c5nc6c7ccccc7c7ccccc7c6c6ccccc56)cc4)cc3)n2)cc1]

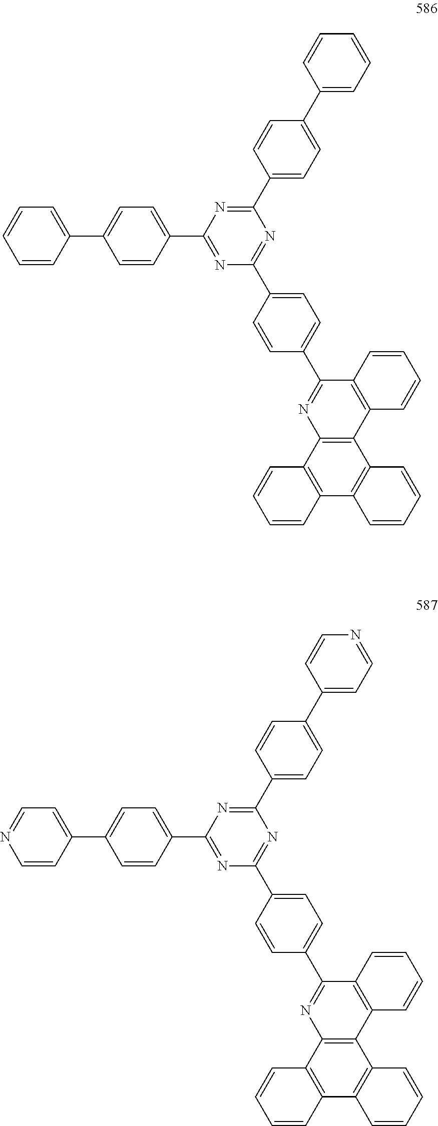 [SMILES: c1ccc(-c2ccc(-c3nc(-c4ccc(-c5ccccc5)cc4)nc(-c4ccc(-c5nc6c7ccccc7c7ccccc7c6c6ccccc56)cc4)n3)cc2)cc1 ; c1ccc2c(c1)c(-c1ccc(-c3nc(-c4ccc(-c5ccncc5)cc4)nc(-c4ccc(-c5ccncc5)cc4)n3)cc1)nc1c3ccccc3c3ccccc3c21]

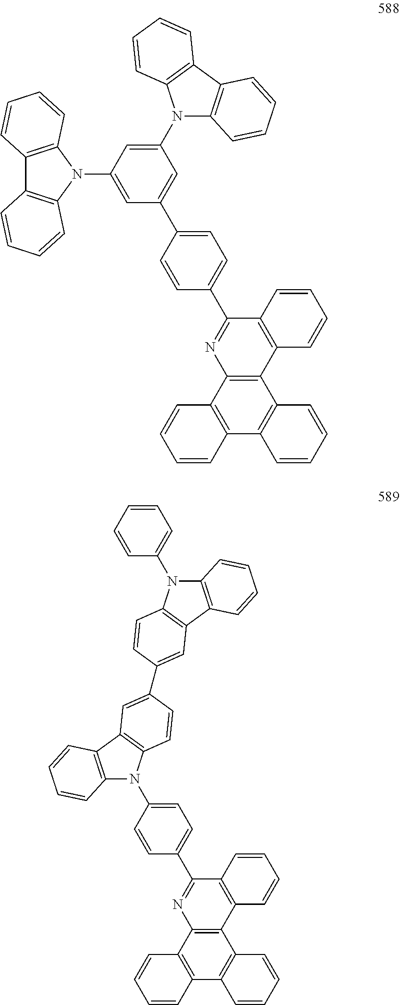 [SMILES: c1ccc2c(c1)c(-c1ccc(-c3cc(-n4c5ccccc5c5ccccc54)cc(-n4c5ccccc5c5ccccc54)c3)cc1)nc1c3ccccc3c3ccccc3c21 ; c1ccc(-n2c3ccccc3c3cc(-c4ccc5c(c4)c4ccccc4n5-c4ccc(-c5nc6c7ccccc7c7ccccc7c6c6ccccc56)cc4)ccc32)cc1]

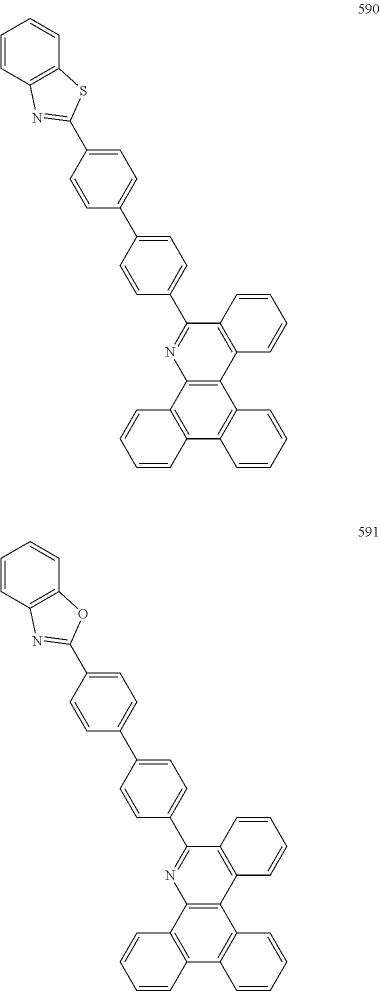 [SMILES: c1ccc2sc(-c3ccc(-c4ccc(-c5nc6c7ccccc7c7ccccc7c6c6ccccc56)cc4)cc3)nc2c1 ; c1ccc2oc(-c3ccc(-c4ccc(-c5nc6c7ccccc7c7ccccc7c6c6ccccc56)cc4)cc3)nc2c1]

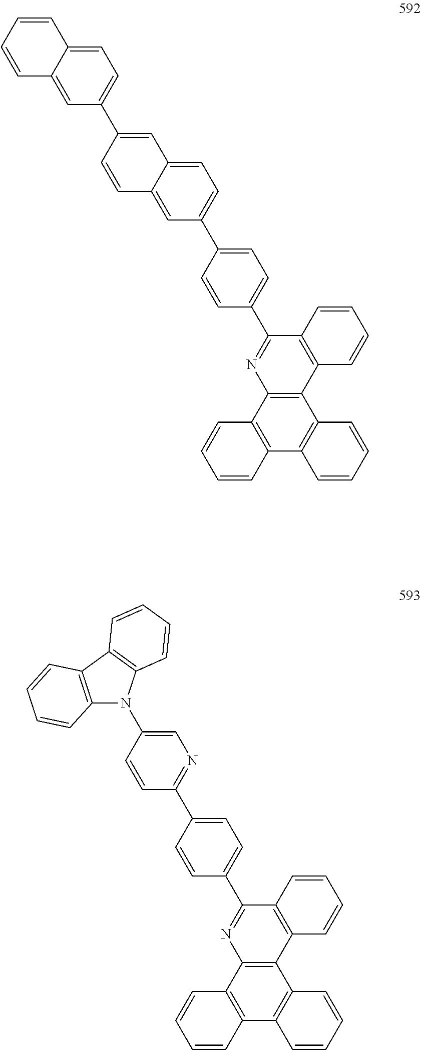 [SMILES: c1ccc2cc(-c3ccc4cc(-c5ccc(-c6nc7c8ccccc8c8ccccc8c7c7ccccc67)cc5)ccc4c3)ccc2c1 ; c1ccc2c(c1)c(-c1ccc(-c3ccc(-n4c5ccccc5c5ccccc54)cn3)cc1)nc1c3ccccc3c3ccccc3c21]

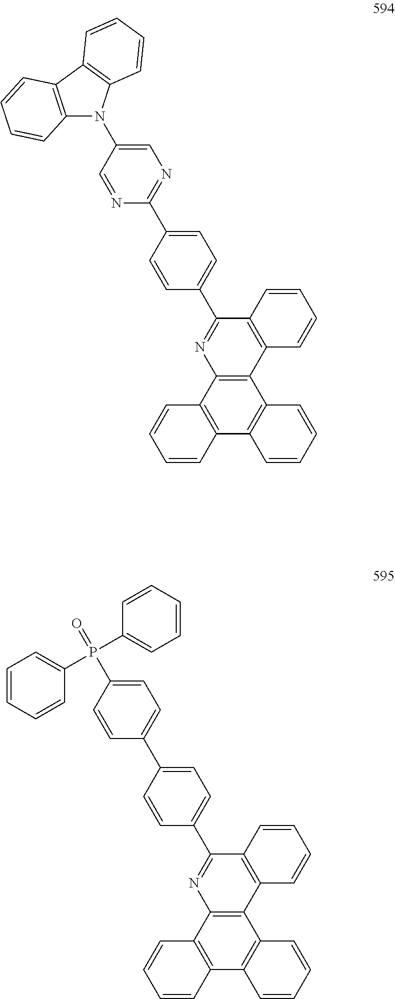 [SMILES: c1ccc2c(c1)c(-c1ccc(-c3ncc(-n4c5ccccc5c5ccccc54)cn3)cc1)nc1c3ccccc3c3ccccc3c21 ; O=P(c1ccccc1)(c1ccccc1)c1ccc(-c2ccc(-c3nc4c5ccccc5c5ccccc5c4c4ccccc34)cc2)cc1]

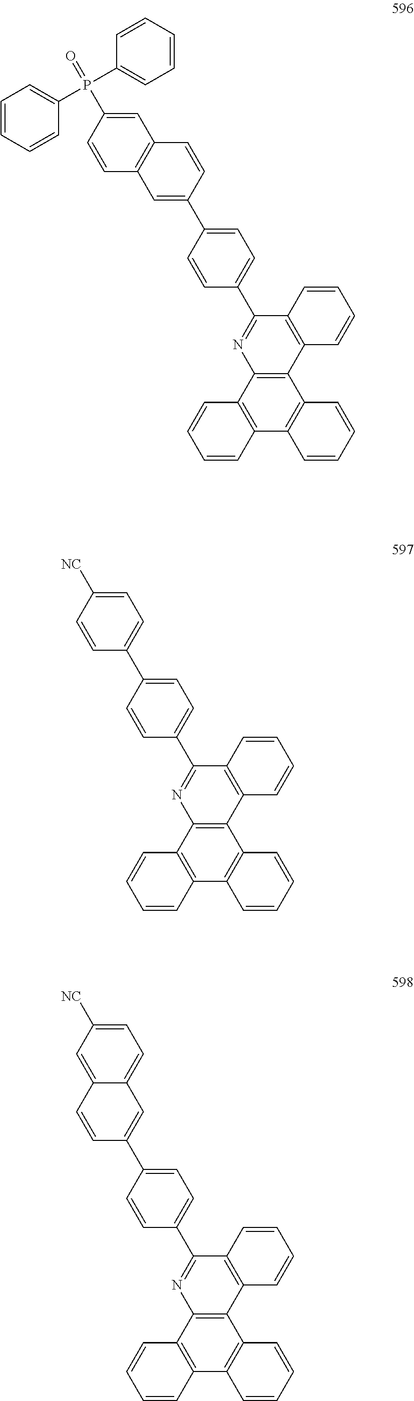 [SMILES: O=P(c1ccccc1)(c1ccccc1)c1ccc2cc(-c3ccc(-c4nc5c6ccccc6c6ccccc6c5c5ccccc45)cc3)ccc2c1 ; N#Cc1ccc(-c2ccc(-c3nc4c5ccccc5c5ccccc5c4c4ccccc34)cc2)cc1 ; N#Cc1ccc2cc(-c3ccc(-c4nc5c6ccccc6c6ccccc6c5c5ccccc45)cc3)ccc2c1]

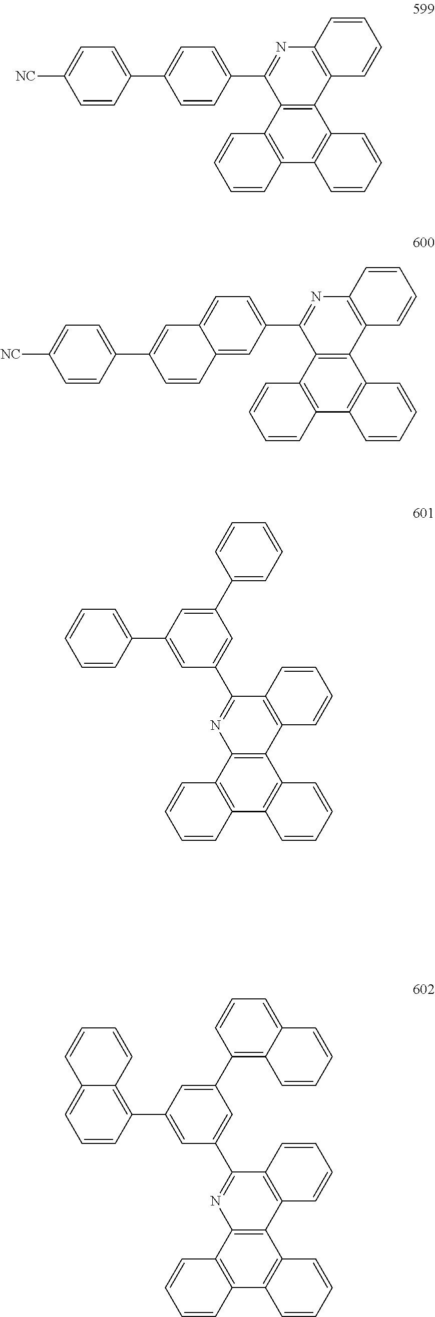 [SMILES: N#Cc1ccc(-c2ccc(-c3nc4ccccc4c4c5ccccc5c5ccccc5c34)cc2)cc1 ; N#Cc1ccc(-c2ccc3cc(-c4nc5ccccc5c5c6ccccc6c6ccccc6c45)ccc3c2)cc1 ; c1ccc(-c2cc(-c3ccccc3)cc(-c3nc4c5ccccc5c5ccccc5c4c4ccccc34)c2)cc1 ; c1ccc2c(-c3cc(-c4cccc5ccccc45)cc(-c4nc5c6ccccc6c6ccccc6c5c5ccccc45)c3)cccc2c1]

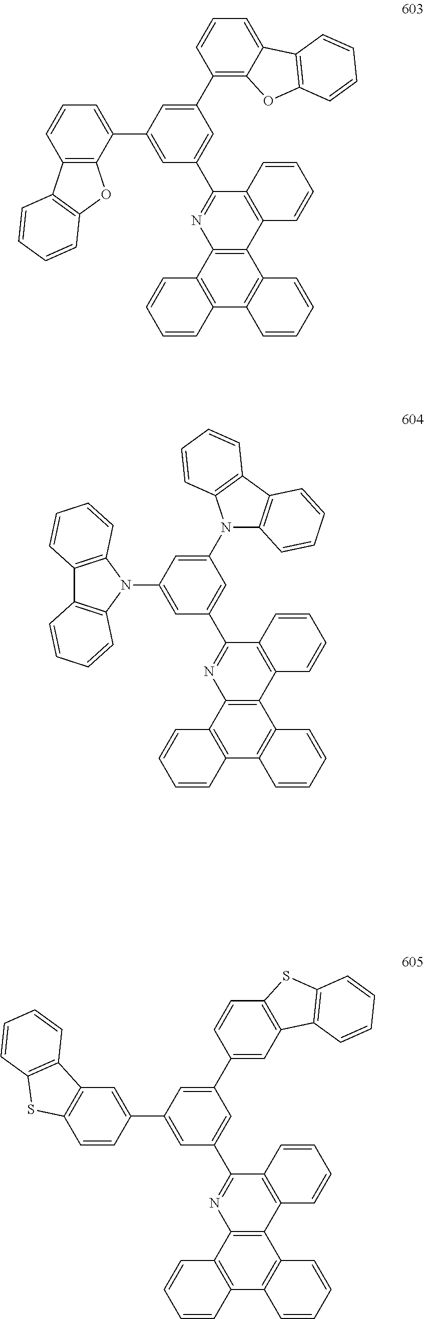 [SMILES: c1ccc2c(c1)oc1c(-c3cc(-c4nc5c6ccccc6c6ccccc6c5c5ccccc45)cc(-c4cccc5c4oc4ccccc45)c3)cccc12 ; c1ccc2c(c1)c(-c1cc(-n3c4ccccc4c4ccccc43)cc(-n3c4ccccc4c4ccccc43)c1)nc1c3ccccc3c3ccccc3c21 ; c1ccc2c(c1)sc1ccc(-c3cc(-c4ccc5sc6ccccc6c5c4)cc(-c4nc5c6ccccc6c6ccccc6c5c5ccccc45)c3)cc12]

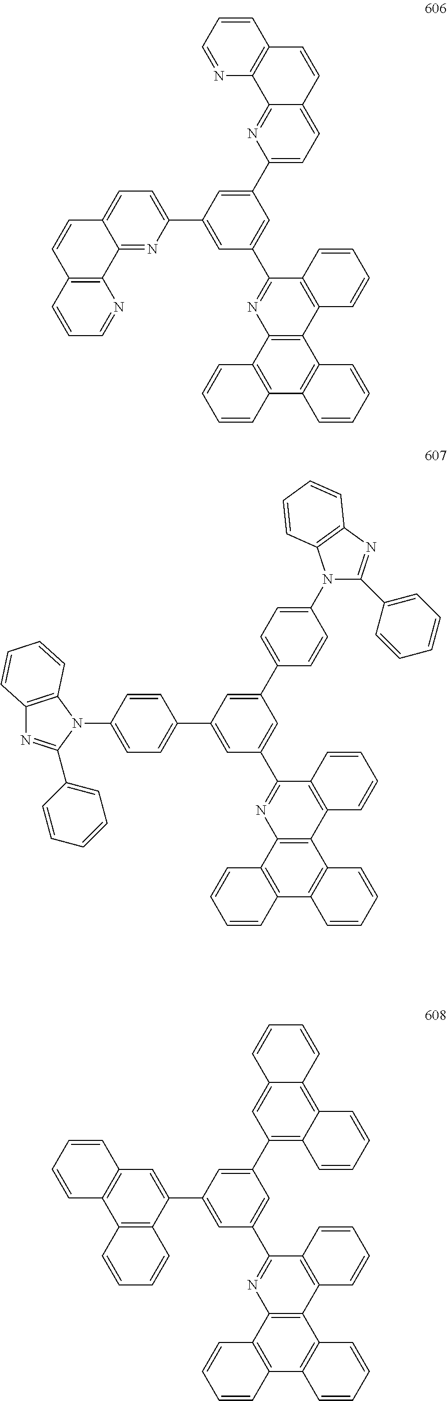 [SMILES: c1cnc2c(c1)ccc1ccc(-c3cc(-c4ccc5ccc6cccnc6c5n4)cc(-c4nc5c6ccccc6c6ccccc6c5c5ccccc45)c3)nc12 ; c1ccc(-c2nc3ccccc3n2-c2ccc(-c3cc(-c4ccc(-n5c(-c6ccccc6)nc6ccccc65)cc4)cc(-c4nc5c6ccccc6c6ccccc6c5c5ccccc45)c3)cc2)cc1 ; c1ccc2c(c1)cc(-c1cc(-c3cc4ccccc4c4ccccc34)cc(-c3nc4c5ccccc5c5ccccc5c4c4ccccc34)c1)c1ccccc12]

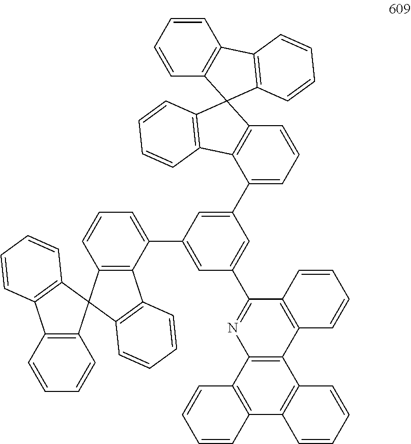 [SMILES: c1ccc2c(c1)-c1ccccc1C21c2ccccc2-c2c(-c3cc(-c4cccc5c4-c4ccccc4C54c5ccccc5-c5ccccc54)cc(-c4nc5c6ccccc6c6ccccc6c5c5ccccc45)c3)cccc21]

The compounds described above may be prepared based on the preparation examples described below. The following preparation examples are representative examples, but if necessary, substituents may be added or excluded and positions of substituents may be changed. Further, based on technologies known in the art, starting materials, reactants, reaction conditions, and the like may be changed. If necessary, kinds or positions of the substituents at the other positions may be modified by those skilled in the art using technologies known in the art.

For example, the compound of the chemical formula 2 may be manufactured into a core structure of the following general formula 1.

A substituent can be bonded by a method known in the art, and a position of a substituent or the number of substitutents can be modified according to technologies known in the art.

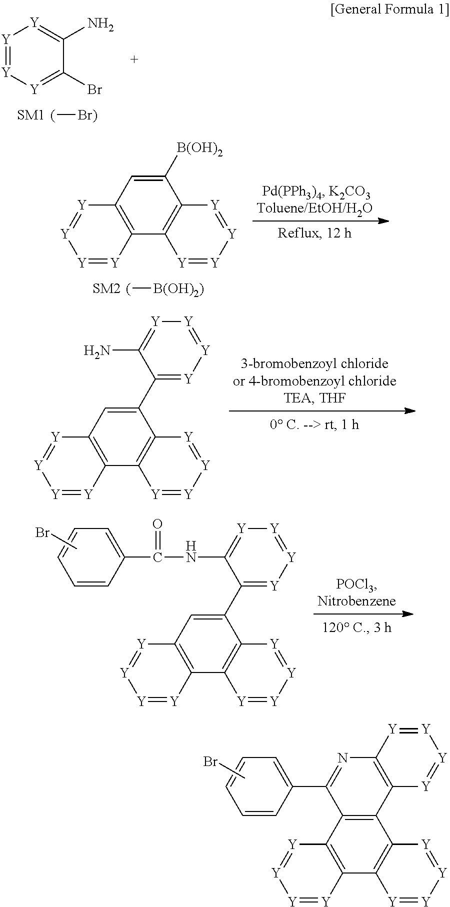

In the general formula 1, Y₁ to Y₁₂ are the same as defined in the chemical formula 1.

The general formula 1 is an example of an intermediate reaction for bonding a substituent at a position of R₁ in a core structure of the chemical formula 2. To be specific, the final compound of the general formula 1 is phenyl of which R₁ is substituted with Br in the chemical formula 2. The Br may be changed to another substituent as necessary, and the phenyl can also be changed to another substituent by changing benzoyl chloride as a reactant.

For example, the compound of the chemical formula 3 may be manufactured into a core structure of the following general formula 2.

A substituent can be bonded by a method known in the art, and a position of a substituent or the number of substitutents can be modified according to technologies known in the art.

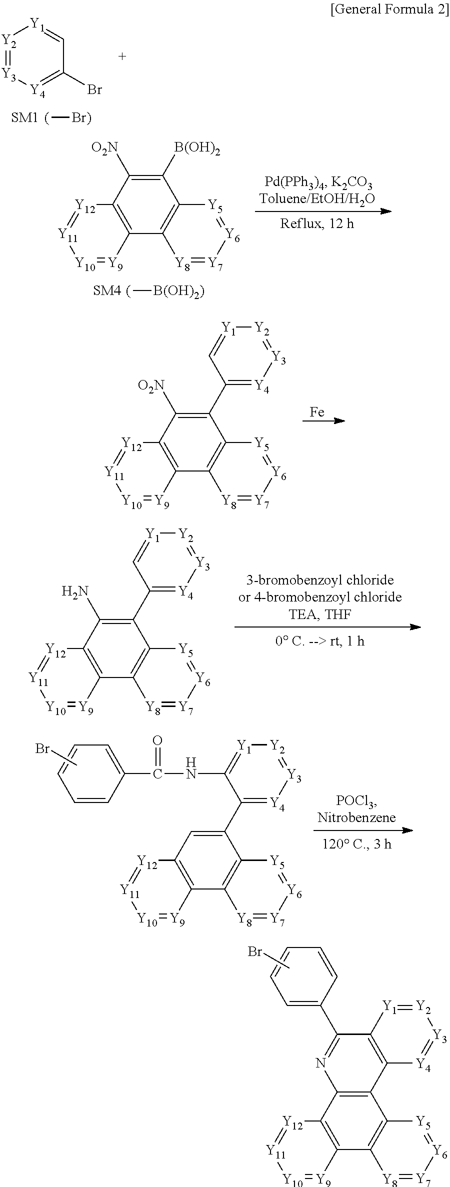

In the general formula 2, Y₁ to Y₁₂ are the same as defined in the chemical formula 1.

The general formula 2 is an example of an intermediate reaction for bonding a substituent at a position of R₁ in a core structure of the chemical formula 3. To be specific, the final compound of the general formula 2 is phenyl of which R₁ is substituted with Br in the chemical formula 3. The Br may be changed to another substituent as necessary, and the phenyl can also be changed to another substituent by changing benzoyl chloride as a reactant.

Another exemplary embodiment of the present invention provides an organic light emitting device including the compound of the chemical formula 1. To be specific, the organic light emitting device includes an anode, a cathode, and one or more organic material layers provided between the anode and the cathode, wherein one or more layers of the organic material layers include the compound of the chemical formula 1.

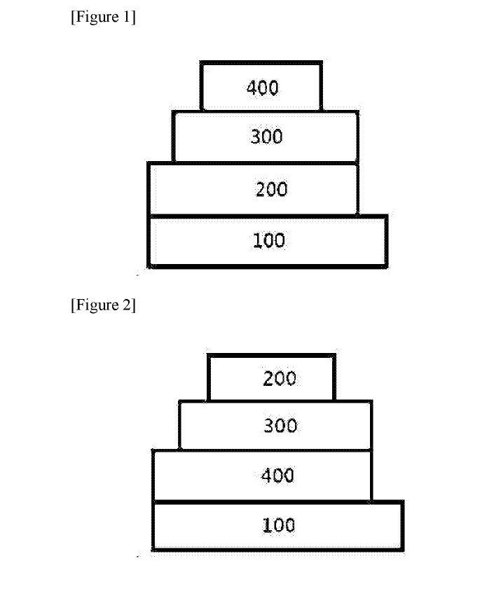

FIGS. 1 to 3 illustrate examples of laminating order of electrodes and organic material layers of an organic light emitting device according to exemplary embodiments of the present invention. However, these drawings are not provided for limiting the scope of the present invention, and the structure of the organic light emitting device known in the art can also be applied to the present invention.

Referring to FIG. 1, an organic light emitting device in which an anode 200, an organic material layer 300, and a cathode 400 are laminated in sequence on a substrate 100 is illustrated by the diagram. However, the structure of the organic light emitting device is not limited to this structure only, and as illustrated in FIG. 2, an organic light emitting device in which a cathode, an organic material layer, and an anode are laminated in sequence on a substrate may also be included.

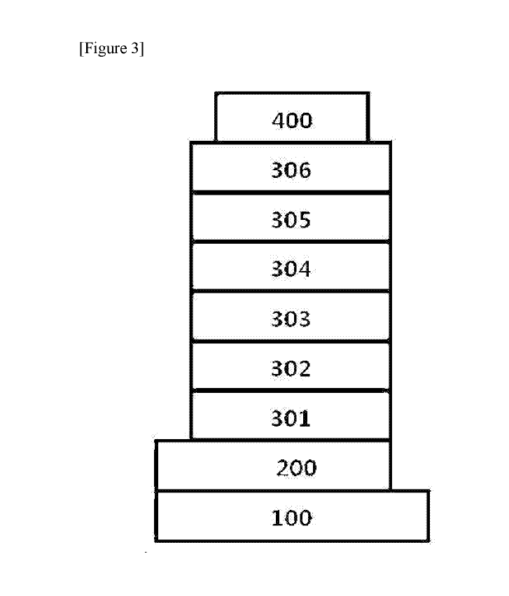

FIG. 3 illustrates the case where the organic material layer is a multilayer. An organic light emitting device illustrated in FIG. 3 includes a hole injection layer 301, a hole transport layer 302, a light emitting layer 303, a hole blocking layer 304, an electron transport layer 305, and an electron injection layer 306. However; the scope of the present invention is not limited to this laminated structure, and when necessary, other layers except the light emitting layer may not be included, and other necessary layers having other functions may be added.

An organic light emitting device according to the present invention may be prepared using materials and methods known in the art except that the compound of the chemical formula 1 is included in one or more layers of the organic material layers.

The compound of the chemical formula 1 may form one or more layers of the organic material layers alone in an organic light emitting device. However, when necessary, the compound of the chemical formula 1 may be mixed with other materials to form the organic material layers.

The compound of the chemical formula 1 may be used as a hole injection material, a hole transport material, a light emitting material, a hole blocking material, an electron transport material, an electron injection material, or the like, in an organic light emitting device.

For example, the compound according to the exemplary embodiment of the present invention may be used as a material of an electron injection layer, an electron transport layer, or a layer that simultaneously injects and transports an electron in an organic light emitting device.

Further, the compound according to the exemplary embodiment of the present invention may be used as a material of a light emitting layer in an organic light emitting device. To be specific, the compound may be used alone as a light emitting material, or may be used as a host material or a dopant material of a light emitting layer.

Furthermore, the compound according to the exemplary embodiment of the present invention may be used as a phosphorescent host material in an organic light emitting device. In this case, the compound according to an exemplary embodiment of the present invention is included together with a phosphorescent dopant.

Moreover, the compound according to the exemplary embodiment of the present invention may be used as a material of a hole blocking layer in an organic light emitting device.

In the organic light emitting device according to the present invention, other materials than the compound of the chemical formula 1 are illustrated below, but they are for illustrative purposes only, and are not intended to limit the scope of the present invention, and can be substituted with materials known in the art.

As the anode material, materials having relatively large work function may be used, and transparent conductive oxides, metals, conductive polymers, or the like may be used.

As the cathode material, materials having relatively small work function may be used, and metals, metal oxides, conductive polymers, or the like may be used.

As the hole injection material, hole injection materials known in the art may be used, and for example, phthalocyanine compounds such as copper phthalocyanine disclosed in U.S. Pat. No. 4,356,429, or starbust-type amine derivatives disclosed in a literature [Advanced Material, 6, p. 677 (1994)], such as TCTA, m-MTDATA, m-MTDAPB, Pani/DBSA (polyaniline/dodecylbenzenesulfonic acid) or PEDOT/PSS (poly(3,4-ethylenedioxythiophene)/poly(4-styrenesulfonate)), Pani/CSA (polyaniline/camphor sulfonic acid) or PANI/PSS (polyaniline/poly(4-styrene-sulfonate), which is a conductive polymer having solubility, or the like, may be used.

As the hole transport material, a pyrazoline derivative, an arylamine-based derivative, a stilbene derivative, a triphenyldiamine derivative, or the like may be used, and a low molecular or high molecular material may also be used.

As the electron transport material, an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, 8-hydroxyquinoline and a metal complex of a derivative thereof, or the like, may be used, and a high molecular material as well as a low molecular material may also be used.

As the electron injection material, for example, LiF is typically used in the related industry. However, the present invention is not limited thereto.

As the light emitting material, a red, green, or blue light emitting material may be used, and when necessary, two or more light emitting materials may be mixed and used. Further, as the light emitting material, a fluorescent material may be used and a phosphorescent material may also be used. As the light emitting material, materials that emit light alone by bonding the holes and the electrons injected from an anode and a cathode, respectively, may be used. However, materials in which a host material and a dopant material are both involved in light emitting may also be used.

If the compound according to the present invention is used as a phosphorescent host material, a phosphorescent dopant material to be used together may employ those known in the art.

For example, phosphorescent dopant materials as expressed by LL′MX, LL′L″M, LMXX′, L₂MX, and L₃M may be used, but the present invention is not limited thereto.

Herein, L, L′, L″, X, and X′ are not equivalent, bidentate ligands, and M is a metal that forms octahedral complexes.

M may be iridium, platinum, osmium, or the like.

L is an anionic bidentate ligand which coordinates to M via an sp² hybridized carbon and a heteroatom, and X functions to trap electrons or holes. Non-limiting examples of the L may include 2-(1-naphthyl)benzoxazole, (2-phenylbenzoxazole), (2-phenylbenzothiazole), (2-phenylbenzothiazole), (7,8-benzoquinoline), (thienylpyridine), phenylpyridine, benzothienylpyridine, 3-methoxy-2-phenylpyridine, thienylpyridine, and tolylpyridine. Non-limiting examples of the X may include acetylacetonate (acac), hexafluoroacetylacetonate, salicylidene, picolinate, and 8-hydroxyquinolinate.

More specific examples will be described below, but the present invention is not limited thereto.

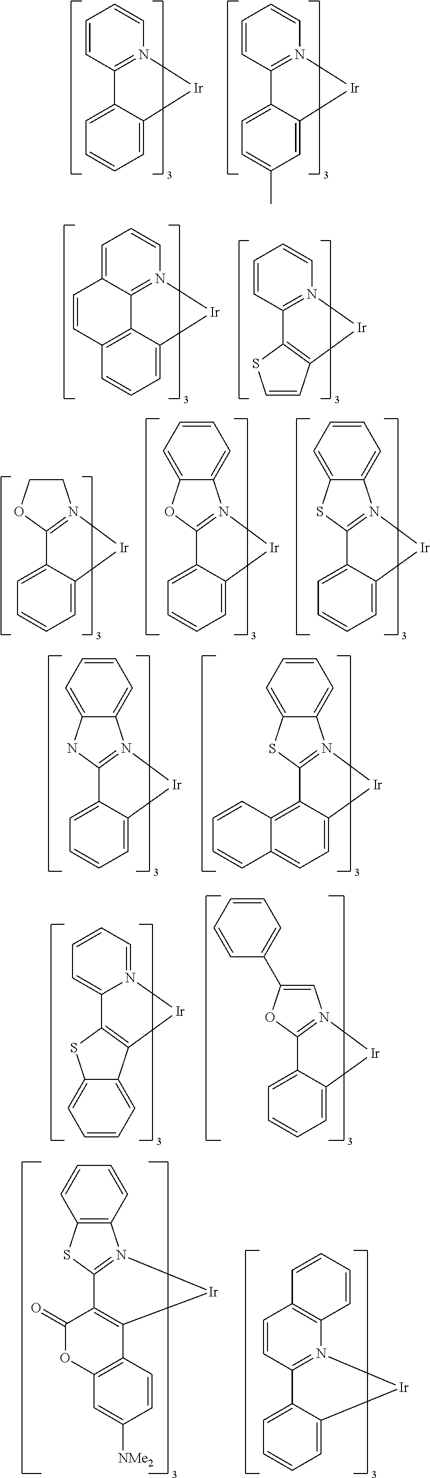

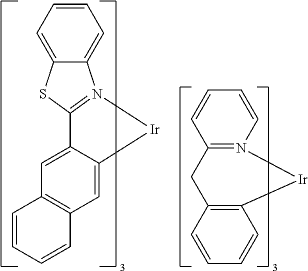

Hereinafter, the present invention will be described in more detail with reference to examples, however, it is to be understood that these are for illustrative purposes only, and are not intended to limit the scope of the present invention.

### Preparation Example 1

### Preparation of Compound 2

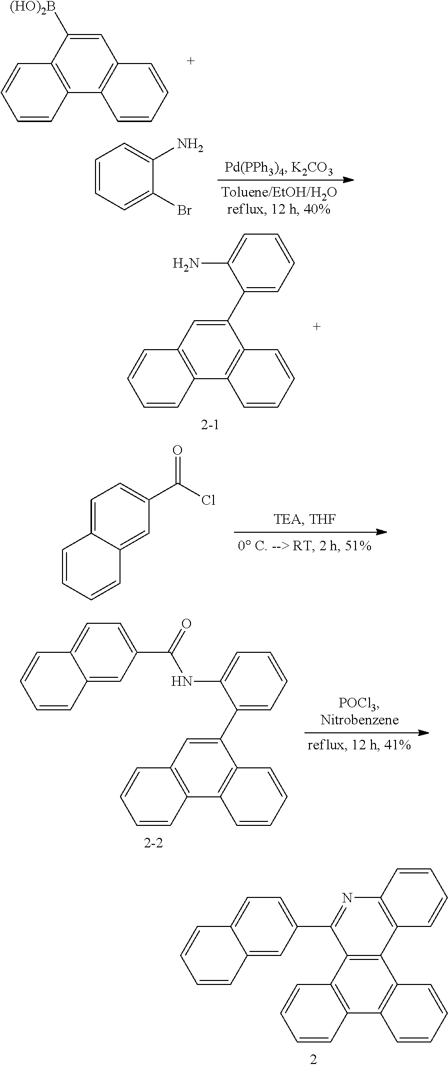

Preparation of Compound 2-1

A mixed solution of 50.6 g (0.294 mol) of 2-bromoaniline, 71.8 g (0.324 mol) of phenanthren-9-yl boronic acid, K₂CO₃ (121.7 g, 0.882 mmol), Pd(PPh₃)₄ (17.0 g, 0.0147 mol), and toluene, EtOH, H₂O (500 ml/200 ml/100 ml) was put into a two neck round bottom flask (two neck r.b.f) and refluxed and stirred therein under nitrogen for 12 hours. A reaction mixture cooled to room temperature was extracted with MC (methylene chloride)/H₂O and dried using MgSO₄ and then filtered. After being concentrated, the resultant reaction product was separated by column chromatography (SiO₂, hexane:MC (methylene chloride)=2:1). As a result, an ivory solid compound 2-1 (32 g, 40%) was obtained.

Preparation of Compound 2-2

Compound 2-1 (10.0 g, 0.0371 mol) was dissolved in 100 ml of THF (tetrahydrofuran) within a one neck round bottom flask (one neck r.b.f) under nitrogen, and triethylamine (15.4 ml, 0.111 mol) was added dropwise thereto at room temperature. After the reaction mixture was cooled to 0° C., 2-naphthoyl chloride was dissolved in 50 ml of THF and slowly added dropwise thereto at a low temperature. Then, the resultant reaction product was stirred at room temperature for 2 hours and added with H₂O to terminate the reaction. The solvent was distilled under reduced pressure, and then MC (methylene chloride)/H₂O was extracted. The resultant reaction product was dried using MgSO₄ and then filtered. After distillation under reduced pressure and concentration, the resultant reaction product was dissolved in a small amount of MC (methylene chloride) and added with methanol to precipitate a solid. As a result, an ivory solid compound 2-2 (8.01 g, 51%) was obtained.

Preparation of Compound 2

Compound 2-2 (8.01 g, 0.0189 mol) was dissolved in nitrobenzene (80 ml) within a one neck round bottom flask (one neck r.b.f), and POCl₃ (phosphorous oxychloride) (0.35 ml, 3.78 mmol) was added dropwise thereto at room temperature and then refluxed and stirred for 12 hours. After the reaction was terminated, the mixed solution was cooled to room temperature and neutralized with NaHCO₃ aqueous solution. Then, MC (methylene chloride)/H₂O was extracted. The resultant reaction product was dried using MgSO₄ and then filtered. After the solvent was distilled under reduced pressure and concentrated, a small amount of MC (methylene chloride) and an excessive amount of hexane were added to precipitate a solid. As a result, a yellow solid compound 2 (4.67 g, 61%) was obtained.

### Preparation Example 2

### Preparation of Compound 18

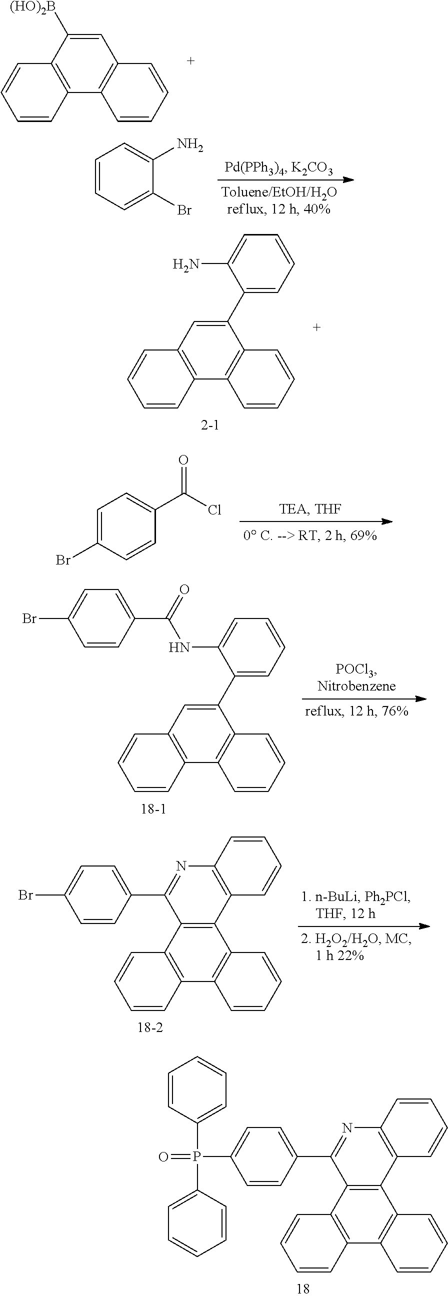

Preparation of Compound 18-1

Compound 2-1 (32 g, 0.119 mol) was dissolved in THF (200 ml) within a one neck round bottom flask (one neck r.b.f) under nitrogen, and triethylamine (49.4 ml, 0.356 mol) was added dropwise thereto at room temperature. After the reaction mixture was cooled to 0° C., 39.2 g (0.179 mol) of 4-bromobenzoyl chloride was dissolved in 50 ml of THF and slowly added dropwise thereto at a low temperature. Then, the resultant reaction product was stirred at room temperature for 2 hours and added with H₂O to terminate the reaction. The solvent was distilled under reduced pressure, and then MC (methylene chloride)/H₂O was extracted. The resultant reaction product was dried using MgSO₄ and then filtered. After distillation under reduced pressure and concentration, the resultant reaction product was dissolved in a small amount of MC (methylene chloride) and added with methanol to precipitate a solid. As a result, an ivory solid compound 18-1 (37.1 g, 69%) was obtained.

Preparation of Compound 18-2

Compound 18-1 (20 g, 0.0442 mol) was dissolved in nitrobenzene (150 ml) within a one neck round bottom flask (one neck r.b.f), and POCl₃ (phosphorous oxychloride) (0.82 ml, 8.84 mmol) was added dropwise thereto at room temperature and then refluxed and stirred for 12 hours. After the reaction was terminated, the mixed solution was cooled to room temperature and neutralized with NaHCO₃ aqueous solution. Then, MC (methylene chloride)/H₂O was extracted. The resultant reaction product was dried using MgSO₄ and then filtered. After the solvent was distilled under reduced pressure and concentrated, a small amount of MC (methylene chloride) and an excessive amount of hexane were added to precipitate a solid. As a result, a pale yellow solid compound 18-2 (14.5 g, 76%) was obtained.

Preparation of Compound 18

Compound 18-2 (10 g, 0.023 mol) was dissolved in anhydrous THF (50 ml) within a one neck round bottom flask (one neck r.b.f) under nitrogen and then cooled to −78° C. Then, n-butyllithium (2.5 M in hexane) (12 ml, 0.0299 mol) was slowly added dropwise thereto and stirred for 1 hour. 5.37 ml (0.0299 mol) of chlorodiphenylphosphine was added dropwise to the above solution and stirred at room temperature for 12 hours. The reaction mixture was extracted with MC (methylene chloride)/H₂O and distilled under reduced pressure. The reaction mixture was dissolved in 250 ml of MC (methylene chloride) and then stirred with 20 ml of 30% H₂O₂ aqueous solution at room temperature for 1 hour. The reaction mixture was extracted with MC (methylene chloride)/H₂O, and then the concentrated mixture was separated by column chromatography (SiO₂, MC (methylene chloride):methanol=25:1). As a result, a yellow solid compound 18 (2.81 g, 22%) was obtained.

### Preparation Example 3

### Preparation of Compound 22

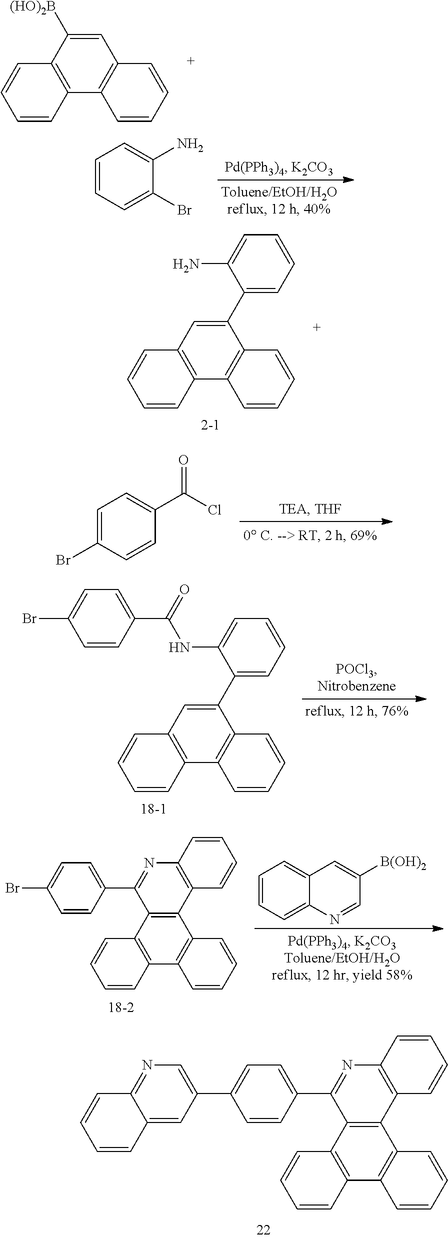

Preparation of Compound 22

A mixed solution of Compound 18-2 (10.0 g, 0.023 mol), 4.38 g (0.0253 mol) of quinolin-3-yl boronic acid, K₂CO₃ (9.52 g, 0.069 mmol), Pd(PPh₃)₄ (1.33 g, 1.15 mmol), and toluene, EtOH, H₂O (200 ml/30 ml/30 ml) was put into a two neck round bottom flask (two neck r.b.f) and refluxed and stirred therein under nitrogen for 12 hours. A reaction mixture cooled to room temperature was extracted with MC (methylene chloride)/H₂O and dried using MgSO₄ and then filtered. After being concentrated, the resultant reaction product was separated by column chromatography (SiO₂, hexane:ethyl acetate=2:1). As a result, a yellow solid compound 22 (6.4 g, 58%) was obtained.

### Preparation Example 4

### Preparation of Compound 37

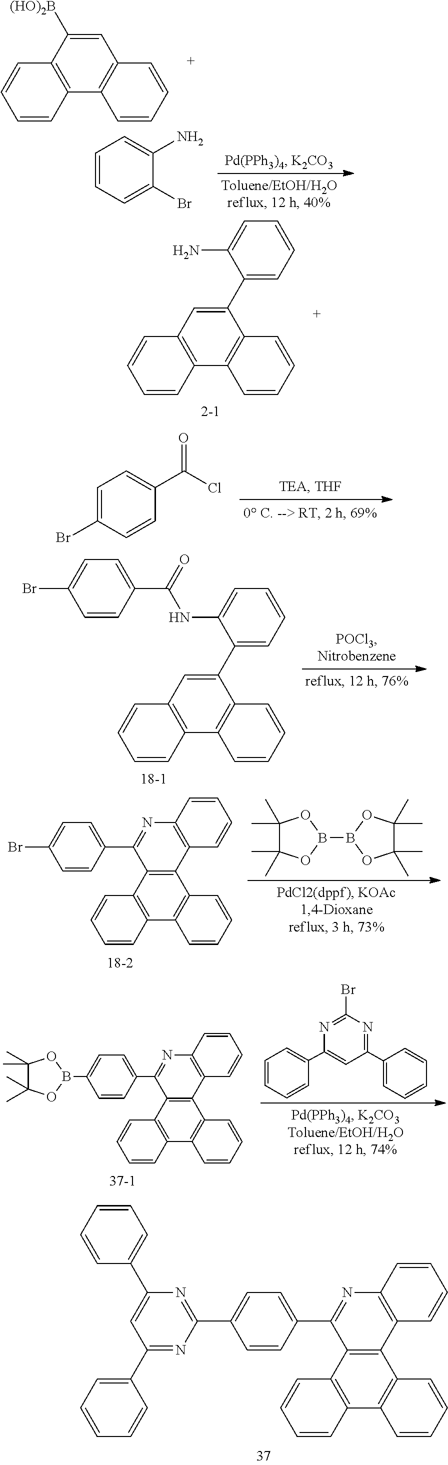

Preparation of Compound 37-1

A mixed solution of Compound 18-2 (13.6 g, 0.0313 mol), 10.3 g (0.0407 mol) of bis(pinacolato)diboron, 9.21 g (0.0939 mol) of KOAc (potassium acetate), 1.14 g (1.56 mmol) of PdCl₂(dppf), and 250 ml of 1,4-dioxane was put into a two neck round bottom flask (two neck r.b.f) and refluxed and stirred therein under nitrogen for 3 hours. A reaction mixture cooled to room temperature was extracted with MC (methylene chloride)/H₂O and dried using MgSO₄ and then filtered. After being concentrated, a solid is produced with hexane and filtered. As a result, an ivory solid compound 37-1 (11.2 g, 73%) was obtained.

Preparation of Compound 37

A mixed solution of Compound 37-1 (10.0 g, 0.021 mol), 7.11 g (0.023 mol) of 2-bromo-4,6-diphenylpyrimidine, K₂CO₃ (8.69 g, 0.063 mmol), Pd(PPh₃)₄ (1.21 g, 1.05 mmol), and toluene, EtOH, H₂O (100 ml/20 ml/20 ml) was put into a two neck round bottom flask (two neck r.b.f) and refluxed and stirred therein under nitrogen for 12 hours. The solvent of the reaction mixture was filtered and the resultant solid was washed with 200 ml of toluene, 300 ml of hexane, and 300 ml of methanol in sequence. As a result, a white solid compound 37 (9.1 g, 74%) was obtained.

### Preparation Example 5

### Preparation of Compound 63

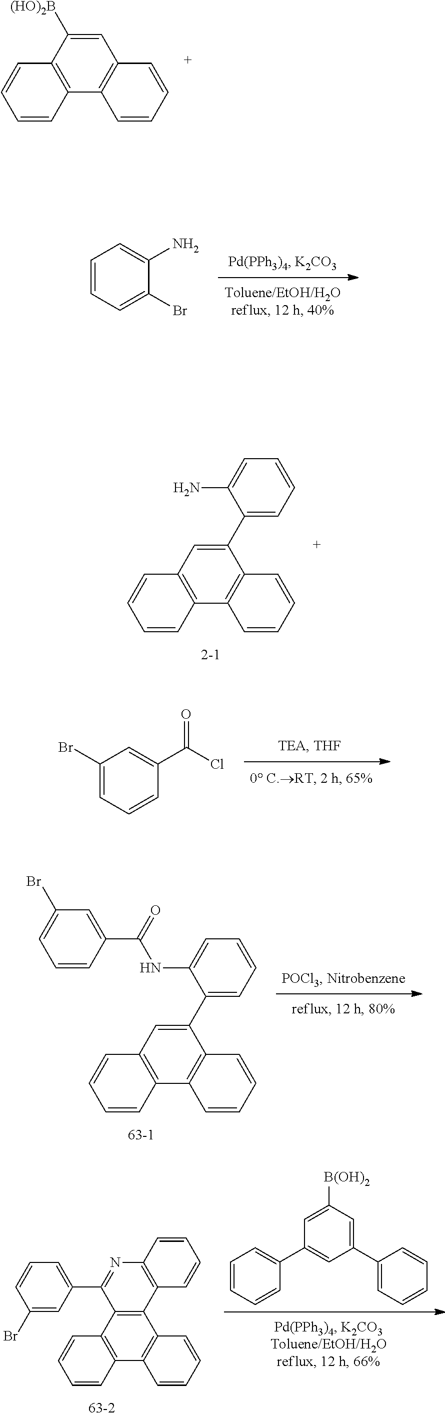

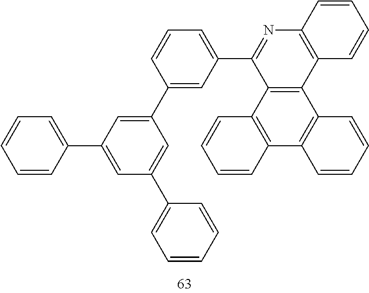 [SMILES: c1ccc(-c2cc(-c3ccccc3)cc(-c3cccc(-c4nc5ccccc5c5c6ccccc6c6ccccc6c45)c3)c2)cc1]

Preparation of Compound 63-1

Compound 2-1 (32 g, 0.119 mol) was dissolved in 200 ml of THF within a one neck round bottom flask (one neck r.b.f) and then triethylamine (49.4 ml, 0.356 mol) was added dropwise thereto at room temperature. The reaction mixture was cooled to 0° C., and 3-bromobenzoyl chloride dissolved in 50 ml of THF was slowly added dropwise thereto. Then, after stirring at room temperature for 2 hours, H₂O was added to terminate the reaction. The solvent was distilled under reduced pressure, and MC (methylene chloride)/H₂O was extracted and dried using MgSO₄ and then filtered. After distillation under reduced pressure and concentration, the resultant reaction product was dissolved in a small amount of MC (methylene chloride) and added with methanol to precipitate a solid. As a result, an ivory solid compound 18-1 (35.0 g, 65%) was obtained.

Preparation of Compound 63-2

Compound 63-1 (35.0 g, 0.077 mol) was dissolved in 200 ml of nitrobenzene within a two neck round bottom flask (two neck r.b.f) and then 1.4 ml (0.015 mol) of POCl₃ (phosphorous oxychloride) was added dropwise thereto at room temperature and refluxed and stirred therein for 12 hours. After the reaction was terminated, the mixed solution was cooled to room temperature and neutralized with NaHCO₃ aqueous solution. Then, MC (methylene chloride)/H₂O was extracted. The resultant reaction product was dried using MgSO₄ and then filtered. After the solvent was distilled under reduced pressure and concentrated, a small amount of MC (methylene chloride) and an excessive amount of hexane were added to precipitate a solid. As a result, a pale yellow solid compound 63-2 (26.7 g, 80%) was obtained.

Preparation of Compound 63

A mixed solution of Compound 63-2 (10.0 g, 0.023 mol), 6.94 g (0.0253 mol) of (3, 5-diphenylphenyl)boronic acid, K₂CO₃ (9.52 g, 0.069 mol), Pd(PPh₃)₄ (1.32 g, 1.15 mol), and toluene, ethanol, H₂O (200 ml/30 ml/30 ml) was put into a two neck round bottom flask (two neck r.b.f) and refluxed and stirred therein under nitrogen for 12 hours. A reaction mixture cooled to room temperature was extracted with MC (methylene chloride)/H₂O and dried using MgSO₄ and then filtered. After being concentrated, the resultant reaction product was separated by column chromatography (SiO₂, hexane:MC (methylene chloride)=2:1). As a result, a white solid compound 63 (8.86 g, 66%) was obtained.

### Preparation Example 6

### Preparation of Compound 73

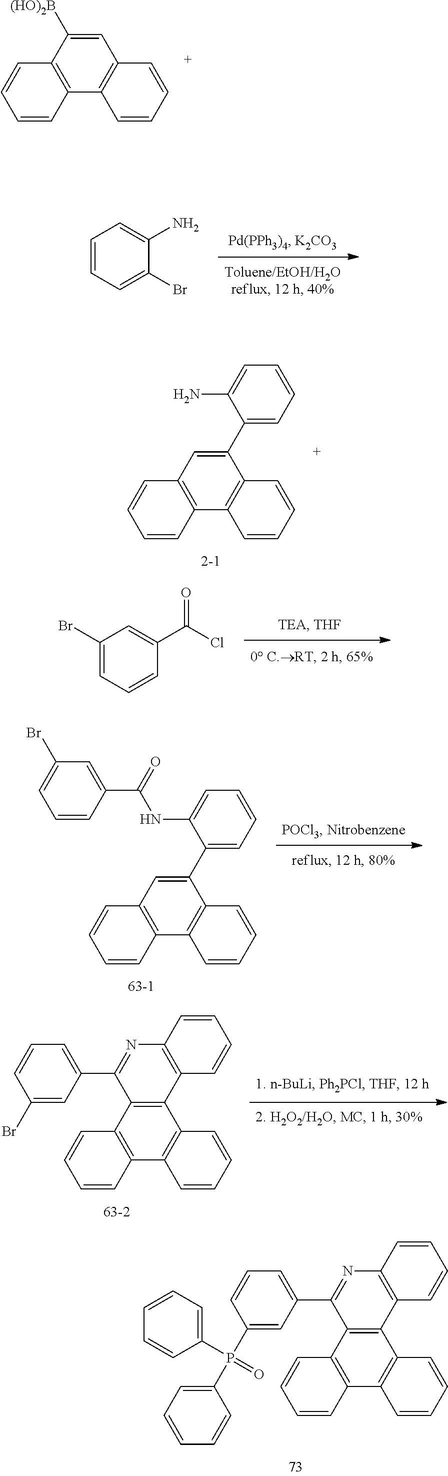

Preparation of Compound 73

Compound 63-2 (10.0 g, 0.023 mol) was dissolved in anhydrous THF (50 ml) within a one neck round bottom flask (one neck r.b.f) under nitrogen and then cooled to −78° C. Then, 12.0 ml (0.0299 mol) of n-butyllithium (2.5 M in hexane) was slowly added dropwise thereto and stirred for 1 hour. 5.37 ml (0.0299 mol) of chlorodiphenylphosphine was added dropwise to the above solution and stirred at room temperature for 12 hours. The reaction mixture was extracted with MC (methylene chloride)/H₂O and distilled under reduced pressure. The reaction mixture was dissolved in 50 ml of MC (methylene chloride) and then stirred with 20 ml of 30% H₂O₂ aqueous solution at room temperature for 1 hour. The reaction mixture was extracted with MC (methylene chloride)/H₂O, and then the concentrated mixture was separated by column chromatography (SiO₂, MC (methylene chloride):methanol=25:1). As a result, a yellow solid compound 73 (3.83 g, 30%) was obtained.

### Preparation Example 7

### Preparation of Compound 92

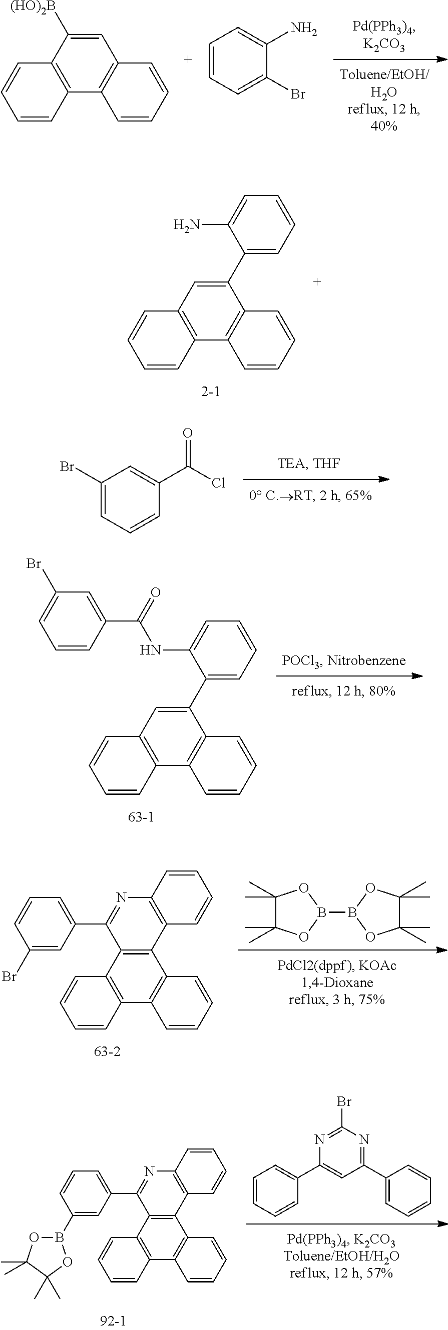

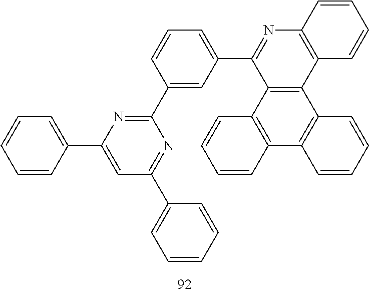 [SMILES: c1ccc(-c2cc(-c3ccccc3)nc(-c3cccc(-c4nc5ccccc5c5c6ccccc6c6ccccc6c45)c3)n2)cc1]

Preparation of Compound 92-1

A mixed solution of Compound 63-2 (14 g, 0.0322 mol), bispinacolate diboron) (9.82 g, 0.0386 mol), 9.48 g (0.0966 mol) of KOAc (potassium acetate), 1.18 g (1.61 mmol) of PdCl₂(dppf), and 1,4-dioxane (250 ml) was put into a two neck round bottom flask (two neck r.b.f) and refluxed and stirred therein under nitrogen for 3 hours. A reaction mixture cooled to room temperature was extracted with MC (methylene chloride)/H₂O and dried using MgSO₄ and then filtered. After being concentrated, a solid is produced with hexane and filtered. As a result, an ivory solid compound 92-1 (11.6 g, 75%) was obtained.

Preparation of Compound 92

A mixed solution of Compound 92-1 (11.6 g, 0.024 mol), 8.25 g (0.0265 mol) of 2-bromo-4,6-diphenylpyrimidine, K₂CO₃ (9.94 g, 0.072 mmol), Pd(PPh₃)₄ (1.39 g, 1.2 mmol), and toluene, ethanol, H₂O (200 ml/30 ml/30 ml) was put into a two neck round bottom flask (two neck r.b.f) and refluxed and stirred therein under nitrogen for 12 hours. The solvent of the reaction mixture was filtered and the resultant solid was washed with 200 ml of toluene, 300 ml of hexane, and 300 ml of methanol in sequence. As a result, a white solid compound 92 (8.04 g, 57%) was obtained.

### Preparation Example 8

### Preparation of Compound 111

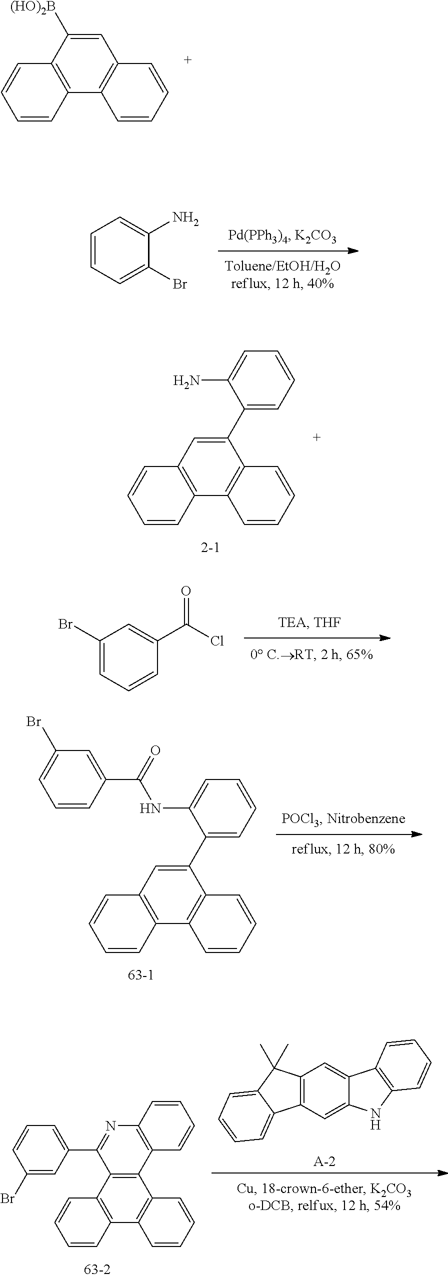

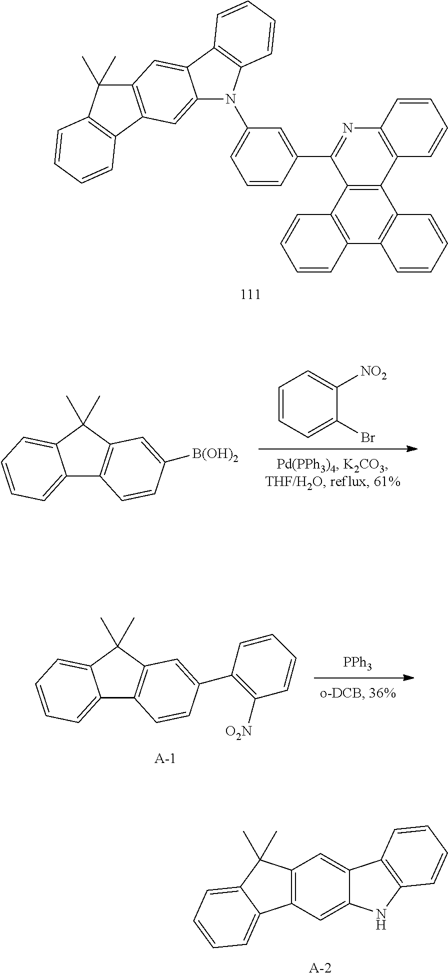

Preparation of Compound A-1

A mixture of 25.9 g (0.108 mol) of (9,9-dimethyl-9H-fluoren-2-yl)boronic acid, 20 g (0.099 mol) of 1-bromo-2-nitrobenzene, Pd(PPh₃)₄ (5.7 g, 4.95 mmol), K₂CO₃ (27.3 g, 0.198 mol), and THF (250 ml)/H₂O (50 ml) was put into a one neck round bottom flask (one neck r.b.f) and refluxed and stirred for 24 hours. After a water layer was removed, an organic layer was dried using MgSO₄. After being concentrated, the resultant reaction product was separated by column chromatography (SiO₂, hexane:MC (methylene chloride)=2:1). As a result, a yellow solid compound A-1 (21 g, 61%) was obtained.

Preparation of Compound A-2

A mixture of Compound A-1 (20 g, 0.0634 mmol), PPh₃ (49.8 g, 0.190 mol), and o-dichlorobenzene (o-DCB) (300 ml) was put into a one neck round bottom flask (one neck r.b.f) and refluxed and stirred under nitrogen for 18 hours. After o-dichlorobenzene (o-DCB) was distilled under reduced pressure and removed, the resultant reaction product was separated by column chromatography (SiO₂, hexane:MC (methylene chloride)=3:1). As a result, a white solid compound A-2 (6.6 g, 36%) was obtained.

Preparation of Compound 111

A mixture of Compound 63-2 (8.0 g, 0.0184 mol), Compound A-2 (4.74 g, 0.0167 mol), Cu (0.58 g, 9.2 mol), 0.97 g (3.68 mmol) of 18-crown-6-ether, K₂CO₃ (12.7 g, 0.092 mol), and o-dichlorobenzene (o-DCB) (80 ml) was put into a one neck round bottom flask (one neck r.b.f) and refluxed and stirred under nitrogen for 12 hours. After o-dichlorobenzene (o-DCB) was distilled under reduced pressure and removed, the resultant reaction product was separated by column chromatography (SiO₂, hexane:MC (methylene chloride)=4:1). As a result, a white solid compound 111 (6.3 g, 54%) was obtained.

### Preparation Example 9

### Preparation of Compound 130

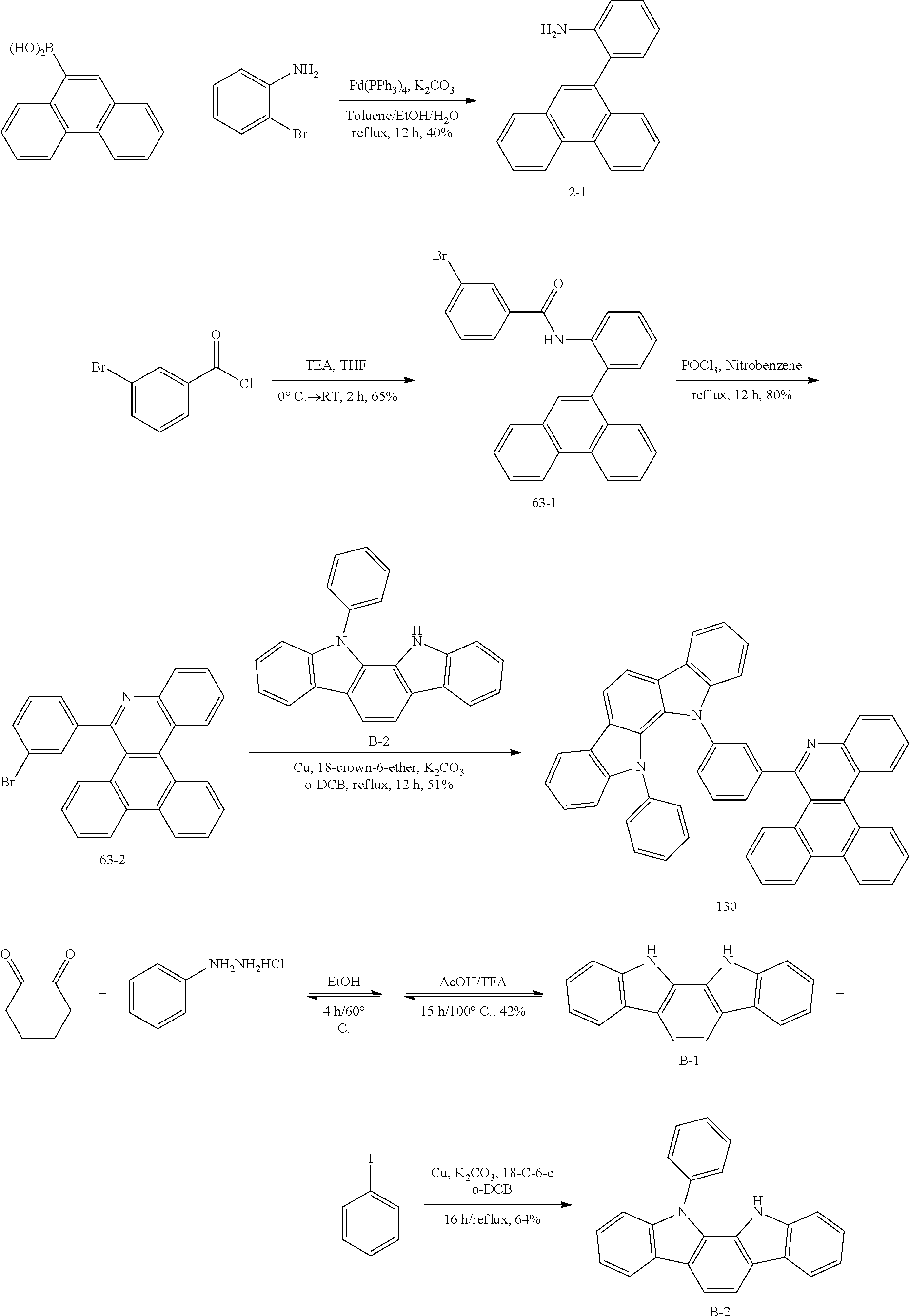

Preparation of Compound B-1

Sulfuric acid (1.4 mL, 0.0374 mol) was slowly added dropwise to a mixture of 30.0 g (0.374 mol) of 1,2-dicyclohexanone, 77.37 g (0.749 mol) of phenylhydrazine hydrochloride, and ethanol (1000 ml) in a one neck round bottom flask (one neck r.b.f) under nitrogen and stirred at 60° C. for 4 hours. The mixed solution cooled to room temperature was filtered, and a light brown solid (69 g, 93%) was obtained. Trifluoroacetic acid (46.5 mL, 0.6 mol) was added to a mixture of the above solid (68.9 g, 0.25 mol) and acetic acid (700 ml) within a one neck round bottom flask (one neck r.b.f) and stirred at 100° C. for 15 hours. The mixed solution cooled to room temperature was washed with acetic acid and hexane and filtered. As a result, an ivory solid B-1 (27.3 g, 42%) was obtained.

Preparation of Compound B-2

A mixture of Compound B-1 (2.1 g, 0.0082 mol), iodobenzene (2.5 g, 0.013 mol), Cu (0.312 g, 0.0049), 18-crown-6-ether (0.433 g, 0.0016 mol), K₂CO₃ (3.397 g, 0.0246 mol), and o-dichlorobenzene (o-DCB) (20 ml) was refluxed and stirred in a two neck round bottom flask (two neck r.b.f) under nitrogen for 16 hours. The mixed solution cooled to room temperature was extracted with MC (methylene chloride)/H₂O and concentrated and separated by column chromatography (SiO₂, hexane:ethyl acetate=10:1). As a result, a white solid compound B-2 was obtained (1.76 g, 64%).

Preparation of Compound 130

A mixture of Compound 63-2 (5.0 g, 0.0115 mmol), Compound B-2 (3.48 g, 0.01 mmol), Cu (0.32 g, 5.0 mmol), 18-crown-6-ether (0.53 g, 2.0 mmol), K₂CO₃ (6.9 g, 0.05 mol), and o-dichlorobenzene (o-DCB) (30 ml) was refluxed and stirred in a two neck round bottom flask (two neck r.b.f) under nitrogen for 12 hours. After o-dichlorobenzene (o-DCB) was distilled under reduced pressure and removed, the resultant reaction product was separated by column chromatography (SiO₂, hexane:MC (methylene chloride)=3:1). As a result, a solid compound 130 (3.5 g, 51%) was obtained.

### Preparation Example 10

### Preparation of Compound 181

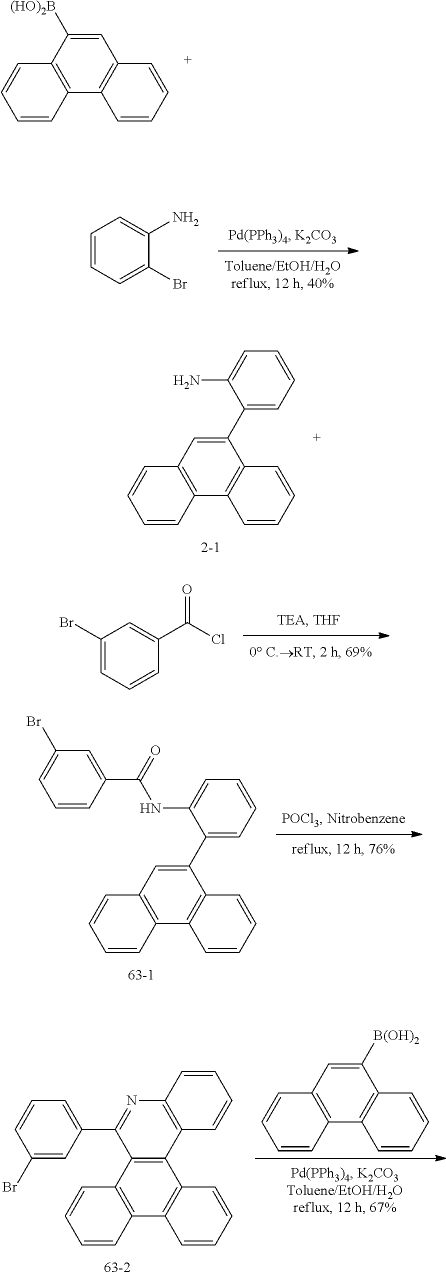

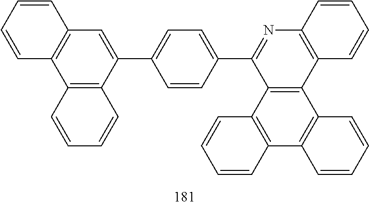 [SMILES: c1ccc2c(c1)cc(-c1ccc(-c3nc4ccccc4c4c5ccccc5c5ccccc5c34)cc1)c1ccccc12]

Preparation of Compound 181

A mixed solution of Compound 63-2 (10.0 g, 0.023 mol), phenanthrene-9-yl boronic acid (5.6 g, 0.0253 mol), K₂CO₃ (9.5 g, 0.069 mol), Pd(PPh₃)₄ (1.33 g, 1.15 mmol), and toluene, ethanol, H₂O (200 ml/30 ml/30 ml) was put into a two neck round bottom flask (two neck r.b.f) and refluxed and stirred therein for 12 hours. A reaction mixture cooled to room temperature was extracted with MC (methylene chloride)/H₂O and dried using MgSO₄ and then filtered. After being concentrated, the resultant reaction product was separated by column chromatography (SiO₂, hexane:MC (methylene chloride)=1:1). As a result, a white solid compound 181 (8.19 g, 67%) was obtained.

### Preparation Example 11

### Preparation of Compound 209

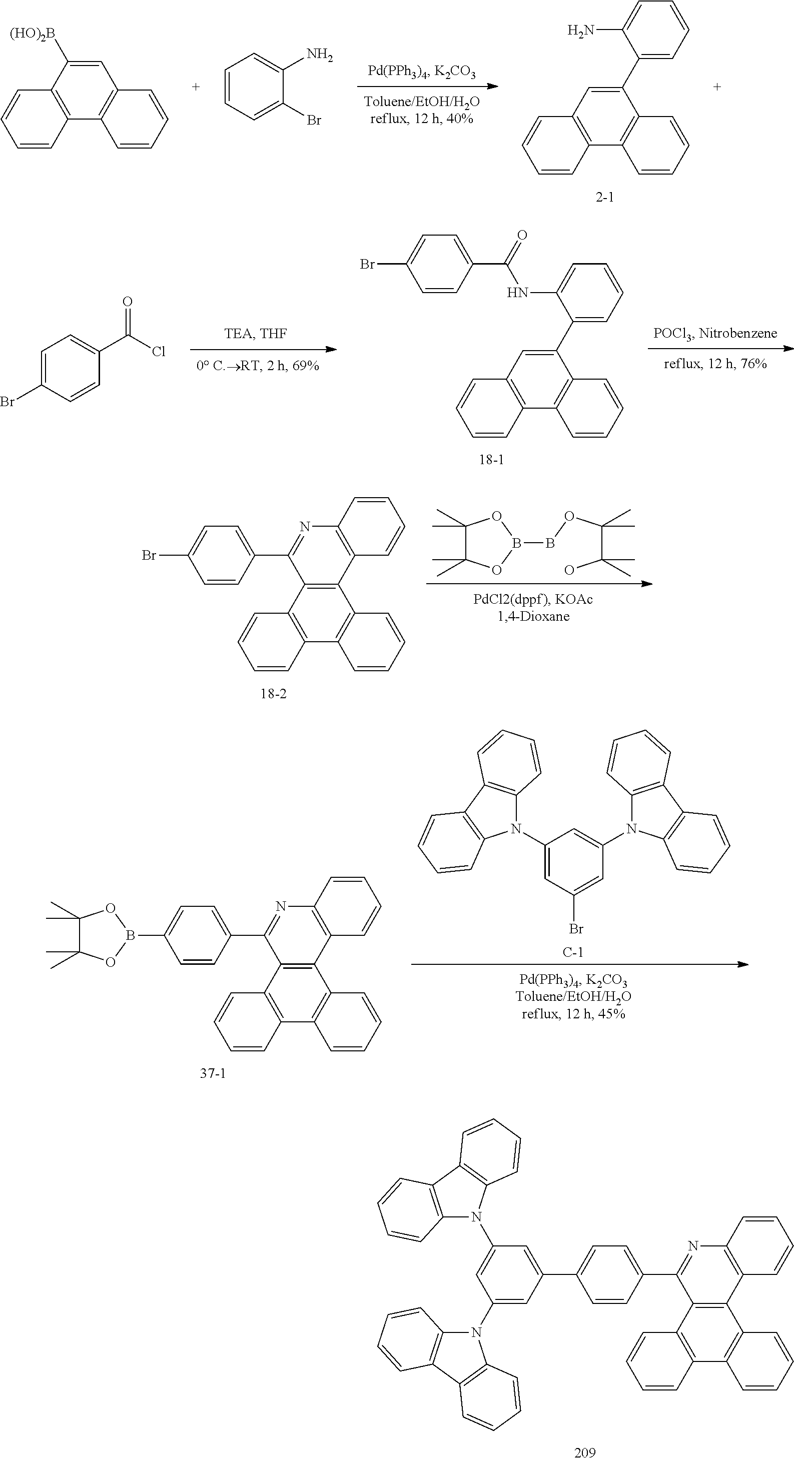

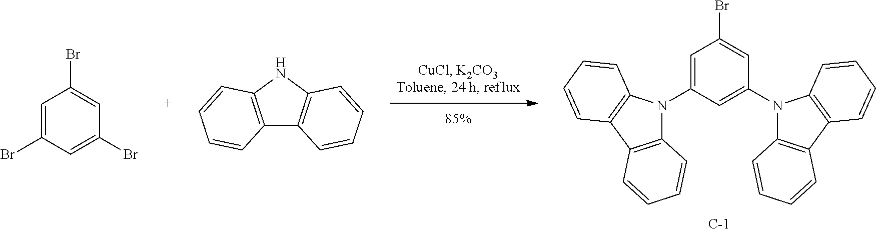

Preparation of Compound C-1

A mixture of 1,3,5-tribromobenzene (10.0 g, 0.031 mol), 9H-carbazole (10.37 g, 0.062 mol), CuCl (0.3 g, 3.1 mmol), K₂CO₃ (8.56 g, 0.062 mol), and toluene (400 ml) was refluxed and stirred in a two neck round bottom flask (two neck r.b.f) under nitrogen for 24 hours. The mixed solution cooled to room temperature was extracted with MC (methylene chloride)/H₂O and dried using MgSO₄ and then filtered. After being concentrated, the resultant reaction product was separated by column chromatography (SiO₂, hexane:MC (methylene chloride)=1:1). As a result, a white solid compound C-1 was obtained (9.75 g, 65%).

Preparation of Compound 209

A mixed solution of Compound 37-1 (10.0 g, 0.021 mol), Compound C-1 (9.2 g, 0.019 mol), K₂CO₃ (8.7 g, 0.063 mol), Pd(PPh₃)₄ (1.21 g, 1.05 mmol), and toluene, ethanol, H₂O (200 ml/30 ml/30 ml) was refluxed and stirred in a two neck round bottom flask (two neck r.b.f) under nitrogen for 12 hours. The mixed solution cooled to room temperature was extracted with MC (methylene chloride)/H₂O and dried using MgSO₄ and then filtered. After being concentrated, the resultant reaction product was separated by column chromatography (SiO₂, hexane:MC (methylene chloride)=1:1). As a result, a white solid compound 209 (7.2 g, 45%) was obtained.

### Preparation Example 12

### Preparation of Compounds 220, 224, 228, 232, 236, 240, and 244

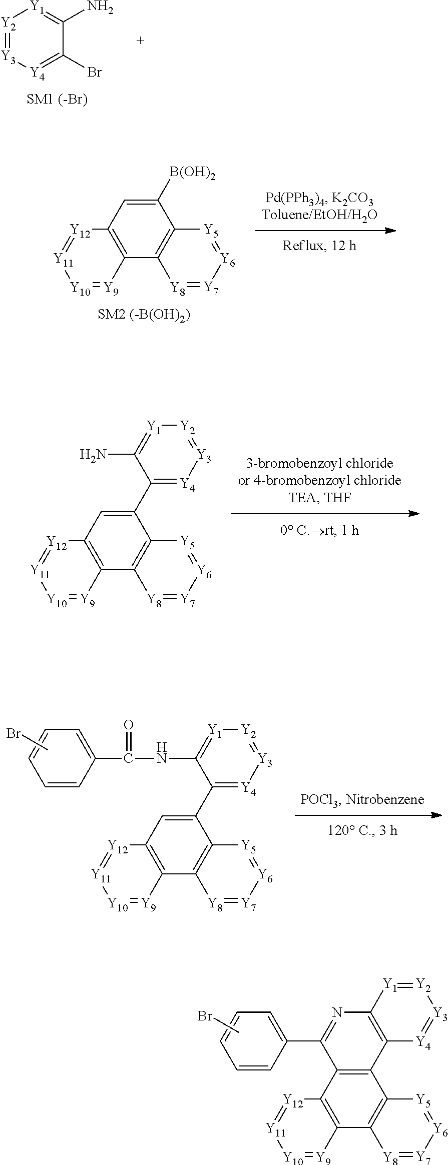

### Preparation Example 13

### Preparation of Compounds 248, 252, 256, 260, 264, 268, and 272

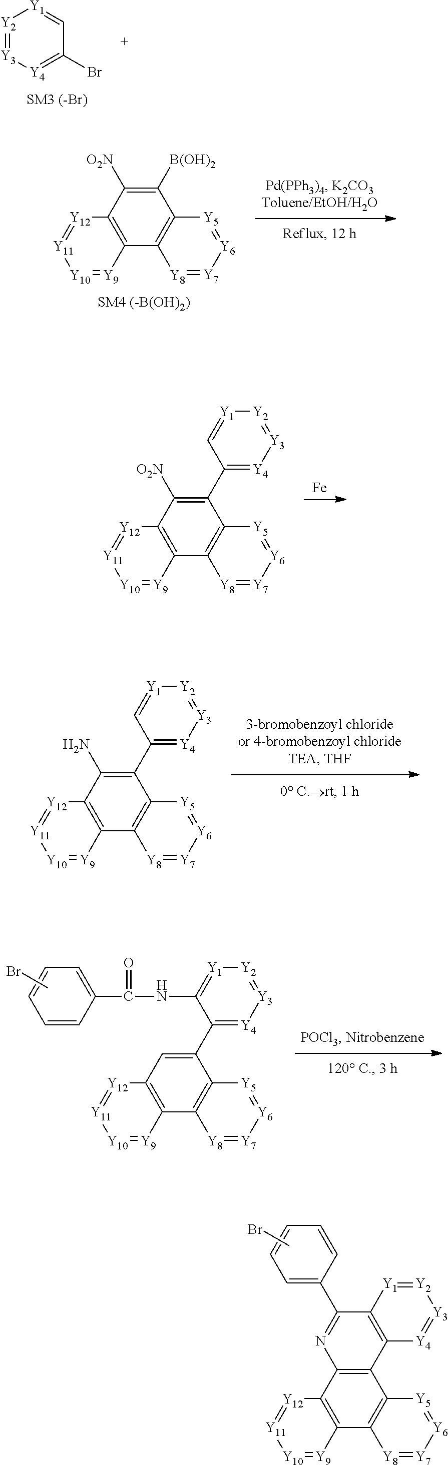

In the preparation examples 12 and 13, Compounds 220 to 275 of the present invention can be prepared by modifying a starting material 1 (SM1 (—Br)), a starting material 2 (SM2 (—B(OH)₂), a starting material 3 (SM3 (—Br)), and a starting material 4 (SM4 (—B(OH)₂). As the starting materials 1 to 4, compounds respectively having the structures as listed in the following Table 1 were used to prepare Compounds 220, 224, 228, 232, 236, 240, 244, 248, 252, 256, 260, 264, 268, and 272. Herein, Y₁ to Y₁₂ are —CH, but Y₁ to Y₁₂ in the starting materials 1 to 4 can be modified.

TABLE 1

| Com- | | |
|---|---|---|
| pound | SM1 (—Br) | SM2 (—B(OH)₂) |
| 220 | 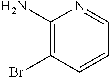 | 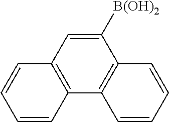 [SMILES: OB(O)c1cc2ccccc2c2ccccc12] |
| 224 | 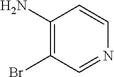 | 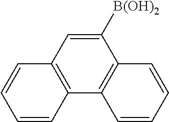 [SMILES: OB(O)c1cc2ccccc2c2ccccc12] |
| 228 | 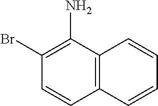 [SMILES: Nc1c(Br)ccc2ccccc12] | 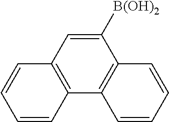 [SMILES: OB(O)c1cc2ccccc2c2ccccc12] |
| 232 | 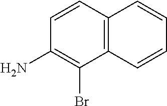 [SMILES: Nc1ccc2ccccc2c1Br] | 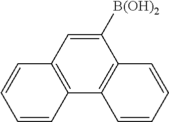 [SMILES: OB(O)c1cc2ccccc2c2ccccc12] |
| 236 | 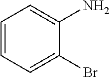 | 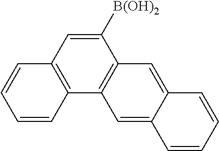 [SMILES: OB(O)c1cc2ccccc2c2cc3ccccc3cc12] |
| 240 | 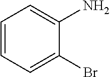 | 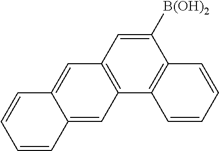 [SMILES: OB(O)c1cc2cc3ccccc3cc2c2ccccc12] |
| 244 | 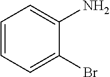 | 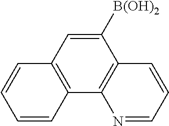 [SMILES: OB(O)c1cc2ccccc2c2ncccc12] |
| 248 | 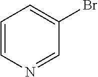 | 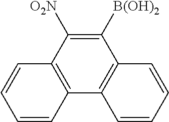 [SMILES: O=[N+]([O-])c1c(B(O)O)c2ccccc2c2ccccc12] |
| 252 | 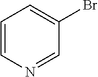 | 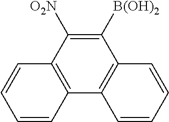 [SMILES: O=[N+]([O-])c1c(B(O)O)c2ccccc2c2ccccc12] |
| 256 | 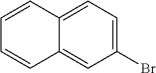 [SMILES: Brc1ccc2ccccc2c1] | 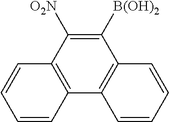 [SMILES: O=[N+]([O-])c1c(B(O)O)c2ccccc2c2ccccc12] |
| 260 | 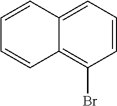 [SMILES: Brc1cccc2ccccc12] | 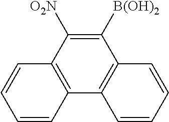 [SMILES: O=[N+]([O-])c1c(B(O)O)c2ccccc2c2ccccc12] |
| 264 | 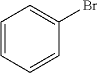 | 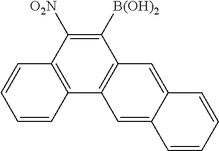 [SMILES: O=[N+]([O-])c1c(B(O)O)c2cc3ccccc3cc2c2ccccc12] |
| 268 | 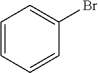 | 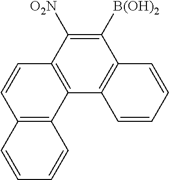 [SMILES: O=[N+]([O-])c1c(B(O)O)c2ccccc2c2c1ccc1ccccc12] |
| 272 | 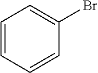 | 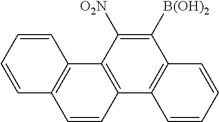 [SMILES: O=[N+]([O-])c1c(B(O)O)c2ccccc2c2ccc3ccccc3c12] |

### Preparation Example 14

### Preparation of Compound 324

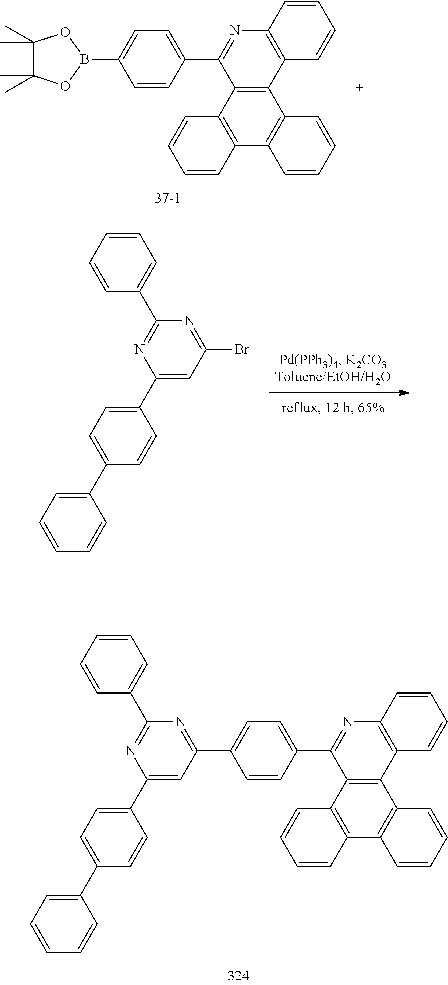 [SMILES: CC1(C)OB(c2ccc(-c3nc4ccccc4c4c5ccccc5c5ccccc5c34)cc2)OC1(C)C ; Brc1cc(-c2ccc(-c3ccccc3)cc2)nc(-c2ccccc2)n1 ; c1ccc(-c2ccc(-c3cc(-c4ccc(-c5nc6ccccc6c6c7ccccc7c7ccccc7c56)cc4)nc(-c4ccccc4)n3)cc2)cc1]

Preparation of Compound 324

A mixed solution of Compound 37-1 (10.0 g, 0.0208 mol), 4-([1,1′-biphenyl]-4-yl)-6-bromo-2-phenylpyrimidine (9.65 g, 0.249 mol), K₂CO₃ (8.61 g, 0.062 mol), Pd(PPh₃)₄ (1.20 g, 0.00104 mol), and toluene, ethanol, H₂O (200 ml/30 ml/30 ml) was refluxed and stirred in a two neck round bottom flask (two neck r.b.f) under nitrogen for 12 hours. A precipitated solid was filtered with the reaction mixture cooled to room temperature and washed with toluene and methanol. The filtered solid was dissolved in an excessive amount of hot toluene and allowed to pass through silica gel and then concentrated. The resultant reaction product was dissolved in a small amount of EA and in a small amount of 1,2-dichloroethane and then filtered. As a result, a white solid compound 324 (8.95 g, 65%) was obtained.

### Preparation Example 15

### Preparation of Compound 337

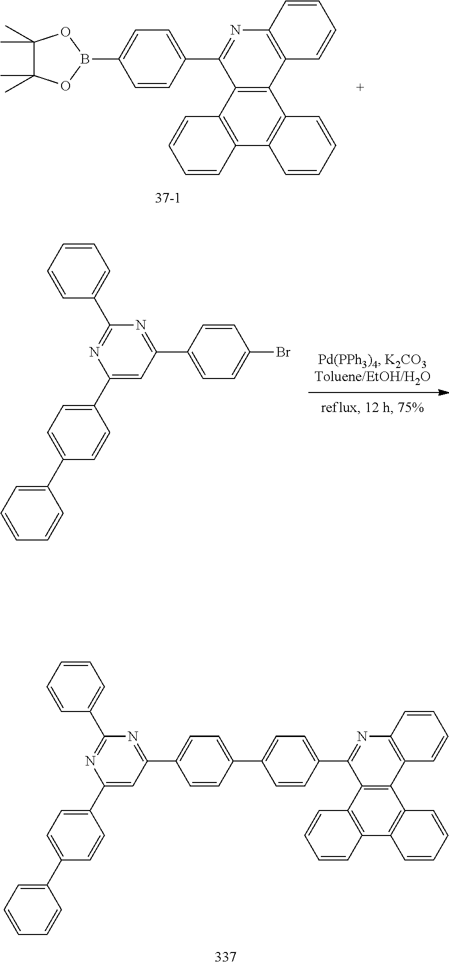 [SMILES: CC1(C)OB(c2ccc(-c3nc4ccccc4c4c5ccccc5c5ccccc5c34)cc2)OC1(C)C ; Brc1ccc(-c2cc(-c3ccc(-c4ccccc4)cc3)nc(-c3ccccc3)n2)cc1 ; c1ccc(-c2ccc(-c3cc(-c4ccc(-c5ccc(-c6nc7ccccc7c7c8ccccc8c8ccccc8c67)cc5)cc4)nc(-c4ccccc4)n3)cc2)cc1]

Preparation of Compound 337

A mixed solution of Compound 37-1 (10.0 g, 0.0208 mol), 4-([1,1′-biphenyl]-4-yl)-6-(4-bromophenyl)-2-phenylpyrimidine (11.54 g, 0.249 mol), K₂CO₃ (8.61 g, 0.062 mol), Pd(PPh₃)₄ (1.20 g, 0.00104 mol), and toluene, ethanol, H₂O (200 ml/30 ml/30 ml) was refluxed and stirred in a two neck round bottom flask (two neck r.b.f) under nitrogen for 12 hours. A precipitated solid was filtered with the reaction mixture cooled to room temperature and washed with toluene and methanol. The filtered solid was dissolved in an excessive amount of hot toluene and allowed to pass through silica gel and then concentrated. The resultant reaction product was dissolved in a small amount of EA and in a small amount of 1,2-dichloroethane and then filtered. As a result, a white solid compound 337 (11.5 g, 75%) was obtained.

### Preparation Example 16

### Preparation of Compound 376

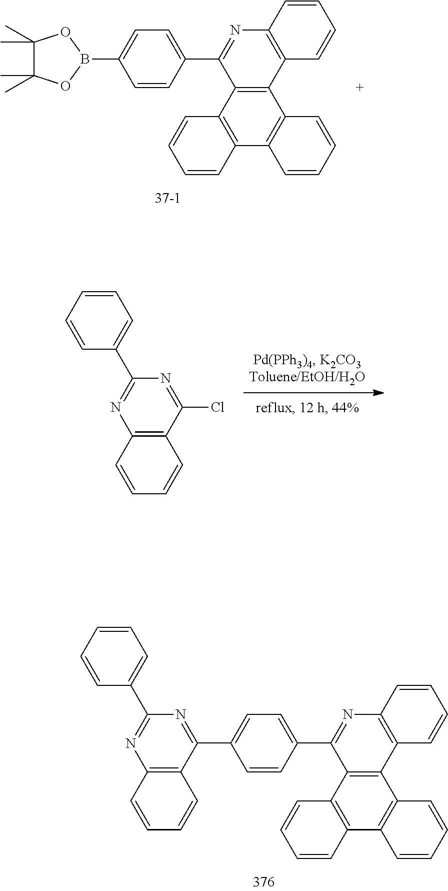 [SMILES: CC1(C)OB(c2ccc(-c3nc4ccccc4c4c5ccccc5c5ccccc5c34)cc2)OC1(C)C ; Clc1nc(-c2ccccc2)nc2ccccc12 ; c1ccc(-c2nc(-c3ccc(-c4nc5ccccc5c5c6ccccc6c6ccccc6c45)cc3)c3ccccc3n2)cc1]

Preparation of Compound 376

A mixed solution of Compound 37-1 (10.0 g, 0.0208 mol), 4-chloro-2-phenylquinazoline (5.99 g, 0.0249 mol), K₂CO₃ (8.61 g, 0.062 mmol), Pd(PPh₃)₄ (1.20 g, 0.00104 mol), and toluene, ethanol, H₂O (200 ml/30 ml/30 ml) was refluxed and stirred in a two neck round bottom flask (two neck r.b.f) under nitrogen for 12 hours. The reaction mixture cooled to room temperature was extracted with MC (methylene chloride)/H₂O and dried using MgSO₄ and then filtered. After being concentrated, the resultant reaction product was separated by column chromatography (SiO₂, hexane:MC=2:1). As a result, a white solid compound 376 (8.61 g, 44%) was obtained.

### Preparation Example 17

### Preparation of Compound 386

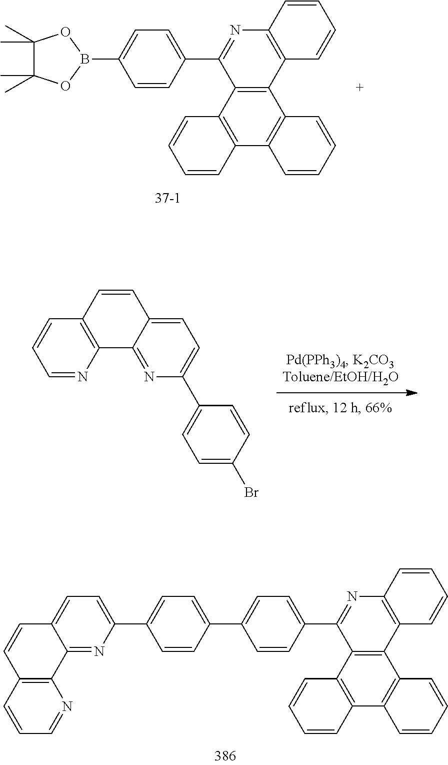 [SMILES: CC1(C)OB(c2ccc(-c3nc4ccccc4c4c5ccccc5c5ccccc5c34)cc2)OC1(C)C ; Brc1ccc(-c2ccc3ccc4cccnc4c3n2)cc1 ; c1cnc2c(c1)ccc1ccc(-c3ccc(-c4ccc(-c5nc6ccccc6c6c7ccccc7c7ccccc7c56)cc4)cc3)nc12]

Preparation of Compound 386

A mixed solution of Compound 37-1 (10.0 g, 0.0208 mol), 2-(4-bromophenyl)-1, 10-phenanthroline (8.35 g, 0.0249 mol), K₂CO₃ (8.61 g, 0.062 mol), Pd(PPh₃)₄ (1.20 g, 0.00104 mol), and toluene, ethanol, H₂O (200 ml/30 ml/30 ml) was refluxed and stirred in a two neck round bottom flask (two neck r.b.f) under nitrogen for 12 hours. A precipitated solid was filtered with the reaction mixture cooled to room temperature and washed with toluene and methanol. The filtered solid was dissolved in an excessive amount of hot toluene and allowed to pass through silica gel and then concentrated. The resultant reaction product as being hot was stirred for 3 or more hours in EA and 1,2-dichloroethane and then filtered. As a result, a white solid compound 386 (8.37 g, 66%) was obtained.

### Preparation Example 18

### Preparation of Compound 388

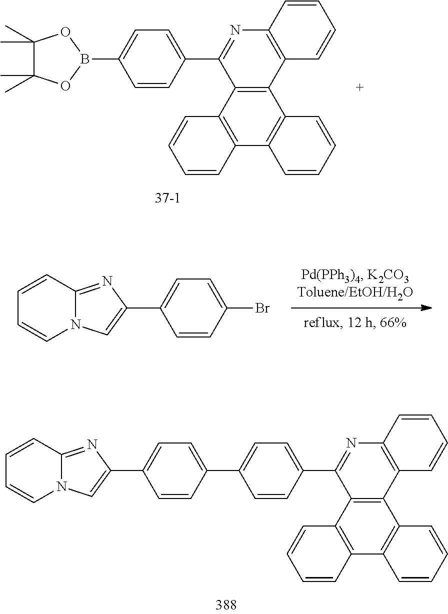 [SMILES: CC1(C)OB(c2ccc(-c3nc4ccccc4c4c5ccccc5c5ccccc5c34)cc2)OC1(C)C ; Brc1ccc(-c2cn3ccccc3n2)cc1 ; c1ccc2c(c1)nc(-c1ccc(-c3ccc(-c4cn5ccccc5n4)cc3)cc1)c1c3ccccc3c3ccccc3c21]

Preparation of Compound 388

A mixed solution of Compound 37-1 (10.0 g, 0.0208 mol), 2-(4-bromophenyl)imidazo[1,2-a]pyridine (6.80 g, 0.0249 mol), K₂CO₃ (8.61 g, 0.062 mmol), Pd(PPh₃)₄ (1.2 g, 0.00104 mol), and toluene, ethanol, H₂O (200 ml/30 ml/30 ml) was refluxed and stirred in a two neck round bottom flask (two neck r.b.f) under nitrogen for 12 hours. The reaction mixture cooled to room temperature was extracted with MC (methylene chloride)/H₂O and dried using MgSO₄ and allowed to pass through silica gel. After being concentrated, the resultant reaction product was dissolved in a small amount of EA, in a small amount of 1,2-dichloroethane, and in a small amount of methanol and stirred for 3 or more hours and then filtered. As a result, a white compound 388 (5.92 g, 66%) was obtained.

### Preparation Example 19

### Preparation of Compound 396

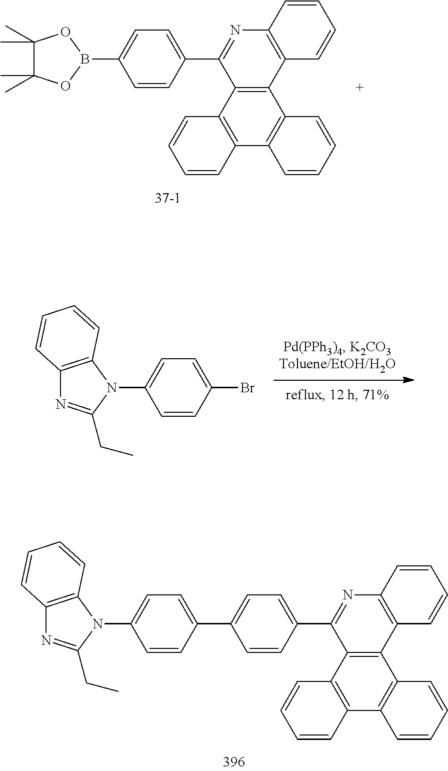 [SMILES: CC1(C)OB(c2ccc(-c3nc4ccccc4c4c5ccccc5c5ccccc5c34)cc2)OC1(C)C ; CCc1nc2ccccc2n1-c1ccc(Br)cc1 ; CCc1nc2ccccc2n1-c1ccc(-c2ccc(-c3nc4ccccc4c4c5ccccc5c5ccccc5c34)cc2)cc1]

Preparation of Compound 396

A mixed solution of Compound 37-1 (10.0 g, 0.0208 mol), 1-(4-bromophenyl)-2-ethyl-1H-benzo[d]imidazole (7.50 g, 0.0249 mol), K₂CO₃ (8.61 g, 0.062 mmol), Pd(PPh₃)₄ (1.20 g, 0.00104 mol), and toluene, ethanol, H₂O (200 ml/30 ml/30 ml) was refluxed and stirred in a two neck round bottom flask (two neck r.b.f) under nitrogen for 12 hours. The reaction mixture cooled to room temperature was extracted with MC (methylene chloride)/H₂O and dried using MgSO₄ and filtered. After being concentrated, the resultant reaction product was separated by column chromatography (SiO₂, hexane:MC=2:1). As a result, a white solid compound 396 (8.50 g, 71%) was obtained.

### Preparation Example 20

### Preparation of Compound 421

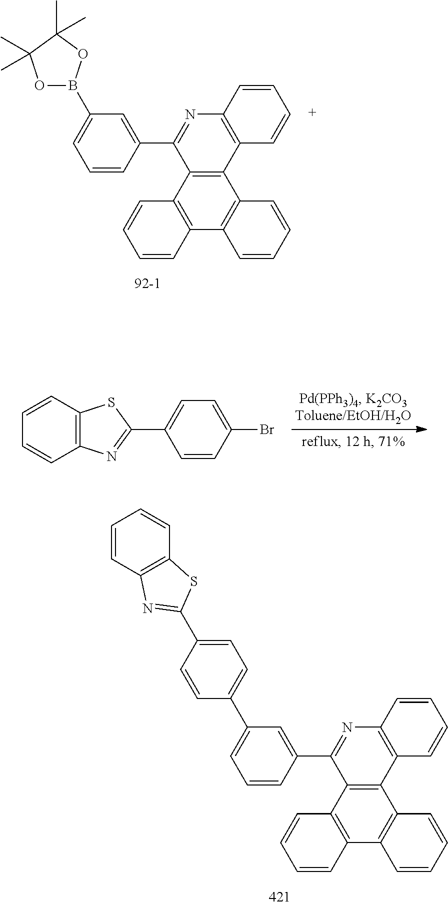

Preparation of Compound 421

A mixed solution of Compound 92-1 (10.0 g, 0.0208 mol), 2-(4-bromophenyl)benzo[d]thiazole) (7.22 g, 0.0249 mol), K₂CO₃ (8.61 g, 0.062 mmol), Pd(PPh₃)₄ (1.20 g, 0.00104 mol), and toluene, ethanol, H₂O (200 ml/30 ml/30 ml) was refluxed and stirred in a two neck round bottom flask (two neck r.b.f) under nitrogen for 12 hours. The reaction mixture cooled to room temperature was extracted with MC (methylene chloride)/H₂O and dried using MgSO₄ and allowed to pass through silica gel. After being concentrated, the resultant reaction product was dissolved in a small amount of EA, in a small amount of 1,2-dichloroethane, and in a small amount of methanol and stirred for 3 or more hours and then filtered. As a result, a white compound 421 (8.22 g, 70%) was obtained.

### Preparation Example 21

### Preparation of Compound 422

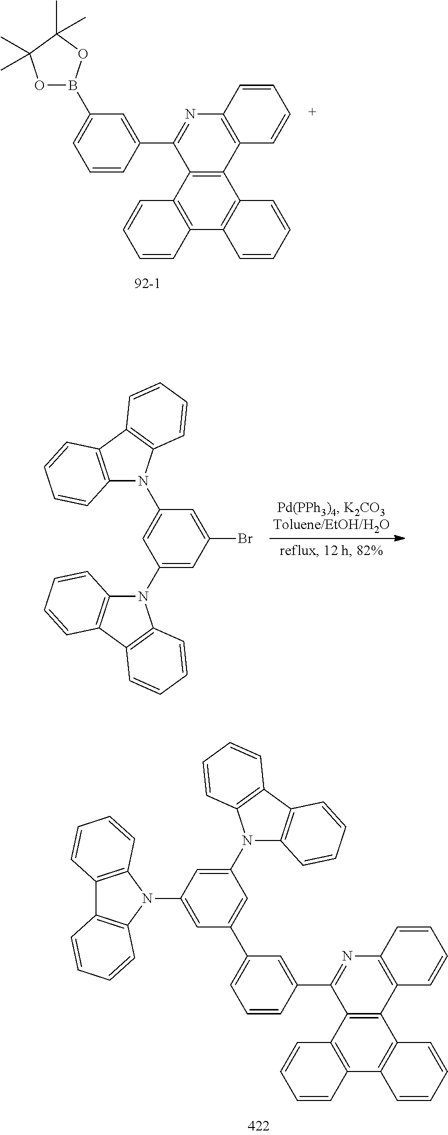

Preparation of Compound 422

A mixed solution of Compound 92-1 (10.0 g, 0.0208 mol), 9,9′-(5-bromo-1,3-phenylene)bis(9H-carbazole) (12.1 g, 0.0249 mol), K₂CO₃ (8.61 g, 0.062 mol), Pd(PPh₃)₄ (1.20 g, 0.00104 mol), and toluene, ethanol, H₂O (200 ml/30 ml/30 ml) was refluxed and stirred in a two neck round bottom flask (two neck r.b.f) under nitrogen for 12 hours. A precipitated solid was filtered with the reaction mixture cooled to room temperature and washed with toluene and methanol. The filtered solid was dissolved in an excessive amount of hot toluene and allowed to pass through silica gel and then concentrated. The resultant reaction product was dissolved in a small amount of EA and in a small amount of 1,2-dichloroethane and then filtered. As a result, a white solid compound 422 (12.98 g, 82%) was obtained.

### Preparation Example 22

### Preparation of Compound 465

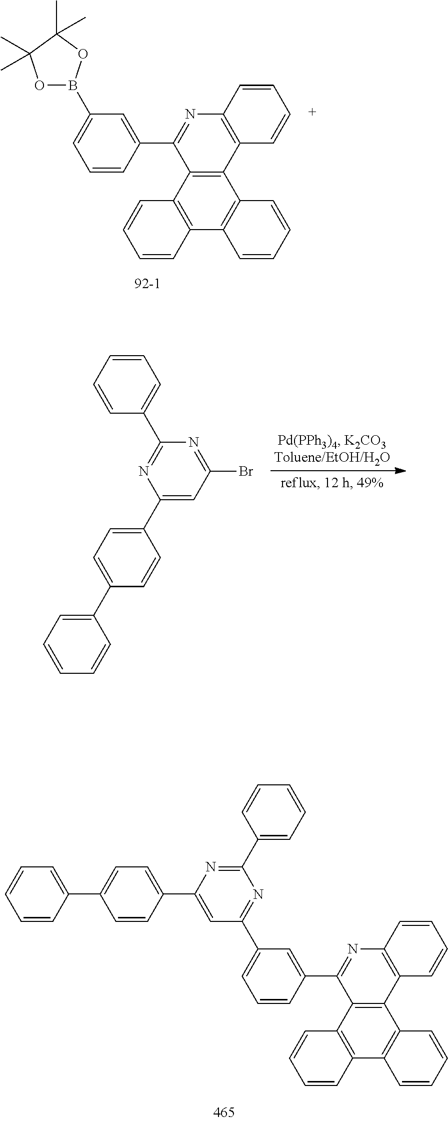

Preparation of Compound 465

A mixed solution of Compound 92-1 (10.0 g, 0.0208 mol), 4-([1,1′-biphenyl]-4-yl)-6-bromo-2-phenylpyrimidine (9.64 g, 0.0249 mol), K₂CO₃ (8.61 g, 0.062 mmol), Pd(PPh₃)₄ (1.20 g, 0.00104 mol), and toluene, ethanol, H₂O (200 ml/30 ml/30 ml) was refluxed and stirred in a two neck round bottom flask (two neck r.b.f) under nitrogen for 12 hours. The reaction mixture cooled to room temperature was extracted with MC (methylene chloride)/H₂O and dried using MgSO₄ and then filtered. After being concentrated, the resultant reaction product was separated by column chromatography (SiO₂, hexane:MC=2:1). As a result, a white solid compound 465 (6.75 g, 49%) was obtained.

### Preparation Example 23

### Preparation of Compound 477

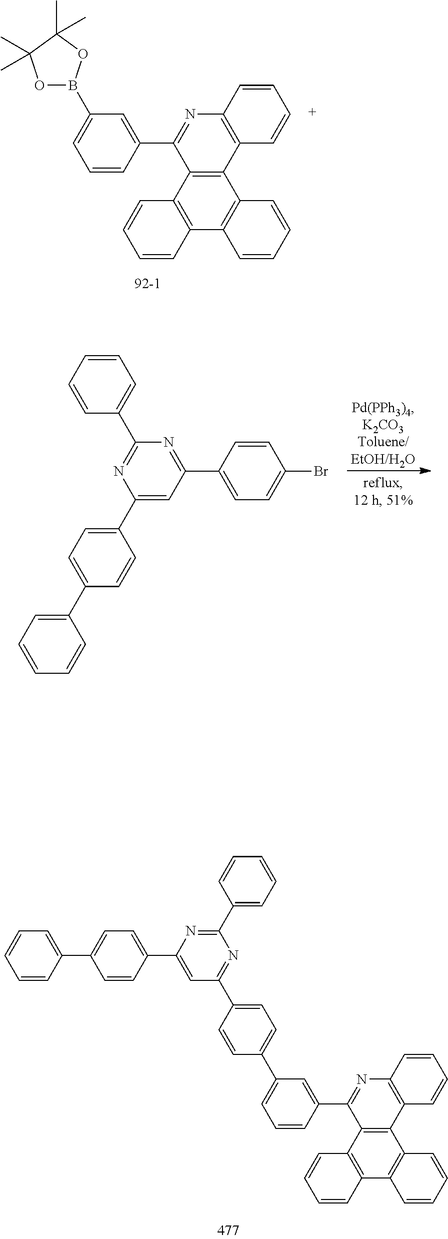

Preparation of Compound 477

A mixed solution of Compound 92-1 (10.0 g, 0.0208 mol), 4-([1,1′-biphenyl]-4-yl)-6-(4-bromophenyl)-2-phenylpyrimidine (11.5 g, 0.0249 mol), K₂CO₃ (8.61 g, 0.062 mmol), Pd(PPh₃)₄ (1.20 g, 0.00104 mol), and toluene, ethanol, H₂O (200 ml/30 ml/30 ml) was refluxed and stirred in a two neck round bottom flask (two neck r.b.f) under nitrogen for 12 hours. The reaction mixture cooled to room temperature was extracted with MC (methylene chloride)/H₂O and dried using MgSO₄ and allowed to pass through silica gel. After being concentrated, the resultant reaction product was dissolved in a small amount of EA, in a small amount of 1,2-dichloroethane, and in a small amount of methanol and stirred for 3 or more hours and then filtered. As a result, a white compound 477 (7.83 g, 51%) was obtained.

### Preparation Example 24

### Preparation of Compound 518

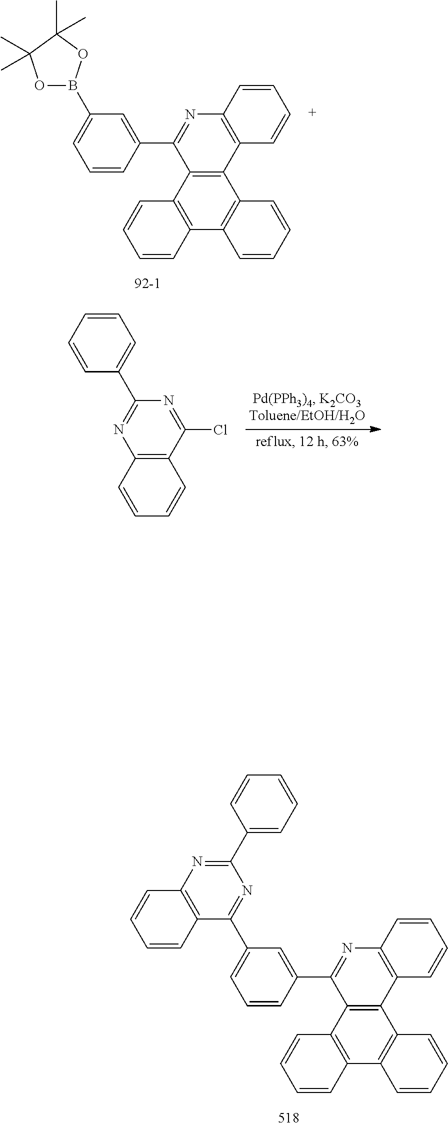

Preparation of Compound 518

A mixed solution of Compound 92-1 (10.0 g, 0.0208 mol), 4-chloro-2-phenylquinazoline (5.99 g, 0.0249 mol), K₂CO₃ (8.61 g, 0.062 mol), Pd(PPh₃)₄ (1.20 g, 0.00104 mol), and toluene, ethanol, H₂O (200 ml/30 ml/30 ml) was refluxed and stirred in a two neck round bottom flask (two neck r.b.f) under nitrogen for 12 hours. The reaction mixture cooled to room temperature was extracted with MC (methylene chloride)/H₂O and dried using MgSO₄ and then filtered. After being concentrated, the resultant reaction product was separated by column chromatography (SiO₂, hexane:MC=2:1). As a result, a white solid compound 518 (7.33 g, 63%) was obtained.

### Preparation Example 25

### Preparation of Compound 528

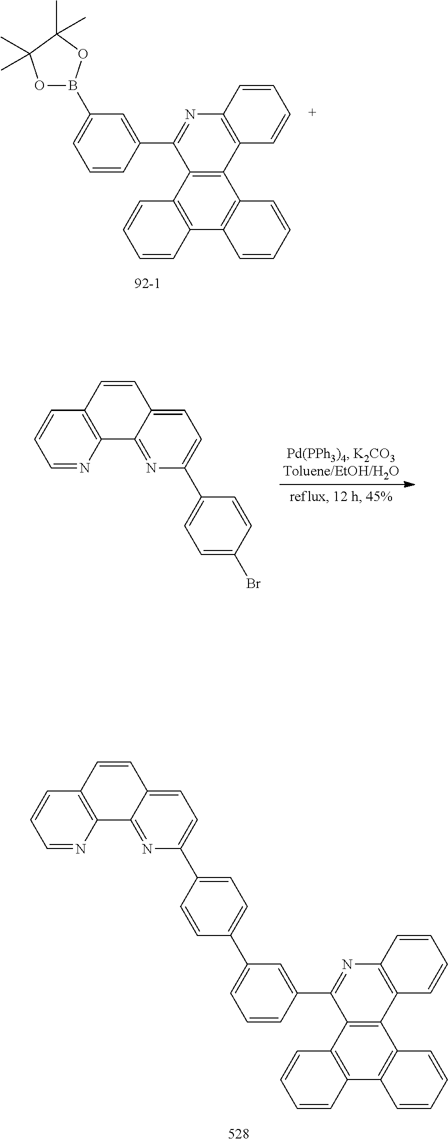

Preparation of Compound 528

A mixed solution of Compound 92-1 (10.0 g, 0.0208 mol), 2-(4-bromophenyl)-1, 10-phenanthroline (6.94 g, 0.0249 mol), K₂CO₃ (8.61 g, 0.062 mol), Pd(PPh₃)₄ (1.20 g, 0.00104 mol), and toluene, ethanol, H₂O (200 ml/30 ml/30 ml) was refluxed and stirred in a two neck round bottom flask (two neck r.b.f) under nitrogen for 12 hours. A precipitated solid was filtered with the reaction mixture cooled to room temperature and washed with toluene and methanol. The filtered solid was dissolved in an excessive amount of hot toluene and allowed to pass through silica gel and then concentrated. The resultant reaction product was dissolved in a small amount of EA and in a small amount of 1,2-dichloroethane and then filtered. As a result, a white solid compound 528 (5.70 g, 45%) was obtained.

### Preparation Example 26

### Preparation of Compound 530

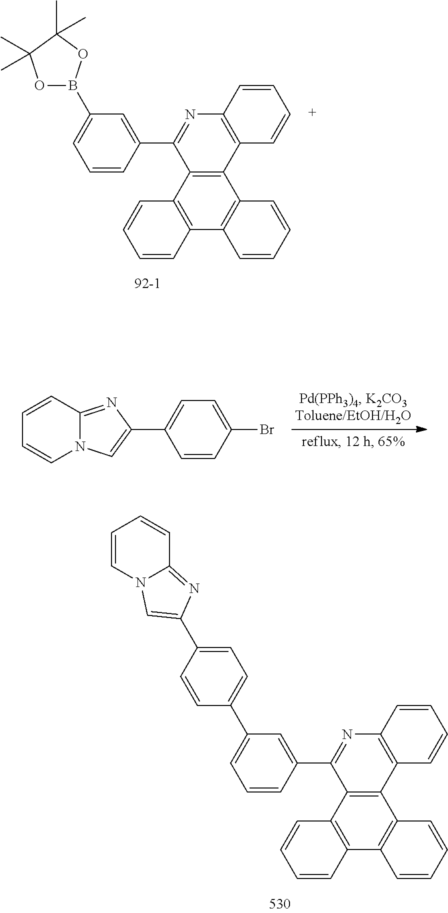

Preparation of Compound 530

A mixed solution of Compound 92-1 (10.0 g, 0.0208 mol), 2-(4-bromophenyl)imidazo[1,2-a]pyridine (6.80 g, 0.0249 mol), K₂CO₃ (8.61 g, 0.062 mol), Pd(PPh₃)₄ (1.20 g, 0.0104 mol), and toluene, ethanol, H₂O (200 ml/30 ml/30 ml) was refluxed and stirred in a two neck round bottom flask (two neck r.b.f) under nitrogen for 12 hours. The reaction mixture cooled to room temperature was extracted with MC (methylene chloride)/H₂O and dried using MgSO₄ and then filtered. After being concentrated, the resultant reaction product was separated by column chromatography (SiO₂, hexane:MC=2:1). As a result, a white solid compound 530 (7.40 g, 65%) was obtained.

### Preparation Example 27

### Preparation of Compound 538

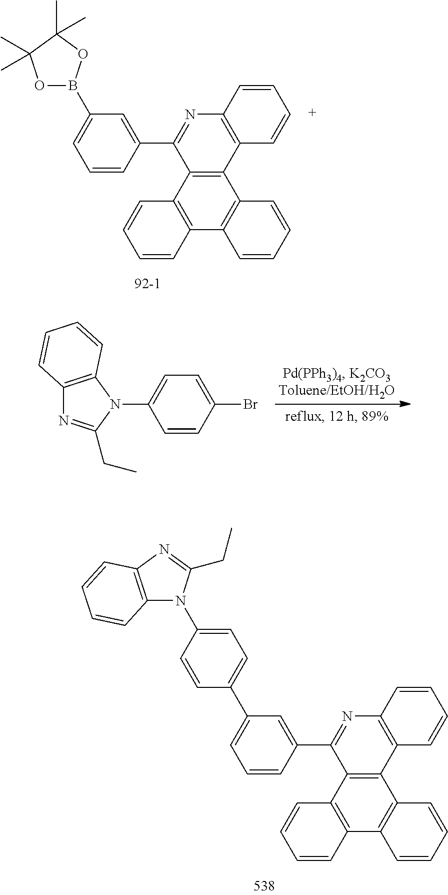

Preparation of Compound 538

A mixed solution of Compound 92-1 (10.0 g, 0.0208 mol), 1-(4-bromophenyl)-2-ethyl-1H-benzo[d]imidazole (7.50 g, 0.0249 mol), K₂CO₃ (8.61 g, 0.062 mol), Pd(PPh₃)₄ (1.20 g, 0.00104 mol), and toluene, ethanol, H₂O (200 ml/30 ml/30 ml) was refluxed and stirred in a two neck round bottom flask (two neck r.b.f) under nitrogen for 12 hours. The reaction mixture cooled to room temperature was extracted with MC (methylene chloride)/H₂O and dried using MgSO₄ and then filtered. After being concentrated, the resultant reaction product was separated by column chromatography (SiO₂, hexane:MC=2:1). As a result, a white solid compound 538 (10.66 g, 89%) was obtained.

### Preparation Example 28

### Preparation of Compound 558

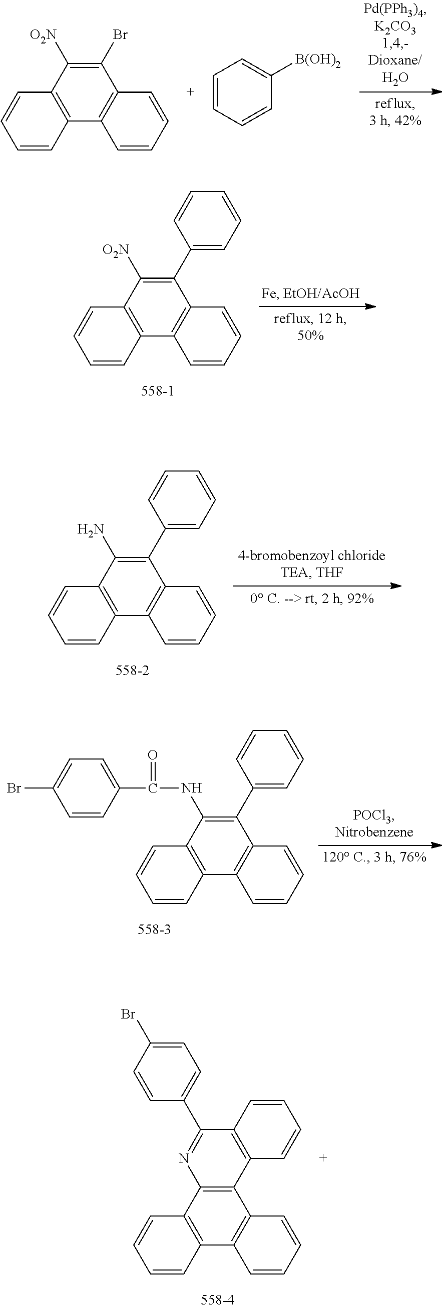

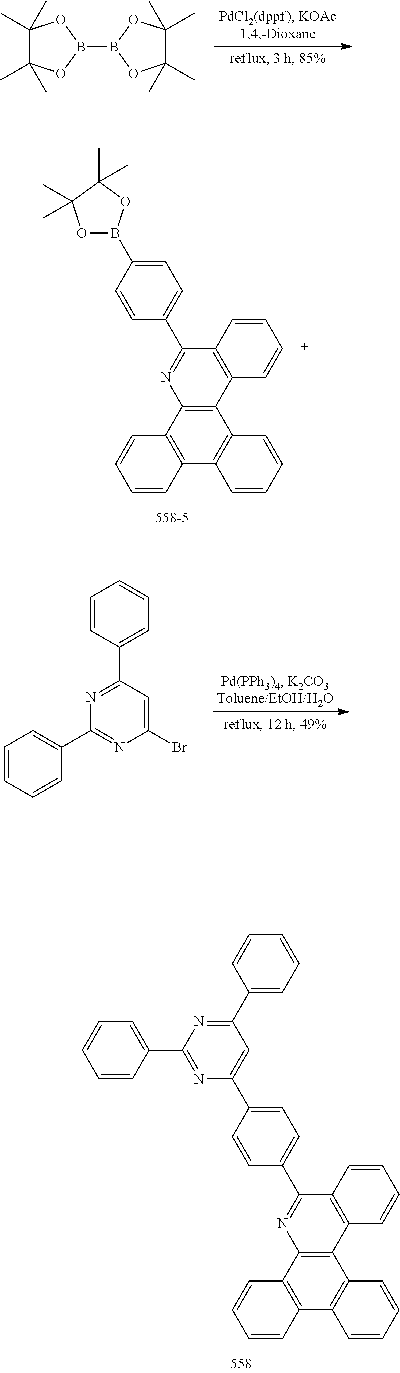

Preparation of Compound 558-1

A mixed solution of 9-bromo-10-nitrophenanthrene (100 g, 0.33 mol), phenyl boronic acid (60.3 g, 0.495 mol), K₃PO₄ (251.4 g, 0.99 mmol), Pd(PPh₃)₄ (19.0 g, 0.0165 mol), and 1,4-dioxane)/H₂O (2000 ml/400 ml) was refluxed and stirred in a two neck round bottom flask (two neck r.b.f) under nitrogen for 3 hours. The reaction mixture cooled to room temperature was extracted with MC (methylene chloride)/H₂O and dried using MgSO₄ and then filtered. After being concentrated, the resultant reaction product was separated by column chromatography (SiO₂, hexane:MC=2:1). As a result, a solid compound 558-1 (41.5 g, 42%) was obtained.

Preparation of Compound 558-2

Compound 558-1 (40 g, 0.133 mol), iron (Fe) (8.17 g, 0.146 mol), 150 ml of acetic acid, and 800 ml of ethanol were mixed and then refluxed and stirred in a two neck round bottom flask (two neck r.b.f) for 12 hours. The reaction mixture cooled to room temperature was extracted with MC (methylene chloride)/H₂O and dried using MgSO₄ and then filtered. After being concentrated, the resultant reaction product was separated by column chromatography (SiO₂, hexane:MC=2:1). As a result, a solid compound 558-2 (18 g, 50%) was obtained.

Preparation of Compound 558-3

Compound 558-2 (18 g, 0.0668 mol) was dissolved in THF (100 ml) within a one neck round bottom flask (one neck r.b.f), and triethylamine (27.9 ml, 0.20 mol) was added dropwise thereto at room temperature. After the reaction mixture was cooled to 0° C., 4-bromobenzoyl chloride (16.1 g, 0.0735 mol) was dissolved in 25 ml of THF and slowly added dropwise thereto at a low temperature. Then, the resultant reaction product was stirred at room temperature for 2 hours and added with H₂O to terminate the reaction. The solvent was distilled under reduced pressure, and then MC (methylene chloride)/H₂O was extracted. The resultant reaction product was dried using MgSO₄ and then filtered and concentrated. After the resultant reaction product was dissolved in a small amount of MC (methylene chloride), methanol was added to precipitate a solid. As a result, an ivory solid compound 558-3 (27.8 g, 92%) was obtained.

Preparation of Compound 558-4

Compound 558-3 (27 g, 0.0597 mol) was dissolved in nitrobenzene (150 ml) within a one neck round bottom flask (one neck r.b.f), and POCl₃ (phosphorous oxychloride) (3.33 ml, 0.0358 mmol) was added dropwise thereto at room temperature and then refluxed and stirred for 12 hours. After the reaction was terminated, the mixed solution was cooled to room temperature and neutralized with NaHCO₃ aqueous solution. Then, MC (methylene chloride)/H₂O was extracted. The resultant reaction product was dried using MgSO₄ and then filtered. After the solvent was distilled under reduced pressure and concentrated, a small amount of MC (methylene chloride) and an excessive amount of hexane were added to precipitate a solid. As a result, a pale yellow solid compound 558-4 (19.7 g, 76%) was obtained.

Preparation of Compound 558-5

A mixed solution of Compound 558-4 (15 g, 0.0345 mol), bis(pinacolato) diboron (13.1 g, 0.0518 mol), KOAc (potassium acetate) (10.2 g, 0.104 mol), PdCl₂(dppf) (1.58 g, 0.00173 mol), and 250 ml of 1,4-dioxane was put into a two neck round bottom flask (two neck r.b.f) and refluxed and stirred therein under nitrogen for 3 hours. A reaction mixture cooled to room temperature was extracted with MC (methylene chloride)/H₂O and dried using MgSO₄ and then filtered. After being concentrated, a solid is produced with hexane and filtered. As a result, a solid compound 558-5 (14.1 g, 85%) was obtained.

Preparation of Compound 558

A mixed solution of Compound 558-5 (10.0 g, 0.0208 mol), 4-bromo-2,6-diphenylpyrimidine (7.75 g, 0.0249 mol), K₂CO₃ (8.61 g, 0.062 mol), Pd(PPh₃)₄ (1.20 g, 0.00104 mol), and toluene, ethanol, H₂O (200 ml/30 ml/30 ml) was refluxed and stirred in a two neck round bottom flask (two neck r.b.f) under nitrogen for 12 hours. The reaction mixture cooled to room temperature was extracted with MC (methylene chloride)/H₂O and dried using MgSO₄ and then allowed to pass through silica gel. After being concentrated, the resultant reaction product was dissolved in a small amount of EA, in a small amount of 1, 2-dichloroethane, and in a small amount of methanol and stirred for 3 or more hours and then filtered. As a result, a white compound 558 (9.99 g, 82%) was obtained.

### Preparation Example 29

### Preparation of Compound 559

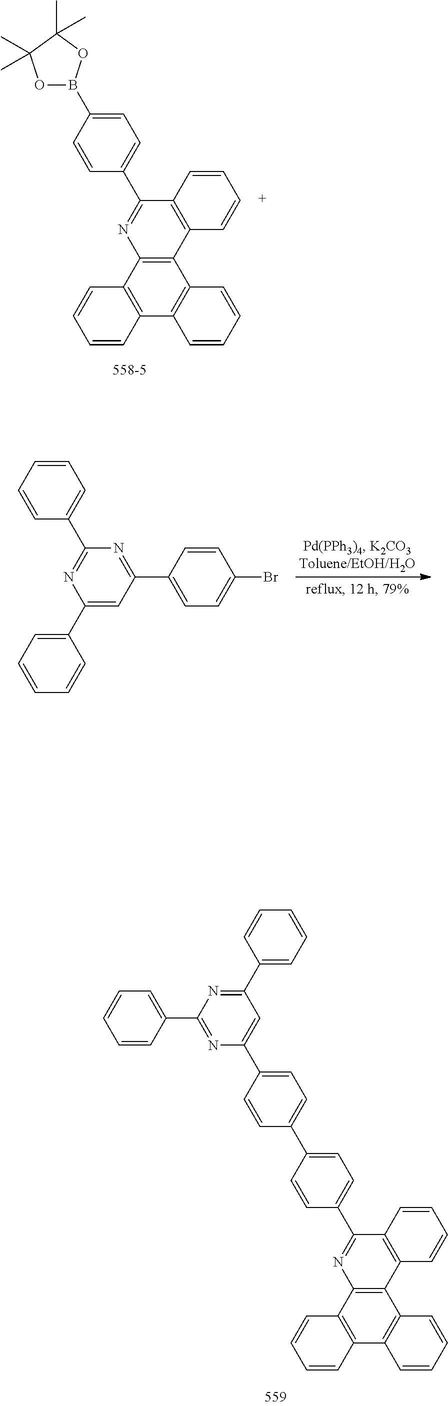

Preparation of Compound 559

A mixed solution of Compound 558-5 (10.0 g, 0.0208 mol), 4-(4-bromophenyl)-2, 6-diphenylpyrimidine (9.64 g, 0.0249 mol), K₂CO₃ (8.61 g, 0.062 mol), Pd(PPh₃)₄ (1.20 g, 0.00104 mol), and toluene, ethanol, H₂O (200 ml/30 ml/30 ml) was refluxed and stirred in a two neck round bottom flask (two neck r.b.f) under nitrogen for 12 hours. A precipitated solid was filtered with the reaction mixture cooled to room temperature and washed with toluene and methanol. The filtered solid was dissolved in an excessive amount of hot toluene and allowed to pass through silica gel and then concentrated. The resultant reaction product was dissolved in a small amount of EA and in a small amount of 1,2-dichloroethane and then filtered. As a result, a white solid compound 559 (10.87 g, 79%) was obtained.

### Preparation Example 30

### Preparation of Compound 560

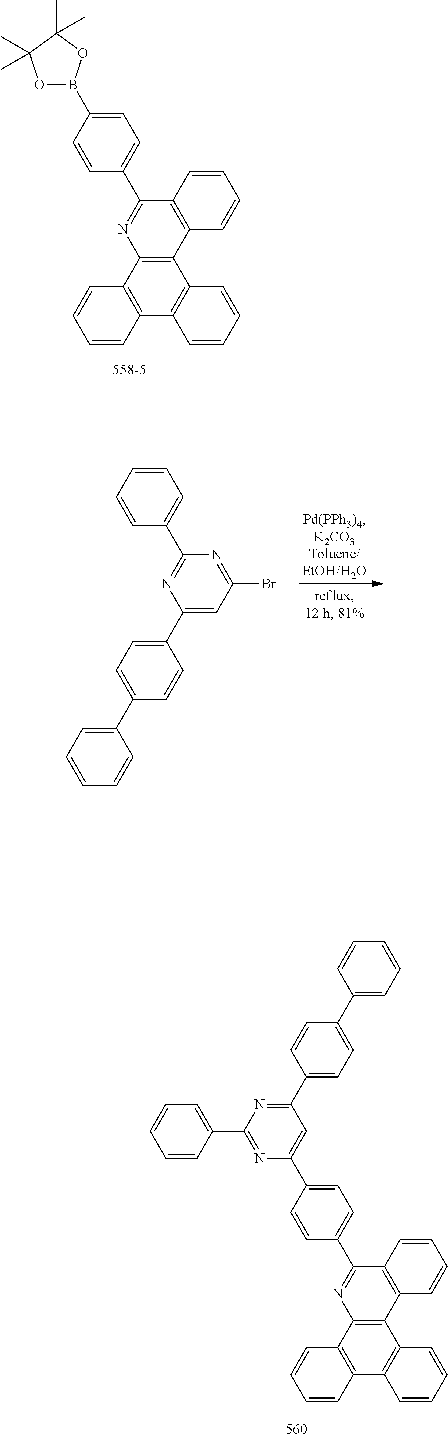

Preparation of Compound 560

A mixed solution of Compound 558-5 (10.0 g, 0.0208 mol), 4-([1,1′-biphenyl]-4-yl)-6-bromo-2-phenylpyrimidine (9.64 g, 0.0249 mol), K₂CO₃ (8.61 g, 0.062 mol), Pd(PPh₃)₄ (1.20 g, 0.00104 mol), and toluene, ethanol, H₂O (200 ml/30 ml/30 ml) was refluxed and stirred in a two neck round bottom flask (two neck r.b.f) under nitrogen for 12 hours. A precipitated solid was filtered with the reaction mixture cooled to room temperature and washed with toluene and methanol. The filtered solid was dissolved in an excessive amount of hot toluene and allowed to pass through silica gel and then concentrated. The resultant reaction product was dissolved in a small amount of EA and in a small amount of 1,2-dichloroethane and then filtered. As a result, a white solid compound 560 (11.15 g, 81%) was obtained.

### Preparation Example 31

### Preparation of Compound 561

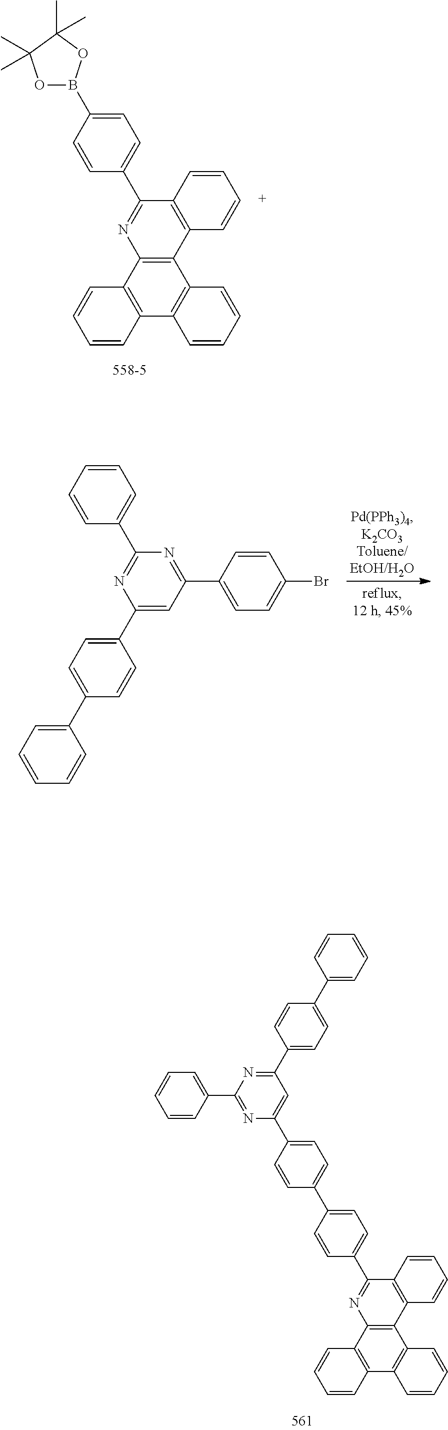

Preparation of Compound 561

A mixed solution of Compound 558-5 (10.0 g, 0.0208 mol), 4-([1,1′-biphenyl]-4-yl)-6-(4-bromophenyl)-2-phenylpyrimidine (11.54 g, 0.0249 mol), K₂CO₃ (8.61 g, 0.062 mol), Pd(PPh₃)₄ (1.20 g, 0.00104 mol), and toluene, ethanol, H₂O (200 ml/30 ml/30 ml) was refluxed and stirred in a two neck round bottom flask (two neck r.b.f) under nitrogen for 12 hours. A precipitated solid was filtered with the reaction mixture cooled to room temperature and washed with toluene and methanol. The filtered solid was dissolved in an excessive amount of hot toluene and allowed to pass through silica gel and then concentrated. The resultant reaction product was dissolved in a small amount of EA and in a small amount of 1,2-dichloroethane and then filtered. As a result, a white solid compound 561 (6.90 g, 45%) was obtained.

### Preparation Example 32

### Preparation of Compound 562

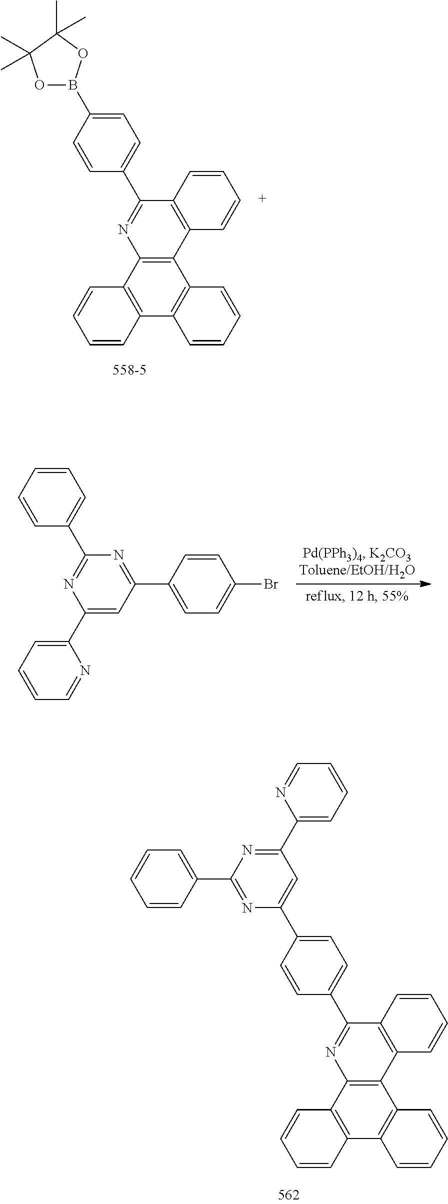

Preparation of Compound 562

A mixed solution of Compound 558-5 (10.0 g, 0.0208 mol), 4-bromo-2-phenyl-6-(pyridin-2-yl)pyrimidine (7.77 g, 0.0249 mol), K₂CO₃ (8.62 g, 0.062 mol), Pd(PPh₃)₄ (1.20 g, 0.0104 mol), and toluene, ethanol, H₂O (200 ml/30 ml/30 ml) was refluxed and stirred in a two neck round bottom flask (two neck r.b.f) under nitrogen for 12 hours. The reaction mixture cooled to room temperature was extracted with MC (methylene chloride)/H₂O and dried using MgSO₄ and then filtered. After being concentrated, the resultant reaction product was separated by column chromatography (SiO₂, hexane:MC=2:1). As a result, a white solid compound 562 (6.71 g, 55%) was obtained.

### Preparation Example 33

### Preparation of Compound 563

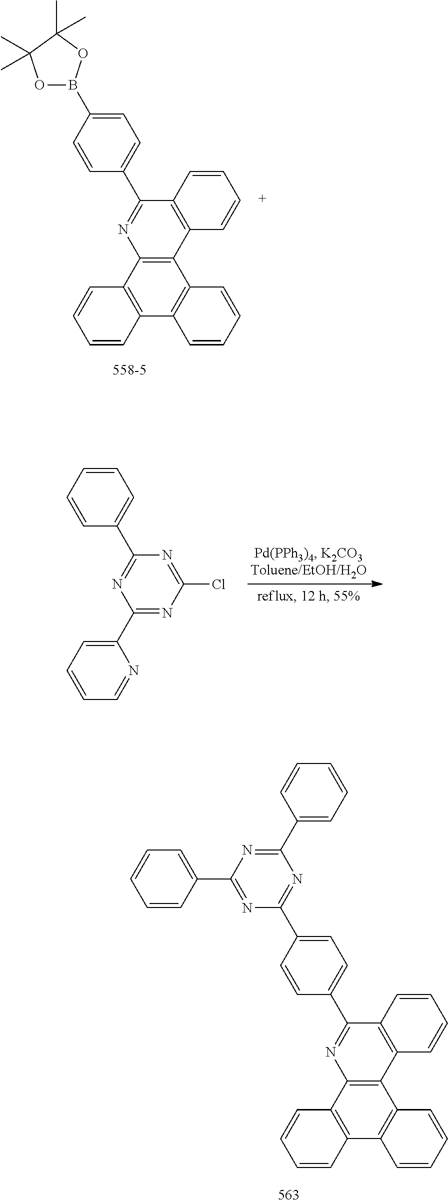

Preparation of Compound 563

A mixed solution of Compound 558-5 (10.0 g, 0.0208 mol), 2-chloro-4,6-diphenyl-1,3,5-triazine (6.66 g, 0.0249 mol), K₂CO₃ (8.61 g, 0.062 mol), Pd(PPh₃)₄ (1.20 g, 0.00104 mol), and toluene, ethanol, H₂O (200 ml/30 ml/30 ml) was refluxed and stirred in a two neck round bottom flask (two neck r.b.f) under nitrogen for 12 hours. A precipitated solid was filtered with the reaction mixture cooled to room temperature and washed with toluene and methanol. The filtered solid was dissolved in an excessive amount of hot toluene and allowed to pass through silica gel and then concentrated. The resultant reaction product was dissolved in a small amount of EA and in a small amount of 1,2-dichloroethane and then filtered. As a result, a white solid compound 563 (6.95 g, 57%) was obtained.

### Preparation Example 34

### Preparation of Compound 564

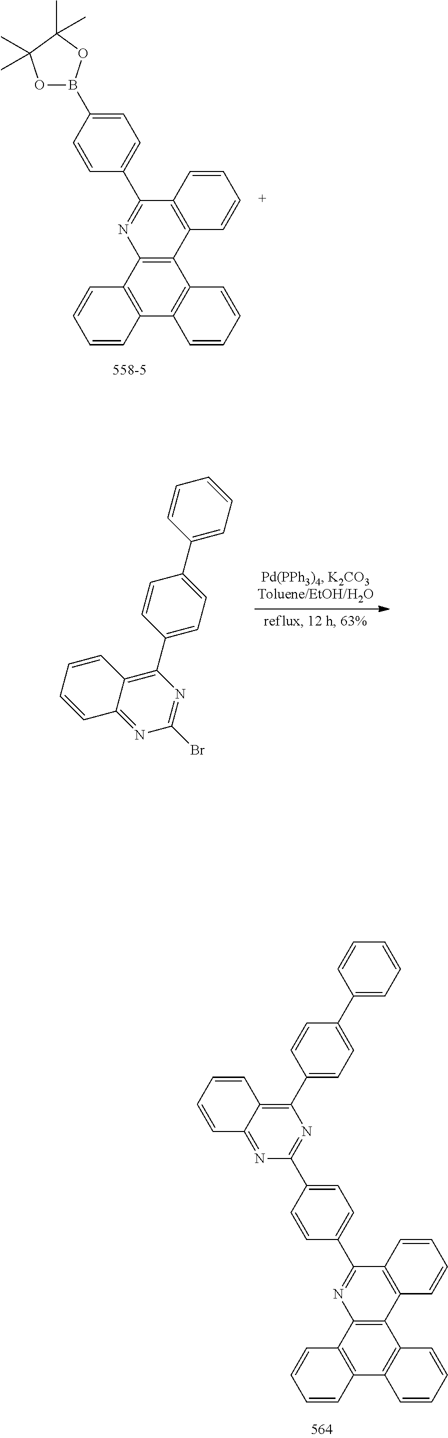

Preparation of Compound 564

A mixed solution of Compound 558-5 (10.0 g, 0.0208 mol), 4-([1,1′-biphenyl]-4-yl)-2-bromoquinazoline (8.99 g, 0.0249 mol), K₂CO₃ (8.61 g, 0.062 mol), Pd(PPh₃)₄ (1.20 g, 0.00104 mol), and toluene, ethanol, H₂O (200 ml/30 ml/30 ml) was refluxed and stirred in a two neck round bottom flask (two neck r.b.f) under nitrogen for 12 hours. The reaction mixture cooled to room temperature was extracted with MC (methylene chloride)/H₂O and dried using MgSO₄ and then allowed to pass through silica gel. After being concentrated, the resultant reaction product was dissolved in a small amount of EA, in a small amount of 1,2-dichloroethane, and in a small amount of methanol and stirred for 3 or more hours and then filtered. As a result, a white compound 564 (8.33 g, 63%) was obtained.

### Preparation Example 35

### Preparation of Compound 565

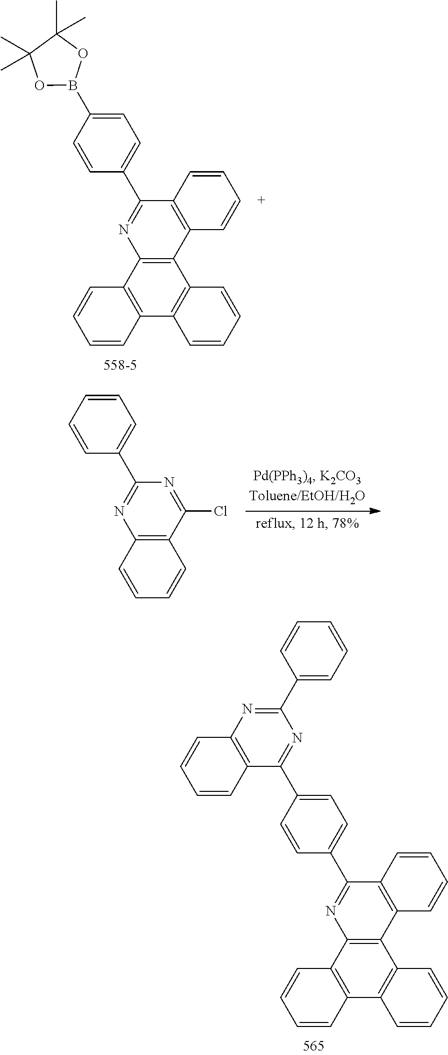 [SMILES: CC1(C)OB(c2ccc(-c3nc4c5ccccc5c5ccccc5c4c4ccccc34)cc2)OC1(C)C ; Clc1nc(-c2ccccc2)nc2ccccc12 ; c1ccc(-c2nc(-c3ccc(-c4nc5c6ccccc6c6ccccc6c5c5ccccc45)cc3)c3ccccc3n2)cc1]

Preparation of Compound 565

A mixed solution of Compound 558-5 (10.0 g, 0.0208 mol), 4-chloro-2-phenylquinazoline (5.99 g, 0.0249 mol), K₂CO₃ (8.61 g, 0.062 mol), Pd(PPh₃)₄ (1.20 g, 0.00104 mol), and toluene, ethanol, H₂O (200 ml/30 ml/30 ml) was refluxed and stirred in a two neck round bottom flask (two neck r.b.f) under nitrogen for 12 hours. The reaction mixture cooled to room temperature was extracted with MC (methylene chloride)/H₂O and dried using MgSO₄ and then allowed to pass through silica gel. After being concentrated, the resultant reaction product was dissolved in a small amount of EA, in a small amount of 1, 2-dichloroethane, and in a small amount of methanol and stirred for 3 or more hours and then, filtered. As a result, a white compound 565 (9.08 g, 78%) was obtained.

### Preparation Example 36

### Preparation of Compound 566

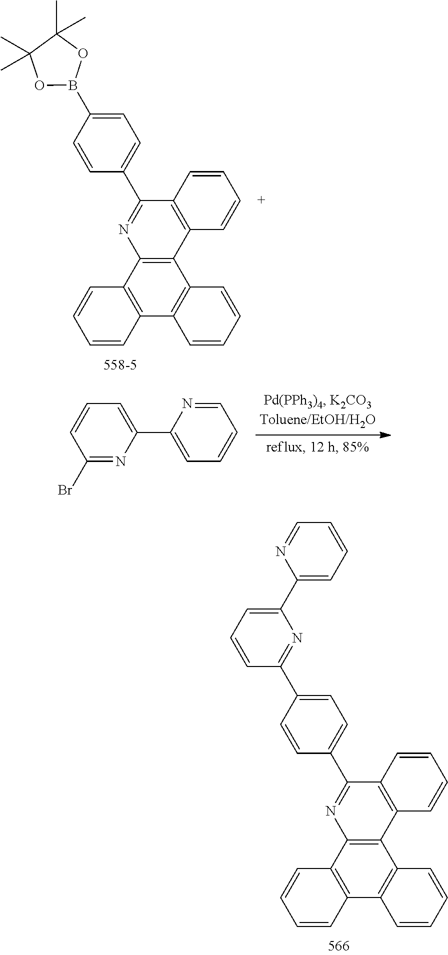 [SMILES: CC1(C)OB(c2ccc(-c3nc4c5ccccc5c5ccccc5c4c4ccccc34)cc2)OC1(C)C ; Brc1cccc(-c2ccccn2)n1 ; c1ccc(-c2cccc(-c3ccc(-c4nc5c6ccccc6c6ccccc6c5c5ccccc45)cc3)n2)nc1]

Preparation of Compound 566

A mixed solution of Compound 558-5 (10.0 g, 0.0208 mol), 6-bromo-2, 2′-bipyridine (5.85 g, 0.0249 mol), K₂CO₃ (8.61 g, 0.062 mol), Pd(PPh₃)₄ (1.20 g, 0.00104 mol), and toluene, ethanol, H₂O (200 ml/30 ml/30 ml) was refluxed and stirred in a two neck round bottom flask (two neck r.b.f) under nitrogen for 12 hours. The reaction mixture cooled to room temperature was extracted with MC (methylene chloride)/H₂O and dried using MgSO₄ and then filtered. After being concentrated, the resultant reaction product was separated by column chromatography (SiO₂, hexane:MC=2:1). As a result, a white solid compound 566 (9.0 g, 85%) was obtained.

### Preparation Example 37

### Preparation of Compound 567

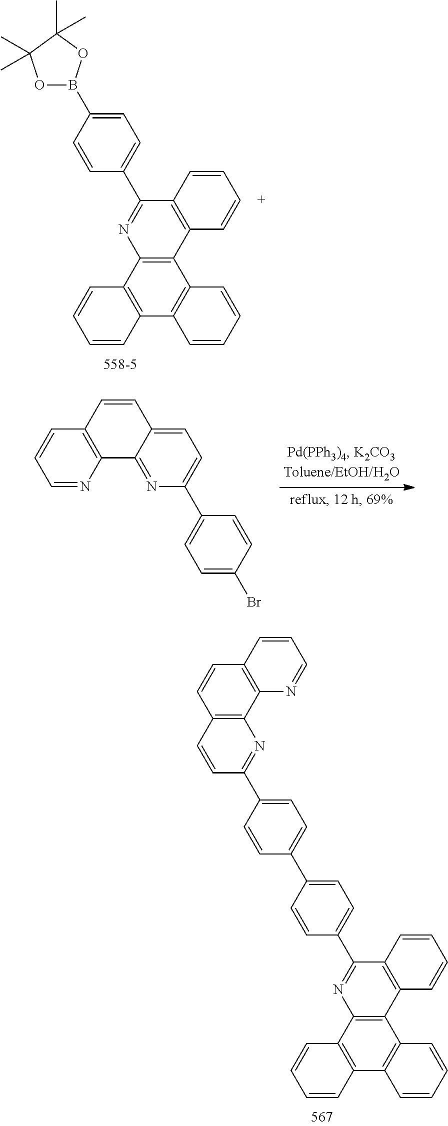

Preparation of Compound 567

A mixed solution of Compound 558-5 (10.0 g, 0.0208 mol), 2-(4-bromophenyl)-1, 10-phenanthroline (8.35 g, 0.0249 mol), K₂CO₃ (8.61 g, 0.062 mol), Pd(PPh₃)₄ (1.20 g, 0.00104 mol), and toluene, ethanol, H₂O (200 ml/30 ml/30 ml) was refluxed and stirred in a two neck round bottom flask (two neck r.b.f) under nitrogen for 12 hours. A precipitated solid was filtered with the reaction mixture cooled to room temperature and washed with toluene and methanol. The filtered solid was dissolved in an excessive amount of hot toluene and allowed to pass through silica gel and then concentrated. The resultant reaction product was dissolved in a small amount of EA and in a small amount of 1,2-dichloroethane and then filtered. As a result, a white solid compound 567 (8.75 g, 69%) was obtained.

### Preparation Example 38

### Preparation of Compound 568

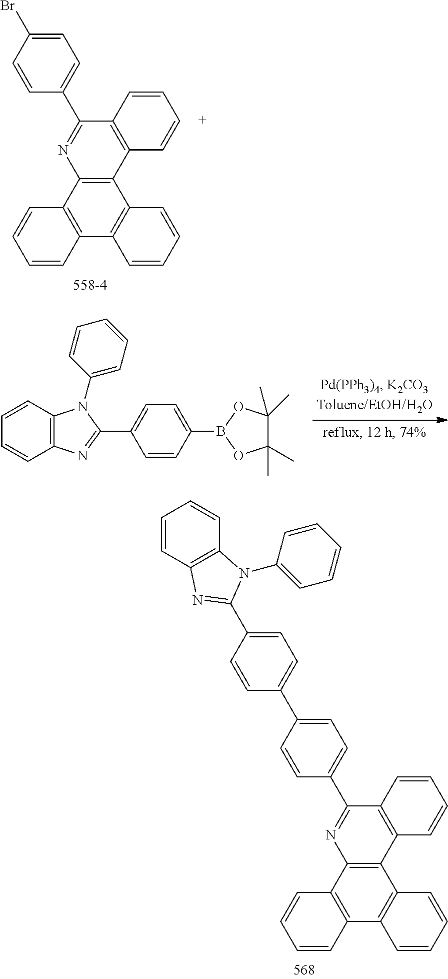

Preparation of Compound 568

A mixed solution of Compound 558-4 (10.0 g, 0.023 mol), 1-phenyl-2-(4-(4,4,5,5-tetramethyl-1,3,2-dioxaborolan-2-yl)phenyl)-1H-benzo[d]imidazole (10.95 g, 0.0276 mol), K₂CO₃ (9.52 g, 0.069 mmol), Pd(PPh₃)₄ (1.33 g, 0.00115 mol), and toluene, ethanol, H₂O (200 ml/30 ml/30 ml) was refluxed and stirred in a two neck round bottom flask (two neck r.b.f) under nitrogen for 12 hours. The reaction mixture cooled to room temperature was extracted with MC (methylene chloride)/H₂O and dried using MgSO₄ and then allowed to pass through silica gel. After being concentrated, the resultant reaction product was dissolved in a small amount of EA, in a small amount of 1,2-dichloroethane, and in a small amount of methanol and stirred for 3 or more hours and then filtered. As a result, a white compound 568 (9.60 g, 74%) was obtained.

### Preparation Example 39

### Preparation of Compound 569

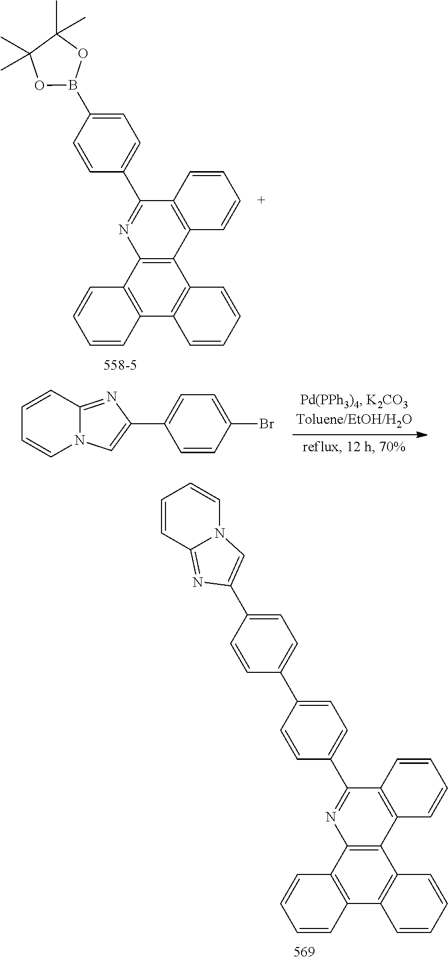

Preparation of Compound 569

A mixed solution of Compound 558-5 (10.0 g, 0.0208 mol), 2-(4-bromophenyl)imidazo[1,2-a]pyridine (6.80 g, 0.0249 mol), K₂CO₃ (8.61 g, 0.062 mol), Pd(PPh₃)₄ (1.20 g, 0.00104 mol), and toluene, ethanol, H₂O (200 ml/30 ml/30 ml) was refluxed and stirred in a two neck round bottom flask (two neck r.b.f) under nitrogen for 12 hours. The reaction mixture cooled to room temperature was extracted with MC (methylene chloride)/H₂O and dried using MgSO₄ and then allowed to pass through silica gel. After being concentrated, the resultant reaction product was dissolved in a small amount of EA, in a small amount of 1,2-dichloroethane, and in a small amount of methanol and stirred for 3 or more hours and then filtered. As a result, a white compound 569 (7.97 g, 70%) was obtained.

### Preparation Example 40

### Preparation of Compound 570

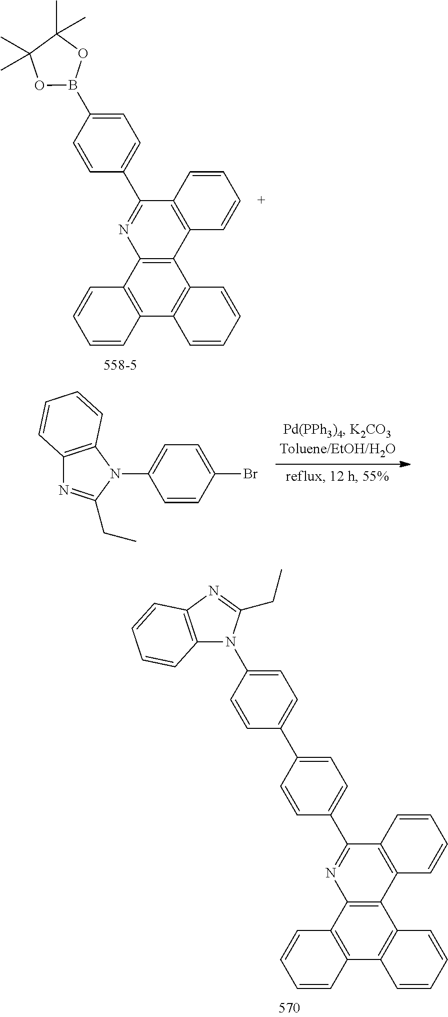 [SMILES: CC1(C)OB(c2ccc(-c3nc4c5ccccc5c5ccccc5c4c4ccccc34)cc2)OC1(C)C ; CCc1nc2ccccc2n1-c1ccc(Br)cc1 ; CCc1nc2ccccc2n1-c1ccc(-c2ccc(-c3nc4c5ccccc5c5ccccc5c4c4ccccc34)cc2)cc1]

Preparation of Compound 570

A mixed solution of Compound 558-5 (10.0 g, 0.0208 mol), 1-(4-bromophenyl)-2-ethyl-1H-benzo[d]imidazole (7.50 g, 0.0249 mol), K₂CO₃ (8.61 g, 0.062 mol), Pd(PPh₃)₄ (1.20 g, 0.00104 mol), and toluene, ethanol, H₂O (200 ml/30 ml/30 ml) was refluxed and stirred in a two neck round bottom flask (two neck r.b.f) under nitrogen for 12 hours. The reaction mixture cooled to room temperature was extracted with MC (methylene chloride)/H₂O and dried using MgSO₄ and then filtered. After being concentrated, the resultant reaction product was separated by column chromatography (SiO₂, hexane:MC=2:1). As a result, a white solid compound 570 (6.59 g, 55%) was obtained.

### Preparation Example 41

### Preparation of Compound 571

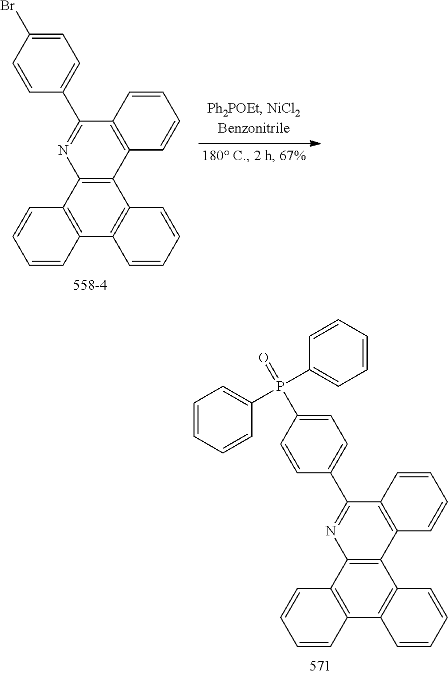 [SMILES: Brc1ccc(-c2nc3c4ccccc4c4ccccc4c3c3ccccc23)cc1 ; O=P(c1ccccc1)(c1ccccc1)c1ccc(-c2nc3c4ccccc4c4ccccc4c3c3ccccc23)cc1]

Preparation of Compound 571

A mixed solution of Compound 558-4 (10.0 g, 0.023 mol), nickel(II) chloride (1.79 g, 0.0138 mol), and benzonitrile was heated to 180° C. and stirred for 1 hour in a two neck round bottom flask (two neck r.b.f) under nitrogen. Ph₂POEt (ethyl diphenylphosphinite) (8.48 g, 0.0368 mol) was slowly added dropwise to the above mixture and then stirred for 2 hours. The reaction mixture cooled to room temperature was extracted with MC (methylene chloride)/H₂O and dried using MgSO₄ and then filtered. After being concentrated, the resultant reaction product was separated by column chromatography (SiO₂, hexane:MC=2:1). As a result, a white solid compound 571 (6.4 g, 58%) was obtained.

### Preparation Example 42

### Preparation of Compound 572

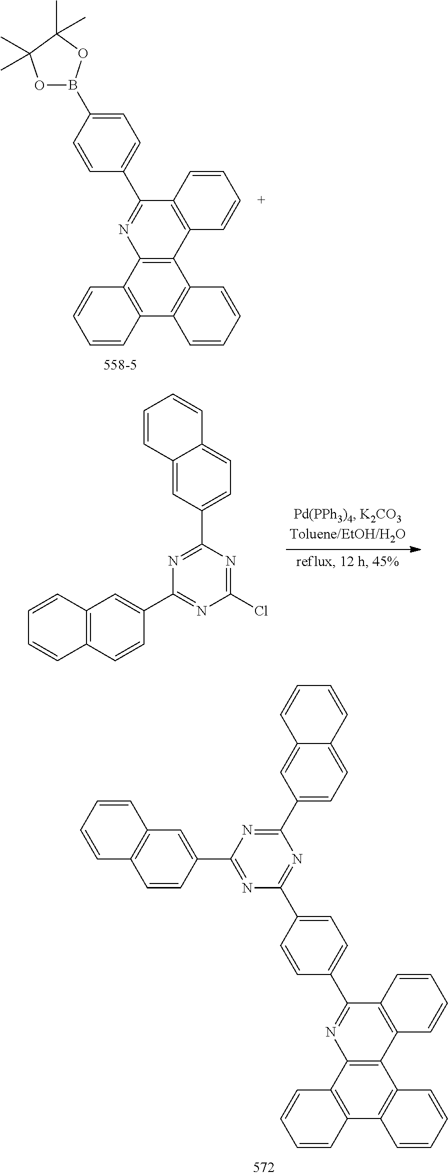 [SMILES: CC1(C)OB(c2ccc(-c3nc4c5ccccc5c5ccccc5c4c4ccccc34)cc2)OC1(C)C ; Clc1nc(-c2ccc3ccccc3c2)nc(-c2ccc3ccccc3c2)n1 ; c1ccc2cc(-c3nc(-c4ccc(-c5nc6c7ccccc7c7ccccc7c6c6ccccc56)cc4)nc(-c4ccc5ccccc5c4)n3)ccc2c1]

Preparation of Compound 572

A mixed solution of Compound 558-5 (10.0 g, 0.0208 mol), 2-chloro-4,6-di(naphthalen-2-yl)-1,3,5-triazine (9.16 g, 0.0249 mol), K₂CO₃ (8.61 g, 0.062 mol), Pd(PPh₃)₄ (1.20 g, 0.00104 mol), and toluene, ethanol, H₂O (200 ml/30 ml/30 ml) was refluxed and stirred in a two neck round bottom flask (two neck r.b.f) under nitrogen for 12 hours. A precipitated solid was filtered with the reaction mixture cooled to room temperature and washed with toluene and methanol. The filtered solid was dissolved in an excessive amount of hot toluene and allowed to pass through silica gel and then concentrated. The resultant reaction product was dissolved in a small amount of EA and in a small amount of 1,2-dichloroethane and then filtered. As a result, a white solid compound 572 (6.43 g, 45%) was obtained.

### Preparation Example 43

### Preparation of Compound 577

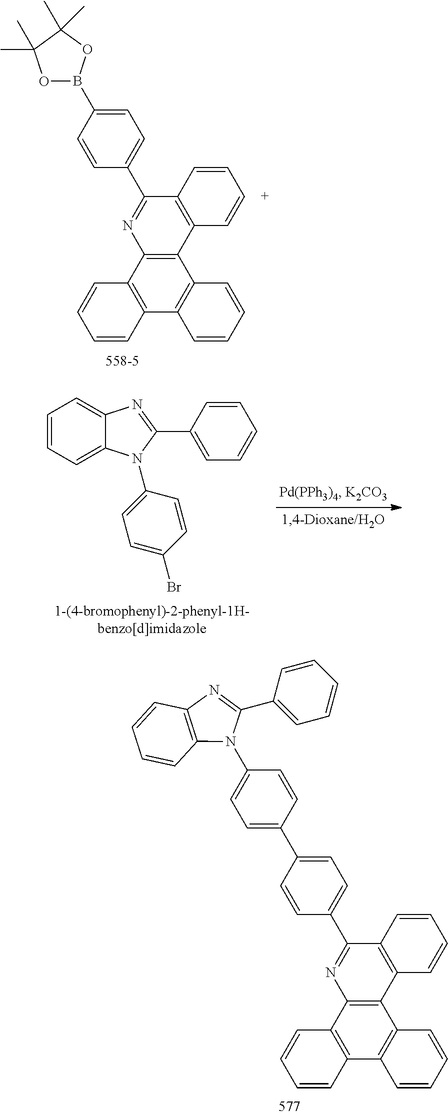 [SMILES: CC1(C)OB(c2ccc(-c3nc4c5ccccc5c5ccccc5c4c4ccccc34)cc2)OC1(C)C ; Brc1ccc(-n2c(-c3ccccc3)nc3ccccc32)cc1 ; c1ccc(-c2nc3ccccc3n2-c2ccc(-c3ccc(-c4nc5c6ccccc6c6ccccc6c5c5ccccc45)cc3)cc2)cc1]

Preparation of Compound 577

The title compound was prepared in the same manner as in the preparation method of Compound 558, except that 1-(4-bromophenyl)-2-phenyl-1H-benzo[d]imidazole was used instead of 4-bromo-2,6-diphenylpyrimidine (10.2 g, 79%).

### Preparation Example 44

### Preparation of Compound 590

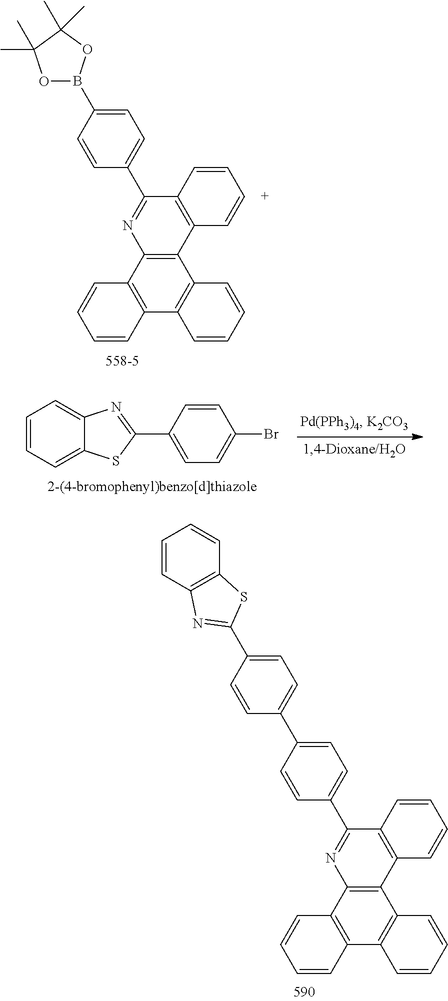 [SMILES: CC1(C)OB(c2ccc(-c3nc4c5ccccc5c5ccccc5c4c4ccccc34)cc2)OC1(C)C ; Brc1ccc(-c2nc3ccccc3s2)cc1 ; c1ccc2sc(-c3ccc(-c4ccc(-c5nc6c7ccccc7c7ccccc7c6c6ccccc56)cc4)cc3)nc2c1]

Preparation of Compound 590

The title compound was prepared in the same manner as in the preparation method of Compound 558, except that 2-(4-bromophenyl)benzo[d]thiazole was used instead of 4-bromo-2,6-diphenylpyrimidine (6.1 g, 52%).

### Preparation Example 45

### Preparation of Compound 597

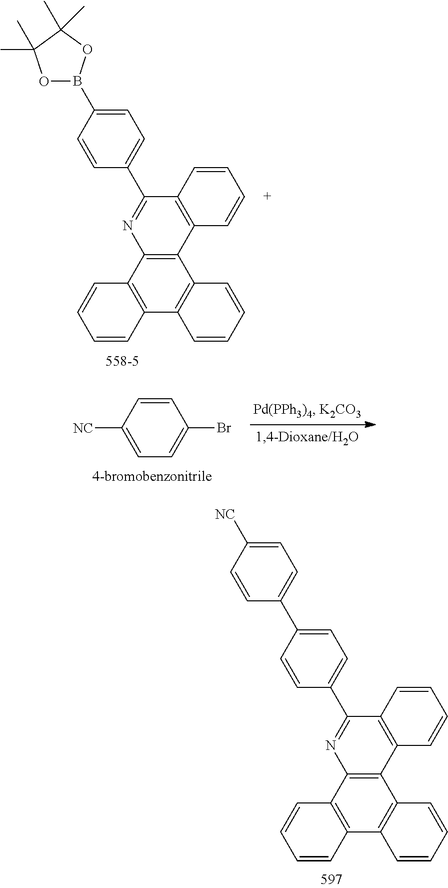 [SMILES: CC1(C)OB(c2ccc(-c3nc4c5ccccc5c5ccccc5c4c4ccccc34)cc2)OC1(C)C ; N#Cc1ccc(Br)cc1 ; N#Cc1ccc(-c2ccc(-c3nc4c5ccccc5c5ccccc5c4c4ccccc34)cc2)cc1]

Preparation of Compound 597

The title compound was prepared in the same manner as in the preparation method of Compound 558, except that 4-bromobenzonitrile was used instead of 4-bromo-2,6-diphenylpyrimidine (6.26 g, 66%).

### Preparation Example 46

### Preparation of Compound 598

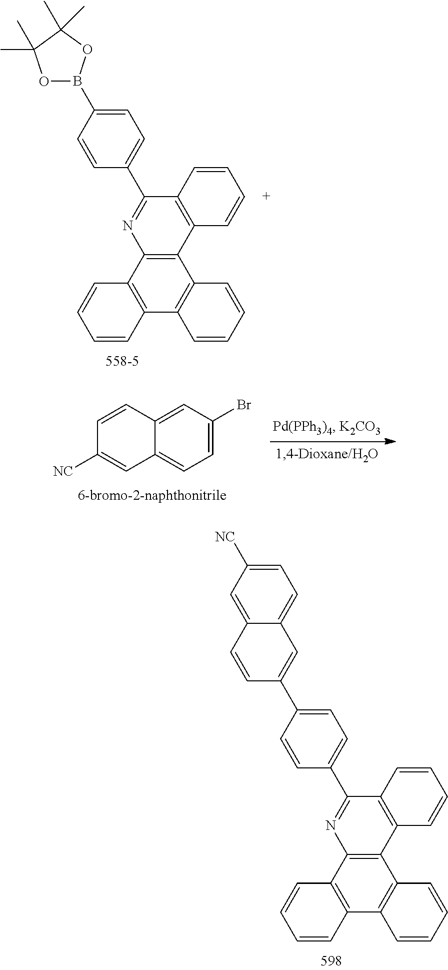 [SMILES: CC1(C)OB(c2ccc(-c3nc4c5ccccc5c5ccccc5c4c4ccccc34)cc2)OC1(C)C ; N#Cc1ccc2cc(Br)ccc2c1 ; N#Cc1ccc2cc(-c3ccc(-c4nc5c6ccccc6c6ccccc6c5c5ccccc45)cc3)ccc2c1]

Preparation of Compound 598

The title compound was prepared in the same manner as in the preparation method of Compound 558, except that 4-bromobenzonitrile was used instead of 4-bromo-2,6-diphenylpyrimidine (5.68 g, 54%).

### Preparation Example 47

### Preparation of Compound 599

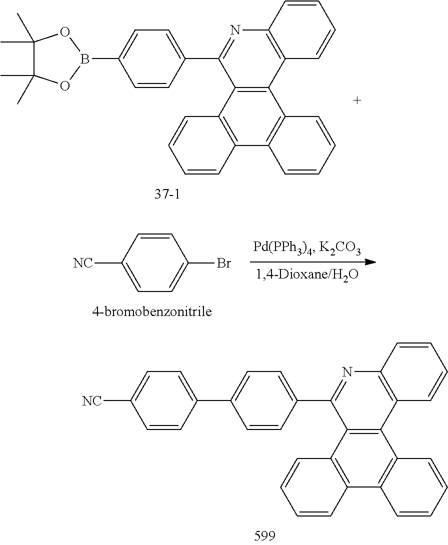 [SMILES: CC1(C)OB(c2ccc(-c3nc4ccccc4c4c5ccccc5c5ccccc5c34)cc2)OC1(C)C ; N#Cc1ccc(Br)cc1 ; N#Cc1ccc(-c2ccc(-c3nc4ccccc4c4c5ccccc5c5ccccc5c34)cc2)cc1]

Preparation of Compound 599

The title compound was prepared in the same manner as in the preparation method of Compound 37, except that 4-bromobenzonitrile was used instead of 2-bromo-4,6-diphenylpyrimidine (6.26 g, 66%).

### Preparation Example 48

### Preparation of Compound 600

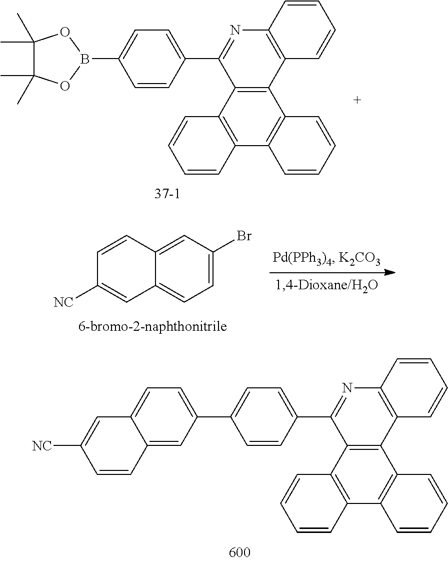 [SMILES: CC1(C)OB(c2ccc(-c3nc4ccccc4c4c5ccccc5c5ccccc5c34)cc2)OC1(C)C ; N#Cc1ccc2cc(Br)ccc2c1 ; N#Cc1ccc2cc(-c3ccc(-c4nc5ccccc5c5c6ccccc6c6ccccc6c45)cc3)ccc2c1]

The title compound was prepared in the same manner as in the preparation method of Compound 37, except that 6-bromo-2-naphthonitrile was used instead of 2-bromo-4,6-diphenylpyrimidine (5.68 g, 54%).

### Preparation Example 49

### Preparation of Compound 600

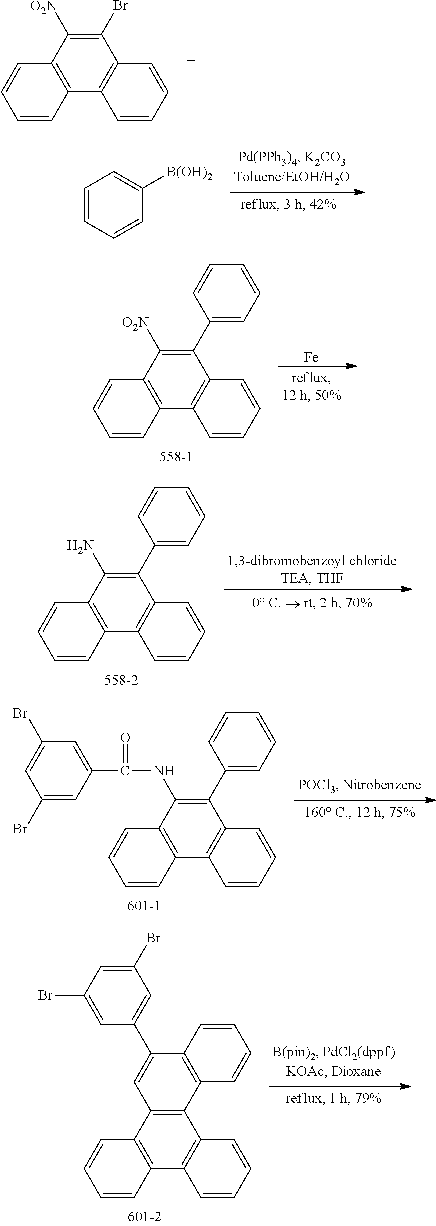

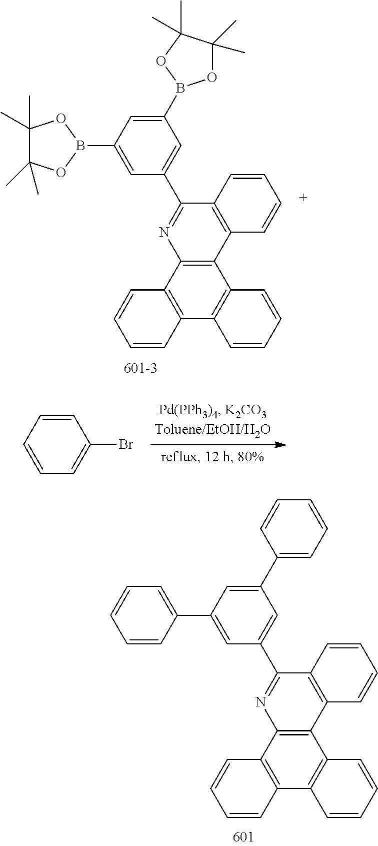 [SMILES: CC1(C)OB(c2cc(B3OC(C)(C)C(C)(C)O3)cc(-c3nc4c5ccccc5c5ccccc5c4c4ccccc34)c2)OC1(C)C ; Brc1ccccc1 ; c1ccc(-c2cc(-c3ccccc3)cc(-c3nc4c5ccccc5c5ccccc5c4c4ccccc34)c2)cc1]

Preparation of Compound 601-1

Compound 558-2 (60 g, 0.223 mol) was dissolved in THF (1,000 ml) in a one neck round bottom flask, and then trimethylamine (92.6 ml, 0.668 mol) was added dropwise thereto at room temperature. The reaction mixture was cooled to 0° C., and then 1,3-dibromobenzoyl chloride (73.1 g, 0.245 mol) was dissolved in 80 ml of THF, and the resulting solution was slowly added dropwise to the mixture at low temperature. The resulting mixture was then stirred at room temperature for 2 hours, and then H₂O was added thereto to terminate the reaction. The solvent was distilled under reduced pressure, and then the resulting product was extracted with methylene chloride (MC)/H₂O and dried over MgSO₄, and then resulting product was filtered and concentrated. The product was dissolved in a small amount of methylene chloride (MC), and then methanol was added to the solution to precipitate a solid, thereby obtaining an ivory solid Compound 601-1 (82.8 g, 70%).

Preparation of Compound 601-2

Compound 601-1 (82 g, 0.154 mol) was dissolved in nitrobenzene (1,600 ml) in a one neck round bottom flask, and then phosphorous oxychloride (POCl₃, 8.6 ml, 0.0924 mol) was added dropwise thereto at room temperature, and the resulting mixture was stirred under reflux for 12 hours. The mixed solution, in which the reaction had been terminated, was cooled to room temperature, was neutralized with a NaHCO₃ aqueous solution, and then the resulting product was extracted with methylene chloride (MC)/H₂O, dried over MgSO₄, and then filtered. The solvent was distilled under reduced pressure and concentrated, and then, a small amount of methylene chloride (MC) and an excessive amount of methanol were added to the solution to precipitate a solid; thereby obtaining a pale yellow solid Compound 601-2 (59.4 g, 75%).

Preparation of Compound 601-3

A 300 ml-mixed 1,4-dioxane solution of Compound 601-2 (15 g, 0.0292 mol), bis(pinacolato) diboron (22.2 g, 0.0876 mol), potassium acetate (KOAc, 14.3 g, 0.146 mol), and PdCl₂(dppf) (2.1 g, 0.00292 mol) was stirred under reflux under nitrogen for 3 hours in a two neck round bottom flask. The reaction mixture cooled to room temperature was extracted with methylene chloride (MC)/H₂O, dried over MgSO₄, and then filtered. After concentration, a small amount of EA slurry was post-filtered to obtain solid Compound 601-3 (14.1 g, 79%).

Preparation of Compound 601

A toluene/ethanol/H₂O (250 ml/150 ml/50 ml) mixed solution of Compound 601-3 (14.0 g, 0.023 mol), bromobenzene (10.8 g, 0.069 mol), K₂CO₃ (15.87 g, 0.115 mol), and Pd(PPh₃)₄ (2.6 g, 0.0023 mol) was stirred under reflux under nitrogen for 12 hours in a two neck round bottom flask. The reaction mixture cooled to room temperature was extracted with methylene chloride (MC)/H₂O, dried over MgSO₄, and then allowed to pass through silica gel. After concentration, a small amount of the resulting product was dissolved in EA, tetrahydrofuran (THF), and methanol, respectively, and then the resulting mixture was stirred for 3 hours or more and filtered to obtain white Compound 601 (9.36 g, 80%).

### Preparation Example 50

### Preparation of Compound 602

A toluene/ethanol/H₂O (250 ml/150 ml/50 ml) mixed solution of Compound 601-3 (14.0 g, 0.023 mol), 1-bromonaphthalene (14.2 g, 0.069 mol), K₂CO₃ (15.87 g, 0.115 mol), and Pd(PPh₃)₄ (2.6 g, 0.0023 mol) was stirred under reflux under nitrogen for 12 hours in a two neck round bottom flask. The reaction mixture cooled to room temperature was extracted with methylene chloride (MC)/H₂O, dried over MgSO₄, and then allowed to pass through silica gel. After concentration, a small amount of the resulting product was dissolved in EA, tetrahydrofuran (THF), and methanol, respectively, and then the resulting mixture was stirred for 3 hours or more and filtered to obtain white Compound 602 (9.09 g, 65%).

### Preparation Example 51

### Preparation of Compound 603

A toluene/ethanol/H₂O (250 ml/150 ml/50 ml) mixed solution of Compound 601-2 (11.8 g, 0.023 mol), dibenzo[b,d]furan-4-ylboronic acid (14.6 g, 0.069 mol), K₂CO₃ (15.87 g, 0.115 mol), and Pd(PPh₃)₄ (2.6 g, 0.0023 mol) was stirred under reflux under nitrogen for 12 hours in a two neck round bottom flask. The reaction mixture cooled to room temperature was extracted with methylene chloride (MC)/H₂O, dried over MgSO₄, and then allowed to pass through silica gel. After concentration, a small amount of the resulting product was dissolved in EA, tetrahydrofuran (THF), and methanol, respectively, and then the resulting mixture was stirred for 3 hours or more and filtered to obtain white Compound 603 (12.9 g, 82%).

### Preparation Example 52

### Preparation of Compound 604

A 250-ml toluene mixed solution of Compound 601-2 (11.8 g, 0.023 mol), 9H-carbazole (11.5 g, 0.069 mol), sodium tert-butoxide (11.1 g, 0.115 mol), Pd₂(dba)₃ (2.1 g, 0.0023 mol), and tri-tert-butyl phosphine (1.1 ml, 0.0046 mol) was stirred under reflux under nitrogen for 12 hours in a two neck round bottom flask. The reaction mixture cooled to room temperature was extracted with methylene chloride (MC)/H₂O, dried over MgSO₄, and then allowed to pass through silica gel. After concentration, a small amount of the resulting product was dissolved in EA, tetrahydrofuran (THF), and methanol, respectively, and then the resulting mixture was stirred for 3 hours or more and filtered to obtain white Compound 604 (12.9 g, 82%).

### Preparation Example 53

### Preparation of Compound 605

A toluene/ethanol/H₂O (250 ml/150 ml/50 ml) mixed solution of Compound 601-3 (14.0 g, 0.023 mol), 2-bromodibenzo[b,d]thiophene (18.2 g, 0.069 mol), K₂CO₃ (15.87 g, 0.115 mol), and Pd(PPh₃)₄ (2.6 g, 0.0023 mol) was stirred under reflux under nitrogen for 12 hours in a two neck round bottom flask. The reaction mixture cooled to room temperature was extracted with methylene chloride (MC)/H₂O, dried over MgSO₄, and then allowed to pass through silica gel. After concentration, a small amount of the resulting product was dissolved in EA, tetrahydrofuran (THF), and methanol, respectively, and then the resulting mixture was stirred for 3 hours or more and filtered to obtain white Compound 605 (11.6 g, 70%).

### Preparation Example 54

### Preparation of Compound 606

A toluene/ethanol/H₂O (250 ml/150 ml/50 ml) mixed solution of Compound 601-3 (14.0 g, 0.023 mol), 2-bromo-9,10-phenanthroline (17.9 g, 0.069 mol), K₂CO₃ (15.87 g, 0.115 mol), and Pd(PPh₃)₄ (2.6 g, 0.0023 mol) was stirred under reflux under nitrogen for 12 hours in a two neck round bottom flask. The reaction mixture cooled to room temperature was extracted with methylene chloride (MC)/H₂O, dried over MgSO₄, and then allowed to pass through silica gel. After concentration, a small amount of the resulting product was dissolved in EA, tetrahydrofuran (THF), and methanol, respectively, and then the resulting mixture was stirred for 3 hours or more and filtered to obtain white Compound 606 (7.7 g, 47%).

### Preparation Example 55

### Preparation of Compound 607

A toluene/ethanol/H₂O (250 ml/150 ml/50 ml) mixed solution of Compound 601-3 (14.0 g, 0.023 mol), 1-(4-bromophenyl)-2-phenyl-1H-benzo[d]imidazole (24.1 g, 0.069 mol), K₂CO₃ (15.87 g, 0.115 mol), and Pd(PPh₃)₄ (2.6 g, 0.0023 mol) was stirred under reflux under nitrogen for 12 hours in a two neck round bottom flask. The reaction mixture cooled to room temperature was extracted with methylene chloride (MC)/H₂O, dried over MgSO₄, and then allowed to pass through silica gel. After concentration, a small amount of the resulting product was dissolved in EA, tetrahydrofuran (THF), and methanol, respectively, and then the resulting mixture was stirred for 3 hours or more and filtered to obtain white Compound 607 (20.5 g, 66%).

### Preparation Example 56

### Preparation of Compound 608

A toluene/ethanol/H₂O (250 ml/150 ml/50 ml) mixed solution of Compound 601-2 (11.8 g, 0.023 mol), phenanthren-9-yl boronic acid (15.3 g, 0.069 mol), K₂CO₃ (15.87 g, 0.115 mol), and Pd(PPh₃)₄ (2.6 g, 0.0023 mol) was stirred under reflux under nitrogen for 12 hours in a two neck round bottom flask. The reaction mixture cooled to room temperature was extracted with methylene chloride (MC)/H₂O, dried over MgSO₄, and then allowed to pass through silica gel. After concentration, a small amount of the resulting product was dissolved in EA, tetrahydrofuran (THF), and methanol, respectively, and then the resulting mixture was stirred for 3 hours or more and filtered to obtain white Compound 603 (11.6 g, 71%).

### Preparation Example 57

### Preparation of Compound 609

A toluene/ethanol/H₂O (250 ml/150 ml/50 ml) mixed solution of Compound 601-3 (14.0 g, 0.023 mol), 4-bromo-9,9′-spirobi[fluorene] (27.3 g, 0.069 mol), K₂CO₃ (15.87 g, 0.115 mol), and Pd(PPh₃)₄ (2.6 g, 0.0023 mol) was stirred under reflux under nitrogen for 12 hours in a two neck round bottom flask. The reaction mixture cooled to room temperature was extracted with methylene chloride (MC)/H₂O, dried over MgSO₄, and then allowed to pass through silica gel. After concentration, a small amount of the resulting product was dissolved in EA, tetrahydrofuran (THF), and methanol, respectively, and then the resulting mixture was stirred for 3 hours or more and filtered to obtain white Compound 607 (12.4 g, 55%).

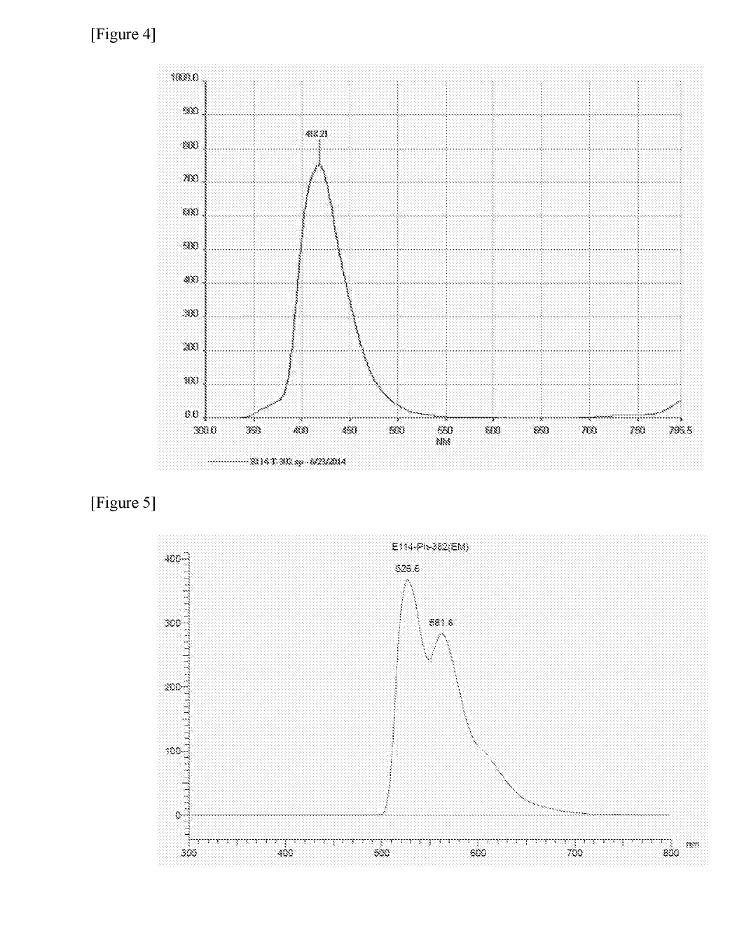

The compounds were prepared according to the above-described preparation examples. Synthesis thereof was checked, and the check results were as listed in Table 2 and illustrated in FIG. 4 to FIG. 25.

The following Table 2 lists ¹H NMR (CDCl₃, 200 Mz) measurement data and measurement data obtained by an FD-spectrometer (FD-MS: Field desorption mass spectrometry).

TABLE 2

| | | MS/FAB | |
|---|---|---|---|
| Compound | ¹H NMR(CDCl₃, 200 Mz) | found | calculated |
| 2 | 9.08(2H,d), 8.17(1H,s), 8.37(1H,d), 8.19(3H,m), | 405.50 | 405.15 |
| | 8.06(2H,m), 8.00(1H,m), 7.94(1H,d), 7.85(1H,t) | | |
| 4 | 9.08(2H,d), 8.95(1H,d), 8.69(2H,d), 8.50(1H,d), | 481.60 | 481.18 |
| | 8.19(4H,m), 8.09(1H,d), 7.94(1H,d), 7.85(1H,t), 7.77- | | |
| | 7.62(6H,m), 7.52(1H,t), 7.39(1H,t), 7.25(2H,d) | | |
| 6 | 9.08(2H,d), 8.69(4H,d), 8.37(1H,d), 8.19(3H,m), | 508.62 | 508.19 |
| | 7.94(1H,d), 7.85(5H,m), 7.70-7.62(5H,m), 7.38(1H,t), | | |
| | 7.14(1H,d), 6.90(1H,t) | | |
| 7 | 9.08(2H,d), 8.69(2H,d), 8.35(3H,m), 8.19(3H,m), | 508.62 | 508.19 |
| | 7.94(1H,d), 7.85(3H,m), 7.73-7.61(7H,m), 7.38(1H,t), | | |
| | 7.14(1H,d), 6.90(1H,t) | | |
| 8 | 9.08(2H,d), 8.69(2H,d), 8.19(3H,m), 8.04(3H,s), | 583.73 | 583.13 |
| | 7.94(1H,d), 7.85(3H,m), 7.73(7H,m), 7.62(2H,m), | | |
| | 7.49(4H,m), 7.41(2H,m) | | |
| 9 | 9.08(2H,d), 8.70(6H,m), 8.19(3H,m), 8.04(3H, s), | 585.71 | 585.22 |
| | 7.98(5H,m), 7.85(3H,m), 7.70(3H,t), 7.62(2H,d) | | |
| 12 | 9.08(2H,d), 8.99(1H,s), 8.69(2H,d), 8.42(1H,d), | 783.97 | 783.29 |
| | 8.19(5H,m), 8.08(4H,m), 7.97(3H,m), 7.85(3H,m), 7.70- | | |
| | 7.55(11H,m), 7.47-7.32 (5H,m) | | |
| 16 | 9.08(2H,d), 8.69(2H,d), 8.56(1H,d,), 8.19(3H,m), | 623.76 | 623.24 |
| | 7.95(3H,m), 7.84(4H,m), 7.70(3H,m), 7.62(3H,m), | | |
| | 7.50(3H,m), 7.38(2H,m), 7.26(3H,m) | | |
| 18 | 9.08(2H,d), 8.36(2H,d), 8.19(3H,m), 7.95(3H,m), | 555.62 | 555.18 |
| | 7.85(1H,t), 7.77(4H,m), 7.65(5H,m), 7.51(6H,m) | | |
| 22 | 9.27(2H,d), 8.89(1H,d), 8.76(2H,t), 8.62(1H,d), 8.39(1H,d), | 482.59 | 482.18 |
| | 8.32(1H,d), 8.16(1H,d), 7.92(4H,m), 7.82(3H,m), | | |
| | 7.76(3H,m), 7.60(3H,m), 7.29(1H,t) | | |
| 32 | 9.08(2H,d), 8.70(10H,m), 8.19(5H,m), 8.00(4H,d), | 738.89 | 738.28 |
| | 7.94(1H,d), 7.85(7H,m), 7.70(3H,m), 7.62(2H,t) | | |
| 35 | 9.08(2H,d), 8.70(10H,m), 8.19(5H,m), 8.00(4H,d), | 738.89 | 738.28 |
| | 7.94(1H,d), 7.85(3H,m), 7.70(3H,t), 7.62(2H,t), 7.25(4H,m) | | |
| 37 | 9.08(2H,d), 8.69(2H,d), 8.20(4H,m), 7.95(7H,m), | 585.71 | 585.22 |
| | 7.85(1H,t), 7.70(3H,m), 7.62-7.49(8H,m) | | |
| 38 | 9.08(2H,d), 8.69(2H,d), 8.30(2H,d), 8.20(4H,m), | 661.81 | 661.25 |
| | 7.95(5H,m), 7.85(3H,m), 7.70-7.41(13H, m) | | |
| 41 | 9.08(2H,d), 8.69(2H,d), 8.33(4H,m), 8.20(4H,m), | 585.71 | 585.22 |
| | 7.94(3H,m), 7.85(1H,t), 7.70-7.49(11H,m) | | |
| 43 | 9.08(2H,d), 8.69(2H,d), 8.30(2H,d), 8.17(4H,m), | 635.77 | 635.24 |
| | 7.95(3H,m), 7.85(5H,m), 7.75-7.70(5H,m), 7.62- | | |
| | 7.58(3H,m), 7.49(2H,m), 7.41(1H,m) | | |
| 44 | 9.08(2H,d), 8.69(2H,d), 8.36(4H,m), 8.19(3H,m), | 586.70 | 586.22 |
| | 7.95(3H,m), 7.85(1H,t), 7.70(3H,t), 7.62(2H,t), 7.50(6H,m) | | |
| 46 | 9.08(2H,d), 8.69(2H,d), 8.19(3H,m), 7.95(7H,m), | 738.89 | 738.28 |
| | 7.85(1H,t), 7.75-7.62(9H,m), 7.49(4H,t), 7.41(2H,m), | | |
| | 7.25(4H,d) | | |
| 54 | 9.08(2H,d), 8.69(2H,d), 8.20-8.13(4H,m), 8.06(2H,m), | 674.81 | 674.25 |
| | 7.95(3H,m), 7.84(5H,m), 7.70(3H,t), 7.62-7.49(6H,m) | | |
| 55 | 9.08(2H,d), 8.69(4H,d), 8.19(6H,m), 7.94(2H,d), | 708.86 | 708.26 |
| | 7.85(6H,m), 7.70(6H,m), 7.62(4H,m) | | |
| 57 | 9.08(2H,d), 8.33(2H,m), 8.19(3H,m), 8.08(2H,m), | 481.60 | 481.18 |
| | 7.97(2H,m), 7.85(1H,t), 7.73-7.55(10H,m), 7.38(1H,d) | | |
| 59 | 9.08(2H,d), 8.33(2H,m), 8.19(3H,m), 7.94(2H,m), | 507.64 | 507.20 |
| | 7.85(1H,t), 7.75-7.61(12H,m), 7.49(2H,t), 7.41(1H,m) | | |
| 60 | 9.08(2H,d), 8.71(2H,d), 8.33(2H,m), 8.19(2H,d), | 508.62 | 508.19 |
| | 8.00(2H,d), 7.94(2H,m), 7.85(1H,t), 7.73-7.61(10H,m) | | |
| 61 | 9.08(2H,d), 8.69(2H,d), 8.35(3H,m), 8.49(3H,m), | 508.62 | 508.19 |
| | 7.94(1H,d), 7.85(3H,m), 7.71(4H,m), 7.62(3H,m), | | |
| | 7.38(1H,t), 7.14(1H,d), 6.90(1H,t) | | |
| 63 | 9.08(2H,d), 8.33(2H,m), 8.19(3H,m), 8.04(3H,s), | 583.73 | 583.23 |
| | 7.94(1H,d), 7.85(1H,t), 7.73(8H,m), 7.62(3H,m), | | |
| | 7.49(4H,m), 7.41(2H,m) | | |
| 67 | 9.08(2H,d), 8.99(1H,s), 8.42(1H,d), 8.33(2H,m), | 783.97 | 738.29 |
| | 8.19(SH,m), 8.08(4H,m), 7.97(3H,m), 7.85(1H,t), 7.73- | | |
| | 7.55(13H,m), 7.45(3H,m), 7.38(2H,m) | | |
| 70 | 9.08(2H,d), 8.33(2H,m), 8.19(3H,m), 8.09(1H,d), 7.94- | 669.83 | 669.25 |
| | 7.55(15H,m), 7.45(2H,m), 7.38(1H,d),7.28(5H,m) | | |
| 71 | 9.08(2H,d), 8.56(1H,d), 8.33(2H,m), 8.19(3H,m), | 623.76 | 623.24 |
| | 7.95(3H,m), 7.83(2H,m),7.72(4H,m), 7.62(4H,m), | | |
| | 7.50(3H,m), 7.38(2H,m), 7.27(3H,m) | | |
| 73 | 9.08(2H,d), 8.50(2H,m), 8.19(3H,m), 7.94(1H,d), | 555.62 | 555.18 |
| | 7.86(2H,m), 7.74(8H,m), 7.62(2H,t), 7.51(6H,m) | | |
| 77 | 9.08(2H,d), 8.33(2H,m), 8.26(1H,s), 8.19(3H,m), | 482.59 | 482.18 |
| | 8.08(2H,m), 7.94(1H,d), 7.85(1H,t), 7.73-7.61(9H,m) | | |
| 87 | 9.08(2H,d), 8.70(8H,m), 8.33(2H,m), 8.20(5H,m), | 738.89 | 738.28 |
| | 8.00(4H,d), 7.44(1H,d), 7.85(5H,m), 7.71(4H,m), | | |
| | 7.62(3H,m) | | |
| 90 | 9.08(2H,d), 8.71(7H,m), 8.33(2H,m), 8.19(5H,m), | 738.89 | 738.28 |
| | 8.00(4H,d), 7.94(1H,d), 7.85(3H,m), 7.71(4H,m), | | |
| | 7.62(2H,t), 7.25(4H,d) | | |
| 92 | 9.08(2H,d), 8.35(3H,m), 8.20(4H,m), 7.94(5H,m), | 585.71 | 585.22 |
| | 7.85(1H,t), 7.73-7.49(12H,m) | | |
| 93 | 9.08(2H,d), 8.33(5H,m), 8.20(4H,m), 7.94(3H,m), | 661.81 | 661.25 |
| | 7.85(3H,m), 7.73(6H,m), 7.62-7.41(8H,m) | | |
| 96 | 9.08(2H,d), 8.34(4H,m), 8.20(4H,m), 7.94(4H,m), | 585.71 | 585.22 |
| | 7.85(1H,t), 7.73-7.49(12H,m) | | |
| 98 | 9.08(2H,d), 8.35(5H,m), 8.17(4H,m), 7.94(1H,d), | 635.77 | 635.24 |
| | 7.85(5H,t), 7.73-7.58(7H,m), 7.49(2H,m), 7.41(1H,m) | | |
| 99 | 9.08(2H,d), 8.35(7H,m), 8.19(3H,m), | 586.70 | 586.22 |
| | 7.94(1H,d),7.85(1H,t), 7.71(4H,m), 7.62(2H,t), 7.50(6H,m) | | |
| 101 | 9.08(2H,d), 8.58(2H,m), 8.35(3H,m), 8.20(5H,m), | 738.89 | 738.28 |
| | 7.94(1H,d), 7.85(3H,m), 7.70(6H,m), 7.40(2H,t) | | |
| 109 | 9.08(2H,d), 8.33(2H,m), 8.15(4H,m), 8.06(2H,m), | 674.81 | 674.25 |
| | 7.95(3H,m), 7.84(2H,m), 7.73-7.54(11H,m), 7.25(2H,d), | | |
| | 6.53(1H,s) | | |
| 110 | 9.08(4H,d), 8.69(2H,d), 8.33(2H,m), 8.19(6H,m), | 708.86 | 708.26 |
| | 7.94(2H,d), 7.85(4H,m), 7.73-7.61(12H,m) | | |
| 111 | 9.08(2H,d), 9.01(1H,s), 8.60(1H,m), 8.19(5H,m), | 636.80 | 636.26 |
| | 7.94(1H,d), 7.85(1H,t), 7.74-7.50(12H,m), 7,38(1H,t), | | |
| | 7.20(1H,t), 1.69(12H,s) | | |
| 112 | 9.08(2H,d), 9.01(1H,s), 8.60(1H,m), 8.20(6H,m), | 760.94 | 760.29 |
| | 7.49(1H,d), 7.85(1H,t), 7.74-7.50(12H,m), 7.38(1H,t), 7.26- | | |
| | 7.18(7H,m), 7.10(4H,m) | | |
| 113 | 9.08(2H,d), 8.60(1H,m), 8.45(1H,d), 8.19(4H,m), | 626.78 | 626.18 |
| | 7.94(2H,m), 7.86(2H,m), 7.78(1H,s), 7.70-7.49(11H,m), | | |
| | 7.20(1H,t) | | |
| 115 | 9.08(2H,d), 8.60(1H,m), 8.55(1H,d), 8.19(5H,m), | 685.83 | 685.25 |
| | 7.94(2H,d), 7.85(1H,t), 7.70-7.50(15H,m), 7.40(1H,s), | | |
| | 7.35(1H,t), 7.18(2H,m) | | |
| 130 | 9.08(2H,d), 8.60(1H,m), 8.55(1H,d), 8.19(4H,m), | 685.83 | 685.25 |
| | 8.12(114,d), 7.94(3H,d), 7.85(1H,t), 7.70-7.50(13H,m), | | |
| | 7.35(2H,t), 7.16(2H,t) | | |
| 131 | 9.08(2H,d), 8.60(1H,m), 8.55(1H,d), 8.20(6H,m), | 636.80 | 636.26 |
| | 7.90(4H,m), 7.70-7.54(9H,m), 7.36(2H,m), 7.16(1H,t), | | |
| | 1.69(12H,s) | | |
| 132 | 9.08(2H,d), 8.60(1H,m), 8.55(1H,d), 8.45(1H,d), | 626.78 | 626.18 |
| | 8.19(4H,m), 8.05(1H,d), 7.94(3H,m), 7.85(1H,t), 7.70- | | |
| | 7.49(10H,m), 7.35(1H,t), 7.16(1H,t) | | |
| 134 | 9.08(2H,d), 8.60(1H,m), 8.55(1H,d), 8.19(5H,m), | 685.83 | 685.25 |
| | 7.94(2H,m), 7.85(1H,t), 7.70-7.50(15H,m), 7.40(1H,s), | | |
| | 7.35(1H,t), 7.18(2H,m) | | |
| 135 | 9.08(2H,d), 8.60(1H,m), 8.45(1H,d), 8.19(5H,m), | 626.78 | 626.18 |
| | 7.94(2H,m), 7.86(2H,m), 7.78(1H,s), 7.70-7.50(11H,m), | | |
| | 7.20(1H,t) | | |
| 137 | 9.08(2H,d), 8.60(1H,m), 8.19(7H,m), 7.94(1H,d), | 760.94 | 760.29 |
| | 7.87(2H,m), 7.74-7.50(11H,m), 7.38(1H,t), 7.26- | | |
| | 7.10(11H,m) | | |
| 146 | 9.08(2H,d), 8.55(1H,d), 8.30(1H,d), 8.24(2H,m), | 636.80 | 636.26 |
| | 8.19(3H,m), 7.93(4H,m), 7.85(1H,t), 7.72(SH,m), | | |
| | 7.60(3H,m), 7.36(2H,m), 7.16(1H,t), 1.69(12H,s) | | |
| 147 | 9.08(2H,d), 8.55(1H,d), 8.30(2H,d), 8.24(2H,m), | 760.94 | 760.29 |
| | 8.19(3H,m), 7.93(4H,m), 7.85(1H,t), 7.74-7.57(8H,m), | | |
| | 7.37(2H,m), 7.26-7.10(11H,m) | | |
| 148 | 9.08(2H,d), 8.55(1H,d), 8.45(1H,d), 8.30(2H,d), | 626.78 | 626.18 |
| | 8.19(3H,m), 7.93(5H,m), 7.85(2H,m), 7.78(1H,s), 7.70- | | |
| | 7.49(7H,m), 7.35(1H,t), 7.16(1H,t) | | |
| 150 | 9.08(2H,d), 8.55(1H,d), 8.30(2H,d), 8.19(4H,m), | 685.83 | 685.25 |
| | 7.93(4H,m), 7.85(1H,t), 7.70-7.50(13H,m), 7.40(1H,s), | | |
| | 7.35(1H,t), 7.18(2H,m) | | |
| 165 | 9.08(2H,d), 8.55(2H,d), 8.30(2H,d), 8.19(3H,m), | 685.83 | 685.25 |
| | 8.12(1H,d), 7.93(5H,m), 7.85(1H,t), 7.70-7.57(8H,m), | | |
| | 7.50(2H,d), 7.35(2H,t), 7.16(2H,t) | | |
| 166 | 9.08(2H,d), 8.55(1H,d), 8.30(2H,d), 8.24-8.17(5H,m), 7.93 | 636.80 | 636.26 |
| | (5H,m), 7.85(1H,t), 7.74-7.57(7H,m), 7.37(2H,m), | | |
| | 7.16(1H,t), 1.69 (12H,s) | | |
| 167 | 9.08(2H,d), 8.55(1H,d), 8.45(1H,d), 8.30(2H,d), | 626.78 | 626.18 |
| | 8.19(3H,m), 8.05(1H,d), 7,93(5H,m), 7.85(1H,t), 7.70- | | |
| | 7.49(8H,m), 7.35(1H,t), 7.16(1H,t) | | |
| 169 | 9.08(2H,d), 8.55(1H,d), 8.30(2H,d), 8.19(4H,m), | 685.83 | 685.25 |
| | 7.93(4H,m), 7.85(1H,t), 7.70-7.50(13H,m), 7.40(1H,s), | | |
| | 7.35(1H,t), 7.18(2H,m) | | |
| 170 | 9.08(2H,d), 8.55(1H,d), 8.45(1H,d), 8.30(2H,d), | 626.78 | 626.18 |
| | 8.19(3H,m), 7.93(5H,m), 7.86(2H,m), 7.78(1H,s), 7.70- | | |
| | 7.49(7H,m), 7.35(1H,t), 7.16(1H,t) | | |
| 172 | 9.08(2H,d), 8.55(1H,d), 8.30(2H,d), 8.20(4H,m), 7.94- | 760.94 | 760.29 |
| | 7.85(6H,m), 7.74-7.57(7H,m), 7.49(1H,s), 7.36(2H,m), | | |
| | 7.26-7.16(11H,m) | | |
| 181 | 9.08(2H,d), 8.84(1H,d), 8.69(2H,d), 8.27(1H,d), | 531.66 | 531.20 |
| | 8.19(3H,m), 8.05(1H,s), 7.94-7.85(3H,m), 7.70- | | |
| | 7.62(9H,m), 7.25(2H,d) | | |
| 184 | 9.27(1H,s), 9.08(2H,d), 8.79(1H,d), 8.69(2H,d), 8.69(2H,d), | 581.72 | 581.21 |
| | 8.35(4H,m), 8.19(3H,m), 7.94(1H,d),7.85(3H,m), 7.70- | | |
| | 7.62(9H,m), 7.52(1H,d) | | |
| 185 | 9.08(3H,m), 8.84(1H,d), 8.33(2H,m), 8.27(1H,d), | 531.66 | 531.20 |
| | 8.19(3H,m), 8.05(1H,s), 7.94-7.85(3H,m), 7.73- | | |
| | 7.62(11H,m) | | |
| 188 | 9.60(1H,d), 9.27(1H,s), 9.08(2H,d), 8.35(5H,m), | 581.72 | 581.21 |
| | 8.19(3H,m), 7.94(1H,d), 7.85(1H,t), 7.73-7.61(11H,m), | | |
| | 7.51(2H,d) | | |
| 189 | 9.08(2H,d), 8.55(1H,d), 8.30(2H,d), 8.19(4H,m), | 520.63 | 520.19 |
| | 7.93(4H,m), 7.85(1H,t), 7.60(3H,m), 7.50(1H,t), 7.35(1H,t), | | |
| | 7.18(2H,m) | | |
| 199 | 9.08(2H,d), 8.60(1H,s), 8.55(1H,d), 8.19(5H,m), | 520.63 | 520.19 |
| | 7.94(2H,d), 7.85(1H,t), 7.70-7.58(8H,m), 7.50(1H,t), | | |
| | 7.35(1H,t), 7.18(2H,m) | | |
| 209 | 9.08(2H,d), 8.69(2H,d), 8.55(2H,d), 8.19(7H,m), | 761.93 | 761.28 |
| | 7.94(3H,d), 7.85(3H,m), 7.70(3H,m), 7.60(SH,m), | | |
| | 7.50(2H,t), 7.35(2H,t), 7.18(4H,m) | | |
| 210 | 9.08(2H,d), 8.55(1H,d), 8.30(3H,d), 8.20-8.13(5H,m), 7.99- | 761.93 | 761.28 |
| | 7.85(8H,m), 7.77-7.58(10H,m), 7.50(3H,m), 7.35(1H,t), | | |
| | 7.18(2H,m) | | |
| 220 | 9.08(2H,d), 8.70(3H,m), 8.39(1H,d), 8.23(1H,s), | 586.70 | 586.20 |
| | 8.17(2H,d), 7.95(6H,m), 7.70(2H,t), 7.62-7.41(9H,m) | | |
| 224 | 9.45(1H,s), 9.08(2H,d), 8.82(1H,d), 8.69(2H,d), 8.23(1H,s), | 586.70 | 586.20 |
| | 8.17(2H,d), 7.95(6H,m), 7.70-7.49(11H,m) | | |
| 228 | 9.08(2H,d), 8.69(2H,d), 8.51(1H,d), 8.23(1H,s), 8.20(1H,d), | 635.77 | 635.24 |
| | 8.17(2H,d), 8.11(1H,d), 7.95(6H,m), 7.90(1H,d), 7.72- | | |
| | 7.49(12H,m) | | |
| 232 | 9.08(2H,d), 8.69(2H,d), 8.54(1H,d), 8.23(1H,s), 8.17(2H,d), | 635.77 | 635.24 |
| | 8.08(2H,m), 7.99-7.94(7H,m), 7.70-7.49(12H,m) | | |
| 236 | 9.16(1H,s), 9.09(2H,d), 8.69(2H,d), 8.23(1H,s), 8.20(1H,s), | 635.77 | 635.24 |
| | 8.15(3H,m), 7.95(7H,m), 7.85(1H,t), 7.70(2H,t), 7.62(1H,t), | | |
| | 7.55-7.49(8H,m) | | |
| 240 | 9.16(1H,s), 9.09(2H,d), 8.69(2H,d), 8.23(1H,s), 8.20(1H,d), | 635.77 | 635.24 |
| | 8.15(3H,m), 7.95(7H,m), 7.85(1H,t), 7.70(2H,t), 7.62(1H,t), | | |
| | 7.55-7.49(10H,m) | | |
| 244 | 8.94(1H,d), 8.69(2H,d), 8.54-8.46(3H,m), 8.23(1H,s), | 586.70 | 586.20 |
| | 8.20(1H,d), 7.95(7H,m), 7.85(1H,t), 7.72-7.49(10H,m) | | |
| 248 | 9.08(1H,d), 9.00(2H,m), 8.69(2H,d), 8.23(1H,s), | 586.70 | 586.20 |
| | 8.17(2H,d), 8.11(1H,d), 7.95(6H,m), 7.70-7.49(11H,m) | | |
| 252 | 9.39(1H,s), 9.08(1H,d), 8.98(1H,d), 8.75(1H4), 8.69(2H,d), | 586.70 | 586.20 |
| | 8.23(1H,s), 8.17(1H,d), 8.11(1H,d), 7.95(6H,m), | | |
| | 7.78(1H,d), 7.70-7.49(10H,m) | | |
| 256 | 9.08(1H,d), 8.98(1H,d), 8.69(2H,d), 8.54(1H,d), 8.23(1H,s), | 635.77 | 635.24 |
| | 8.17(1H,d), 8.11(1H,d), 7.99-7.94(7H,m), 7.83(1H,d), 7.70- | | |
| | 7.49(13H,m) | | |
| 260 | 9.08(1H,d), 8.98(1H,d), 8.69(2H,d), 8.54(1H,d), 8.23(1H,s), | 635.77 | 635.24 |
| | 8.17(1H,d), 8.11(1H,d), 7.99-7.94(8H,m), 7.70- | | |
| | 7.49(13H,m) | | |
| 264 | 9.16(1H,s), 9.10(1H,s), 8.98(1H,d), 8.69(2H,d), 8.23(1H,s), | 635.77 | 635.24 |
| | 8.12(3H,m), 7.99-7.94(7H,m), 7.83(1H,d), 7.68- | | |
| | 7.49(12H,m) | | |
| 268 | 8.98(2H,d), 8.69(2H,d), 8.23(1H,s), 8.17(1H,d), 7.99- | 635.77 | 635.24 |
| | 7.90(9H,m), 7.83(1H,d), 7.70-7.49(13H,m) | | |
| 272 | 9.08(1H,d), 8.98(1H,d), 8.69(2H,d), 8.23(1H,s), 8.17(1H,d), | 635.77 | 635.24 |
| | 7.99-7.90(9H,m), 7.83(1H,d), 7.70-7.49(13H,m) | | |
| 324 | 9.08(2H,d), 8.69(2H,d), 8.33(6H,m), 8.20(4H,m), | 661.81 | 661.25 |
| | 7.94(1H,d), 7.85(3H,m), 7.75-7.62 (7H,m), 7.50(5H,m), | | |
| | 7.41(1H,t) | | |
| 337 | 9.08(2H,d), 8.69(2H,d), 8.33(6H,m), 8.20(4H,m), | 737.91 | 737.28 |
| | 7.94(1H,d), 7.85(7H,m), 7.75-7.62 (7H,m), 7.50(5H,m), | | |
| | 7.41(1H,t) | | |
| 376 | 9.08(2H,d), 8.69(2H,d), 8.33(4H,m), 8.15(4H,m), | 559.67 | 559.20 |
| | 7.94(1H,d), 7.85(3H,m), 7.70(3H,m), 7.60(3H,m), | | |
| | 7.50(3H,m) | | |
| 385 | 9.08(2H,d), 8.80(1H,d), 8.70(5H,d), 8.45(1H,d), 8.19(4H,t), | 533.63 | 533.19 |
| | 7.94(1H,d), 7.90(1H,d), 7.85(1H,t), 7.70(3H,m), | | |
| | 7.60(3H,m), 7.29(1H,d) | | |
| 388 | 9.08(2H,d), 8.69(2H,d), 8.48(1H,d), 8.28(3H,m), | 547.66 | 547.20 |
| | 8.19(3H,m), 7.94(1H,d), 7.85(4H,m), 7.70(3H,t), | | |
| | 7.62(2H,t), 7.54(1H,d), 7.21(1H,t), 7.16(1H,t) | | |
| 396 | 9.08(2H,d), 8.69(2H,d), 8.56(1H,d), 8.19(3H,m), | 575.71 | 575.24 |
| | 7.94(1H,d), 7.85-7.62(12H,m), 7.54(1H,d), 7.25(2H,m), | | |
| | 2.85(2H,q), 1.30(3H,t) | | |
| 421 | 9.08(2H,d), 8.33(2H,m), 8.19(4H,m), 8.19(4H,m), 8.02- | 564.71 | 564.17 |
| | 7.94(4H,m), 7.85(1H,t), 7.72(4H,m), 7.62(3H,m), | | |
| | 7.52(2H,m), 7.25(2H,d) | | |
| 422 | 9.08(2H,d), 8.55(2H,d), 8.33(2H,m), 8.18(7H,m), | 761.93 | 761.28 |
| | 7.94(3H,d), 7.85(1H,t), 7.72(4H,m), 7.60(6H,m), | | |
| | 7.50(2H,m), 7.35(2H,m), 7.18(4H,m) | | |
| 465 | 9.08(2H,d), 8.33(6H,m), 8.19(4H,m), 7.94(2H,d), | 661.81 | 661.25 |
| | 7.85(3H,m), 7.75-7.62(8H,m), 7.50(5H,m), 7.41(1H,m) | | |
| 477 | 9.08(2H,d), 8.33(8H,m), 8.20(4H,m), 7.94(1H,d), | 737.91 | 737.28 |
| | 7.85(5H,m), 7.73(6H,m), 7.62(3H,m), 7.50(5H,m), | | |
| | 7.41(1H,t) | | |
| 515 | 9.08(2H,d), 8.35(4H,m), 8.15(4H,m), 7.94(1H,d), | 559.67 | 559.20 |
| | 7.85(4H,m), 7.73-7.58(7H,m), 7.50(3H,m) | | |
| 528 | 9.08(2H,d), 8.80(1H,d), 8.70(3H,m), 8.45(1H,d), | 609.73 | 609.22 |
| | 8.33(2H,m), 8.19(4H,m), 7.94(1H,d), 7.85(4H,m), 7.73- | | |
| | 7.56(8H,m), 7.29(1H,d) | | |
| 530 | 9.08(2H,d), 8.48(1H,d), 8.30(5H,m), 8.19(3H,m), | 547.66 | 547.20 |
| | 7.94(1H,d), 7.85(3H,m), 7.71(4H,m), 7.61(3H,m), | | |
| | 7.54(1H,d), 7.21(1H,t), 6.86(1H,t) | | |
| 538 | 9.08(2H,d), 8.56(1H,d), 8.33(2H,m), 8.19(3H,m), | 575.71 | 575.24 |
| | 7.94(1H,m), 7.85-7.70(9H,m), 7.61(3H,m), 7.54(1H,d), | | |
| | 7.28(1H,t), 7.21(1H,t), 2.85(2H,q), 1.30(3H,t) | | |
| 557 | 9.08(1H,d), 8.98(1H,d), 8.69(2H,d), 8.33(4H,m), | 585.71 | 585.22 |
| | 8.23(1H,s), 8.17(1H,d), 8.11(1H,d), 7.96(3H,m), | | |
| | 7.83(1H,d), 7.70-7.49(12H,m) | | |
| 558 | 9.08(1H,d), 8.98(1H,d), 8.69(2H,d), 8.33(4H,m), | 585.71 | 585.22 |
| | 8.23(1H,s), 8.17(114,d), 8.11(1H,d), 7.96(3H,m), | | |
| | 7.83(1H,d), 7.70-7.49(12H,m) | | |
| 559 | 9.08(1H,d), 8.98(1H,d), 8.69(2H,d), 8.33(4H,m), | 661.81 | 661.25 |
| | 8.23(1H,s), 8.17(1H,d), 8.11(1H,d), 7.96(3H,m), | | |
| | 7.84(5H,m), 7.70-7.49(12H,m) | | |
| 560 | 9.08(1H,d), 8.98(1H,d), 8.69(2H,d), 8.33(6H,m), | 661.82 | 661.25 |
| | 8.23(1H,s), 8.17(1H,d), 8.11(1H,d), 7.99(1H,d), | | |
| | 7.85(2H,m), 7.75-7.61(9H,m), 7.50(5H,m), 7.41(1H,t) | | |
| 561 | 9.08(1H,d), 8.98(1H,d), 8.69(2H,d), 8.33(4H,m), | 737.91 | 737.28 |
| | 8.23(1H,s), 8.17(1H,d), 8.11(1H,d), 7.99(1H,d), | | |
| | 7.85(7H,m), 7.75-7.61(12H,m), 7.50(5H,m), 7.41(1H,t) | | |
| 562 | 9.19(1H,s) 9.08(1H,d), 8.98(1H,d), 8.74(1H,d), 8.69(2H,d), | 586.70 | 586.22 |
| | 8.35(5H,m), 8.17(1H,d), 8.11(1H,d), 8.00(2H,m), | | |
| | 7.83(1H,d), 7.70-7.59(7H,m) | | |
| 563 | 9.08(1H,d), 8.98(1H,d), 8.69(2H,d), 8.36(4H,m), | 586.70 | 586.22 |
| | 8.17(1H,d), 8.11(1H,d), 7.96(3H,m), 7.83(1H,d), 7.70- | | |
| | 7.61(6H,m), 7.50(6H,m) | | |
| 564 | 9.08(1H,d), 8.98(1H,d), 8.69(2H,d), 8.15(3H,m), | 635.77 | 635.54 |
| | 7.98(3H,m), 7.82(5H,m), 7.70-7.58(9H,m), 7.49(1H,t) | | |
| 565 | 9.08(1H,d), 8.98(1H,d), 8.69(2H,d), 8.33(4H,m), | 559.67 | 559.20 |
| | 8.15(3H,m), 7.99(1H,d), 7.84(3H,m), 7.70-7.58(7H,m), | | |
| | 7.50(3H,m) | | |
| 566 | 9.18(1H,d), 9.08(1H,d), 8.98(1H,d), 8.78(1H,d), 8.69(4H,s), | 509.61 | 509.19 |
| | 8.55(1H,d), 8.17(1H,d), 8.11(1H,d), 7.99(1H,d), | | |
| | 7.83(1H,d), 7.74-7.61(7H,m), 7.37(1H,t), 7.23(1H,t), | | |
| | 6.88(1H,d) | | |
| 567 | 9.08(1H,d), 8.98(1H,d), 8.80(1H,d), 8.70(5H,m), | 609.73 | 609.22 |
| | 8.45(1H,d), 8.20(1H,d), 8.17(1H,d), 8.11(1H,d), | | |
| | 7.99(1H,d), 7.88(6H,m), 7.70-7.56(7H,m), 7.29(1H,d) | | |
| 568 | 9.08(1H,d), 8.98(1H,d), 8.69(2H,d), 8.56(1H,d), | 623.76 | 623.24 |
| | 8.17(1H,d), 8.11(1H,d), 7.99(3H,m), 7.83(4H,m), 7.70- | | |
| | 7.48(10H,m), 7.38(2H,m), 7.27(3H,m) | | |
| 569 | 9.08(1H,d), 8.98(1H,d), 8.69(2H,d), 8.48(1H,d), | 547.66 | 547.20 |
| | 8.28(3H,m), 8.17(1H,d), 8.11(1H,d), 7.99(1H,d), | | |
| | 7.84(5H,m), 7.70-7.61(6H,m), 7.54(1H,d), 7.21(1H,t), | | |
| | 6.86(1H,d) | | |
| 570 | 9.08(1H,d), 8.98(1H,d), 8.69(2H,d), 8.56(1H,d), | 575.71 | 575.24 |
| | 8.17(1H,d), 8.11(1H,d), 7.99(1H,d), 7.85-7.61(13H,m), | | |
| | 7.54(1H,d), 7.28(1H,t), 7.21(1H,t), 2.85(2H,q), 1.30(3H,t) | | |
| 571 | 9.08(1H,d), 8.98(1H,d), 8.36(1H,d), 8.17(1H,d), | 555.62 | 555.18 |
| | 8.11(1H,d), 7.99(1H,d), 7.96(2H,d), 7.83-7.61(11H,m), | | |
| | 7.51(6H,m) | | |
| 572 | 9.08(1H,d), 8.98(1H,d), 8.69(2H,d), 8.17(1H,d), | 686.82 | 686.25 |
| | 8.11(1H,d), 7.99(1H,d), 7.96(4H,m), 7.85-7.61(13H,m), | | |
| | 7.51(6H,m) | | |
| 573 | 9.08(114,d), 8.98(1H,d), 8.69(2H,d), 8.42(1H,d), | 783.97 | 783.29 |
| | 8.18(3H,m), 8.11-8.06(5H,m), 7.99(3H,d), 7.84(3H,m), | | |
| | 7.70-7.38(17H,m) | | |
| 574 | 9.08(1H,d), 8.98(1H,d), 8.69(2H,d), 8.21(4H,m), | 683.85 | 683.26 |
| | 8.17(1H,d), 8.11(1H,d), 7.99(1H,d), 7.83(1H,d), 7.75- | | |
| | 7.61(8H,m), 7.49(2H,t), 7.39(5H,m), 7.25(6H,m) | | |
| 575 | 9.08(1H,d), 8.98(1H,d), 8.69(2H,d), 8.17(1H,d), | 583.73 | 583.23 |
| | 8.11(1H,d), 8.04(3H,s), 7.99(1H,d), 7.85(3H,m), 7.75- | | |
| | 7.61(10H,m), 7.49(4H,m), 7.41(2H,m) | | |
| 576 | 9.08(1H,d), 8.98(1H,d),8.70(6H,m), 8.17(1H,d), | 585.71 | 585.22 |
| | 8.11(1H,d), 8.02(8H,m), 7.84(3H,m), 7.68-7.61(6H,m) | | |
| 577 | 9.08(1H,d), 8.98(1H,d), 8.56(1H,d), 8.28(2H,m), 8.17- | 547.66 | 547.20 |
| | 8.11(4H,m), 7.99(1H,d), 7.81(4H,m), 7.70-7.50(10H,m), | | |
| | 7.28(1H,t) | | |
| 578 | 9.08(1H,d), 8.98(1H,d), 8.69(2H,d), 8.26(1H,s), 8.20- | 482.59 | 482.18 |
| | 8.06(4H,m), 7.99(1H,d), 7.83(1H,d), 7.71-7.58(8H,m), | | |
| | 7.25(2H,m) | | |
| 579 | 9.08(1H,d), 8.98(1H,d), 8.87(1H,d), 8.69(2H,d), | 482.59 | 482.18 |
| | 8.55(1H,d), 8.17(1H,d), 8.11(1H,d), 8.05(1H,d), | | |
| | 7.97(2H,m), 7.83(1H,d), 7.70-7.61(7H,m), 7.49(1H,t), | | |
| | 7.25(2H,m) | | |
| 580 | 9.08(1H,d), 8.98(1H,d), 8.70(5H, m), 8.39(1H,d), | 635.77 | 635.24 |
| | 8.17(1H,d), 8.11(1H,d), 7.97(2H,m), 7.85-7.79(7H,m), | | |
| | 7.70-7.61(6H,m), 7.46-7.41(4H,m) | | |
| 581 | 9.08(1H,d), 8.98(1H,d), 8.69(2H,m), 8.29(4H,m), | 584.72 | 584.23 |
| | 8.20(2H,s), 8.17(11-1,d), 8.11(1H,d), 7.99(1H,d), | | |
| | 7.84(3H,m), 7.70-7.49(12H,m) | | |
| 582 | 9.08(1H,d), 8.98(1H,d), 8.85(2H,d), 8.69(2H,d), | 684.84 | 684.26 |
| | 8.37(2H,d), 8.20(2H,s), 8.17(1H,d), 8.11(1H,d), 8.06- | | |
| | 7.99(7H,m), 7.84(3H,m), 7.70-7.59(10H,m) | | |
| 583 | 9.08(1H,d), 8.98(1H,d), 8.69(4H,s), 8.29(2H,d), 8.20(2H,s), | 584.72 | 584.23 |
| | 8.17(1H,d), 8.11(1H,d), 7.99(1H,d), 7.83(1H,d), 7.75- | | |
| | 7.49(14H,m) | | |
| 584 | 9.08(1H,d), 8.98(1H,d), 8.85(1H,m), 8.69(4H,s), | 685.84 | 684.26 |
| | 8.37(1H,d), 8.20(2H,s), 8.17(1H,d), 8.11-7.99(7H,m), | | |
| | 7.93(1H,d), 7.83(1H,d), 7.72-7.59(12H,m) | | |
| 585 | 9.08(1H,d), 8.98(1H,d), 8.69(2H,d), 8.36(4H,m), | 662.80 | 662.25 |
| | 8.17(1H,d), 8.11(1H,d), 7.98(3H,m), 7.84(3H,m), 7.70- | | |
| | 7.61(6H,m), 7.50(6H,m), 7.25(2H,d) | | |
| 586 | 9.08(1H,d), 8.98(1H,d), 8.69(2H,d), 8.17(1H,d), | 738.89 | 738.29 |
| | 8.11(1H,d), 7.98(7H,m), 7.83(1H,d), 7.75-7.61(10H,m), | | |
| | 7.49(4H,m), 7.41(2H,m), 7.25(2H,d) | | |
| 587 | 9.08(1H,d), 8.98(1H,d), 8.70(4H,m), 8.17(1H,d), | 740.87 | 740.27 |
| | 8.11(1H,d), 7.98(1H,m), 7.83(1H,d), 7.70-7.61(6H,m), | | |
| | 7.25(4H,m) | | |
| 588 | 9.08(1H,d), 8.98(1H,d), 8.69(2H,d), 8.55(2H,d), | 761.93 | 761.28 |
| | 8.18(5H,m), 8.11(1H,d), 7.97(3H,m), 7.84(3H,m), 7.70- | | |
| | 7.58(9H, ), 7.50(2H,t), 7.35(2H,t), 7.18(4H,m) | | |
| 589 | 9.08(1H,d), 8.98(1H,d), 8.55(2H,d), 8.30(2H,m), | 761.93 | 761.28 |
| | 8.17(1H,d), 8.11(1H,d), 7.99-7.89(9H,m), 7.77- | | |
| | 7.61(13H,m), 7.50(2H,m), 7.35(2H,t), 7.16(2H,t) | | |
| 590 | 9.08(1H,d), 8.98(1H,d), 8.69(2H,d), 8.18(2H,m), | 564.71 | 564.17 |
| | 8.11(1H,d), 8.02-7.96(4H,m), 7.84(3H,m), 7.70- | | |
| | 7.51(8H,m), 7.25(2H,d) | | |
| 591 | 9.08(1H,d), 8.98(1H,d), 8.69(2H,d), 8.17(1H,d), | 548.64 | 548.19 |
| | 8.11(1H,d), 7.97(3H,m), 7.84(3H,m), 7.74-7.61(8H,m), | | |
| | 7.38(2H,m), 7.25(2H,d) | | |
| 592 | 9.08(1H,d), 8.98(1H,d), 8.69(2H,d), 8.17(1H,d), 8.11- | 607.76 | 607.23 |
| | 8.06(3H,m), 7.98(4H,m), 7.84(3H,m), 7.70-7.55(11H,m), | | |
| | 7.40(2H,t) | | |
| 593 | 9.08(1H,d), 8.98(1H,d), 8.69(5H,m), 8.55(1H,d), | 597.72 | 597.72 |
| | 8.18(2H,m), 8.11(1H,d), 7.99-7.94(3H,m), 7.85(2H,m), | | |
| | 7.70-7.58(8H,m), 7.50(1H,t), 7.35(1H,t), 7.18(2H,m) | | |
| 594 | 9.08(1H,d), 8.98(1H,d), 8.74(2H,s), 8.69(2H,s), 8.55(1H,d), | 598.71 | 598.22 |
| | 8.18(2H,m), 8.11(1H,d), 7.99-7.94(4H,m), 7.83(1H,d), | | |
| 595 | 7.70-7.58(7H,m), 7.50(1H,t), 7.35(1H,t), 7.18(2H,m) | 631.71 | 631.21 |
| | 9.08(1H,d), 8.98(1H,d), 8.69(2H,m), 8.17(1H,d), | | |
| | 8.11(1H,d), 7.98(5H,m), 7.85-7.61(13H,m), 7.51(6H,m) | | |
| 596 | 9.08(1H,d), 8.98(1H,d), 8.69(2H,d), 8.43(1H,s), | 681.77 | 681.22 |
| | 8.17(2H,m), 8.11(1H,d), 8.00(3H,m), 7.85-7.61(13H,m), | | |
| | 7.53(7H,m), 7.38(1H,d) | | |
| 597 | 9.08 (1H,d), 8.98(1H,d), 8.68(2H,d),8.56(1H,d), | 456.55 | 456.16 |
| | 8.28(2H,m), 8.17(1H,d), 8.11(1H,d), 7.99(1H,d), 7.85- | | |
| | 7.50(18H,m), 7.28(1H,t) | | |
| 598 | 9.08(1H,d), 8.98(1H,d), 8.69(1H,d), 8.18(2H,), | 506.61 | 506.18 |
| | 8.11(1H,d), 8.02-7.96(4H,m), 7.84(3H,m), 7.70- | | |
| | 7.51(8H,m), 7.25(2H,d) | | |
| 599 | 9.08(2H,d), 8.69(2H,d), 8.19(3H,m), 7.94(1H,d), | 456.55 | 456.16 |
| | 7.85(7H,m), 7.70(3H,m), 7.62(2H,t) | | |
| 600 | 9.08(2H,d), 8.91(1H,s), 8.39(1H,d), 8.19(3H,m), 7.99- | 506.61 | 506.18 |
| | 7.91(3H,m), 7.85(5H,m), 7.70(3H,m), 7.62(2H,t), | | |
| | 7.55(1H,s), 7.38(1H,d) | | |
| 601 | δ = 9.08(1H,d), 8.98(1H,d), 8.43(2H,s), 8.17(1H,d), | 507.64 | 507.20 |
| | 8.11(1H,d), 8.04(1H,s), 7.99(1H,d), 7.83(1H,d), 7.75- | | |
| | 7.61(10H,m), 7.49-7.41(6H,m) | | |
| 602 | δ = 9.08(1H,d), 8.97(3H,m), 8.50(2H,d), 8.43(2H,s), | 607.76 | 607.23 |
| | 8.19(3H,m), 8.10(3H,m), 8.04(1H,s), 7.99(1H,d), 7.83- | | |
| | 7.61(9H,m), 7.52(2H,m), 7.39(2H,m) | | |
| 603 | δ = 9.08(1H,d), 8.98(1H,d), 8.43(1H,s), 8.17-7.98(10H,m), | 687.80 | 687.22 |
| | 7.83(1H,d), 7.70 = −7.51(10H,m), 7.35(2H,m), 7.31(2H,m) | | |
| 604 | δ = 9.08(1H,d), 8.98(1H,d), 8.56(4H,s), 8.18(3H,m), | 685.83 | 685.25 |
| | 8.11(1H,d), 7.99(1H,d), 7.94(2H,m), 7.83(1H,d), 7.70- | | |
| | 7.58(9H,m), 7.50(2H,t), 7.35(2H,t), 7.18(4H,m) | | |
| 605 | δ = 9.19(1H,s) 8.98(1H,d), 8.45(4H,m), 8.17-8.11(7H,m), | 719.92 | 719.17 |
| | 8.04(1H,s), 7.99(2H,m), 7.93(2H,m), 7.83(1H,d), 7.70- | | |
| | 7.49(10H,m) | | |
| 606 | δ = 9.08(1H,d), 8.98(1H,d), 8.82(3H,s), 8.80(2H,d), | 711.83 | 711.24 |
| | 8.71(2H,d), 8.45(2H,d), 8.19(3H,m), 8.11(1H,d), | | |
| | 7.99(2H,d), 7.90(2H,d), 7.83(1H,d), 7.70-7.56(8H,m), | | |
| | 7.29(2H,d) | | |
| 607 | δ = 9.08(1H,d), 8.98(1H,d), 8.56(2H,d), 8.43(2H,s), | 892.08 | 891.34 |
| | 8.28(4H,m), 8.17(1H,d), 8.11(1H,d), 8.04(1H,s), | | |
| | 7.99(1H,d), 7.83-7.77(10H,m), 7.68-7.61(7H,m), 7.53- | | |
| | 7.50(8H,m), 7.28(2H,t) | | |
| 608 | δ = 9.08(1H,d), 8.98(1H,d), 8.84(2H,d), 8.43(2H,s), | 707.88 | 707.26 |
| | 8.27(2H,d), 8.17(1H,d), 8.11(1H,d), 8.05(3H,s), 7.99(1H,d), | | |
| | 7.90(2H,d), 7.83(1H,d), 7.70-7.61(14H,m) | | |
| 609 | δ = 9.08(1H,d), 8.98(1H,d), 8.43(2H,s), 8.17(1H,d), | 984.21 | 983.36 |
| | 8.11(1H,d), 8.04(1H,s), 7.99(1H,d), 7.90-7.78(8H,m), 7.70- | | |
| | 7.55(10H,m), 7.47-7.28(17H,m) | | |

Further, FIG. 4 to FIG. 31 are graphs each illustrating a PL (Photoluminescence) or LTPL (Low Temperature Photoluminescence) emission/absorption spectrum of a compound in a specific UV wavelength region.

PL was measured at room temperature using an LS55 luminescent spectrometer manufactured by Perkin Elmer, and LTPL was measured using an F7000 luminescent spectrometer manufactured by HITACHI, and analyzed using liquid nitrogen under low-temperature conditions of −196° C. (77K).

FIG. 4 is a graph illustrating a PL spectrum of a compound 16 at a wavelength of 302 nm.

FIG. 5 is a graph illustrating an LTPL spectrum of the compound 16 at a wavelength of 382 nm.

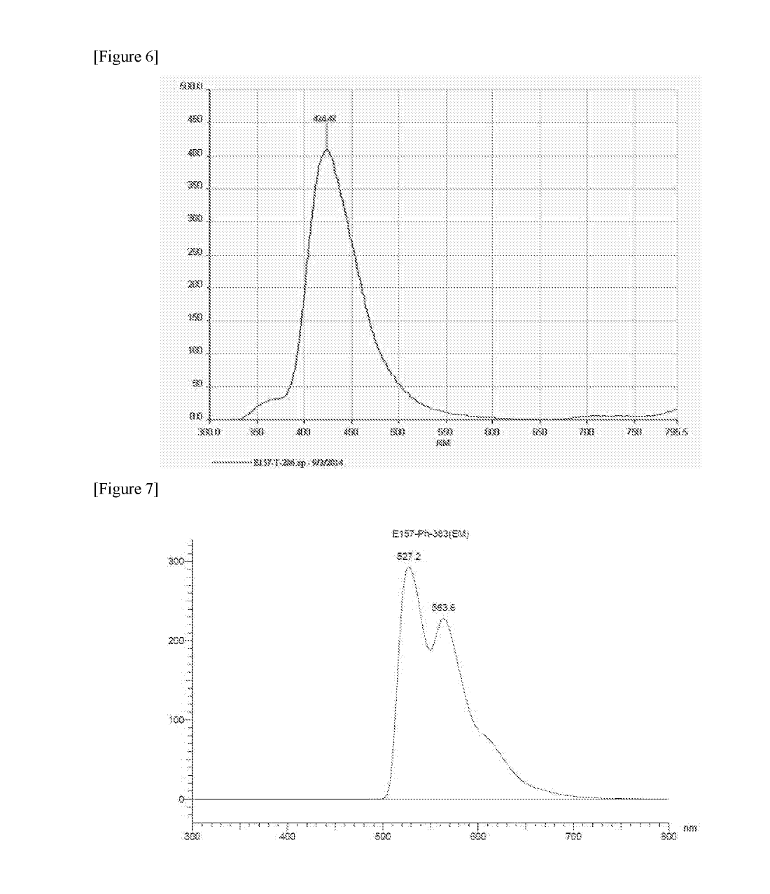

FIG. 6 is a graph illustrating a PL spectrum of a compound 18 at a wavelength of 286 nm.

FIG. 7 is a graph illustrating an LTPL spectrum of the compound 18 at a wavelength of 383 nm.

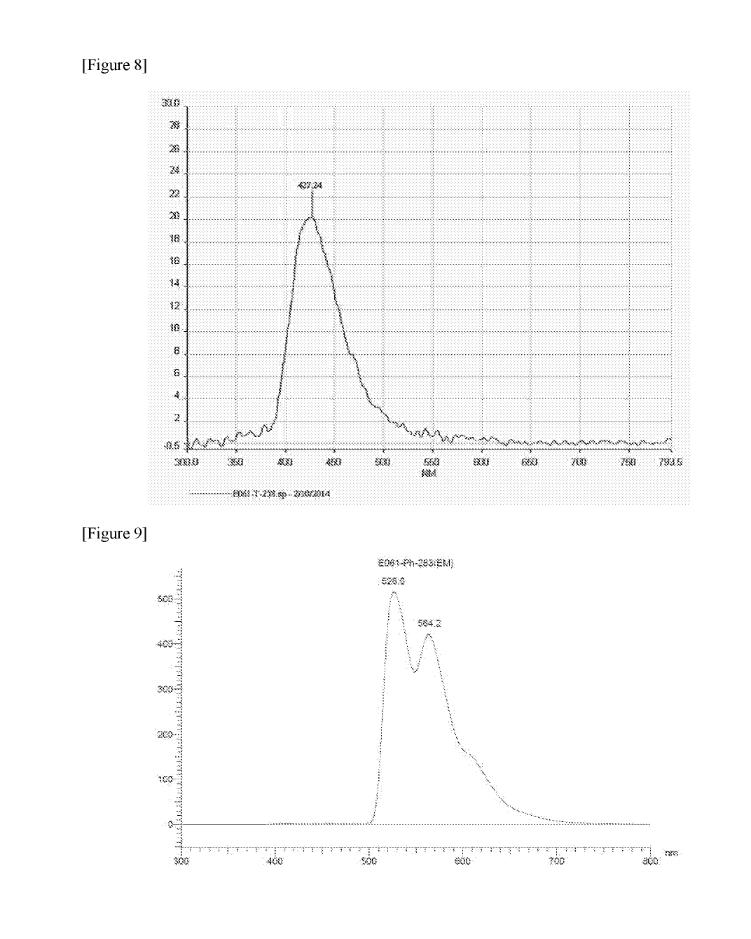

FIG. 8 is a graph illustrating a PL spectrum of a compound 22 at a wavelength of 258 nm.

FIG. 9 is a graph illustrating an LTPL spectrum of the compound 22 at a wavelength of 283 nm.

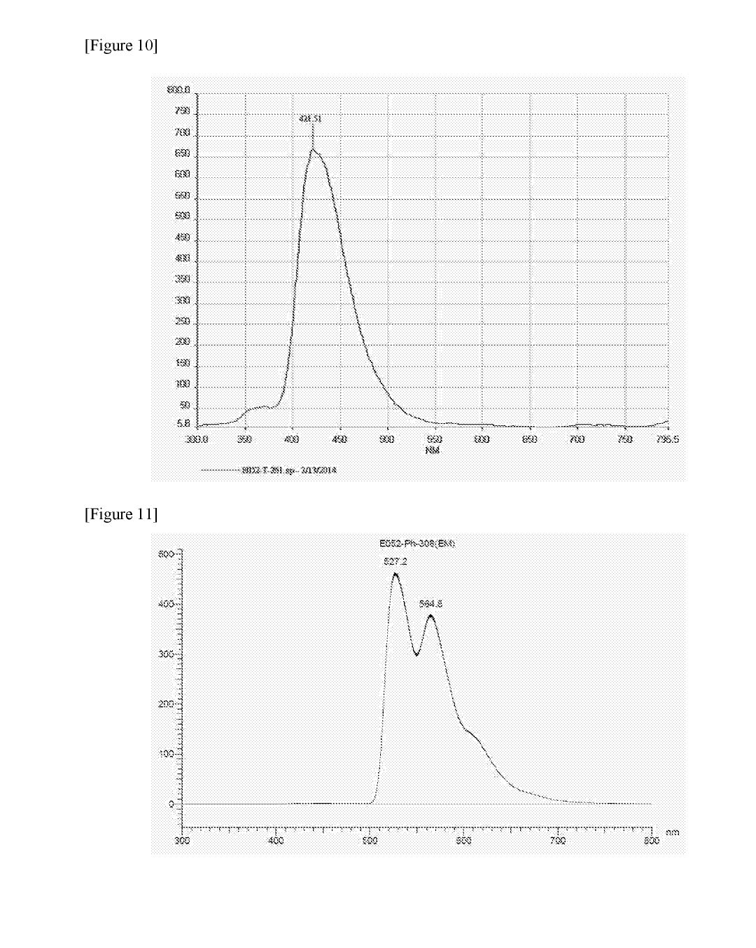

FIG. 10 is a graph illustrating a PL spectrum of a compound 37 at a wavelength of 261 nm.

FIG. 11 is a graph illustrating an LTPL spectrum of the compound 37 at a wavelength of 308 nm.

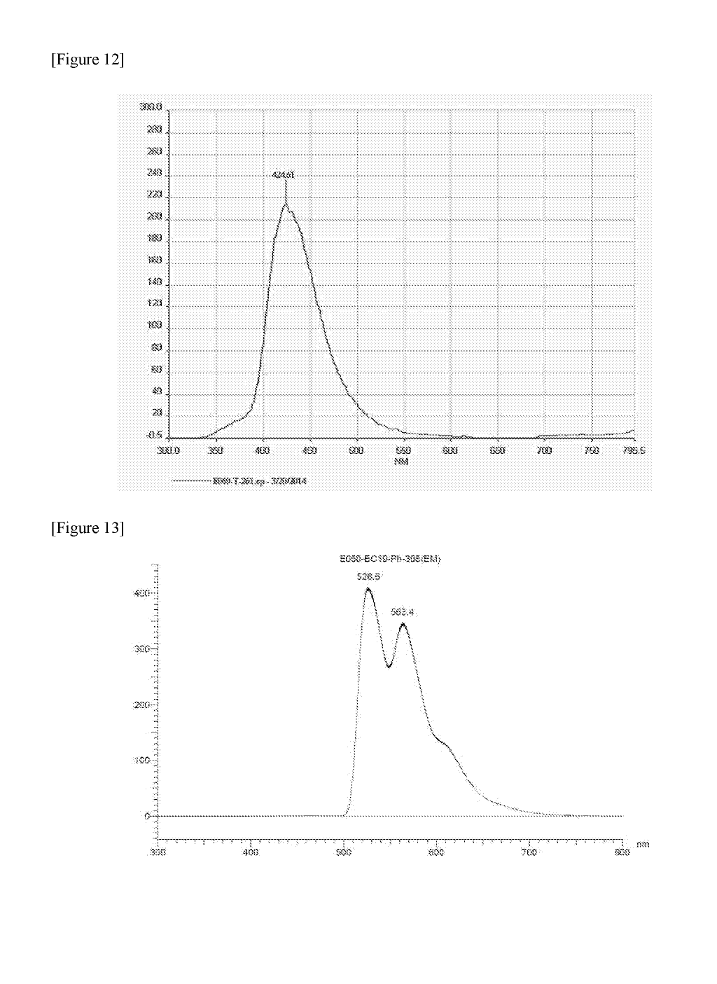

FIG. 12 is a graph illustrating a PL spectrum of a compound 41 at a wavelength of 261 nm.

FIG. 13 is a graph illustrating an LTPL spectrum of the compound 41 at a wavelength of 308 nm.

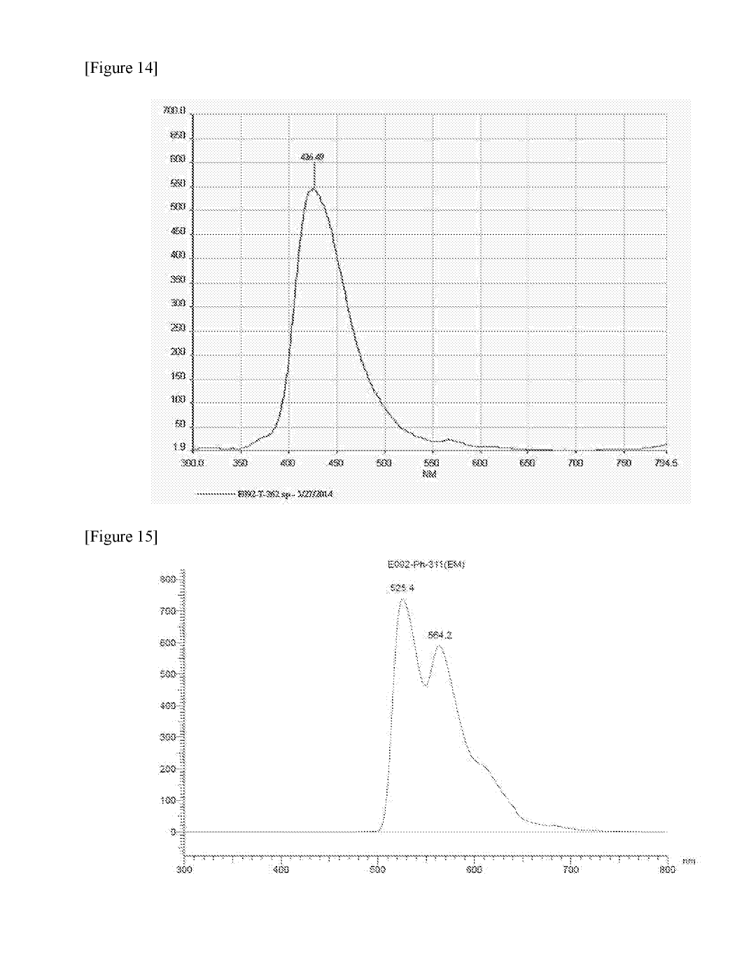

FIG. 14 is a graph illustrating a PL spectrum of a compound 43 at a wavelength of 262 nm.

FIG. 15 is a graph illustrating an LTPL spectrum of the compound 43 at a wavelength of 311 nm.

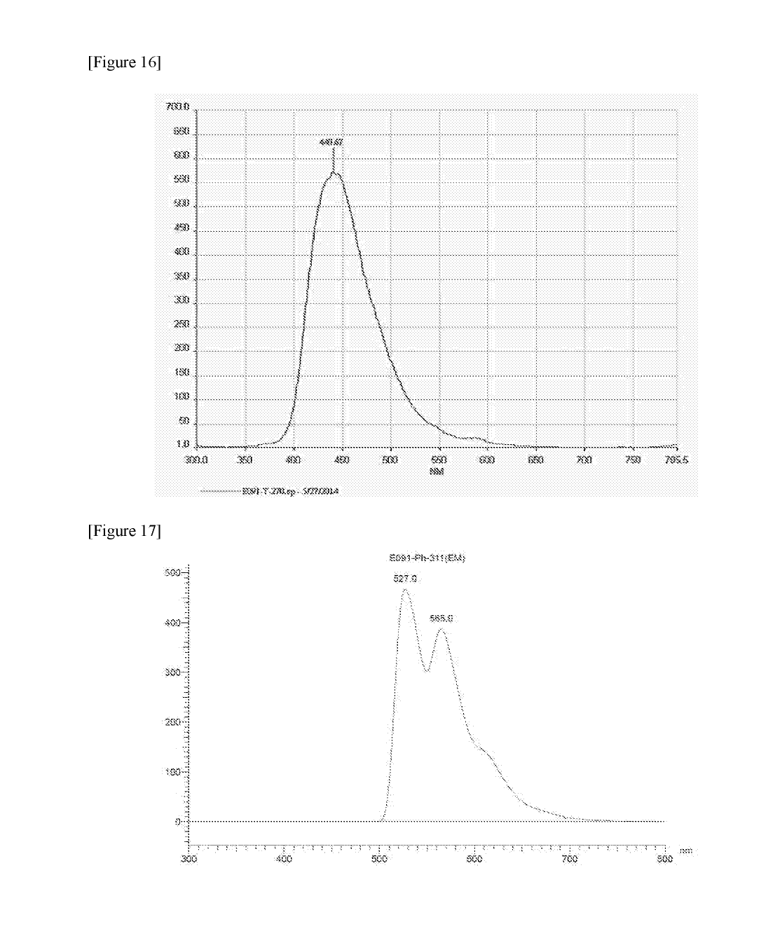

FIG. 16 is a graph illustrating a PL spectrum of a compound 44 at a wavelength of 270 nm.

FIG. 17 is a graph illustrating an LTPL spectrum of the compound 44 at a wavelength of 311 nm.

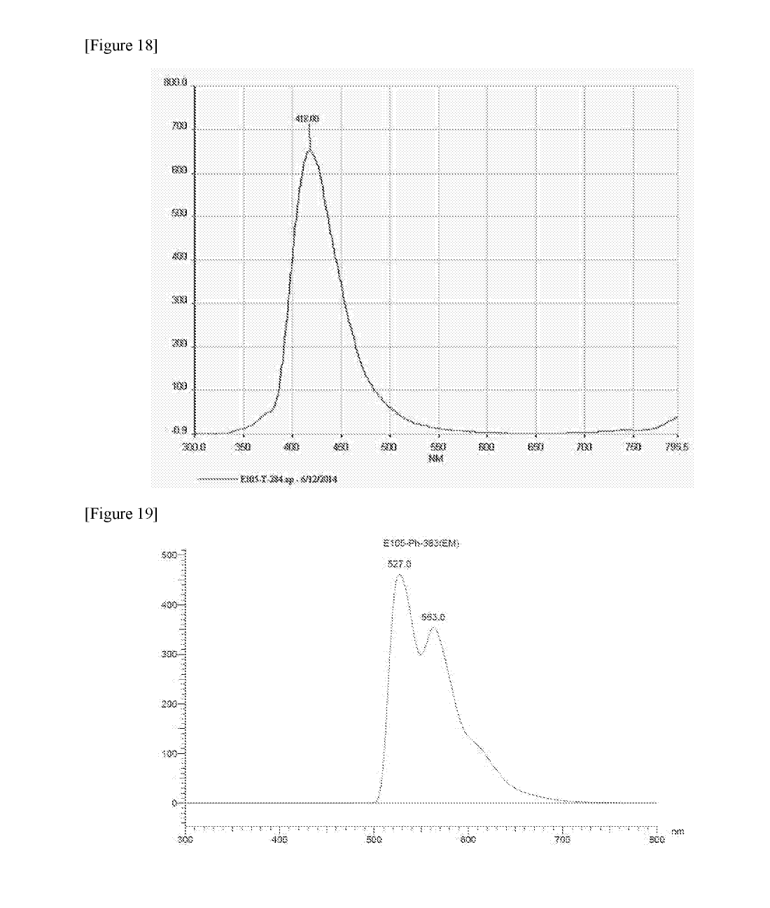

FIG. 18 is a graph illustrating a PL spectrum of a compound 73 at a wavelength of 284 nm.

FIG. 19 is a graph illustrating an LTPL spectrum of the compound 73 at a wavelength of 383 nm.

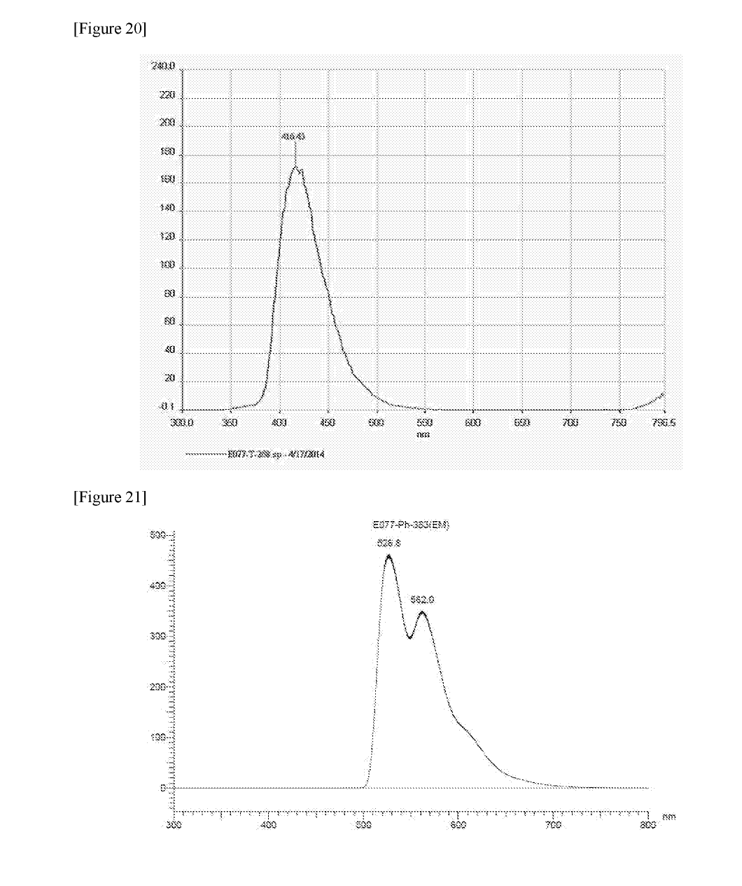

FIG. 20 is a graph illustrating a PL spectrum of a compound 92 at a wavelength of 268 nm.

FIG. 21 is a graph illustrating an LTPL spectrum of the compound 92 at a wavelength of 383 nm.

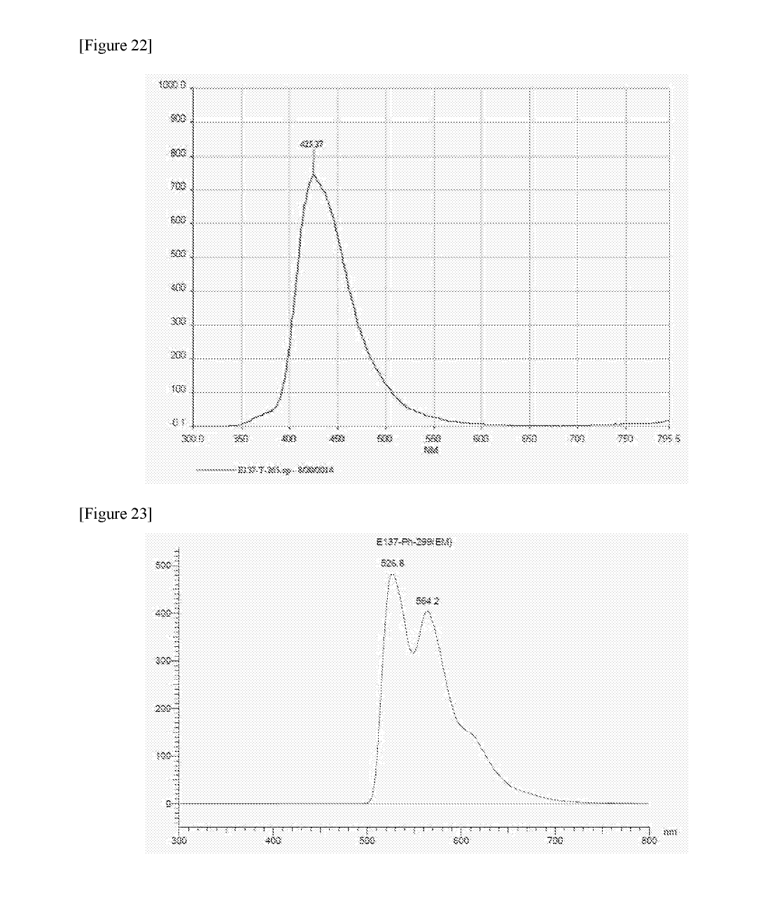

FIG. 22 is a graph illustrating a PL spectrum of a compound 294 at a wavelength of 265 nm.

FIG. 23 is a graph illustrating an LTPL spectrum of the compound 294 at a wavelength of 299 nm.

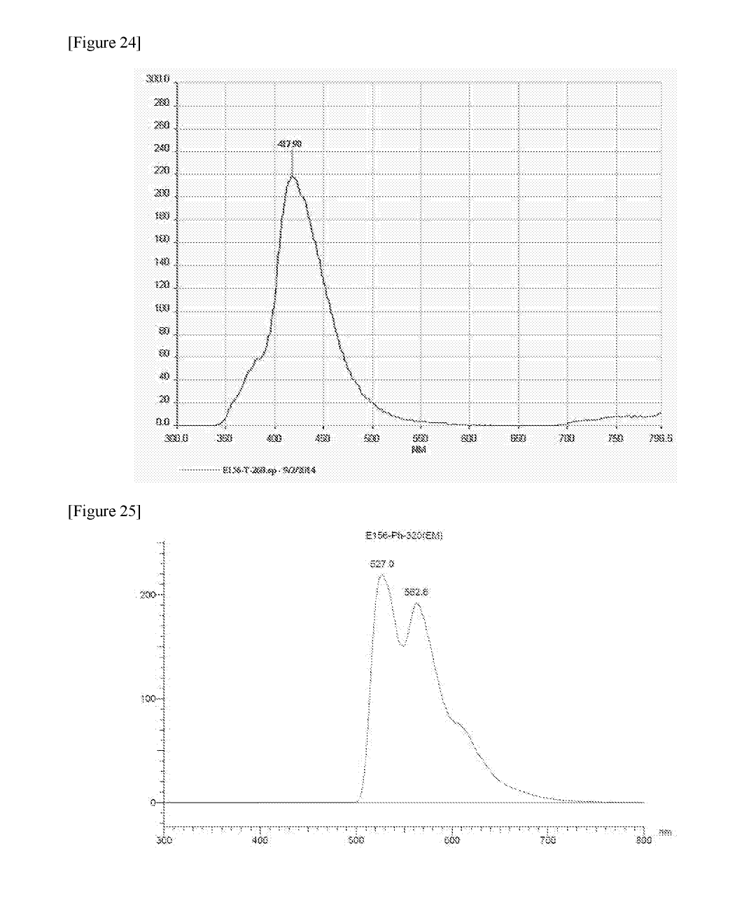

FIG. 24 is a graph illustrating a PL spectrum of a compound 324 at a wavelength of 260 nm.

FIG. 25 is a graph illustrating an LTPL spectrum of the compound 324 at a wavelength of 320 nm.

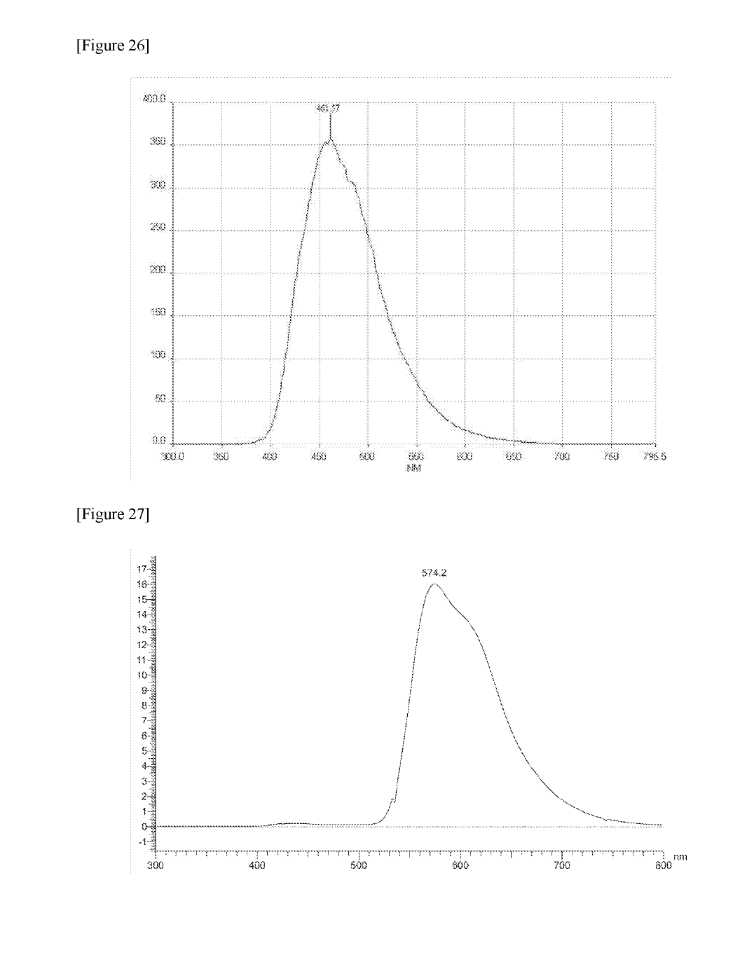

FIG. 26 is a graph illustrating a PL spectrum of a compound 560 at a wavelength of 273 nm.

FIG. 27 is a graph illustrating an LTPL spectrum of the compound 560 at a wavelength of 353 nm.

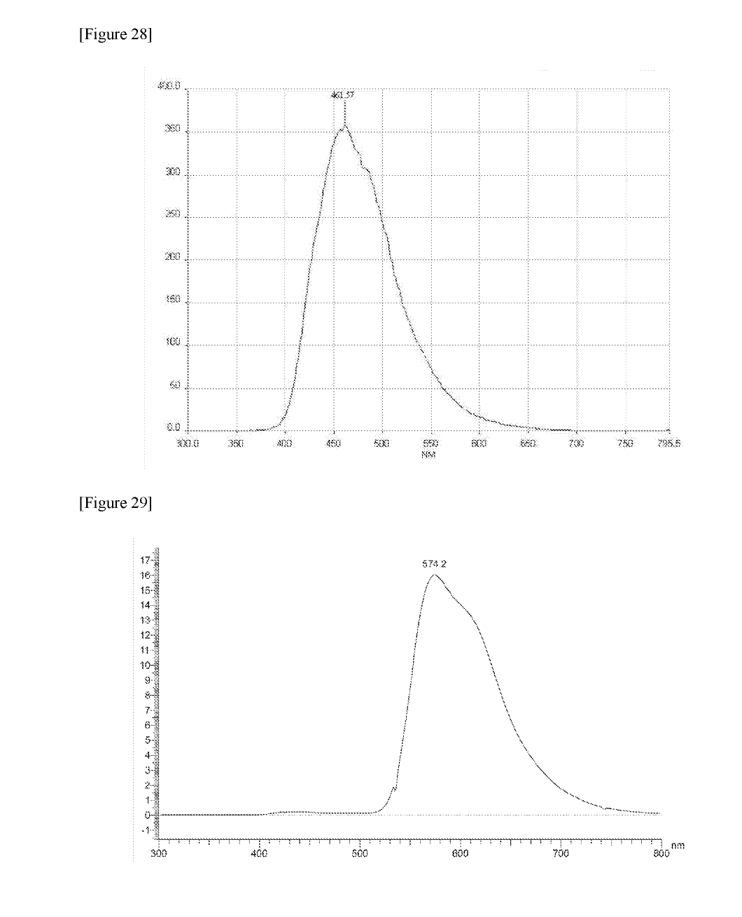

FIG. 28 is a graph illustrating a PL spectrum of a compound 563 at a wavelength of 273 nm.

FIG. 29 is a graph illustrating an LTPL spectrum of the compound 563 at a wavelength of 353 nm.

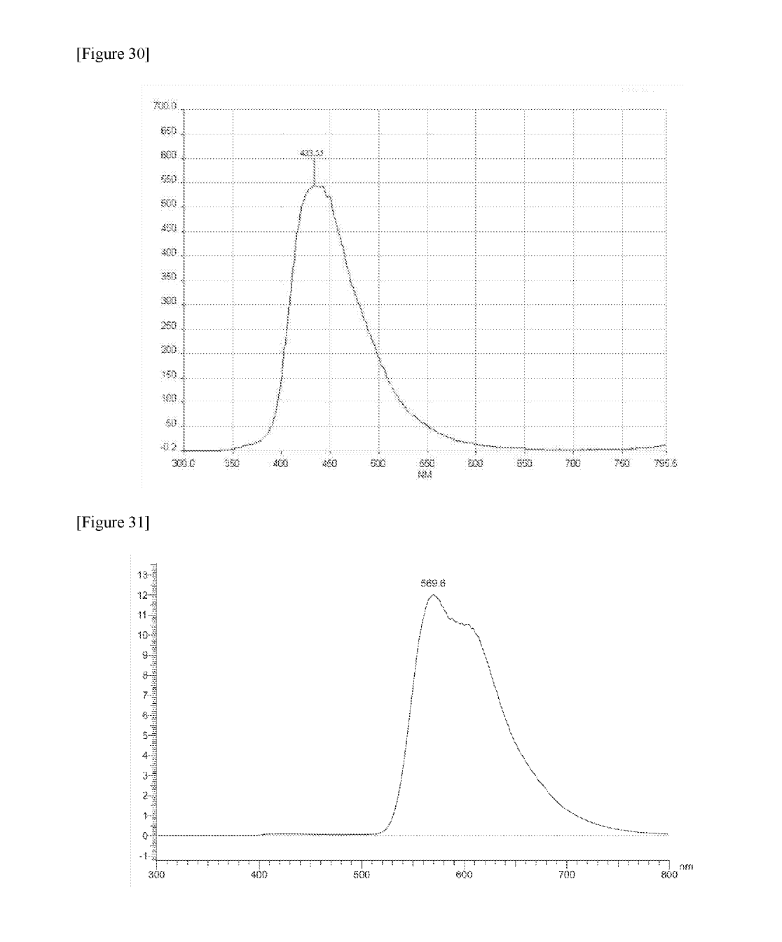

FIG. 30 is a graph illustrating a PL spectrum of a compound 568 at a wavelength of 253 nm.

FIG. 31 is a graph illustrating an LTPL spectrum of the compound 568 at a wavelength of 348 nm.

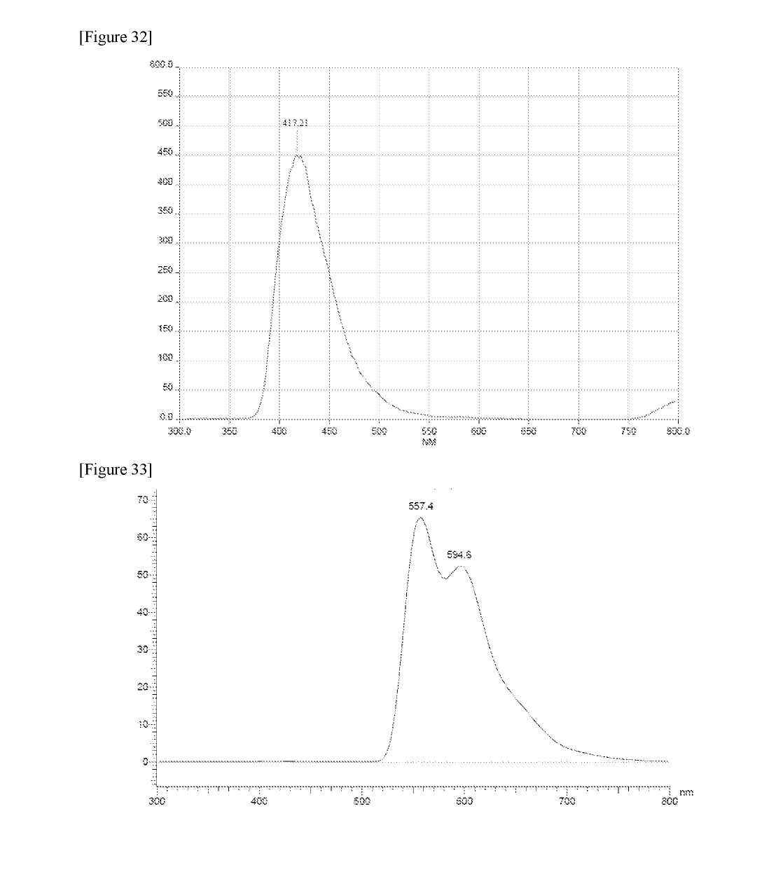

FIG. 32 is a graph illustrating a PL spectrum of a compound 603.

FIG. 33 is a graph illustrating an LTPL spectrum of the compound 603.

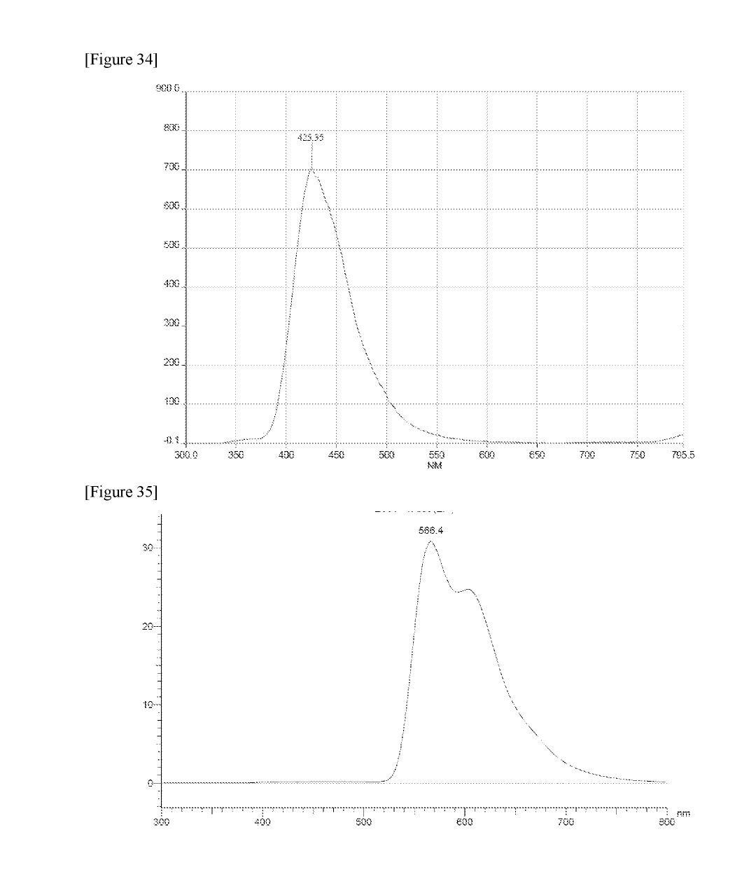

FIG. 34 is a graph illustrating a PL spectrum of a compound 604.

FIG. 35 is a graph illustrating an LTPL spectrum of the compound 604.

In the PL/LTPL graphs as illustrated in FIG. 4 to FIG. 35, the y-axis represents intensity and the x-axis represents a wavelength (unit: nm).

### Comparative Example 1

### Manufacturing of Organic Light Emitting Device

An organic electroluminescent device was manufactured by the following method.

A glass substrate coated with an ITO thin film to a thickness of 1500 Å was ultrasonic cleaned using distilled water. After cleaning with distilled water, the substrate was ultrasonic cleaned using solvents such as acetone, ethanol, isopropyl alcohol, and the like and dried, and then UVO-treated for 5 minutes using UV in a UV cleaner. Then, the substrate was transferred to a plasma cleaner (PT) and plasma-treated in a vacuum to remove ITO work function and a remaining film. Then, the substrate was transferred to a thermal deposition apparatus for depositing an organic substance.

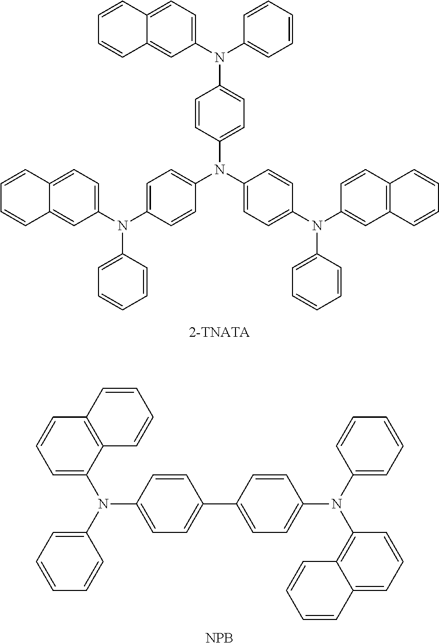 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(N(c2ccc(-c3ccc(N(c4ccccc4)c4cccc5ccccc45)cc3)cc2)c2cccc3ccccc23)cc1 ; "NPB"]

On the ITO transparent electrode (anode) prepared as described above, 4,4′,4″-tris(N,N-(2-naphthyl)-phenylamino)triphenyl amine (2-TNATA) was vacuum-deposited to a thickness of 600 Å so as to form a hole injection layer. Then, N, N′-bis(a-naphthyl)-N,N-diphenyl-4,4′-diamine (NPB) was vacuum-deposited to a thickness of 300 Å so as to form a hole transport layer.

After the hole injection layer and the hole transport layer as common layers are formed, a light emitting layer was vacuum-deposited thereon as described below. The light emitting layer used CBP as a host and Ir(ppy)₃ as a green phosphorescent dopant. Then, Bebq₂ was deposited as an electron transport layer to a thickness of 200 Å, and on the electron transport layer, lithium fluoride (LiF) was deposited as an electron injection layer to a thickness of 10 Å. Then, on the electron injection layer, aluminum (Al) cathode was deposited to a thickness of 1200 Å so as to form a cathode thereby manufacturing an organic electroluminescent device.

Meanwhile, each of all the organic compounds necessary for manufacturing of an OLED device was vacuumed, sublimed, and purified under 10⁻⁶ to 10⁻⁸ torr, and used for manufacturing OLED.

### Example 1

### Manufacturing of Organic Light Emitting Device

An organic electroluminescent device was manufactured in the same manner as the comparative example 1 except that the compounds 1 to 7 and 10 and 11 synthesized in the preparation examples 1 to 7 and 10 and 11 were used instead of Bebq₂ used in forming the electron transport layer in the comparative example 1.

### Experimental Example 1-1

### Driving Voltage and Efficiency of Organic Electroluminescent Device

Driving voltage and efficiency of the organic electroluminescent devices respectively manufactured in the above-described comparative example 1 and example 1 were measured at a luminescent brightness 6000 cd/m², and the results thereof were as listed in the following Table 3.

TABLE 3

| | Electron transport layer | Driving | Efficiency |
|---|---|---|---|
| | material | voltage (V) | (cd/A) |
| Preparation | 2 | 5.3 | 44 |
| Example 1 | | | |
| Preparation | 18 | 5.8 | 55 |
| Example 2 | | | |
| Preparation | 22 | 5.0 | 51 |
| Example 3 | | | |
| Preparation | 37 | 5.4 | 47 |
| Example 4 | | | |
| Preparation | 63 | 5.3 | 46 |
| Example 5 | | | |
| Preparation | 73 | 5.7 | 56 |
| Example 6 | | | |
| Preparation | 92 | 5.2 | 48 |
| Example 7 | | | |
| Preparation | 181 | 5.4 | 43 |
| Example 10 | | | |
| Preparation | 209 | 5.3 | 41 |
| Example 11 | | | |
| Comparative | Bebq₂ | 6.6 | 35 |
| Example 1 | | | |

According to the experimental results, it is exhibited that the organic electroluminescent devices using the compounds of the preparation examples 1 to 7 and 10 and 11 of the present invention have a low driving voltage and a high luminescent efficiency, as compared with the organic electroluminescent device of the comparative example 1 using conventional Bebq₂. Accordingly, it can be seen that since the compound according to the present invention is used as a material of an organic electroluminescent device, a driving voltage and a luminescent efficiency of the device can be improved.

### Experimental Example 1-2

### Life Span of Organic Electroluminescent Device

With respect to the organic electroluminescent devices respectively manufactured in the preparation examples 1 to 7 and 10 and 11, as a driving time of an organic electroluminescent device passes at 2000 cd/m², an average time for brightness to be decreased to 90% of an initial brightness when driving of the device is started was as listed in the following Table 4.

TABLE 4

| | Electron transport layer | T = |
|---|---|---|
| | material | 90%/hr |
| Preparation Example 1 | 2 | 48 |
| Preparation Example 2 | 18 | 58 |
| Preparation Example 3 | 22 | 50 |
| Preparation Example 4 | 37 | 46 |
| Preparation Example 5 | 63 | 47 |
| Preparation Example 6 | 73 | 56 |
| Preparation Example 7 | 92 | 49 |
| Preparation Example 10 | 181 | 48 |
| Preparation Example 11 | 209 | 50 |
| Comparative Example 1 | Bebq₂ | 40 |

According to the experimental results, it is exhibited that the organic electroluminescent devices using the compounds of the preparation examples 1 to 7 and 10 and 11 of the present invention have an excellent life span, as compared with the organic electroluminescent device of the comparative example 1 using conventional Bebq₂.

### Example 2

### Manufacturing of Organic Light Emitting Device

An organic electroluminescent device was manufactured in the same manner as the comparative example 1 except that the compounds 8 and 9 synthesized in the preparation examples 8 and 9 were used instead of CBP used in forming the light emitting layer in the comparative example 1.

### Experimental Example 2-1

### Driving Voltage and Efficiency of Organic Electroluminescent Device

Driving voltage and efficiency of the organic electroluminescent device manufactured in the comparative example 1 or the example 2 were measured at a luminescent brightness 6000 cd/m², and the results thereof were as listed in the following Table 5.

TABLE 5

| | Host material | Driving voltage (V) | Efficiency (cd/A) |
|---|---|---|---|
| Preparation | 111 | 4.7 | 42 |
| Example 8 | | | |
| Preparation | 130 | 4.8 | 40 |
| Example 9 | | | |
| Comparative | CBP | 6.6 | 35 |
| Example 1 | | | |

According to the experimental results, it is exhibited that the organic electroluminescent devices using the compounds of the preparation examples 8 and 9 of the present invention have a low driving voltage and a high luminescent efficiency, as compared with the organic electroluminescent device of the comparative example 1 using conventional CBP. Accordingly, it can be seen that since the compound according to the present invention is used as a material of an organic electroluminescent device, a driving voltage and a luminescent efficiency of the device can be improved.

### Experimental Example 2-2

### Life Span of Organic Electroluminescent Device

With respect to the organic electroluminescent devices respectively manufactured in the preparation examples 8 and 9, as a driving time of an organic electroluminescent device passes at 2000 cd/m², an average time for brightness to be decreased to 90% of an initial brightness when driving of the device is started was as listed in the following Table 6.

TABLE 6

| | Host material | T = 90%/hr |
|---|---|---|
| Preparation Example 8 | Compound 111 | 54 |
| Preparation Example 9 | Compound 130 | 51 |
| Comparative Example 1 | CBP | 40 |

According to the experimental results, it is exhibited that the organic electroluminescent devices using the compounds of the preparation examples 8 and 9 of the present invention have an excellent life span, as compared with the organic electroluminescent device of the comparative example 1 using conventional CBP.

### Comparative Example 2

First, a transparent electrode ITO thin film obtained from an OLED glass (manufactured by Samsung Corning Co., Ltd.) was ultrasonic cleaned using trichloroethylene, acetone, ethanol, and distilled water in sequence for each 5 minutes, and was used after being put into isopropanol.

Then, an ITO substrate was installed in a vacuum deposition apparatus. Thereafter, within a vacuum chamber, 4,4′,4″-tris(N, N-(2-naphthyl)-phenylamino)triphenyl amine (2-TNATA) was vacuum-deposited to a thickness of 600 Å on the ITO so as to form a hole injection layer.

Then, N,N′-bis(a-naphthyl)-N,N′-diphenyl-4,4′-diamine (NPB) was vacuum-deposited to a thickness of 300 Å on the hole injection layer so as to form a hole transport layer.

Thereafter, a light emitting layer was vacuum-deposited to a thickness of 200 Å on the hole transport layer with a blue emission host material H1 and a blue emission dopant material D1 at a mixing ratio of 95:5.

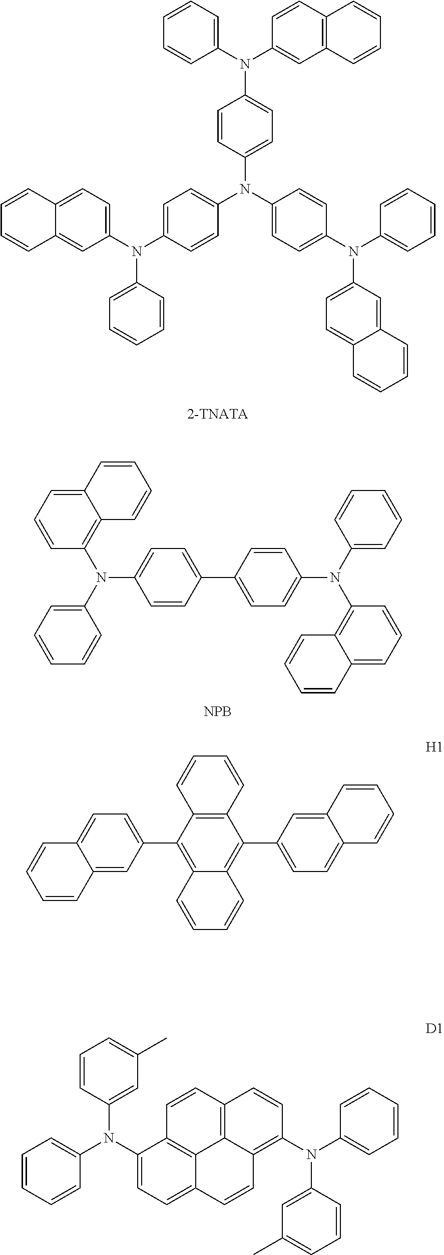

Then, a compound of the following structural formula E1 was deposited to a thickness of 300 Å on the light emitting layer so as to form an electron transport layer.

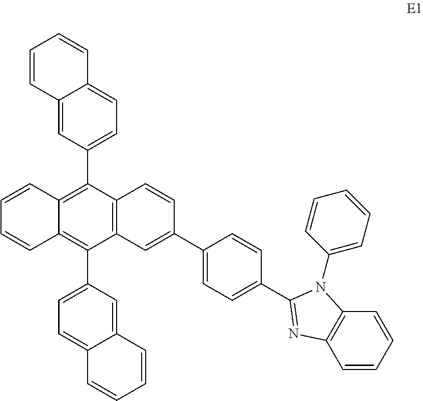 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

Thereafter, lithium fluoride (LiF) was deposited as an electron injection layer to a thickness of 10 Å on the electron transport layer, and Al was deposited as a cathode to a thickness of 1000 Å on the electron injection layer, thereby manufacturing an OLED device.

Meanwhile, each of all the organic compound materials necessary for manufacturing of an OLED device was vacuumed, sublimed, and purified under 10⁻⁶ to 10⁻⁸ torr, and used for manufacturing OLED.

### Example 3

An organic electroluminescent device was manufactured in the same manner as the comparative example 2 except that the compounds synthesized in the preparation examples 1 to 57 were used instead of E1 used for forming the electron transport layer in the comparative example 2.

### Experimental Example 3

### Evaluation of Organic Electroluminescent Device

Driving voltage, efficiency, color coordinate, and life span of the organic electroluminescent devices respectively manufactured in the above-described comparative example 2 and example 3 were measured at a luminescent brightness 700 cd/m², and the results thereof were as listed in the following Table 7.

Herein, the life span was measured using an M6000PMX manufactured by McScience Co., Ltd.

TABLE 7

| | | | | Color | | |
|---|---|---|---|---|---|---|
| Electron | Luminescent | Driving | | coordinate | | Life |
| transport layer | brightness | voltage | Efficiency | (x, y) | | span |
| material | (cd/m²) | (V) | (cd/A) | x | y | (T₅₀) |
| E1 | 700 | 4.70 | 4.50 | 0.150 | 0.180 | 330 |
| Compound 16 | 700 | 5.77 | 3.87 | 0.149 | 0.182 | 355 |
| Compound 18 | 700 | 4.48 | 4.48 | 0.153 | 0.186 | 960 |
| Compound 22 | 700 | 4.22 | 5.59 | 0.150 | 0.182 | 400 |
| Compound 37 | 700 | 4.54 | 5.31 | 0.149 | 0.182 | 456 |
| Compound 41 | 700 | 4.58 | 4.98 | 0.149 | 0.180 | 288 |
| Compound 43 | 700 | 4.48 | 4.94 | 0.148 | 0.182 | 192 |
| Compound 44 | 700 | 5.27 | 3.49 | 0.148 | 0.186 | 672 |
| Compound 45 | 700 | 4.60 | 4.71 | 0.153 | 0.167 | 384 |
| Compound 73 | 700 | 4.69 | 3.45 | 0.150 | 0.177 | 660 |
| Compound 92 | 700 | 5.50 | 4.45 | 0.148 | 0.182 | 380 |
| Compound 100 | 700 | 4.54 | 4.64 | 0.153 | 0.167 | 317 |
| Compound 185 | 700 | 4.48 | 4.86 | 0.153 | 0.170 | 317 |
| Compound 294 | 700 | 4.37 | 4.98 | 0.153 | 0.171 | 576 |
| Compound 324 | 700 | 4.43 | 4.94 | 0.153 | 0.169 | 480 |
| Compound 337 | 700 | 4.43 | 4.56 | 0.153 | 0.166 | 576 |
| Compound 376 | 700 | 4.55 | 4.64 | 0.153 | 0.175 | 499 |
| Compound 385 | 700 | 4.37 | 5.02 | 0.153 | 0.177 | 518 |
| Compound 388 | 700 | 4.45 | 4.86 | 0.153 | 0.180 | 499 |
| Compound 396 | 700 | 4.59 | 4.64 | 0.153 | 0.172 | 509 |
| Compound 404 | 700 | 4.70 | 4.53 | 0.153 | 0.167 | 490 |
| Compound 416 | 700 | 4.69 | 5.09 | 0.153 | 0.167 | 374 |
| Compound 421 | 700 | 4.60 | 4.48 | 0.153 | 0.169 | 432 |
| Compound 422 | 700 | 4.59 | 4.64 | 0.153 | 0.167 | 326 |
| Compound 457 | 700 | 4.53 | 4.79 | 0.153 | 0.168 | 432 |
| Compound 487 | 700 | 4.25 | 4.86 | 0.153 | 0.167 | 461 |
| Compound 489 | 700 | 4.60 | 4.48 | 0.153 | 0.165 | 326 |
| Compound 497 | 700 | 4.51 | 4.71 | 0.153 | 0.170 | 480 |
| Compound 517 | 700 | 4.37 | 5.24 | 0.153 | 0.168 | 653 |
| Compound 518 | 700 | 4.25 | 5.17 | 0.153 | 0.167 | 595 |
| Compound 519 | 700 | 4.13 | 4.71 | 0.153 | 0.169 | 634 |
| Compound 522 | 700 | 4.70 | 4.54 | 0.153 | 0.167 | 672 |
| Compound 530 | 700 | 4.61 | 4.48 | 0.153 | 0.167 | 768 |
| Compound 558 | 700 | 4.5 | 5.3 | 0.150 | 0.180 | 310 |
| Compound 559 | 700 | 4.4 | 5.0 | 0.150 | 0.180 | 340 |
| Compound 560 | 700 | 5.2 | 4.1 | 0.150 | 0.170 | 660 |
| Compound 561 | 700 | 4.7 | 5.1 | 0.150 | 0.180 | 350 |
| Compound 562 | 700 | 5.3 | 4.7 | 0.150 | 0.170 | 700 |
| Compound 563 | 700 | 4.8 | 4.6 | 0.150 | 0.160 | 800 |
| Compound 564 | 700 | 4.6 | 4.7 | 0.150 | 0.170 | 550 |
| Compound 565 | 700 | 4.8 | 4.4 | 0.150 | 0.190 | 480 |
| Compound 566 | 700 | 4.2 | 5.5 | 0.150 | 0.180 | 270 |
| Compound 567 | 700 | 4.7 | 4.5 | 0.150 | 0.170 | 330 |
| Compound 568 | 700 | 4.5 | 4.8 | 0.150 | 0.180 | 400 |
| Compound 569 | 700 | 4.3 | 5.2 | 0.150 | 0.180 | 450 |
| Compound 570 | 700 | 4.0 | 5.8 | 0.150 | 0.170 | 370 |
| Compound 571 | 700 | 5.3 | 4.5 | 0.150 | 0.170 | 900 |
| Compound 572 | 700 | 5.1 | 4.3 | 0.150 | 0.180 | 750 |
| Compound 577 | 700 | 4.6 | 5.1 | 0.150 | 0.170 | 950 |
| Compound 590 | 700 | 4.5 | 5.2 | 0.150 | 0.180 | 380 |
| Compound 597 | 700 | 4.3 | 5.9 | 0.150 | 0.180 | 750 |
| Compound 598 | 700 | 4.5 | 5.5 | 0.150 | 0.180 | 470 |
| Compound 599 | 700 | 4.6 | 5.0 | 0.150 | 0.170 | 430 |
| Compound 600 | 700 | 4.7 | 5.1 | 0.150 | 0.180 | 450 |
| Compound 603 | 700 | 4.2 | 5.1 | 0.150 | 0.170 | 450 |
| Compound 604 | 700 | 4.7 | 5.4 | 0.150 | 0.170 | 550 |
| Compound 605 | 700 | 4.5 | 4.6 | 0.150 | 0.170 | 700 |
| Compound 607 | 700 | 4.4 | 5.1 | 0.150 | 0.170 | 850 |
| Compound 608 | 700 | 4.5 | 5.0 | 0.150 | 0.170 | 400 |

As can be seen from the results listed in Table 7, each organic electroluminescent device using a compound according to the exemplary embodiment of the present application has a low driving voltage and a high luminescent efficiency as compared with the organic electroluminescent device of the comparative example 2 using E1 as an electron transport layer material. Further, it has an excellent durability, i.e., an excellent life span, as compared with the comparative example 2.

The exemplary embodiments have been explained above, but the present invention is not limited thereto and can be modified and changed in various ways within the scope of the accompanying claims and the detailed description of the invention, and all modifications and changes and their equivalents are included in the scope of the present invention.

1. A compound of the following chemical formula 1: 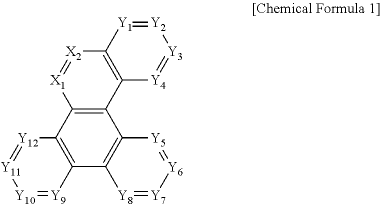
wherein in the chemical formula 1,
X₁ and X₂ are the same as or different from each other, and are each independently N or CR₁,
Y₁ to Y₁₂ are the same as or different from each other, and are each independently N or CR,
R and R₁ are the same as or different from each other, and are each independently one selected from the group consisting of hydrogen; halogen; linear or branched substituted or unsubstituted C₁ to C₆₀ alkyl; linear or branched substituted or unsubstituted C₂ to C₆₀ alkenyl; linear or branched substituted or unsubstituted C₂ to C₆₀ alkynyl; linear or branched substituted or unsubstituted C₁ to C₆₀ alkoxy; substituted or unsubstituted C₃ to C₆₀ monocyclic or polycyclic cycloalkyl; substituted or unsubstituted C₂ to C₆₀ monocyclic or polycyclic heterocycloalkyl; substituted or unsubstituted C₆ to C₆₀ monocyclic or polycyclic aryl; substituted or unsubstituted C₂ to C₆₀ monocyclic or polycyclic heteroaryl; —SiR₁₁R₁₂R₁₃; —P(═O)R₁₄R₁₅; and amine unsubstituted or substituted with C₁ to C₂₀ alkyl, substituted or unsubstituted C₆ to C₆₀ monocyclic or polycyclic aryl, or substituted or unsubstituted C₂ to C₆₀ monocyclic or polycyclic heteroaryl, or at least two adjacent Rs are bonded to each other to form a substituted or unsubstituted monocyclic or polycyclic aliphatic or aromatic hydrocarbon ring;
R₁₁ to R₁₅ are the same as or different from each other, and are each independently one selected from the group consisting of linear or branched substituted or unsubstituted C₁ to C₆₀ alkyl; substituted or unsubstituted C₃ to C₆₀ monocyclic or polycyclic cycloalkyl; substituted or unsubstituted C₆ to C₆₀ monocyclic or polycyclic aryl; and substituted or unsubstituted C₂ to C₆₀ monocyclic or polycyclic heteroaryl.

2. The compound of claim 1, wherein Formula 1 is represented by the following Formula 2, 3 or 9: 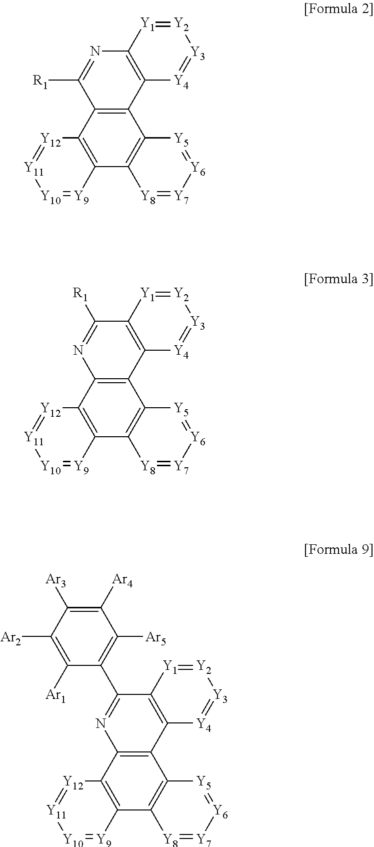
in Formulae 2, 3, and 9,
Y₁ to Y₁₂ and R and R₁ are the same as those defined in Formula 1, and
at least two of Ar₁ to Ar₅ are monocyclic or polycyclic substituted or unsubstituted C₆ to C₆₀ aryl; or monocyclic or polycyclic substituted or unsubstituted C₂ to C₆₀ heteroaryl, and the others are each independently hydrogen; deuterium; halogen; straight-chained or branched substituted or unsubstituted C₁ to C₆₀ alkyl; monocyclic or polycyclic substituted or unsubstituted C₆ to C₆₀ aryl; or monocyclic or polycyclic substituted or unsubstituted C₂ to C₆₀ heteroaryl.

3. The compound of claim 1, wherein all of the Y₁ to Y₄ are CR, or any one of the Y₁ to Y₄ is N and the others are CR, and
the R is the same as defined in the chemical formula 1.

4. The compound of claim 1, wherein all of the Y₅ to Y₈ are CR, or any one of the Y₅ to Y₈ is N and the others are CR, and
the R is the same as defined in the chemical formula 1.

5. The compound of claim 1, wherein all of the Y₉ to Y₁₂ are CR, or any one of the Y₉ to Y₁₂ is N and the others are CR, and
the R is the same as defined in the chemical formula 1.

6. The compound of claim 1, wherein the Y₁ to Y₁₂ are CR, and the R is the same as defined in the chemical formula 1.

7. The compound of claim 2, wherein the R₁ is -(L)m-(Z)n,
L is substituted or unsubstituted C₆ to C₆₀ monocyclic or polycyclic arylene; or substituted or unsubstituted C₂ to C₆₀ monocyclic or polycyclic heteroarylene,
Z is one selected from the group consisting of substituted or unsubstituted C₆ to C₆₀ monocyclic or polycyclic aryl; substituted or unsubstituted C₂ to C₆₀ monocyclic or polycyclic heteroaryl; —SiR₁₁R₁₂R₁₃; and —P(═O)R₁₄R₁₅,
R₁₁ to R₁₅ are the same as or different from each other, and are each independently one selected from the group consisting of linear or branched C₁ to C₆₀ alkyl; substituted or unsubstituted C₃ to C₆₀ monocyclic or polycyclic cycloalkyl; C₆ to C₆₀ monocyclic or polycyclic aryl; and C₂ to C₆₀ monocyclic or polycyclic heteroaryl,
m is an integer of 0 to 5,
n is an integer of 1 to 3, and
when m and n are independently integers of 2 or more, multiple L and Z are the same as or different from each other.

8. The compound of claim 2, wherein the R₁ is -(L)m-(Z)n,
L is one selected from the group consisting of C₆ to C₆₀ monocyclic or polycyclic arylene unsubstituted or substituted with halogen, linear or branched C₁ to C₆₀ alkyl, C₃ to C₆₀ monocyclic or polycyclic cycloalkyl, C₆ to C₆₀ monocyclic or polycyclic aryl, and C₂ to C₆₀ monocyclic or polycyclic heteroaryl; and C₂ to C₆₀ monocyclic or polycyclic heteroarylene unsubstituted or substituted with halogen, linear or branched C₁ to C₆₀ alkyl, C₃ to C₆₀ monocyclic or polycyclic cycloalkyl, C₆ to C₆₀ monocyclic or polycyclic aryl, and C₂ to C₆₀ monocyclic or polycyclic heteroaryl,
Z is one selected from the group consisting of C₆ to C₆₀ monocyclic or polycyclic aryl unsubstituted or substituted with halogen, linear or branched C₁ to C₆₀ alkyl, C₃ to C₆₀ monocyclic or polycyclic cycloalkyl, C₆ to C₆₀ monocyclic or polycyclic aryl, and C₂ to C₆₀ monocyclic or polycyclic heteroaryl; C₂ to C₆₀ monocyclic or polycyclic heteroaryl unsubstituted or substituted with halogen, linear or branched C₁ to C₆₀ alkyl, C₃ to C₆₀ monocyclic or polycyclic cycloalkyl, C₆ to C₆₀ monocyclic or polycyclic aryl, and C₂ to C₆₀ monocyclic or polycyclic heteroaryl; —SiR₁₁R₁₂R₁₃; and —P(═O)R₁₄R₁₅, and the R₁₁ to R₁₅ are the same as or different from each other, and are each independently one selected from the group consisting of hydrogen; linear or branched C₁ to C₆₀ alkyl unsubstituted or substituted with halogen, linear or branched C₁ to C₆₀ alkyl, C₃ to C₆₀ monocyclic or polycyclic cycloalkyl, C₆ to C₆₀ monocyclic or polycyclic aryl, and C₂ to C₆₀ monocyclic or polycyclic heteroaryl; C₆ to C₆₀ monocyclic or polycyclic aryl unsubstituted or substituted with halogen, linear or branched C₁ to C₆₀ alkyl, C₃ to C₆₀ monocyclic or polycyclic cycloalkyl, C₆ to C₆₀ monocyclic or polycyclic aryl, and C₂ to C₆₀ monocyclic or polycyclic heteroaryl; and C₂ to C₆₀ monocyclic or polycyclic heteroaryl unsubstituted or substituted with halogen, linear or branched C₁ to C₆₀ alkyl, C₃ to C₆₀ monocyclic or polycyclic cycloalkyl, C₆ to C₆₀ monocyclic or polycyclic aryl, and C₂ to C₆₀ monocyclic or polycyclic heteroaryl,
m is an integer of 0 to 5,
n is an integer of 1 to 3,
when m and n are independently integers of 2 or more, multiple L and Z are the same as or different from each other,
Y₁ to Y₁₂ are the same as or different from each other, and are each independently N or CR,
R is one selected from the group consisting of C₆ to C₆₀ monocyclic or polycyclic aryl unsubstituted or substituted with halogen, linear or branched C₁ to C₆₀ alkyl, C₃ to C₆₀ monocyclic or polycyclic cycloalkyl, C₆ to C₆₀ monocyclic or polycyclic aryl, and C₂ to C₆₀ monocyclic or polycyclic heteroaryl; C₂ to C₆₀ monocyclic or polycyclic heteroaryl unsubstituted or substituted with halogen, linear or branched C₁ to C₆₀ alkyl, C₃ to C₆₀ monocyclic or polycyclic cycloalkyl, C₆ to C₆₀ monocyclic or polycyclic aryl, and C₂ to C₆₀ monocyclic or polycyclic heteroaryl; —SiR₁₁R₁₂R₁₃; and —P(═O)R₁₄R₁₅, and the R₁₁ to R₁₅ are the same as or different from each other, and are each independently one selected from the group consisting of hydrogen; linear or branched alkyl unsubstituted or substituted with halogen, linear or branched C₁ to C₆₀ alkyl, C₃ to C₆₀ monocyclic or polycyclic cycloalkyl, C₆ to C₆₀ monocyclic or polycyclic aryl, and C₂ to C₆₀ monocyclic or polycyclic heteroaryl; C₆ to C₆₀ monocyclic or polycyclic aryl unsubstituted or substituted with halogen, linear or branched C₁ to C₆₀ alkyl, C₃ to C₆₀ monocyclic or polycyclic cycloalkyl, C₆ to C₆₀ monocyclic or polycyclic aryl, and C₂ to C₆₀ monocyclic or polycyclic heteroaryl; and C₂ to Coo monocyclic or polycyclic heteroaryl unsubstituted or substituted with halogen, linear or branched C₁ to C₆₀ alkyl, C₃ to C₆₀ monocyclic or polycyclic cycloalkyl, C₆ to C₆₀ monocyclic or polycyclic aryl, and C₂ to C₆₀ monocyclic or polycyclic heteroaryl, and
if two or more of the Y₁ to Y₁₂ are CR, multiple R are the same as or different from each other.

9. The compound of claim 7, wherein Z is substituted or unsubstituted phenyl, substituted or unsubstituted biphenyl, substituted or unsubstituted naphthyl, substituted or unsubstituted chrysenyl, substituted or unsubstituted pyrenyl, substituted or unsubstituted triphenylenyl, substituted or unsubstituted anthracenyl, substituted or unsubstituted phenanthrenyl, substituted or unsubstituted fluorenyl, substituted or unsubstituted benzo chrysenyl, or substituted or unsubstituted spirobifluorenyl.

10. The compound of claim 7, wherein Z is substituted or unsubstituted C₂ to C₆₀ monocyclic or polycyclic heteroaryl, and the heteroaryl includes at least one selected from N, O, S, Si, and Se as a heteroatom.

11. The compound of claim 7, wherein Z is 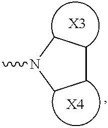
and X3 and X4 are substituted or unsubstituted C₆ to C₆₀ monocyclic or polycyclic aromatic hydrocarbon rings; or substituted or unsubstituted C₂ to C₆₀ monocyclic or polycyclic aromatic hetero rings.

12. The compound of claim 11, wherein 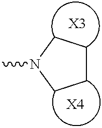
is represented by any one of the following structural formulas: 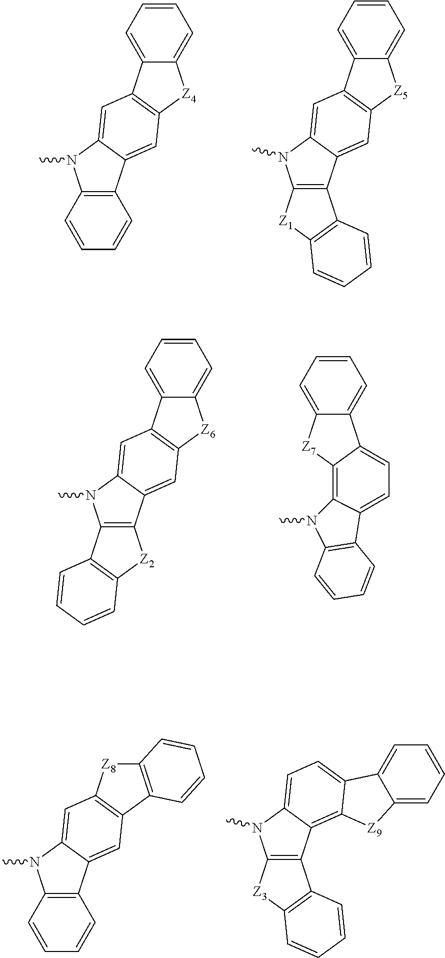
wherein in the structural formulas, Z₁ to Z₃ are the same as or different from each other, and are each independently S or O,
Z₄ to Z₉ are the same as or different from each other, and are each independently CR′ R″, NR′, S, or O,
R′ and R″ are the same as or different from each other, and are each independently hydrogen; linear or branched substituted or unsubstituted C₁ to C₆₀ alkyl; or substituted or unsubstituted C₆ to C₆₀ monocyclic or polycyclic aryl.

13. The compound of claim 1, wherein the chemical formula 1 is represented by the following chemical formula 4 or 5: 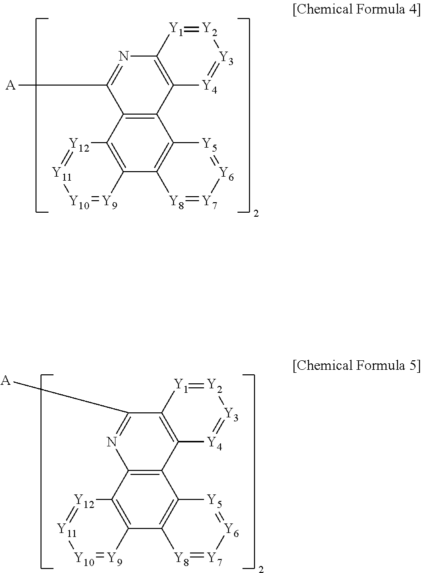
wherein in the chemical formulas 4 and 5,
A is directly bonded; or selected from the group consisting of linear or branched substituted or unsubstituted C₂ to C₆₀ alkylene, linear or branched substituted or unsubstituted C₂ to C₆₀ alkenylene, linear or branched substituted or unsubstituted C₂ to C₆₀ alkynylene, substituted or unsubstituted C₃ to C₆₀ monocyclic or polycyclic cycloalkylene, substituted or unsubstituted C₂ to C₆₀ monocyclic or polycyclic heterocycloalkylene, substituted or unsubstituted C₆ to C₆₀ monocyclic or polycyclic arylene, and substituted or unsubstituted C₂ to C₆₀ monocyclic or polycyclic heteroarylene, and
Y₁ to Y₁₂ are the same as defined in the chemical formula 1.

14. The compound of claim 13, wherein A is directly bonded; or selected from the group consisting of substituted or unsubstituted C₆ to C₆₀ monocyclic or polycyclic arylene, and substituted or unsubstituted C₂ to C₆₀ monocyclic or polycyclic heteroarylene.

15. The compound of claim 1, wherein the compound of the chemical formula 1 is selected from the following compounds:

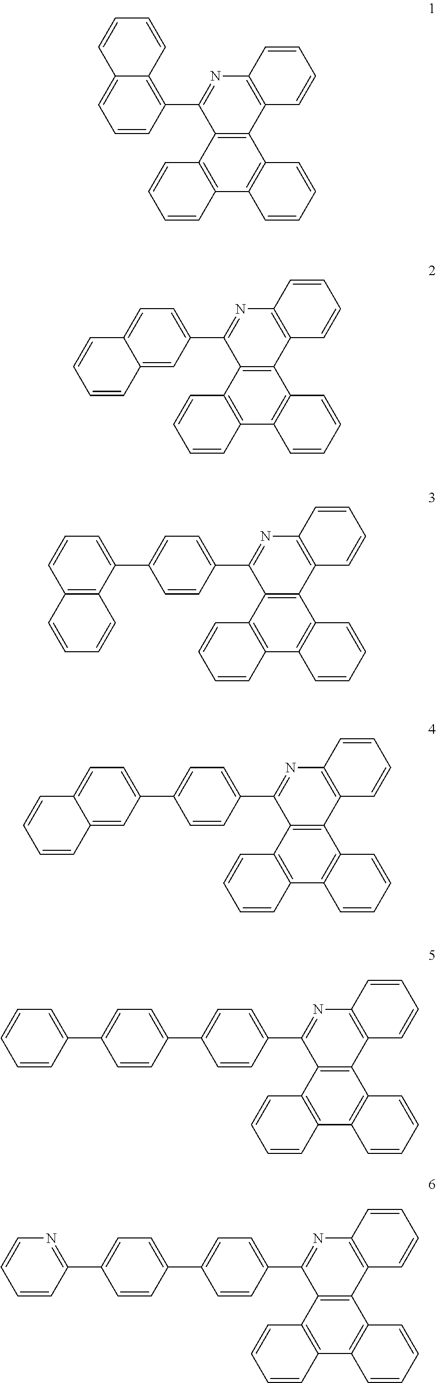 [SMILES: c1ccc2c(-c3nc4ccccc4c4c5ccccc5c5ccccc5c34)cccc2c1 ; c1ccc2cc(-c3nc4ccccc4c4c5ccccc5c5ccccc5c34)ccc2c1 ; c1ccc2c(-c3ccc(-c4nc5ccccc5c5c6ccccc6c6ccccc6c45)cc3)cccc2c1 ; c1ccc2cc(-c3ccc(-c4nc5ccccc5c5c6ccccc6c6ccccc6c45)cc3)ccc2c1 ; c1ccc(-c2ccc(-c3ccc(-c4nc5ccccc5c5c6ccccc6c6ccccc6c45)cc3)cc2)cc1 ; c1ccc(-c2ccc(-c3ccc(-c4nc5ccccc5c5c6ccccc6c6ccccc6c45)cc3)cc2)nc1]

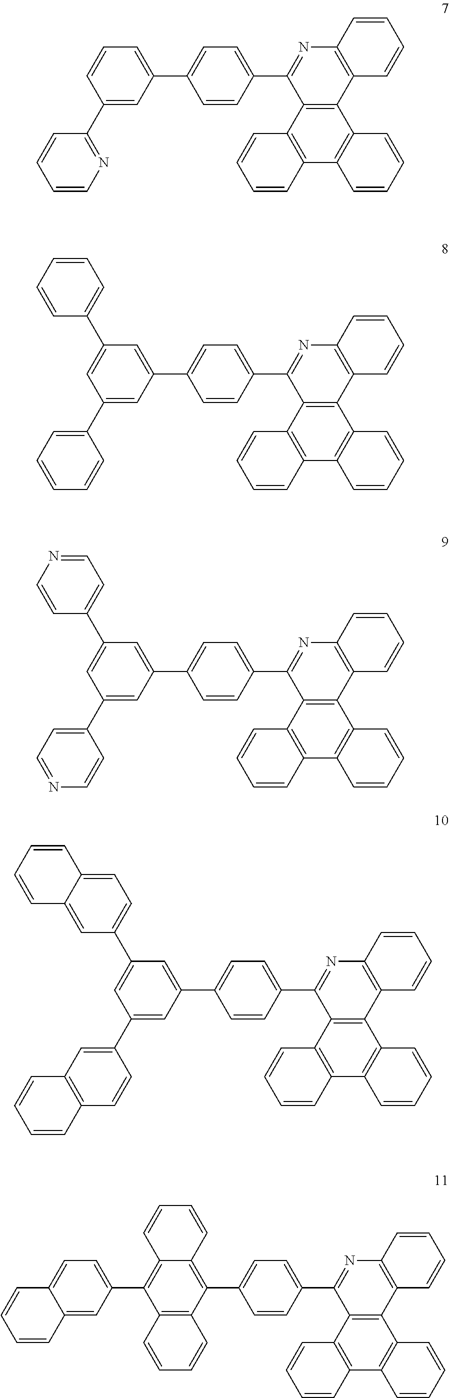 [SMILES: c1ccc(-c2cccc(-c3ccc(-c4nc5ccccc5c5c6ccccc6c6ccccc6c45)cc3)c2)nc1 ; c1ccc(-c2cc(-c3ccccc3)cc(-c3ccc(-c4nc5ccccc5c5c6ccccc6c6ccccc6c45)cc3)c2)cc1 ; c1ccc2c(c1)nc(-c1ccc(-c3cc(-c4ccncc4)cc(-c4ccncc4)c3)cc1)c1c3ccccc3c3ccccc3c21 ; c1ccc2cc(-c3cc(-c4ccc(-c5nc6ccccc6c6c7ccccc7c7ccccc7c56)cc4)cc(-c4ccc5ccccc5c4)c3)ccc2c1 ; c1ccc2cc(-c3c4ccccc4c(-c4ccc(-c5nc6ccccc6c6c7ccccc7c7ccccc7c56)cc4)c4ccccc34)ccc2c1]

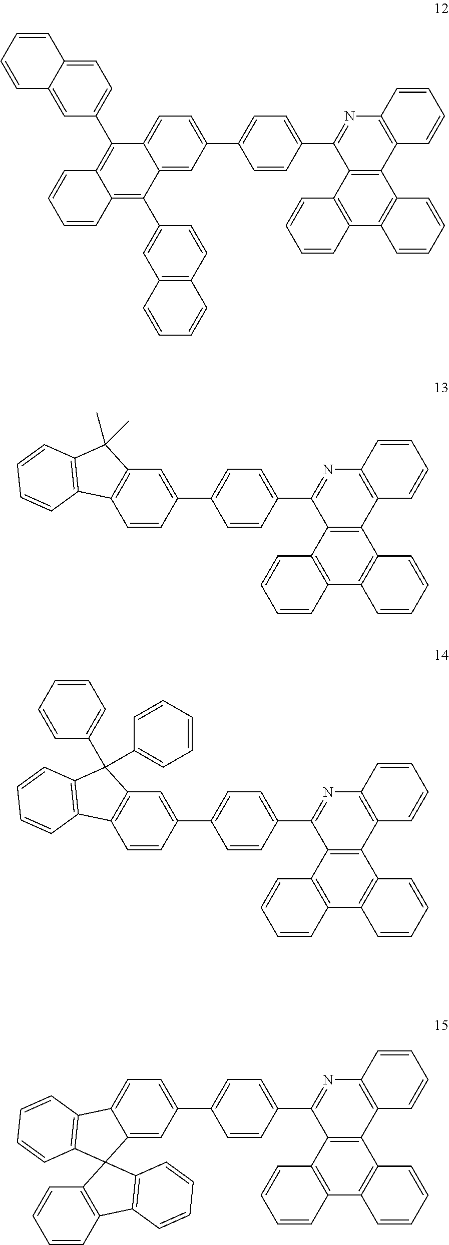 [SMILES: c1ccc2cc(-c3c4ccccc4c(-c4ccc5ccccc5c4)c4cc(-c5ccc(-c6nc7ccccc7c7c8ccccc8c8ccccc8c67)cc5)ccc34)ccc2c1 ; CC1(C)c2ccccc2-c2ccc(-c3ccc(-c4nc5ccccc5c5c6ccccc6c6ccccc6c45)cc3)cc21 ; c1ccc(C2(c3ccccc3)c3ccccc3-c3ccc(-c4ccc(-c5nc6ccccc6c6c7ccccc7c7ccccc7c56)cc4)cc32)cc1 ; c1ccc2c(c1)-c1ccccc1C21c2ccccc2-c2ccc(-c3ccc(-c4nc5ccccc5c5c6ccccc6c6ccccc6c45)cc3)cc21]

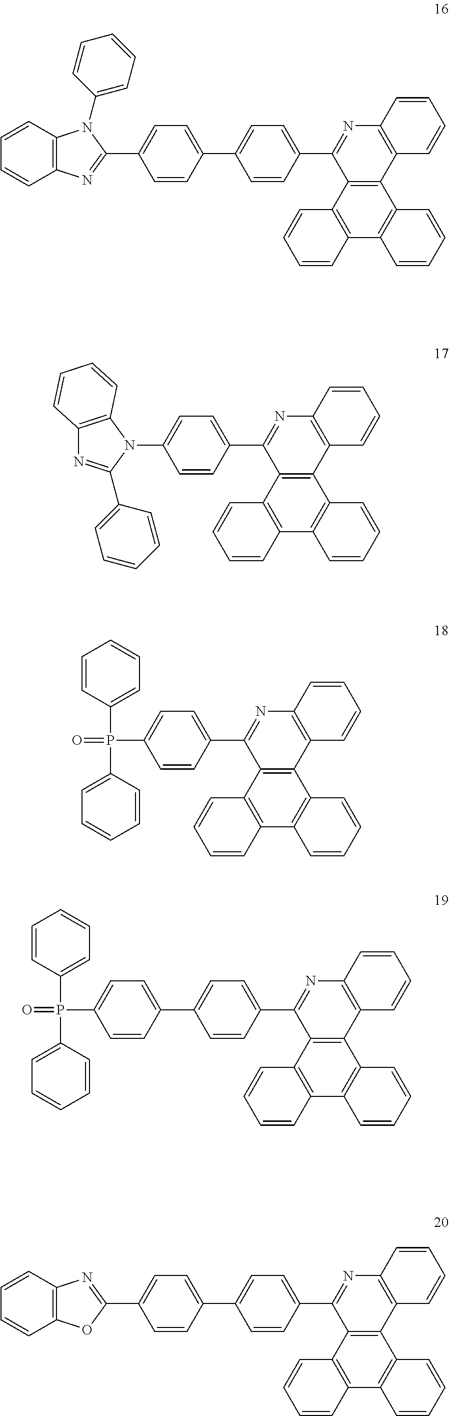 [SMILES: c1ccc(-n2c(-c3ccc(-c4ccc(-c5nc6ccccc6c6c7ccccc7c7ccccc7c56)cc4)cc3)nc3ccccc32)cc1 ; c1ccc(-c2nc3ccccc3n2-c2ccc(-c3nc4ccccc4c4c5ccccc5c5ccccc5c34)cc2)cc1 ; O=P(c1ccccc1)(c1ccccc1)c1ccc(-c2nc3ccccc3c3c4ccccc4c4ccccc4c23)cc1 ; O=P(c1ccccc1)(c1ccccc1)c1ccc(-c2ccc(-c3nc4ccccc4c4c5ccccc5c5ccccc5c34)cc2)cc1 ; c1ccc2oc(-c3ccc(-c4ccc(-c5nc6ccccc6c6c7ccccc7c7ccccc7c56)cc4)cc3)nc2c1]

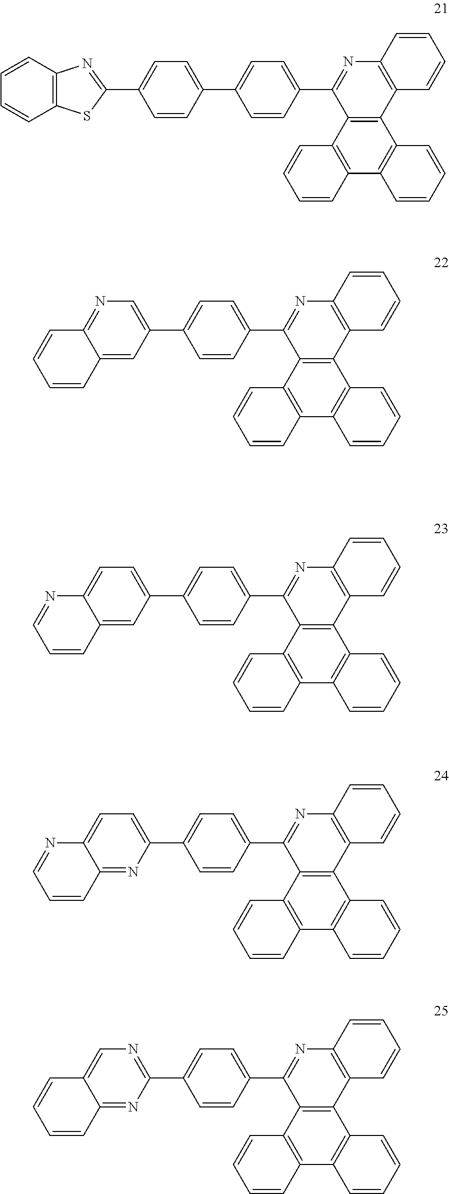 [SMILES: c1ccc2sc(-c3ccc(-c4ccc(-c5nc6ccccc6c6c7ccccc7c7ccccc7c56)cc4)cc3)nc2c1 ; c1ccc2ncc(-c3ccc(-c4nc5ccccc5c5c6ccccc6c6ccccc6c45)cc3)cc2c1 ; c1cnc2ccc(-c3ccc(-c4nc5ccccc5c5c6ccccc6c6ccccc6c45)cc3)cc2c1 ; c1cnc2ccc(-c3ccc(-c4nc5ccccc5c5c6ccccc6c6ccccc6c45)cc3)nc2c1 ; c1ccc2nc(-c3ccc(-c4nc5ccccc5c5c6ccccc6c6ccccc6c45)cc3)ncc2c1]

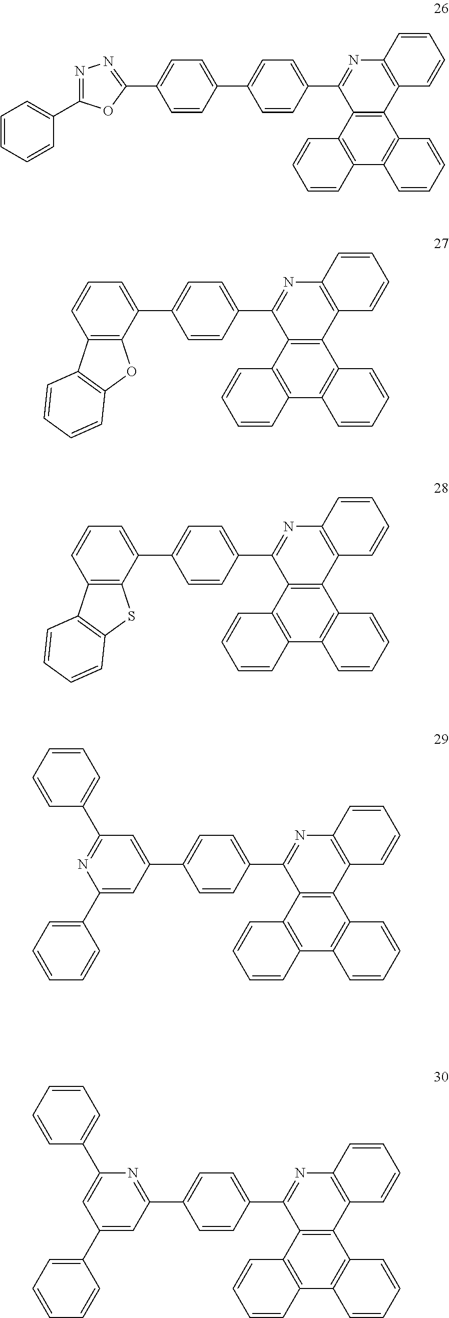 [SMILES: c1ccc(-c2nnc(-c3ccc(-c4ccc(-c5nc6ccccc6c6c7ccccc7c7ccccc7c56)cc4)cc3)o2)cc1 ; c1ccc2c(c1)nc(-c1ccc(-c3cccc4c3oc3ccccc34)cc1)c1c3ccccc3c3ccccc3c21 ; c1ccc2c(c1)nc(-c1ccc(-c3cccc4c3sc3ccccc34)cc1)c1c3ccccc3c3ccccc3c21 ; c1ccc(-c2cc(-c3ccc(-c4nc5ccccc5c5c6ccccc6c6ccccc6c45)cc3)cc(-c3ccccc3)n2)cc1 ; c1ccc(-c2cc(-c3ccccc3)nc(-c3ccc(-c4nc5ccccc5c5c6ccccc6c6ccccc6c45)cc3)c2)cc1]

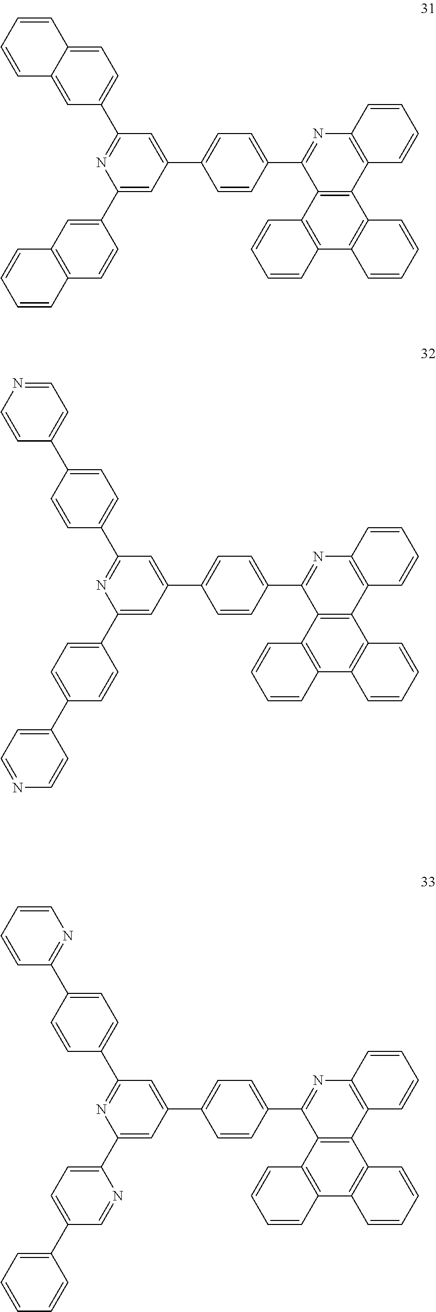 [SMILES: c1ccc2cc(-c3cc(-c4ccc(-c5nc6ccccc6c6c7ccccc7c7ccccc7c56)cc4)cc(-c4ccc5ccccc5c4)n3)ccc2c1 ; c1ccc2c(c1)nc(-c1ccc(-c3cc(-c4ccc(-c5ccncc5)cc4)nc(-c4ccc(-c5ccncc5)cc4)c3)cc1)c1c3ccccc3c3ccccc3c21 ; c1ccc(-c2ccc(-c3cc(-c4ccc(-c5nc6ccccc6c6c7ccccc7c7ccccc7c56)cc4)cc(-c4ccc(-c5ccccn5)cc4)n3)nc2)cc1]

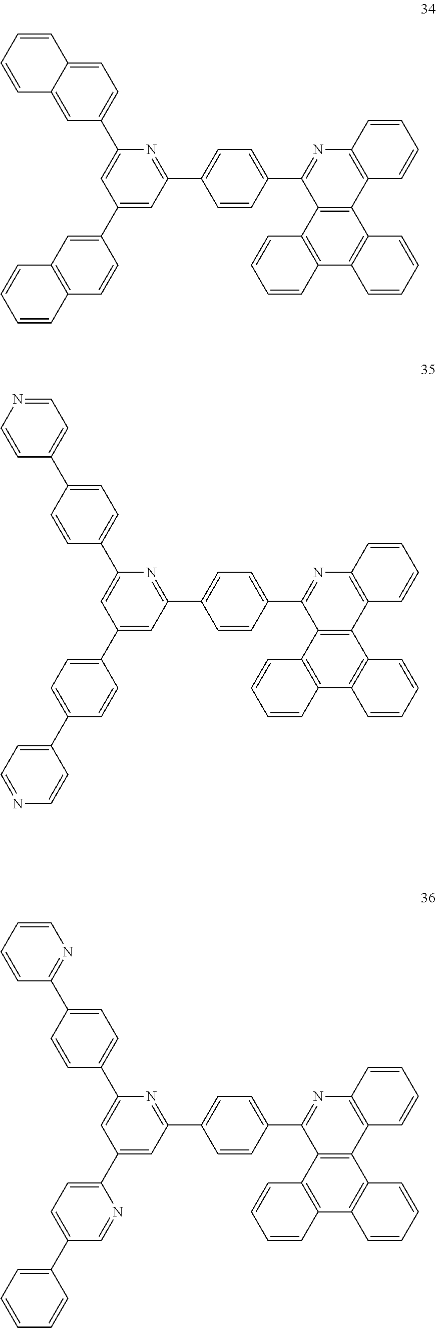 [SMILES: c1ccc2cc(-c3cc(-c4ccc(-c5nc6ccccc6c6c7ccccc7c7ccccc7c56)cc4)nc(-c4ccc5ccccc5c4)c3)ccc2c1 ; c1ccc2c(c1)nc(-c1ccc(-c3cc(-c4ccc(-c5ccncc5)cc4)cc(-c4ccc(-c5ccncc5)cc4)n3)cc1)c1c3ccccc3c3ccccc3c21 ; c1ccc(-c2ccc(-c3cc(-c4ccc(-c5ccccn5)cc4)nc(-c4ccc(-c5nc6ccccc6c6c7ccccc7c7ccccc7c56)cc4)c3)nc2)cc1]

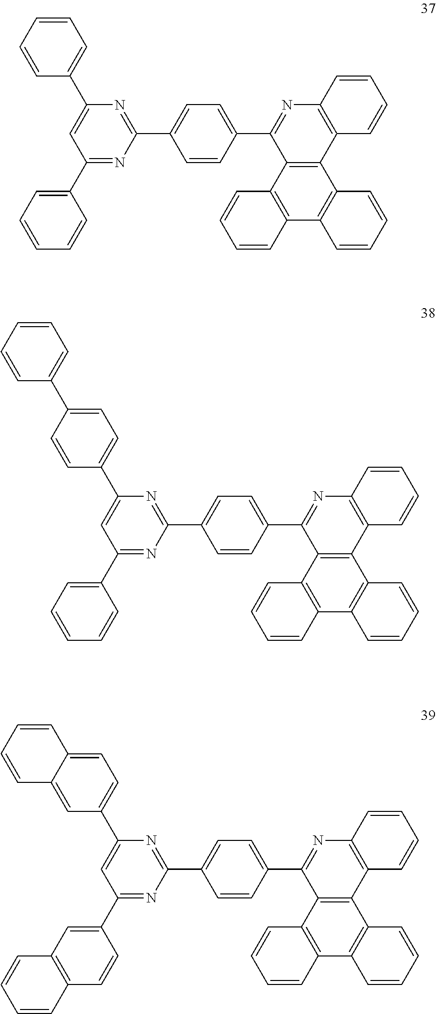 [SMILES: c1ccc(-c2cc(-c3ccccc3)nc(-c3ccc(-c4nc5ccccc5c5c6ccccc6c6ccccc6c45)cc3)n2)cc1 ; c1ccc(-c2ccc(-c3cc(-c4ccccc4)nc(-c4ccc(-c5nc6ccccc6c6c7ccccc7c7ccccc7c56)cc4)n3)cc2)cc1 ; c1ccc2cc(-c3cc(-c4ccc5ccccc5c4)nc(-c4ccc(-c5nc6ccccc6c6c7ccccc7c7ccccc7c56)cc4)n3)ccc2c1]

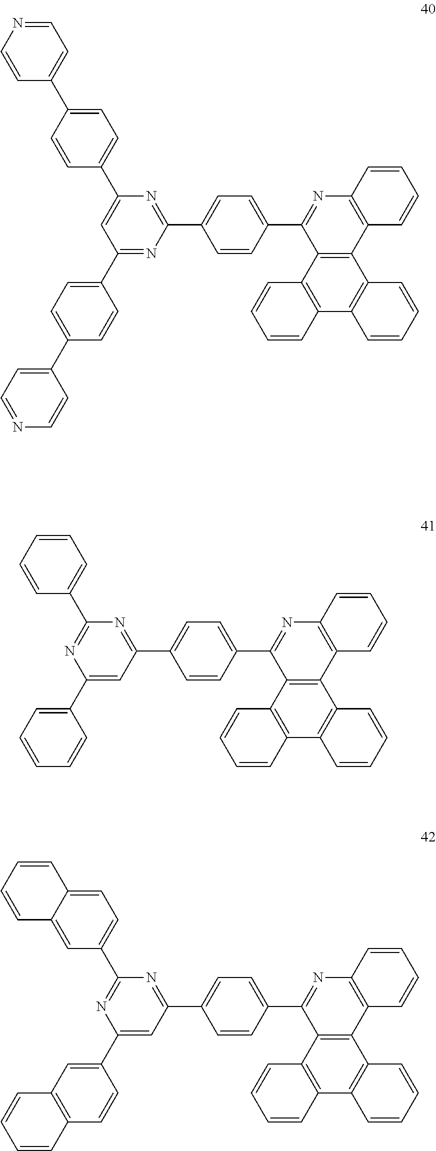 [SMILES: c1ccc2c(c1)nc(-c1ccc(-c3nc(-c4ccc(-c5ccncc5)cc4)cc(-c4ccc(-c5ccncc5)cc4)n3)cc1)c1c3ccccc3c3ccccc3c21 ; c1ccc(-c2cc(-c3ccc(-c4nc5ccccc5c5c6ccccc6c6ccccc6c45)cc3)nc(-c3ccccc3)n2)cc1 ; c1ccc2cc(-c3cc(-c4ccc(-c5nc6ccccc6c6c7ccccc7c7ccccc7c56)cc4)nc(-c4ccc5ccccc5c4)n3)ccc2c1]

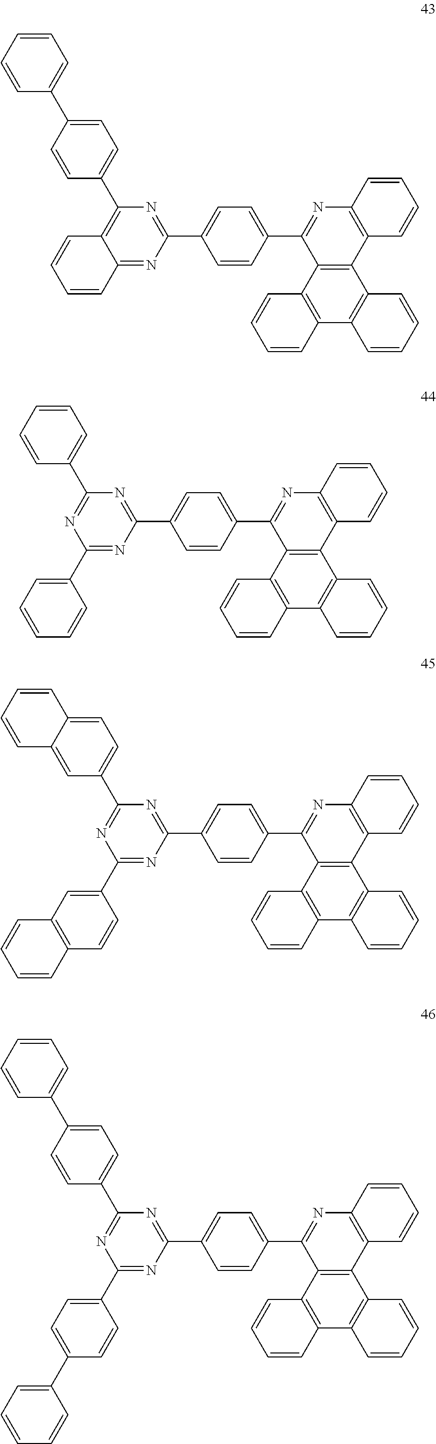 [SMILES: c1ccc(-c2ccc(-c3nc(-c4ccc(-c5nc6ccccc6c6c7ccccc7c7ccccc7c56)cc4)nc4ccccc34)cc2)cc1 ; c1ccc(-c2nc(-c3ccccc3)nc(-c3ccc(-c4nc5ccccc5c5c6ccccc6c6ccccc6c45)cc3)n2)cc1 ; c1ccc2cc(-c3nc(-c4ccc(-c5nc6ccccc6c6c7ccccc7c7ccccc7c56)cc4)nc(-c4ccc5ccccc5c4)n3)ccc2c1 ; c1ccc(-c2ccc(-c3nc(-c4ccc(-c5ccccc5)cc4)nc(-c4ccc(-c5nc6ccccc6c6c7ccccc7c7ccccc7c56)cc4)n3)cc2)cc1]

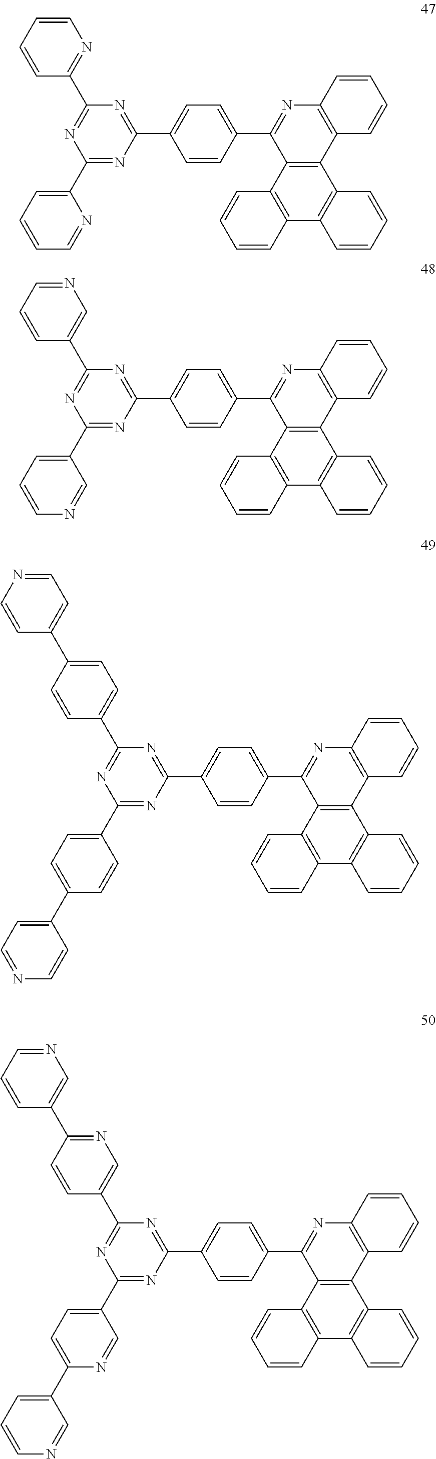

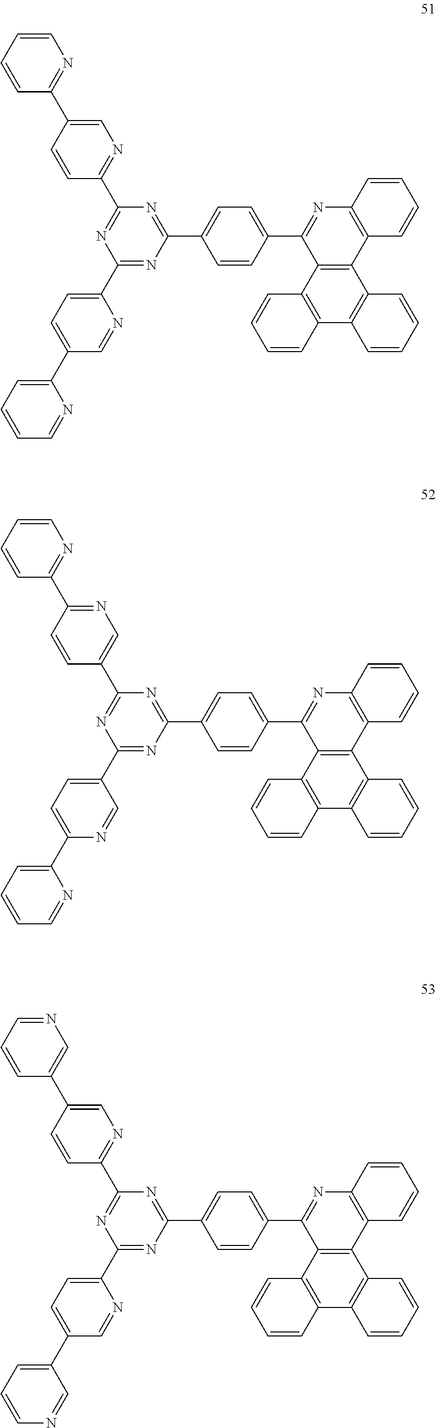 [SMILES: c1ccc(-c2ccc(-c3nc(-c4ccc(-c5nc6ccccc6c6c7ccccc7c7ccccc7c56)cc4)nc(-c4ccc(-c5ccccn5)cn4)n3)nc2)nc1 ; c1ccc(-c2ccc(-c3nc(-c4ccc(-c5nc6ccccc6c6c7ccccc7c7ccccc7c56)cc4)nc(-c4ccc(-c5ccccn5)nc4)n3)cn2)nc1 ; c1cncc(-c2ccc(-c3nc(-c4ccc(-c5nc6ccccc6c6c7ccccc7c7ccccc7c56)cc4)nc(-c4ccc(-c5cccnc5)cn4)n3)nc2)c1]

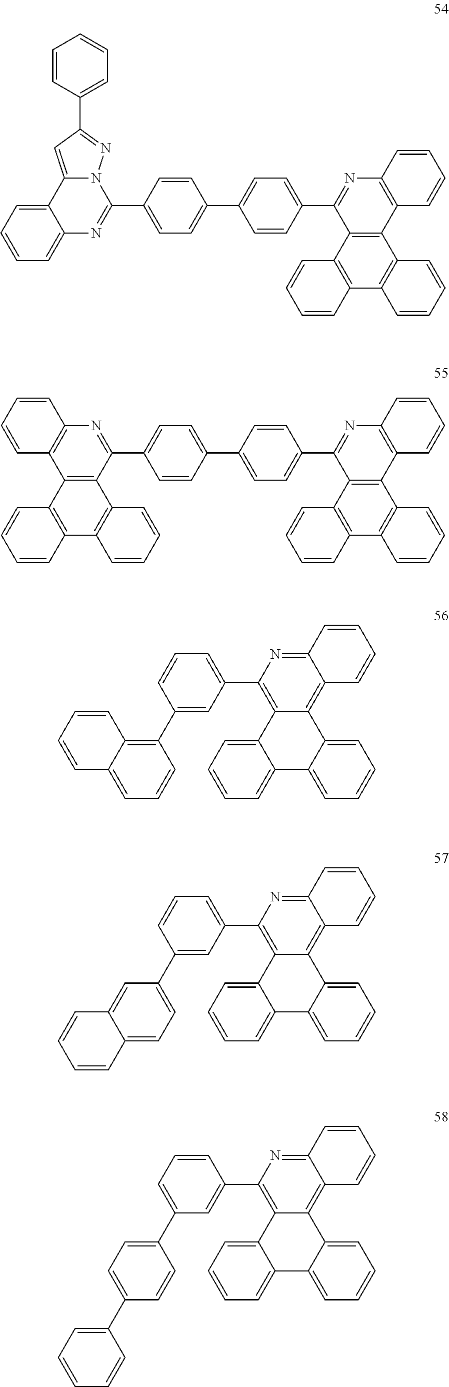 [SMILES: c1ccc(-c2cc3c4ccccc4nc(-c4ccc(-c5ccc(-c6nc7ccccc7c7c8ccccc8c8ccccc8c67)cc5)cc4)n3n2)cc1 ; c1ccc2c(c1)nc(-c1ccc(-c3ccc(-c4nc5ccccc5c5c6ccccc6c6ccccc6c45)cc3)cc1)c1c3ccccc3c3ccccc3c21 ; c1cc(-c2cccc3ccccc23)cc(-c2nc3ccccc3c3c4ccccc4c4ccccc4c23)c1 ; c1cc(-c2ccc3ccccc3c2)cc(-c2nc3ccccc3c3c4ccccc4c4ccccc4c23)c1 ; c1ccc(-c2ccc(-c3cccc(-c4nc5ccccc5c5c6ccccc6c6ccccc6c45)c3)cc2)cc1]

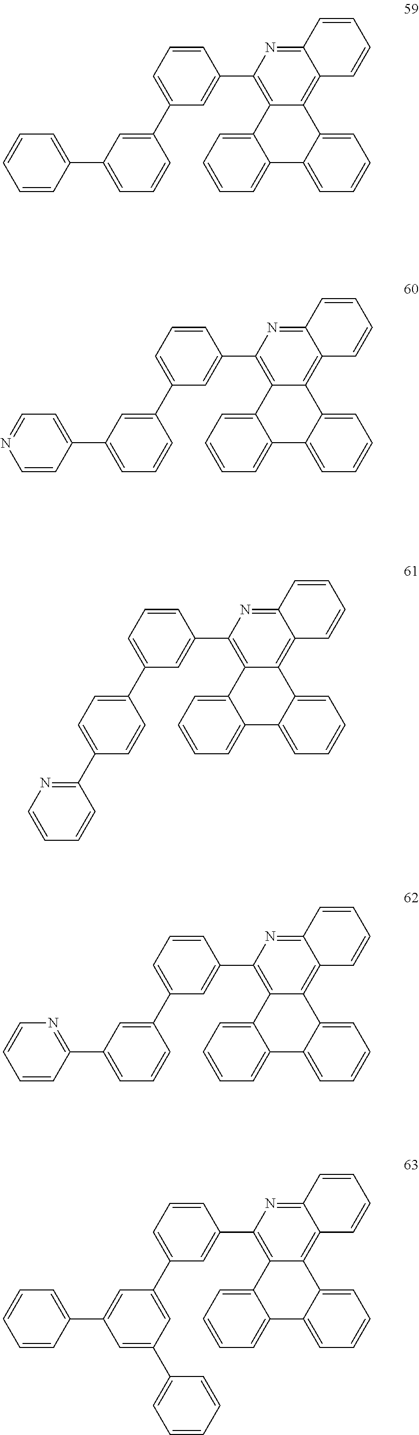 [SMILES: c1ccc(-c2cccc(-c3cccc(-c4nc5ccccc5c5c6ccccc6c6ccccc6c45)c3)c2)cc1 ; c1cc(-c2ccncc2)cc(-c2cccc(-c3nc4ccccc4c4c5ccccc5c5ccccc5c34)c2)c1 ; c1ccc(-c2ccc(-c3cccc(-c4nc5ccccc5c5c6ccccc6c6ccccc6c45)c3)cc2)nc1 ; c1ccc(-c2cccc(-c3cccc(-c4nc5ccccc5c5c6ccccc6c6ccccc6c45)c3)c2)nc1 ; c1ccc(-c2cc(-c3ccccc3)cc(-c3cccc(-c4nc5ccccc5c5c6ccccc6c6ccccc6c45)c3)c2)cc1]

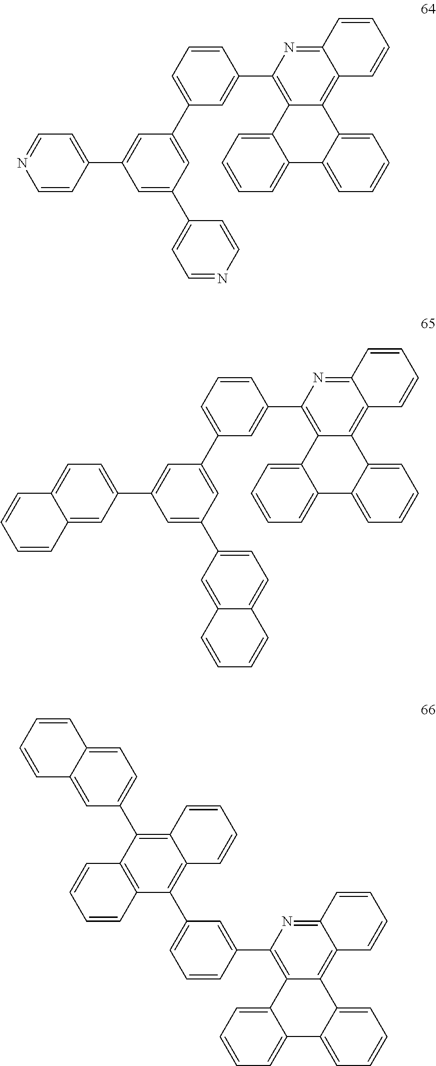 [SMILES: c1cc(-c2cc(-c3ccncc3)cc(-c3ccncc3)c2)cc(-c2nc3ccccc3c3c4ccccc4c4ccccc4c23)c1 ; c1cc(-c2cc(-c3ccc4ccccc4c3)cc(-c3ccc4ccccc4c3)c2)cc(-c2nc3ccccc3c3c4ccccc4c4ccccc4c23)c1 ; c1cc(-c2c3ccccc3c(-c3ccc4ccccc4c3)c3ccccc23)cc(-c2nc3ccccc3c3c4ccccc4c4ccccc4c23)c1]

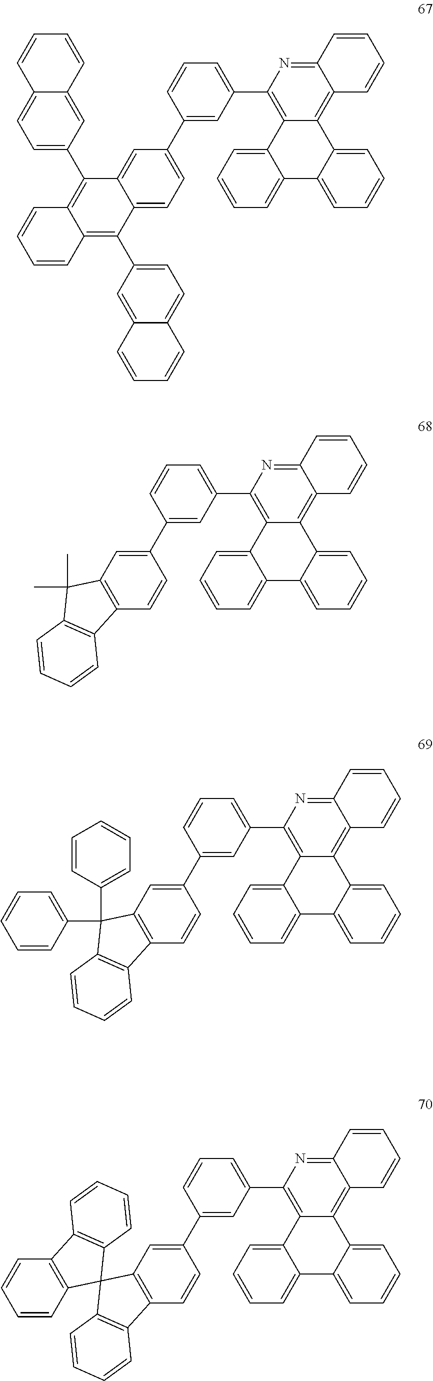 [SMILES: c1cc(-c2ccc3c(-c4ccc5ccccc5c4)c4ccccc4c(-c4ccc5ccccc5c4)c3c2)cc(-c2nc3ccccc3c3c4ccccc4c4ccccc4c23)c1 ; CC1(C)c2ccccc2-c2ccc(-c3cccc(-c4nc5ccccc5c5c6ccccc6c6ccccc6c45)c3)cc21 ; c1ccc(C2(c3ccccc3)c3ccccc3-c3ccc(-c4cccc(-c5nc6ccccc6c6c7ccccc7c7ccccc7c56)c4)cc32)cc1 ; c1cc(-c2ccc3c(c2)C2(c4ccccc4-c4ccccc42)c2ccccc2-3)cc(-c2nc3ccccc3c3c4ccccc4c4ccccc4c23)c1]

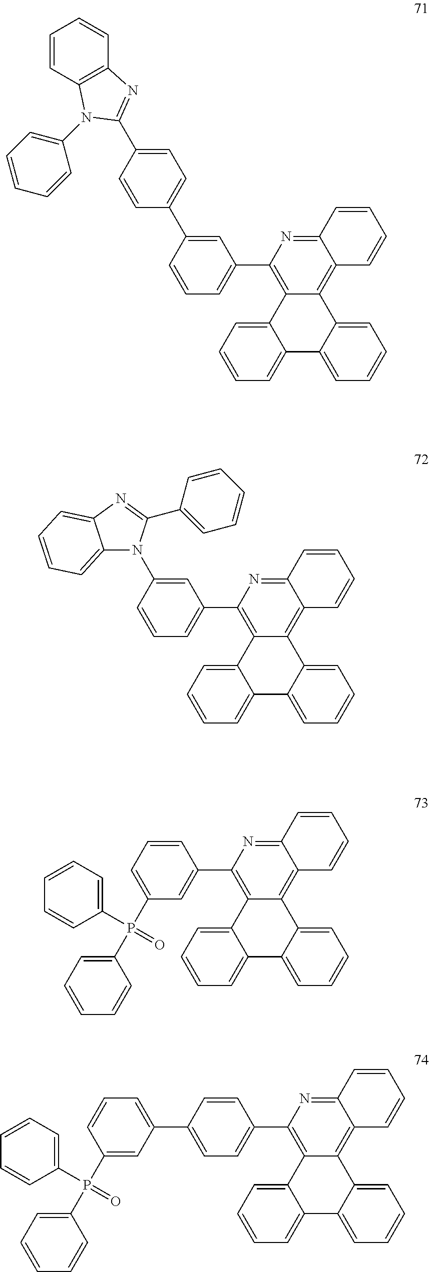 [SMILES: c1ccc(-n2c(-c3ccc(-c4cccc(-c5nc6ccccc6c6c7ccccc7c7ccccc7c56)c4)cc3)nc3ccccc32)cc1 ; c1ccc(-c2nc3ccccc3n2-c2cccc(-c3nc4ccccc4c4c5ccccc5c5ccccc5c34)c2)cc1 ; O=P(c1ccccc1)(c1ccccc1)c1cccc(-c2nc3ccccc3c3c4ccccc4c4ccccc4c23)c1 ; O=P(c1ccccc1)(c1ccccc1)c1cccc(-c2ccc(-c3nc4ccccc4c4c5ccccc5c5ccccc5c34)cc2)c1]

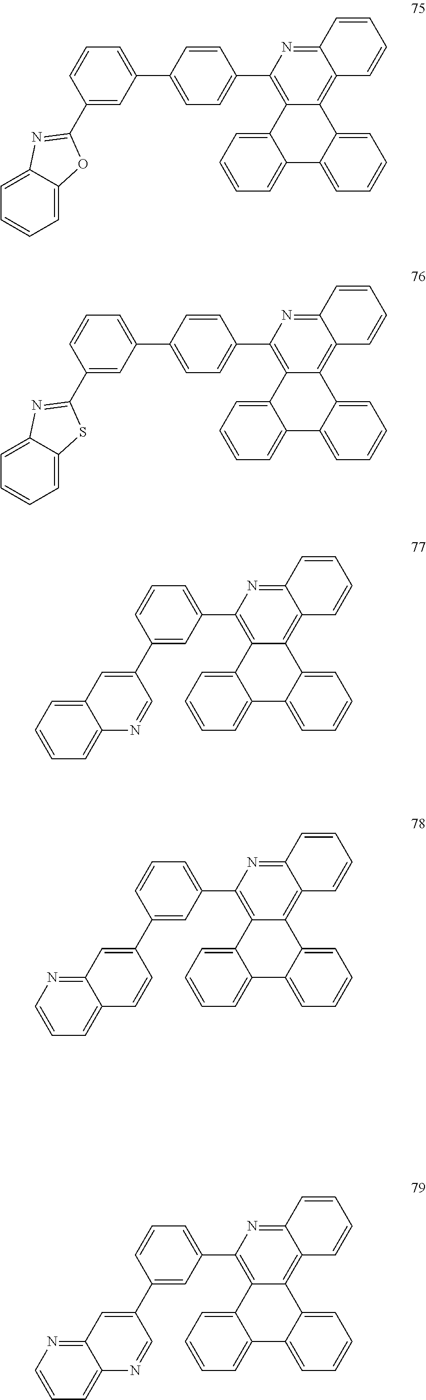 [SMILES: c1cc(-c2ccc(-c3nc4ccccc4c4c5ccccc5c5ccccc5c34)cc2)cc(-c2nc3ccccc3o2)c1 ; c1cc(-c2ccc(-c3nc4ccccc4c4c5ccccc5c5ccccc5c34)cc2)cc(-c2nc3ccccc3s2)c1 ; c1cc(-c2cnc3ccccc3c2)cc(-c2nc3ccccc3c3c4ccccc4c4ccccc4c23)c1 ; c1cc(-c2ccc3cccnc3c2)cc(-c2nc3ccccc3c3c4ccccc4c4ccccc4c23)c1 ; c1cc(-c2cnc3cccnc3c2)cc(-c2nc3ccccc3c3c4ccccc4c4ccccc4c23)c1]

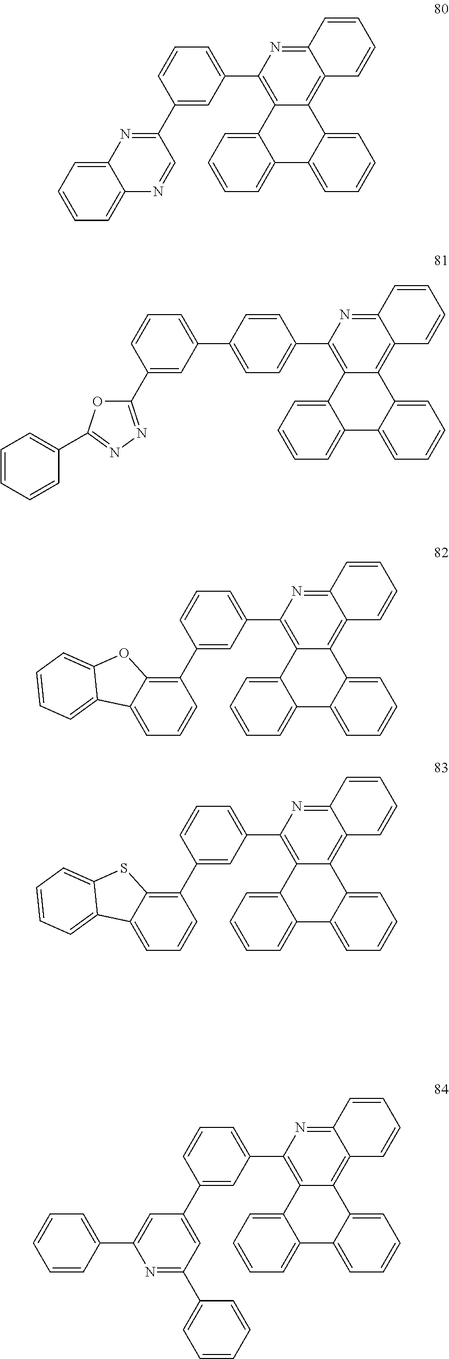

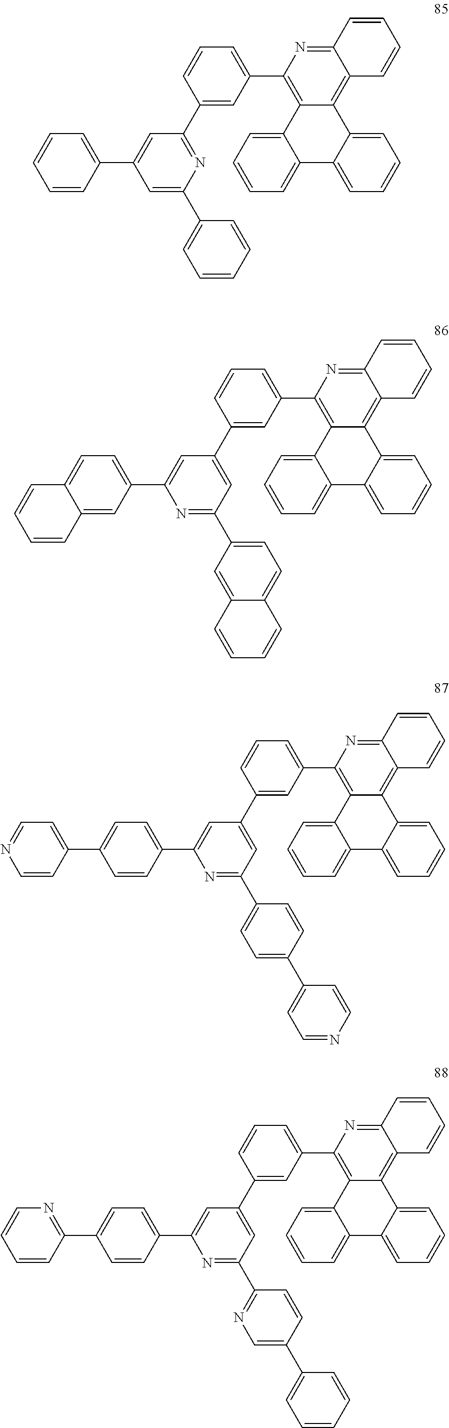 [SMILES: c1ccc(-c2cc(-c3ccccc3)nc(-c3cccc(-c4nc5ccccc5c5c6ccccc6c6ccccc6c45)c3)c2)cc1 ; c1cc(-c2cc(-c3ccc4ccccc4c3)nc(-c3ccc4ccccc4c3)c2)cc(-c2nc3ccccc3c3c4ccccc4c4ccccc4c23)c1 ; c1cc(-c2cc(-c3ccc(-c4ccncc4)cc3)nc(-c3ccc(-c4ccncc4)cc3)c2)cc(-c2nc3ccccc3c3c4ccccc4c4ccccc4c23)c1 ; c1ccc(-c2ccc(-c3cc(-c4cccc(-c5nc6ccccc6c6c7ccccc7c7ccccc7c56)c4)cc(-c4ccc(-c5ccccn5)cc4)n3)nc2)cc1]

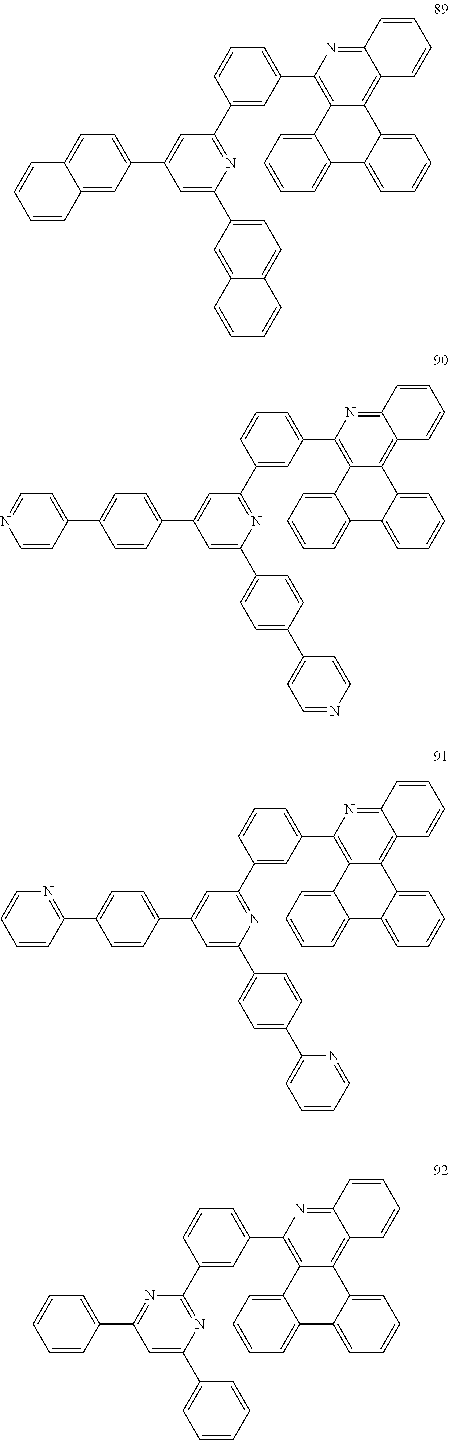 [SMILES: c1cc(-c2cc(-c3ccc4ccccc4c3)cc(-c3ccc4ccccc4c3)n2)cc(-c2nc3ccccc3c3c4ccccc4c4ccccc4c23)c1 ; c1cc(-c2cc(-c3ccc(-c4ccncc4)cc3)cc(-c3ccc(-c4ccncc4)cc3)n2)cc(-c2nc3ccccc3c3c4ccccc4c4ccccc4c23)c1 ; c1ccc(-c2ccc(-c3cc(-c4ccc(-c5ccccn5)cc4)nc(-c4cccc(-c5nc6ccccc6c6c7ccccc7c7ccccc7c56)c4)c3)cc2)nc1 ; c1ccc(-c2cc(-c3ccccc3)nc(-c3cccc(-c4nc5ccccc5c5c6ccccc6c6ccccc6c45)c3)n2)cc1]

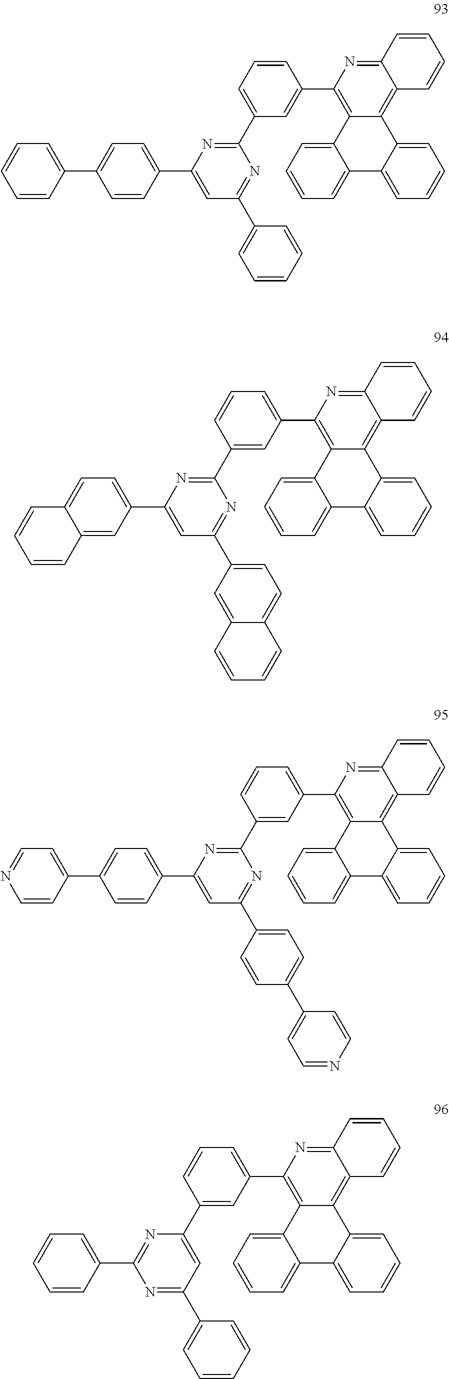 [SMILES: c1ccc(-c2ccc(-c3cc(-c4ccccc4)nc(-c4cccc(-c5nc6ccccc6c6c7ccccc7c7ccccc7c56)c4)n3)cc2)cc1 ; c1cc(-c2nc(-c3ccc4ccccc4c3)cc(-c3ccc4ccccc4c3)n2)cc(-c2nc3ccccc3c3c4ccccc4c4ccccc4c23)c1 ; c1cc(-c2nc(-c3ccc(-c4ccncc4)cc3)cc(-c3ccc(-c4ccncc4)cc3)n2)cc(-c2nc3ccccc3c3c4ccccc4c4ccccc4c23)c1 ; c1ccc(-c2cc(-c3cccc(-c4nc5ccccc5c5c6ccccc6c6ccccc6c45)c3)nc(-c3ccccc3)n2)cc1]

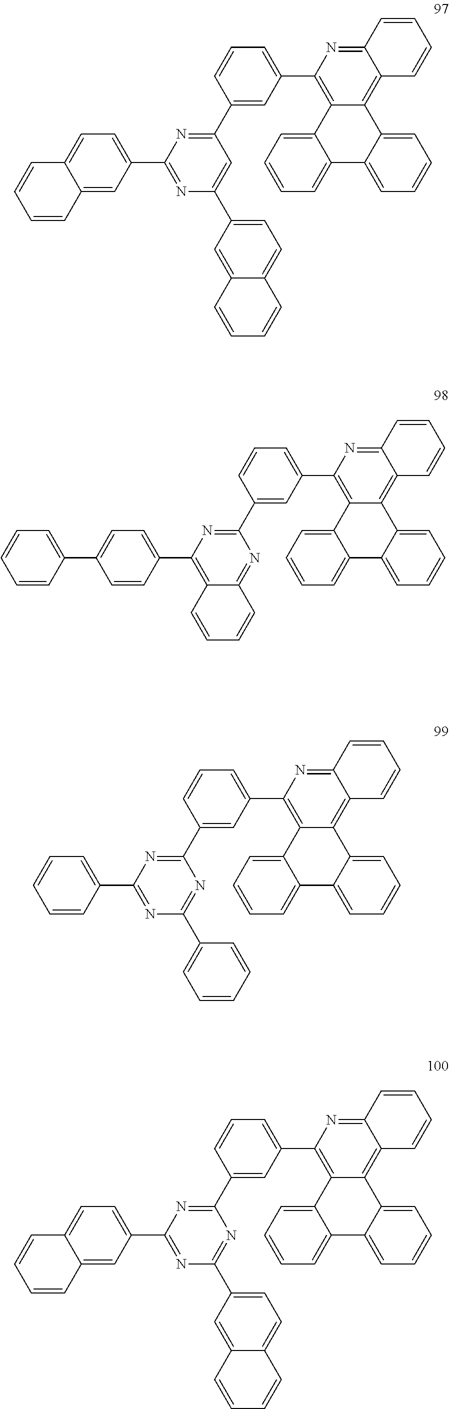 [SMILES: c1cc(-c2cc(-c3ccc4ccccc4c3)nc(-c3ccc4ccccc4c3)n2)cc(-c2nc3ccccc3c3c4ccccc4c4ccccc4c23)c1 ; c1ccc(-c2ccc(-c3nc(-c4cccc(-c5nc6ccccc6c6c7ccccc7c7ccccc7c56)c4)nc4ccccc34)cc2)cc1 ; c1ccc(-c2nc(-c3ccccc3)nc(-c3cccc(-c4nc5ccccc5c5c6ccccc6c6ccccc6c45)c3)n2)cc1 ; c1cc(-c2nc(-c3ccc4ccccc4c3)nc(-c3ccc4ccccc4c3)n2)cc(-c2nc3ccccc3c3c4ccccc4c4ccccc4c23)c1]

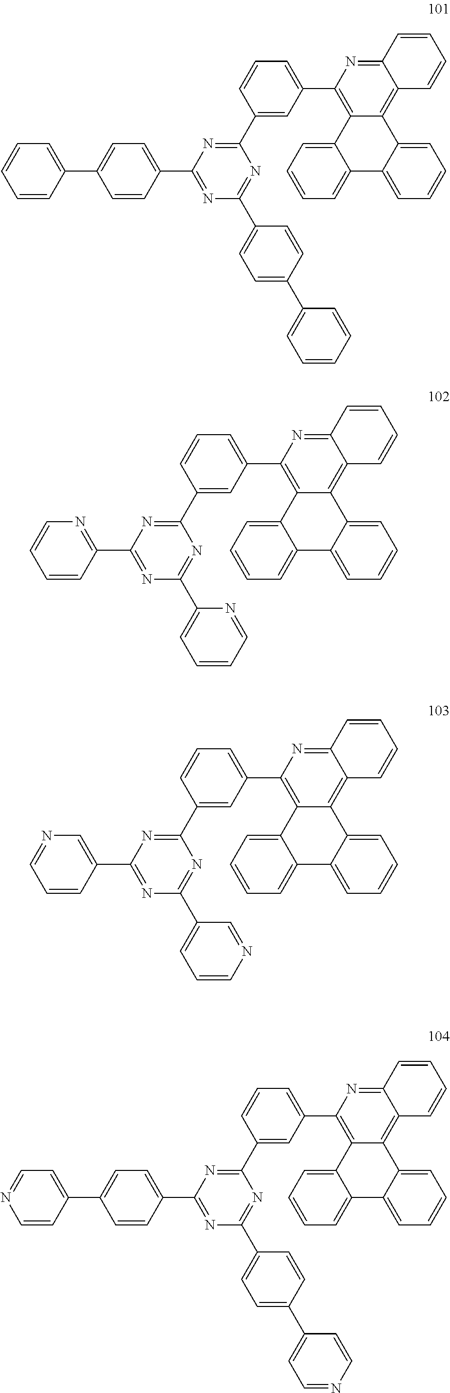 [SMILES: c1ccc(-c2ccc(-c3nc(-c4ccc(-c5ccccc5)cc4)nc(-c4cccc(-c5nc6ccccc6c6c7ccccc7c7ccccc7c56)c4)n3)cc2)cc1 ; c1ccc(-c2nc(-c3cccc(-c4nc5ccccc5c5c6ccccc6c6ccccc6c45)c3)nc(-c3ccccn3)n2)nc1 ; c1cncc(-c2nc(-c3cccnc3)nc(-c3cccc(-c4nc5ccccc5c5c6ccccc6c6ccccc6c45)c3)n2)c1 ; c1cc(-c2nc(-c3ccc(-c4ccncc4)cc3)nc(-c3ccc(-c4ccncc4)cc3)n2)cc(-c2nc3ccccc3c3c4ccccc4c4ccccc4c23)c1]

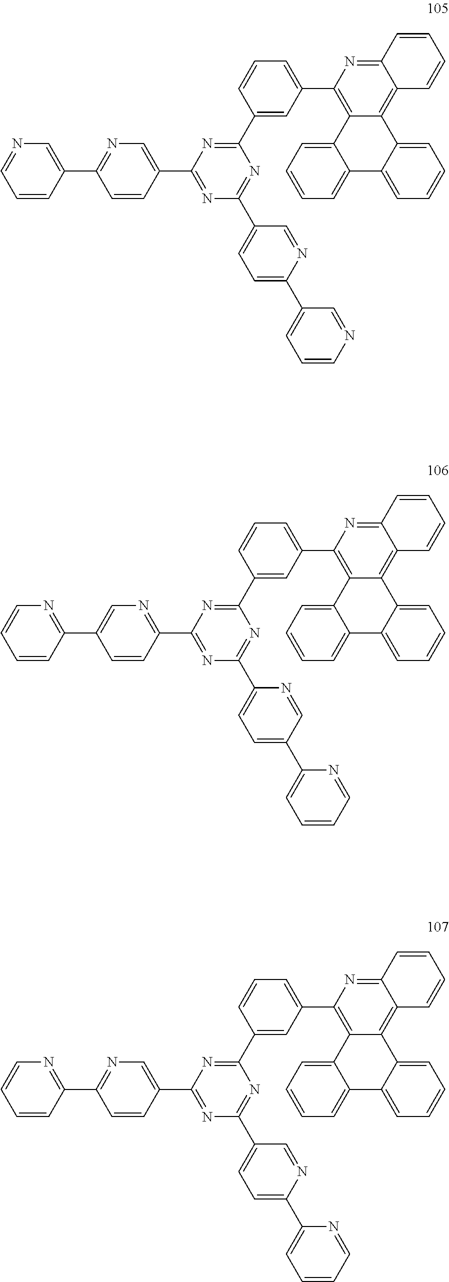 [SMILES: c1cncc(-c2ccc(-c3nc(-c4ccc(-c5cccnc5)nc4)nc(-c4cccc(-c5nc6ccccc6c6c7ccccc7c7ccccc7c56)c4)n3)cn2)c1 ; c1ccc(-c2ccc(-c3nc(-c4cccc(-c5nc6ccccc6c6c7ccccc7c7ccccc7c56)c4)nc(-c4ccc(-c5ccccn5)cn4)n3)nc2)nc1 ; c1ccc(-c2ccc(-c3nc(-c4ccc(-c5ccccn5)nc4)nc(-c4cccc(-c5nc6ccccc6c6c7ccccc7c7ccccc7c56)c4)n3)cn2)nc1]

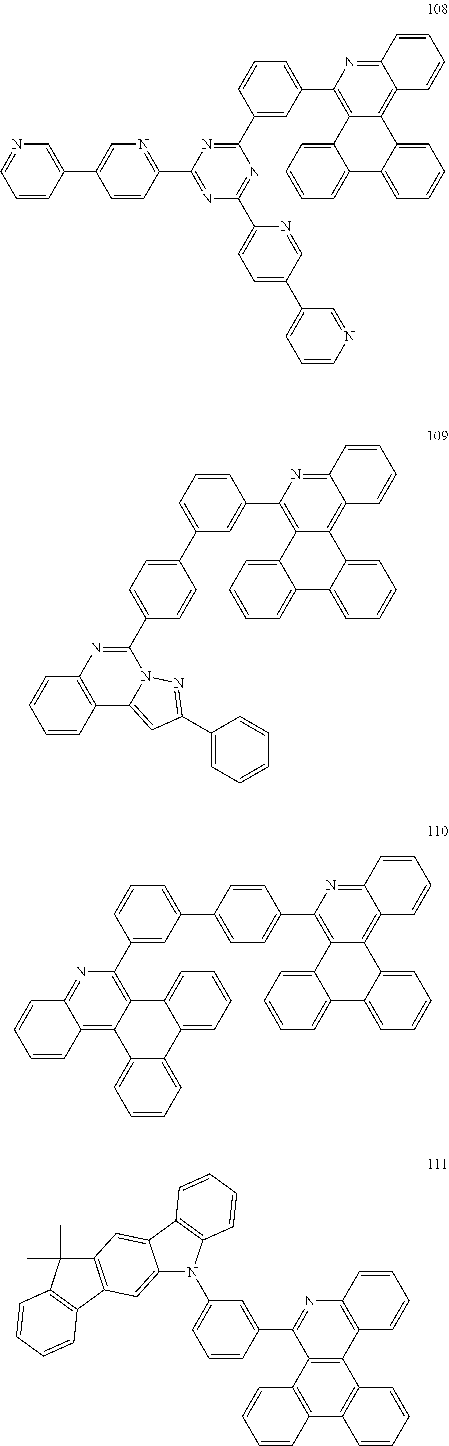 [SMILES: c1cncc(-c2ccc(-c3nc(-c4cccc(-c5nc6ccccc6c6c7ccccc7c7ccccc7c56)c4)nc(-c4ccc(-c5cccnc5)cn4)n3)nc2)c1 ; c1ccc(-c2cc3c4ccccc4nc(-c4ccc(-c5cccc(-c6nc7ccccc7c7c8ccccc8c8ccccc8c67)c5)cc4)n3n2)cc1 ; c1cc(-c2ccc(-c3nc4ccccc4c4c5ccccc5c5ccccc5c34)cc2)cc(-c2nc3ccccc3c3c4ccccc4c4ccccc4c23)c1 ; CC1(C)c2ccccc2-c2cc3c(cc21)c1ccccc1n3-c1cccc(-c2nc3ccccc3c3c4ccccc4c4ccccc4c23)c1]

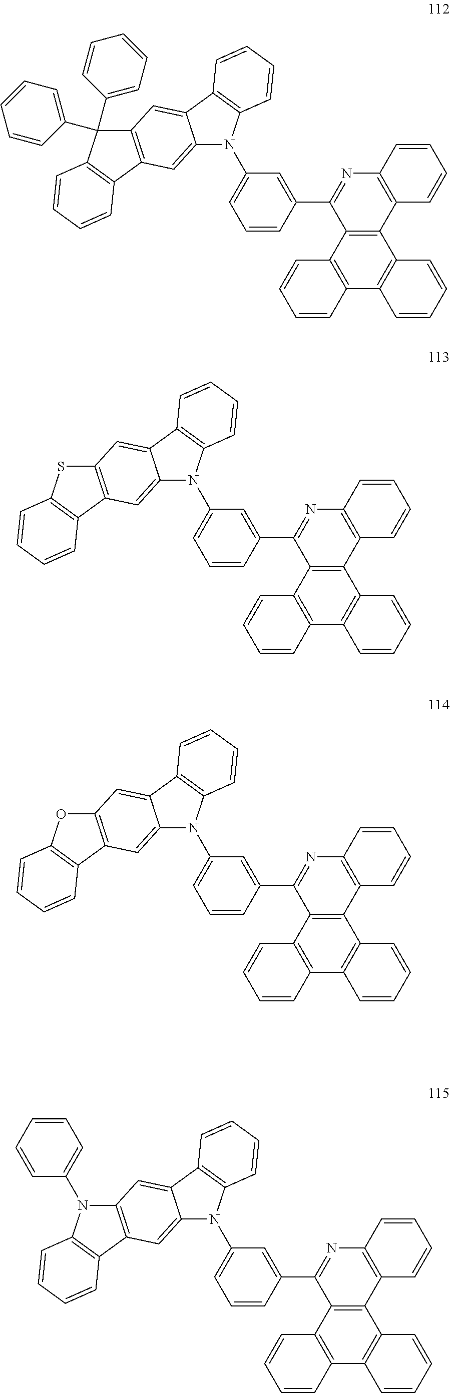 [SMILES: c1ccc(C2(c3ccccc3)c3ccccc3-c3cc4c(cc32)c2ccccc2n4-c2cccc(-c3nc4ccccc4c4c5ccccc5c5ccccc5c34)c2)cc1 ; c1cc(-c2nc3ccccc3c3c4ccccc4c4ccccc4c23)cc(-n2c3ccccc3c3cc4sc5ccccc5c4cc32)c1 ; c1cc(-c2nc3ccccc3c3c4ccccc4c4ccccc4c23)cc(-n2c3ccccc3c3cc4oc5ccccc5c4cc32)c1 ; c1ccc(-n2c3ccccc3c3cc4c(cc32)c2ccccc2n4-c2cccc(-c3nc4ccccc4c4c5ccccc5c5ccccc5c34)c2)cc1]

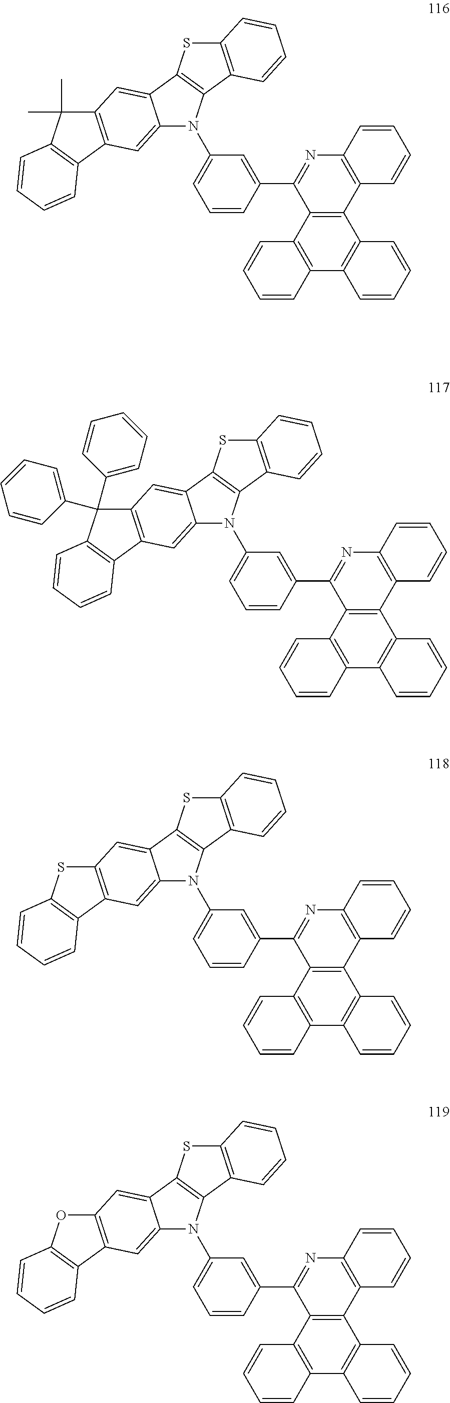 [SMILES: CC1(C)c2ccccc2-c2cc3c(cc21)c1sc2ccccc2c1n3-c1cccc(-c2nc3ccccc3c3c4ccccc4c4ccccc4c23)c1 ; c1ccc(C2(c3ccccc3)c3ccccc3-c3cc4c(cc32)c2sc3ccccc3c2n4-c2cccc(-c3nc4ccccc4c4c5ccccc5c5ccccc5c34)c2)cc1 ; c1cc(-c2nc3ccccc3c3c4ccccc4c4ccccc4c23)cc(-n2c3cc4c(cc3c3sc5ccccc5c32)sc2ccccc24)c1 ; c1cc(-c2nc3ccccc3c3c4ccccc4c4ccccc4c23)cc(-n2c3cc4c(cc3c3sc5ccccc5c32)oc2ccccc24)c1]

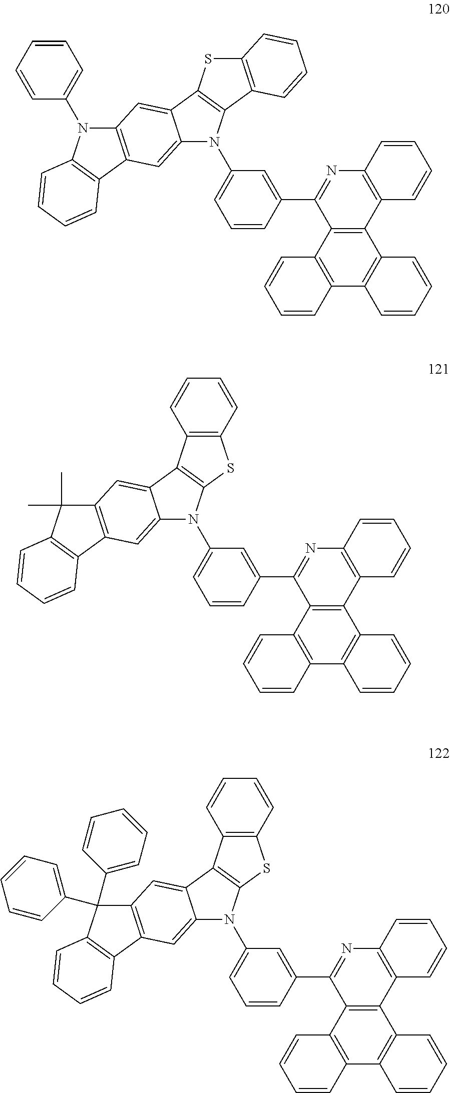 [SMILES: c1ccc(-n2c3ccccc3c3cc4c(cc32)c2sc3ccccc3c2n4-c2cccc(-c3nc4ccccc4c4c5ccccc5c5ccccc5c34)c2)cc1 ; CC1(C)c2ccccc2-c2cc3c(cc21)c1c2ccccc2sc1n3-c1cccc(-c2nc3ccccc3c3c4ccccc4c4ccccc4c23)c1 ; c1ccc(C2(c3ccccc3)c3ccccc3-c3cc4c(cc32)c2c3ccccc3sc2n4-c2cccc(-c3nc4ccccc4c4c5ccccc5c5ccccc5c34)c2)cc1]

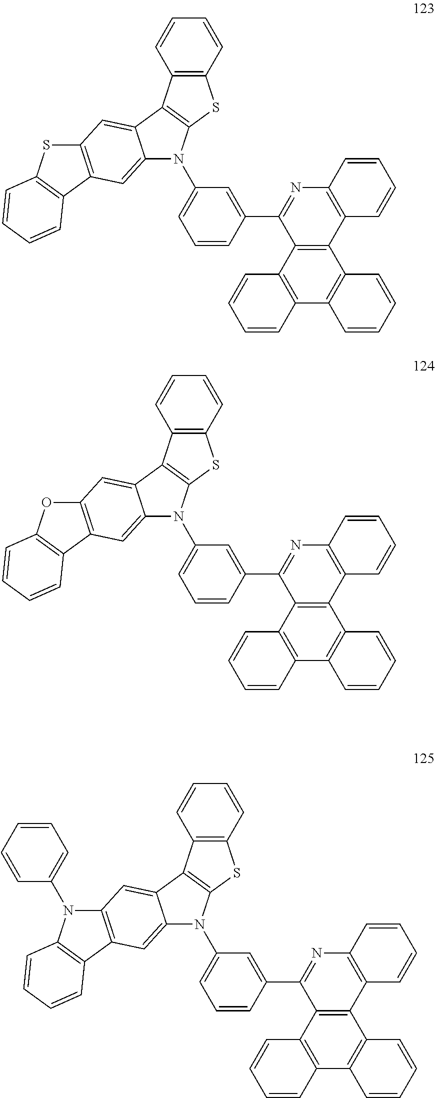 [SMILES: c1cc(-c2nc3ccccc3c3c4ccccc4c4ccccc4c23)cc(-n2c3cc4c(cc3c3c5ccccc5sc32)sc2ccccc24)c1 ; c1cc(-c2nc3ccccc3c3c4ccccc4c4ccccc4c23)cc(-n2c3cc4c(cc3c3c5ccccc5sc32)oc2ccccc24)c1 ; c1ccc(-n2c3ccccc3c3cc4c(cc32)c2c3ccccc3sc2n4-c2cccc(-c3nc4ccccc4c4c5ccccc5c5ccccc5c34)c2)cc1]

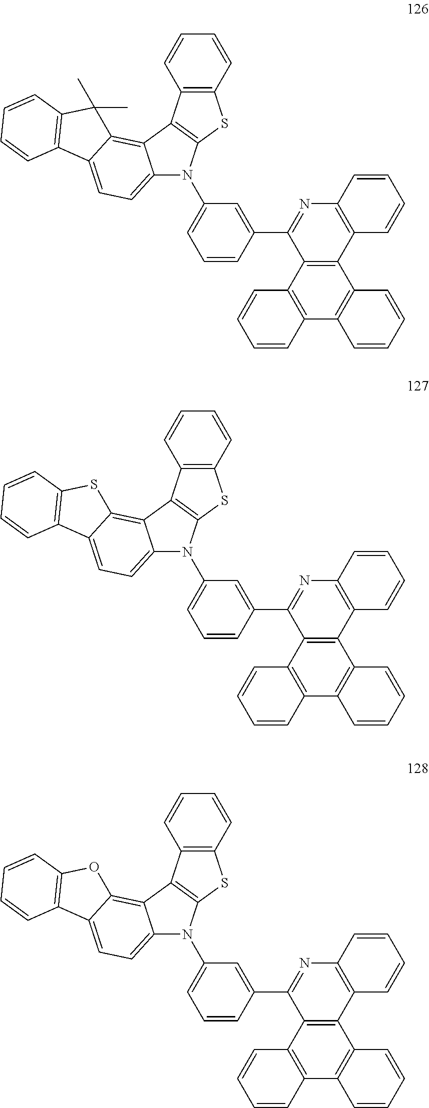 [SMILES: CC1(C)c2ccccc2-c2ccc3c(c21)c1c2ccccc2sc1n3-c1cccc(-c2nc3ccccc3c3c4ccccc4c4ccccc4c23)c1 ; c1cc(-c2nc3ccccc3c3c4ccccc4c4ccccc4c23)cc(-n2c3ccc4c5ccccc5sc4c3c3c4ccccc4sc32)c1 ; c1cc(-c2nc3ccccc3c3c4ccccc4c4ccccc4c23)cc(-n2c3ccc4c5ccccc5oc4c3c3c4ccccc4sc32)c1]

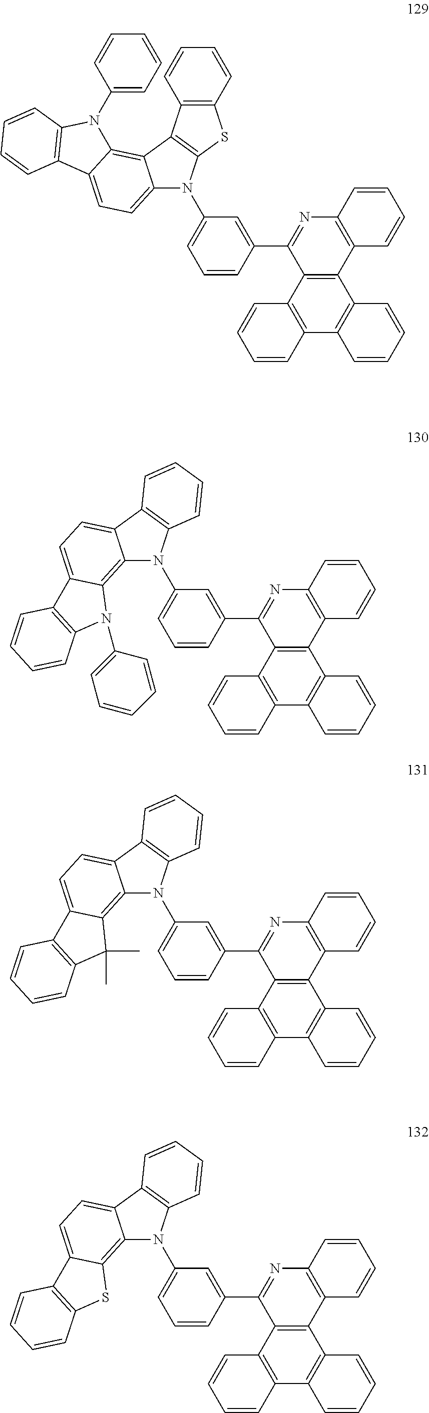 [SMILES: c1ccc(-n2c3ccccc3c3ccc4c(c5c6ccccc6sc5n4-c4cccc(-c5nc6ccccc6c6c7ccccc7c7ccccc7c56)c4)c32)cc1 ; c1ccc(-n2c3ccccc3c3ccc4c5ccccc5n(-c5cccc(-c6nc7ccccc7c7c8ccccc8c8ccccc8c67)c5)c4c32)cc1 ; CC1(C)c2ccccc2-c2ccc3c4ccccc4n(-c4cccc(-c5nc6ccccc6c6c7ccccc7c7ccccc7c56)c4)c3c21 ; c1cc(-c2nc3ccccc3c3c4ccccc4c4ccccc4c23)cc(-n2c3ccccc3c3ccc4c5ccccc5sc4c32)c1]

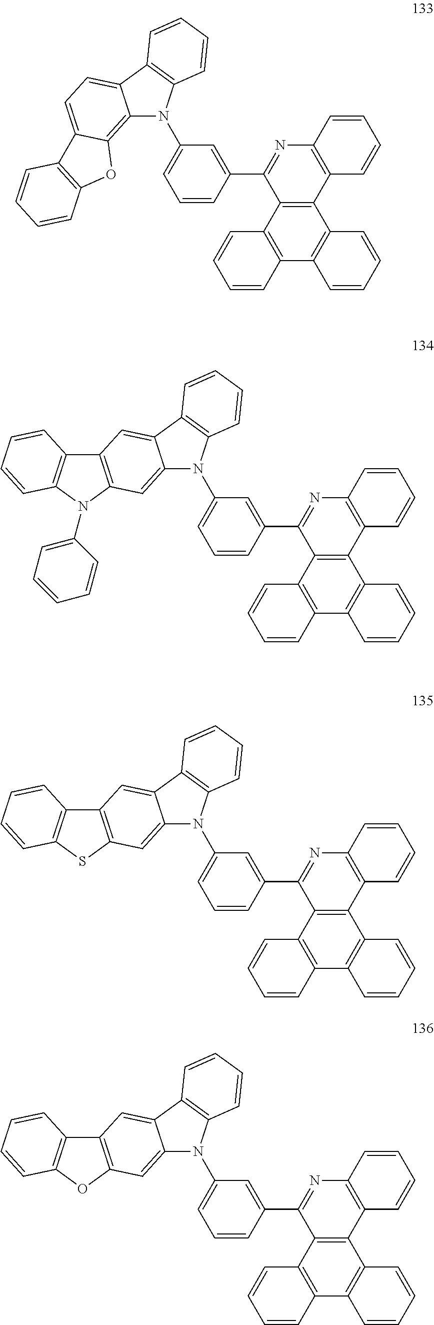 [SMILES: c1cc(-c2nc3ccccc3c3c4ccccc4c4ccccc4c23)cc(-n2c3ccccc3c3ccc4c5ccccc5oc4c32)c1 ; c1ccc(-n2c3ccccc3c3cc4c5ccccc5n(-c5cccc(-c6nc7ccccc7c7c8ccccc8c8ccccc8c67)c5)c4cc32)cc1 ; c1cc(-c2nc3ccccc3c3c4ccccc4c4ccccc4c23)cc(-n2c3ccccc3c3cc4c(cc32)sc2ccccc24)c1 ; c1cc(-c2nc3ccccc3c3c4ccccc4c4ccccc4c23)cc(-n2c3ccccc3c3cc4c(cc32)oc2ccccc24)c1]

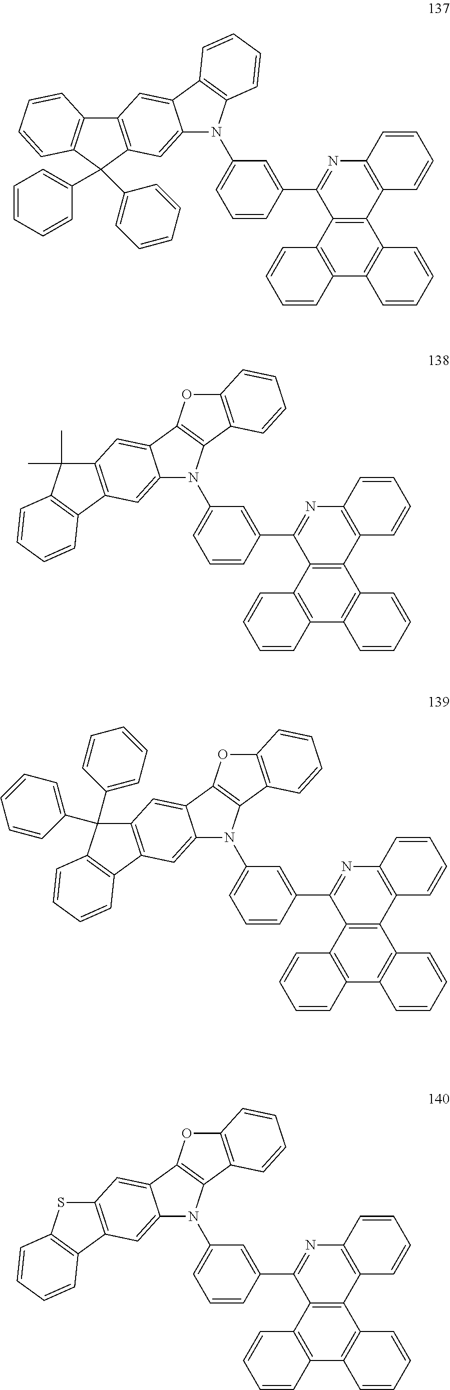 [SMILES: c1ccc(C2(c3ccccc3)c3ccccc3-c3cc4c5ccccc5n(-c5cccc(-c6nc7ccccc7c7c8ccccc8c8ccccc8c67)c5)c4cc32)cc1 ; CC1(C)c2ccccc2-c2cc3c(cc21)c1oc2ccccc2c1n3-c1cccc(-c2nc3ccccc3c3c4ccccc4c4ccccc4c23)c1 ; c1ccc(C2(c3ccccc3)c3ccccc3-c3cc4c(cc32)c2oc3ccccc3c2n4-c2cccc(-c3nc4ccccc4c4c5ccccc5c5ccccc5c34)c2)cc1 ; c1cc(-c2nc3ccccc3c3c4ccccc4c4ccccc4c23)cc(-n2c3cc4c(cc3c3oc5ccccc5c32)sc2ccccc24)c1]

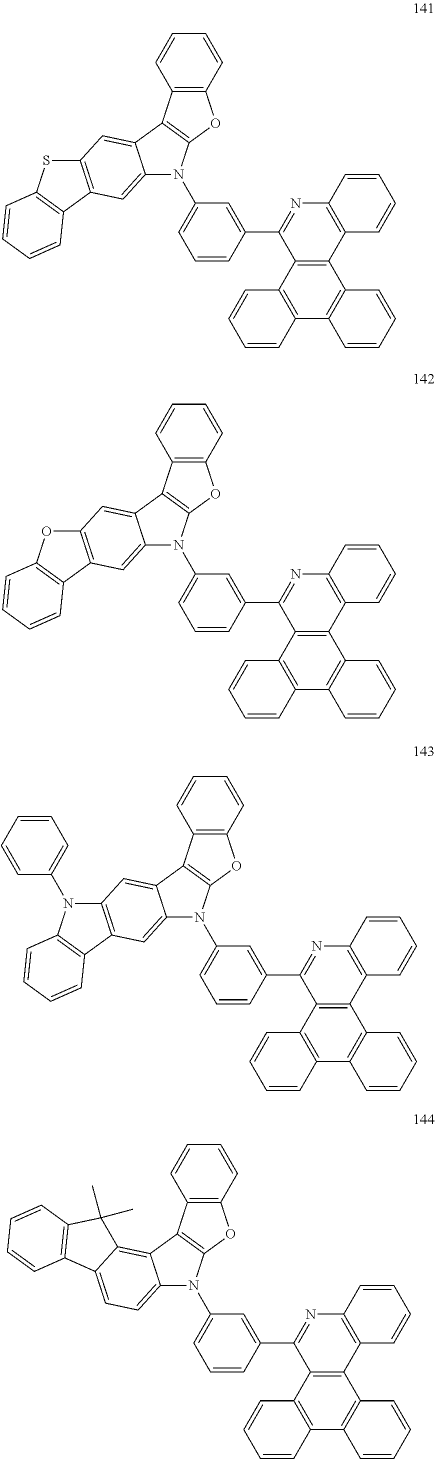 [SMILES: c1cc(-c2nc3ccccc3c3c4ccccc4c4ccccc4c23)cc(-n2c3cc4c(cc3c3c5ccccc5oc32)sc2ccccc24)c1 ; c1cc(-c2nc3ccccc3c3c4ccccc4c4ccccc4c23)cc(-n2c3cc4c(cc3c3c5ccccc5oc32)oc2ccccc24)c1 ; c1ccc(-n2c3ccccc3c3cc4c(cc32)c2c3ccccc3oc2n4-c2cccc(-c3nc4ccccc4c4c5ccccc5c5ccccc5c34)c2)cc1 ; CC1(C)c2ccccc2-c2ccc3c(c21)c1c2ccccc2oc1n3-c1cccc(-c2nc3ccccc3c3c4ccccc4c4ccccc4c23)c1]

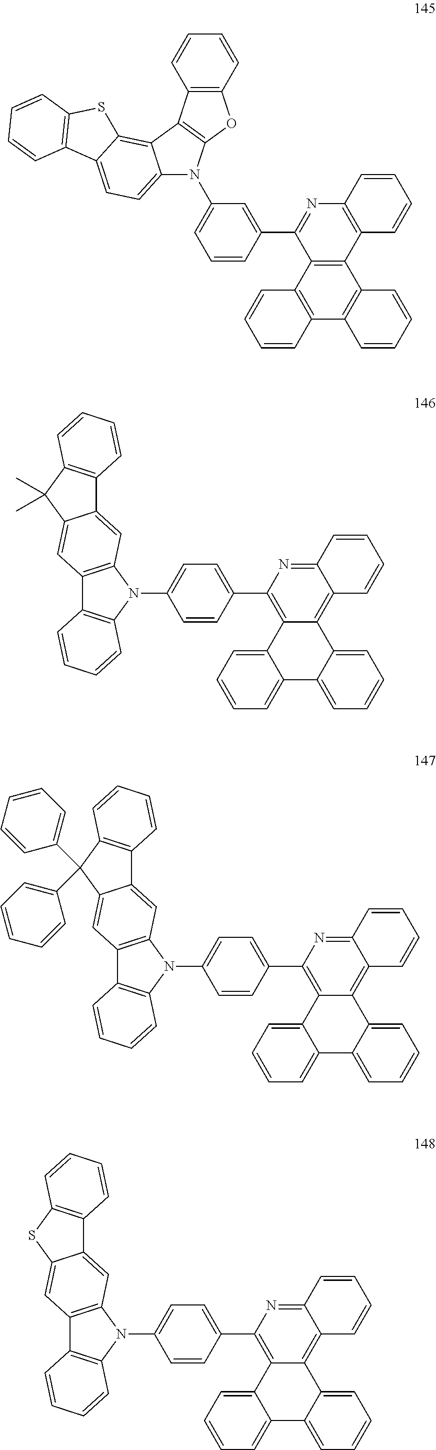 [SMILES: c1cc(-c2nc3ccccc3c3c4ccccc4c4ccccc4c23)cc(-n2c3ccc4c5ccccc5sc4c3c3c4ccccc4oc32)c1 ; CC1(C)c2ccccc2-c2cc3c(cc21)c1ccccc1n3-c1ccc(-c2nc3ccccc3c3c4ccccc4c4ccccc4c23)cc1 ; c1ccc(C2(c3ccccc3)c3ccccc3-c3cc4c(cc32)c2ccccc2n4-c2ccc(-c3nc4ccccc4c4c5ccccc5c5ccccc5c34)cc2)cc1 ; c1ccc2c(c1)nc(-c1ccc(-n3c4ccccc4c4cc5sc6ccccc6c5cc43)cc1)c1c3ccccc3c3ccccc3c21]

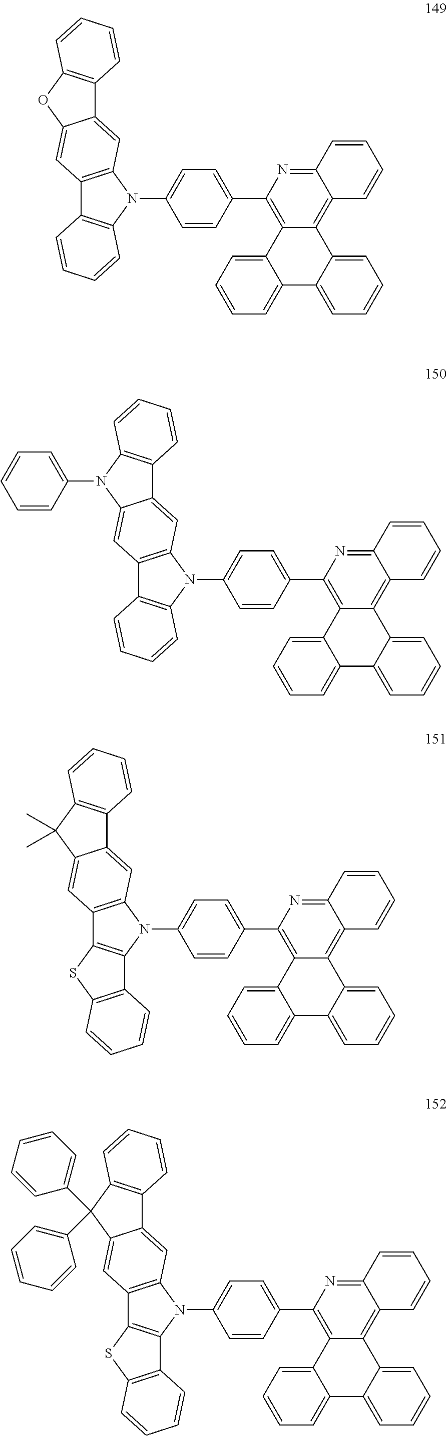 [SMILES: c1ccc2c(c1)nc(-c1ccc(-n3c4ccccc4c4cc5oc6ccccc6c5cc43)cc1)c1c3ccccc3c3ccccc3c21 ; c1ccc(-n2c3ccccc3c3cc4c(cc32)c2ccccc2n4-c2ccc(-c3nc4ccccc4c4c5ccccc5c5ccccc5c34)cc2)cc1 ; CC1(C)c2ccccc2-c2cc3c(cc21)c1sc2ccccc2c1n3-c1ccc(-c2nc3ccccc3c3c4ccccc4c4ccccc4c23)cc1 ; c1ccc(C2(c3ccccc3)c3ccccc3-c3cc4c(cc32)c2sc3ccccc3c2n4-c2ccc(-c3nc4ccccc4c4c5ccccc5c5ccccc5c34)cc2)cc1]

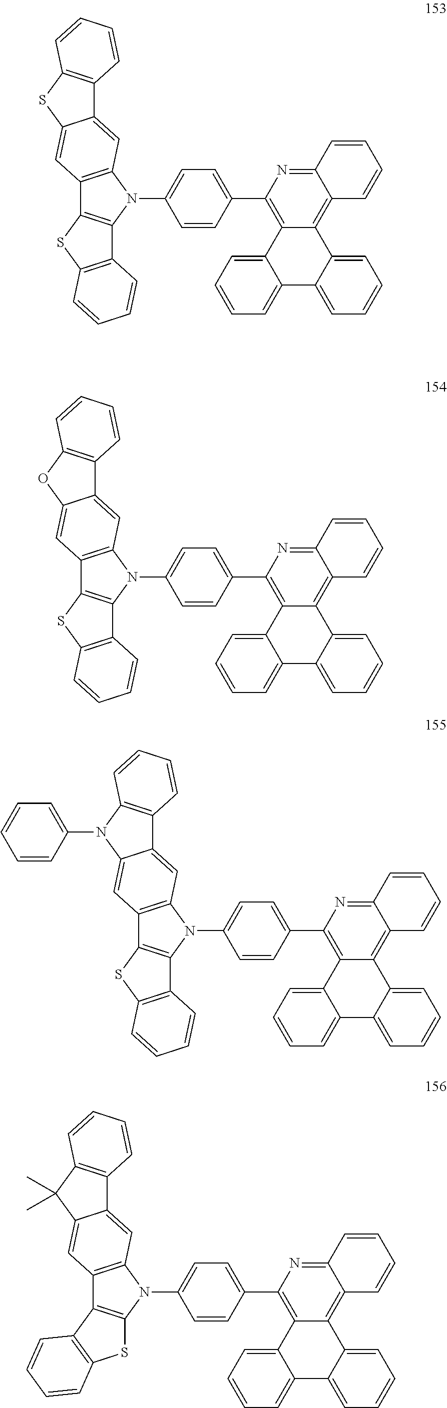 [SMILES: c1ccc2c(c1)nc(-c1ccc(-n3c4cc5c(cc4c4sc6ccccc6c43)sc3ccccc35)cc1)c1c3ccccc3c3ccccc3c21 ; c1ccc2c(c1)nc(-c1ccc(-n3c4cc5c(cc4c4sc6ccccc6c43)oc3ccccc35)cc1)c1c3ccccc3c3ccccc3c21 ; c1ccc(-n2c3ccccc3c3cc4c(cc32)c2sc3ccccc3c2n4-c2ccc(-c3nc4ccccc4c4c5ccccc5c5ccccc5c34)cc2)cc1 ; CC1(C)c2ccccc2-c2cc3c(cc21)c1c2ccccc2sc1n3-c1ccc(-c2nc3ccccc3c3c4ccccc4c4ccccc4c23)cc1]

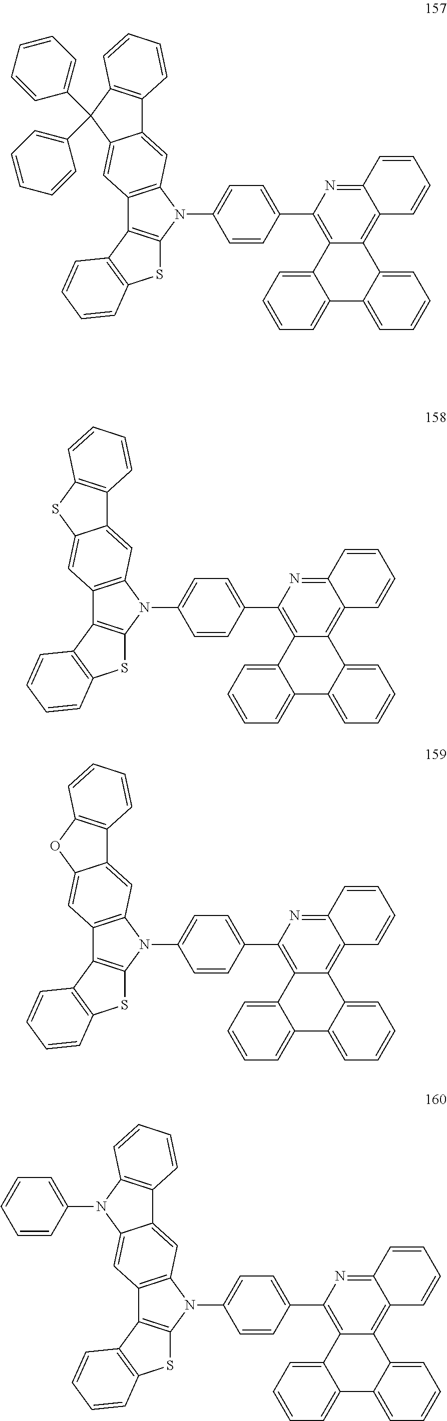 [SMILES: c1ccc(C2(c3ccccc3)c3ccccc3-c3cc4c(cc32)c2c3ccccc3sc2n4-c2ccc(-c3nc4ccccc4c4c5ccccc5c5ccccc5c34)cc2)cc1 ; c1ccc2c(c1)nc(-c1ccc(-n3c4cc5c(cc4c4c6ccccc6sc43)sc3ccccc35)cc1)c1c3ccccc3c3ccccc3c21 ; c1ccc2c(c1)nc(-c1ccc(-n3c4cc5c(cc4c4c6ccccc6sc43)oc3ccccc35)cc1)c1c3ccccc3c3ccccc3c21 ; c1ccc(-n2c3ccccc3c3cc4c(cc32)c2c3ccccc3sc2n4-c2ccc(-c3nc4ccccc4c4c5ccccc5c5ccccc5c34)cc2)cc1]

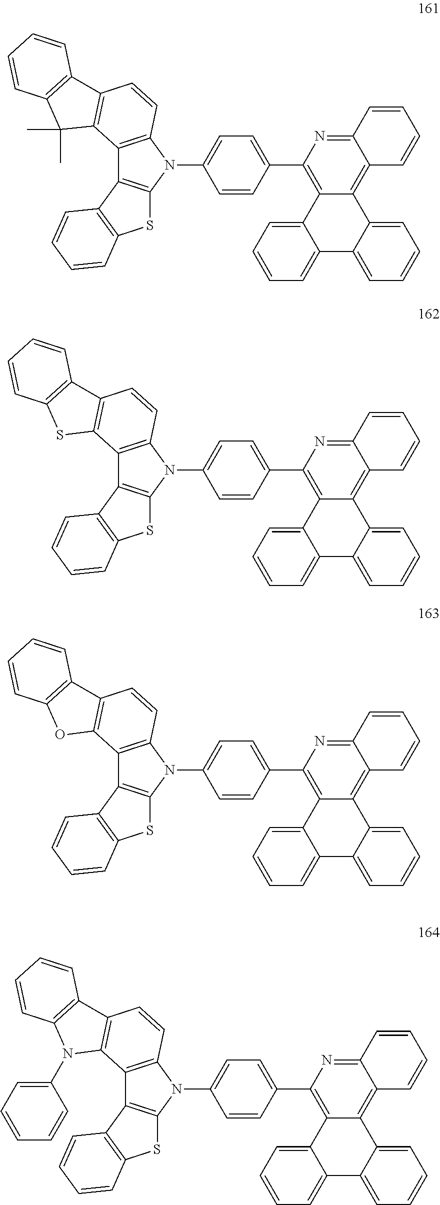 [SMILES: CC1(C)c2ccccc2-c2ccc3c(c21)c1c2ccccc2sc1n3-c1ccc(-c2nc3ccccc3c3c4ccccc4c4ccccc4c23)cc1 ; c1ccc2c(c1)nc(-c1ccc(-n3c4ccc5c6ccccc6sc5c4c4c5ccccc5sc43)cc1)c1c3ccccc3c3ccccc3c21 ; c1ccc2c(c1)nc(-c1ccc(-n3c4ccc5c6ccccc6oc5c4c4c5ccccc5sc43)cc1)c1c3ccccc3c3ccccc3c21 ; c1ccc(-n2c3ccccc3c3ccc4c(c5c6ccccc6sc5n4-c4ccc(-c5nc6ccccc6c6c7ccccc7c7ccccc7c56)cc4)c32)cc1]

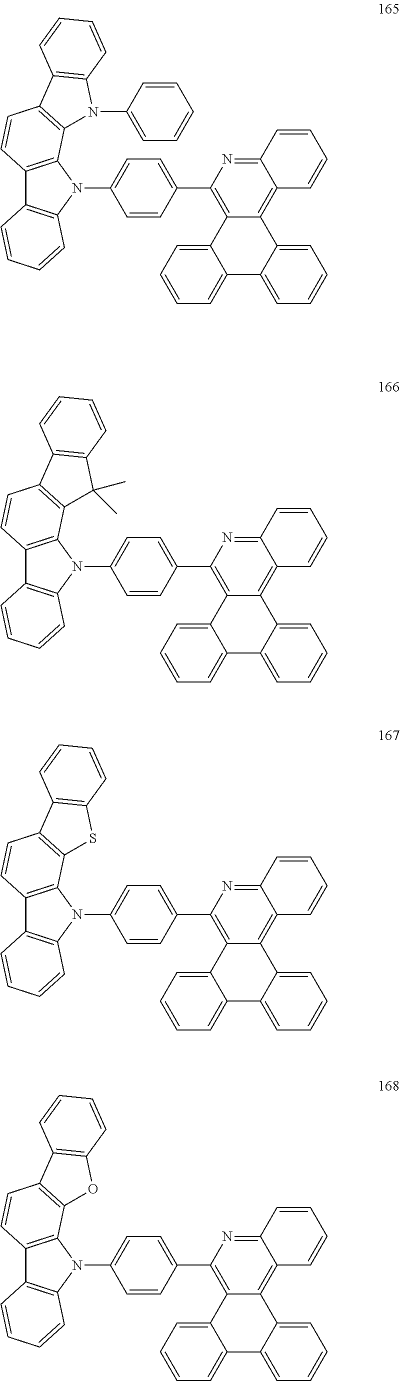 [SMILES: c1ccc(-n2c3ccccc3c3ccc4c5ccccc5n(-c5ccc(-c6nc7ccccc7c7c8ccccc8c8ccccc8c67)cc5)c4c32)cc1 ; CC1(C)c2ccccc2-c2ccc3c4ccccc4n(-c4ccc(-c5nc6ccccc6c6c7ccccc7c7ccccc7c56)cc4)c3c21 ; c1ccc2c(c1)nc(-c1ccc(-n3c4ccccc4c4ccc5c6ccccc6sc5c43)cc1)c1c3ccccc3c3ccccc3c21 ; c1ccc2c(c1)nc(-c1ccc(-n3c4ccccc4c4ccc5c6ccccc6oc5c43)cc1)c1c3ccccc3c3ccccc3c21]

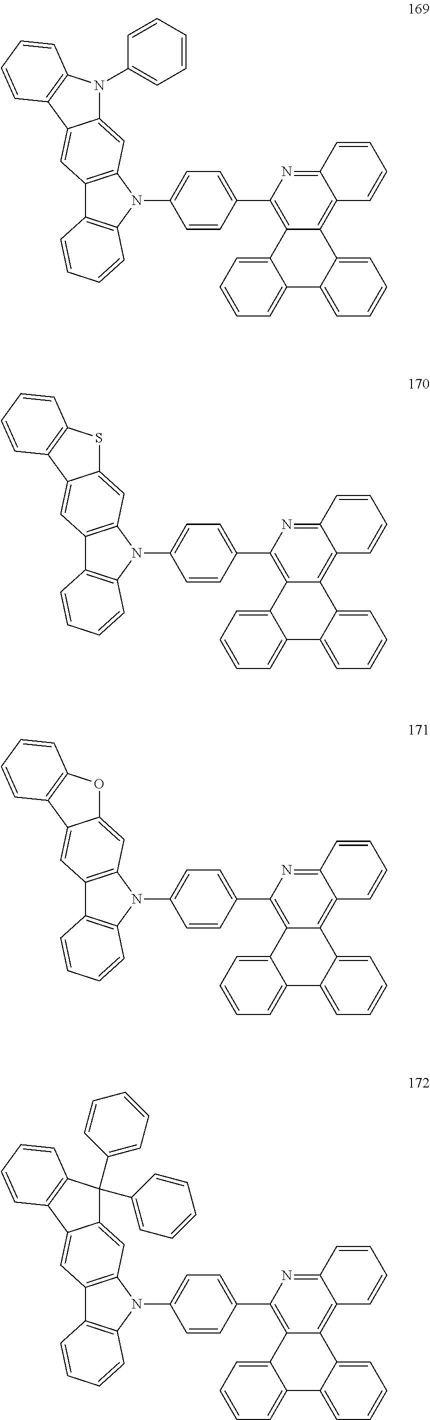 [SMILES: c1ccc(-n2c3ccccc3c3cc4c5ccccc5n(-c5ccc(-c6nc7ccccc7c7c8ccccc8c8ccccc8c67)cc5)c4cc32)cc1 ; c1ccc2c(c1)nc(-c1ccc(-n3c4ccccc4c4cc5c(cc43)sc3ccccc35)cc1)c1c3ccccc3c3ccccc3c21 ; c1ccc2c(c1)nc(-c1ccc(-n3c4ccccc4c4cc5c(cc43)oc3ccccc35)cc1)c1c3ccccc3c3ccccc3c21 ; c1ccc(C2(c3ccccc3)c3ccccc3-c3cc4c5ccccc5n(-c5ccc(-c6nc7ccccc7c7c8ccccc8c8ccccc8c67)cc5)c4cc32)cc1]

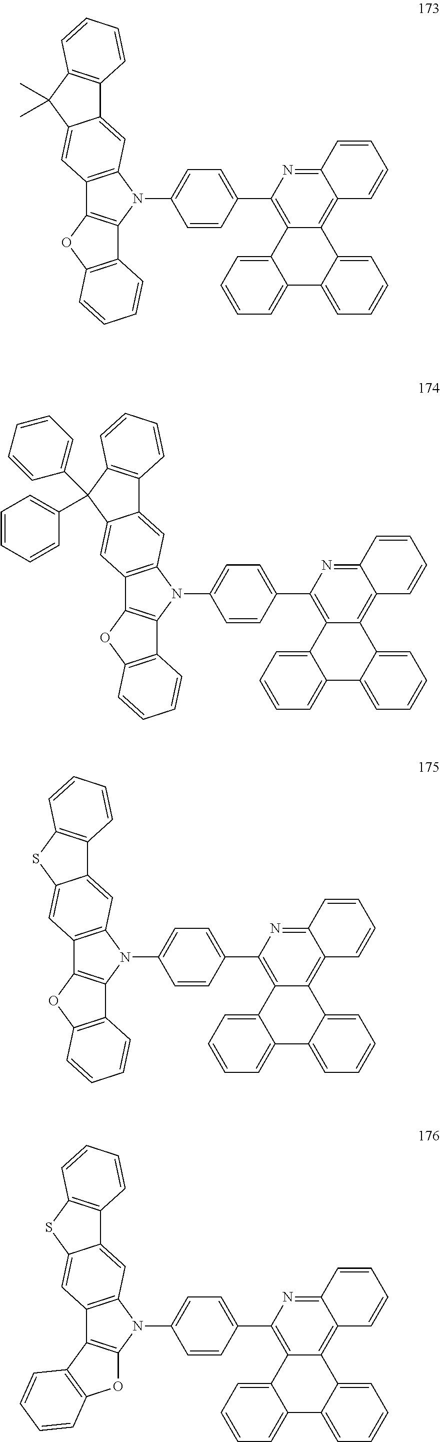 [SMILES: CC1(C)c2ccccc2-c2cc3c(cc21)c1oc2ccccc2c1n3-c1ccc(-c2nc3ccccc3c3c4ccccc4c4ccccc4c23)cc1 ; c1ccc(C2(c3ccccc3)c3ccccc3-c3cc4c(cc32)c2oc3ccccc3c2n4-c2ccc(-c3nc4ccccc4c4c5ccccc5c5ccccc5c34)cc2)cc1 ; c1ccc2c(c1)nc(-c1ccc(-n3c4cc5c(cc4c4oc6ccccc6c43)sc3ccccc35)cc1)c1c3ccccc3c3ccccc3c21 ; c1ccc2c(c1)nc(-c1ccc(-n3c4cc5c(cc4c4c6ccccc6oc43)sc3ccccc35)cc1)c1c3ccccc3c3ccccc3c21]

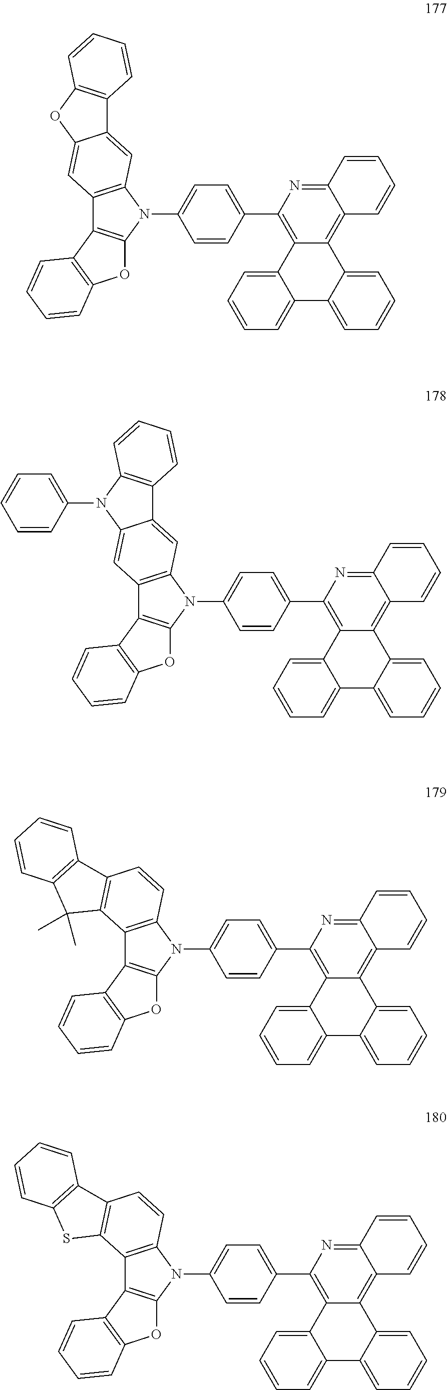 [SMILES: c1ccc2c(c1)nc(-c1ccc(-n3c4cc5c(cc4c4c6ccccc6oc43)oc3ccccc35)cc1)c1c3ccccc3c3ccccc3c21 ; c1ccc(-n2c3ccccc3c3cc4c(cc32)c2c3ccccc3oc2n4-c2ccc(-c3nc4ccccc4c4c5ccccc5c5ccccc5c34)cc2)cc1 ; CC1(C)c2ccccc2-c2ccc3c(c21)c1c2ccccc2oc1n3-c1ccc(-c2nc3ccccc3c3c4ccccc4c4ccccc4c23)cc1 ; c1ccc2c(c1)nc(-c1ccc(-n3c4ccc5c6ccccc6sc5c4c4c5ccccc5oc43)cc1)c1c3ccccc3c3ccccc3c21]

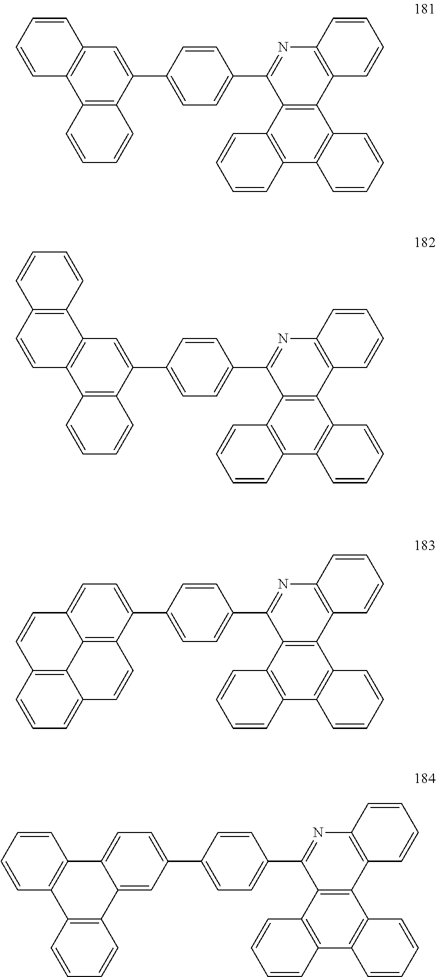 [SMILES: c1ccc2c(c1)cc(-c1ccc(-c3nc4ccccc4c4c5ccccc5c5ccccc5c34)cc1)c1ccccc12 ; c1ccc2c(c1)ccc1c3ccccc3c(-c3ccc(-c4nc5ccccc5c5c6ccccc6c6ccccc6c45)cc3)cc21 ; c1cc2ccc3ccc(-c4ccc(-c5nc6ccccc6c6c7ccccc7c7ccccc7c56)cc4)c4ccc(c1)c2c34 ; c1ccc2c(c1)nc(-c1ccc(-c3ccc4c5ccccc5c5ccccc5c4c3)cc1)c1c3ccccc3c3ccccc3c21]

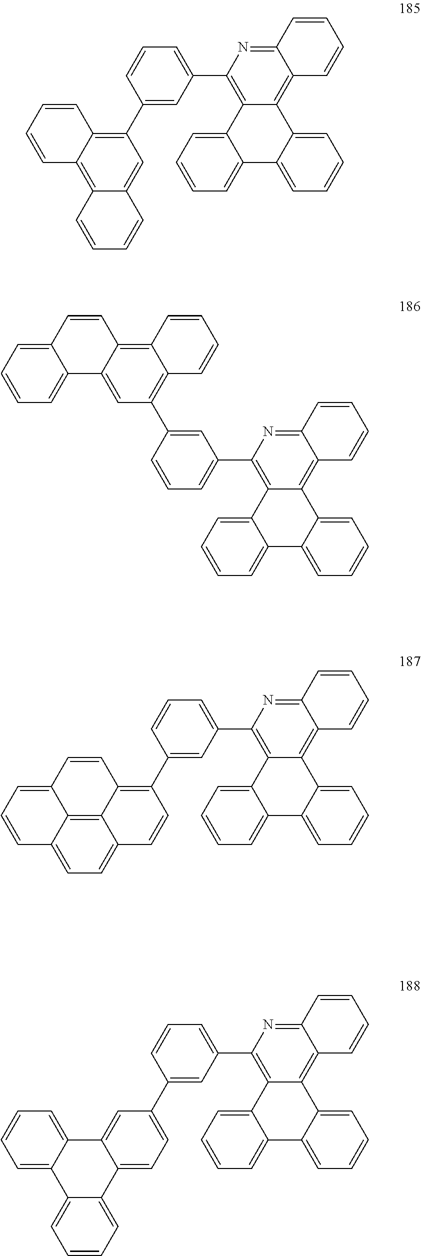 [SMILES: c1cc(-c2cc3ccccc3c3ccccc23)cc(-c2nc3ccccc3c3c4ccccc4c4ccccc4c23)c1 ; c1cc(-c2cc3c4ccccc4ccc3c3ccccc23)cc(-c2nc3ccccc3c3c4ccccc4c4ccccc4c23)c1 ; c1cc(-c2ccc3ccc4cccc5ccc2c3c45)cc(-c2nc3ccccc3c3c4ccccc4c4ccccc4c23)c1 ; c1cc(-c2ccc3c4ccccc4c4ccccc4c3c2)cc(-c2nc3ccccc3c3c4ccccc4c4ccccc4c23)c1]

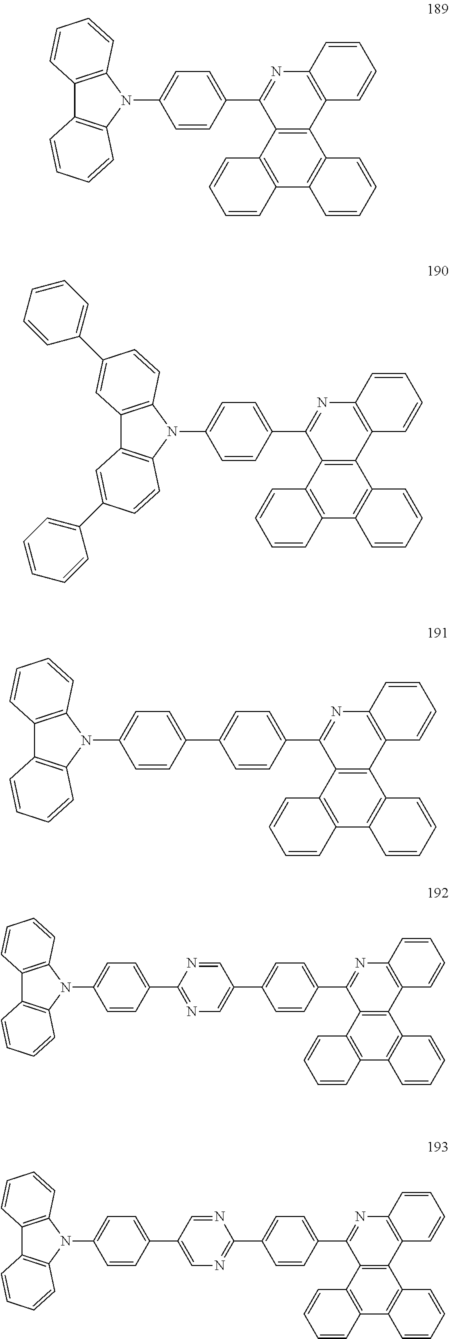 [SMILES: c1ccc2c(c1)nc(-c1ccc(-n3c4ccccc4c4ccccc43)cc1)c1c3ccccc3c3ccccc3c21 ; c1ccc(-c2ccc3c(c2)c2cc(-c4ccccc4)ccc2n3-c2ccc(-c3nc4ccccc4c4c5ccccc5c5ccccc5c34)cc2)cc1 ; c1ccc2c(c1)nc(-c1ccc(-c3ccc(-n4c5ccccc5c5ccccc54)cc3)cc1)c1c3ccccc3c3ccccc3c21 ; c1ccc2c(c1)nc(-c1ccc(-c3cnc(-c4ccc(-n5c6ccccc6c6ccccc65)cc4)nc3)cc1)c1c3ccccc3c3ccccc3c21 ; c1ccc2c(c1)nc(-c1ccc(-c3ncc(-c4ccc(-n5c6ccccc6c6ccccc65)cc4)cn3)cc1)c1c3ccccc3c3ccccc3c21]

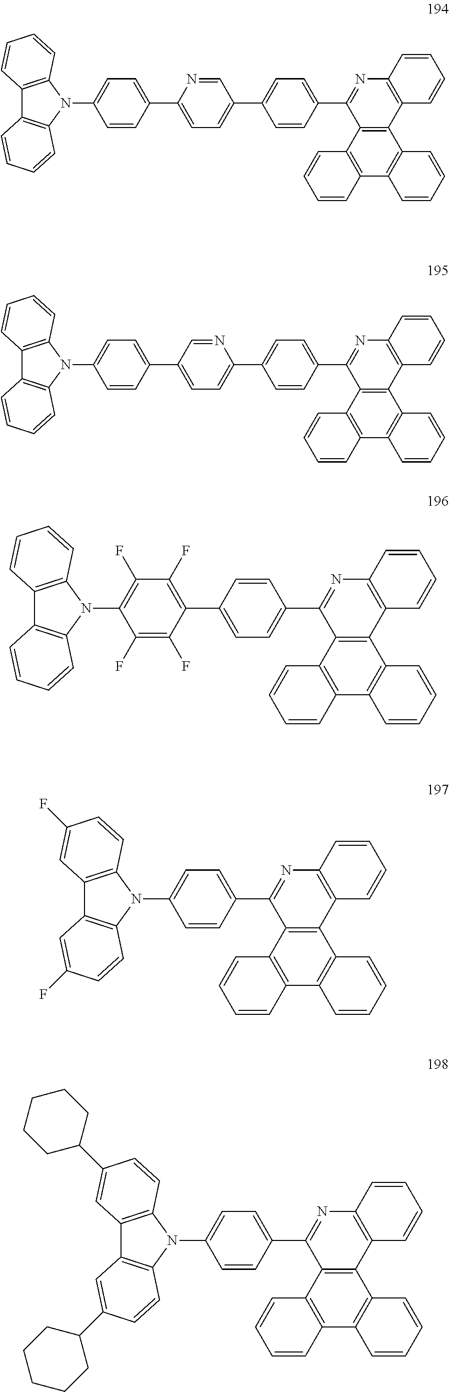 [SMILES: c1ccc2c(c1)nc(-c1ccc(-c3ccc(-c4ccc(-n5c6ccccc6c6ccccc65)cc4)nc3)cc1)c1c3ccccc3c3ccccc3c21 ; c1ccc2c(c1)nc(-c1ccc(-c3ccc(-c4ccc(-n5c6ccccc6c6ccccc65)cc4)cn3)cc1)c1c3ccccc3c3ccccc3c21 ; Fc1c(F)c(-n2c3ccccc3c3ccccc32)c(F)c(F)c1-c1ccc(-c2nc3ccccc3c3c4ccccc4c4ccccc4c23)cc1 ; Fc1ccc2c(c1)c1cc(F)ccc1n2-c1ccc(-c2nc3ccccc3c3c4ccccc4c4ccccc4c23)cc1 ; c1ccc2c(c1)nc(-c1ccc(-n3c4ccc(C5CCCCC5)cc4c4cc(C5CCCCC5)ccc43)cc1)c1c3ccccc3c3ccccc3c21]

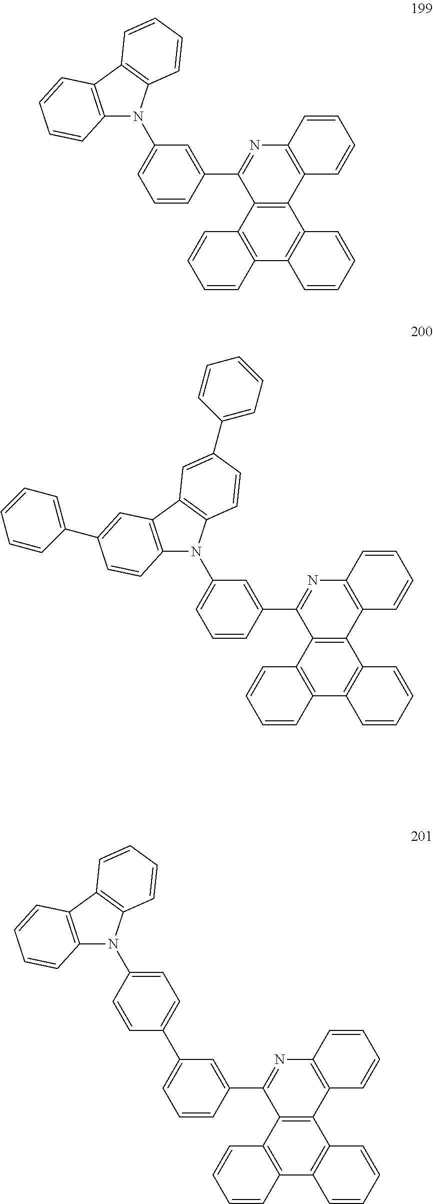 [SMILES: c1cc(-c2nc3ccccc3c3c4ccccc4c4ccccc4c23)cc(-n2c3ccccc3c3ccccc32)c1 ; c1ccc(-c2ccc3c(c2)c2cc(-c4ccccc4)ccc2n3-c2cccc(-c3nc4ccccc4c4c5ccccc5c5ccccc5c34)c2)cc1 ; c1cc(-c2ccc(-n3c4ccccc4c4ccccc43)cc2)cc(-c2nc3ccccc3c3c4ccccc4c4ccccc4c23)c1]

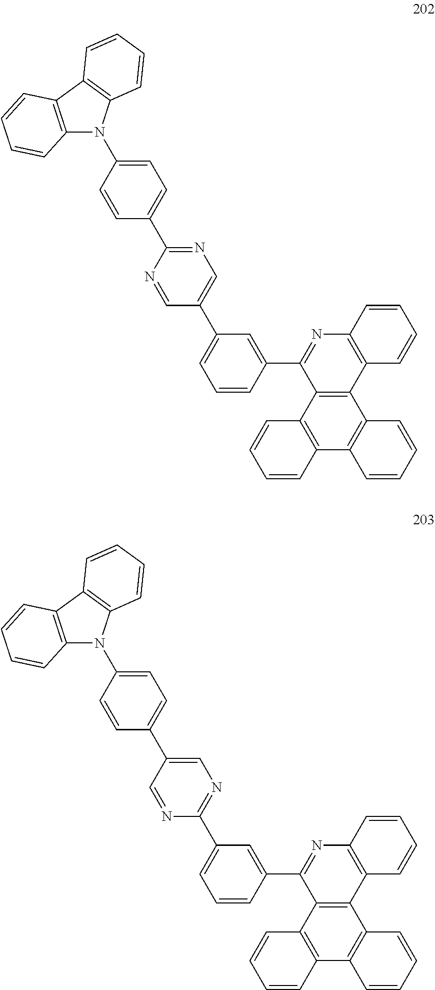 [SMILES: c1cc(-c2cnc(-c3ccc(-n4c5ccccc5c5ccccc54)cc3)nc2)cc(-c2nc3ccccc3c3c4ccccc4c4ccccc4c23)c1 ; c1cc(-c2ncc(-c3ccc(-n4c5ccccc5c5ccccc54)cc3)cn2)cc(-c2nc3ccccc3c3c4ccccc4c4ccccc4c23)c1]

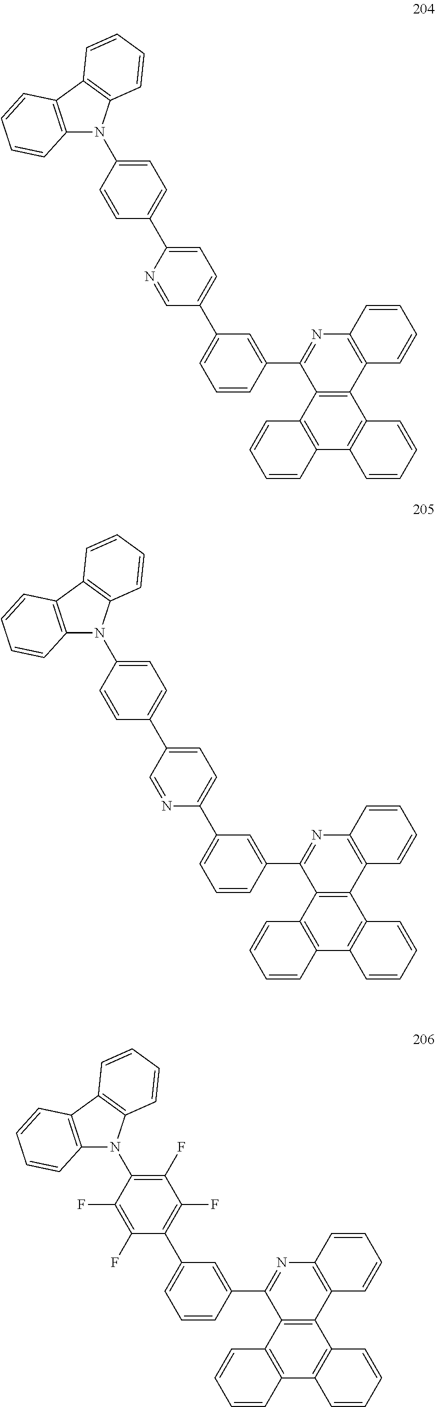 [SMILES: c1cc(-c2ccc(-c3ccc(-n4c5ccccc5c5ccccc54)cc3)nc2)cc(-c2nc3ccccc3c3c4ccccc4c4ccccc4c23)c1 ; c1cc(-c2ccc(-c3ccc(-n4c5ccccc5c5ccccc54)cc3)cn2)cc(-c2nc3ccccc3c3c4ccccc4c4ccccc4c23)c1 ; Fc1c(F)c(-n2c3ccccc3c3ccccc32)c(F)c(F)c1-c1cccc(-c2nc3ccccc3c3c4ccccc4c4ccccc4c23)c1]

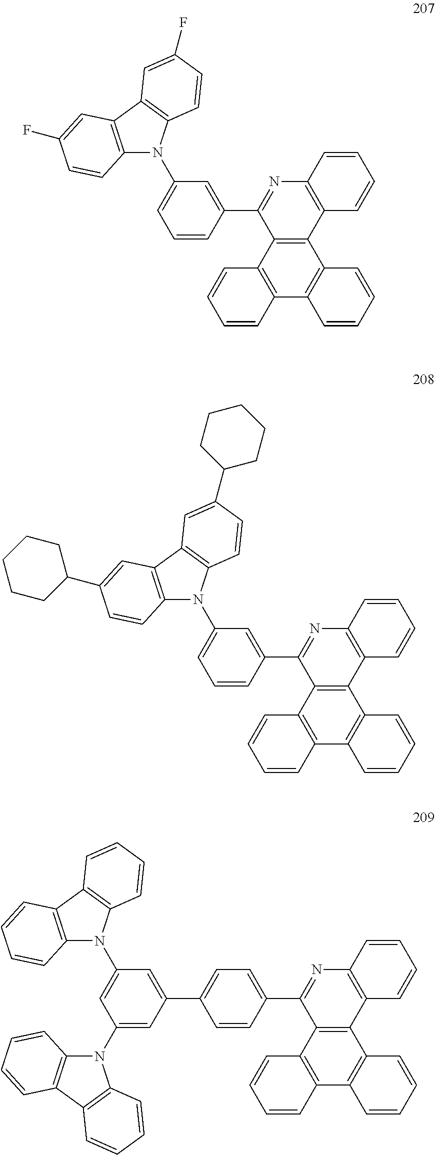 [SMILES: Fc1ccc2c(c1)c1cc(F)ccc1n2-c1cccc(-c2nc3ccccc3c3c4ccccc4c4ccccc4c23)c1 ; c1cc(-c2nc3ccccc3c3c4ccccc4c4ccccc4c23)cc(-n2c3ccc(C4CCCCC4)cc3c3cc(C4CCCCC4)ccc32)c1 ; c1ccc2c(c1)nc(-c1ccc(-c3cc(-n4c5ccccc5c5ccccc54)cc(-n4c5ccccc5c5ccccc54)c3)cc1)c1c3ccccc3c3ccccc3c21]

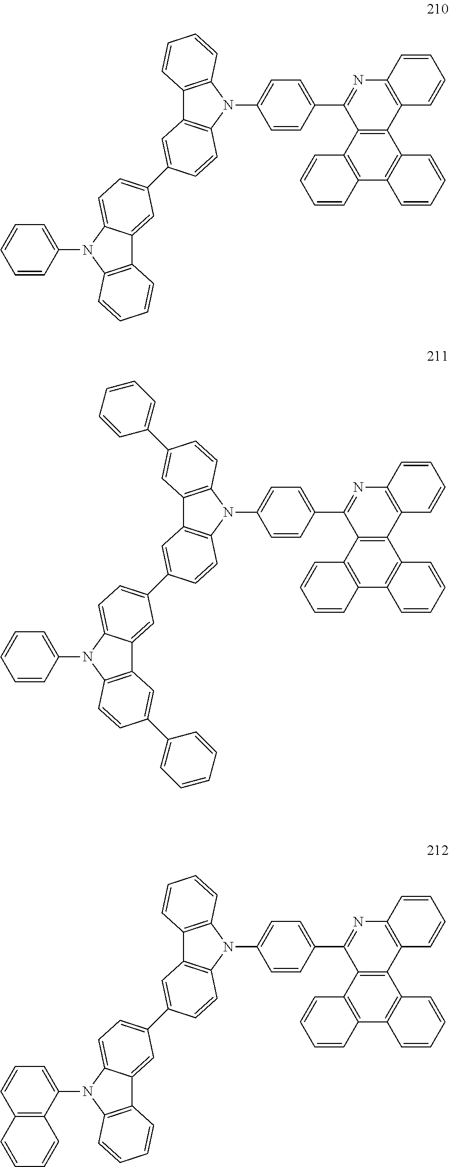 [SMILES: c1ccc(-n2c3ccccc3c3cc(-c4ccc5c(c4)c4ccccc4n5-c4ccc(-c5nc6ccccc6c6c7ccccc7c7ccccc7c56)cc4)ccc32)cc1 ; c1ccc(-c2ccc3c(c2)c2cc(-c4ccc5c(c4)c4cc(-c6ccccc6)ccc4n5-c4ccc(-c5nc6ccccc6c6c7ccccc7c7ccccc7c56)cc4)ccc2n3-c2ccccc2)cc1 ; c1ccc2c(-n3c4ccccc4c4cc(-c5ccc6c(c5)c5ccccc5n6-c5ccc(-c6nc7ccccc7c7c8ccccc8c8ccccc8c67)cc5)ccc43)cccc2c1]

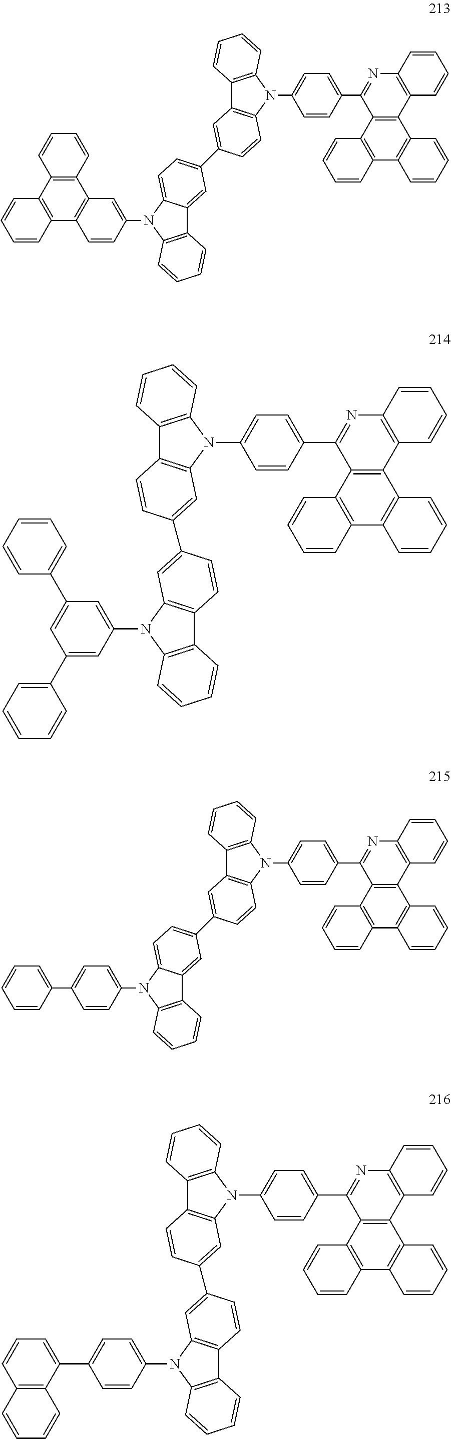 [SMILES: c1ccc2c(c1)nc(-c1ccc(-n3c4ccccc4c4cc(-c5ccc6c(c5)c5ccccc5n6-c5ccc6c7ccccc7c7ccccc7c6c5)ccc43)cc1)c1c3ccccc3c3ccccc3c21 ; c1ccc(-c2cc(-c3ccccc3)cc(-n3c4ccccc4c4ccc(-c5ccc6c7ccccc7n(-c7ccc(-c8nc9ccccc9c9c%10ccccc%10c%10ccccc%10c89)cc7)c6c5)cc43)c2)cc1 ; c1ccc(-c2ccc(-n3c4ccccc4c4cc(-c5ccc6c(c5)c5ccccc5n6-c5ccc(-c6nc7ccccc7c7c8ccccc8c8ccccc8c67)cc5)ccc43)cc2)cc1 ; c1ccc2c(-c3ccc(-n4c5ccccc5c5ccc(-c6ccc7c8ccccc8n(-c8ccc(-c9nc%10ccccc%10c%10c%11ccccc%11c%11ccccc%11c9%10)cc8)c7c6)cc54)cc3)cccc2c1]

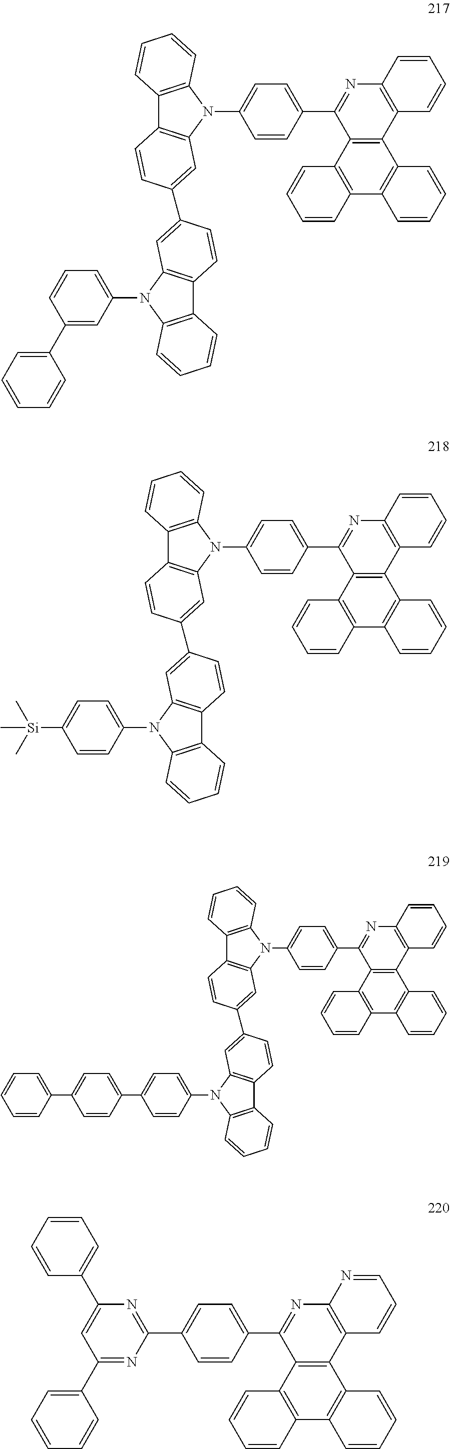 [SMILES: c1ccc(-c2cccc(-n3c4ccccc4c4ccc(-c5ccc6c7ccccc7n(-c7ccc(-c8nc9ccccc9c9c%10ccccc%10c%10ccccc%10c89)cc7)c6c5)cc43)c2)cc1 ; C[Si](C)(C)c1ccc(-n2c3ccccc3c3ccc(-c4ccc5c6ccccc6n(-c6ccc(-c7nc8ccccc8c8c9ccccc9c9ccccc9c78)cc6)c5c4)cc32)cc1 ; c1ccc(-c2ccc(-c3ccc(-n4c5ccccc5c5ccc(-c6ccc7c8ccccc8n(-c8ccc(-c9nc%10ccccc%10c%10c%11ccccc%11c%11ccccc%11c9%10)cc8)c7c6)cc54)cc3)cc2)cc1 ; c1ccc(-c2cc(-c3ccccc3)nc(-c3ccc(-c4nc5ncccc5c5c6ccccc6c6ccccc6c45)cc3)n2)cc1]

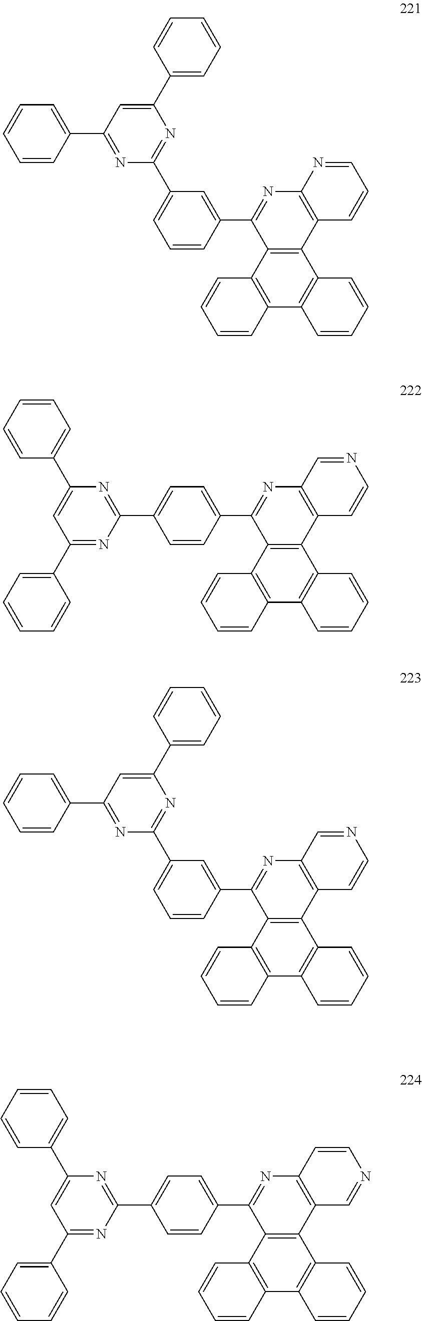 [SMILES: c1ccc(-c2cc(-c3ccccc3)nc(-c3cccc(-c4nc5ncccc5c5c6ccccc6c6ccccc6c45)c3)n2)cc1 ; c1ccc(-c2cc(-c3ccccc3)nc(-c3ccc(-c4nc5cnccc5c5c6ccccc6c6ccccc6c45)cc3)n2)cc1 ; c1ccc(-c2cc(-c3ccccc3)nc(-c3cccc(-c4nc5cnccc5c5c6ccccc6c6ccccc6c45)c3)n2)cc1 ; c1ccc(-c2cc(-c3ccccc3)nc(-c3ccc(-c4nc5ccncc5c5c6ccccc6c6ccccc6c45)cc3)n2)cc1]

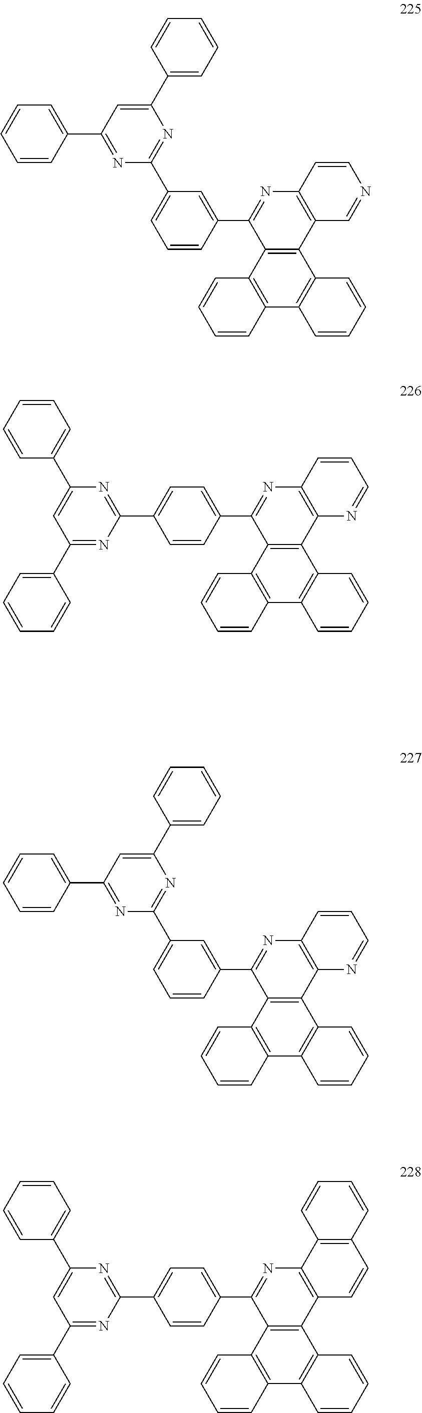 [SMILES: c1ccc(-c2cc(-c3ccccc3)nc(-c3cccc(-c4nc5ccncc5c5c6ccccc6c6ccccc6c45)c3)n2)cc1 ; c1ccc(-c2cc(-c3ccccc3)nc(-c3ccc(-c4nc5cccnc5c5c6ccccc6c6ccccc6c45)cc3)n2)cc1 ; c1ccc(-c2cc(-c3ccccc3)nc(-c3cccc(-c4nc5cccnc5c5c6ccccc6c6ccccc6c45)c3)n2)cc1 ; c1ccc(-c2cc(-c3ccccc3)nc(-c3ccc(-c4nc5c6ccccc6ccc5c5c6ccccc6c6ccccc6c45)cc3)n2)cc1]

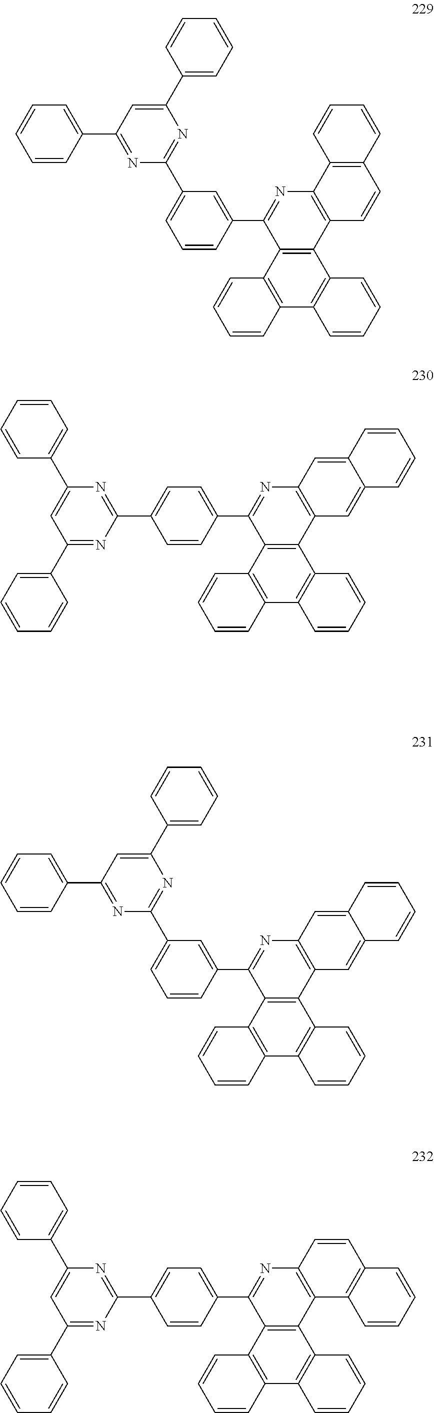 [SMILES: c1ccc(-c2cc(-c3ccccc3)nc(-c3cccc(-c4nc5c6ccccc6ccc5c5c6ccccc6c6ccccc6c45)c3)n2)cc1 ; c1ccc(-c2cc(-c3ccccc3)nc(-c3ccc(-c4nc5cc6ccccc6cc5c5c6ccccc6c6ccccc6c45)cc3)n2)cc1 ; c1ccc(-c2cc(-c3ccccc3)nc(-c3cccc(-c4nc5cc6ccccc6cc5c5c6ccccc6c6ccccc6c45)c3)n2)cc1 ; c1ccc(-c2cc(-c3ccccc3)nc(-c3ccc(-c4nc5ccc6ccccc6c5c5c6ccccc6c6ccccc6c45)cc3)n2)cc1]

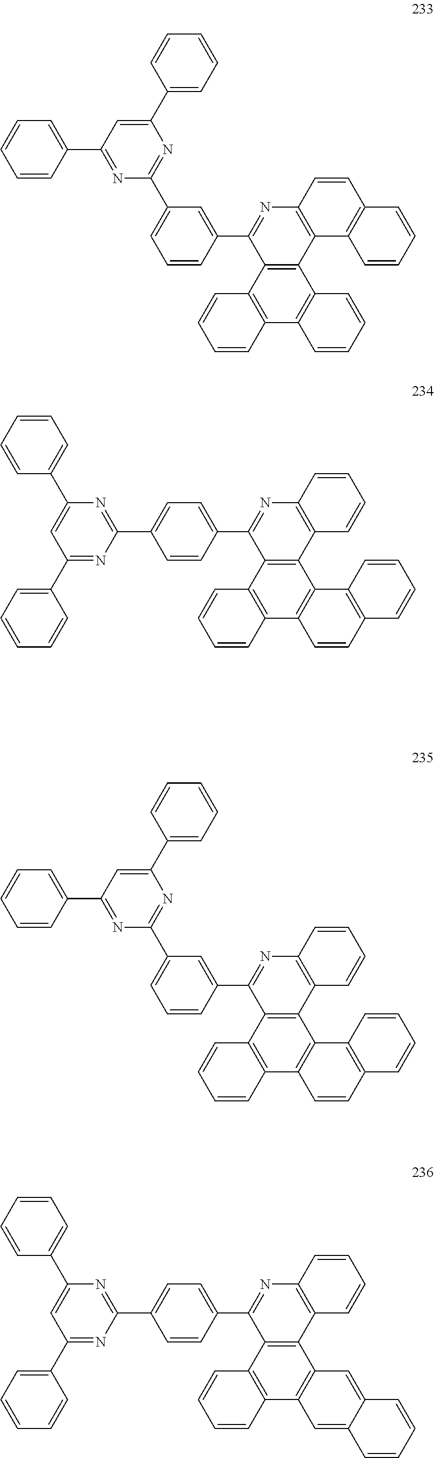 [SMILES: c1ccc(-c2cc(-c3ccccc3)nc(-c3cccc(-c4nc5ccc6ccccc6c5c5c6ccccc6c6ccccc6c45)c3)n2)cc1 ; c1ccc(-c2cc(-c3ccccc3)nc(-c3ccc(-c4nc5ccccc5c5c4c4ccccc4c4ccc6ccccc6c45)cc3)n2)cc1 ; c1ccc(-c2cc(-c3ccccc3)nc(-c3cccc(-c4nc5ccccc5c5c4c4ccccc4c4ccc6ccccc6c45)c3)n2)cc1 ; c1ccc(-c2cc(-c3ccccc3)nc(-c3ccc(-c4nc5ccccc5c5c6cc7ccccc7cc6c6ccccc6c45)cc3)n2)cc1]

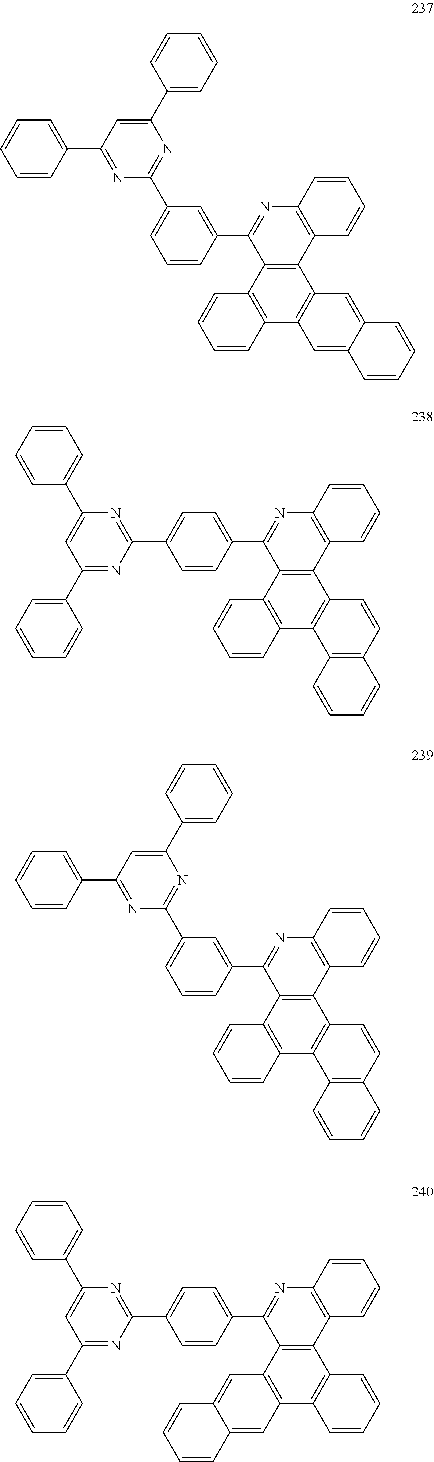 [SMILES: c1ccc(-c2cc(-c3ccccc3)nc(-c3cccc(-c4nc5ccccc5c5c6cc7ccccc7cc6c6ccccc6c45)c3)n2)cc1 ; c1ccc(-c2cc(-c3ccccc3)nc(-c3ccc(-c4nc5ccccc5c5c6ccc7ccccc7c6c6ccccc6c45)cc3)n2)cc1 ; c1ccc(-c2cc(-c3ccccc3)nc(-c3cccc(-c4nc5ccccc5c5c6ccc7ccccc7c6c6ccccc6c45)c3)n2)cc1 ; c1ccc(-c2cc(-c3ccccc3)nc(-c3ccc(-c4nc5ccccc5c5c6ccccc6c6cc7ccccc7cc6c45)cc3)n2)cc1]

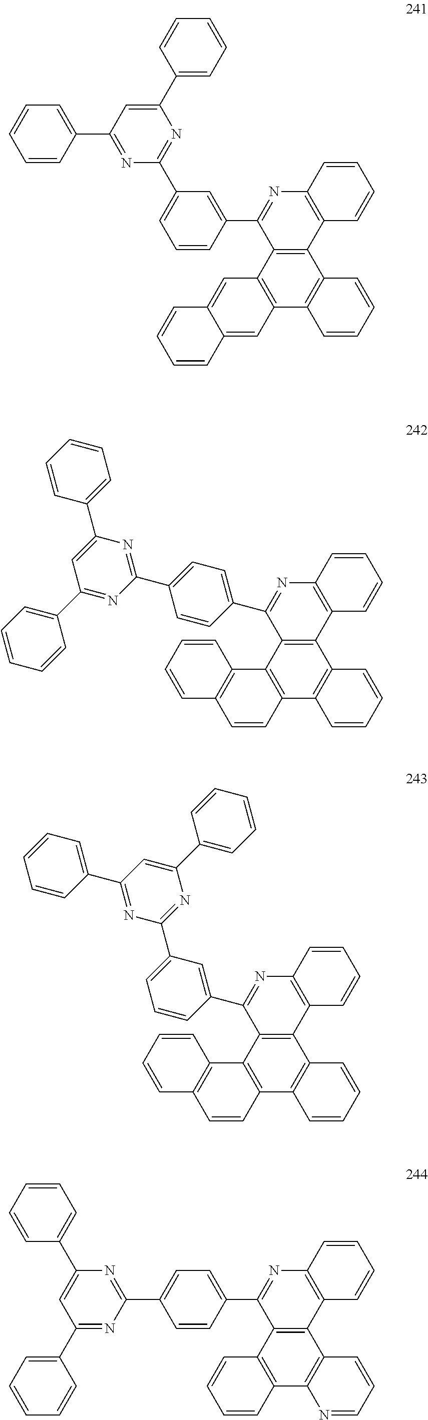 [SMILES: c1ccc(-c2cc(-c3ccccc3)nc(-c3cccc(-c4nc5ccccc5c5c6ccccc6c6cc7ccccc7cc6c45)c3)n2)cc1 ; c1ccc(-c2cc(-c3ccccc3)nc(-c3ccc(-c4nc5ccccc5c5c6ccccc6c6ccc7ccccc7c6c45)cc3)n2)cc1 ; c1ccc(-c2cc(-c3ccccc3)nc(-c3cccc(-c4nc5ccccc5c5c6ccccc6c6ccc7ccccc7c6c45)c3)n2)cc1 ; c1ccc(-c2cc(-c3ccccc3)nc(-c3ccc(-c4nc5ccccc5c5c6cccnc6c6ccccc6c45)cc3)n2)cc1]

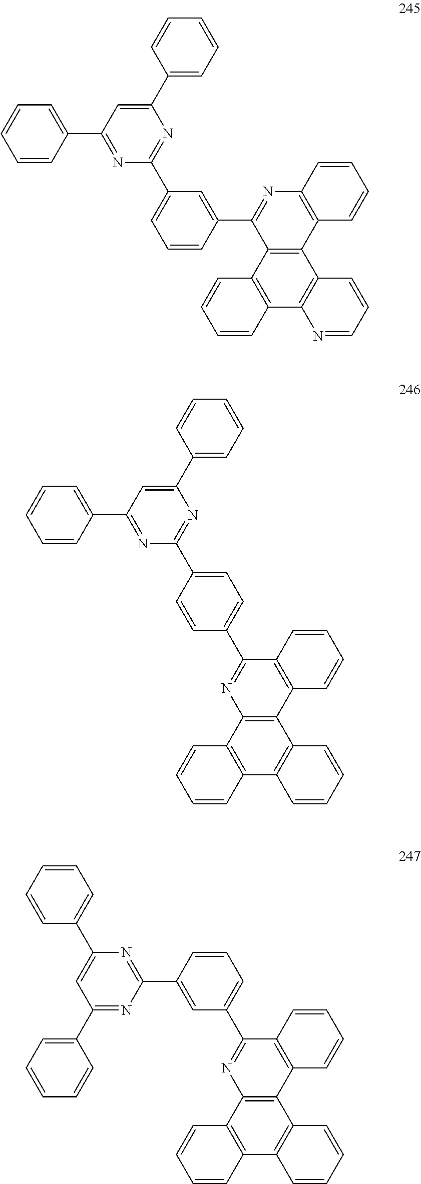 [SMILES: c1ccc(-c2cc(-c3ccccc3)nc(-c3cccc(-c4nc5ccccc5c5c6cccnc6c6ccccc6c45)c3)n2)cc1 ; c1ccc(-c2cc(-c3ccccc3)nc(-c3ccc(-c4nc5c6ccccc6c6ccccc6c5c5ccccc45)cc3)n2)cc1 ; c1ccc(-c2cc(-c3ccccc3)nc(-c3cccc(-c4nc5c6ccccc6c6ccccc6c5c5ccccc45)c3)n2)cc1]

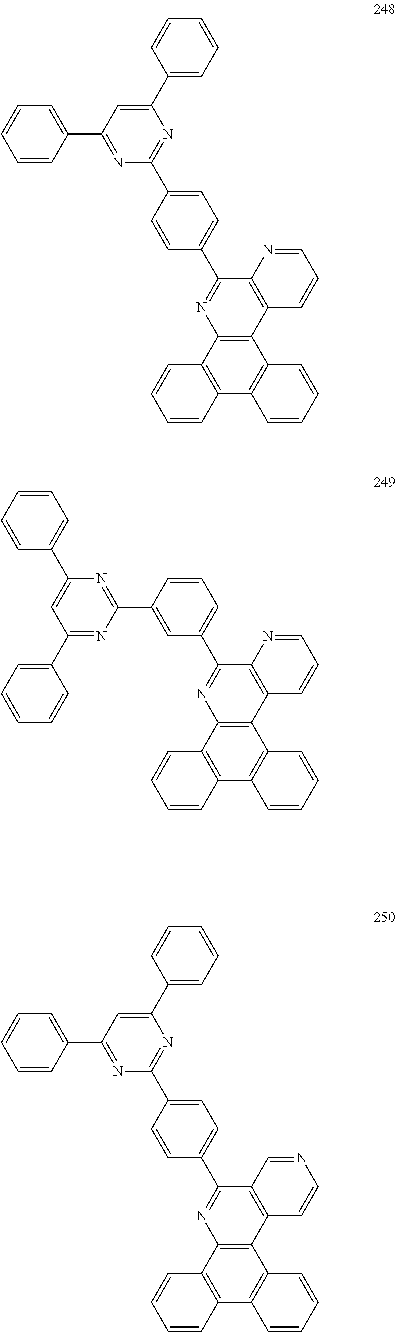 [SMILES: c1ccc(-c2cc(-c3ccccc3)nc(-c3ccc(-c4nc5c6ccccc6c6ccccc6c5c5cccnc45)cc3)n2)cc1 ; c1ccc(-c2cc(-c3ccccc3)nc(-c3cccc(-c4nc5c6ccccc6c6ccccc6c5c5cccnc45)c3)n2)cc1 ; c1ccc(-c2cc(-c3ccccc3)nc(-c3ccc(-c4nc5c6ccccc6c6ccccc6c5c5ccncc45)cc3)n2)cc1]

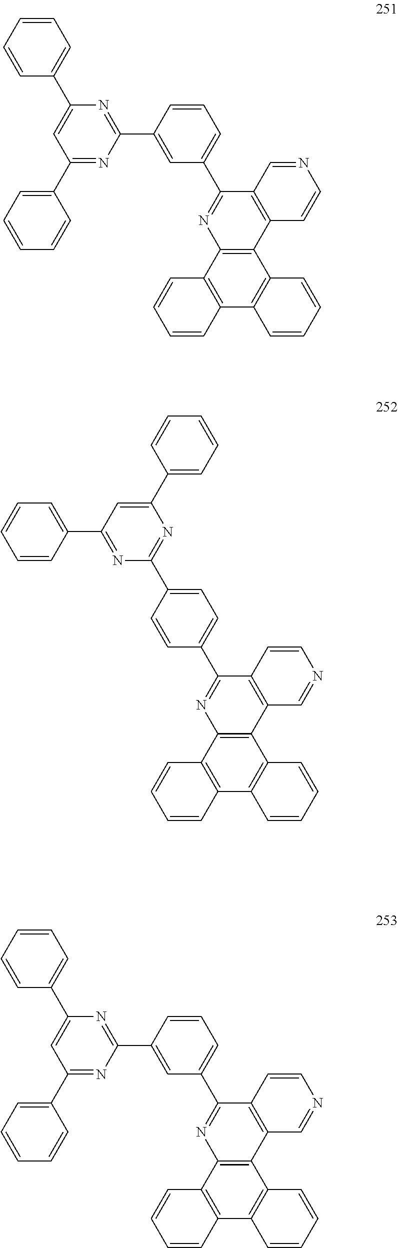 [SMILES: c1ccc(-c2cc(-c3ccccc3)nc(-c3cccc(-c4nc5c6ccccc6c6ccccc6c5c5ccncc45)c3)n2)cc1 ; c1ccc(-c2cc(-c3ccccc3)nc(-c3ccc(-c4nc5c6ccccc6c6ccccc6c5c5cnccc45)cc3)n2)cc1 ; c1ccc(-c2cc(-c3ccccc3)nc(-c3cccc(-c4nc5c6ccccc6c6ccccc6c5c5cnccc45)c3)n2)cc1]

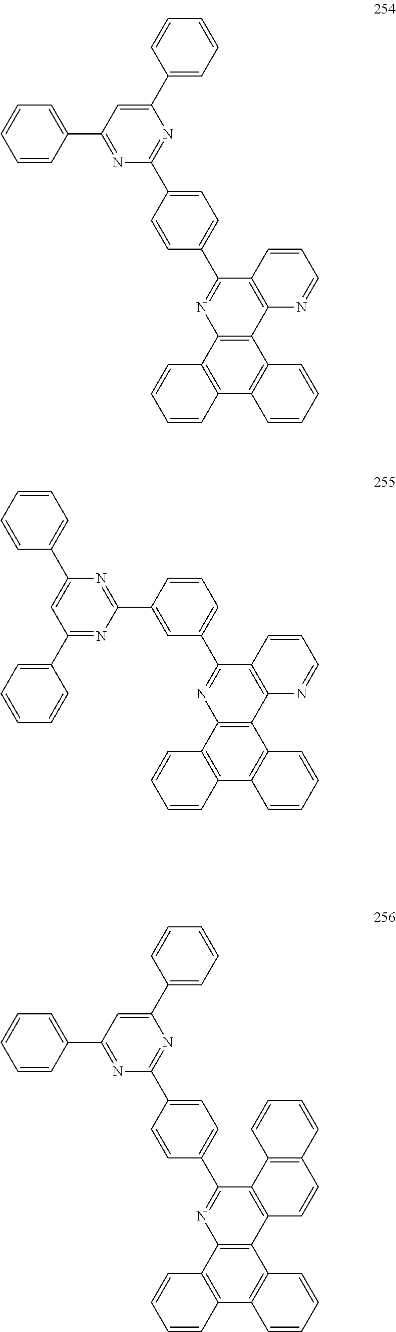 [SMILES: c1ccc(-c2cc(-c3ccccc3)nc(-c3ccc(-c4nc5c6ccccc6c6ccccc6c5c5ncccc45)cc3)n2)cc1 ; c1ccc(-c2cc(-c3ccccc3)nc(-c3cccc(-c4nc5c6ccccc6c6ccccc6c5c5ncccc45)c3)n2)cc1 ; c1ccc(-c2cc(-c3ccccc3)nc(-c3ccc(-c4nc5c6ccccc6c6ccccc6c5c5ccc6ccccc6c45)cc3)n2)cc1]

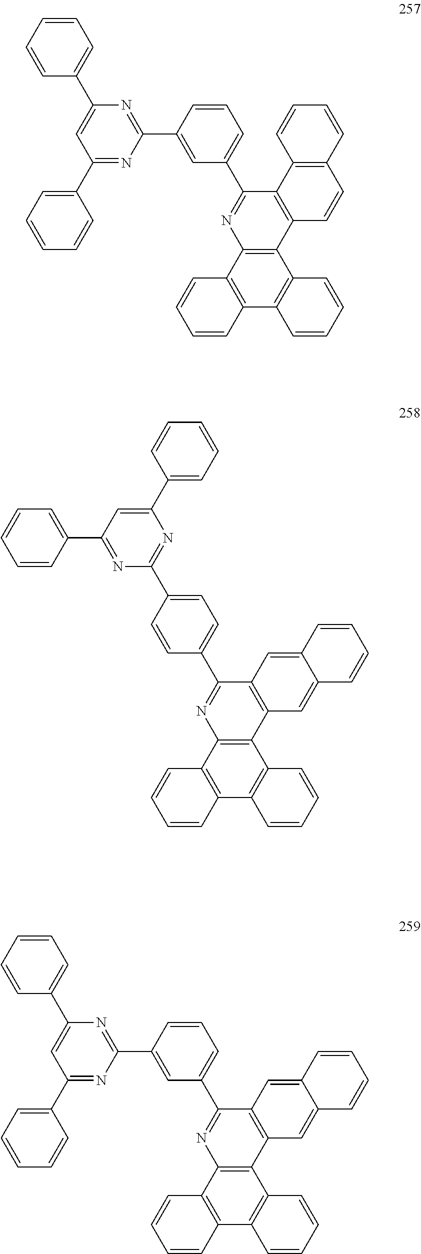 [SMILES: c1ccc(-c2cc(-c3ccccc3)nc(-c3cccc(-c4nc5c6ccccc6c6ccccc6c5c5ccc6ccccc6c45)c3)n2)cc1 ; c1ccc(-c2cc(-c3ccccc3)nc(-c3ccc(-c4nc5c6ccccc6c6ccccc6c5c5cc6ccccc6cc45)cc3)n2)cc1 ; c1ccc(-c2cc(-c3ccccc3)nc(-c3cccc(-c4nc5c6ccccc6c6ccccc6c5c5cc6ccccc6cc45)c3)n2)cc1]

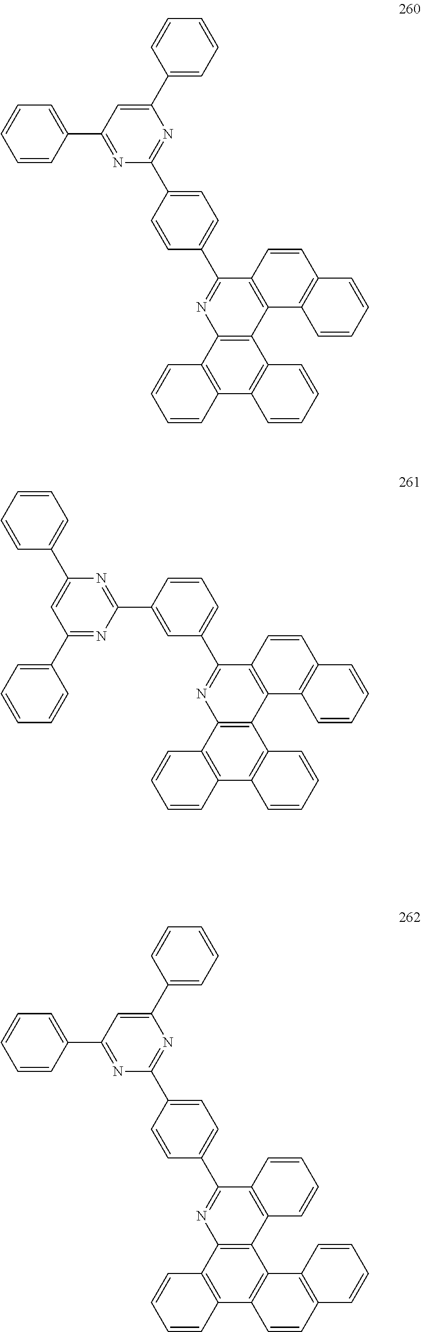 [SMILES: c1ccc(-c2cc(-c3ccccc3)nc(-c3ccc(-c4nc5c6ccccc6c6ccccc6c5c5c4ccc4ccccc45)cc3)n2)cc1 ; c1ccc(-c2cc(-c3ccccc3)nc(-c3cccc(-c4nc5c6ccccc6c6ccccc6c5c5c4ccc4ccccc45)c3)n2)cc1 ; c1ccc(-c2cc(-c3ccccc3)nc(-c3ccc(-c4nc5c6ccccc6c6ccc7ccccc7c6c5c5ccccc45)cc3)n2)cc1]

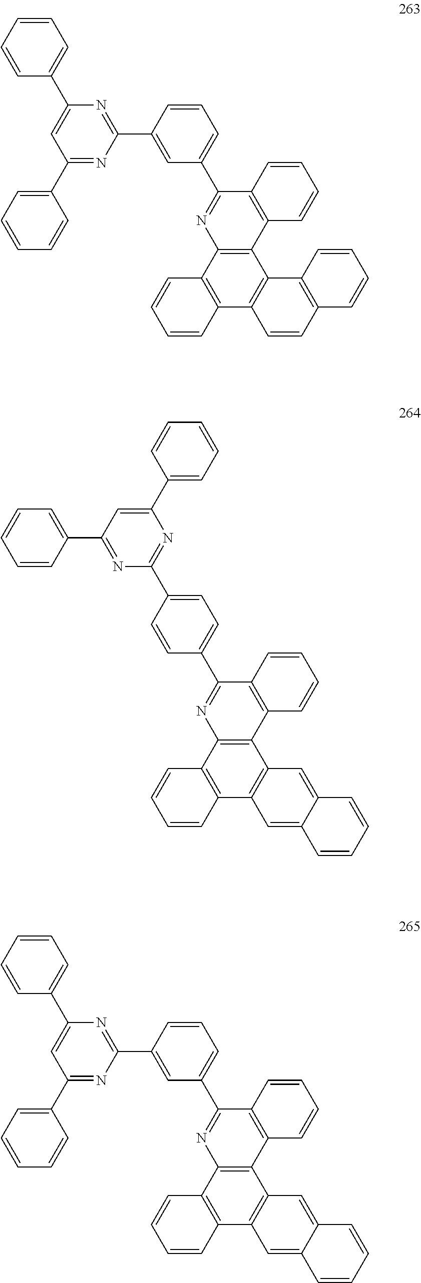 [SMILES: c1ccc(-c2cc(-c3ccccc3)nc(-c3cccc(-c4nc5c6ccccc6c6ccc7ccccc7c6c5c5ccccc45)c3)n2)cc1 ; c1ccc(-c2cc(-c3ccccc3)nc(-c3ccc(-c4nc5c6ccccc6c6cc7ccccc7cc6c5c5ccccc45)cc3)n2)cc1 ; c1ccc(-c2cc(-c3ccccc3)nc(-c3cccc(-c4nc5c6ccccc6c6cc7ccccc7cc6c5c5ccccc45)c3)n2)cc1]

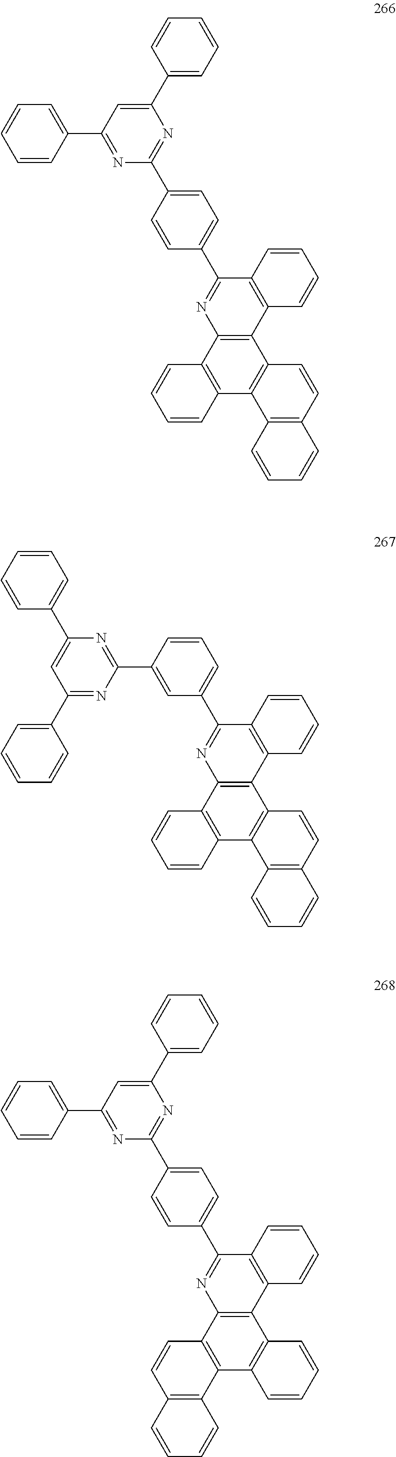 [SMILES: c1ccc(-c2cc(-c3ccccc3)nc(-c3ccc(-c4nc5c6ccccc6c6c7ccccc7ccc6c5c5ccccc45)cc3)n2)cc1 ; c1ccc(-c2cc(-c3ccccc3)nc(-c3cccc(-c4nc5c6ccccc6c6c7ccccc7ccc6c5c5ccccc45)c3)n2)cc1 ; c1ccc(-c2cc(-c3ccccc3)nc(-c3ccc(-c4nc5c6ccc7ccccc7c6c6ccccc6c5c5ccccc45)cc3)n2)cc1]

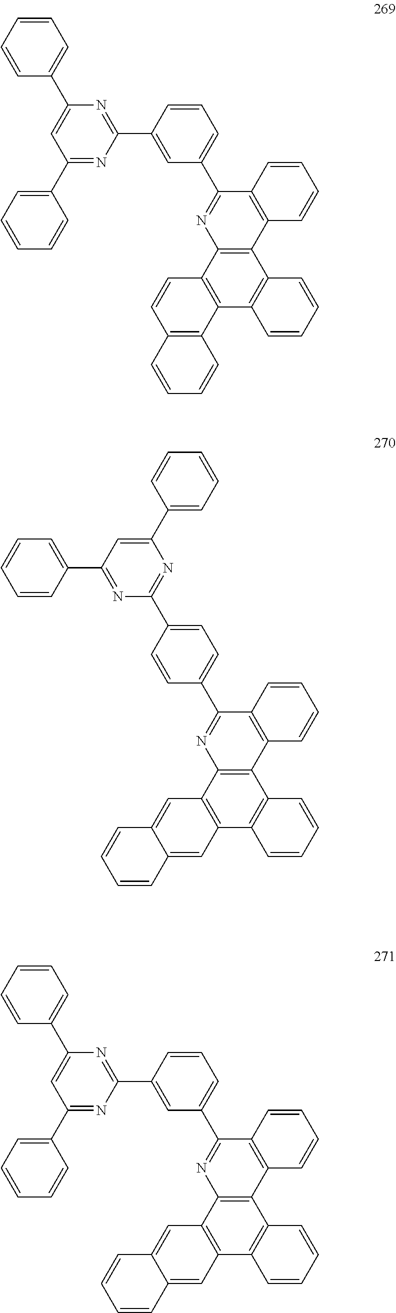 [SMILES: c1ccc(-c2cc(-c3ccccc3)nc(-c3cccc(-c4nc5c6ccc7ccccc7c6c6ccccc6c5c5ccccc45)c3)n2)cc1 ; c1ccc(-c2cc(-c3ccccc3)nc(-c3ccc(-c4nc5c6cc7ccccc7cc6c6ccccc6c5c5ccccc45)cc3)n2)cc1 ; c1ccc(-c2cc(-c3ccccc3)nc(-c3cccc(-c4nc5c6cc7ccccc7cc6c6ccccc6c5c5ccccc45)c3)n2)cc1]

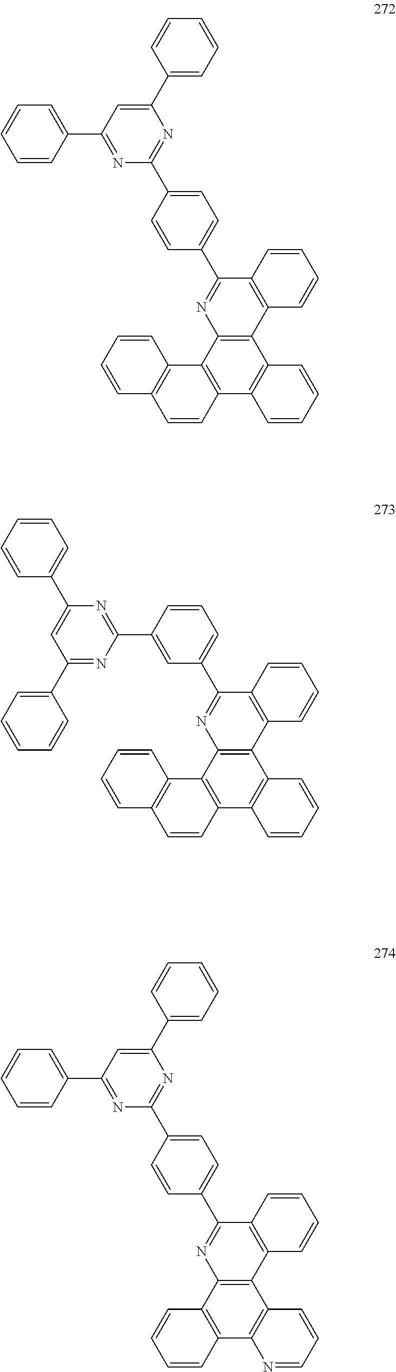 [SMILES: c1ccc(-c2cc(-c3ccccc3)nc(-c3ccc(-c4nc5c6c7ccccc7ccc6c6ccccc6c5c5ccccc45)cc3)n2)cc1 ; c1ccc(-c2cc(-c3ccccc3)nc(-c3cccc(-c4nc5c6c7ccccc7ccc6c6ccccc6c5c5ccccc45)c3)n2)cc1 ; c1ccc(-c2cc(-c3ccccc3)nc(-c3ccc(-c4nc5c6ccccc6c6ncccc6c5c5ccccc45)cc3)n2)cc1]

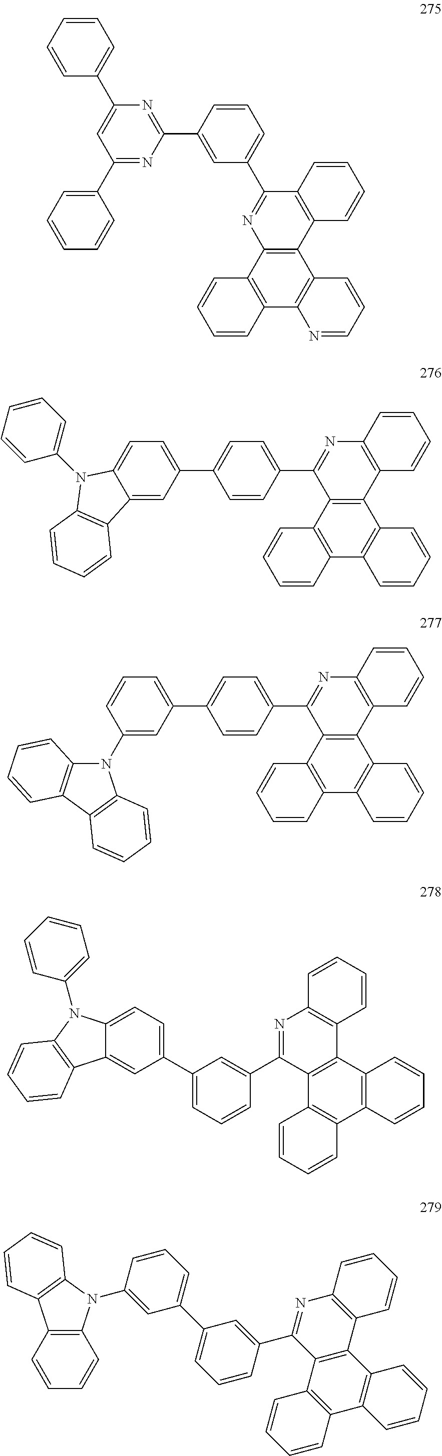 [SMILES: c1ccc(-c2cc(-c3ccccc3)nc(-c3cccc(-c4nc5c6ccccc6c6ncccc6c5c5ccccc45)c3)n2)cc1 ; c1ccc(-n2c3ccccc3c3cc(-c4ccc(-c5nc6ccccc6c6c7ccccc7c7ccccc7c56)cc4)ccc32)cc1 ; c1cc(-c2ccc(-c3nc4ccccc4c4c5ccccc5c5ccccc5c34)cc2)cc(-n2c3ccccc3c3ccccc32)c1 ; c1ccc(-n2c3ccccc3c3cc(-c4cccc(-c5nc6ccccc6c6c7ccccc7c7ccccc7c56)c4)ccc32)cc1 ; c1cc(-c2cccc(-n3c4ccccc4c4ccccc43)c2)cc(-c2nc3ccccc3c3c4ccccc4c4ccccc4c23)c1]

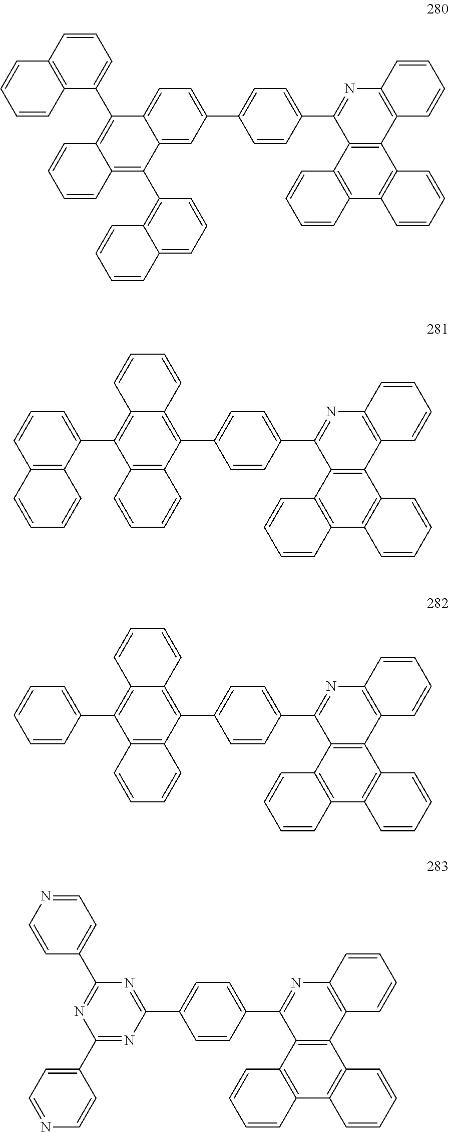 [SMILES: c1ccc2c(-c3c4ccccc4c(-c4cccc5ccccc45)c4cc(-c5ccc(-c6nc7ccccc7c7c8ccccc8c8ccccc8c67)cc5)ccc34)cccc2c1 ; c1ccc2c(-c3c4ccccc4c(-c4ccc(-c5nc6ccccc6c6c7ccccc7c7ccccc7c56)cc4)c4ccccc34)cccc2c1 ; c1ccc(-c2c3ccccc3c(-c3ccc(-c4nc5ccccc5c5c6ccccc6c6ccccc6c45)cc3)c3ccccc23)cc1 ; c1ccc2c(c1)nc(-c1ccc(-c3nc(-c4ccncc4)nc(-c4ccncc4)n3)cc1)c1c3ccccc3c3ccccc3c21]

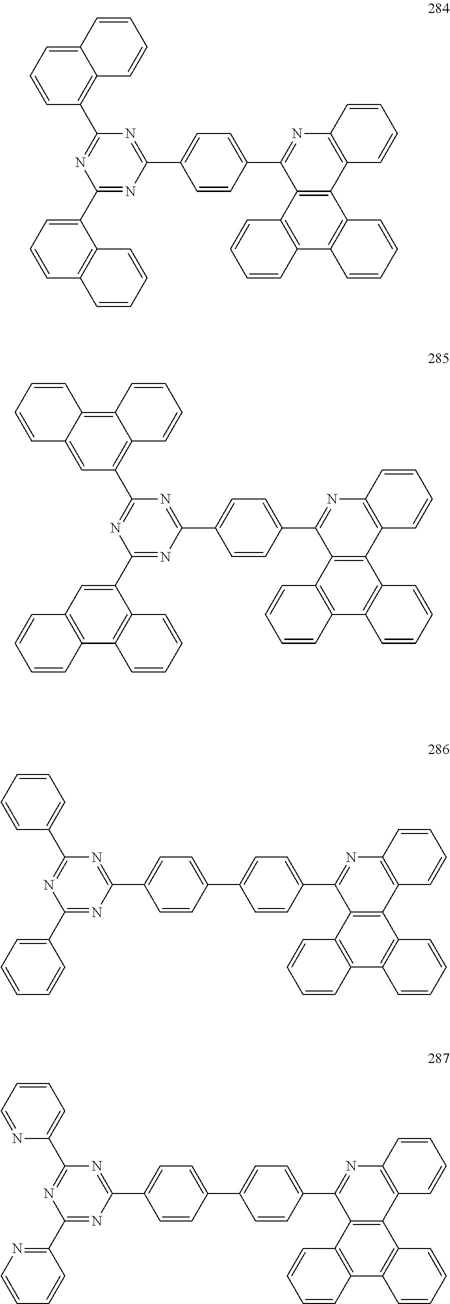 [SMILES: c1ccc2c(-c3nc(-c4ccc(-c5nc6ccccc6c6c7ccccc7c7ccccc7c56)cc4)nc(-c4cccc5ccccc45)n3)cccc2c1 ; c1ccc2c(c1)cc(-c1nc(-c3ccc(-c4nc5ccccc5c5c6ccccc6c6ccccc6c45)cc3)nc(-c3cc4ccccc4c4ccccc34)n1)c1ccccc12 ; c1ccc(-c2nc(-c3ccccc3)nc(-c3ccc(-c4ccc(-c5nc6ccccc6c6c7ccccc7c7ccccc7c56)cc4)cc3)n2)cc1 ; c1ccc(-c2nc(-c3ccc(-c4ccc(-c5nc6ccccc6c6c7ccccc7c7ccccc7c56)cc4)cc3)nc(-c3ccccn3)n2)nc1]

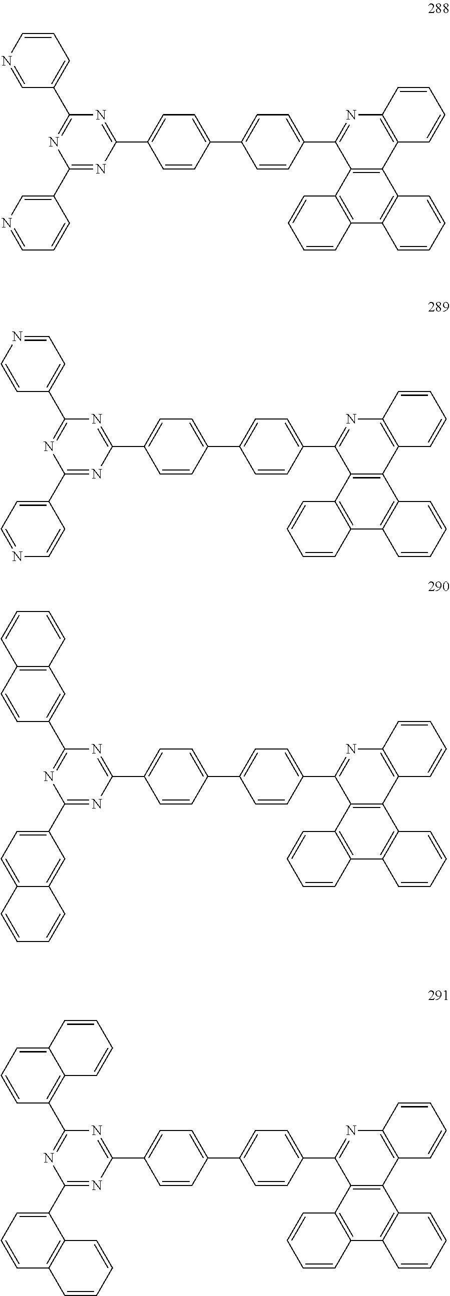 [SMILES: c1cncc(-c2nc(-c3ccc(-c4ccc(-c5nc6ccccc6c6c7ccccc7c7ccccc7c56)cc4)cc3)nc(-c3cccnc3)n2)c1 ; c1ccc2c(c1)nc(-c1ccc(-c3ccc(-c4nc(-c5ccncc5)nc(-c5ccncc5)n4)cc3)cc1)c1c3ccccc3c3ccccc3c21 ; c1ccc2cc(-c3nc(-c4ccc(-c5ccc(-c6nc7ccccc7c7c8ccccc8c8ccccc8c67)cc5)cc4)nc(-c4ccc5ccccc5c4)n3)ccc2c1 ; c1ccc2c(-c3nc(-c4ccc(-c5ccc(-c6nc7ccccc7c7c8ccccc8c8ccccc8c67)cc5)cc4)nc(-c4cccc5ccccc45)n3)cccc2c1]

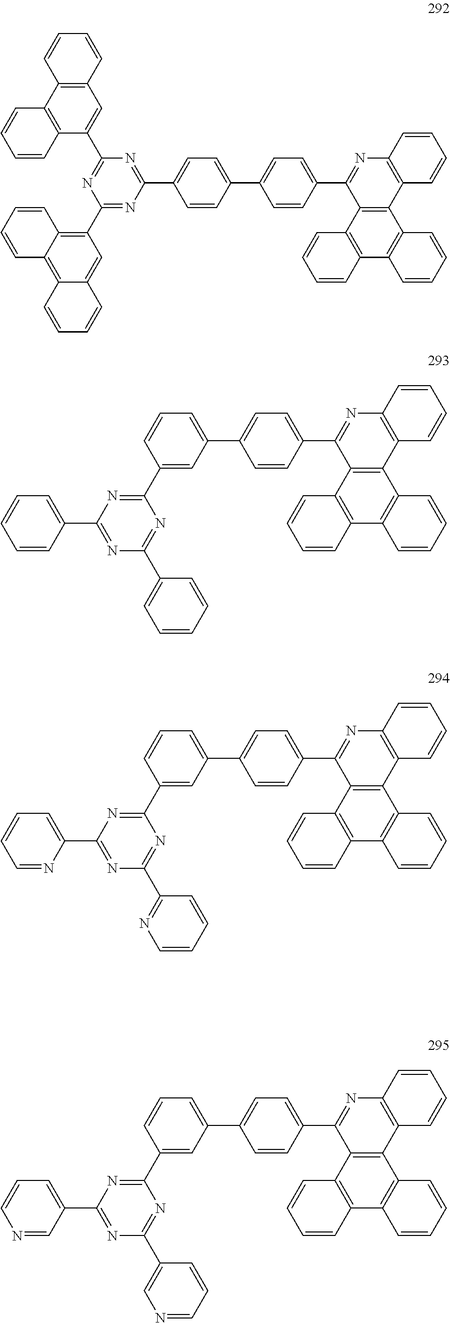 [SMILES: c1ccc2c(c1)cc(-c1nc(-c3ccc(-c4ccc(-c5nc6ccccc6c6c7ccccc7c7ccccc7c56)cc4)cc3)nc(-c3cc4ccccc4c4ccccc34)n1)c1ccccc12 ; c1ccc(-c2nc(-c3ccccc3)nc(-c3cccc(-c4ccc(-c5nc6ccccc6c6c7ccccc7c7ccccc7c56)cc4)c3)n2)cc1 ; c1ccc(-c2nc(-c3cccc(-c4ccc(-c5nc6ccccc6c6c7ccccc7c7ccccc7c56)cc4)c3)nc(-c3ccccn3)n2)nc1 ; c1cncc(-c2nc(-c3cccnc3)nc(-c3cccc(-c4ccc(-c5nc6ccccc6c6c7ccccc7c7ccccc7c56)cc4)c3)n2)c1]

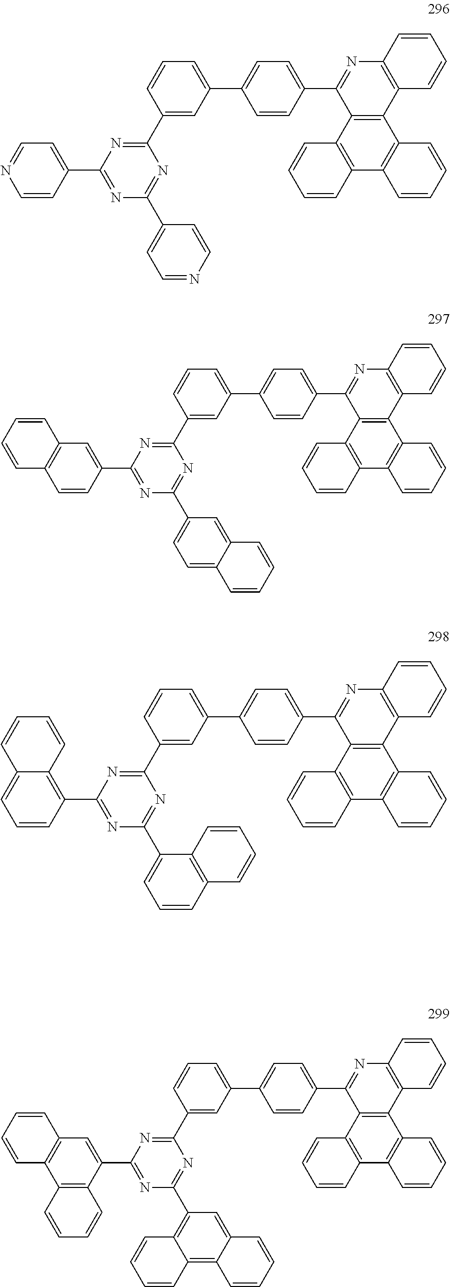 [SMILES: c1cc(-c2ccc(-c3nc4ccccc4c4c5ccccc5c5ccccc5c34)cc2)cc(-c2nc(-c3ccncc3)nc(-c3ccncc3)n2)c1 ; c1cc(-c2ccc(-c3nc4ccccc4c4c5ccccc5c5ccccc5c34)cc2)cc(-c2nc(-c3ccc4ccccc4c3)nc(-c3ccc4ccccc4c3)n2)c1 ; c1cc(-c2ccc(-c3nc4ccccc4c4c5ccccc5c5ccccc5c34)cc2)cc(-c2nc(-c3cccc4ccccc34)nc(-c3cccc4ccccc34)n2)c1 ; c1cc(-c2ccc(-c3nc4ccccc4c4c5ccccc5c5ccccc5c34)cc2)cc(-c2nc(-c3cc4ccccc4c4ccccc34)nc(-c3cc4ccccc4c4ccccc34)n2)c1]

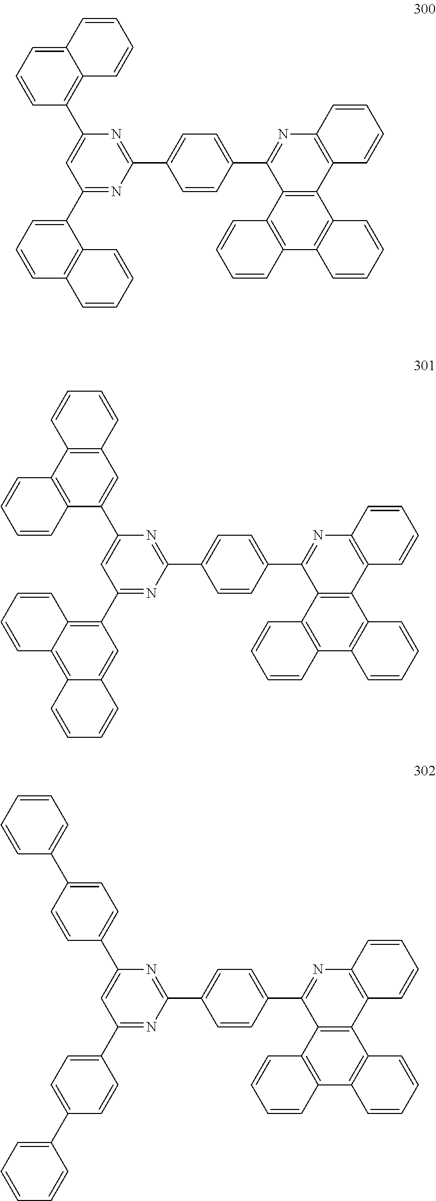 [SMILES: c1ccc2c(-c3cc(-c4cccc5ccccc45)nc(-c4ccc(-c5nc6ccccc6c6c7ccccc7c7ccccc7c56)cc4)n3)cccc2c1 ; c1ccc2c(c1)cc(-c1cc(-c3cc4ccccc4c4ccccc34)nc(-c3ccc(-c4nc5ccccc5c5c6ccccc6c6ccccc6c45)cc3)n1)c1ccccc12 ; c1ccc(-c2ccc(-c3cc(-c4ccc(-c5ccccc5)cc4)nc(-c4ccc(-c5nc6ccccc6c6c7ccccc7c7ccccc7c56)cc4)n3)cc2)cc1]

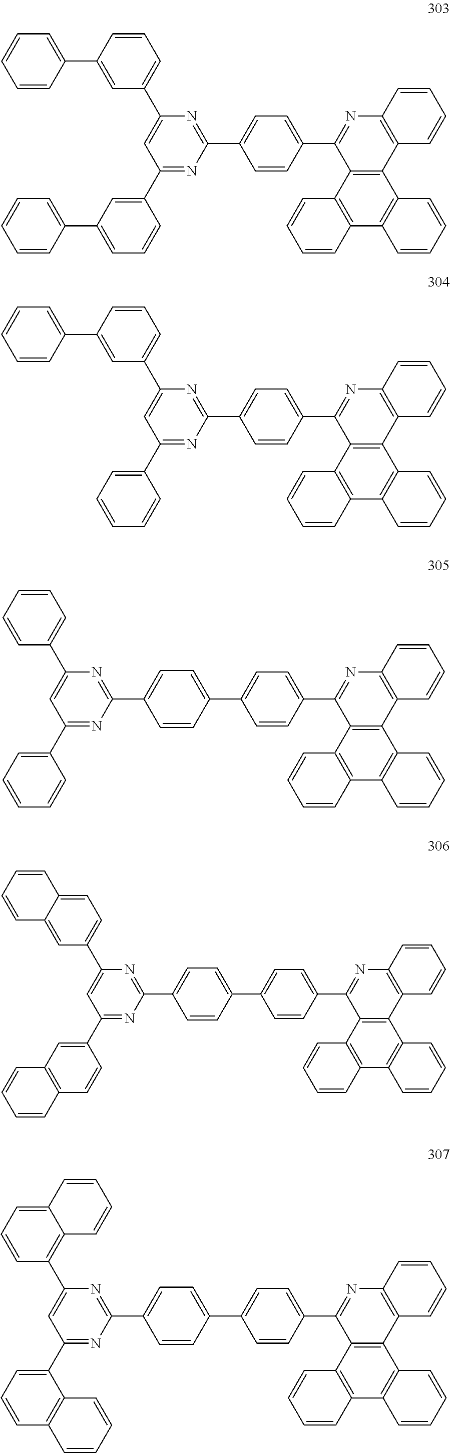 [SMILES: c1ccc(-c2cccc(-c3cc(-c4cccc(-c5ccccc5)c4)nc(-c4ccc(-c5nc6ccccc6c6c7ccccc7c7ccccc7c56)cc4)n3)c2)cc1 ; c1ccc(-c2cccc(-c3cc(-c4ccccc4)nc(-c4ccc(-c5nc6ccccc6c6c7ccccc7c7ccccc7c56)cc4)n3)c2)cc1 ; c1ccc(-c2cc(-c3ccccc3)nc(-c3ccc(-c4ccc(-c5nc6ccccc6c6c7ccccc7c7ccccc7c56)cc4)cc3)n2)cc1 ; c1ccc2cc(-c3cc(-c4ccc5ccccc5c4)nc(-c4ccc(-c5ccc(-c6nc7ccccc7c7c8ccccc8c8ccccc8c67)cc5)cc4)n3)ccc2c1 ; c1ccc2c(-c3cc(-c4cccc5ccccc45)nc(-c4ccc(-c5ccc(-c6nc7ccccc7c7c8ccccc8c8ccccc8c67)cc5)cc4)n3)cccc2c1]

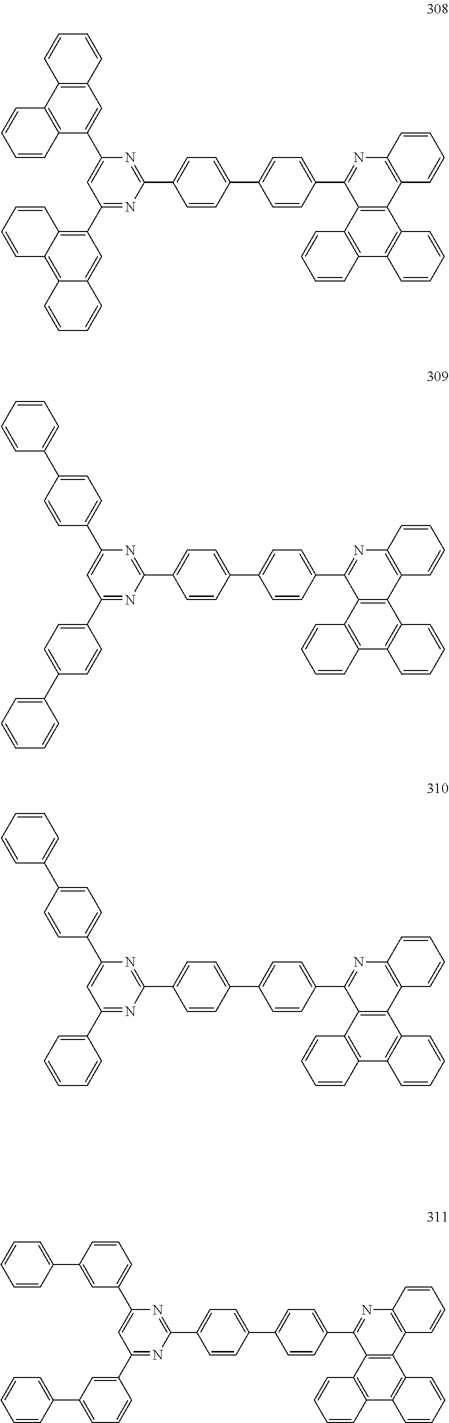 [SMILES: c1ccc2c(c1)cc(-c1cc(-c3cc4ccccc4c4ccccc34)nc(-c3ccc(-c4ccc(-c5nc6ccccc6c6c7ccccc7c7ccccc7c56)cc4)cc3)n1)c1ccccc12 ; c1ccc(-c2ccc(-c3cc(-c4ccc(-c5ccccc5)cc4)nc(-c4ccc(-c5ccc(-c6nc7ccccc7c7c8ccccc8c8ccccc8c67)cc5)cc4)n3)cc2)cc1 ; c1ccc(-c2ccc(-c3cc(-c4ccccc4)nc(-c4ccc(-c5ccc(-c6nc7ccccc7c7c8ccccc8c8ccccc8c67)cc5)cc4)n3)cc2)cc1 ; c1ccc(-c2cccc(-c3cc(-c4cccc(-c5ccccc5)c4)nc(-c4ccc(-c5ccc(-c6nc7ccccc7c7c8ccccc8c8ccccc8c67)cc5)cc4)n3)c2)cc1]

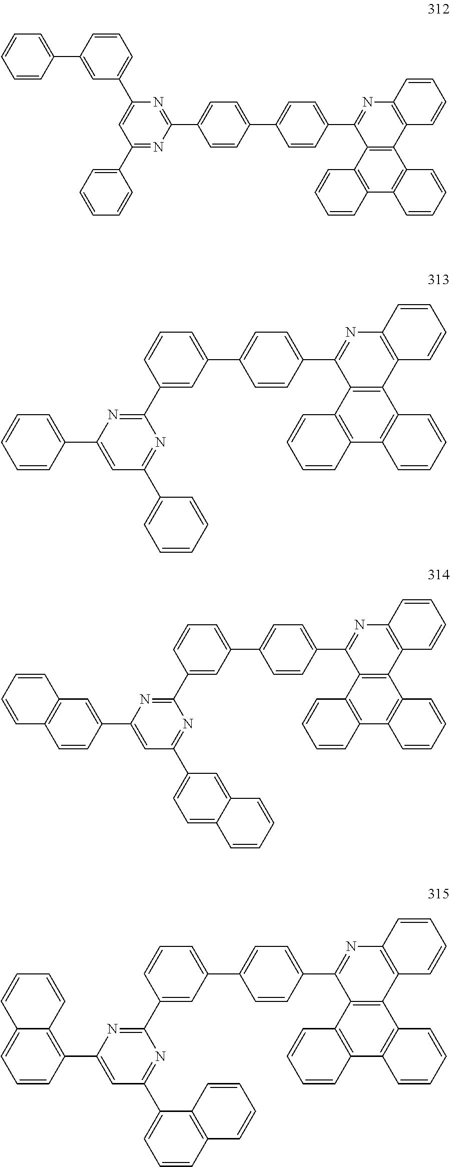 [SMILES: c1ccc(-c2cccc(-c3cc(-c4ccccc4)nc(-c4ccc(-c5ccc(-c6nc7ccccc7c7c8ccccc8c8ccccc8c67)cc5)cc4)n3)c2)cc1 ; c1ccc(-c2cc(-c3ccccc3)nc(-c3cccc(-c4ccc(-c5nc6ccccc6c6c7ccccc7c7ccccc7c56)cc4)c3)n2)cc1 ; c1cc(-c2ccc(-c3nc4ccccc4c4c5ccccc5c5ccccc5c34)cc2)cc(-c2nc(-c3ccc4ccccc4c3)cc(-c3ccc4ccccc4c3)n2)c1 ; c1cc(-c2ccc(-c3nc4ccccc4c4c5ccccc5c5ccccc5c34)cc2)cc(-c2nc(-c3cccc4ccccc34)cc(-c3cccc4ccccc34)n2)c1]

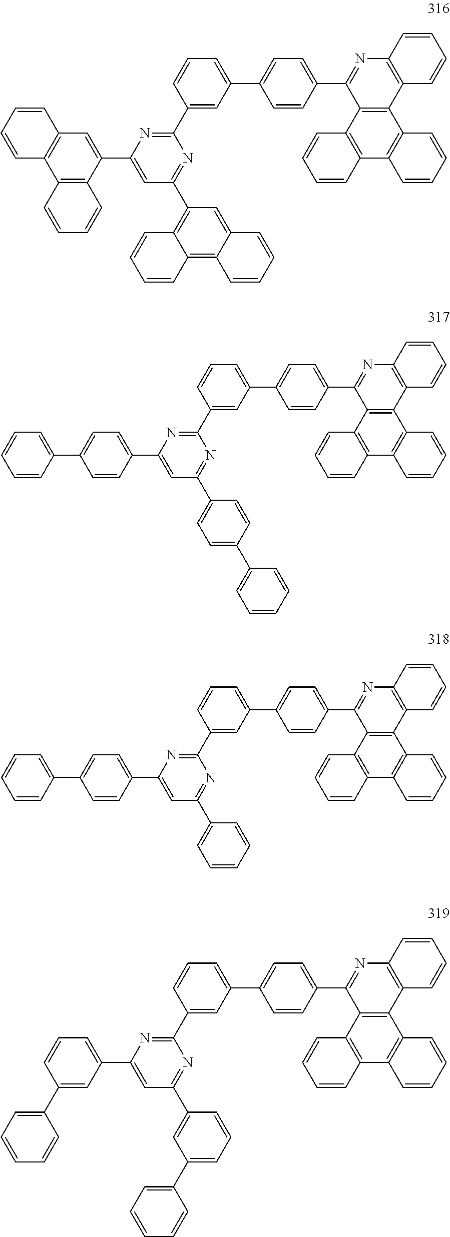 [SMILES: c1cc(-c2ccc(-c3nc4ccccc4c4c5ccccc5c5ccccc5c34)cc2)cc(-c2nc(-c3cc4ccccc4c4ccccc34)cc(-c3cc4ccccc4c4ccccc34)n2)c1 ; c1ccc(-c2ccc(-c3cc(-c4ccc(-c5ccccc5)cc4)nc(-c4cccc(-c5ccc(-c6nc7ccccc7c7c8ccccc8c8ccccc8c67)cc5)c4)n3)cc2)cc1 ; c1ccc(-c2ccc(-c3cc(-c4ccccc4)nc(-c4cccc(-c5ccc(-c6nc7ccccc7c7c8ccccc8c8ccccc8c67)cc5)c4)n3)cc2)cc1 ; c1ccc(-c2cccc(-c3cc(-c4cccc(-c5ccccc5)c4)nc(-c4cccc(-c5ccc(-c6nc7ccccc7c7c8ccccc8c8ccccc8c67)cc5)c4)n3)c2)cc1]

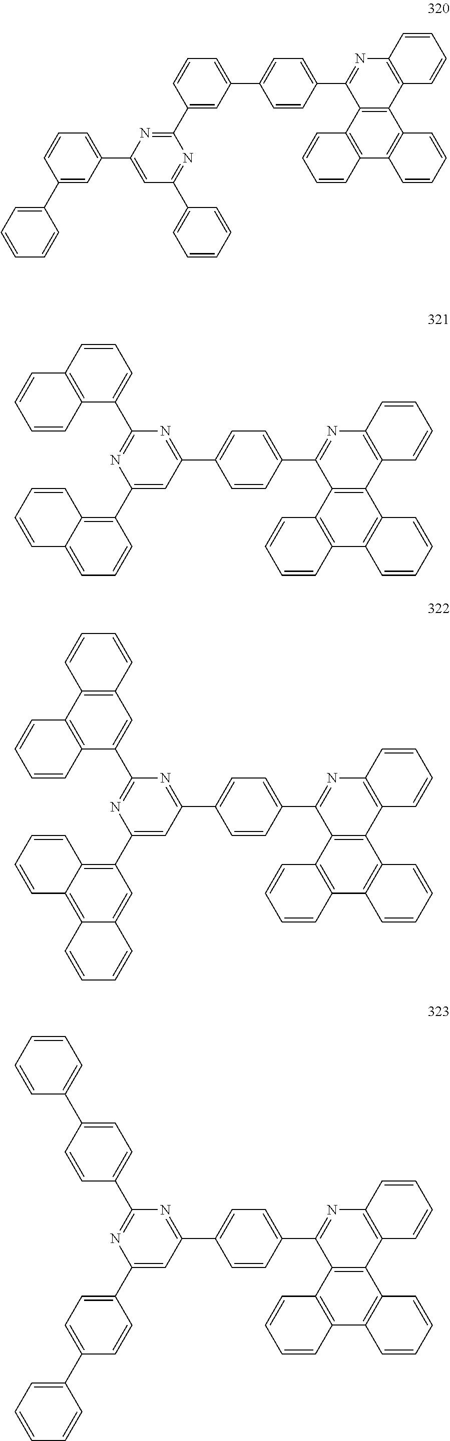 [SMILES: c1ccc(-c2cccc(-c3cc(-c4ccccc4)nc(-c4cccc(-c5ccc(-c6nc7ccccc7c7c8ccccc8c8ccccc8c67)cc5)c4)n3)c2)cc1 ; c1ccc2c(-c3cc(-c4ccc(-c5nc6ccccc6c6c7ccccc7c7ccccc7c56)cc4)nc(-c4cccc5ccccc45)n3)cccc2c1 ; c1ccc2c(c1)cc(-c1cc(-c3ccc(-c4nc5ccccc5c5c6ccccc6c6ccccc6c45)cc3)nc(-c3cc4ccccc4c4ccccc34)n1)c1ccccc12 ; c1ccc(-c2ccc(-c3cc(-c4ccc(-c5nc6ccccc6c6c7ccccc7c7ccccc7c56)cc4)nc(-c4ccc(-c5ccccc5)cc4)n3)cc2)cc1]

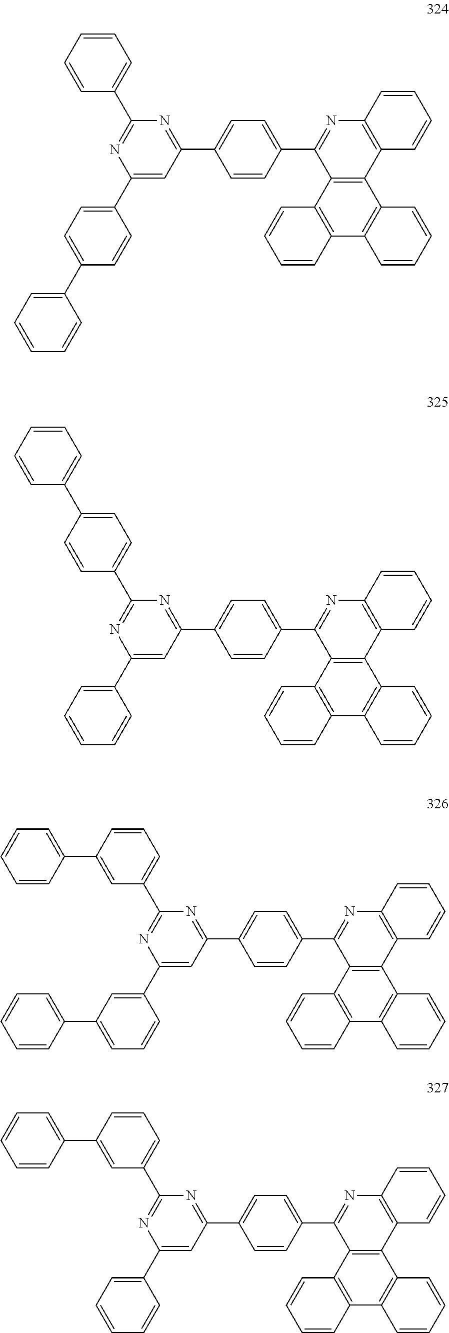 [SMILES: c1ccc(-c2ccc(-c3cc(-c4ccc(-c5nc6ccccc6c6c7ccccc7c7ccccc7c56)cc4)nc(-c4ccccc4)n3)cc2)cc1 ; c1ccc(-c2ccc(-c3nc(-c4ccccc4)cc(-c4ccc(-c5nc6ccccc6c6c7ccccc7c7ccccc7c56)cc4)n3)cc2)cc1 ; c1ccc(-c2cccc(-c3cc(-c4ccc(-c5nc6ccccc6c6c7ccccc7c7ccccc7c56)cc4)nc(-c4cccc(-c5ccccc5)c4)n3)c2)cc1 ; c1ccc(-c2cccc(-c3nc(-c4ccccc4)cc(-c4ccc(-c5nc6ccccc6c6c7ccccc7c7ccccc7c56)cc4)n3)c2)cc1]

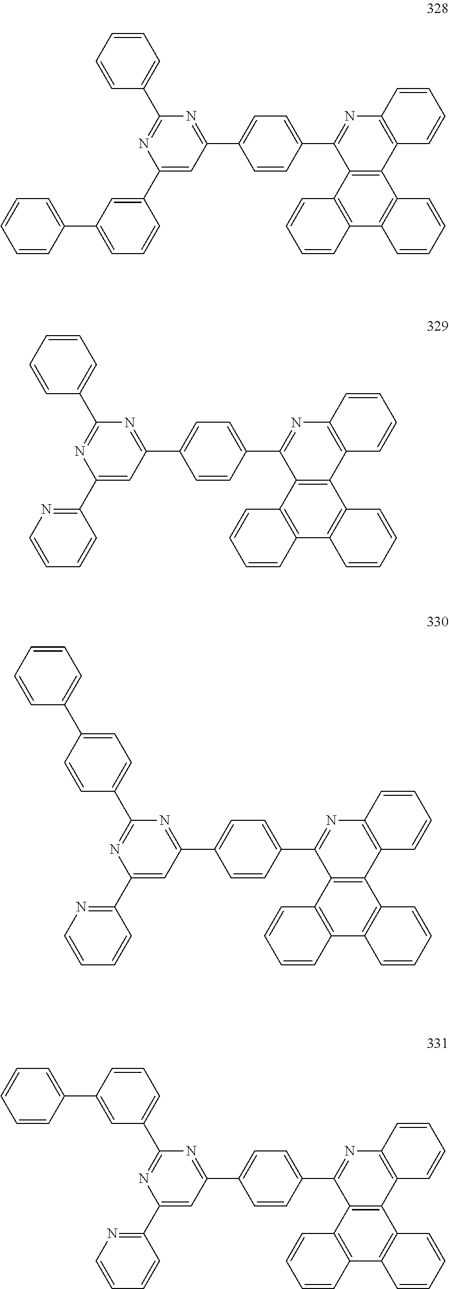 [SMILES: c1ccc(-c2cccc(-c3cc(-c4ccc(-c5nc6ccccc6c6c7ccccc7c7ccccc7c56)cc4)nc(-c4ccccc4)n3)c2)cc1 ; c1ccc(-c2nc(-c3ccc(-c4nc5ccccc5c5c6ccccc6c6ccccc6c45)cc3)cc(-c3ccccn3)n2)cc1 ; c1ccc(-c2ccc(-c3nc(-c4ccc(-c5nc6ccccc6c6c7ccccc7c7ccccc7c56)cc4)cc(-c4ccccn4)n3)cc2)cc1 ; c1ccc(-c2cccc(-c3nc(-c4ccc(-c5nc6ccccc6c6c7ccccc7c7ccccc7c56)cc4)cc(-c4ccccn4)n3)c2)cc1]

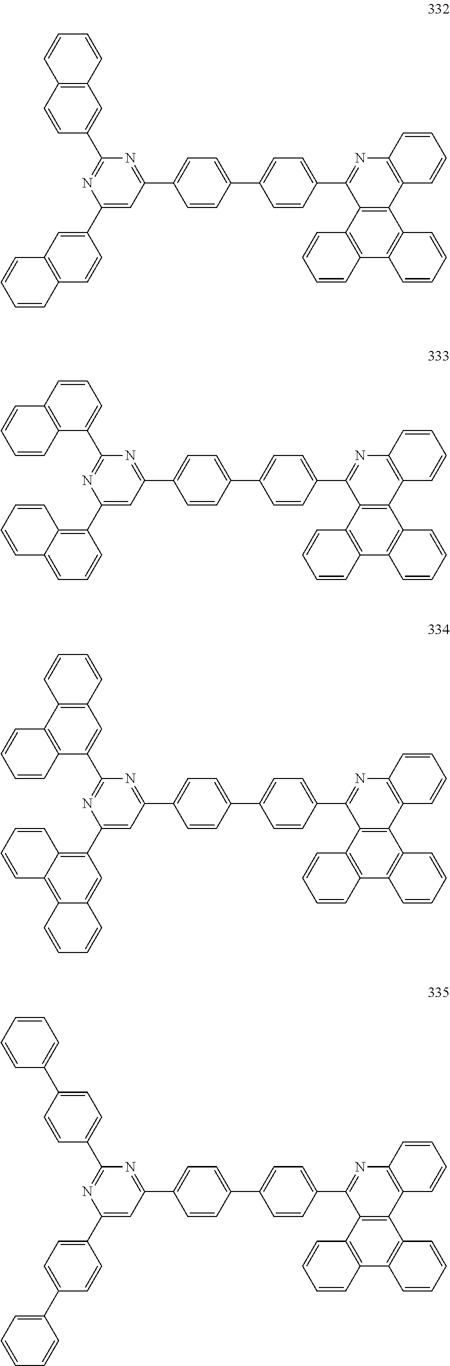 [SMILES: c1ccc2cc(-c3cc(-c4ccc(-c5ccc(-c6nc7ccccc7c7c8ccccc8c8ccccc8c67)cc5)cc4)nc(-c4ccc5ccccc5c4)n3)ccc2c1 ; c1ccc2c(-c3cc(-c4ccc(-c5ccc(-c6nc7ccccc7c7c8ccccc8c8ccccc8c67)cc5)cc4)nc(-c4cccc5ccccc45)n3)cccc2c1 ; c1ccc2c(c1)cc(-c1cc(-c3ccc(-c4ccc(-c5nc6ccccc6c6c7ccccc7c7ccccc7c56)cc4)cc3)nc(-c3cc4ccccc4c4ccccc34)n1)c1ccccc12 ; c1ccc(-c2ccc(-c3cc(-c4ccc(-c5ccc(-c6nc7ccccc7c7c8ccccc8c8ccccc8c67)cc5)cc4)nc(-c4ccc(-c5ccccc5)cc4)n3)cc2)cc1]

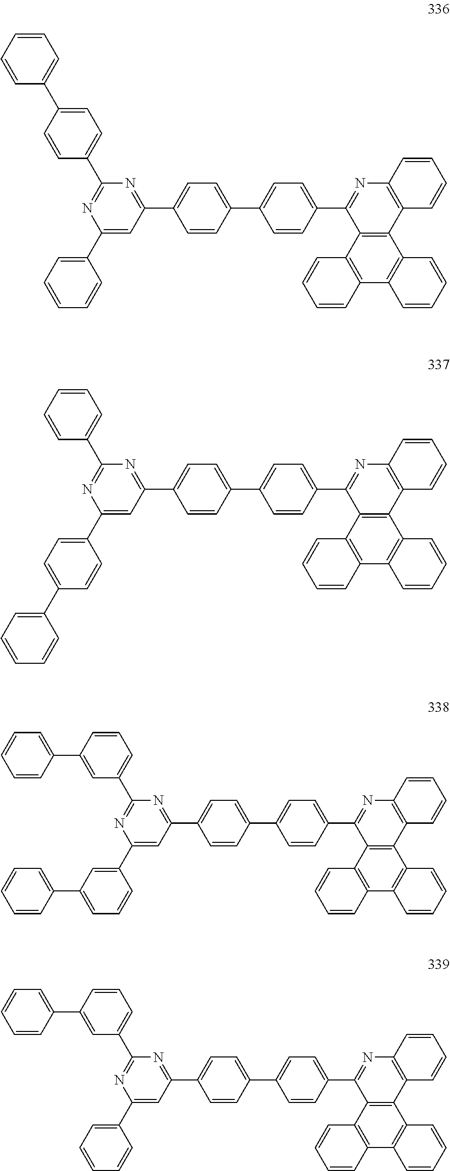 [SMILES: c1ccc(-c2ccc(-c3nc(-c4ccccc4)cc(-c4ccc(-c5ccc(-c6nc7ccccc7c7c8ccccc8c8ccccc8c67)cc5)cc4)n3)cc2)cc1 ; c1ccc(-c2ccc(-c3cc(-c4ccc(-c5ccc(-c6nc7ccccc7c7c8ccccc8c8ccccc8c67)cc5)cc4)nc(-c4ccccc4)n3)cc2)cc1 ; c1ccc(-c2cccc(-c3cc(-c4ccc(-c5ccc(-c6nc7ccccc7c7c8ccccc8c8ccccc8c67)cc5)cc4)nc(-c4cccc(-c5ccccc5)c4)n3)c2)cc1 ; c1ccc(-c2cccc(-c3nc(-c4ccccc4)cc(-c4ccc(-c5ccc(-c6nc7ccccc7c7c8ccccc8c8ccccc8c67)cc5)cc4)n3)c2)cc1]

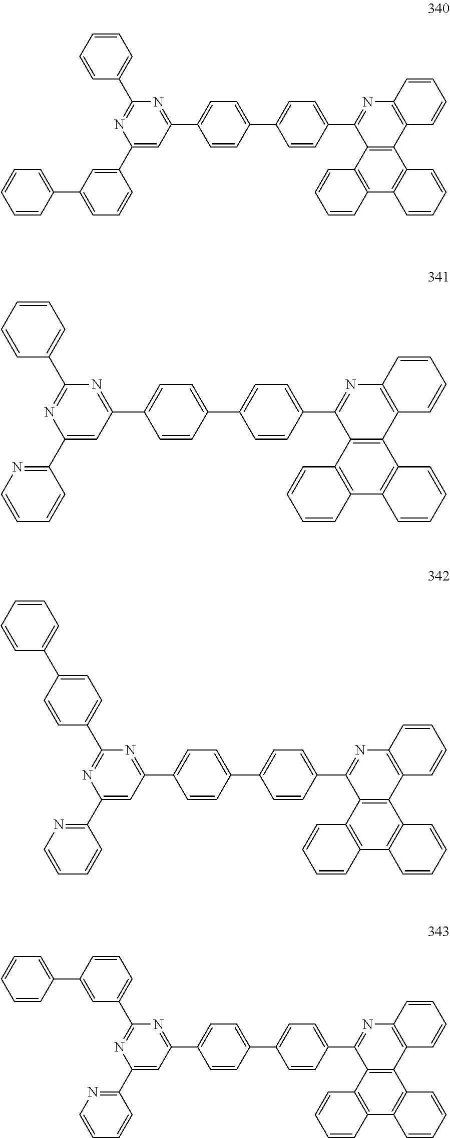 [SMILES: c1ccc(-c2cccc(-c3cc(-c4ccc(-c5ccc(-c6nc7ccccc7c7c8ccccc8c8ccccc8c67)cc5)cc4)nc(-c4ccccc4)n3)c2)cc1 ; c1ccc(-c2nc(-c3ccc(-c4ccc(-c5nc6ccccc6c6c7ccccc7c7ccccc7c56)cc4)cc3)cc(-c3ccccn3)n2)cc1 ; c1ccc(-c2ccc(-c3nc(-c4ccc(-c5ccc(-c6nc7ccccc7c7c8ccccc8c8ccccc8c67)cc5)cc4)cc(-c4ccccn4)n3)cc2)cc1 ; c1ccc(-c2cccc(-c3nc(-c4ccc(-c5ccc(-c6nc7ccccc7c7c8ccccc8c8ccccc8c67)cc5)cc4)cc(-c4ccccn4)n3)c2)cc1]

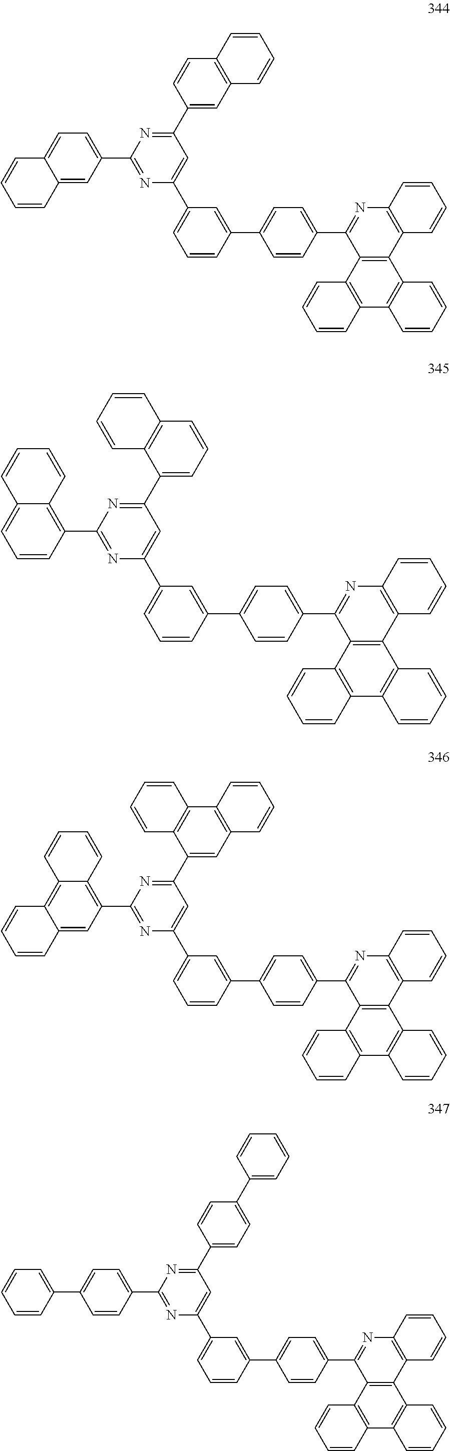 [SMILES: c1cc(-c2ccc(-c3nc4ccccc4c4c5ccccc5c5ccccc5c34)cc2)cc(-c2cc(-c3ccc4ccccc4c3)nc(-c3ccc4ccccc4c3)n2)c1 ; c1cc(-c2ccc(-c3nc4ccccc4c4c5ccccc5c5ccccc5c34)cc2)cc(-c2cc(-c3cccc4ccccc34)nc(-c3cccc4ccccc34)n2)c1 ; c1cc(-c2ccc(-c3nc4ccccc4c4c5ccccc5c5ccccc5c34)cc2)cc(-c2cc(-c3cc4ccccc4c4ccccc34)nc(-c3cc4ccccc4c4ccccc34)n2)c1 ; c1ccc(-c2ccc(-c3cc(-c4cccc(-c5ccc(-c6nc7ccccc7c7c8ccccc8c8ccccc8c67)cc5)c4)nc(-c4ccc(-c5ccccc5)cc4)n3)cc2)cc1]

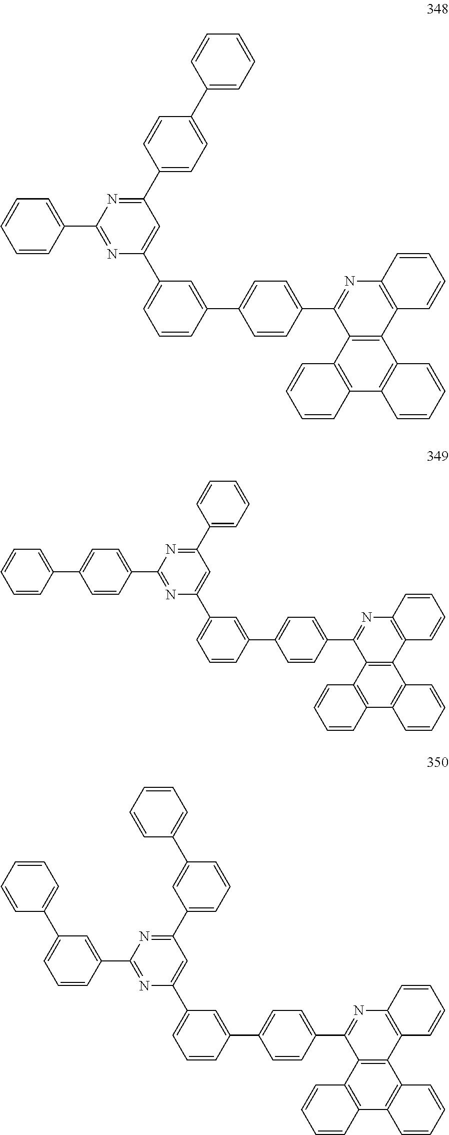 [SMILES: c1ccc(-c2ccc(-c3cc(-c4cccc(-c5ccc(-c6nc7ccccc7c7c8ccccc8c8ccccc8c67)cc5)c4)nc(-c4ccccc4)n3)cc2)cc1 ; c1ccc(-c2ccc(-c3nc(-c4ccccc4)cc(-c4cccc(-c5ccc(-c6nc7ccccc7c7c8ccccc8c8ccccc8c67)cc5)c4)n3)cc2)cc1 ; c1ccc(-c2cccc(-c3cc(-c4cccc(-c5ccc(-c6nc7ccccc7c7c8ccccc8c8ccccc8c67)cc5)c4)nc(-c4cccc(-c5ccccc5)c4)n3)c2)cc1]

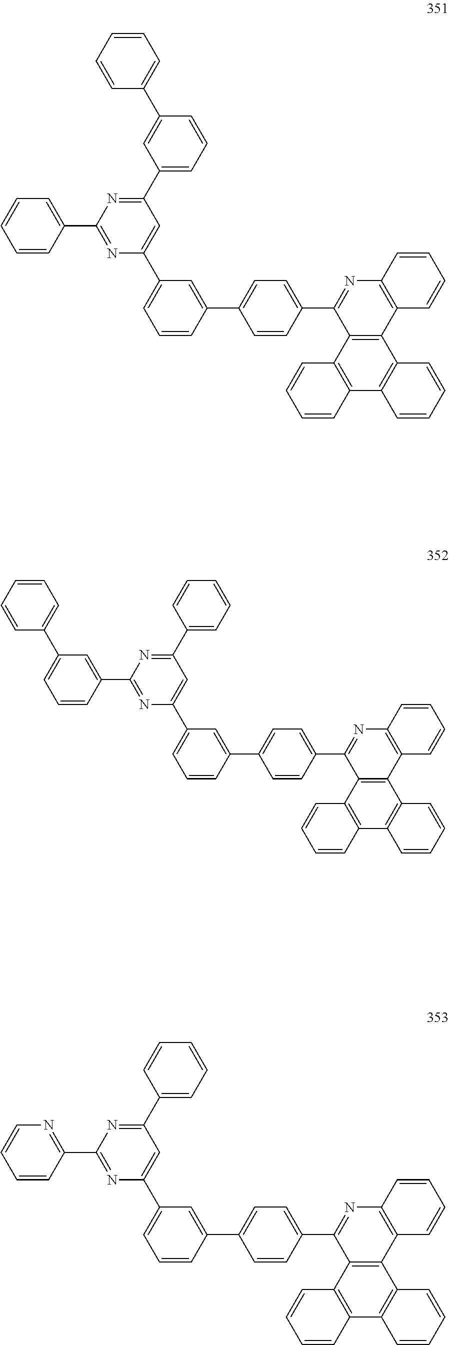 [SMILES: c1ccc(-c2cccc(-c3cc(-c4cccc(-c5ccc(-c6nc7ccccc7c7c8ccccc8c8ccccc8c67)cc5)c4)nc(-c4ccccc4)n3)c2)cc1 ; c1ccc(-c2cccc(-c3nc(-c4ccccc4)cc(-c4cccc(-c5ccc(-c6nc7ccccc7c7c8ccccc8c8ccccc8c67)cc5)c4)n3)c2)cc1 ; c1ccc(-c2cc(-c3cccc(-c4ccc(-c5nc6ccccc6c6c7ccccc7c7ccccc7c56)cc4)c3)nc(-c3ccccn3)n2)cc1]

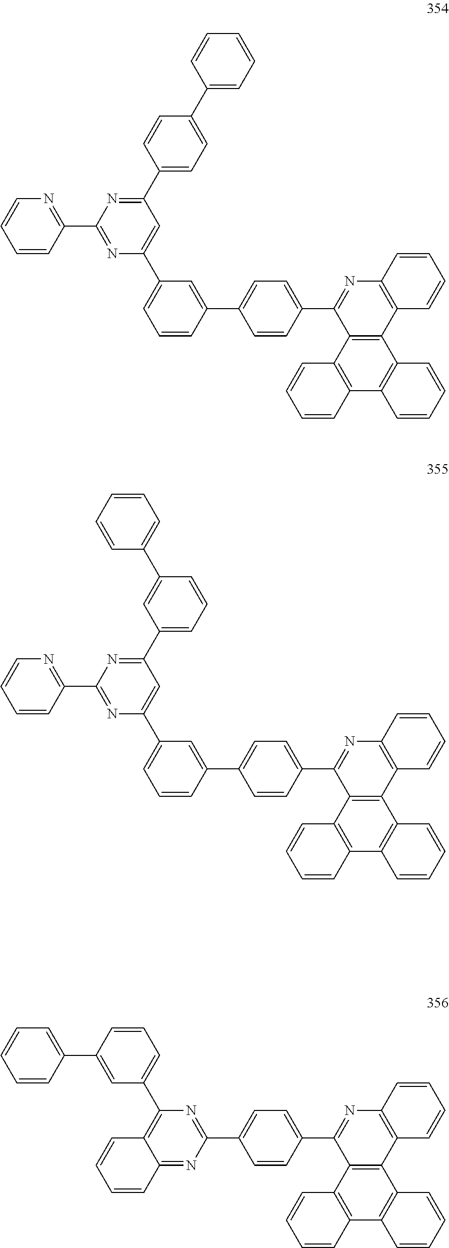 [SMILES: c1ccc(-c2ccc(-c3cc(-c4cccc(-c5ccc(-c6nc7ccccc7c7c8ccccc8c8ccccc8c67)cc5)c4)nc(-c4ccccn4)n3)cc2)cc1 ; c1ccc(-c2cccc(-c3cc(-c4cccc(-c5ccc(-c6nc7ccccc7c7c8ccccc8c8ccccc8c67)cc5)c4)nc(-c4ccccn4)n3)c2)cc1 ; c1ccc(-c2cccc(-c3nc(-c4ccc(-c5nc6ccccc6c6c7ccccc7c7ccccc7c56)cc4)nc4ccccc34)c2)cc1]

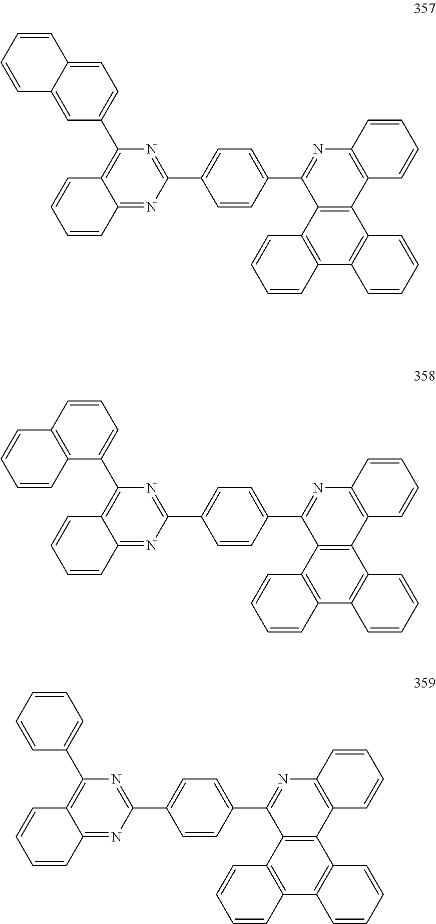 [SMILES: c1ccc2cc(-c3nc(-c4ccc(-c5nc6ccccc6c6c7ccccc7c7ccccc7c56)cc4)nc4ccccc34)ccc2c1 ; c1ccc2c(-c3nc(-c4ccc(-c5nc6ccccc6c6c7ccccc7c7ccccc7c56)cc4)nc4ccccc34)cccc2c1 ; c1ccc(-c2nc(-c3ccc(-c4nc5ccccc5c5c6ccccc6c6ccccc6c45)cc3)nc3ccccc23)cc1]

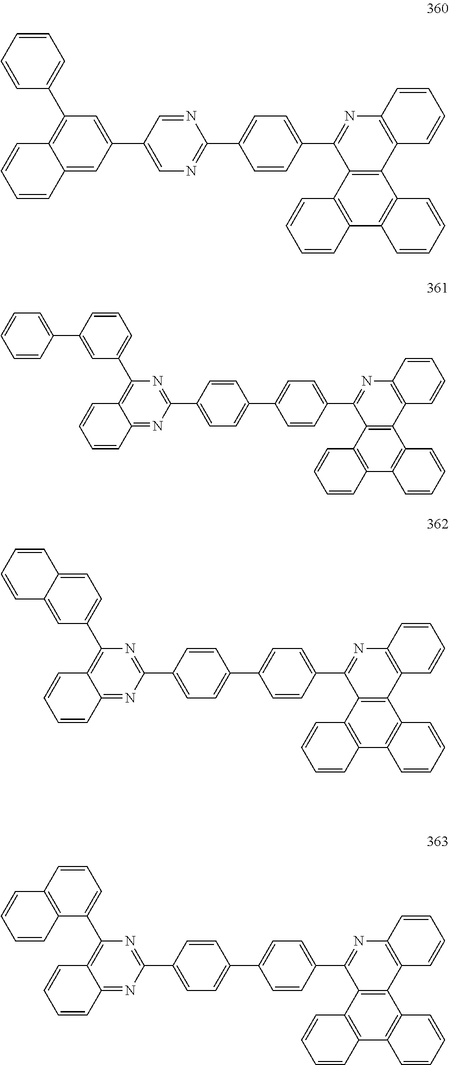 [SMILES: c1ccc(-c2cc(-c3cnc(-c4ccc(-c5nc6ccccc6c6c7ccccc7c7ccccc7c56)cc4)nc3)cc3ccccc23)cc1 ; c1ccc(-c2cccc(-c3nc(-c4ccc(-c5ccc(-c6nc7ccccc7c7c8ccccc8c8ccccc8c67)cc5)cc4)nc4ccccc34)c2)cc1 ; c1ccc2cc(-c3nc(-c4ccc(-c5ccc(-c6nc7ccccc7c7c8ccccc8c8ccccc8c67)cc5)cc4)nc4ccccc34)ccc2c1 ; c1ccc2c(-c3nc(-c4ccc(-c5ccc(-c6nc7ccccc7c7c8ccccc8c8ccccc8c67)cc5)cc4)nc4ccccc34)cccc2c1]

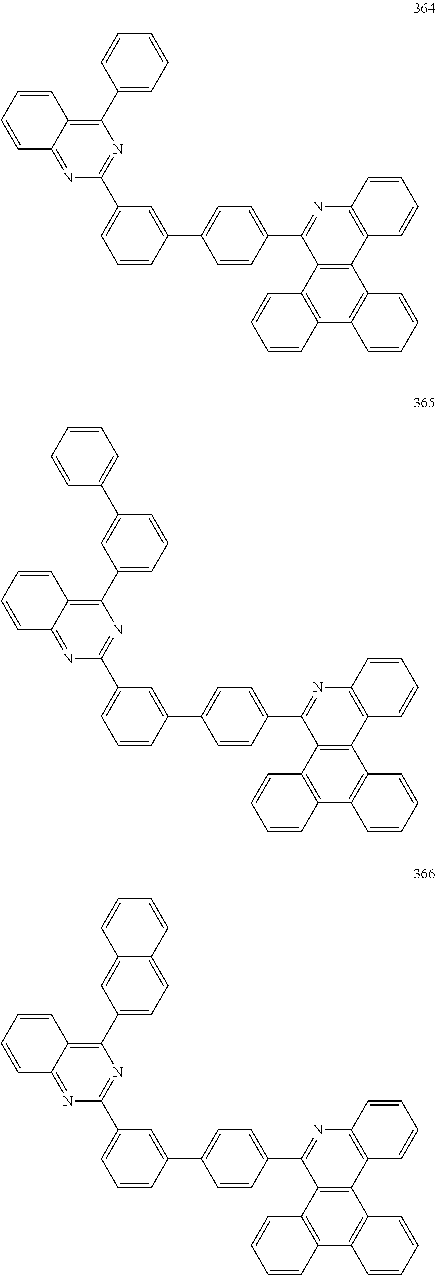 [SMILES: c1ccc(-c2nc(-c3cccc(-c4ccc(-c5nc6ccccc6c6c7ccccc7c7ccccc7c56)cc4)c3)nc3ccccc23)cc1 ; c1ccc(-c2cccc(-c3nc(-c4cccc(-c5ccc(-c6nc7ccccc7c7c8ccccc8c8ccccc8c67)cc5)c4)nc4ccccc34)c2)cc1 ; c1cc(-c2ccc(-c3nc4ccccc4c4c5ccccc5c5ccccc5c34)cc2)cc(-c2nc(-c3ccc4ccccc4c3)c3ccccc3n2)c1]

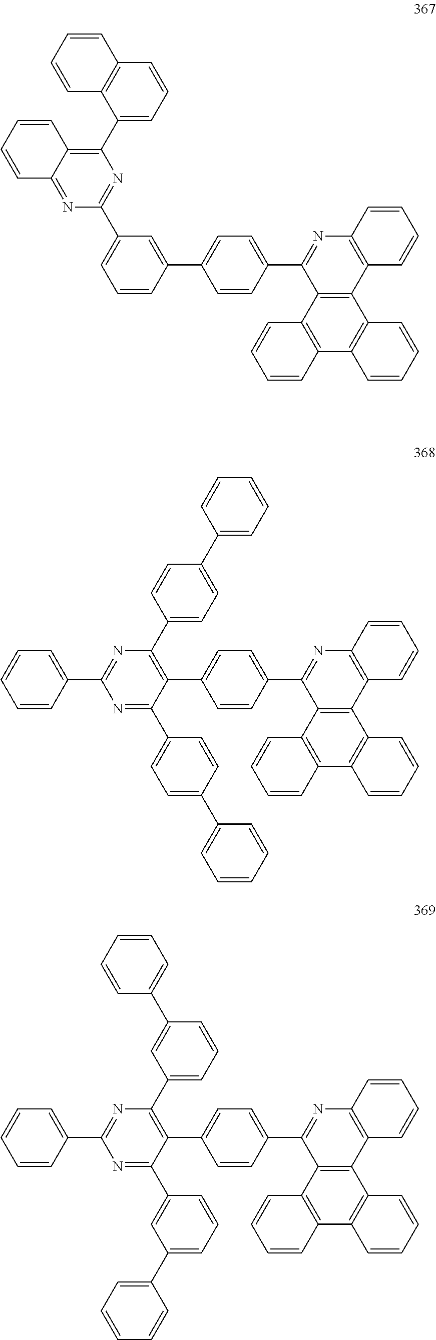 [SMILES: c1cc(-c2ccc(-c3nc4ccccc4c4c5ccccc5c5ccccc5c34)cc2)cc(-c2nc(-c3cccc4ccccc34)c3ccccc3n2)c1 ; c1ccc(-c2ccc(-c3nc(-c4ccccc4)nc(-c4ccc(-c5ccccc5)cc4)c3-c3ccc(-c4nc5ccccc5c5c6ccccc6c6ccccc6c45)cc3)cc2)cc1 ; c1ccc(-c2cccc(-c3nc(-c4ccccc4)nc(-c4cccc(-c5ccccc5)c4)c3-c3ccc(-c4nc5ccccc5c5c6ccccc6c6ccccc6c45)cc3)c2)cc1]

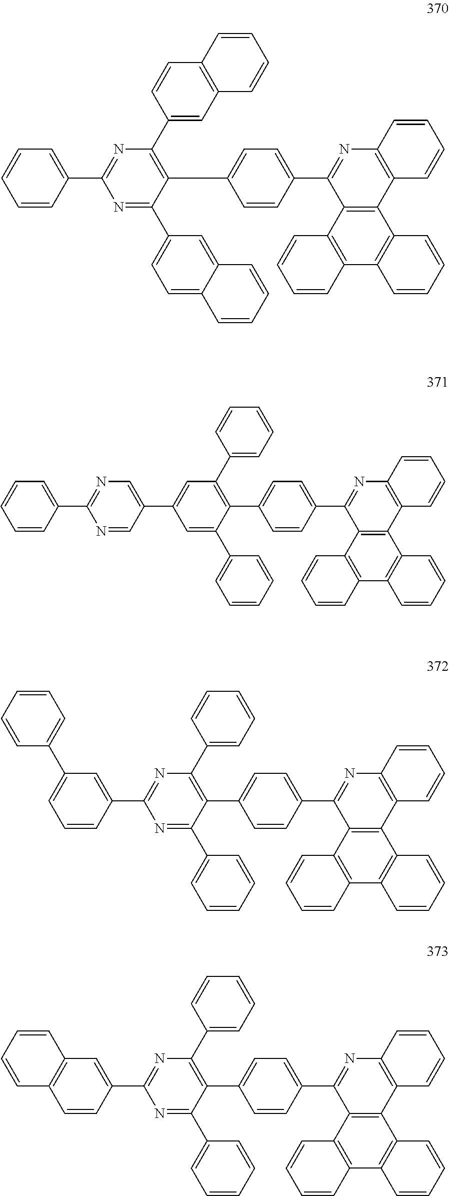 [SMILES: c1ccc(-c2nc(-c3ccc4ccccc4c3)c(-c3ccc(-c4nc5ccccc5c5c6ccccc6c6ccccc6c45)cc3)c(-c3ccc4ccccc4c3)n2)cc1 ; c1ccc(-c2ncc(-c3cc(-c4ccccc4)c(-c4ccc(-c5nc6ccccc6c6c7ccccc7c7ccccc7c56)cc4)c(-c4ccccc4)c3)cn2)cc1 ; c1ccc(-c2cccc(-c3nc(-c4ccccc4)c(-c4ccc(-c5nc6ccccc6c6c7ccccc7c7ccccc7c56)cc4)c(-c4ccccc4)n3)c2)cc1 ; c1ccc(-c2nc(-c3ccc4ccccc4c3)nc(-c3ccccc3)c2-c2ccc(-c3nc4ccccc4c4c5ccccc5c5ccccc5c34)cc2)cc1]

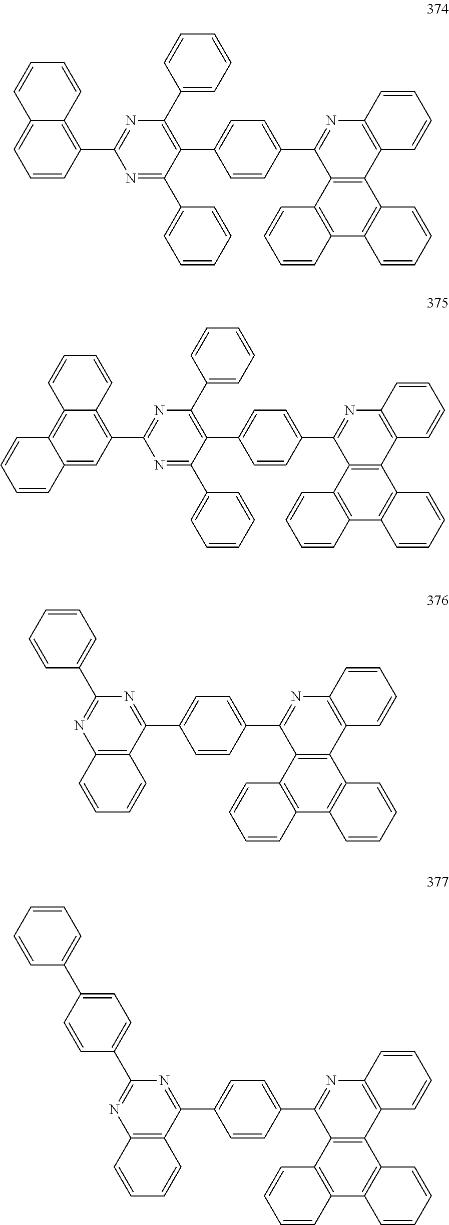 [SMILES: c1ccc(-c2nc(-c3cccc4ccccc34)nc(-c3ccccc3)c2-c2ccc(-c3nc4ccccc4c4c5ccccc5c5ccccc5c34)cc2)cc1 ; c1ccc(-c2nc(-c3cc4ccccc4c4ccccc34)nc(-c3ccccc3)c2-c2ccc(-c3nc4ccccc4c4c5ccccc5c5ccccc5c34)cc2)cc1 ; c1ccc(-c2nc(-c3ccc(-c4nc5ccccc5c5c6ccccc6c6ccccc6c45)cc3)c3ccccc3n2)cc1 ; c1ccc(-c2ccc(-c3nc(-c4ccc(-c5nc6ccccc6c6c7ccccc7c7ccccc7c56)cc4)c4ccccc4n3)cc2)cc1]

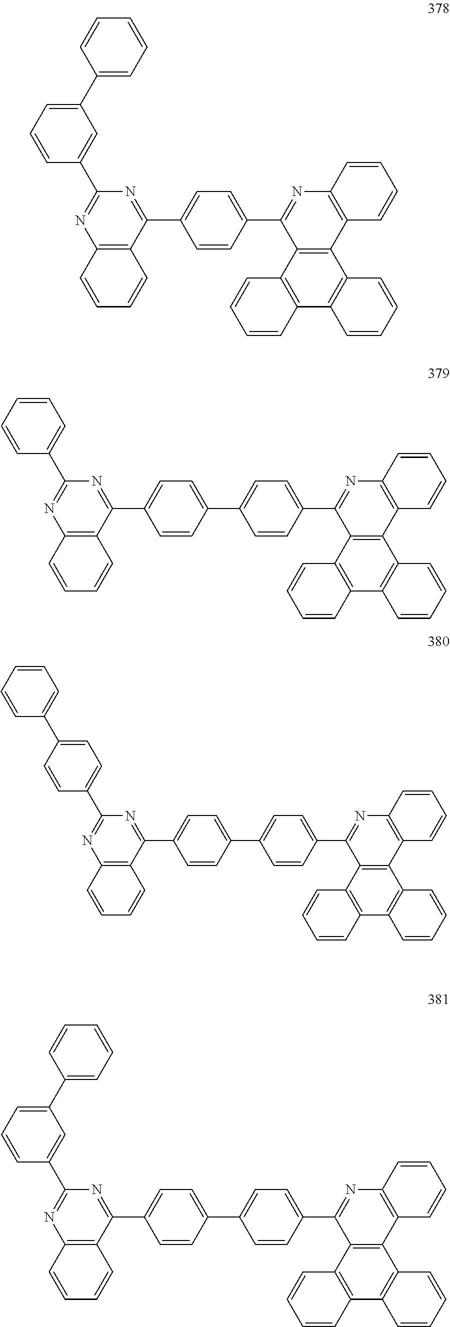 [SMILES: c1ccc(-c2cccc(-c3nc(-c4ccc(-c5nc6ccccc6c6c7ccccc7c7ccccc7c56)cc4)c4ccccc4n3)c2)cc1 ; c1ccc(-c2nc(-c3ccc(-c4ccc(-c5nc6ccccc6c6c7ccccc7c7ccccc7c56)cc4)cc3)c3ccccc3n2)cc1 ; c1ccc(-c2ccc(-c3nc(-c4ccc(-c5ccc(-c6nc7ccccc7c7c8ccccc8c8ccccc8c67)cc5)cc4)c4ccccc4n3)cc2)cc1 ; c1ccc(-c2cccc(-c3nc(-c4ccc(-c5ccc(-c6nc7ccccc7c7c8ccccc8c8ccccc8c67)cc5)cc4)c4ccccc4n3)c2)cc1]

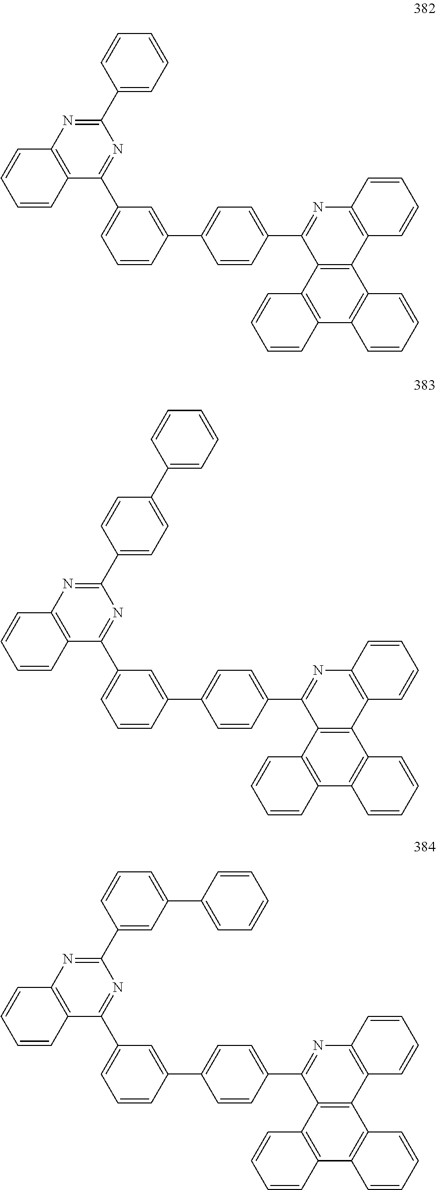 [SMILES: c1ccc(-c2nc(-c3cccc(-c4ccc(-c5nc6ccccc6c6c7ccccc7c7ccccc7c56)cc4)c3)c3ccccc3n2)cc1 ; c1ccc(-c2ccc(-c3nc(-c4cccc(-c5ccc(-c6nc7ccccc7c7c8ccccc8c8ccccc8c67)cc5)c4)c4ccccc4n3)cc2)cc1 ; c1ccc(-c2cccc(-c3nc(-c4cccc(-c5ccc(-c6nc7ccccc7c7c8ccccc8c8ccccc8c67)cc5)c4)c4ccccc4n3)c2)cc1]

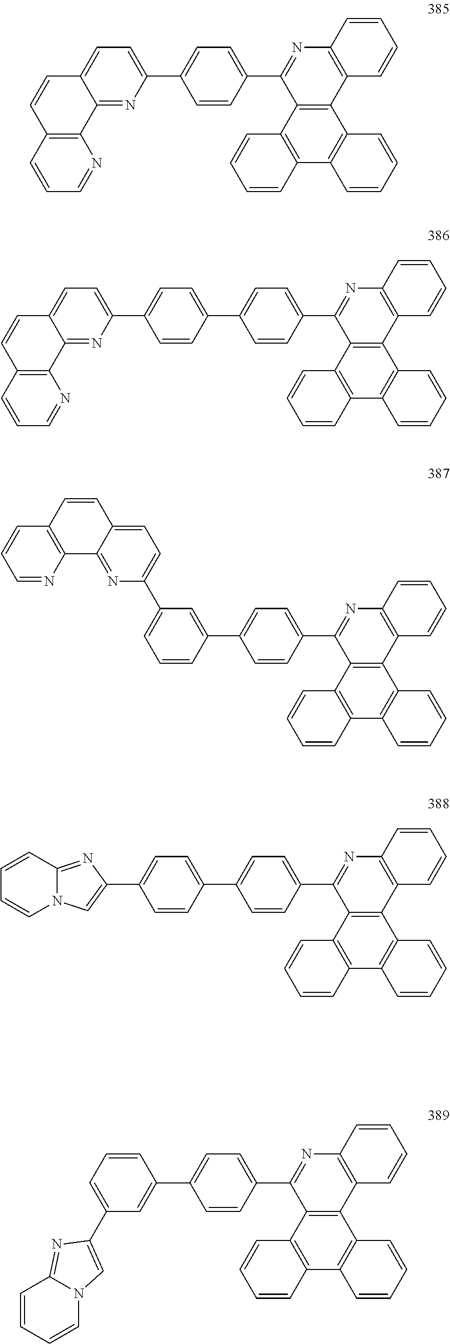 [SMILES: c1cnc2c(c1)ccc1ccc(-c3ccc(-c4nc5ccccc5c5c6ccccc6c6ccccc6c45)cc3)nc12 ; c1cnc2c(c1)ccc1ccc(-c3ccc(-c4ccc(-c5nc6ccccc6c6c7ccccc7c7ccccc7c56)cc4)cc3)nc12 ; c1cc(-c2ccc(-c3nc4ccccc4c4c5ccccc5c5ccccc5c34)cc2)cc(-c2ccc3ccc4cccnc4c3n2)c1 ; c1ccc2c(c1)nc(-c1ccc(-c3ccc(-c4cn5ccccc5n4)cc3)cc1)c1c3ccccc3c3ccccc3c21 ; c1cc(-c2ccc(-c3nc4ccccc4c4c5ccccc5c5ccccc5c34)cc2)cc(-c2cn3ccccc3n2)c1]

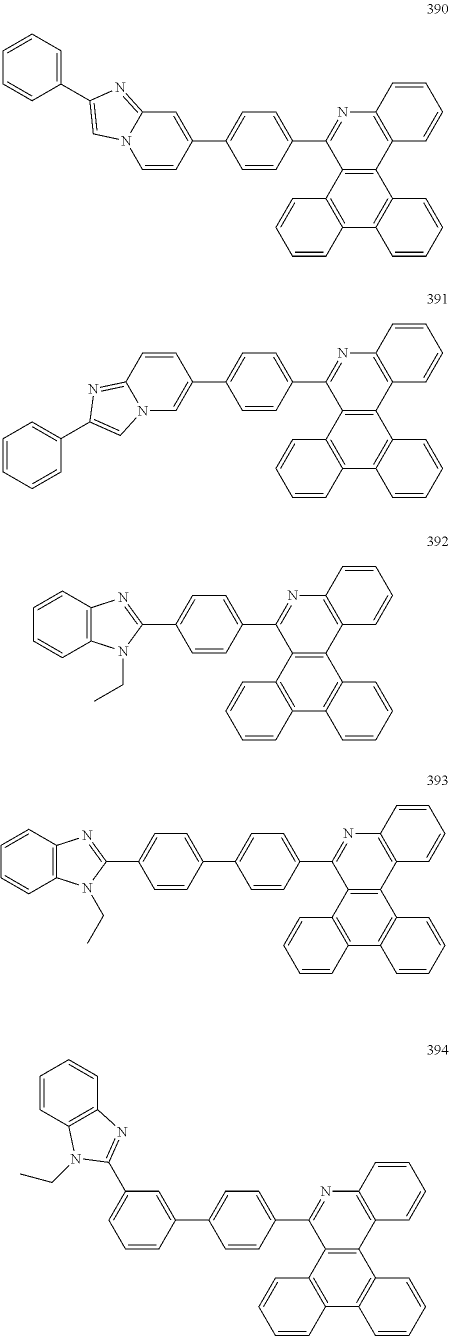 [SMILES: c1ccc(-c2cn3ccc(-c4ccc(-c5nc6ccccc6c6c7ccccc7c7ccccc7c56)cc4)cc3n2)cc1 ; c1ccc(-c2cn3cc(-c4ccc(-c5nc6ccccc6c6c7ccccc7c7ccccc7c56)cc4)ccc3n2)cc1 ; CCn1c(-c2ccc(-c3nc4ccccc4c4c5ccccc5c5ccccc5c34)cc2)nc2ccccc21 ; CCn1c(-c2ccc(-c3ccc(-c4nc5ccccc5c5c6ccccc6c6ccccc6c45)cc3)cc2)nc2ccccc21 ; CCn1c(-c2cccc(-c3ccc(-c4nc5ccccc5c5c6ccccc6c6ccccc6c45)cc3)c2)nc2ccccc21]

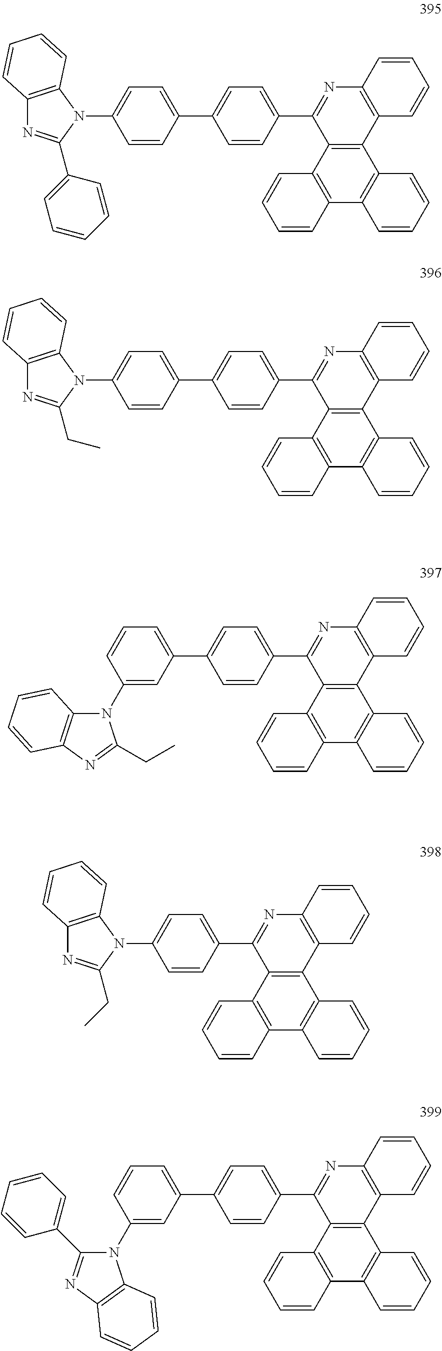 [SMILES: c1ccc(-c2nc3ccccc3n2-c2ccc(-c3ccc(-c4nc5ccccc5c5c6ccccc6c6ccccc6c45)cc3)cc2)cc1 ; CCc1nc2ccccc2n1-c1ccc(-c2ccc(-c3nc4ccccc4c4c5ccccc5c5ccccc5c34)cc2)cc1 ; CCc1nc2ccccc2n1-c1cccc(-c2ccc(-c3nc4ccccc4c4c5ccccc5c5ccccc5c34)cc2)c1 ; CCc1nc2ccccc2n1-c1ccc(-c2nc3ccccc3c3c4ccccc4c4ccccc4c23)cc1 ; c1ccc(-c2nc3ccccc3n2-c2cccc(-c3ccc(-c4nc5ccccc5c5c6ccccc6c6ccccc6c45)cc3)c2)cc1]

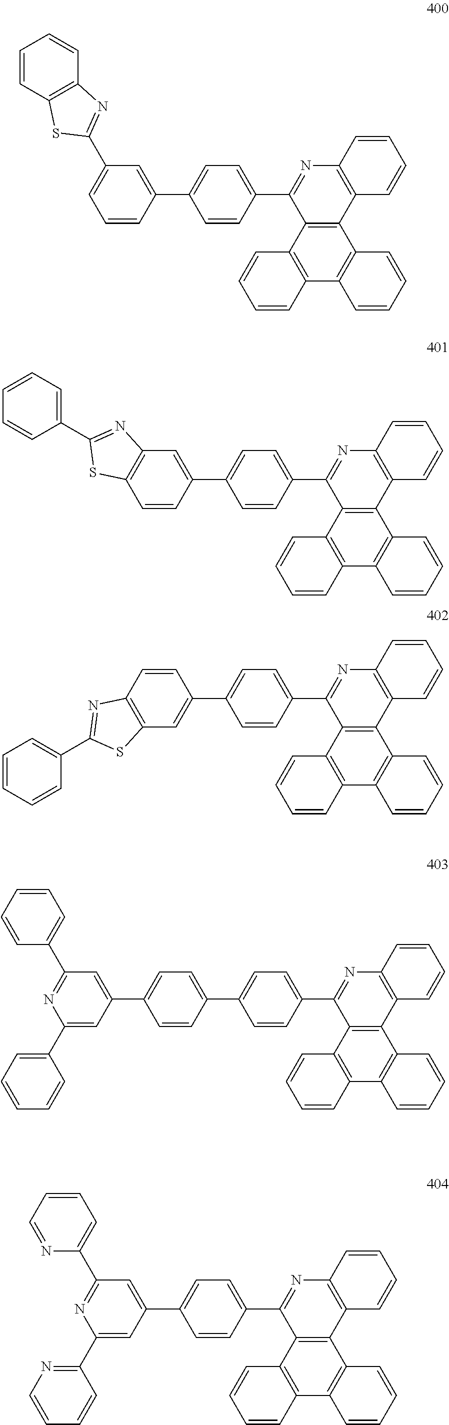

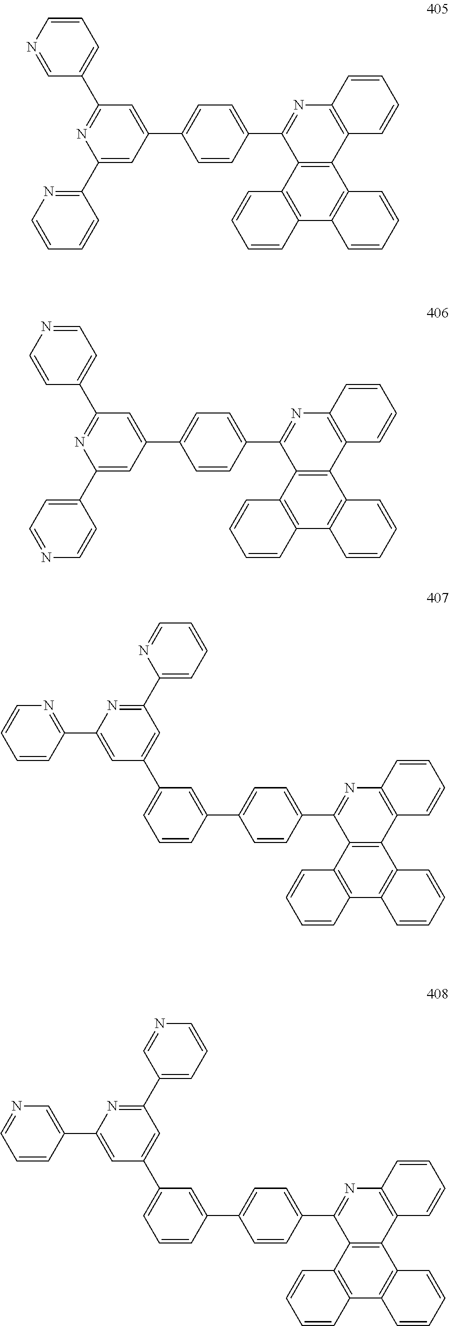 [SMILES: c1ccc(-c2cc(-c3ccc(-c4nc5ccccc5c5c6ccccc6c6ccccc6c45)cc3)cc(-c3cccnc3)n2)nc1 ; c1ccc2c(c1)nc(-c1ccc(-c3cc(-c4ccncc4)nc(-c4ccncc4)c3)cc1)c1c3ccccc3c3ccccc3c21 ; c1ccc(-c2cc(-c3cccc(-c4ccc(-c5nc6ccccc6c6c7ccccc7c7ccccc7c56)cc4)c3)cc(-c3ccccn3)n2)nc1 ; c1cc(-c2ccc(-c3nc4ccccc4c4c5ccccc5c5ccccc5c34)cc2)cc(-c2cc(-c3cccnc3)nc(-c3cccnc3)c2)c1]

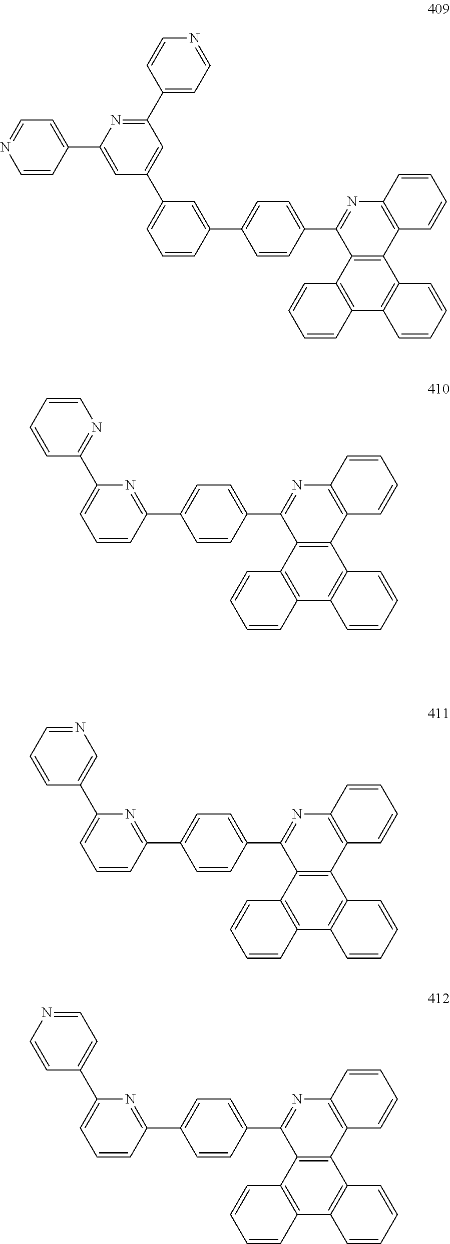 [SMILES: c1cc(-c2ccc(-c3nc4ccccc4c4c5ccccc5c5ccccc5c34)cc2)cc(-c2cc(-c3ccncc3)nc(-c3ccncc3)c2)c1 ; c1ccc(-c2cccc(-c3ccc(-c4nc5ccccc5c5c6ccccc6c6ccccc6c45)cc3)n2)nc1 ; c1cncc(-c2cccc(-c3ccc(-c4nc5ccccc5c5c6ccccc6c6ccccc6c45)cc3)n2)c1 ; c1cc(-c2ccncc2)nc(-c2ccc(-c3nc4ccccc4c4c5ccccc5c5ccccc5c34)cc2)c1]

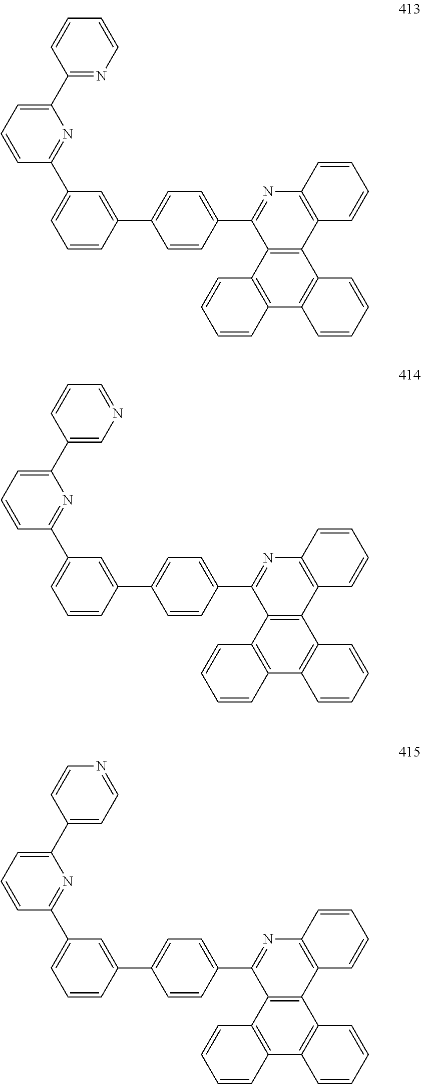 [SMILES: c1ccc(-c2cccc(-c3cccc(-c4ccc(-c5nc6ccccc6c6c7ccccc7c7ccccc7c56)cc4)c3)n2)nc1 ; c1cncc(-c2cccc(-c3cccc(-c4ccc(-c5nc6ccccc6c6c7ccccc7c7ccccc7c56)cc4)c3)n2)c1 ; c1cc(-c2ccc(-c3nc4ccccc4c4c5ccccc5c5ccccc5c34)cc2)cc(-c2cccc(-c3ccncc3)n2)c1]

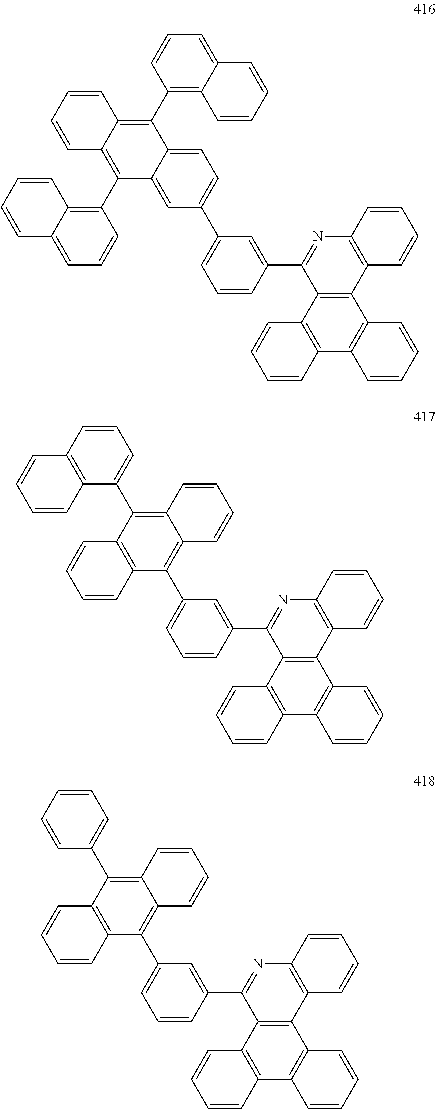 [SMILES: c1cc(-c2ccc3c(-c4cccc5ccccc45)c4ccccc4c(-c4cccc5ccccc45)c3c2)cc(-c2nc3ccccc3c3c4ccccc4c4ccccc4c23)c1 ; c1cc(-c2c3ccccc3c(-c3cccc4ccccc34)c3ccccc23)cc(-c2nc3ccccc3c3c4ccccc4c4ccccc4c23)c1 ; c1ccc(-c2c3ccccc3c(-c3cccc(-c4nc5ccccc5c5c6ccccc6c6ccccc6c45)c3)c3ccccc23)cc1]

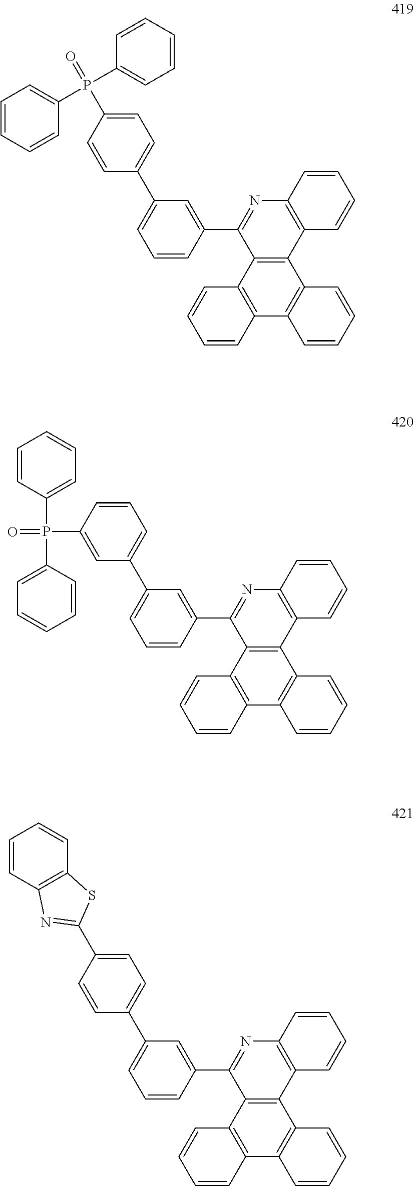 [SMILES: O=P(c1ccccc1)(c1ccccc1)c1ccc(-c2cccc(-c3nc4ccccc4c4c5ccccc5c5ccccc5c34)c2)cc1 ; O=P(c1ccccc1)(c1ccccc1)c1cccc(-c2cccc(-c3nc4ccccc4c4c5ccccc5c5ccccc5c34)c2)c1 ; c1cc(-c2ccc(-c3nc4ccccc4s3)cc2)cc(-c2nc3ccccc3c3c4ccccc4c4ccccc4c23)c1]

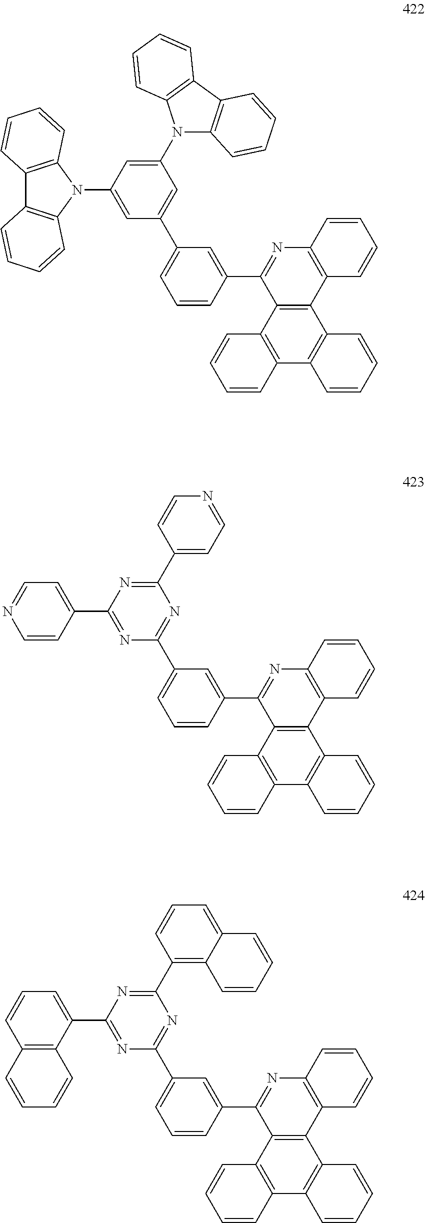 [SMILES: c1cc(-c2cc(-n3c4ccccc4c4ccccc43)cc(-n3c4ccccc4c4ccccc43)c2)cc(-c2nc3ccccc3c3c4ccccc4c4ccccc4c23)c1 ; c1cc(-c2nc(-c3ccncc3)nc(-c3ccncc3)n2)cc(-c2nc3ccccc3c3c4ccccc4c4ccccc4c23)c1 ; c1cc(-c2nc(-c3cccc4ccccc34)nc(-c3cccc4ccccc34)n2)cc(-c2nc3ccccc3c3c4ccccc4c4ccccc4c23)c1]

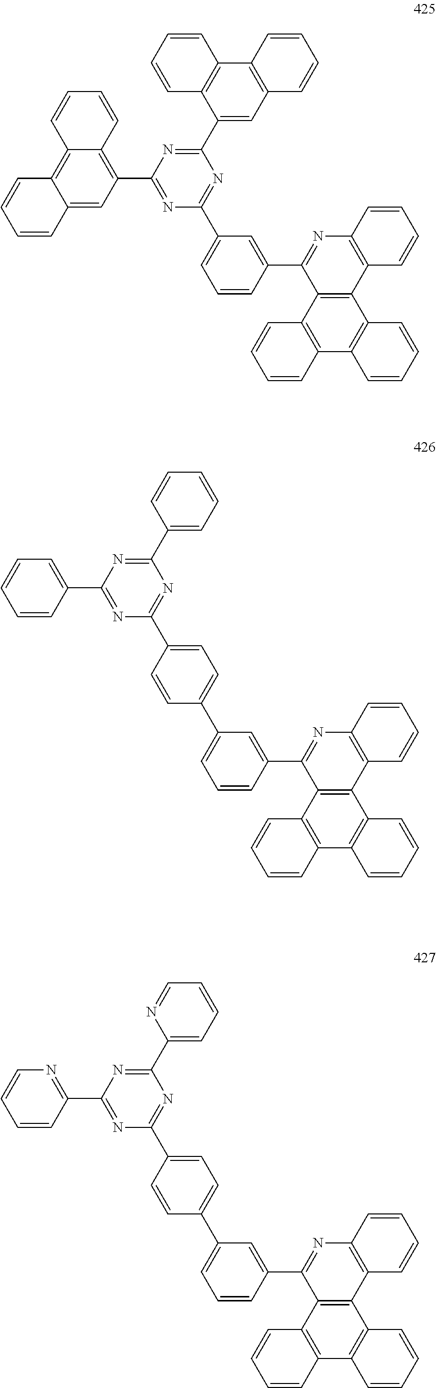 [SMILES: c1cc(-c2nc(-c3cc4ccccc4c4ccccc34)nc(-c3cc4ccccc4c4ccccc34)n2)cc(-c2nc3ccccc3c3c4ccccc4c4ccccc4c23)c1 ; c1ccc(-c2nc(-c3ccccc3)nc(-c3ccc(-c4cccc(-c5nc6ccccc6c6c7ccccc7c7ccccc7c56)c4)cc3)n2)cc1 ; c1ccc(-c2nc(-c3ccc(-c4cccc(-c5nc6ccccc6c6c7ccccc7c7ccccc7c56)c4)cc3)nc(-c3ccccn3)n2)nc1]

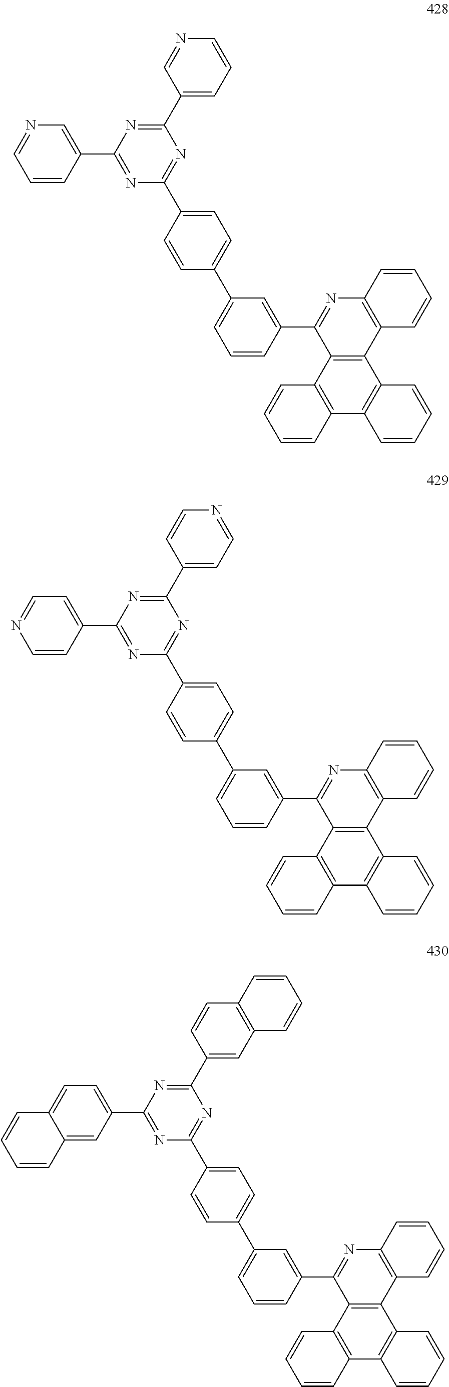 [SMILES: c1cncc(-c2nc(-c3ccc(-c4cccc(-c5nc6ccccc6c6c7ccccc7c7ccccc7c56)c4)cc3)nc(-c3cccnc3)n2)c1 ; c1cc(-c2ccc(-c3nc(-c4ccncc4)nc(-c4ccncc4)n3)cc2)cc(-c2nc3ccccc3c3c4ccccc4c4ccccc4c23)c1 ; c1cc(-c2ccc(-c3nc(-c4ccc5ccccc5c4)nc(-c4ccc5ccccc5c4)n3)cc2)cc(-c2nc3ccccc3c3c4ccccc4c4ccccc4c23)c1]

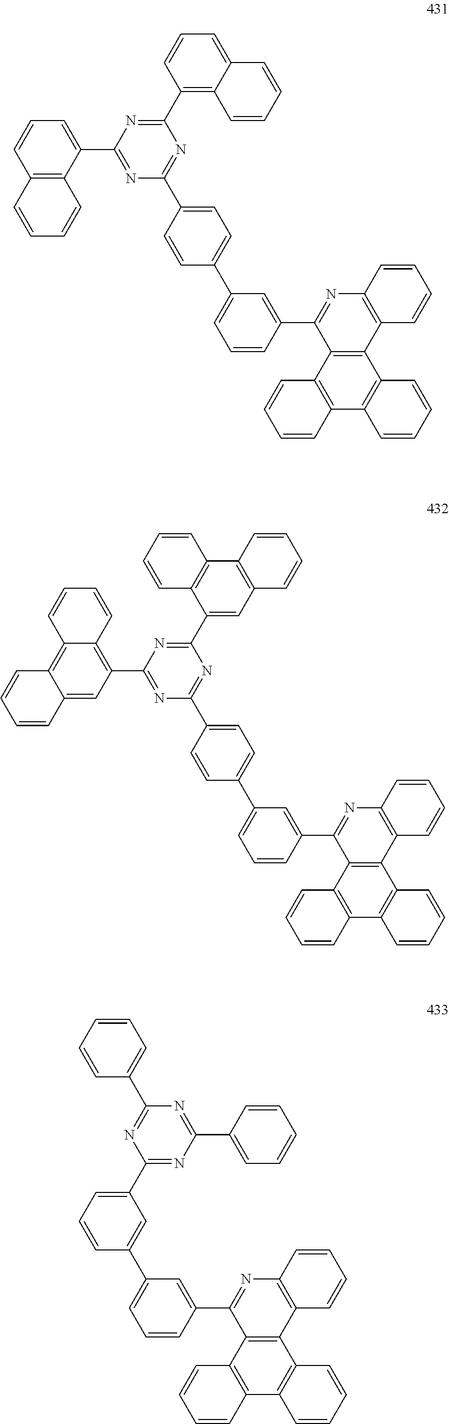 [SMILES: c1cc(-c2ccc(-c3nc(-c4cccc5ccccc45)nc(-c4cccc5ccccc45)n3)cc2)cc(-c2nc3ccccc3c3c4ccccc4c4ccccc4c23)c1 ; c1cc(-c2ccc(-c3nc(-c4cc5ccccc5c5ccccc45)nc(-c4cc5ccccc5c5ccccc45)n3)cc2)cc(-c2nc3ccccc3c3c4ccccc4c4ccccc4c23)c1 ; c1ccc(-c2nc(-c3ccccc3)nc(-c3cccc(-c4cccc(-c5nc6ccccc6c6c7ccccc7c7ccccc7c56)c4)c3)n2)cc1]

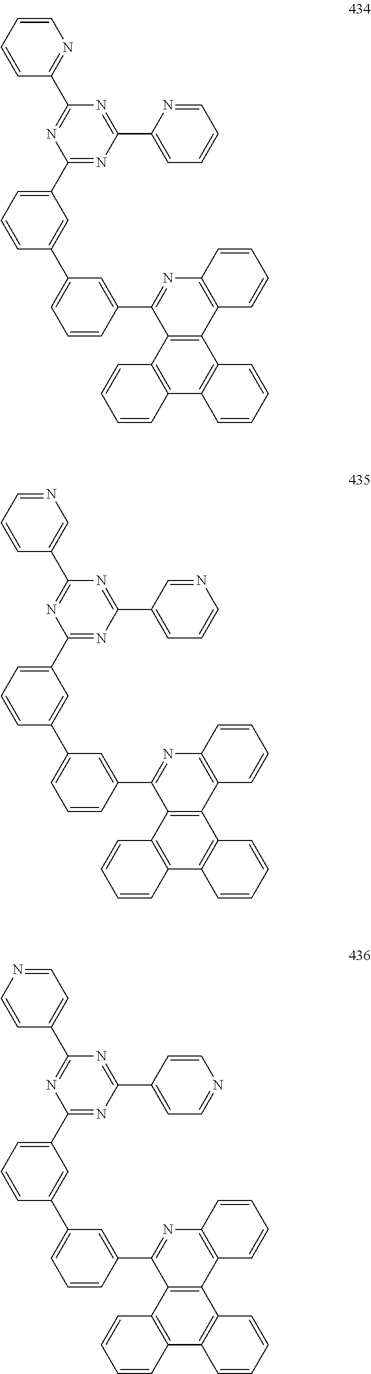 [SMILES: c1ccc(-c2nc(-c3cccc(-c4cccc(-c5nc6ccccc6c6c7ccccc7c7ccccc7c56)c4)c3)nc(-c3ccccn3)n2)nc1 ; c1cncc(-c2nc(-c3cccnc3)nc(-c3cccc(-c4cccc(-c5nc6ccccc6c6c7ccccc7c7ccccc7c56)c4)c3)n2)c1 ; c1cc(-c2cccc(-c3nc4ccccc4c4c5ccccc5c5ccccc5c34)c2)cc(-c2nc(-c3ccncc3)nc(-c3ccncc3)n2)c1]

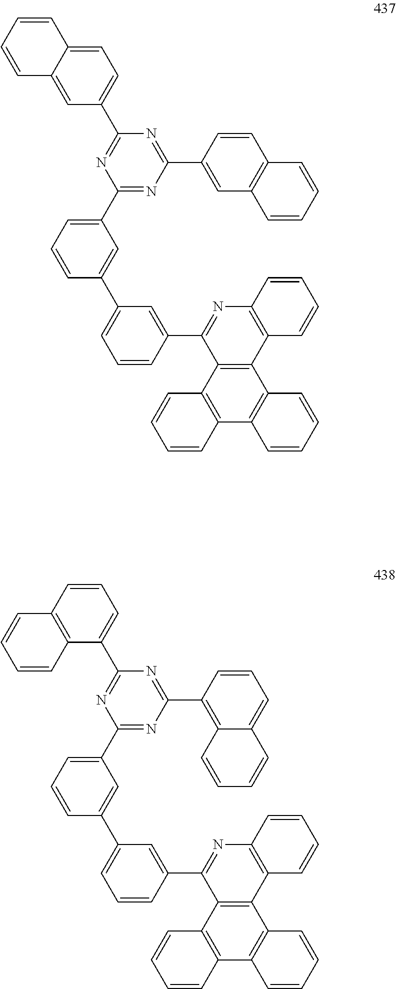 [SMILES: c1cc(-c2cccc(-c3nc4ccccc4c4c5ccccc5c5ccccc5c34)c2)cc(-c2nc(-c3ccc4ccccc4c3)nc(-c3ccc4ccccc4c3)n2)c1 ; c1cc(-c2cccc(-c3nc4ccccc4c4c5ccccc5c5ccccc5c34)c2)cc(-c2nc(-c3cccc4ccccc34)nc(-c3cccc4ccccc34)n2)c1]

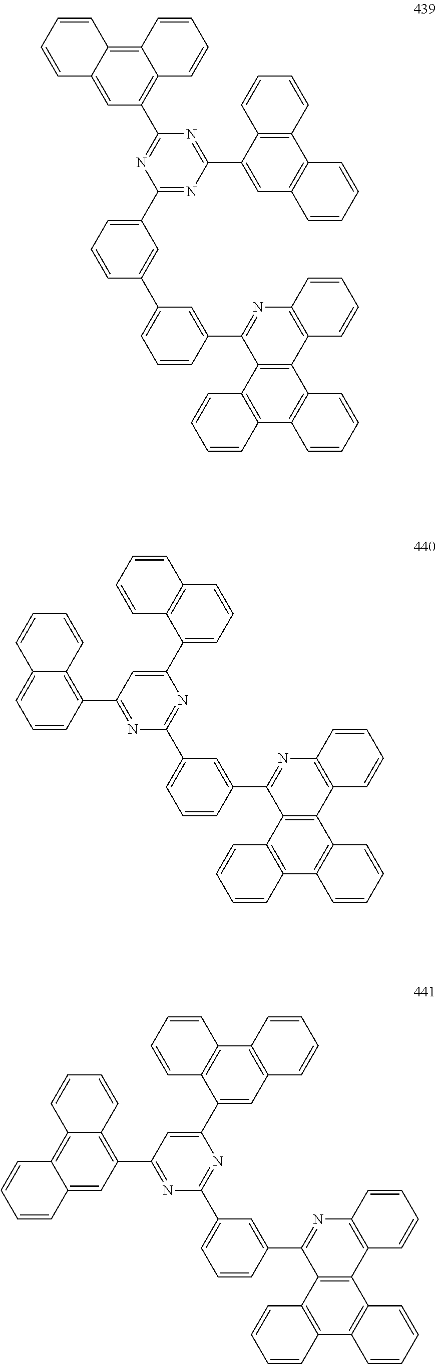 [SMILES: c1cc(-c2cccc(-c3nc4ccccc4c4c5ccccc5c5ccccc5c34)c2)cc(-c2nc(-c3cc4ccccc4c4ccccc34)nc(-c3cc4ccccc4c4ccccc34)n2)c1 ; c1cc(-c2nc(-c3cccc4ccccc34)cc(-c3cccc4ccccc34)n2)cc(-c2nc3ccccc3c3c4ccccc4c4ccccc4c23)c1 ; c1cc(-c2nc(-c3cc4ccccc4c4ccccc34)cc(-c3cc4ccccc4c4ccccc34)n2)cc(-c2nc3ccccc3c3c4ccccc4c4ccccc4c23)c1]

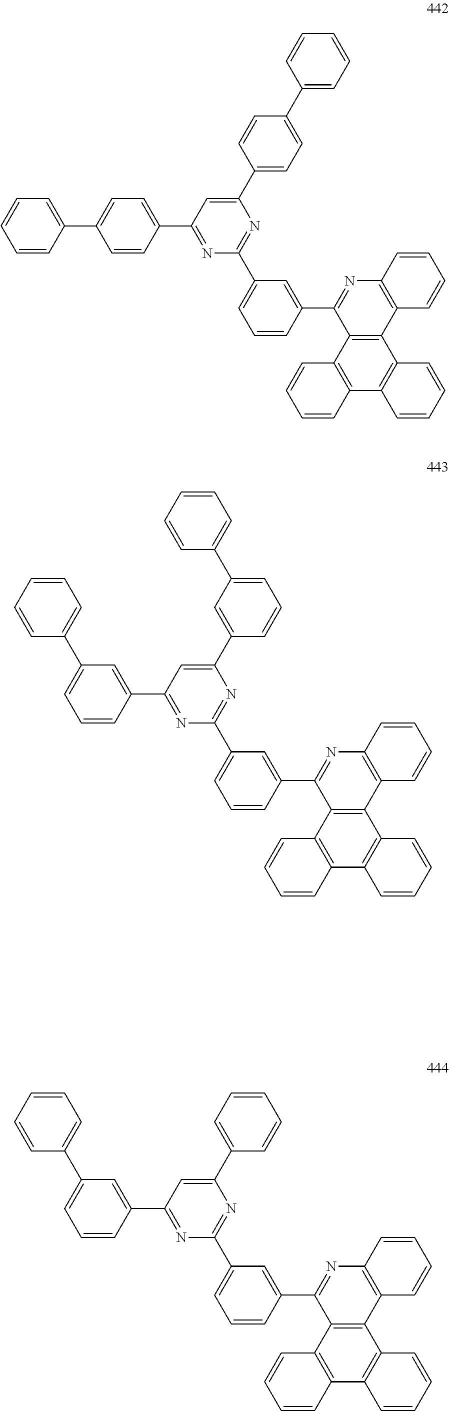 [SMILES: c1ccc(-c2ccc(-c3cc(-c4ccc(-c5ccccc5)cc4)nc(-c4cccc(-c5nc6ccccc6c6c7ccccc7c7ccccc7c56)c4)n3)cc2)cc1 ; c1ccc(-c2cccc(-c3cc(-c4cccc(-c5ccccc5)c4)nc(-c4cccc(-c5nc6ccccc6c6c7ccccc7c7ccccc7c56)c4)n3)c2)cc1 ; c1ccc(-c2cccc(-c3cc(-c4ccccc4)nc(-c4cccc(-c5nc6ccccc6c6c7ccccc7c7ccccc7c56)c4)n3)c2)cc1]

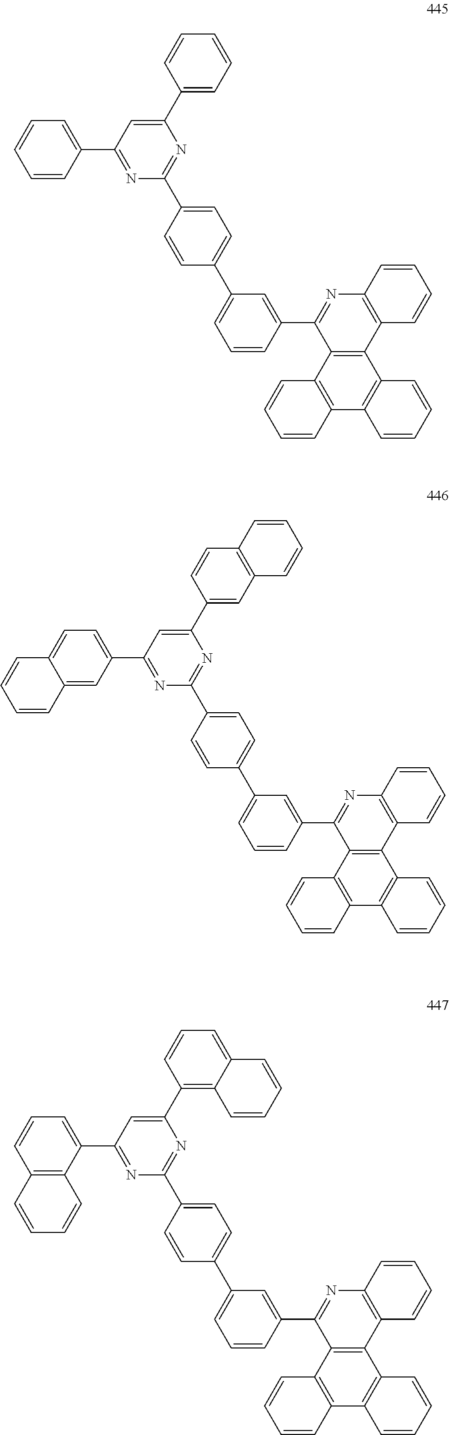 [SMILES: c1ccc(-c2cc(-c3ccccc3)nc(-c3ccc(-c4cccc(-c5nc6ccccc6c6c7ccccc7c7ccccc7c56)c4)cc3)n2)cc1 ; c1cc(-c2ccc(-c3nc(-c4ccc5ccccc5c4)cc(-c4ccc5ccccc5c4)n3)cc2)cc(-c2nc3ccccc3c3c4ccccc4c4ccccc4c23)c1 ; c1cc(-c2ccc(-c3nc(-c4cccc5ccccc45)cc(-c4cccc5ccccc45)n3)cc2)cc(-c2nc3ccccc3c3c4ccccc4c4ccccc4c23)c1]

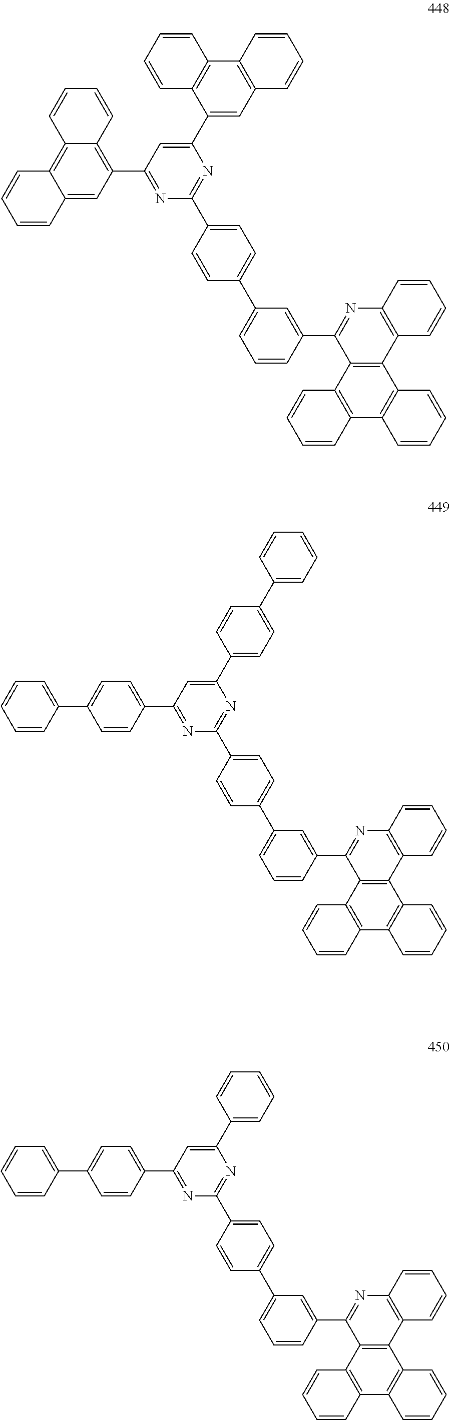 [SMILES: c1cc(-c2ccc(-c3nc(-c4cc5ccccc5c5ccccc45)cc(-c4cc5ccccc5c5ccccc45)n3)cc2)cc(-c2nc3ccccc3c3c4ccccc4c4ccccc4c23)c1 ; c1ccc(-c2ccc(-c3cc(-c4ccc(-c5ccccc5)cc4)nc(-c4ccc(-c5cccc(-c6nc7ccccc7c7c8ccccc8c8ccccc8c67)c5)cc4)n3)cc2)cc1 ; c1ccc(-c2ccc(-c3cc(-c4ccccc4)nc(-c4ccc(-c5cccc(-c6nc7ccccc7c7c8ccccc8c8ccccc8c67)c5)cc4)n3)cc2)cc1]

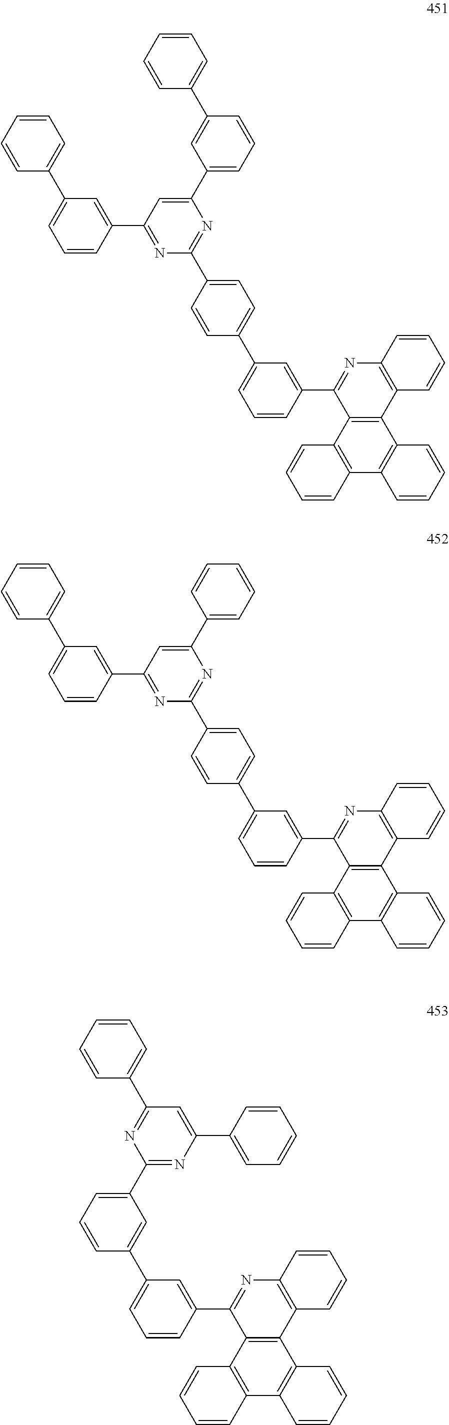 [SMILES: c1ccc(-c2cccc(-c3cc(-c4cccc(-c5ccccc5)c4)nc(-c4ccc(-c5cccc(-c6nc7ccccc7c7c8ccccc8c8ccccc8c67)c5)cc4)n3)c2)cc1 ; c1ccc(-c2cccc(-c3cc(-c4ccccc4)nc(-c4ccc(-c5cccc(-c6nc7ccccc7c7c8ccccc8c8ccccc8c67)c5)cc4)n3)c2)cc1 ; c1ccc(-c2cc(-c3ccccc3)nc(-c3cccc(-c4cccc(-c5nc6ccccc6c6c7ccccc7c7ccccc7c56)c4)c3)n2)cc1]

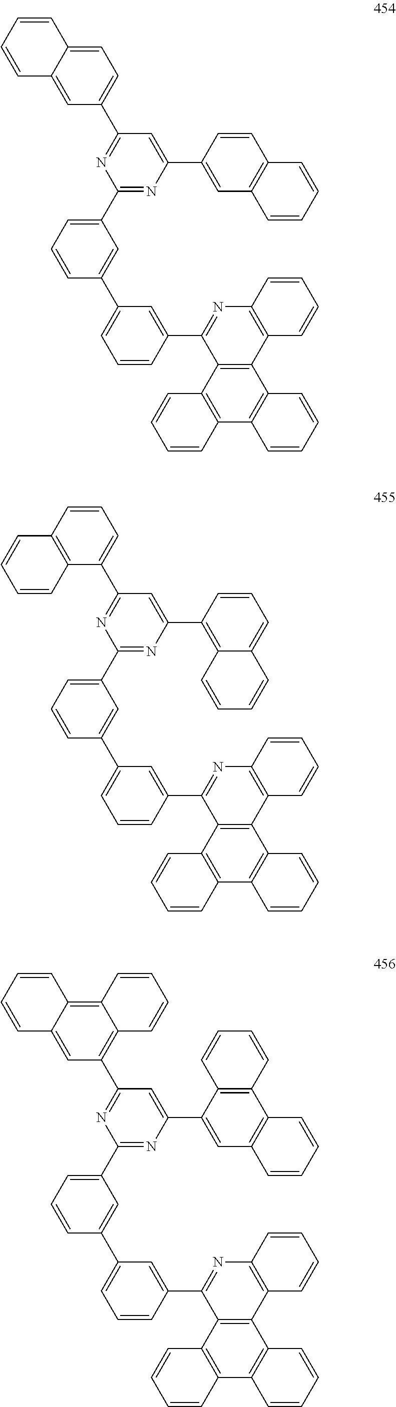 [SMILES: c1cc(-c2cccc(-c3nc4ccccc4c4c5ccccc5c5ccccc5c34)c2)cc(-c2nc(-c3ccc4ccccc4c3)cc(-c3ccc4ccccc4c3)n2)c1 ; c1cc(-c2cccc(-c3nc4ccccc4c4c5ccccc5c5ccccc5c34)c2)cc(-c2nc(-c3cccc4ccccc34)cc(-c3cccc4ccccc34)n2)c1 ; c1cc(-c2cccc(-c3nc4ccccc4c4c5ccccc5c5ccccc5c34)c2)cc(-c2nc(-c3cc4ccccc4c4ccccc34)cc(-c3cc4ccccc4c4ccccc34)n2)c1]

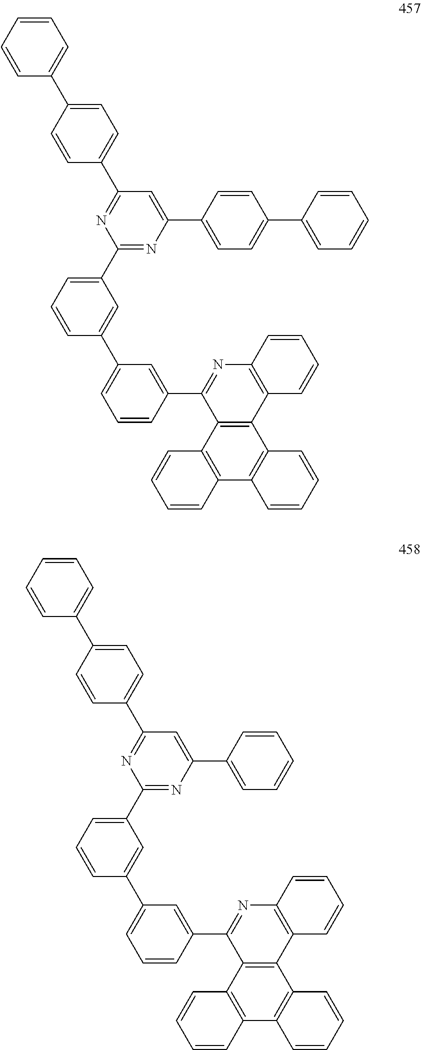 [SMILES: c1ccc(-c2ccc(-c3cc(-c4ccc(-c5ccccc5)cc4)nc(-c4cccc(-c5cccc(-c6nc7ccccc7c7c8ccccc8c8ccccc8c67)c5)c4)n3)cc2)cc1 ; c1ccc(-c2ccc(-c3cc(-c4ccccc4)nc(-c4cccc(-c5cccc(-c6nc7ccccc7c7c8ccccc8c8ccccc8c67)c5)c4)n3)cc2)cc1]

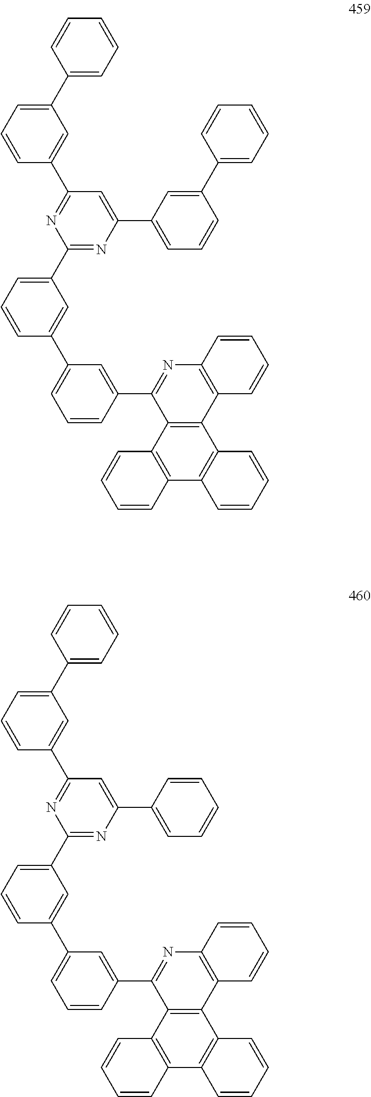 [SMILES: c1ccc(-c2cccc(-c3cc(-c4cccc(-c5ccccc5)c4)nc(-c4cccc(-c5cccc(-c6nc7ccccc7c7c8ccccc8c8ccccc8c67)c5)c4)n3)c2)cc1 ; c1ccc(-c2cccc(-c3cc(-c4ccccc4)nc(-c4cccc(-c5cccc(-c6nc7ccccc7c7c8ccccc8c8ccccc8c67)c5)c4)n3)c2)cc1]

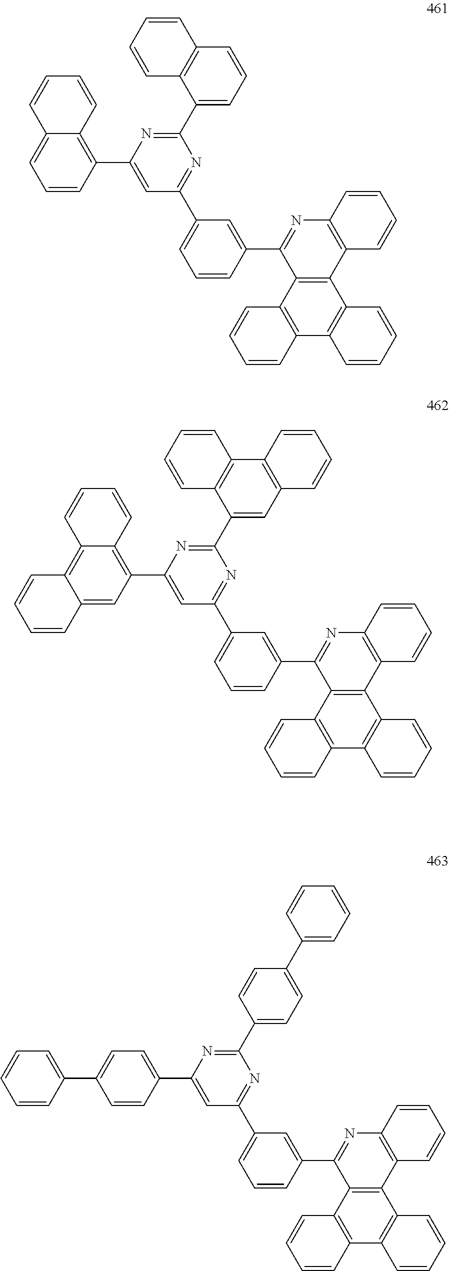 [SMILES: c1cc(-c2cc(-c3cccc4ccccc34)nc(-c3cccc4ccccc34)n2)cc(-c2nc3ccccc3c3c4ccccc4c4ccccc4c23)c1 ; c1cc(-c2cc(-c3cc4ccccc4c4ccccc34)nc(-c3cc4ccccc4c4ccccc34)n2)cc(-c2nc3ccccc3c3c4ccccc4c4ccccc4c23)c1 ; c1ccc(-c2ccc(-c3cc(-c4cccc(-c5nc6ccccc6c6c7ccccc7c7ccccc7c56)c4)nc(-c4ccc(-c5ccccc5)cc4)n3)cc2)cc1]

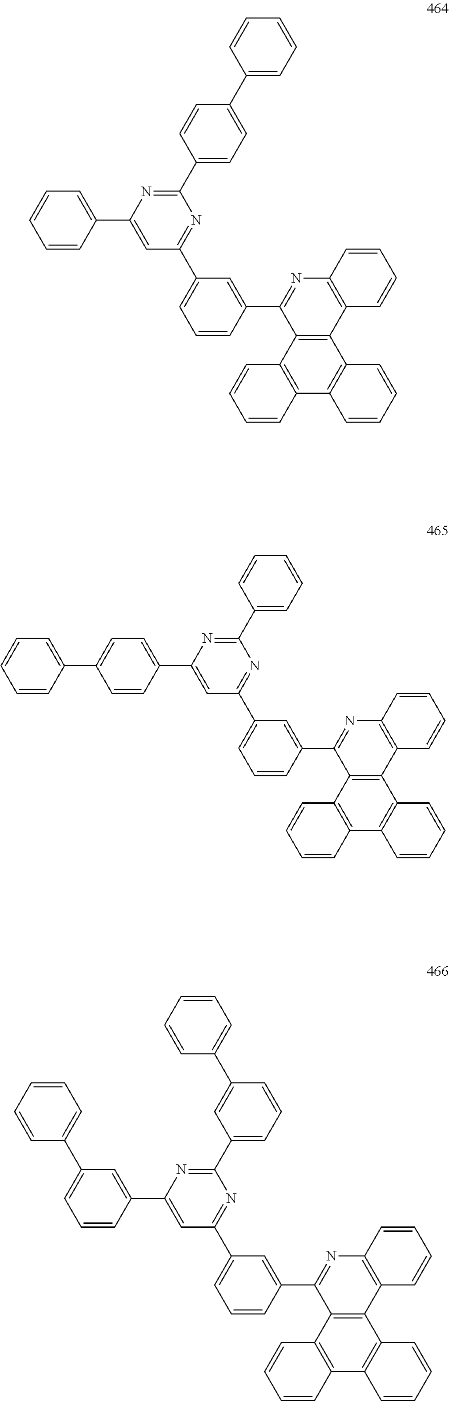 [SMILES: c1ccc(-c2ccc(-c3nc(-c4ccccc4)cc(-c4cccc(-c5nc6ccccc6c6c7ccccc7c7ccccc7c56)c4)n3)cc2)cc1 ; c1ccc(-c2ccc(-c3cc(-c4cccc(-c5nc6ccccc6c6c7ccccc7c7ccccc7c56)c4)nc(-c4ccccc4)n3)cc2)cc1 ; c1ccc(-c2cccc(-c3cc(-c4cccc(-c5nc6ccccc6c6c7ccccc7c7ccccc7c56)c4)nc(-c4cccc(-c5ccccc5)c4)n3)c2)cc1]

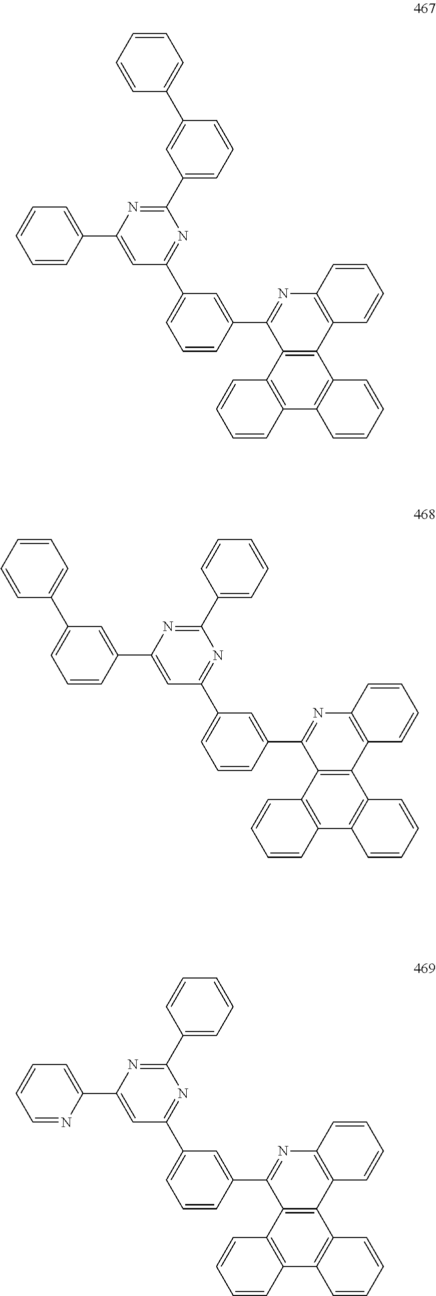 [SMILES: c1ccc(-c2cccc(-c3nc(-c4ccccc4)cc(-c4cccc(-c5nc6ccccc6c6c7ccccc7c7ccccc7c56)c4)n3)c2)cc1 ; c1ccc(-c2cccc(-c3cc(-c4cccc(-c5nc6ccccc6c6c7ccccc7c7ccccc7c56)c4)nc(-c4ccccc4)n3)c2)cc1 ; c1ccc(-c2nc(-c3cccc(-c4nc5ccccc5c5c6ccccc6c6ccccc6c45)c3)cc(-c3ccccn3)n2)cc1]

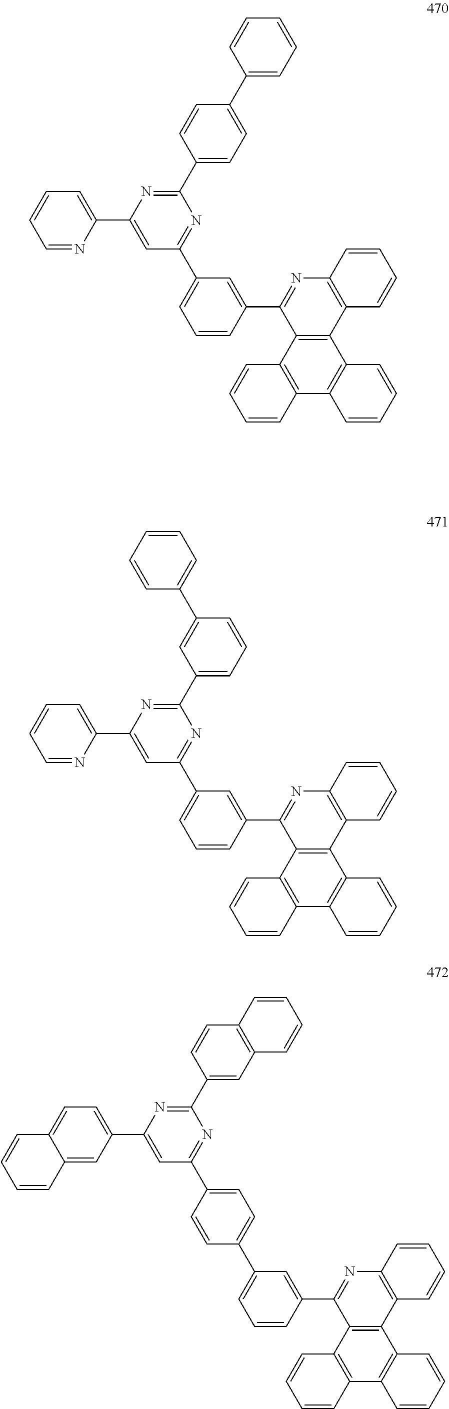 [SMILES: c1ccc(-c2ccc(-c3nc(-c4cccc(-c5nc6ccccc6c6c7ccccc7c7ccccc7c56)c4)cc(-c4ccccn4)n3)cc2)cc1 ; c1ccc(-c2cccc(-c3nc(-c4cccc(-c5nc6ccccc6c6c7ccccc7c7ccccc7c56)c4)cc(-c4ccccn4)n3)c2)cc1 ; c1cc(-c2ccc(-c3cc(-c4ccc5ccccc5c4)nc(-c4ccc5ccccc5c4)n3)cc2)cc(-c2nc3ccccc3c3c4ccccc4c4ccccc4c23)c1]

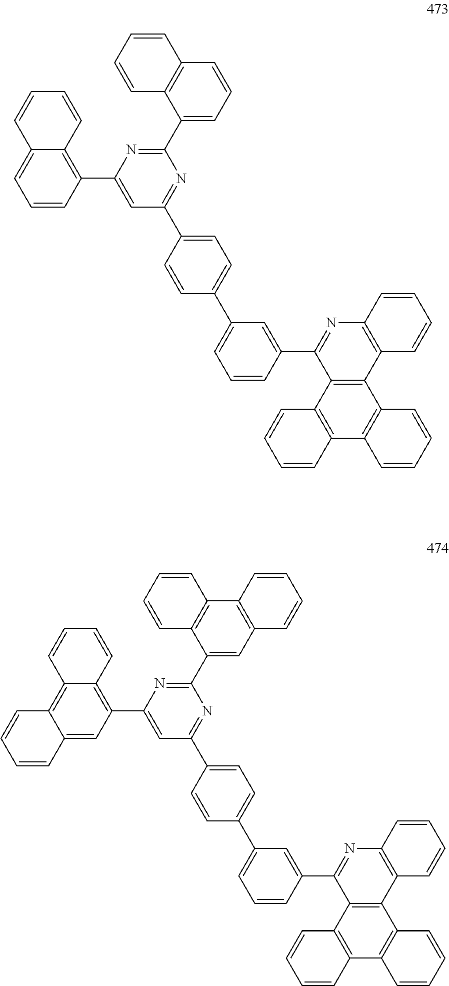 [SMILES: c1cc(-c2ccc(-c3cc(-c4cccc5ccccc45)nc(-c4cccc5ccccc45)n3)cc2)cc(-c2nc3ccccc3c3c4ccccc4c4ccccc4c23)c1 ; c1cc(-c2ccc(-c3cc(-c4cc5ccccc5c5ccccc45)nc(-c4cc5ccccc5c5ccccc45)n3)cc2)cc(-c2nc3ccccc3c3c4ccccc4c4ccccc4c23)c1]

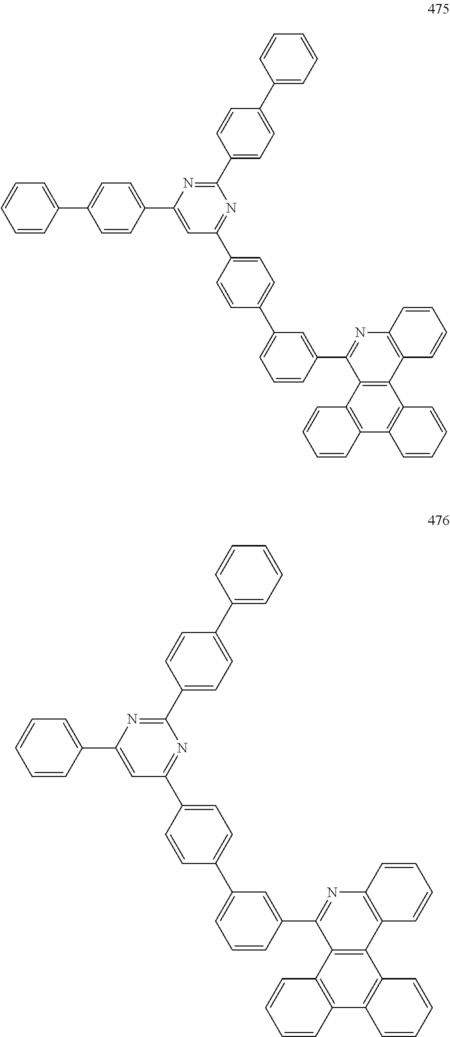 [SMILES: c1ccc(-c2ccc(-c3cc(-c4ccc(-c5cccc(-c6nc7ccccc7c7c8ccccc8c8ccccc8c67)c5)cc4)nc(-c4ccc(-c5ccccc5)cc4)n3)cc2)cc1 ; c1ccc(-c2ccc(-c3nc(-c4ccccc4)cc(-c4ccc(-c5cccc(-c6nc7ccccc7c7c8ccccc8c8ccccc8c67)c5)cc4)n3)cc2)cc1]

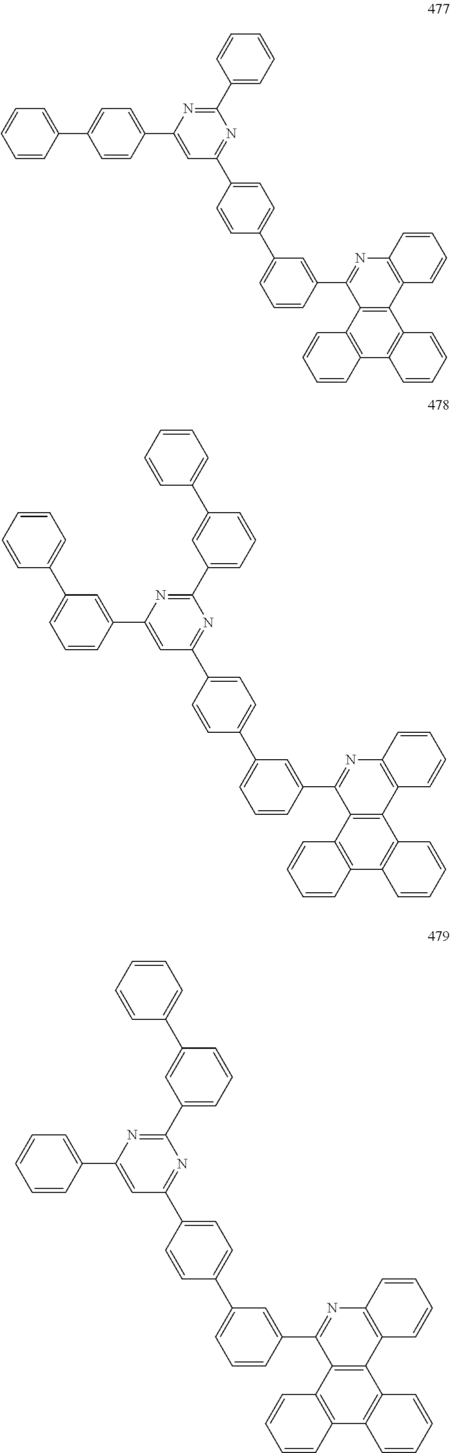 [SMILES: c1ccc(-c2ccc(-c3cc(-c4ccc(-c5cccc(-c6nc7ccccc7c7c8ccccc8c8ccccc8c67)c5)cc4)nc(-c4ccccc4)n3)cc2)cc1 ; c1ccc(-c2cccc(-c3cc(-c4ccc(-c5cccc(-c6nc7ccccc7c7c8ccccc8c8ccccc8c67)c5)cc4)nc(-c4cccc(-c5ccccc5)c4)n3)c2)cc1 ; c1ccc(-c2cccc(-c3nc(-c4ccccc4)cc(-c4ccc(-c5cccc(-c6nc7ccccc7c7c8ccccc8c8ccccc8c67)c5)cc4)n3)c2)cc1]

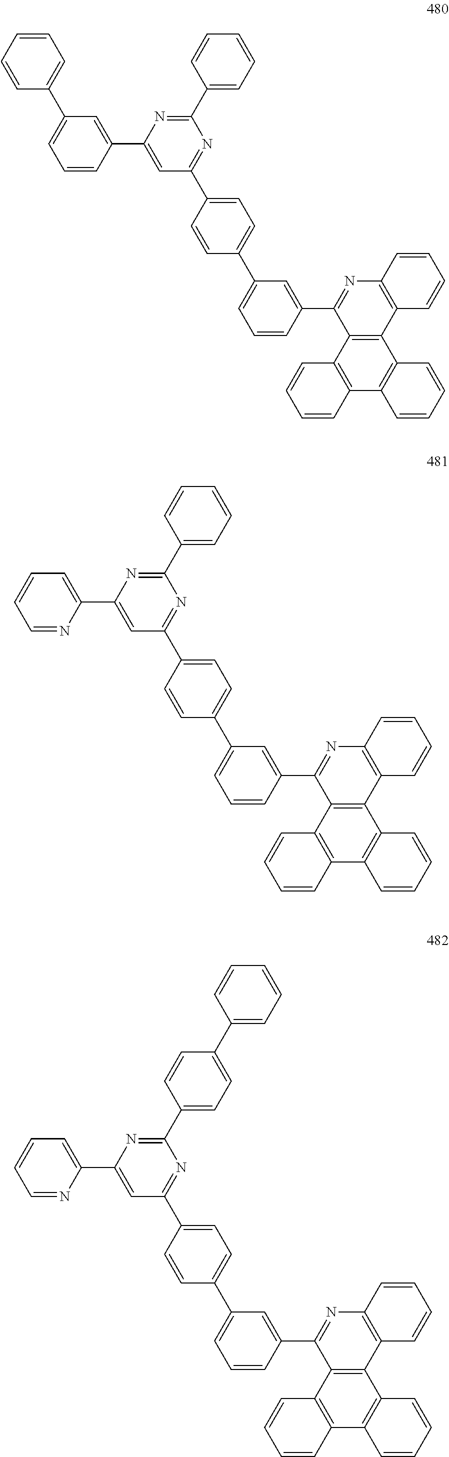 [SMILES: c1ccc(-c2cccc(-c3cc(-c4ccc(-c5cccc(-c6nc7ccccc7c7c8ccccc8c8ccccc8c67)c5)cc4)nc(-c4ccccc4)n3)c2)cc1 ; c1ccc(-c2nc(-c3ccc(-c4cccc(-c5nc6ccccc6c6c7ccccc7c7ccccc7c56)c4)cc3)cc(-c3ccccn3)n2)cc1 ; c1ccc(-c2ccc(-c3nc(-c4ccc(-c5cccc(-c6nc7ccccc7c7c8ccccc8c8ccccc8c67)c5)cc4)cc(-c4ccccn4)n3)cc2)cc1]

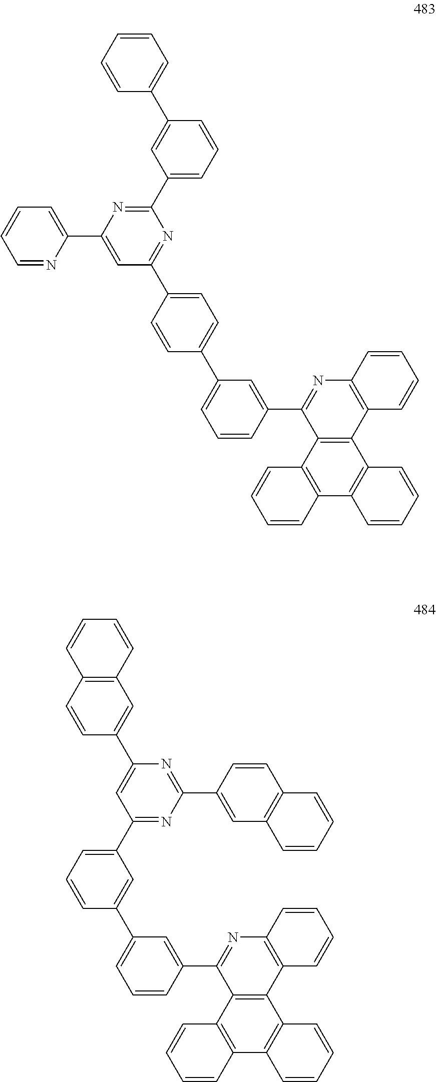 [SMILES: c1ccc(-c2cccc(-c3nc(-c4ccc(-c5cccc(-c6nc7ccccc7c7c8ccccc8c8ccccc8c67)c5)cc4)cc(-c4ccccn4)n3)c2)cc1 ; c1cc(-c2cccc(-c3nc4ccccc4c4c5ccccc5c5ccccc5c34)c2)cc(-c2cc(-c3ccc4ccccc4c3)nc(-c3ccc4ccccc4c3)n2)c1]

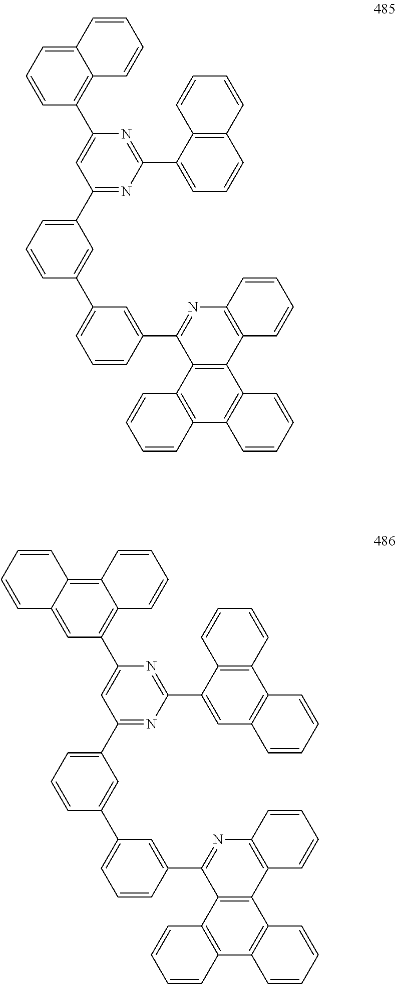 [SMILES: c1cc(-c2cccc(-c3nc4ccccc4c4c5ccccc5c5ccccc5c34)c2)cc(-c2cc(-c3cccc4ccccc34)nc(-c3cccc4ccccc34)n2)c1 ; c1cc(-c2cccc(-c3nc4ccccc4c4c5ccccc5c5ccccc5c34)c2)cc(-c2cc(-c3cc4ccccc4c4ccccc34)nc(-c3cc4ccccc4c4ccccc34)n2)c1]

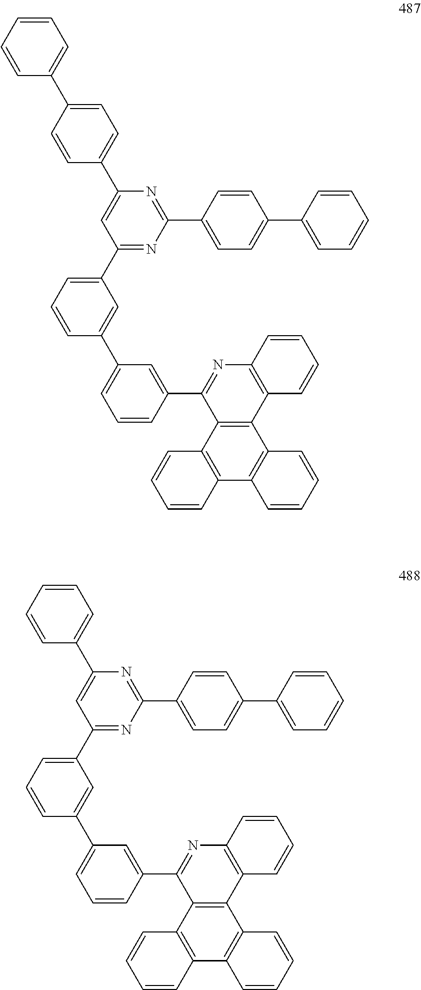 [SMILES: c1ccc(-c2ccc(-c3cc(-c4cccc(-c5cccc(-c6nc7ccccc7c7c8ccccc8c8ccccc8c67)c5)c4)nc(-c4ccc(-c5ccccc5)cc4)n3)cc2)cc1 ; c1ccc(-c2ccc(-c3nc(-c4ccccc4)cc(-c4cccc(-c5cccc(-c6nc7ccccc7c7c8ccccc8c8ccccc8c67)c5)c4)n3)cc2)cc1]

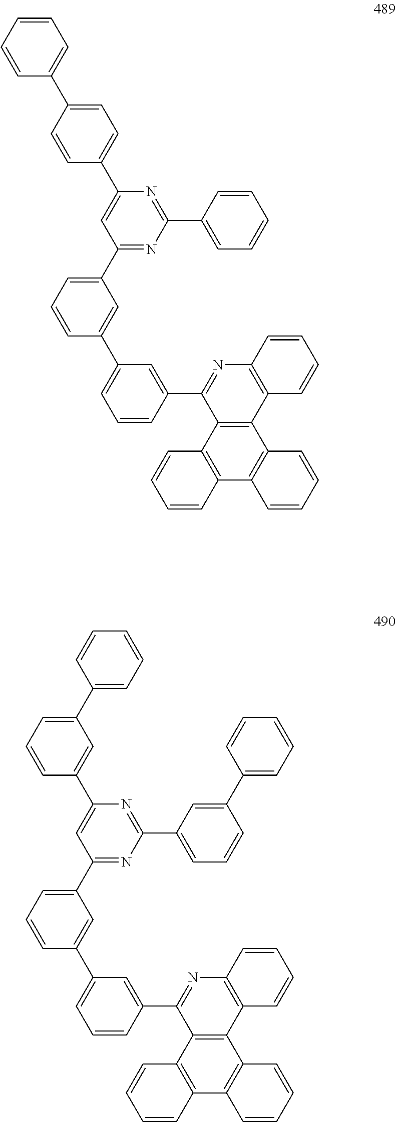 [SMILES: c1ccc(-c2ccc(-c3cc(-c4cccc(-c5cccc(-c6nc7ccccc7c7c8ccccc8c8ccccc8c67)c5)c4)nc(-c4ccccc4)n3)cc2)cc1 ; c1ccc(-c2cccc(-c3cc(-c4cccc(-c5cccc(-c6nc7ccccc7c7c8ccccc8c8ccccc8c67)c5)c4)nc(-c4cccc(-c5ccccc5)c4)n3)c2)cc1]

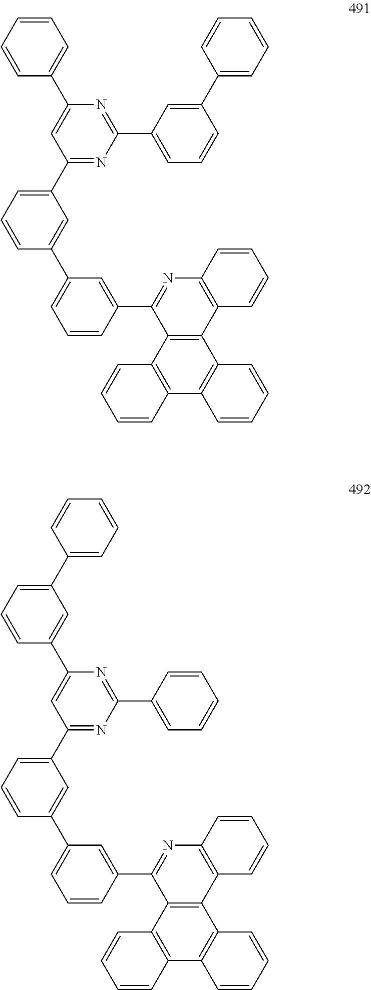 [SMILES: c1ccc(-c2cccc(-c3nc(-c4ccccc4)cc(-c4cccc(-c5cccc(-c6nc7ccccc7c7c8ccccc8c8ccccc8c67)c5)c4)n3)c2)cc1 ; c1ccc(-c2cccc(-c3cc(-c4cccc(-c5cccc(-c6nc7ccccc7c7c8ccccc8c8ccccc8c67)c5)c4)nc(-c4ccccc4)n3)c2)cc1]

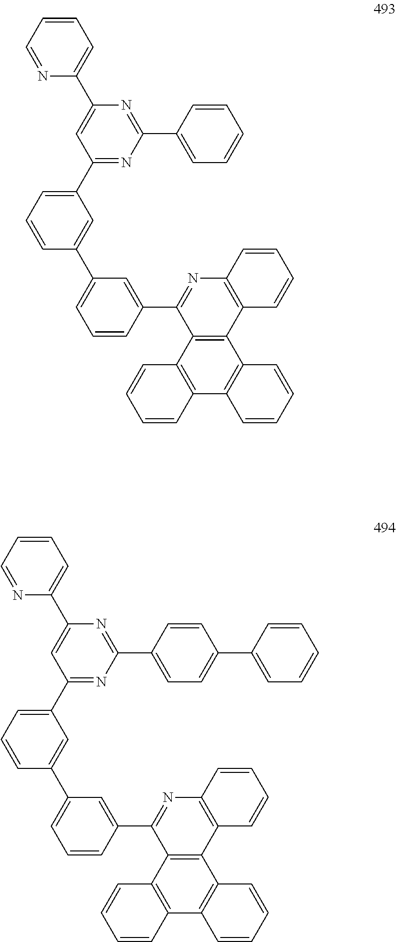 [SMILES: c1ccc(-c2nc(-c3cccc(-c4cccc(-c5nc6ccccc6c6c7ccccc7c7ccccc7c56)c4)c3)cc(-c3ccccn3)n2)cc1 ; c1ccc(-c2ccc(-c3nc(-c4cccc(-c5cccc(-c6nc7ccccc7c7c8ccccc8c8ccccc8c67)c5)c4)cc(-c4ccccn4)n3)cc2)cc1]

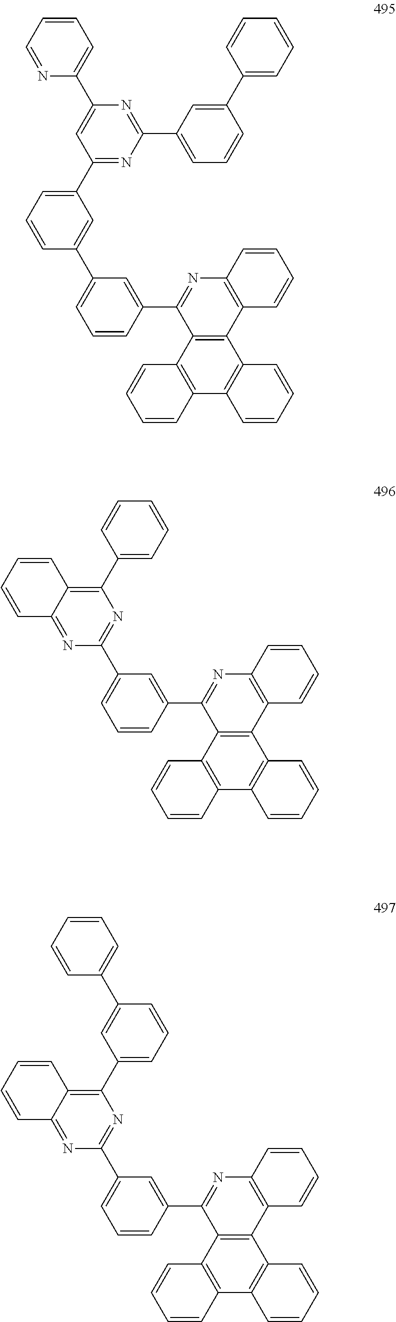 [SMILES: c1ccc(-c2cccc(-c3nc(-c4cccc(-c5cccc(-c6nc7ccccc7c7c8ccccc8c8ccccc8c67)c5)c4)cc(-c4ccccn4)n3)c2)cc1 ; c1ccc(-c2nc(-c3cccc(-c4nc5ccccc5c5c6ccccc6c6ccccc6c45)c3)nc3ccccc23)cc1 ; c1ccc(-c2cccc(-c3nc(-c4cccc(-c5nc6ccccc6c6c7ccccc7c7ccccc7c56)c4)nc4ccccc34)c2)cc1]

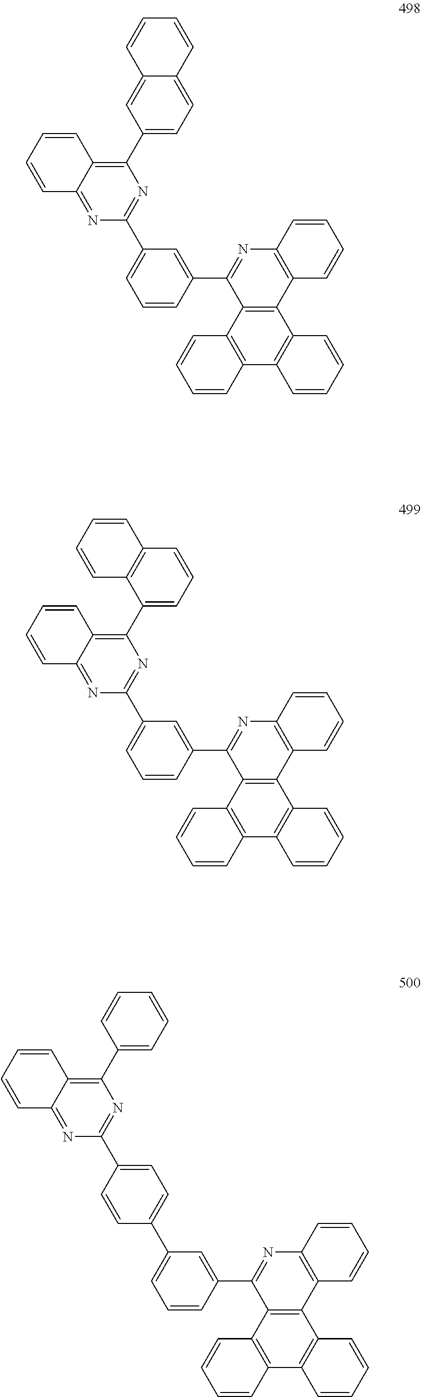 [SMILES: c1cc(-c2nc(-c3ccc4ccccc4c3)c3ccccc3n2)cc(-c2nc3ccccc3c3c4ccccc4c4ccccc4c23)c1 ; c1cc(-c2nc(-c3cccc4ccccc34)c3ccccc3n2)cc(-c2nc3ccccc3c3c4ccccc4c4ccccc4c23)c1 ; c1ccc(-c2nc(-c3ccc(-c4cccc(-c5nc6ccccc6c6c7ccccc7c7ccccc7c56)c4)cc3)nc3ccccc23)cc1]

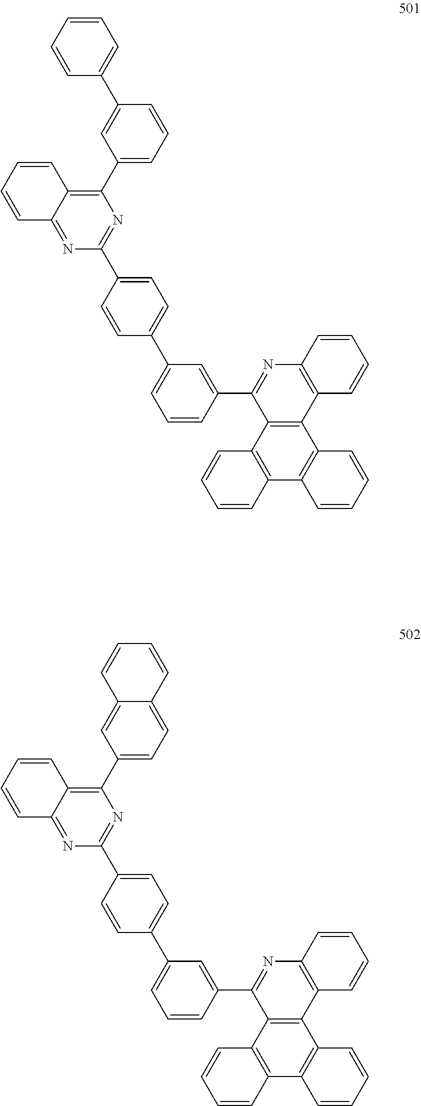 [SMILES: c1ccc(-c2cccc(-c3nc(-c4ccc(-c5cccc(-c6nc7ccccc7c7c8ccccc8c8ccccc8c67)c5)cc4)nc4ccccc34)c2)cc1 ; c1cc(-c2ccc(-c3nc(-c4ccc5ccccc5c4)c4ccccc4n3)cc2)cc(-c2nc3ccccc3c3c4ccccc4c4ccccc4c23)c1]

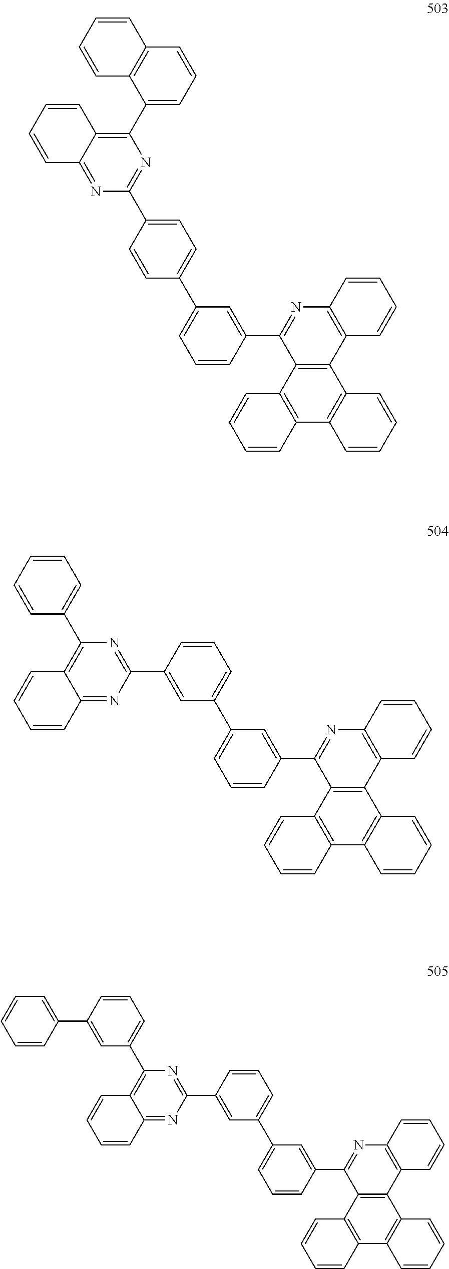 [SMILES: c1cc(-c2ccc(-c3nc(-c4cccc5ccccc45)c4ccccc4n3)cc2)cc(-c2nc3ccccc3c3c4ccccc4c4ccccc4c23)c1 ; c1ccc(-c2nc(-c3cccc(-c4cccc(-c5nc6ccccc6c6c7ccccc7c7ccccc7c56)c4)c3)nc3ccccc23)cc1 ; c1ccc(-c2cccc(-c3nc(-c4cccc(-c5cccc(-c6nc7ccccc7c7c8ccccc8c8ccccc8c67)c5)c4)nc4ccccc34)c2)cc1]

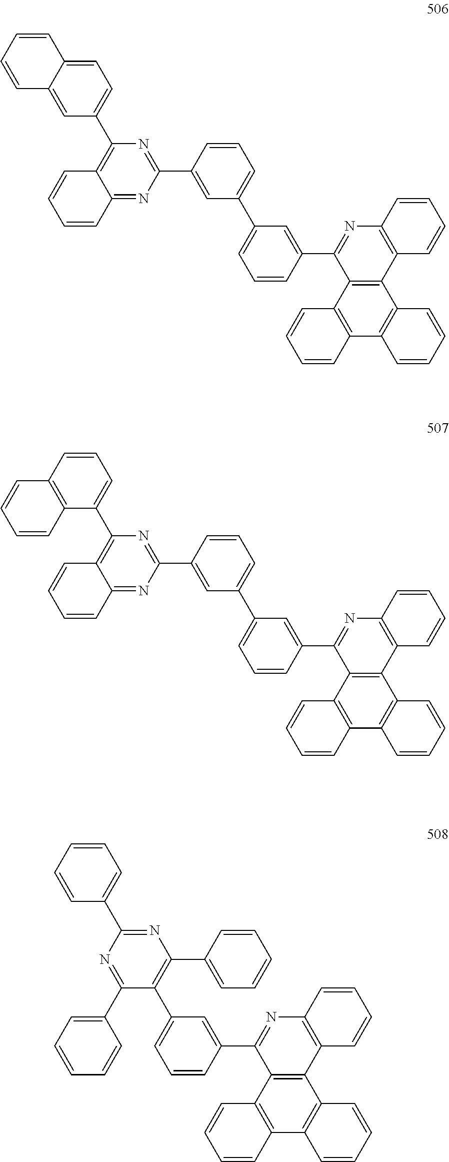 [SMILES: c1cc(-c2cccc(-c3nc4ccccc4c4c5ccccc5c5ccccc5c34)c2)cc(-c2nc(-c3ccc4ccccc4c3)c3ccccc3n2)c1 ; c1cc(-c2cccc(-c3nc4ccccc4c4c5ccccc5c5ccccc5c34)c2)cc(-c2nc(-c3cccc4ccccc34)c3ccccc3n2)c1 ; c1ccc(-c2nc(-c3ccccc3)c(-c3cccc(-c4nc5ccccc5c5c6ccccc6c6ccccc6c45)c3)c(-c3ccccc3)n2)cc1]

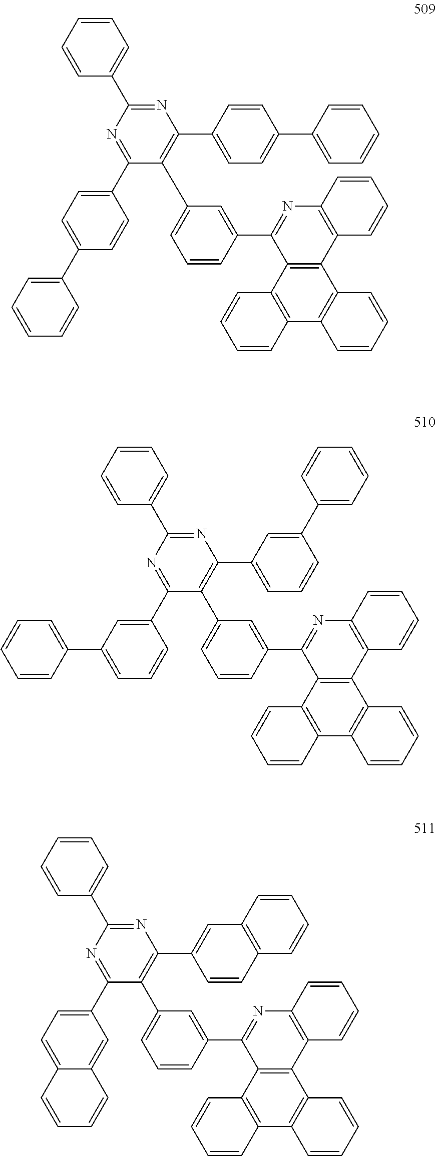 [SMILES: c1ccc(-c2ccc(-c3nc(-c4ccccc4)nc(-c4ccc(-c5ccccc5)cc4)c3-c3cccc(-c4nc5ccccc5c5c6ccccc6c6ccccc6c45)c3)cc2)cc1 ; c1ccc(-c2cccc(-c3nc(-c4ccccc4)nc(-c4cccc(-c5ccccc5)c4)c3-c3cccc(-c4nc5ccccc5c5c6ccccc6c6ccccc6c45)c3)c2)cc1 ; c1ccc(-c2nc(-c3ccc4ccccc4c3)c(-c3cccc(-c4nc5ccccc5c5c6ccccc6c6ccccc6c45)c3)c(-c3ccc4ccccc4c3)n2)cc1]

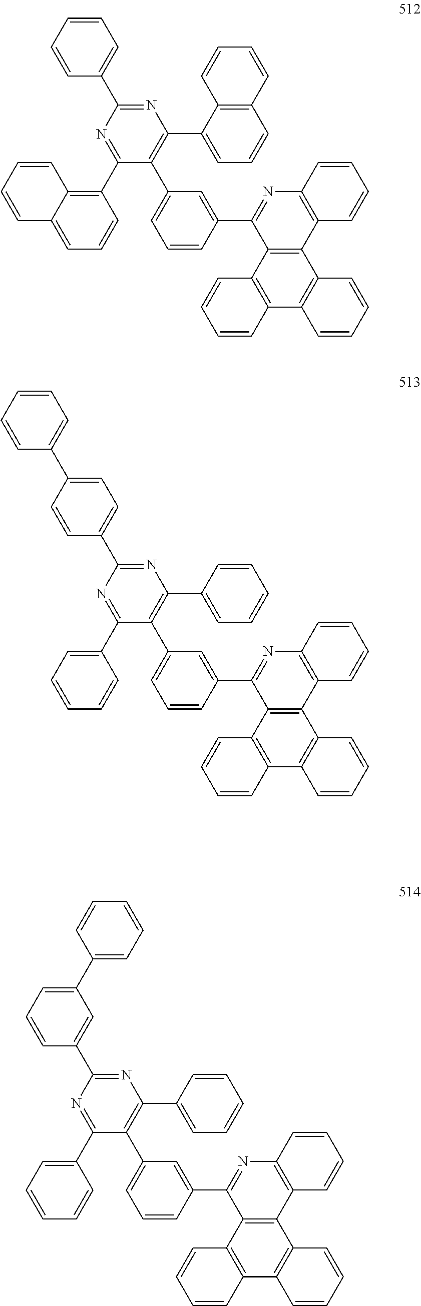 [SMILES: c1ccc(-c2nc(-c3cccc4ccccc34)c(-c3cccc(-c4nc5ccccc5c5c6ccccc6c6ccccc6c45)c3)c(-c3cccc4ccccc34)n2)cc1 ; c1ccc(-c2ccc(-c3nc(-c4ccccc4)c(-c4cccc(-c5nc6ccccc6c6c7ccccc7c7ccccc7c56)c4)c(-c4ccccc4)n3)cc2)cc1 ; c1ccc(-c2cccc(-c3nc(-c4ccccc4)c(-c4cccc(-c5nc6ccccc6c6c7ccccc7c7ccccc7c56)c4)c(-c4ccccc4)n3)c2)cc1]

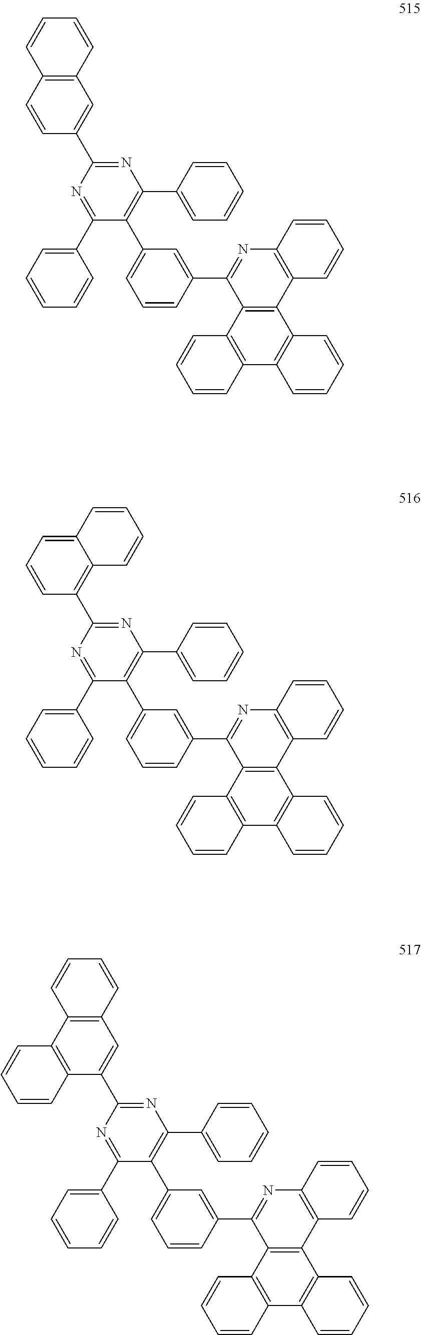 [SMILES: c1ccc(-c2nc(-c3ccc4ccccc4c3)nc(-c3ccccc3)c2-c2cccc(-c3nc4ccccc4c4c5ccccc5c5ccccc5c34)c2)cc1 ; c1ccc(-c2nc(-c3cccc4ccccc34)nc(-c3ccccc3)c2-c2cccc(-c3nc4ccccc4c4c5ccccc5c5ccccc5c34)c2)cc1 ; c1ccc(-c2nc(-c3cc4ccccc4c4ccccc34)nc(-c3ccccc3)c2-c2cccc(-c3nc4ccccc4c4c5ccccc5c5ccccc5c34)c2)cc1]

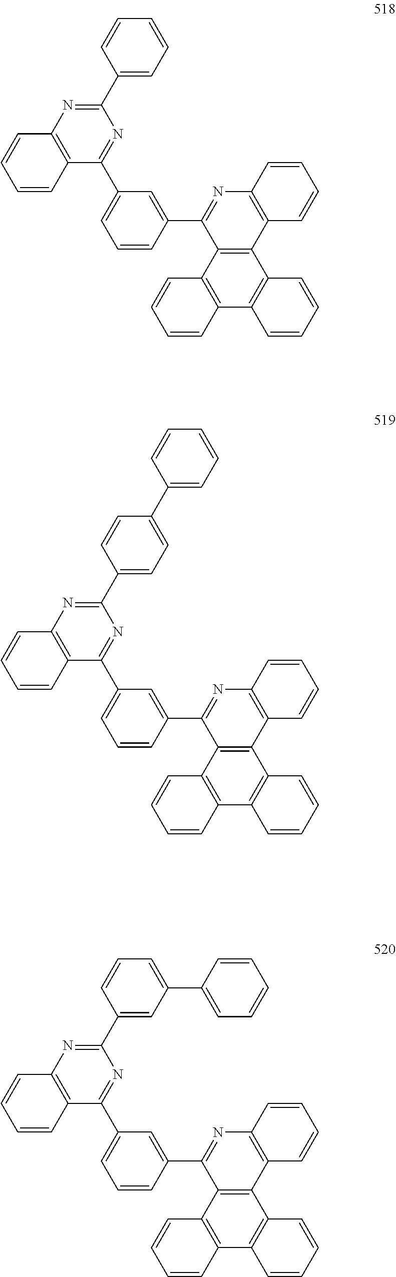 [SMILES: c1ccc(-c2nc(-c3cccc(-c4nc5ccccc5c5c6ccccc6c6ccccc6c45)c3)c3ccccc3n2)cc1 ; c1ccc(-c2ccc(-c3nc(-c4cccc(-c5nc6ccccc6c6c7ccccc7c7ccccc7c56)c4)c4ccccc4n3)cc2)cc1 ; c1ccc(-c2cccc(-c3nc(-c4cccc(-c5nc6ccccc6c6c7ccccc7c7ccccc7c56)c4)c4ccccc4n3)c2)cc1]

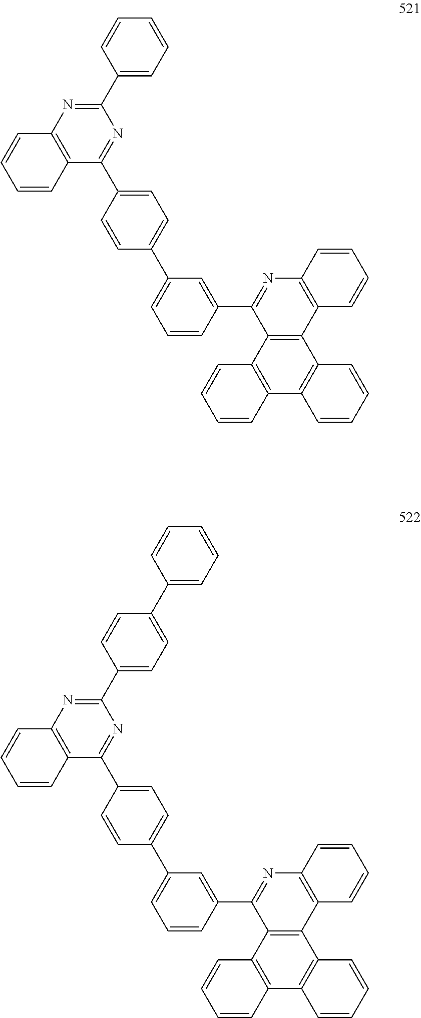 [SMILES: c1ccc(-c2nc(-c3ccc(-c4cccc(-c5nc6ccccc6c6c7ccccc7c7ccccc7c56)c4)cc3)c3ccccc3n2)cc1 ; c1ccc(-c2ccc(-c3nc(-c4ccc(-c5cccc(-c6nc7ccccc7c7c8ccccc8c8ccccc8c67)c5)cc4)c4ccccc4n3)cc2)cc1]

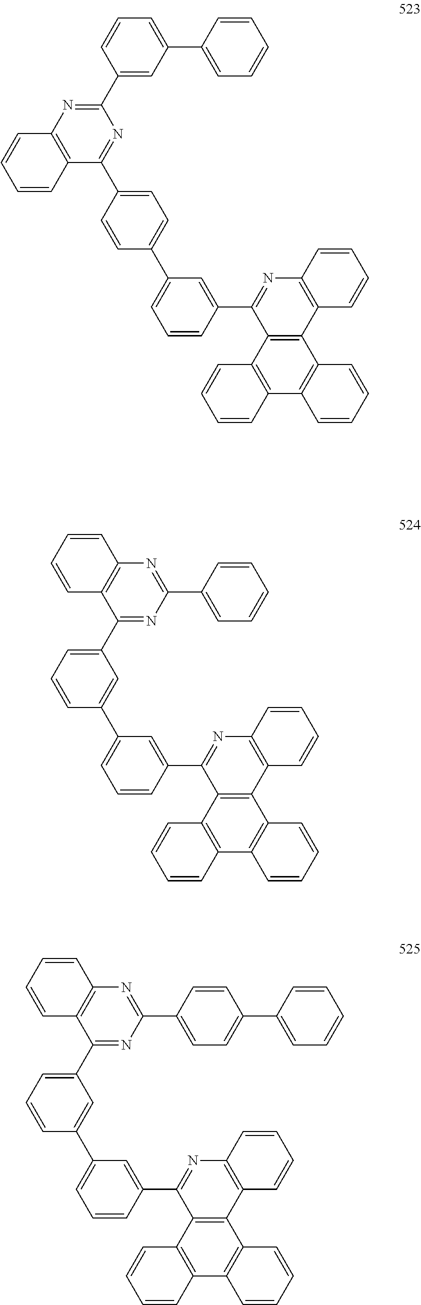 [SMILES: c1ccc(-c2cccc(-c3nc(-c4ccc(-c5cccc(-c6nc7ccccc7c7c8ccccc8c8ccccc8c67)c5)cc4)c4ccccc4n3)c2)cc1 ; c1ccc(-c2nc(-c3cccc(-c4cccc(-c5nc6ccccc6c6c7ccccc7c7ccccc7c56)c4)c3)c3ccccc3n2)cc1 ; c1ccc(-c2ccc(-c3nc(-c4cccc(-c5cccc(-c6nc7ccccc7c7c8ccccc8c8ccccc8c67)c5)c4)c4ccccc4n3)cc2)cc1]

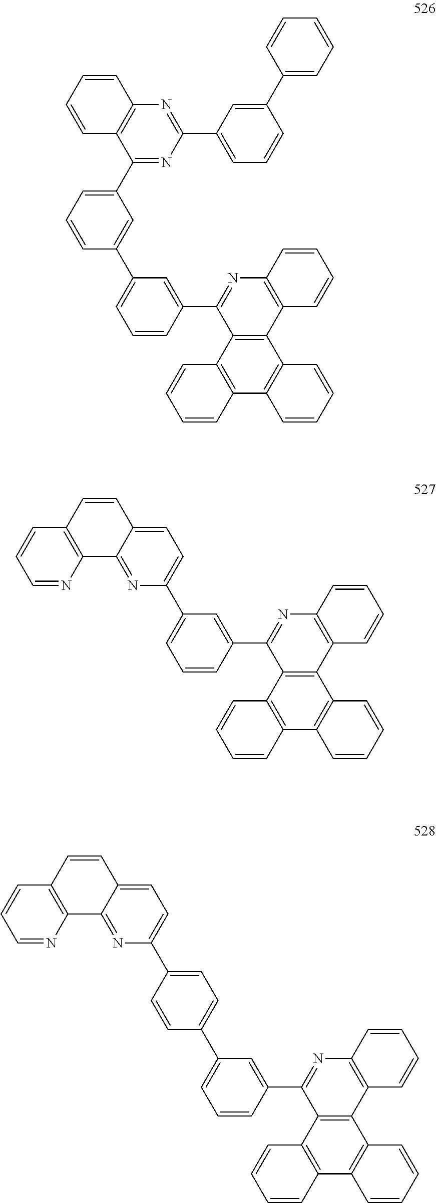 [SMILES: c1ccc(-c2cccc(-c3nc(-c4cccc(-c5cccc(-c6nc7ccccc7c7c8ccccc8c8ccccc8c67)c5)c4)c4ccccc4n3)c2)cc1 ; c1cc(-c2ccc3ccc4cccnc4c3n2)cc(-c2nc3ccccc3c3c4ccccc4c4ccccc4c23)c1 ; c1cc(-c2ccc(-c3ccc4ccc5cccnc5c4n3)cc2)cc(-c2nc3ccccc3c3c4ccccc4c4ccccc4c23)c1]

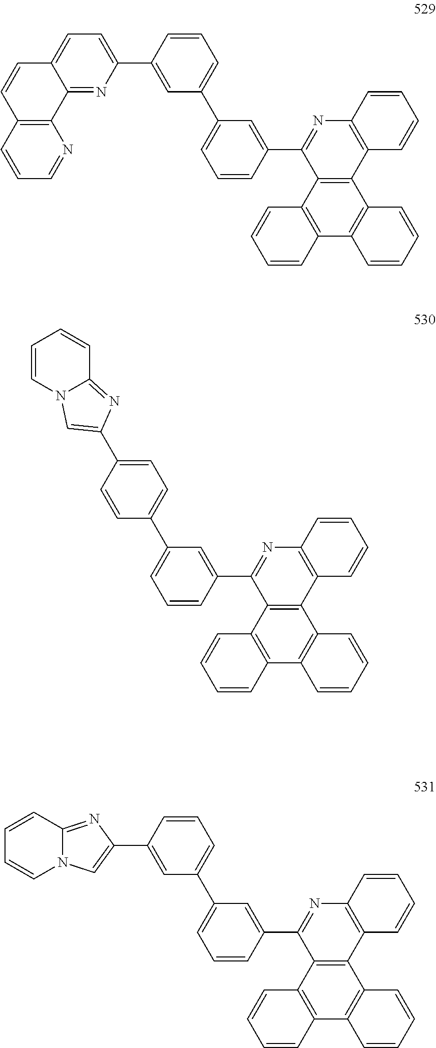 [SMILES: c1cc(-c2cccc(-c3nc4ccccc4c4c5ccccc5c5ccccc5c34)c2)cc(-c2ccc3ccc4cccnc4c3n2)c1 ; c1cc(-c2ccc(-c3cn4ccccc4n3)cc2)cc(-c2nc3ccccc3c3c4ccccc4c4ccccc4c23)c1 ; c1cc(-c2cccc(-c3nc4ccccc4c4c5ccccc5c5ccccc5c34)c2)cc(-c2cn3ccccc3n2)c1]

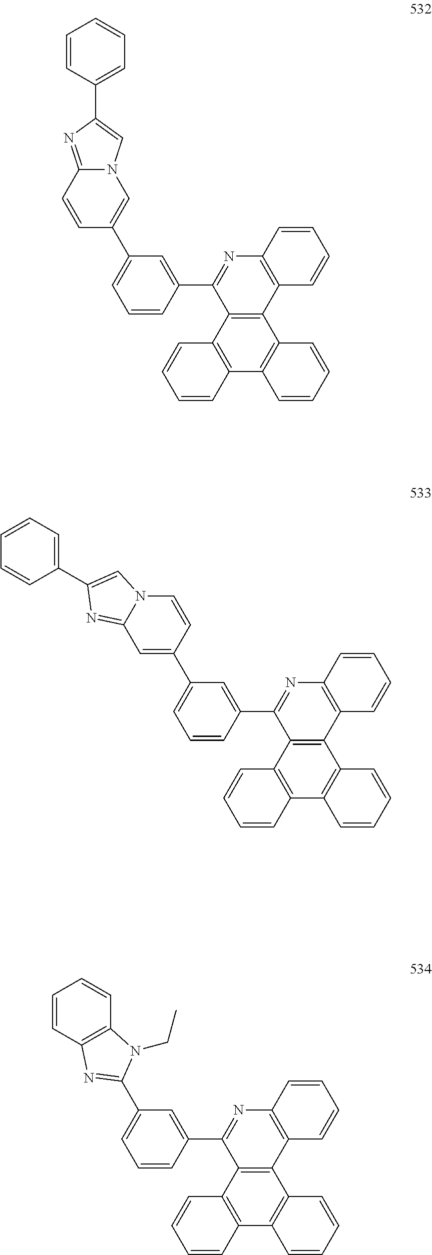 [SMILES: c1ccc(-c2cn3cc(-c4cccc(-c5nc6ccccc6c6c7ccccc7c7ccccc7c56)c4)ccc3n2)cc1 ; c1ccc(-c2cn3ccc(-c4cccc(-c5nc6ccccc6c6c7ccccc7c7ccccc7c56)c4)cc3n2)cc1 ; CCn1c(-c2cccc(-c3nc4ccccc4c4c5ccccc5c5ccccc5c34)c2)nc2ccccc21]

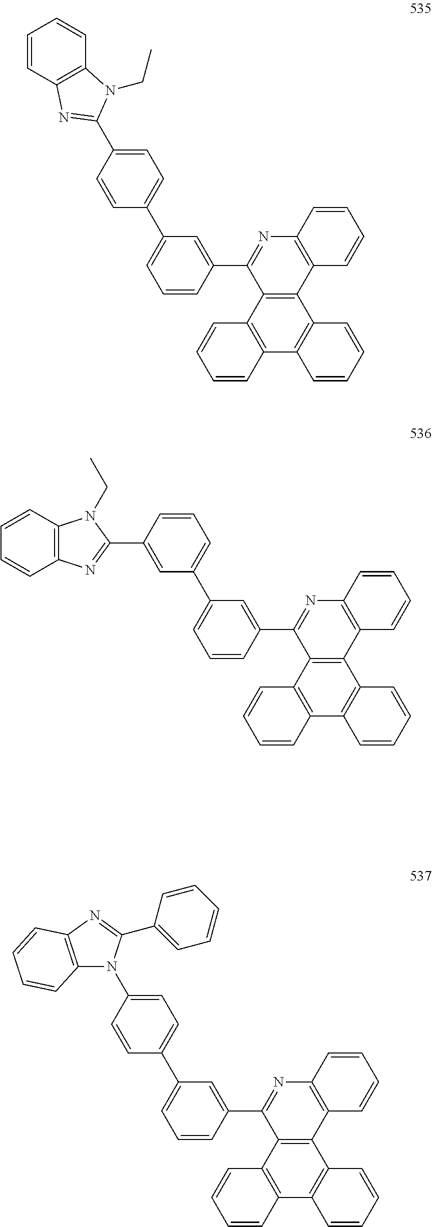 [SMILES: CCn1c(-c2ccc(-c3cccc(-c4nc5ccccc5c5c6ccccc6c6ccccc6c45)c3)cc2)nc2ccccc21 ; CCn1c(-c2cccc(-c3cccc(-c4nc5ccccc5c5c6ccccc6c6ccccc6c45)c3)c2)nc2ccccc21 ; c1ccc(-c2nc3ccccc3n2-c2ccc(-c3cccc(-c4nc5ccccc5c5c6ccccc6c6ccccc6c45)c3)cc2)cc1]

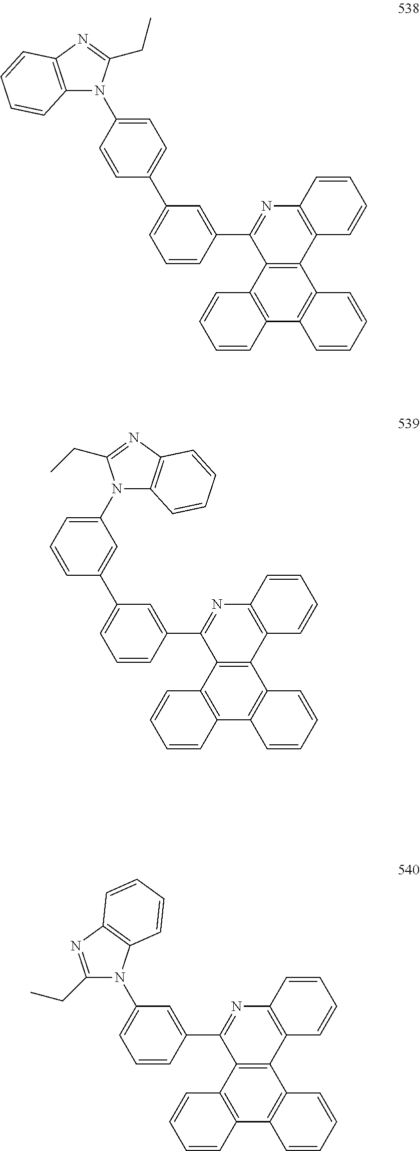 [SMILES: CCc1nc2ccccc2n1-c1ccc(-c2cccc(-c3nc4ccccc4c4c5ccccc5c5ccccc5c34)c2)cc1 ; CCc1nc2ccccc2n1-c1cccc(-c2cccc(-c3nc4ccccc4c4c5ccccc5c5ccccc5c34)c2)c1 ; CCc1nc2ccccc2n1-c1cccc(-c2nc3ccccc3c3c4ccccc4c4ccccc4c23)c1]

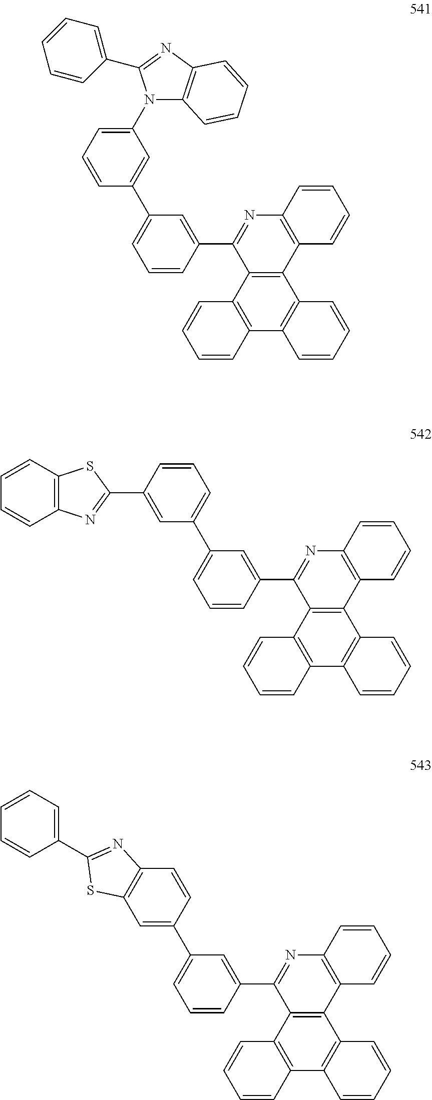 [SMILES: c1ccc(-c2nc3ccccc3n2-c2cccc(-c3cccc(-c4nc5ccccc5c5c6ccccc6c6ccccc6c45)c3)c2)cc1 ; c1cc(-c2cccc(-c3nc4ccccc4c4c5ccccc5c5ccccc5c34)c2)cc(-c2nc3ccccc3s2)c1 ; c1ccc(-c2nc3ccc(-c4cccc(-c5nc6ccccc6c6c7ccccc7c7ccccc7c56)c4)cc3s2)cc1]

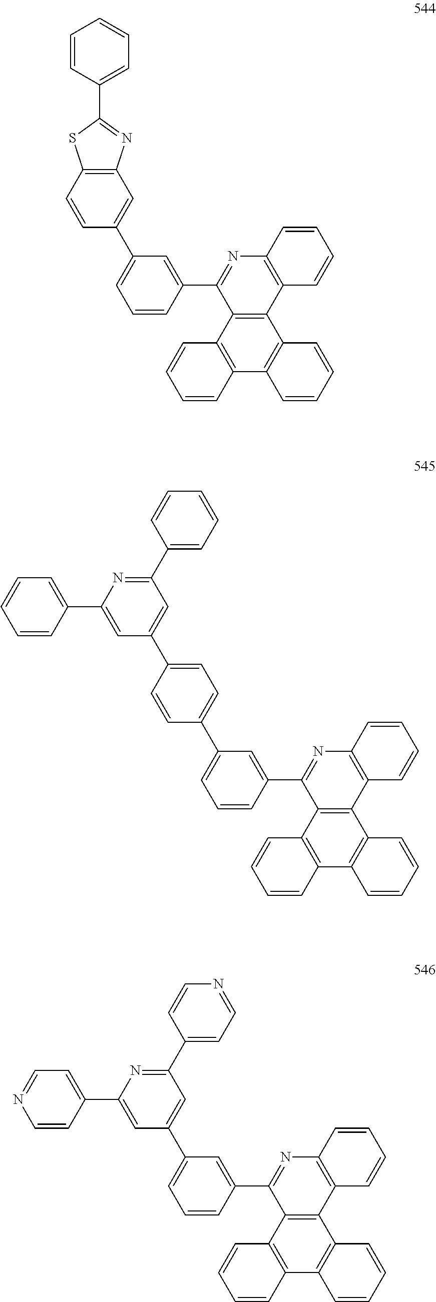 [SMILES: c1ccc(-c2nc3cc(-c4cccc(-c5nc6ccccc6c6c7ccccc7c7ccccc7c56)c4)ccc3s2)cc1 ; c1ccc(-c2cc(-c3ccc(-c4cccc(-c5nc6ccccc6c6c7ccccc7c7ccccc7c56)c4)cc3)cc(-c3ccccc3)n2)cc1 ; c1cc(-c2cc(-c3ccncc3)nc(-c3ccncc3)c2)cc(-c2nc3ccccc3c3c4ccccc4c4ccccc4c23)c1]

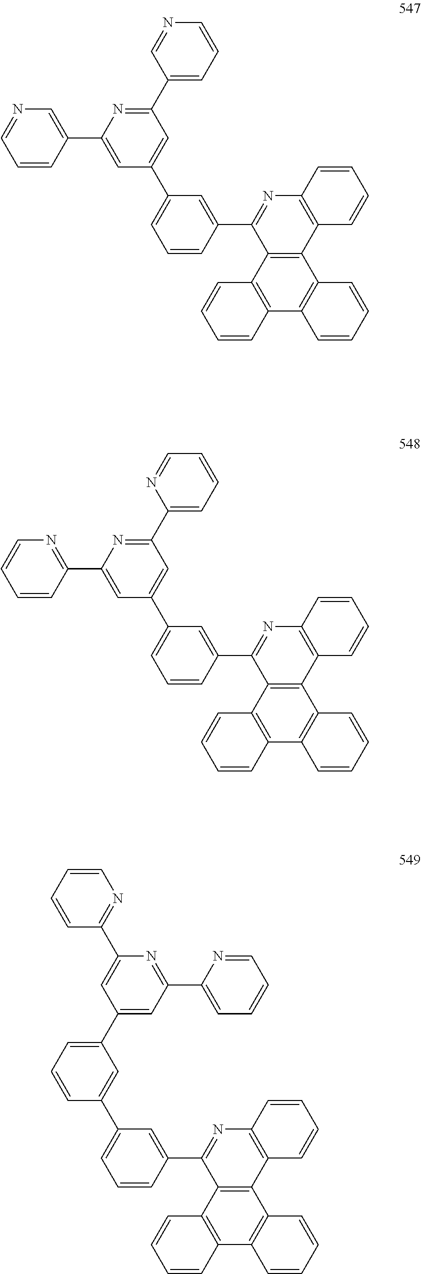 [SMILES: c1cncc(-c2cc(-c3cccc(-c4nc5ccccc5c5c6ccccc6c6ccccc6c45)c3)cc(-c3cccnc3)n2)c1 ; c1ccc(-c2cc(-c3cccc(-c4nc5ccccc5c5c6ccccc6c6ccccc6c45)c3)cc(-c3ccccn3)n2)nc1 ; c1ccc(-c2cc(-c3cccc(-c4cccc(-c5nc6ccccc6c6c7ccccc7c7ccccc7c56)c4)c3)cc(-c3ccccn3)n2)nc1]

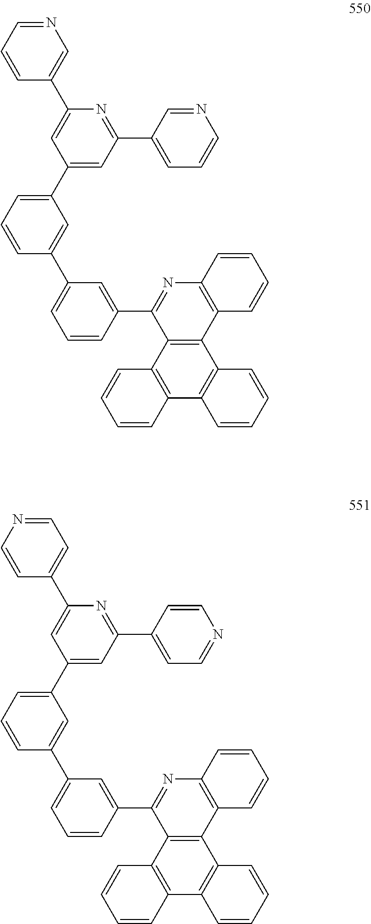 [SMILES: c1cc(-c2cccc(-c3nc4ccccc4c4c5ccccc5c5ccccc5c34)c2)cc(-c2cc(-c3cccnc3)nc(-c3cccnc3)c2)c1 ; c1cc(-c2cccc(-c3nc4ccccc4c4c5ccccc5c5ccccc5c34)c2)cc(-c2cc(-c3ccncc3)nc(-c3ccncc3)c2)c1]

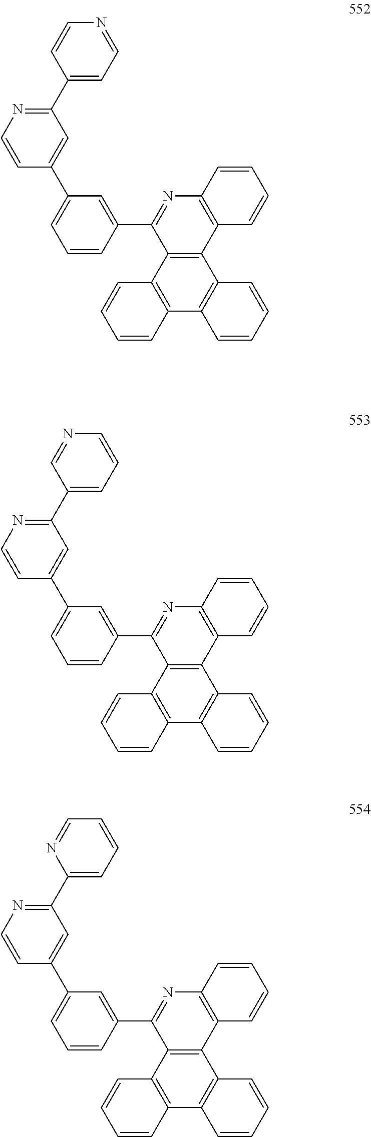 [SMILES: c1cc(-c2ccnc(-c3ccncc3)c2)cc(-c2nc3ccccc3c3c4ccccc4c4ccccc4c23)c1 ; c1cncc(-c2cc(-c3cccc(-c4nc5ccccc5c5c6ccccc6c6ccccc6c45)c3)ccn2)c1 ; c1ccc(-c2cc(-c3cccc(-c4nc5ccccc5c5c6ccccc6c6ccccc6c45)c3)ccn2)nc1]

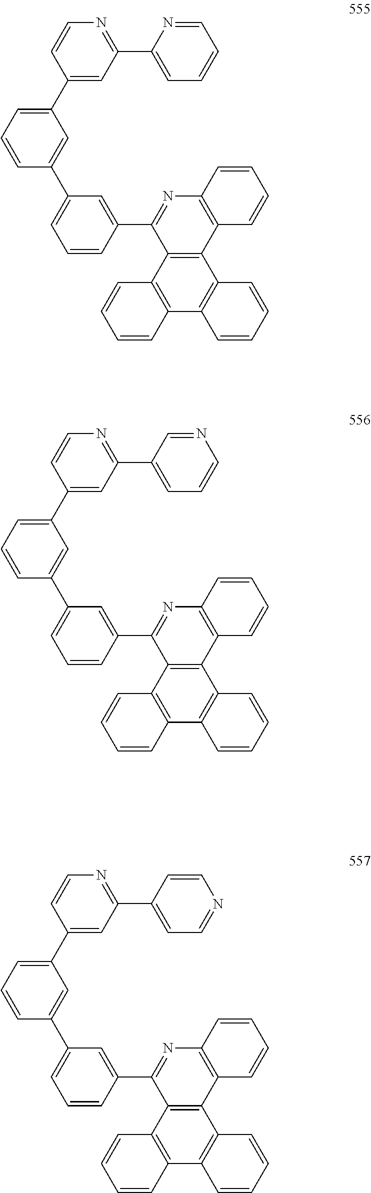 [SMILES: c1ccc(-c2cc(-c3cccc(-c4cccc(-c5nc6ccccc6c6c7ccccc7c7ccccc7c56)c4)c3)ccn2)nc1 ; c1cc(-c2cccc(-c3nc4ccccc4c4c5ccccc5c5ccccc5c34)c2)cc(-c2ccnc(-c3cccnc3)c2)c1 ; c1cc(-c2cccc(-c3nc4ccccc4c4c5ccccc5c5ccccc5c34)c2)cc(-c2ccnc(-c3ccncc3)c2)c1]

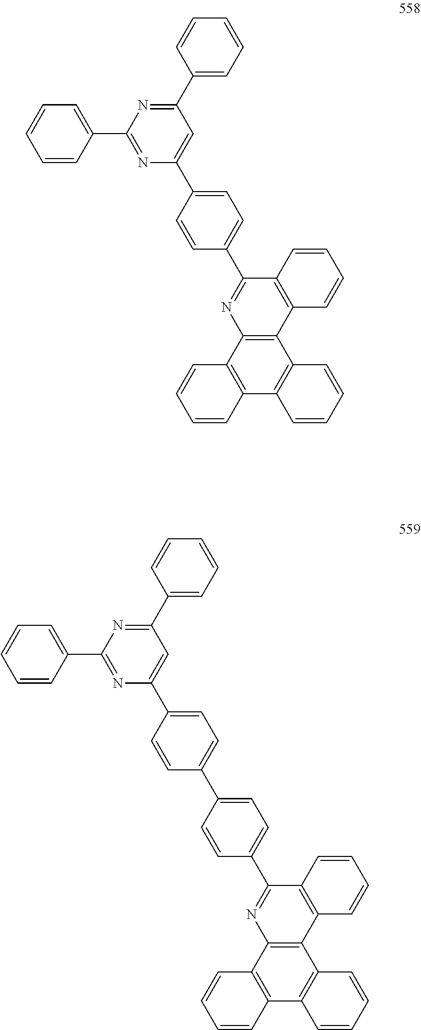 [SMILES: c1ccc(-c2cc(-c3ccc(-c4nc5c6ccccc6c6ccccc6c5c5ccccc45)cc3)nc(-c3ccccc3)n2)cc1 ; c1ccc(-c2cc(-c3ccc(-c4ccc(-c5nc6c7ccccc7c7ccccc7c6c6ccccc56)cc4)cc3)nc(-c3ccccc3)n2)cc1]

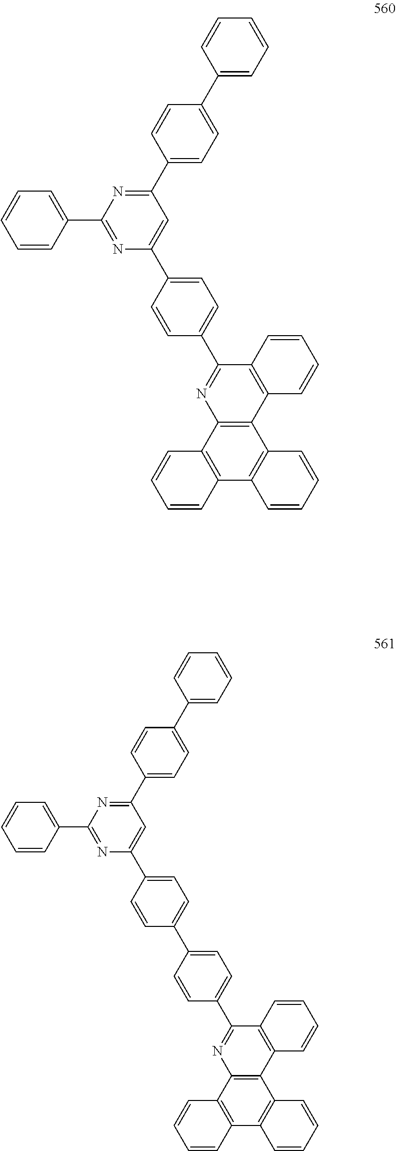 [SMILES: c1ccc(-c2ccc(-c3cc(-c4ccc(-c5nc6c7ccccc7c7ccccc7c6c6ccccc56)cc4)nc(-c4ccccc4)n3)cc2)cc1 ; c1ccc(-c2ccc(-c3cc(-c4ccc(-c5ccc(-c6nc7c8ccccc8c8ccccc8c7c7ccccc67)cc5)cc4)nc(-c4ccccc4)n3)cc2)cc1]

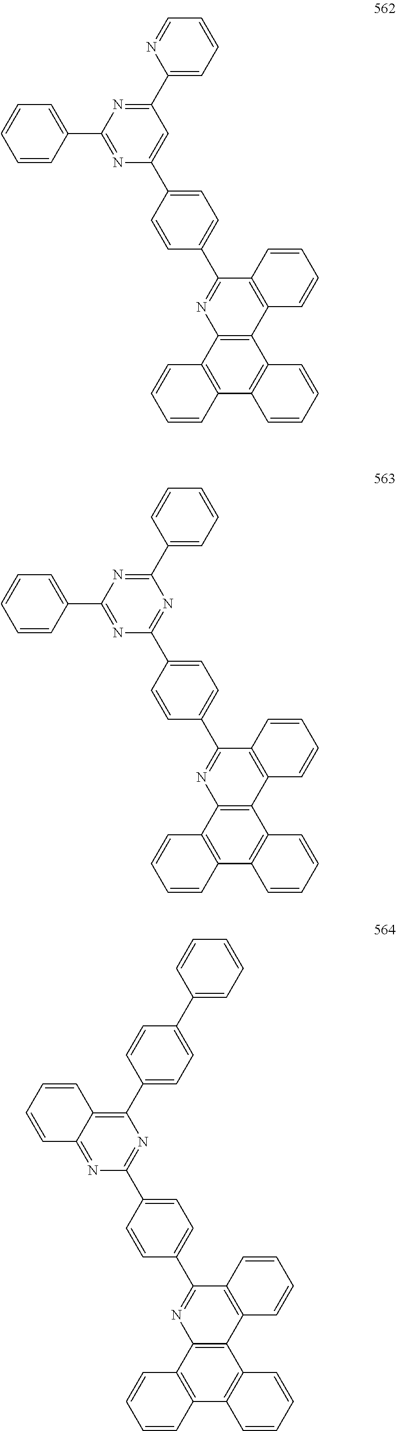 [SMILES: c1ccc(-c2nc(-c3ccc(-c4nc5c6ccccc6c6ccccc6c5c5ccccc45)cc3)cc(-c3ccccn3)n2)cc1 ; c1ccc(-c2nc(-c3ccccc3)nc(-c3ccc(-c4nc5c6ccccc6c6ccccc6c5c5ccccc45)cc3)n2)cc1 ; c1ccc(-c2ccc(-c3nc(-c4ccc(-c5nc6c7ccccc7c7ccccc7c6c6ccccc56)cc4)nc4ccccc34)cc2)cc1]

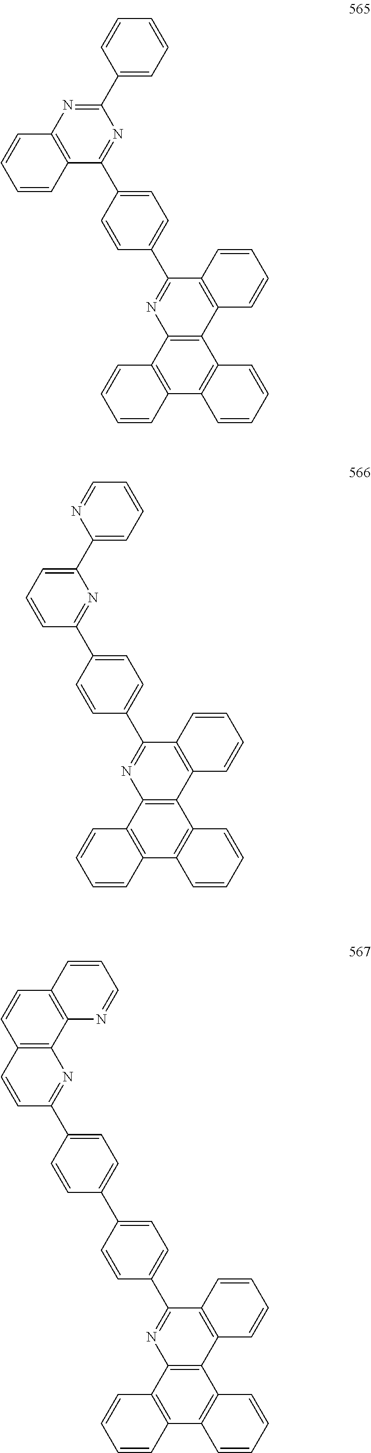 [SMILES: c1ccc(-c2nc(-c3ccc(-c4nc5c6ccccc6c6ccccc6c5c5ccccc45)cc3)c3ccccc3n2)cc1 ; c1ccc(-c2cccc(-c3ccc(-c4nc5c6ccccc6c6ccccc6c5c5ccccc45)cc3)n2)nc1 ; c1cnc2c(c1)ccc1ccc(-c3ccc(-c4ccc(-c5nc6c7ccccc7c7ccccc7c6c6ccccc56)cc4)cc3)nc12]

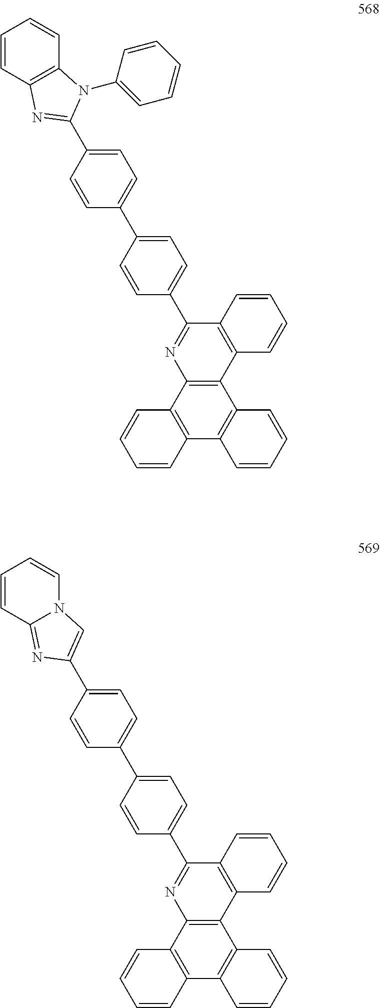 [SMILES: c1ccc(-n2c(-c3ccc(-c4ccc(-c5nc6c7ccccc7c7ccccc7c6c6ccccc56)cc4)cc3)nc3ccccc32)cc1 ; c1ccc2c(c1)c(-c1ccc(-c3ccc(-c4cn5ccccc5n4)cc3)cc1)nc1c3ccccc3c3ccccc3c21]

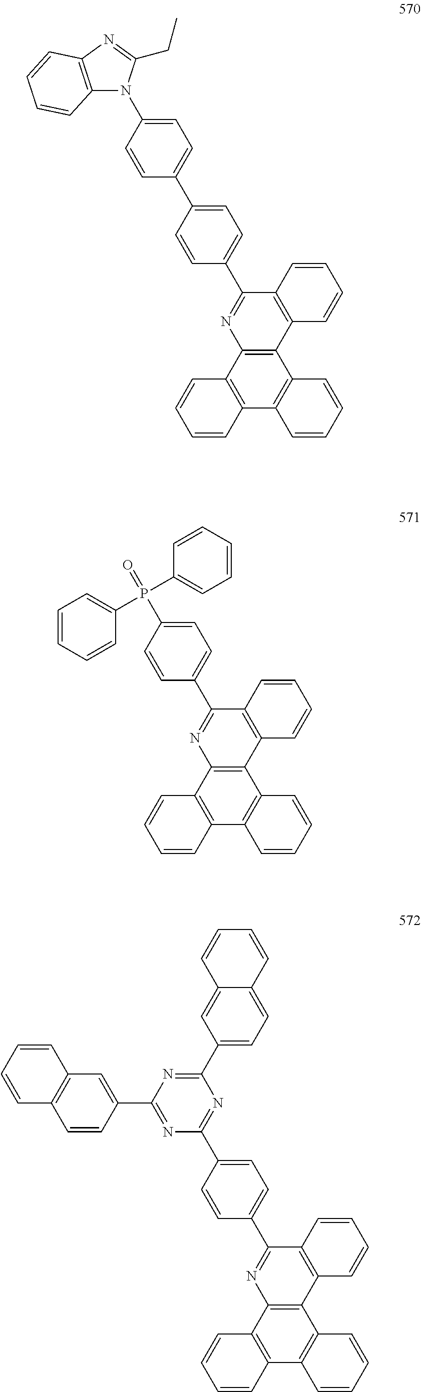 [SMILES: CCc1nc2ccccc2n1-c1ccc(-c2ccc(-c3nc4c5ccccc5c5ccccc5c4c4ccccc34)cc2)cc1 ; O=P(c1ccccc1)(c1ccccc1)c1ccc(-c2nc3c4ccccc4c4ccccc4c3c3ccccc23)cc1 ; c1ccc2cc(-c3nc(-c4ccc(-c5nc6c7ccccc7c7ccccc7c6c6ccccc56)cc4)nc(-c4ccc5ccccc5c4)n3)ccc2c1]

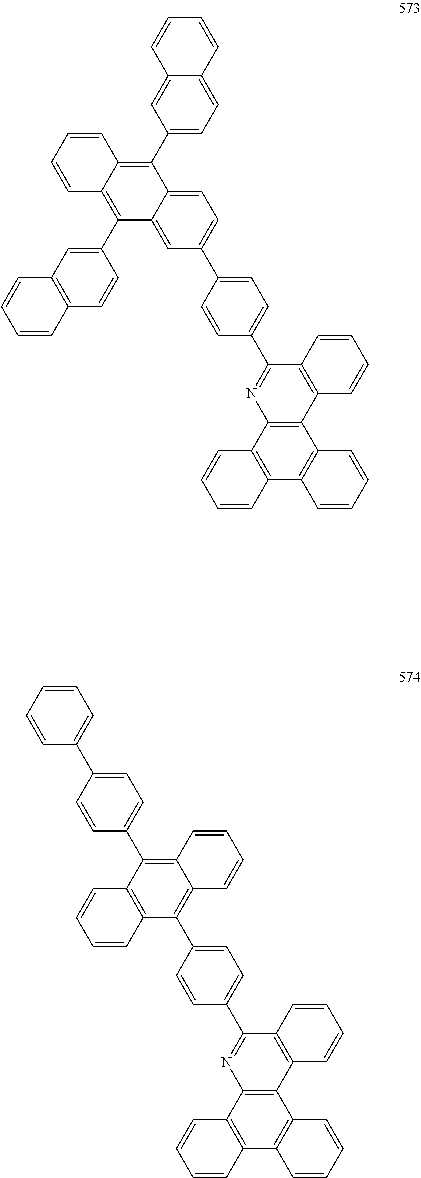 [SMILES: c1ccc2cc(-c3c4ccccc4c(-c4ccc5ccccc5c4)c4cc(-c5ccc(-c6nc7c8ccccc8c8ccccc8c7c7ccccc67)cc5)ccc34)ccc2c1 ; c1ccc(-c2ccc(-c3c4ccccc4c(-c4ccc(-c5nc6c7ccccc7c7ccccc7c6c6ccccc56)cc4)c4ccccc34)cc2)cc1]

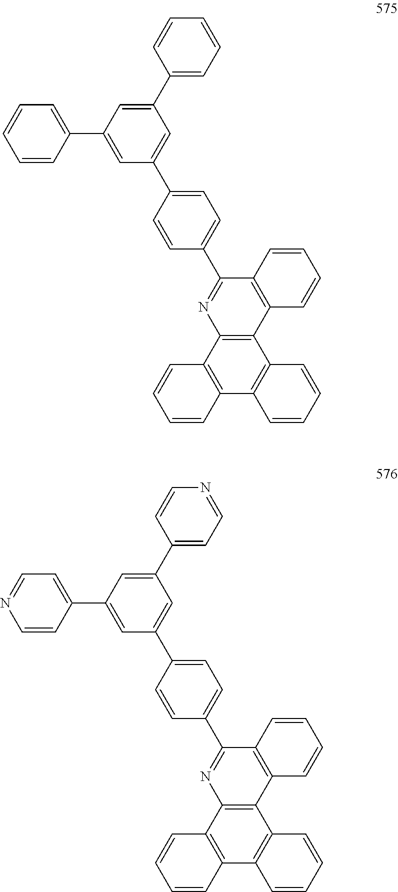 [SMILES: c1ccc(-c2cc(-c3ccccc3)cc(-c3ccc(-c4nc5c6ccccc6c6ccccc6c5c5ccccc45)cc3)c2)cc1 ; c1ccc2c(c1)c(-c1ccc(-c3cc(-c4ccncc4)cc(-c4ccncc4)c3)cc1)nc1c3ccccc3c3ccccc3c21]

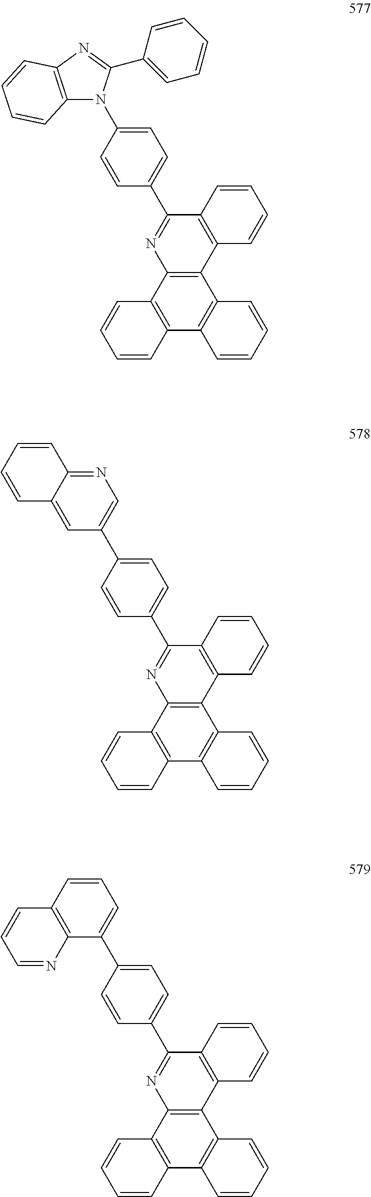 [SMILES: c1ccc(-c2nc3ccccc3n2-c2ccc(-c3nc4c5ccccc5c5ccccc5c4c4ccccc34)cc2)cc1 ; c1ccc2ncc(-c3ccc(-c4nc5c6ccccc6c6ccccc6c5c5ccccc45)cc3)cc2c1 ; c1cnc2c(-c3ccc(-c4nc5c6ccccc6c6ccccc6c5c5ccccc45)cc3)cccc2c1]

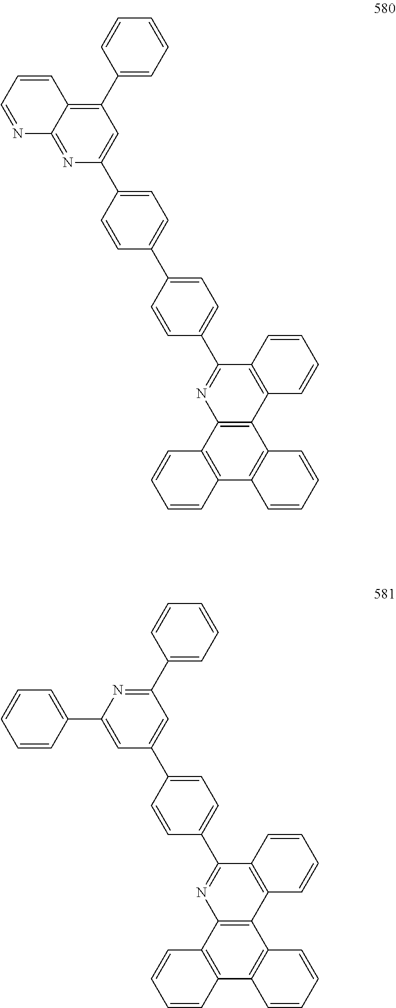 [SMILES: c1ccc(-c2cc(-c3ccc(-c4ccc(-c5nc6c7ccccc7c7ccccc7c6c6ccccc56)cc4)cc3)nc3ncccc23)cc1 ; c1ccc(-c2cc(-c3ccc(-c4nc5c6ccccc6c6ccccc6c5c5ccccc45)cc3)cc(-c3ccccc3)n2)cc1]

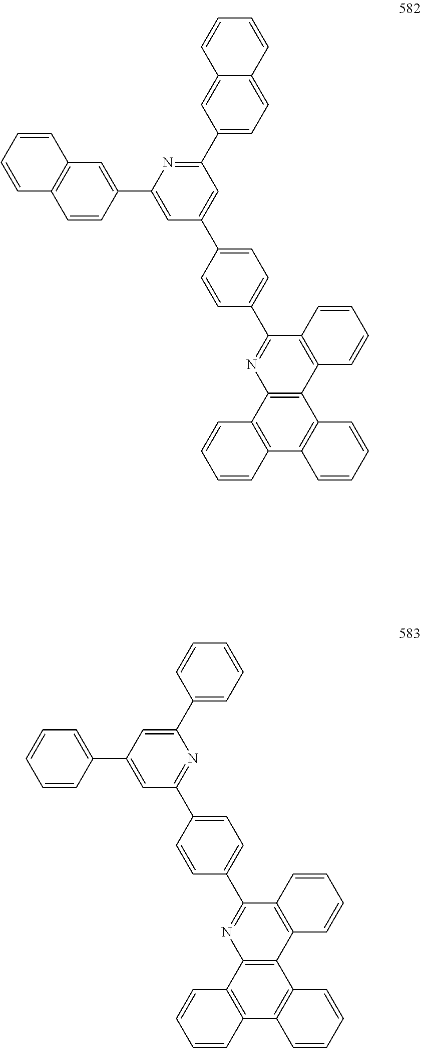 [SMILES: c1ccc2cc(-c3cc(-c4ccc(-c5nc6c7ccccc7c7ccccc7c6c6ccccc56)cc4)cc(-c4ccc5ccccc5c4)n3)ccc2c1 ; c1ccc(-c2cc(-c3ccccc3)nc(-c3ccc(-c4nc5c6ccccc6c6ccccc6c5c5ccccc45)cc3)c2)cc1]

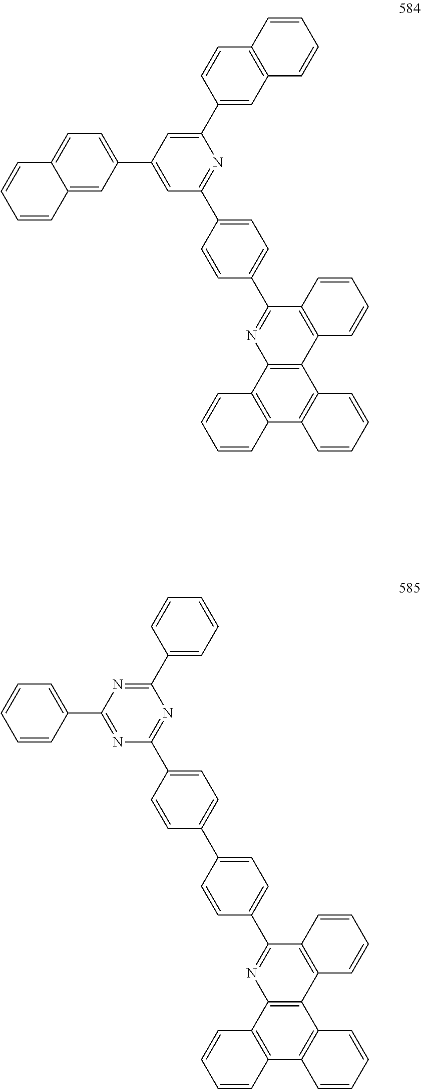 [SMILES: c1ccc2cc(-c3cc(-c4ccc(-c5nc6c7ccccc7c7ccccc7c6c6ccccc56)cc4)nc(-c4ccc5ccccc5c4)c3)ccc2c1 ; c1ccc(-c2nc(-c3ccccc3)nc(-c3ccc(-c4ccc(-c5nc6c7ccccc7c7ccccc7c6c6ccccc56)cc4)cc3)n2)cc1]

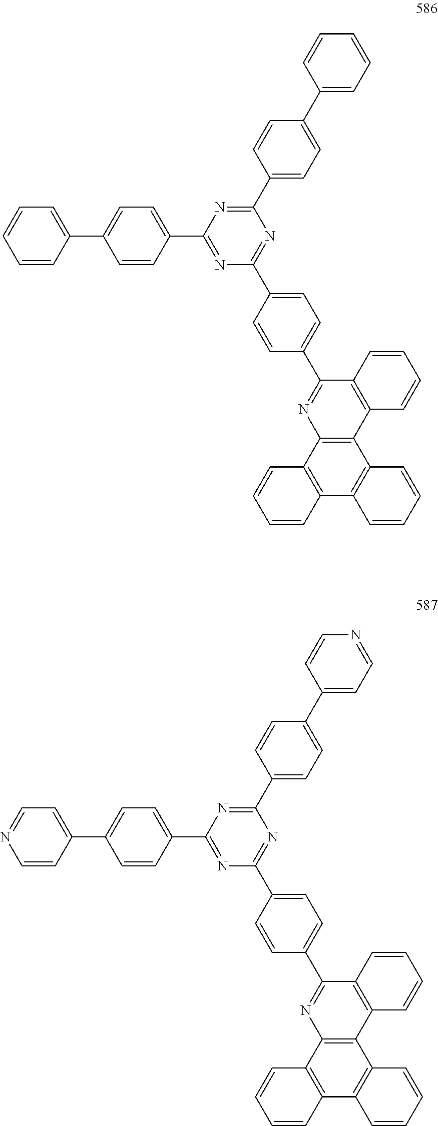 [SMILES: c1ccc(-c2ccc(-c3nc(-c4ccc(-c5ccccc5)cc4)nc(-c4ccc(-c5nc6c7ccccc7c7ccccc7c6c6ccccc56)cc4)n3)cc2)cc1 ; c1ccc2c(c1)c(-c1ccc(-c3nc(-c4ccc(-c5ccncc5)cc4)nc(-c4ccc(-c5ccncc5)cc4)n3)cc1)nc1c3ccccc3c3ccccc3c21]

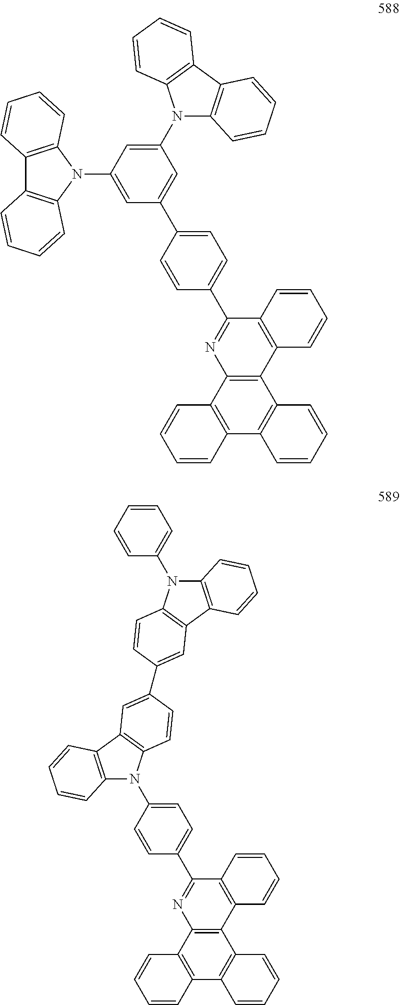 [SMILES: c1ccc2c(c1)c(-c1ccc(-c3cc(-n4c5ccccc5c5ccccc54)cc(-n4c5ccccc5c5ccccc54)c3)cc1)nc1c3ccccc3c3ccccc3c21 ; c1ccc(-n2c3ccccc3c3cc(-c4ccc5c(c4)c4ccccc4n5-c4ccc(-c5nc6c7ccccc7c7ccccc7c6c6ccccc56)cc4)ccc32)cc1]

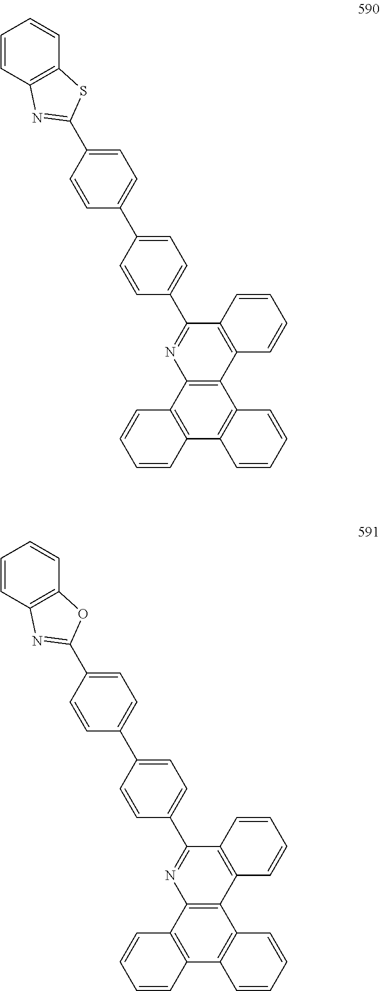 [SMILES: c1ccc2sc(-c3ccc(-c4ccc(-c5nc6c7ccccc7c7ccccc7c6c6ccccc56)cc4)cc3)nc2c1 ; c1ccc2oc(-c3ccc(-c4ccc(-c5nc6c7ccccc7c7ccccc7c6c6ccccc56)cc4)cc3)nc2c1]

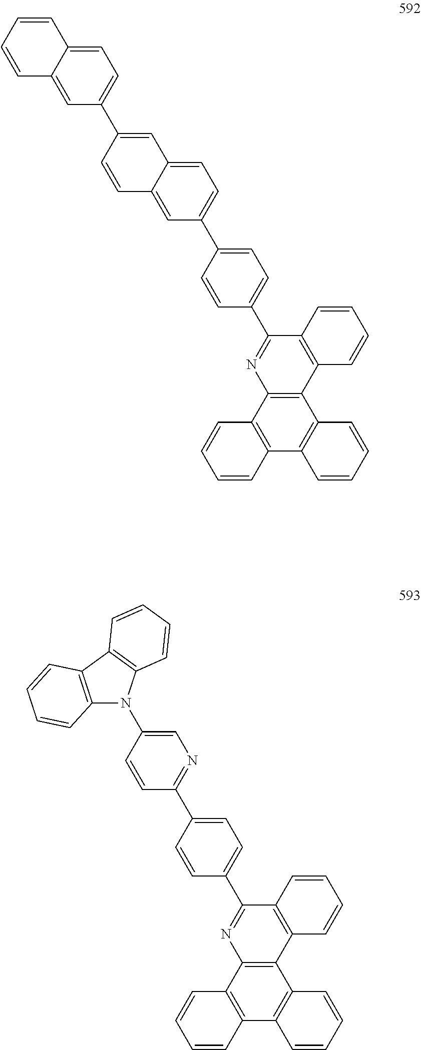 [SMILES: c1ccc2cc(-c3ccc4cc(-c5ccc(-c6nc7c8ccccc8c8ccccc8c7c7ccccc67)cc5)ccc4c3)ccc2c1 ; c1ccc2c(c1)c(-c1ccc(-c3ccc(-n4c5ccccc5c5ccccc54)cn3)cc1)nc1c3ccccc3c3ccccc3c21]

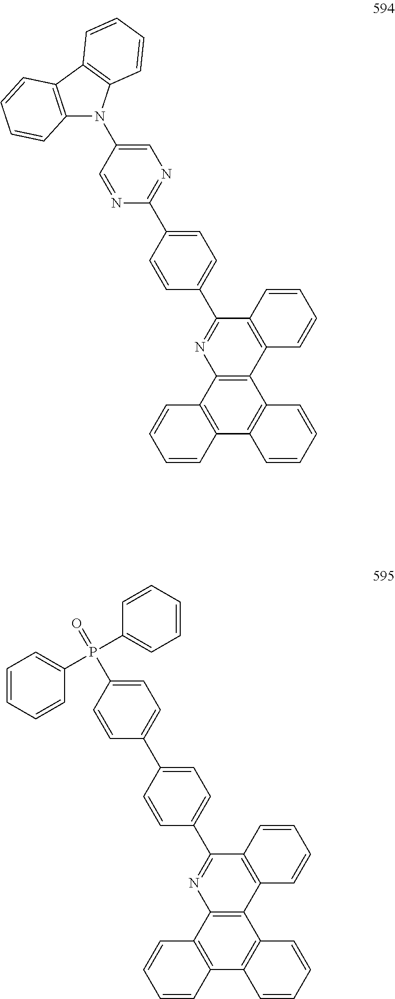 [SMILES: c1ccc2c(c1)c(-c1ccc(-c3ncc(-n4c5ccccc5c5ccccc54)cn3)cc1)nc1c3ccccc3c3ccccc3c21 ; O=P(c1ccccc1)(c1ccccc1)c1ccc(-c2ccc(-c3nc4c5ccccc5c5ccccc5c4c4ccccc34)cc2)cc1]

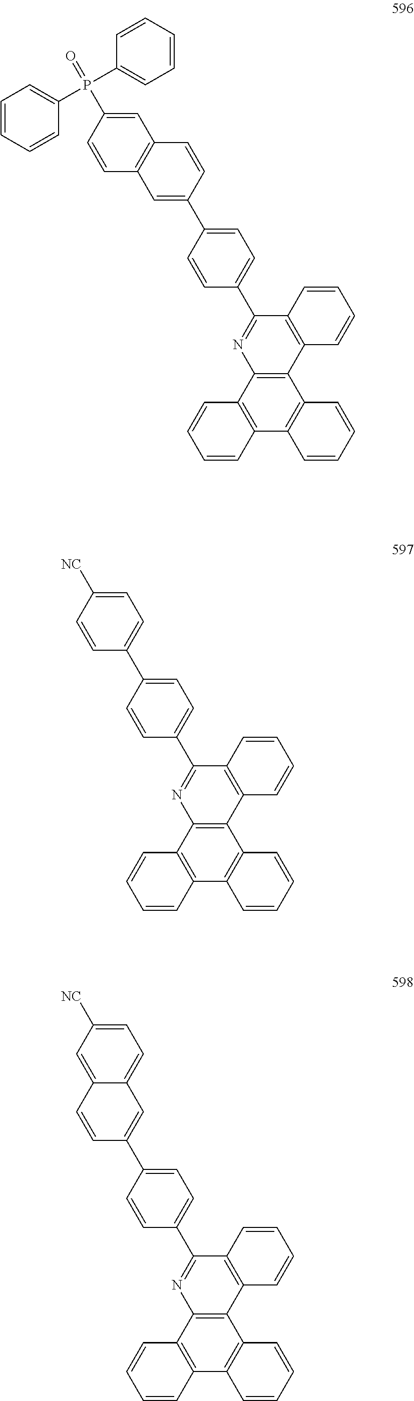 [SMILES: O=P(c1ccccc1)(c1ccccc1)c1ccc2cc(-c3ccc(-c4nc5c6ccccc6c6ccccc6c5c5ccccc45)cc3)ccc2c1 ; N#Cc1ccc(-c2ccc(-c3nc4c5ccccc5c5ccccc5c4c4ccccc34)cc2)cc1 ; N#Cc1ccc2cc(-c3ccc(-c4nc5c6ccccc6c6ccccc6c5c5ccccc45)cc3)ccc2c1]

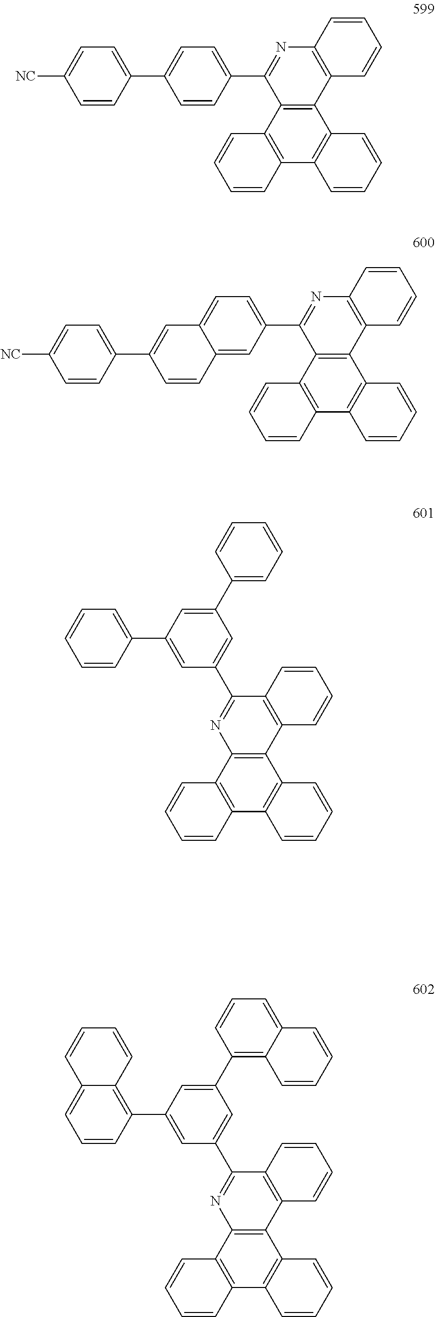 [SMILES: N#Cc1ccc(-c2ccc(-c3nc4ccccc4c4c5ccccc5c5ccccc5c34)cc2)cc1 ; N#Cc1ccc(-c2ccc3cc(-c4nc5ccccc5c5c6ccccc6c6ccccc6c45)ccc3c2)cc1 ; c1ccc(-c2cc(-c3ccccc3)cc(-c3nc4c5ccccc5c5ccccc5c4c4ccccc34)c2)cc1 ; c1ccc2c(-c3cc(-c4cccc5ccccc45)cc(-c4nc5c6ccccc6c6ccccc6c5c5ccccc45)c3)cccc2c1]

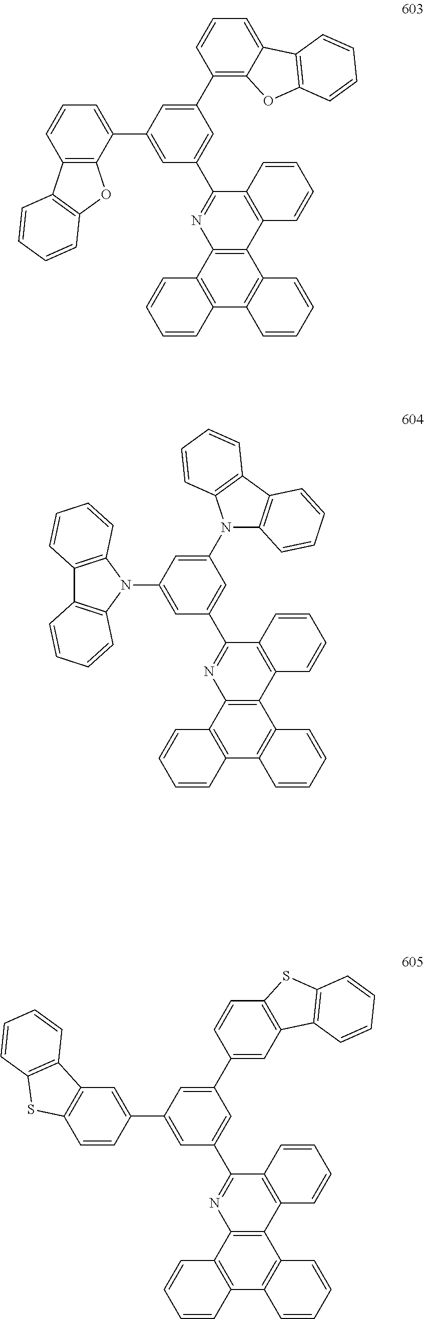 [SMILES: c1ccc2c(c1)oc1c(-c3cc(-c4nc5c6ccccc6c6ccccc6c5c5ccccc45)cc(-c4cccc5c4oc4ccccc45)c3)cccc12 ; c1ccc2c(c1)c(-c1cc(-n3c4ccccc4c4ccccc43)cc(-n3c4ccccc4c4ccccc43)c1)nc1c3ccccc3c3ccccc3c21 ; c1ccc2c(c1)sc1ccc(-c3cc(-c4ccc5sc6ccccc6c5c4)cc(-c4nc5c6ccccc6c6ccccc6c5c5ccccc45)c3)cc12]

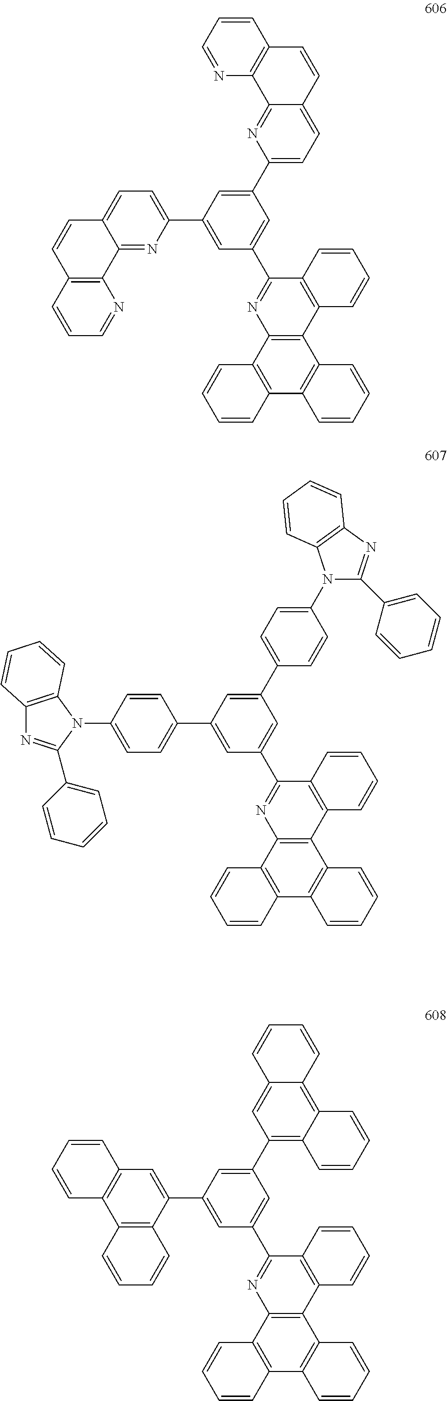 [SMILES: c1cnc2c(c1)ccc1ccc(-c3cc(-c4ccc5ccc6cccnc6c5n4)cc(-c4nc5c6ccccc6c6ccccc6c5c5ccccc45)c3)nc12 ; c1ccc(-c2nc3ccccc3n2-c2ccc(-c3cc(-c4ccc(-n5c(-c6ccccc6)nc6ccccc65)cc4)cc(-c4nc5c6ccccc6c6ccccc6c5c5ccccc45)c3)cc2)cc1 ; c1ccc2c(c1)cc(-c1cc(-c3cc4ccccc4c4ccccc34)cc(-c3nc4c5ccccc5c5ccccc5c4c4ccccc34)c1)c1ccccc12]

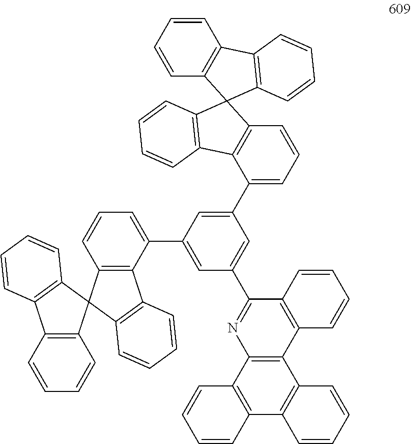 [SMILES: c1ccc2c(c1)-c1ccccc1C21c2ccccc2-c2c(-c3cc(-c4cccc5c4-c4ccccc4C54c5ccccc5-c5ccccc54)cc(-c4nc5c6ccccc6c6ccccc6c5c5ccccc45)c3)cccc21]

16. An organic light emitting device comprising:
an anode;
a cathode; and
one or more organic material layers provided between the anode and the cathode, wherein one or more layers of the organic material layers include the compound of the chemical formula 1 of claim 1.

17. The organic light emitting device of claim 16, wherein an organic material layer including the compound of the chemical formula 1 is one or more layers selected from a hole injection layer, a hole transport layer, a light emitting layer, a hole blocking layer, an electron transport layer, and an electron injection layer.

18. The organic light emitting device of claim 16, wherein an organic material layer including the compound of the chemical formula 1 is an electron transport layer.

19. The organic light emitting device of claim 16, wherein an organic material layer including the compound of the chemical formula 1 is a light emitting layer.

20. The organic light emitting device of claim 16, wherein an organic material layer including the compound of the chemical formula 1 is a hole blocking layer.
